(19) United States
(12) Patent Application Publication
(10) Pub. No.: US 2024/0130227 A1
(43) Pub. Date: Apr. 18, 2024

(54) HETEROCYCLIC COMPOUND, ORGANIC ELECTROLUMINESCENT DEVICE COMPRISING SAME, AND COMPOSITION FOR ORGANIC LAYER

(71) Applicant: LT MATERIALS CO., LTD., Yongin-si, Gyeonggi-do (KR)

(72) Inventors: Dong Jin LEE, Yongin-si (KR); Gi Back LEE, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/268,471

(22) PCT Filed: Nov. 16, 2021
(86) PCT No.: PCT/KR2021/016706

§ 371 Date: Jun. 20, 2023

(30) Foreign Application Priority Data
Dec. 21, 2020 (KR) 10-2020-0179956

(51) Int. Cl. H10K 85/60; C07D 307/91; C07D 333/50; C07D 409/12; C09K 11/06

(52) U.S. Cl. CPC H10K 85/636; C07D 307/91; C07D 333/50; C07D 409/12; C09K 11/06; H10K 85/633; H10K 50/15

(57) ABSTRACT

The present invention provides a heterocyclic compound represented by Formula 1 and an organic light-emitting device comprising the same.

### TECHNICAL FIELD

This application claims the benefit of priority based on Korean Patent Application No. 10-2020-0179956 filed on Dec. 21, 2020, the entire contents of which are incorporated herein as part of the present specification.

The present invention relates to a heterocyclic compound, an organic light-emitting device comprising the same, and a composition for an organic layer of an organic light-emitting device.

### BACKGROUND ART

An organic light-emitting device (organic light-emitting diode; OLED) has recently received a lot of attention due to an increase in demand for flat panel display devices. The organic light-emitting device is a device that converts electrical energy into light, and the performance of the organic light-emitting device is greatly affected by an organic material positioned between electrodes.

The organic light-emitting device has a structure in which an organic thin film is disposed between two electrodes. When a voltage is applied to the organic light-emitting device having such a structure, electrons and holes injected from the two electrodes combine in the organic thin film to form a pair, and then emit light while disappearing. The organic thin film may be composed of a single layer or multiple layers, if necessary.

The material for the organic thin film may have a light-emitting function, if necessary. For example, as a material for the organic thin film, a compound capable of constituting the light-emitting layer by itself may be used, or a compound capable of serving as a host or dopant of the host-dopant-based light-emitting layer may be used. In addition, as a material for the organic thin film, a compound capable of performing the roles of a hole injection layer, a hole transport layer, an electron-blocking layer, a hole-blocking layer, an electron transport layer, an electron injection layer, an electron-generating layer, and the like may be used.

In order to improve the performance, lifetime, or efficiency of the organic light-emitting device, there is a continuous demand for the development of materials for the organic thin film.

### PRIOR ART REFERENCES

### Patent Documents

Korean Patent Application Laying-Open No. 10-2018-0035116

### DISCLOSURE

### Technical Problem

It is an object of the present invention to provide a heterocyclic compound capable of imparting a low driving voltage, excellent luminous efficiency, and excellent lifetime properties to an organic light-emitting device.

It is another object of the present invention to provide an organic light-emitting device comprising the heterocyclic compound.

It is another object of the present invention to provide a composition for an organic layer comprising the heterocyclic compound.

### Technical Solution

The present invention provides a heterocyclic compound represented by following Formula 1:

A heterocyclic compound represented by following Formula 1:

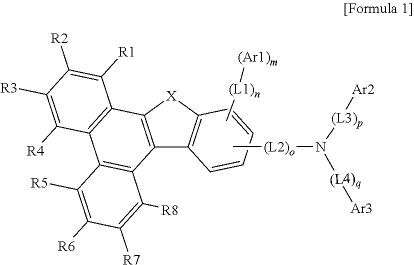

wherein,
X is O or S;
Ar1, Ar2, and Ar3 are the same as or different from each other and are each independently a substituted or unsubstituted C6 to C60 aryl group or a substituted or unsubstituted C2 to C60 heteroaryl group;
R1 to R8 are the same as or different from each other and are each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; or —NR21R22, wherein R21 and R22 are the same as or different from each other and are each independently a substituted or unsubstituted C1 to C10 alkyl group, a substituted or unsubstituted C6 to C60 aryl group, or a substituted or unsubstituted C2 to C60 heteroaryl group; and R21 and R22 above may be combined with each other to form a substituted or unsubstituted C6 to C60 aromatic hydrocarbon ring or a substituted or unsubstituted C2 to C60 heterocycle;
L1 to L4 are the same as or different from each other and are each independently a direct bond, a substituted or unsubstituted C6 to C60 arylene group, or a substituted or unsubstituted C2 to C60 heteroarylene group,
m is an integer from 1 to 3, with the proviso that when m is 2 or more, each Ar1 is the same as or different from each other;
n, o, p, and q are the same as or different from each other and are each independently an integer from 0 to 3, with the proviso that when each of n, o, p and q is 2 or more, each of L1, L2, L3 and L4 is the same as or different from each other.

In addition, the present invention provides an organic light-emitting device comprising:
a first electrode;
a second electrode provided to face the first electrode; and
one or more organic layers provided between the first electrode and the second electrode,
wherein the organic layers comprise the heterocyclic compound represented by Formula 1.

In addition, the present invention provides a composition for an organic layer of an organic light-emitting device, comprising the heterocyclic compound represented by Formula 1.

### Advantageous Effects

The heterocyclic compound of the present invention and the composition for an organic layer comprising the same may be usefully used as a material for an organic layer of an organic light-emitting device. In particular, these are used as a material for a hole transport layer and/or a material for an electron-blocking layer, thereby providing remarkable effects of lowering the driving voltage, improving the luminous efficiency, and improving the lifetime properties, of the organic light-emitting device. In addition, the heterocyclic compound of the present invention provides excellent thermal stability.

The organic light-emitting device of the present invention comprises the heterocyclic compound, thereby providing excellent driving voltage, luminous efficiency, and lifestime properties.

### DESCRIPTION OF DRAWINGS

FIGS. 1 to 3 are drawings schematically showing a stacked structure of an organic light-emitting device according to one embodiment of the present invention, respectively.

### BEST MODE

Hereinafter, the present invention will be described in detail.

In the present invention, the term “substituted” means that a hydrogen atom bonded to a carbon atom of a compound is replaced with another substituent, and the position to be substituted is not limited as long as it is the position at which a hydrogen atom is substituted, that is, the position at which it may be substituted with a substituent. When substituted with two or more substituents, the two or more substituents may be the same as or different from each other.

In the present invention, the term “substituted or unsubstituted” means that it is unsubstituted or substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″, —P(═O)RR′; C1 to C20 alkylamine; C6 to C60 monocyclic or polycyclic arylamine; and C2 to C60 monocyclic or polycyclic heteroarylamine, or that it is unsubstituted or substituted with a substituent in which two or more substituents selected from the above-exemplified substituents are connected to each other.

In the present invention, the alkyl group includes a linear or branched chain having 1 to 60 carbon atoms, and may be further substituted with another substituent. The number of carbon atoms in the alkyl group may be 1 to 60, specifically 1 to 40, more specifically 1 to 20. Specific examples include, but are not limited to, a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like.

In the present invention, the alkenyl group includes a linear or branched chain having 2 to 60 carbon atoms, and may be further substituted with another substituent. The number of carbon atoms in the alkenyl group may be 2 to 60, specifically 2 to 40, more specifically 2 to 20. Specific examples include, but are not limited to, a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like.

In the present invention, the alkynyl group includes a linear or branched chain having 2 to 60 carbon atoms, and may be further substituted with another substituent. The number of carbon atoms in the alkynyl group may be 2 to 60, specifically 2 to 40, more specifically 2 to 20.

In the present invention, the cycloalkyl group includes a monocyclic or polycyclic ring having 3 to 60 carbon atoms, and may be further substituted with another substituent. In this case, the polycyclic ring refers to a group in which a cycloalkyl group is directly connected or condensed with another cyclic group. In this case, another cyclic group may be a cycloalkyl group, but may be a different type of cyclic group, for example, a heterocycloalkyl group, an aryl group, a heteroaryl group, or the like. The number of carbon atoms in the cycloalkyl group may be 3 to 60, specifically 3 to 40, more specifically 5 to 20. Specifically, it includes, but is not limited to, a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, 4-tert-butylcyclohexyl group, a cycloheptyl group, a cyclooctyl group, and the like.

In the present invention, the heterocycloalkyl group includes O, S, Se, N, or, Si as a heteroatom, includes a monocyclic or polycyclic ring having 2 to 60 carbon atoms, and may be further substituted with another substituent. In this case, the polycyclic group refers to a group in which a heterocycloalkyl group is directly connected or condensed with another cyclic group. In this case, another cyclic group may be a heterocycloalkyl group, but may be a different type of cyclic group, for example, a cycloalkyl group, an aryl group, a heteroaryl group, or the like. The number of carbon atoms in the heterocycloalkyl group may be 2 to 60, specifically 2 to 40, more specifically 3 to 20.

In the present invention, the aryl group includes a monocyclic or polycyclic ring having 6 to 60 carbon atoms, and may be further substituted with other substituents. In this case, the polycyclic ring refers to a group in which an aryl group is directly connected or condensed with another cyclic group. In this case, another cyclic group may be an aryl group, but may be a different type of cyclic group, for example, a cycloalkyl group, a heterocycloalkyl group, a heteroaryl group, or the like. The aryl group includes a spiro group. The number of carbon atoms in the aryl group may be 6 to 60, specifically 6 to 40, more specifically 6 to 25. Specific examples of the aryl group may include, but are not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, tetracenyl group, a pentacenyl group, a fluorenyl group, an indenyl group, an acenaphthylenyl group, a benzofluorenyl group, a spirobifluorenyl group, a 2,3-dihydro-1H-indenyl group, condensed cyclic groups thereof, and the like.

In the present invention, the fluorenyl group may be substituted, and adjacent substituents may be bonded to each other to form a ring.

When the fluorenyl group is substituted, it may be, but is not limited to,

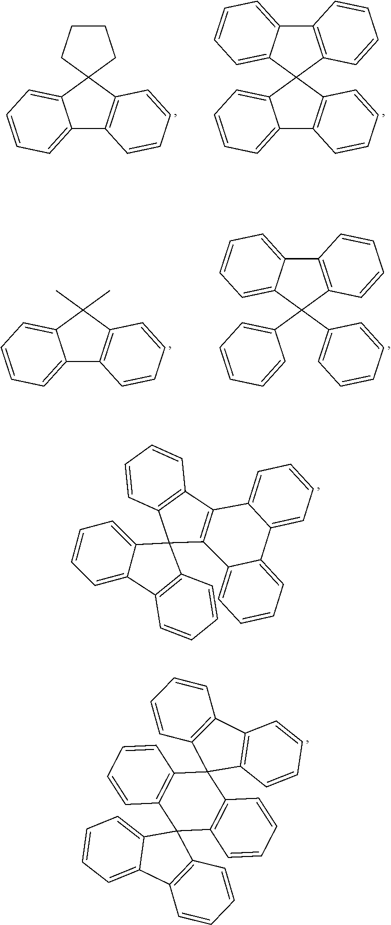

or the like.

In the present invention, the heteroaryl group includes S, O, Se, N, or Si as a heteroatom, includes a monocyclic or polycyclic ring having 2 to 60 carbon atoms, and may be further substituted with other substituents. In this case, the polycyclic group refers to a group in which a heteroaryl group is directly connected or condensed with another cyclic group. In this case, another cyclic group may be a heteroaryl group, but may be a different type of cyclic group, for example, a cycloalkyl group, a heterocycloalkyl group, an aryl group, or the like. The number of carbon atoms in the heteroaryl group may be 2 to 60, specifically 2 to 40, more specifically 3 to 25. Specific examples of the heteroaryl group may include, but are not limited to, a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolylyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilol group, a spirobi(dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c] [1,2,5]thiadiazolyl group, a 5,10-dihydrodibenzo[b,e] [1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like.

In the present invention, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but is preferably 1 to 30. Specific examples of the amine group include, but are not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like.

In the present invention, the arylene group refers to a group having two bonding positions on the aryl group, that is, a divalent group. The above description of the aryl group may be applied, except that each of them is a divalent group. In addition, the heteroarylene group refers to a group having two bonding positions on the heteroaryl group, that is, a divalent group. The above description of the heteroaryl group may be applied, except that each of them is a divalent group.

In the present invention, an “adjacent” group may refer to a substituent substituted on an atom directly connected to the atom on which that related substituent is substituted, a substituent which is sterically closest to that substituent, or another substituent substituted on the atom on which related substituent is substituted. For example, two substituents substituted at an ortho position on a benzene ring and two substituents substituted at the same carbon on an aliphatic ring may be interpreted as “adjacent” groups to each other.

In the present invention, “when a substituent is not indicated in the chemical formula or compound structure” means that a hydrogen atom is bonded to a carbon atom. However, since deuterium (²H) is an isotope of hydrogen, some hydrogen atoms may be deuterium.

In one embodiment of the present invention, “when a substituent is not indicated in the chemical formula or compound structure” may mean that hydrogen or deuterium is present at all positions that may be substituted with a substituent. That is, deuterium is an isotope of hydrogen, and thus, some hydrogen atoms may be deuterium that is an isotope, and in this case, the content of deuterium may be 0% to 100%.

In one embodiment of the present invention, in the case of “when a substituent is not indicated in the chemical formula or compound structure,” hydrogen and deuterium may be used interchangeably in compounds unless deuterium is explicitly excluded, such as “the content of deuterium is 0%,” “the content of hydrogen is 100%,” and “all substituents are hydrogen”.

In one embodiment of the present invention, deuterium is one of the isotopes of hydrogen and is an element having a deuteron consisting of one proton and one neutron as a nucleus, and may be expressed as hydrogen-2, and its element symbol may also be written as D or 2H.

In one embodiment of the present invention, an isotope refers to an atom having the same atomic number (Z) but a different mass number (A), and may also be interpreted as an element having the same number of protons but a different number of neutrons.

In one embodiment of the present invention, the meaning of the T % content of a specific substituent may be defined as an equation: T2/T1×100=T %, wherein T1 is defined as the total number of substituents that the basic compound can have and T2 is defined as the number of specific substituents substituted among them.

That is, in one example, the 20% content of deuterium in the phenyl group represented by

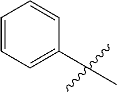 [SMILES: *c1ccccc1]

may mean that the total number of substituents that the phenyl group can have is 5 (T1 in the equation) and the number of deuterium among them is 1 (T2 in the equation). That is, the 20% content of deuterium in the phenyl group may be represented by the following structural formulas:

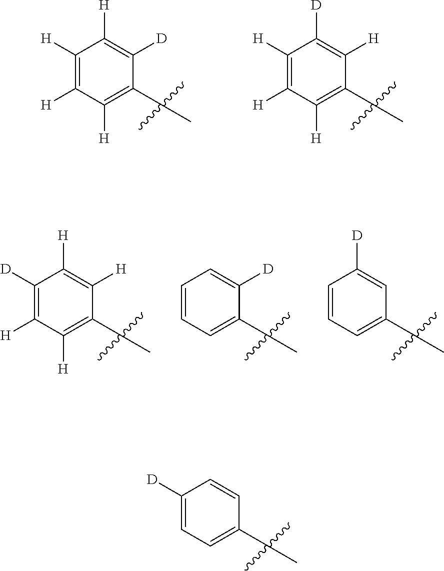 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present invention, the case of “a phenyl group having a deuterium content of 0%” may mean a phenyl group that does not contain deuterium atoms, that is, a phenyl group having 5 hydrogen atoms.

In the present invention, the content of deuterium in the heterocyclic compound represented by Formula 1 may be 0 to 100%.

The present invention provides a heterocyclic compound represented by following Formula 1:

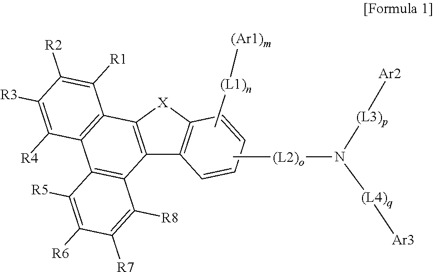

wherein,
X is O or S;
Ar1, Ar2, and Ar3 are the same as or different from each other and are each independently a substituted or unsubstituted C6 to C60 aryl group or a substituted or unsubstituted C2 to C60 heteroaryl group,
R1 to R8 are the same as or different from each other and are each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; or —NR21R22, wherein R21 and R22 are the same as or different from each other and are each independently a substituted or unsubstituted C1 to C10 alkyl group, a substituted or unsubstituted C6 to C60 aryl group, or a substituted or unsubstituted C2 to C60 heteroaryl group; and R21 and R22 above may be combined with each other to form a substituted or unsubstituted C6 to C60 aromatic hydrocarbon ring or a substituted or unsubstituted C2 to C60 heterocycle,
L1 to L4 are the same as or different from each other and are each independently a direct bond, a substituted or unsubstituted C6 to C60 arylene group, or a substituted or unsubstituted C2 to C60 heteroarylene group,
m is an integer from 1 to 3, with the proviso that when m is 2 or more, each Ar1 is the same as or different from each other,
n, o, p, and q are the same as or different from each other and are each independently an integer from 0 to 3, with the proviso that when each of n, o, p and q is 2 or more, each of L1, L2, L3 and L4 is the same as or different from each other.

In one embodiment of the present invention, the heteroatom in the heteroatom-containing substituent may be one or more selected from O, S, Se, N, and Si.

In another embodiment of the present invention, the heteroatom in the heteroatom-containing substituent may be one or more selected from O, S, and N.

In one embodiment of the present invention, X may be 0, and in another embodiment may be S.

In one embodiment of the present invention, Ar1, Ar2, and R3 above may be the same as or different from each other and may be each independently a substituted or unsubstituted C6 to C30 aryl group or a substituted or unsubstituted C2 to C30 heteroaryl group.

In another embodiment of the present invention, Ar1, Ar2, and R3 may be the same as or different from each other and may be each independently a substituted or unsubstituted C6 to C20 aryl group or a substituted or unsubstituted C2 to C20 heteroaryl group.

In another embodiment of the present invention, Ar1, Ar2, and Ar3 may be the same as or different from each other, and may be each independently a substituted or unsubstituted phenyl, naphthalenyl, biphenyl, terphenyl, anthracenyl, phenanthrenyl, pyrenyl, triphenylenyl, carbazolyl, dibenzofuranyl, dibenzothiophenyl, 9,9′-dimethylfluorenyl, 9,9′-dibenzofluorenyl, or 9,9′-spirobifluorene group.

In one embodiment of the present invention, R1 to R8 above may be the same as or different from each other and may be each independently hydrogen, deuterium, halogen, a cyano group, a substituted or unsubstituted C1 to C30 alkyl group, a substituted or unsubstituted C1 to C30 alkoxy group, a substituted or unsubstituted C6 to C60 aryl group, a substituted or unsubstituted C2 to C60 heteroaryl group, or —NR21R22, wherein R21 and R22 may be the same as or different from each other and may be each independently a substituted or unsubstituted C1 to C10 alkyl group, a substituted or unsubstituted C6 to C60 aryl group, or a substituted or unsubstituted C2 to C60 heteroaryl group; and R21 and R22 above may be combined with each other to form a substituted or unsubstituted C6 to C30 aromatic hydrocarbon ring or a substituted or unsubstituted C2 to C30 heterocycle, and
in another embodiment of the present invention, R1 to R8 above may be the same as or different from each other and may be each independently hydrogen, deuterium, halogen, a cyano group, a substituted or unsubstituted C1 to C20 alkyl group, a substituted or unsubstituted C6 to C30 aryl group, a substituted or unsubstituted C2 to C30 heteroaryl group, or —NR21R22, wherein R21 and R22 may be the same as or different from each other and may be each independently a substituted or unsubstituted C1 to C10 alkyl group, a substituted or unsubstituted C6 to C30 aryl group, or a substituted or unsubstituted C2 to C30 heteroaryl group; and R21 and R22 above may be combined with each other to form a substituted or unsubstituted C6 to C30 aromatic hydrocarbon ring or a substituted or unsubstituted C2 to C30 heterocycle.

In another embodiment of the present invention, R1 to R8 above may be the same as or different from each other and may be each independently hydrogen, deuterium, a substituted or unsubstituted C6 to C20 aryl group, a substituted or unsubstituted C2 to C20 heteroaryl group, or —NR21R22, wherein R21 and R22 may be the same as or different from each other and may be each independently a substituted or unsubstituted phenyl, naphthalenyl, biphenyl, terphenyl, anthracenyl, phenanthrenyl, pyrenyl, triphenylenyl, carbazolyl, dibenzofuranyl, dibenzothiophenyl, 9,9′-dimethylfluorenyl, 9,9′-dibenzofluorenyl, or 9,9′-spirobifluorene group.

In another embodiment of the present invention, R1 to R8 above may be the same as or different from each other and may be each independently hydrogen, deuterium, a substituted or unsubstituted phenyl, naphthalenyl, biphenyl, terphenyl, anthracenyl, phenanthrenyl, pyrenyl, triphenylenyl, carbazolyl, dibenzofuranyl, dibenzothiophenyl, 9,9′-dimethylfluorenyl, 9,9′-dibenzofluorenyl, or 9,9′-spirobifluorene group.

In another embodiment of the present invention, R1 to R8 above may be the same as or different from each other and may be hydrogen or deuterium.

In one embodiment of the present invention, L1 to L4 above may be the same as or different from each other and may be each independently a substituted or unsubstituted C6 to C30 aryl group or a substituted or unsubstituted C2 to C30 heteroaryl group.

In another embodiment of the present invention, L1 to L4 above may be the same as or different from each other and may be each independently a substituted or unsubstituted C6 to C20 aryl group or a substituted or unsubstituted C2 to C20 heteroaryl group.

In another embodiment of the present invention, L above may be a substituted or unsubstituted phenylene, naphthalene, anthracenylene, phenanthrene, pyridine, or pyrimidine group.

In one embodiment of the present invention, the ‘substitution’ in the definitions of Ar1, Ar2, and Ar3; R1 to R8; and L1 to L4 may be each independently made with one or more substituents selected from the group consisting of C1 to C10 linear or branched alkyl; C2 to C10 linear or branched alkenyl; C2 to C10 linear or branched alkynyl; C3 to C15 cycloalkyl; C2 to C20 heterocycloalkyl; C6 to C30 aryl; C2 to C30 heteroaryl; C1 to C10 alkylamine; C6 to C30 arylamine; and C2 to C30 heteroarylamine.

In another embodiment of the present invention, the ‘substitution’ in the definitions of Ar1, Ar2, and Ar3; R1 to R8; and L1 to L4 may be each independently made with one or more substituents selected from the group consisting of C1 to C10 linear or branched alkyl; C2 to C10 linear or branched alkenyl; C2 to C10 linear or branched alkynyl; C6 to C30 aryl; C2 to C30 heteroaryl; C6 to C30 arylamine; and C2 to C30 heteroarylamine.

In another embodiment of the present invention, the ‘substitution’ in the definitions of Ar1, Ar2, and Ar3; R1 to R8; and L1 to L4 may be each independently made with one or more substituents selected from the group consisting of C6 to C30 aryl; C2 to C30 heteroaryl; C6 to C30 arylamine; and C2 to C30 heteroarylamine.

In another embodiment of the present invention, the ‘substitution’ in the definitions of Ar1, Ar2, and Ar3; R1 to R8; and L1 to L4 may be each independently made with one or more substituents selected from the group consisting of phenyl, naphthalenyl, pyridinyl, anthracenyl, carbazole, biphenyl, dibenzothiophene, dibenzofuran, and phenanthrenyl.

In one embodiment of the present invention, m may be an integer from 1 to 2, with the proviso that when m is 2, each Ar1 may be each independently selected.

In another embodiment of the present invention, m may be 1.

In one embodiment of the present invention, n, o, p, and q may be the same as or different from each other and may be each independently an integer from 0 to 2, with the proviso that when each of n, o, p and q is 2, each of L1, L2, L3, and L4 is the same as or different from each other.

In another embodiment of the present invention, n, o, p, and q may be the same as or different from each other and may be each independently 0 or 1.

In one embodiment of the present invention, the heterocyclic compound represented by Formula 1 may be a compound represented by any one of following Formulas 2 to 5:

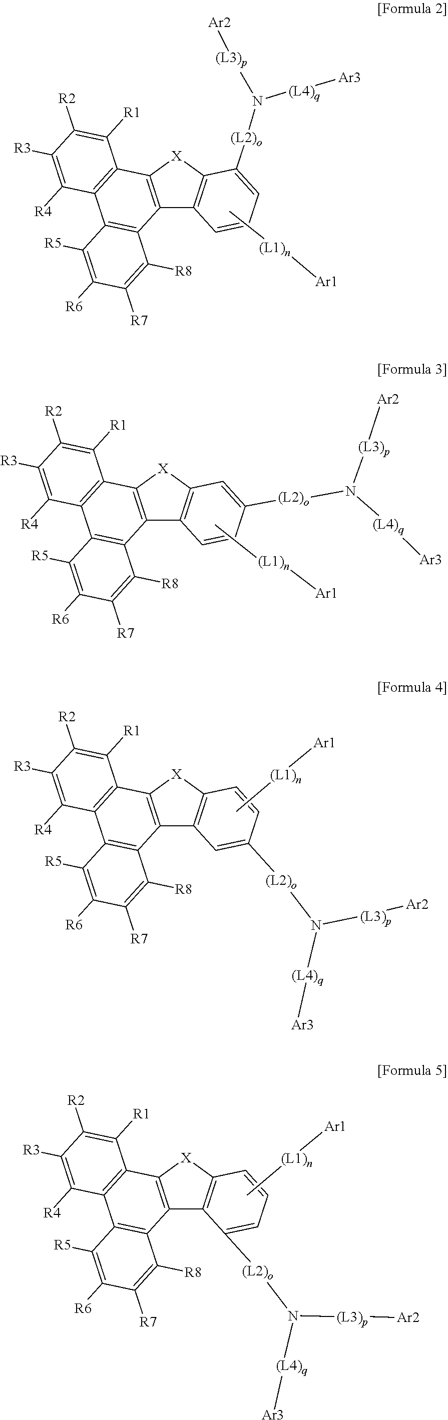

wherein,
X, Ar1, Ar2, Ar3, R1 to R8, L1 to L4, n, o, p, and q are as defined in Formulas 1.

In one embodiment of the present invention, the heterocyclic compound represented by Formula 1 may be a compound represented by any one of the following compounds:

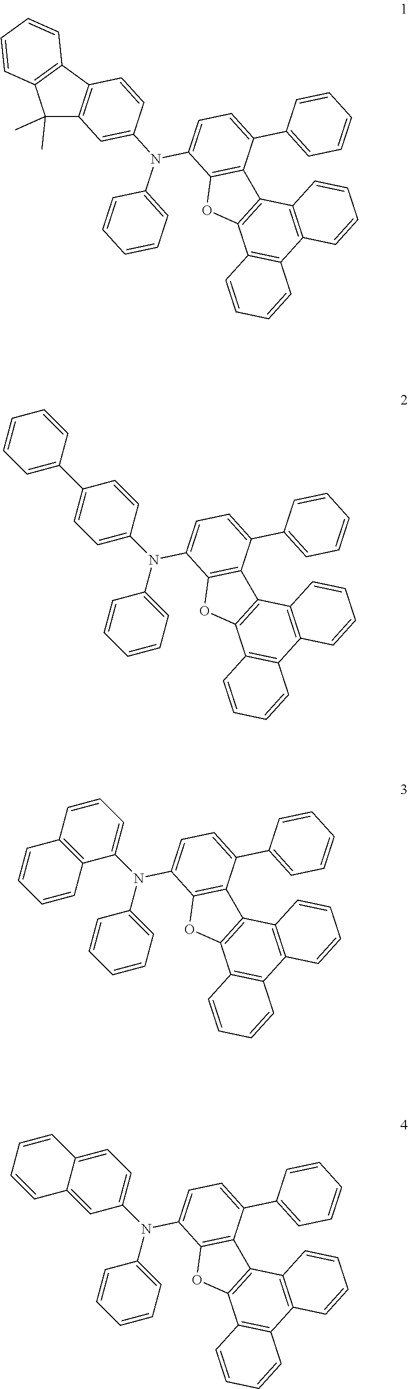 [SMILES: CC1(C)c2ccccc2-c2ccc(N(c3ccccc3)c3ccc(-c4ccccc4)c4c3oc3c5ccccc5c5ccccc5c34)cc21 ; c1ccc(-c2ccc(N(c3ccccc3)c3ccc(-c4ccccc4)c4c3oc3c5ccccc5c5ccccc5c34)cc2)cc1 ; c1ccc(-c2ccc(N(c3ccccc3)c3cccc4ccccc34)c3oc4c5ccccc5c5ccccc5c4c23)cc1 ; c1ccc(-c2ccc(N(c3ccccc3)c3ccc4ccccc4c3)c3oc4c5ccccc5c5ccccc5c4c23)cc1]

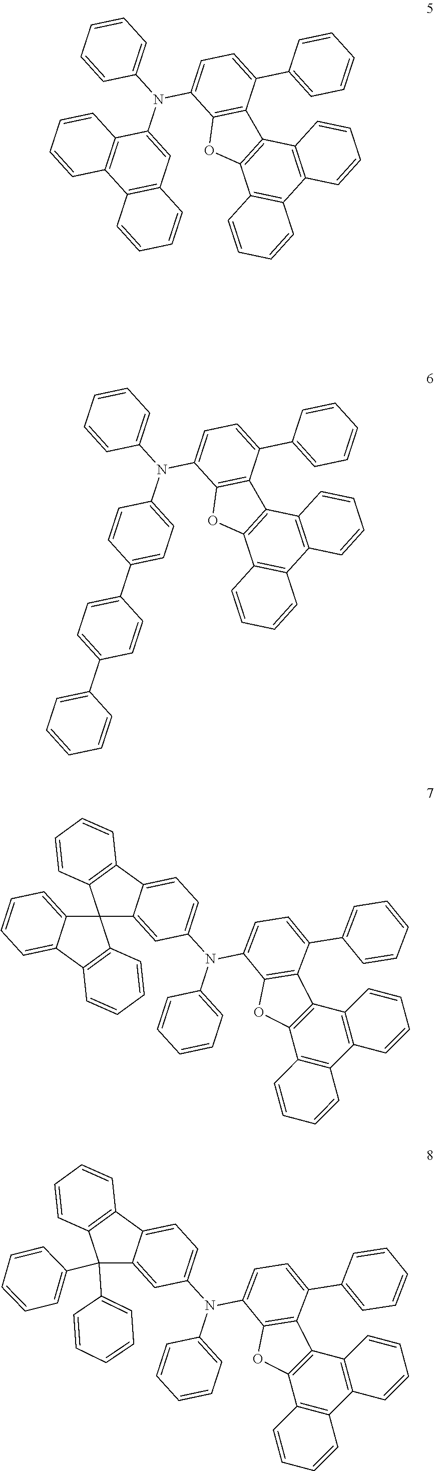 [SMILES: c1ccc(-c2ccc(N(c3ccccc3)c3cc4ccccc4c4ccccc34)c3oc4c5ccccc5c5ccccc5c4c23)cc1 ; c1ccc(-c2ccc(-c3ccc(N(c4ccccc4)c4ccc(-c5ccccc5)c5c4oc4c6ccccc6c6ccccc6c45)cc3)cc2)cc1 ; c1ccc(-c2ccc(N(c3ccccc3)c3ccc4c(c3)C3(c5ccccc5-c5ccccc53)c3ccccc3-4)c3oc4c5ccccc5c5ccccc5c4c23)cc1 ; c1ccc(-c2ccc(N(c3ccccc3)c3ccc4c(c3)C(c3ccccc3)(c3ccccc3)c3ccccc3-4)c3oc4c5ccccc5c5ccccc5c4c23)cc1]

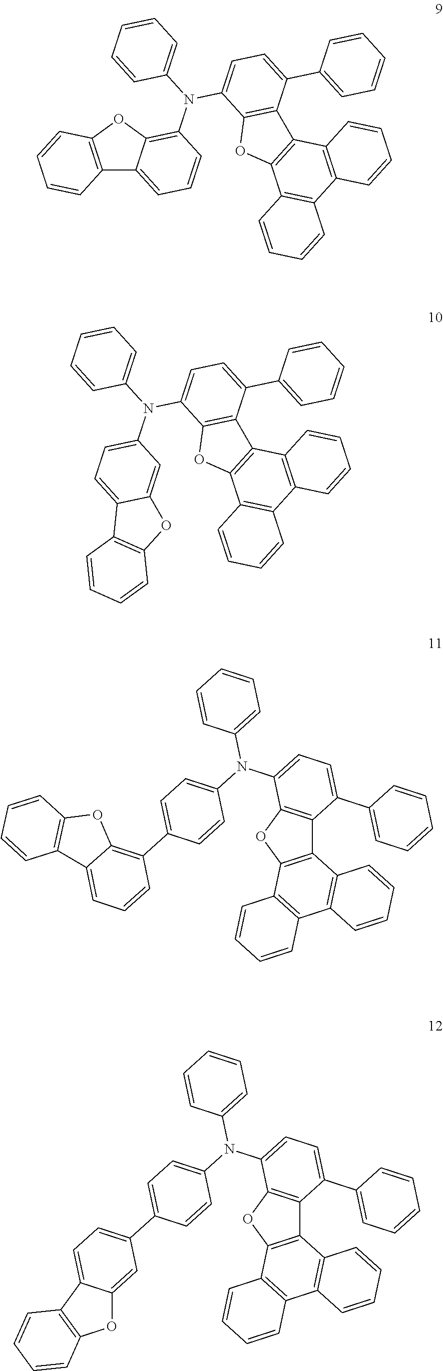 [SMILES: c1ccc(-c2ccc(N(c3ccccc3)c3cccc4c3oc3ccccc34)c3oc4c5ccccc5c5ccccc5c4c23)cc1 ; c1ccc(-c2ccc(N(c3ccccc3)c3ccc4c(c3)oc3ccccc34)c3oc4c5ccccc5c5ccccc5c4c23)cc1 ; c1ccc(-c2ccc(N(c3ccccc3)c3ccc(-c4cccc5c4oc4ccccc45)cc3)c3oc4c5ccccc5c5ccccc5c4c23)cc1 ; c1ccc(-c2ccc(N(c3ccccc3)c3ccc(-c4ccc5c(c4)oc4ccccc45)cc3)c3oc4c5ccccc5c5ccccc5c4c23)cc1]

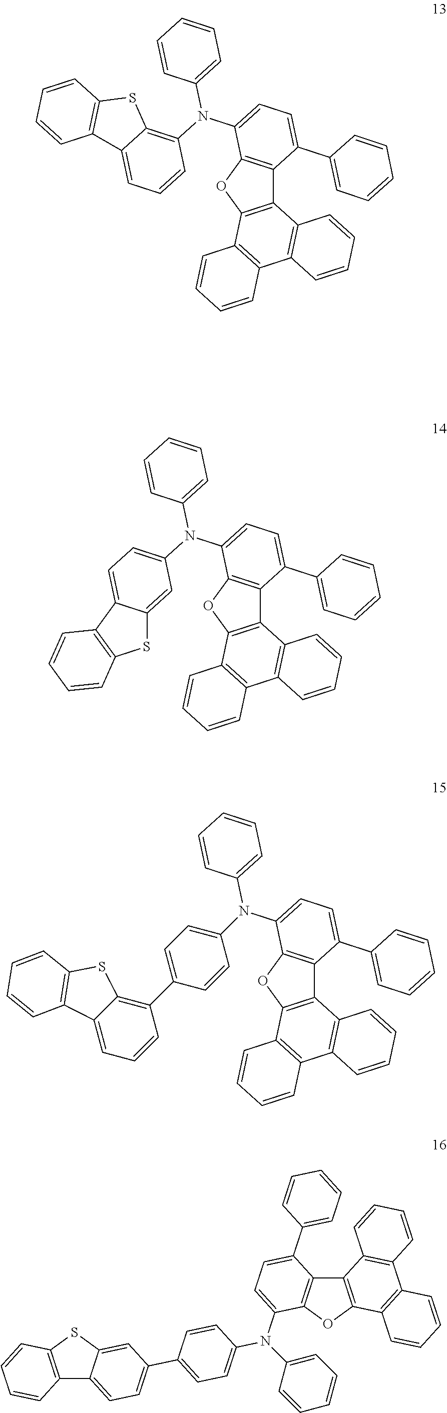 [SMILES: c1ccc(-c2ccc(N(c3ccccc3)c3cccc4c3sc3ccccc34)c3oc4c5ccccc5c5ccccc5c4c23)cc1 ; c1ccc(-c2ccc(N(c3ccccc3)c3ccc4c(c3)sc3ccccc34)c3oc4c5ccccc5c5ccccc5c4c23)cc1 ; c1ccc(-c2ccc(N(c3ccccc3)c3ccc(-c4cccc5c4sc4ccccc45)cc3)c3oc4c5ccccc5c5ccccc5c4c23)cc1 ; c1ccc(-c2ccc(N(c3ccccc3)c3ccc(-c4ccc5c(c4)sc4ccccc45)cc3)c3oc4c5ccccc5c5ccccc5c4c23)cc1]

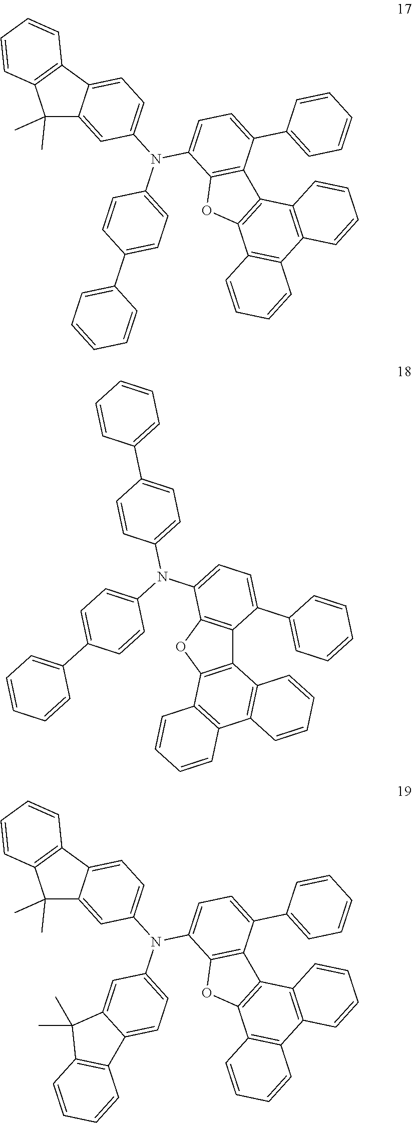

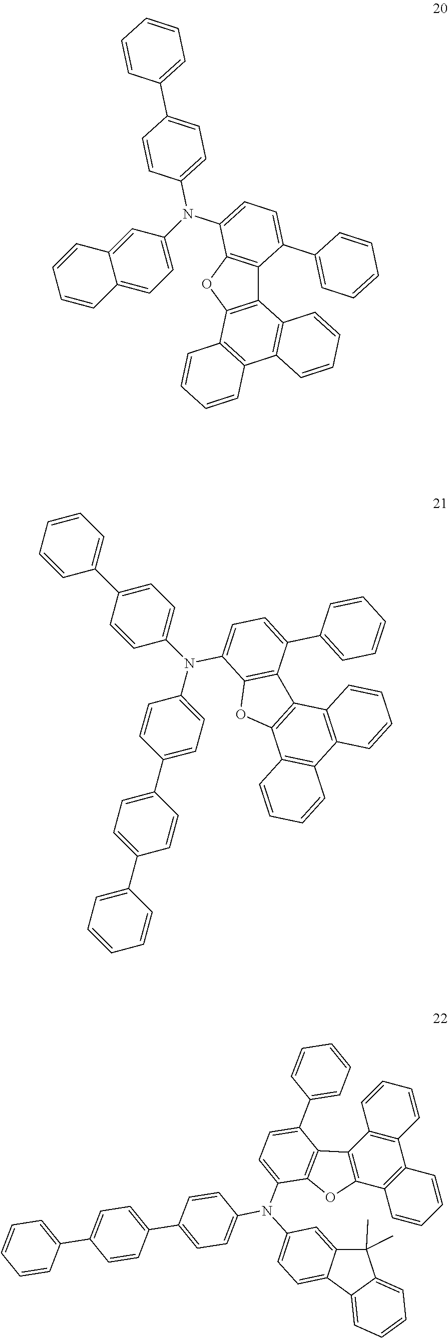 [SMILES: c1ccc(-c2ccc(N(c3ccc4ccccc4c3)c3ccc(-c4ccccc4)c4c3oc3c5ccccc5c5ccccc5c34)cc2)cc1 ; c1ccc(-c2ccc(-c3ccc(N(c4ccc(-c5ccccc5)cc4)c4ccc(-c5ccccc5)c5c4oc4c6ccccc6c6ccccc6c45)cc3)cc2)cc1 ; CC1(C)c2ccccc2-c2ccc(N(c3ccc(-c4ccc(-c5ccccc5)cc4)cc3)c3ccc(-c4ccccc4)c4c3oc3c5ccccc5c5ccccc5c34)cc21]

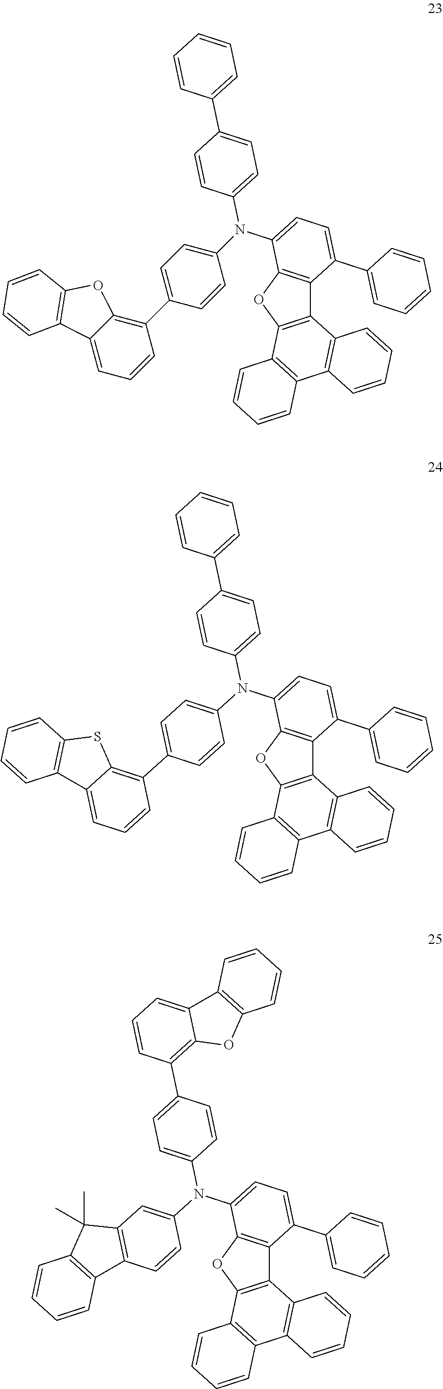 [SMILES: c1ccc(-c2ccc(N(c3ccc(-c4cccc5c4oc4ccccc45)cc3)c3ccc(-c4ccccc4)c4c3oc3c5ccccc5c5ccccc5c34)cc2)cc1 ; c1ccc(-c2ccc(N(c3ccc(-c4cccc5c4sc4ccccc45)cc3)c3ccc(-c4ccccc4)c4c3oc3c5ccccc5c5ccccc5c34)cc2)cc1 ; CC1(C)c2ccccc2-c2ccc(N(c3ccc(-c4cccc5c4oc4ccccc45)cc3)c3ccc(-c4ccccc4)c4c3oc3c5ccccc5c5ccccc5c34)cc21]

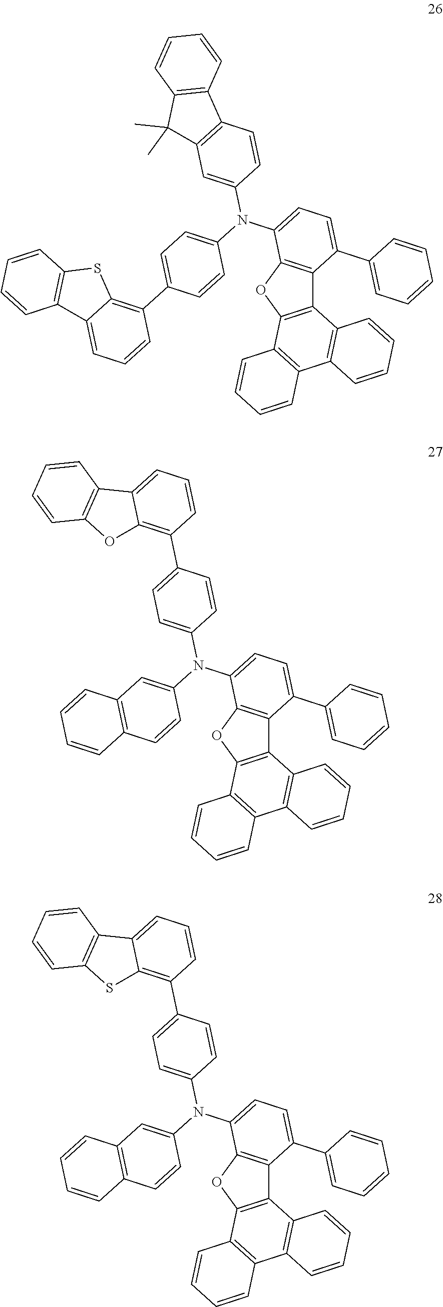 [SMILES: CC1(C)c2ccccc2-c2ccc(N(c3ccc(-c4cccc5c4sc4ccccc45)cc3)c3ccc(-c4ccccc4)c4c3oc3c5ccccc5c5ccccc5c34)cc21 ; c1ccc(-c2ccc(N(c3ccc(-c4cccc5c4oc4ccccc45)cc3)c3ccc4ccccc4c3)c3oc4c5ccccc5c5ccccc5c4c23)cc1 ; c1ccc(-c2ccc(N(c3ccc(-c4cccc5c4sc4ccccc45)cc3)c3ccc4ccccc4c3)c3oc4c5ccccc5c5ccccc5c4c23)cc1]

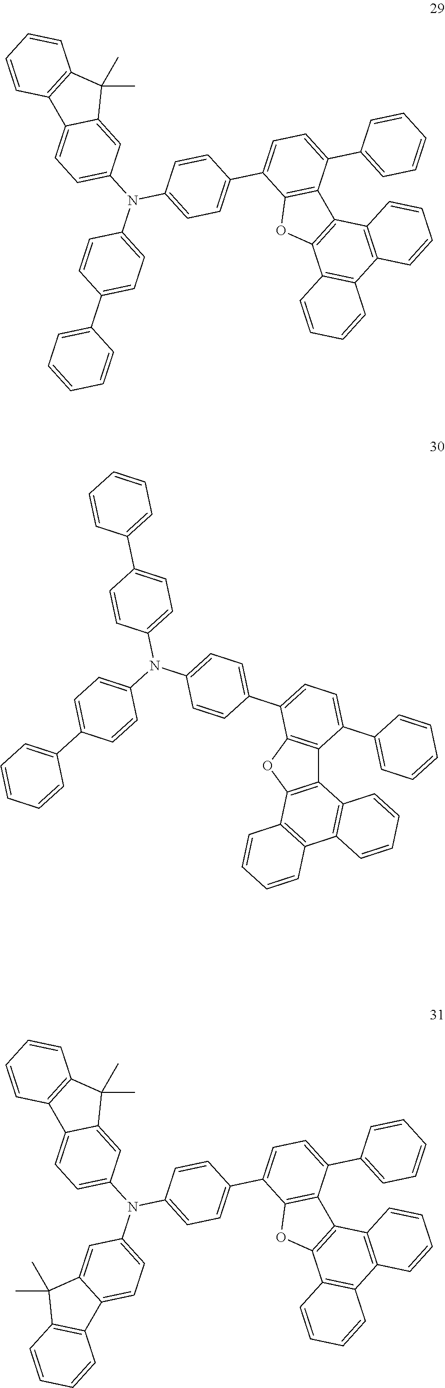 [SMILES: CC1(C)c2ccccc2-c2ccc(N(c3ccc(-c4ccccc4)cc3)c3ccc(-c4ccc(-c5ccccc5)c5c4oc4c6ccccc6c6ccccc6c45)cc3)cc21 ; c1ccc(-c2ccc(N(c3ccc(-c4ccccc4)cc3)c3ccc(-c4ccc(-c5ccccc5)c5c4oc4c6ccccc6c6ccccc6c45)cc3)cc2)cc1 ; CC1(C)c2ccccc2-c2ccc(N(c3ccc(-c4ccc(-c5ccccc5)c5c4oc4c6ccccc6c6ccccc6c45)cc3)c3ccc4c(c3)C(C)(C)c3ccccc3-4)cc21]

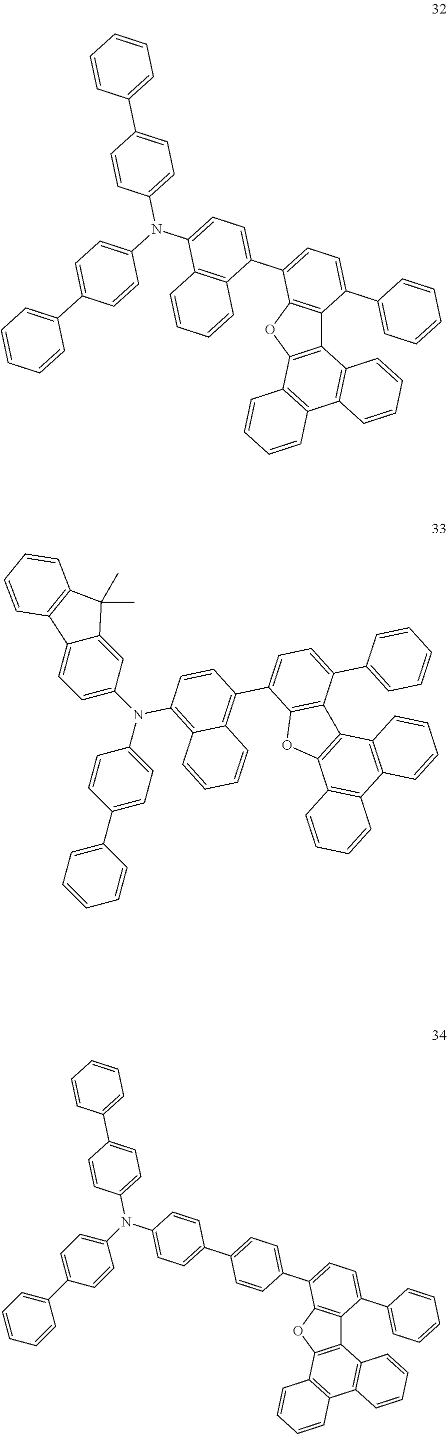 [SMILES: c1ccc(-c2ccc(N(c3ccc(-c4ccccc4)cc3)c3ccc(-c4ccc(-c5ccccc5)c5c4oc4c6ccccc6c6ccccc6c45)c4ccccc34)cc2)cc1 ; CC1(C)c2ccccc2-c2ccc(N(c3ccc(-c4ccccc4)cc3)c3ccc(-c4ccc(-c5ccccc5)c5c4oc4c6ccccc6c6ccccc6c45)c4ccccc34)cc21 ; c1ccc(-c2ccc(N(c3ccc(-c4ccccc4)cc3)c3ccc(-c4ccc(-c5ccc(-c6ccccc6)c6c5oc5c7ccccc7c7ccccc7c56)cc4)cc3)cc2)cc1]

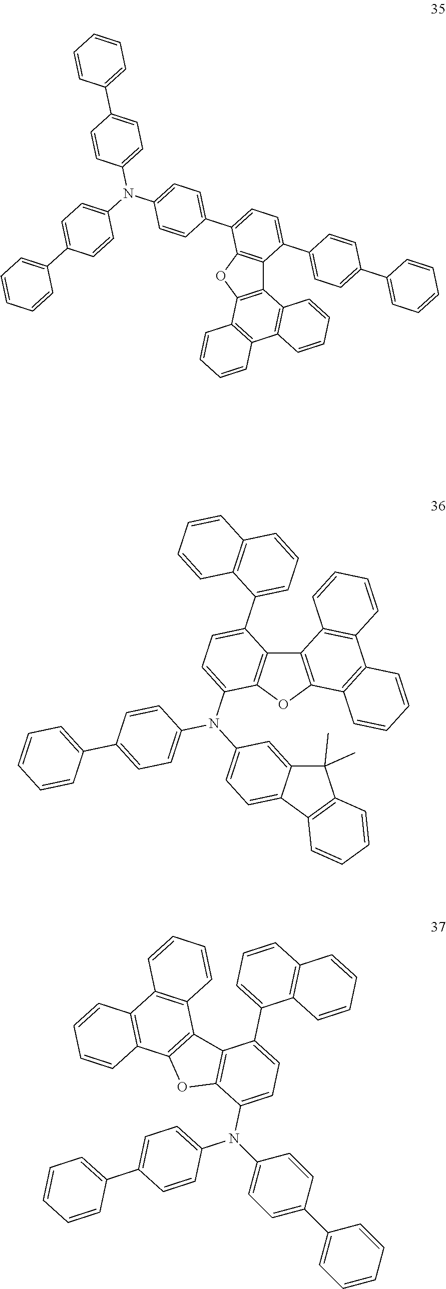 [SMILES: c1ccc(-c2ccc(-c3ccc(-c4ccc(N(c5ccc(-c6ccccc6)cc5)c5ccc(-c6ccccc6)cc5)cc4)c4oc5c6ccccc6c6ccccc6c5c34)cc2)cc1 ; CC1(C)c2ccccc2-c2ccc(N(c3ccc(-c4ccccc4)cc3)c3ccc(-c4cccc5ccccc45)c4c3oc3c5ccccc5c5ccccc5c34)cc21 ; c1ccc(-c2ccc(N(c3ccc(-c4ccccc4)cc3)c3ccc(-c4cccc5ccccc45)c4c3oc3c5ccccc5c5ccccc5c34)cc2)cc1]

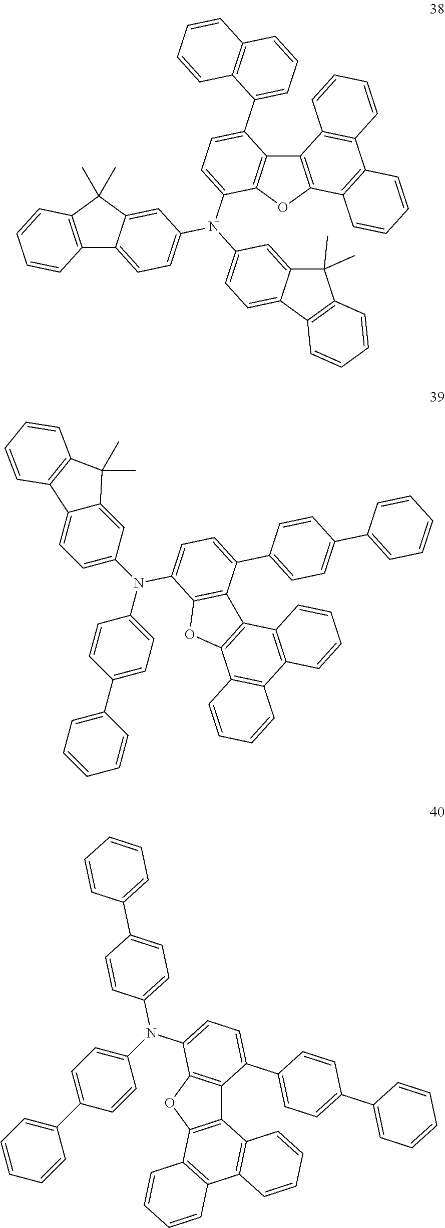 [SMILES: CC1(C)c2ccccc2-c2ccc(N(c3ccc4c(c3)C(C)(C)c3ccccc3-4)c3ccc(-c4cccc5ccccc45)c4c3oc3c5ccccc5c5ccccc5c34)cc21 ; CC1(C)c2ccccc2-c2ccc(N(c3ccc(-c4ccccc4)cc3)c3ccc(-c4ccc(-c5ccccc5)cc4)c4c3oc3c5ccccc5c5ccccc5c34)cc21 ; c1ccc(-c2ccc(-c3ccc(N(c4ccc(-c5ccccc5)cc4)c4ccc(-c5ccccc5)cc4)c4oc5c6ccccc6c6ccccc6c5c34)cc2)cc1]

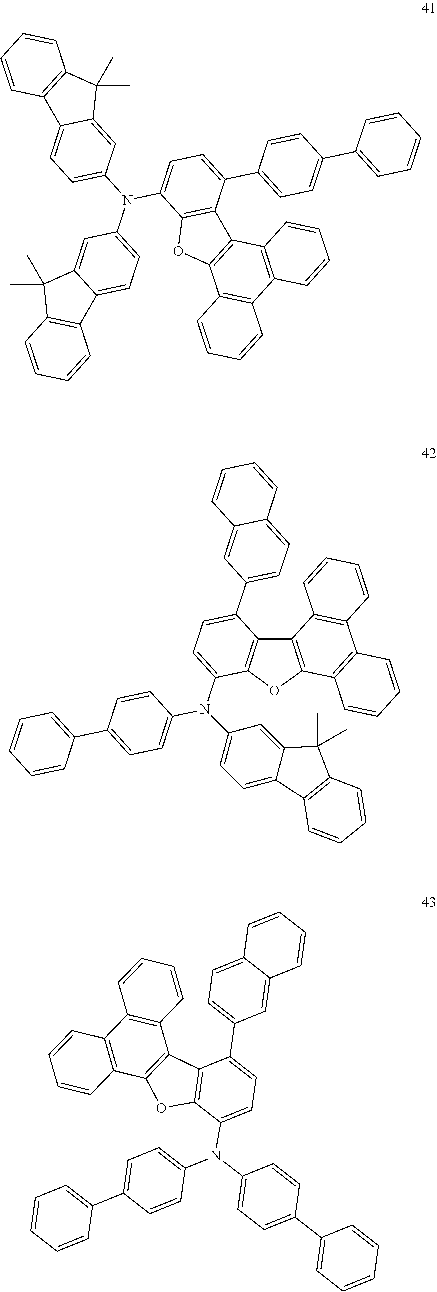 [SMILES: CC1(C)c2ccccc2-c2ccc(N(c3ccc4c(c3)C(C)(C)c3ccccc3-4)c3ccc(-c4ccc(-c5ccccc5)cc4)c4c3oc3c5ccccc5c5ccccc5c34)cc21 ; CC1(C)c2ccccc2-c2ccc(N(c3ccc(-c4ccccc4)cc3)c3ccc(-c4ccc5ccccc5c4)c4c3oc3c5ccccc5c5ccccc5c34)cc21 ; c1ccc(-c2ccc(N(c3ccc(-c4ccccc4)cc3)c3ccc(-c4ccc5ccccc5c4)c4c3oc3c5ccccc5c5ccccc5c34)cc2)cc1]

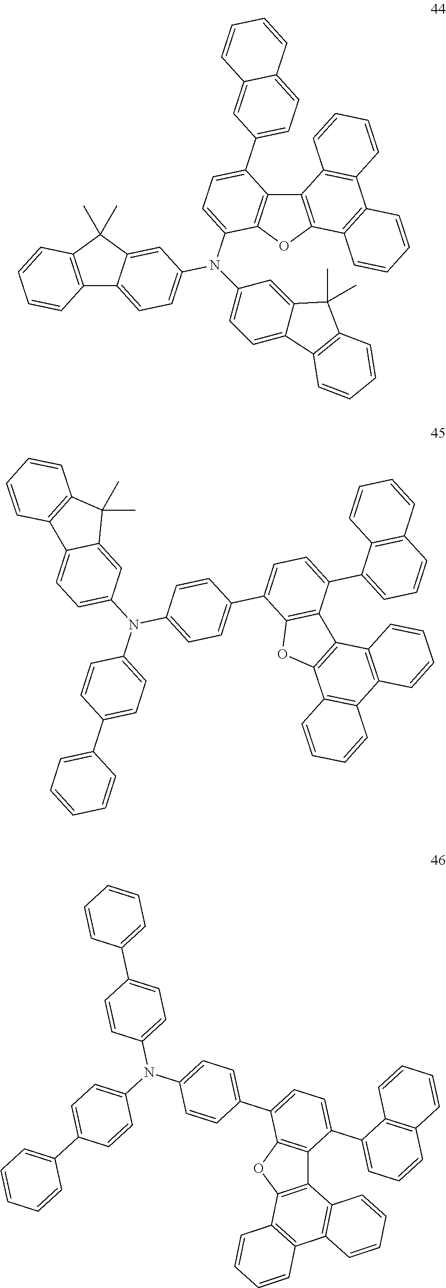 [SMILES: CC1(C)c2ccccc2-c2ccc(N(c3ccc4c(c3)C(C)(C)c3ccccc3-4)c3ccc(-c4ccc5ccccc5c4)c4c3oc3c5ccccc5c5ccccc5c34)cc21 ; CC1(C)c2ccccc2-c2ccc(N(c3ccc(-c4ccccc4)cc3)c3ccc(-c4ccc(-c5cccc6ccccc56)c5c4oc4c6ccccc6c6ccccc6c45)cc3)cc21 ; c1ccc(-c2ccc(N(c3ccc(-c4ccccc4)cc3)c3ccc(-c4ccc(-c5cccc6ccccc56)c5c4oc4c6ccccc6c6ccccc6c45)cc3)cc2)cc1]

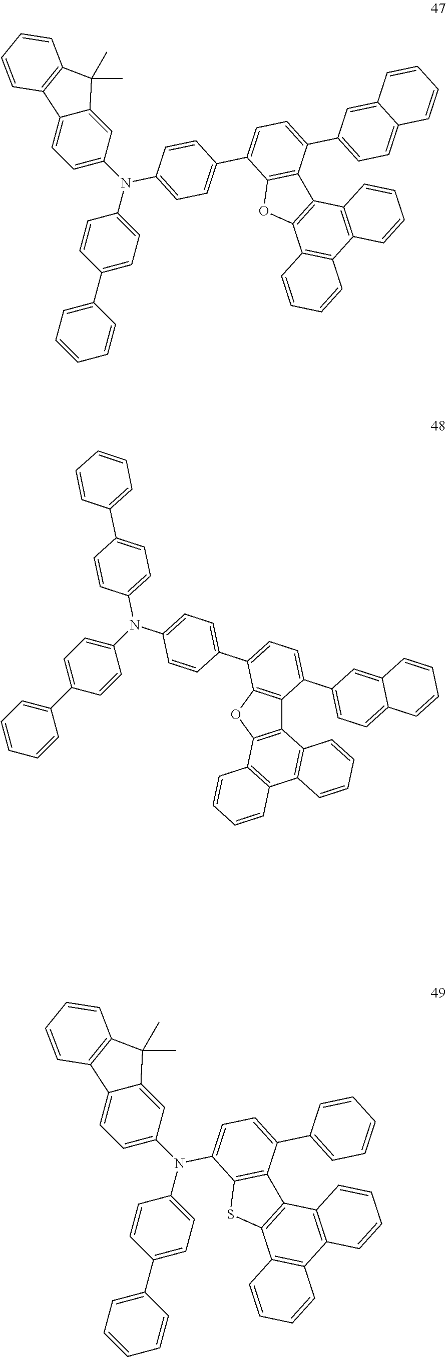 [SMILES: CC1(C)c2ccccc2-c2ccc(N(c3ccc(-c4ccccc4)cc3)c3ccc(-c4ccc(-c5ccc6ccccc6c5)c5c4oc4c6ccccc6c6ccccc6c45)cc3)cc21 ; c1ccc(-c2ccc(N(c3ccc(-c4ccccc4)cc3)c3ccc(-c4ccc(-c5ccc6ccccc6c5)c5c4oc4c6ccccc6c6ccccc6c45)cc3)cc2)cc1 ; CC1(C)c2ccccc2-c2ccc(N(c3ccc(-c4ccccc4)cc3)c3ccc(-c4ccccc4)c4c3sc3c5ccccc5c5ccccc5c34)cc21]

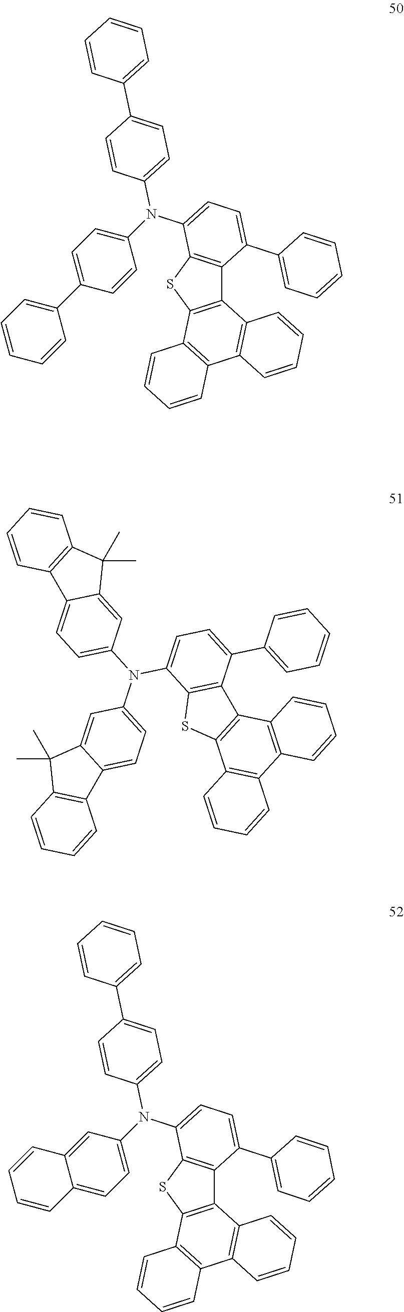 [SMILES: c1ccc(-c2ccc(N(c3ccc(-c4ccccc4)cc3)c3ccc(-c4ccccc4)c4c3sc3c5ccccc5c5ccccc5c34)cc2)cc1 ; CC1(C)c2ccccc2-c2ccc(N(c3ccc4c(c3)C(C)(C)c3ccccc3-4)c3ccc(-c4ccccc4)c4c3sc3c5ccccc5c5ccccc5c34)cc21 ; c1ccc(-c2ccc(N(c3ccc4ccccc4c3)c3ccc(-c4ccccc4)c4c3sc3c5ccccc5c5ccccc5c34)cc2)cc1]

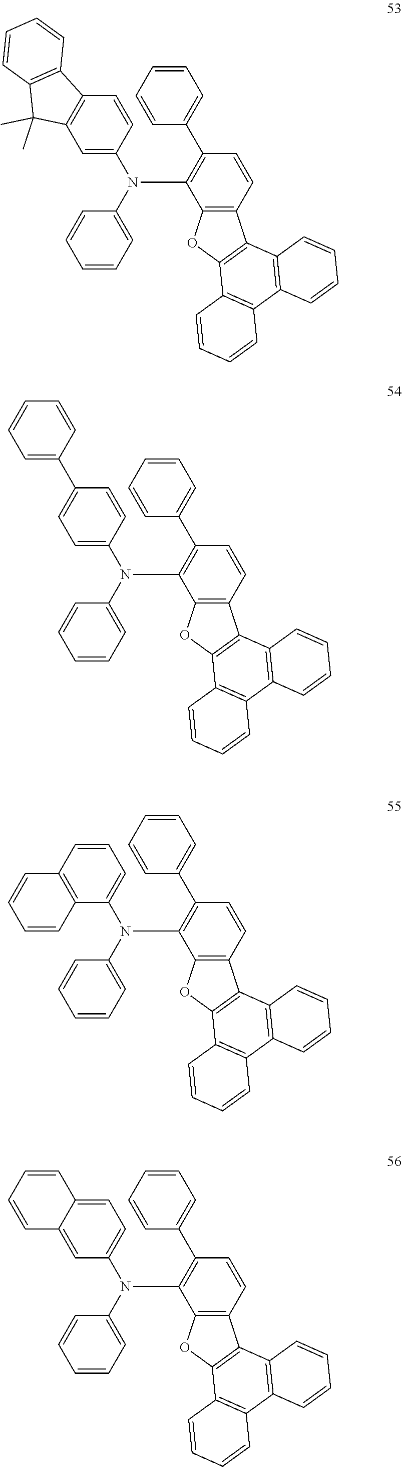 [SMILES: CC1(C)c2ccccc2-c2ccc(N(c3ccccc3)c3c(-c4ccccc4)ccc4c3oc3c5ccccc5c5ccccc5c43)cc21 ; c1ccc(-c2ccc(N(c3ccccc3)c3c(-c4ccccc4)ccc4c3oc3c5ccccc5c5ccccc5c43)cc2)cc1 ; c1ccc(-c2ccc3c(oc4c5ccccc5c5ccccc5c34)c2N(c2ccccc2)c2cccc3ccccc23)cc1 ; c1ccc(-c2ccc3c(oc4c5ccccc5c5ccccc5c34)c2N(c2ccccc2)c2ccc3ccccc3c2)cc1]

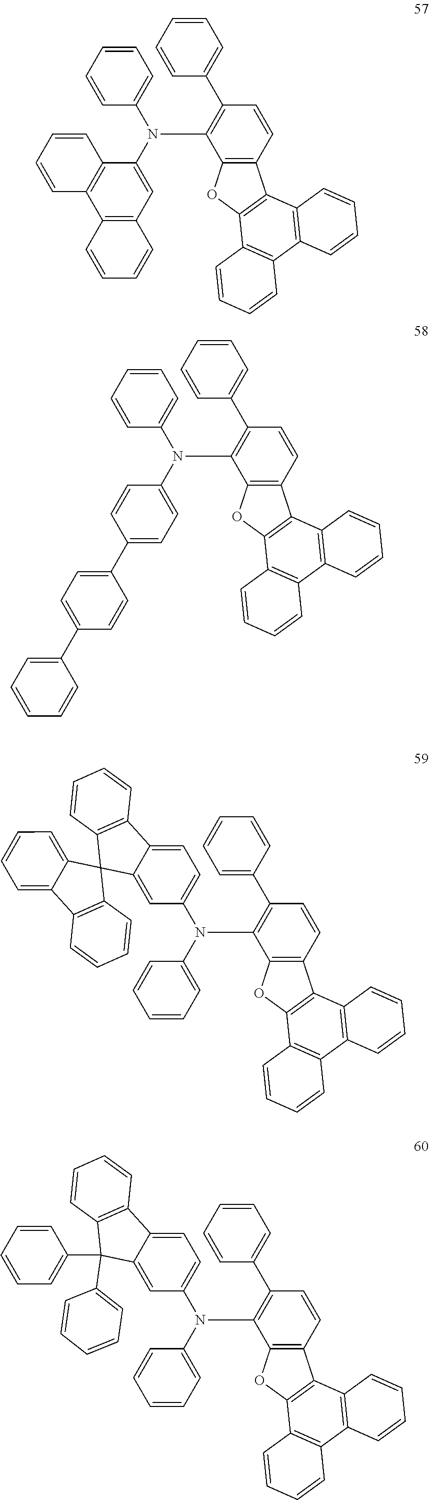

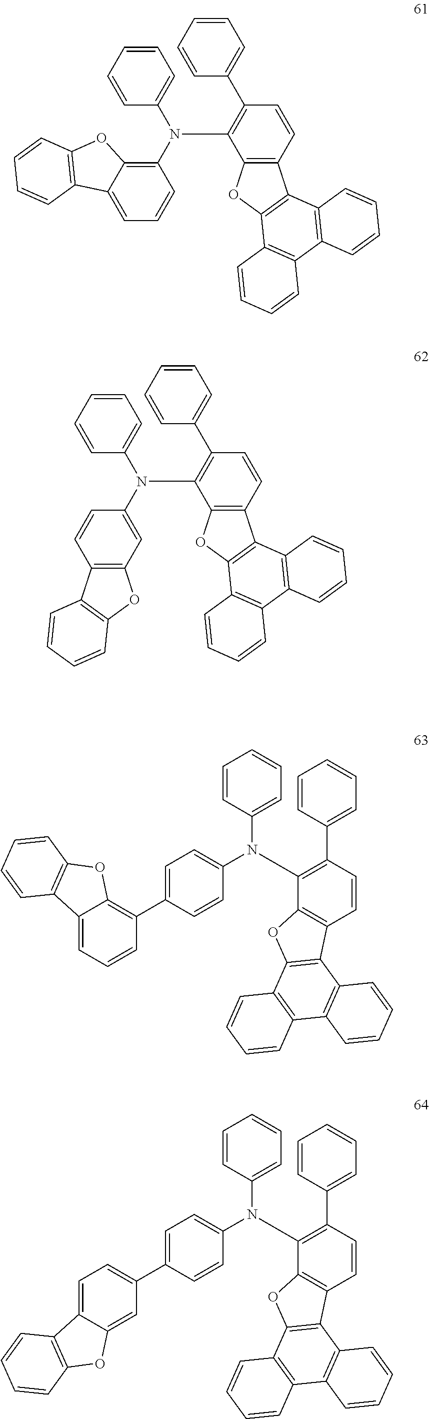 [SMILES: c1ccc(-c2ccc3c(oc4c5ccccc5c5ccccc5c34)c2N(c2ccccc2)c2cccc3c2oc2ccccc23)cc1 ; c1ccc(-c2ccc3c(oc4c5ccccc5c5ccccc5c34)c2N(c2ccccc2)c2ccc3c(c2)oc2ccccc23)cc1 ; c1ccc(-c2ccc3c(oc4c5ccccc5c5ccccc5c34)c2N(c2ccccc2)c2ccc(-c3cccc4c3oc3ccccc34)cc2)cc1 ; c1ccc(-c2ccc3c(oc4c5ccccc5c5ccccc5c34)c2N(c2ccccc2)c2ccc(-c3ccc4c(c3)oc3ccccc34)cc2)cc1]

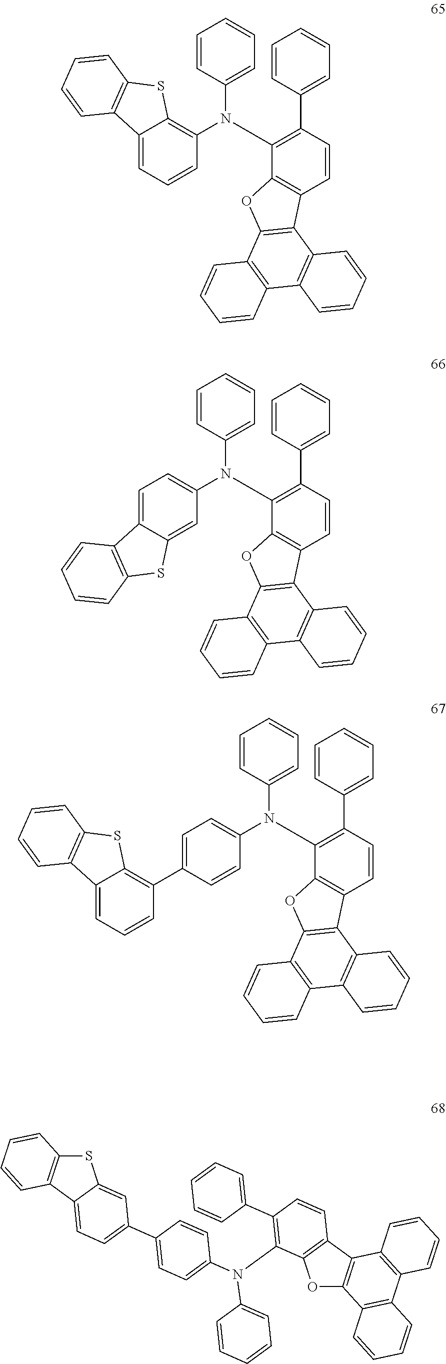 [SMILES: c1ccc(-c2ccc3c(oc4c5ccccc5c5ccccc5c34)c2N(c2ccccc2)c2cccc3c2sc2ccccc23)cc1 ; c1ccc(-c2ccc3c(oc4c5ccccc5c5ccccc5c34)c2N(c2ccccc2)c2ccc3c(c2)sc2ccccc23)cc1 ; c1ccc(-c2ccc3c(oc4c5ccccc5c5ccccc5c34)c2N(c2ccccc2)c2ccc(-c3cccc4c3sc3ccccc34)cc2)cc1 ; c1ccc(-c2ccc3c(oc4c5ccccc5c5ccccc5c34)c2N(c2ccccc2)c2ccc(-c3ccc4c(c3)sc3ccccc34)cc2)cc1]

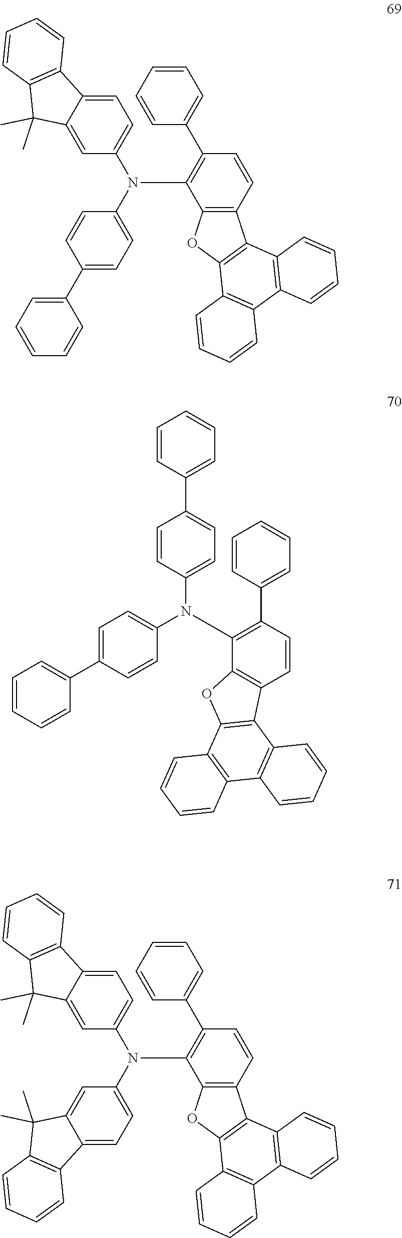 [SMILES: CC1(C)c2ccccc2-c2ccc(N(c3ccc(-c4ccccc4)cc3)c3c(-c4ccccc4)ccc4c3oc3c5ccccc5c5ccccc5c43)cc21 ; c1ccc(-c2ccc(N(c3ccc(-c4ccccc4)cc3)c3c(-c4ccccc4)ccc4c3oc3c5ccccc5c5ccccc5c43)cc2)cc1 ; CC1(C)c2ccccc2-c2ccc(N(c3ccc4c(c3)C(C)(C)c3ccccc3-4)c3c(-c4ccccc4)ccc4c3oc3c5ccccc5c5ccccc5c43)cc21]

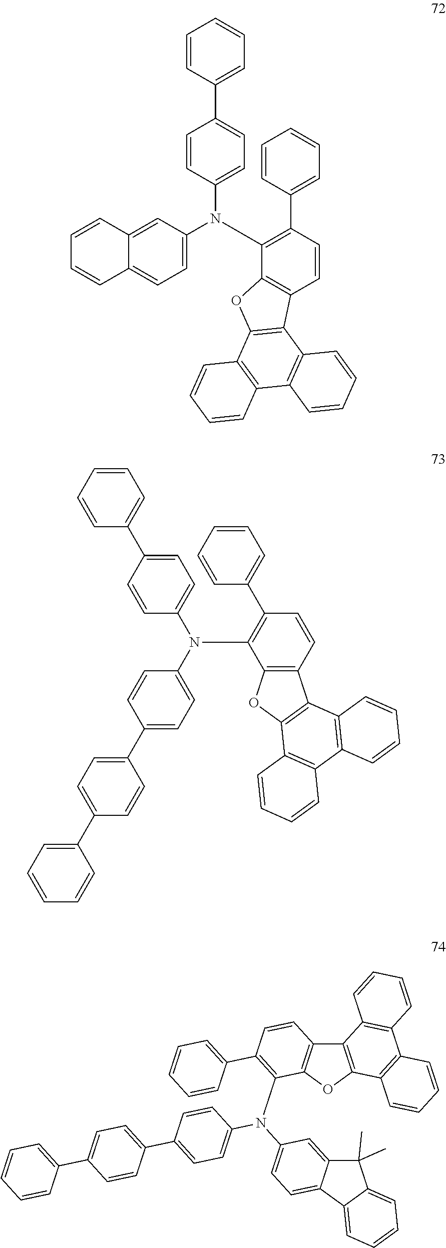 [SMILES: c1ccc(-c2ccc(N(c3ccc4ccccc4c3)c3c(-c4ccccc4)ccc4c3oc3c5ccccc5c5ccccc5c43)cc2)cc1 ; c1ccc(-c2ccc(-c3ccc(N(c4ccc(-c5ccccc5)cc4)c4c(-c5ccccc5)ccc5c4oc4c6ccccc6c6ccccc6c54)cc3)cc2)cc1 ; CC1(C)c2ccccc2-c2ccc(N(c3ccc(-c4ccc(-c5ccccc5)cc4)cc3)c3c(-c4ccccc4)ccc4c3oc3c5ccccc5c5ccccc5c43)cc21]

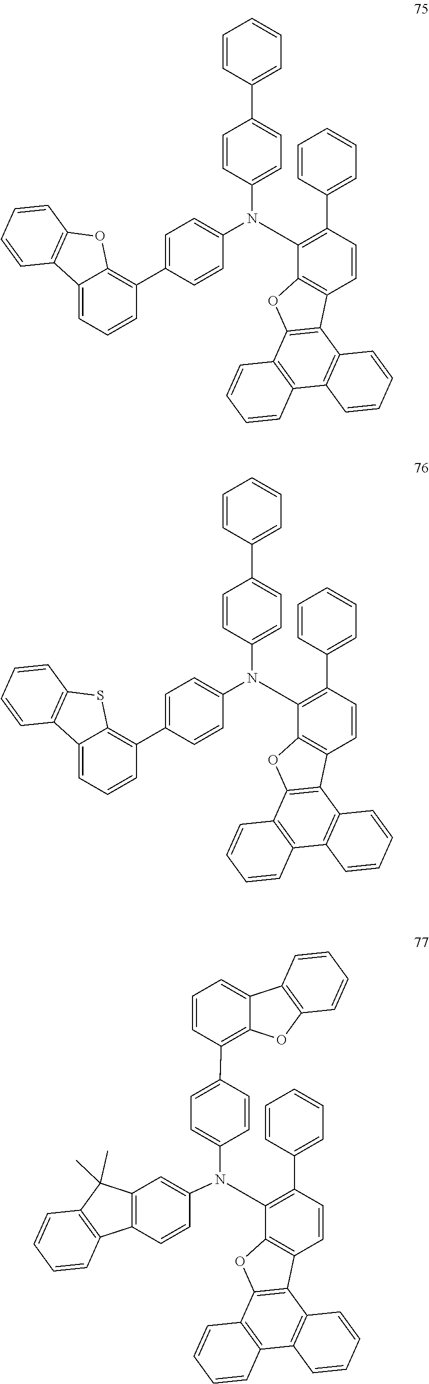 [SMILES: c1ccc(-c2ccc(N(c3ccc(-c4cccc5c4oc4ccccc45)cc3)c3c(-c4ccccc4)ccc4c3oc3c5ccccc5c5ccccc5c43)cc2)cc1 ; c1ccc(-c2ccc(N(c3ccc(-c4cccc5c4sc4ccccc45)cc3)c3c(-c4ccccc4)ccc4c3oc3c5ccccc5c5ccccc5c43)cc2)cc1 ; CC1(C)c2ccccc2-c2ccc(N(c3ccc(-c4cccc5c4oc4ccccc45)cc3)c3c(-c4ccccc4)ccc4c3oc3c5ccccc5c5ccccc5c43)cc21]

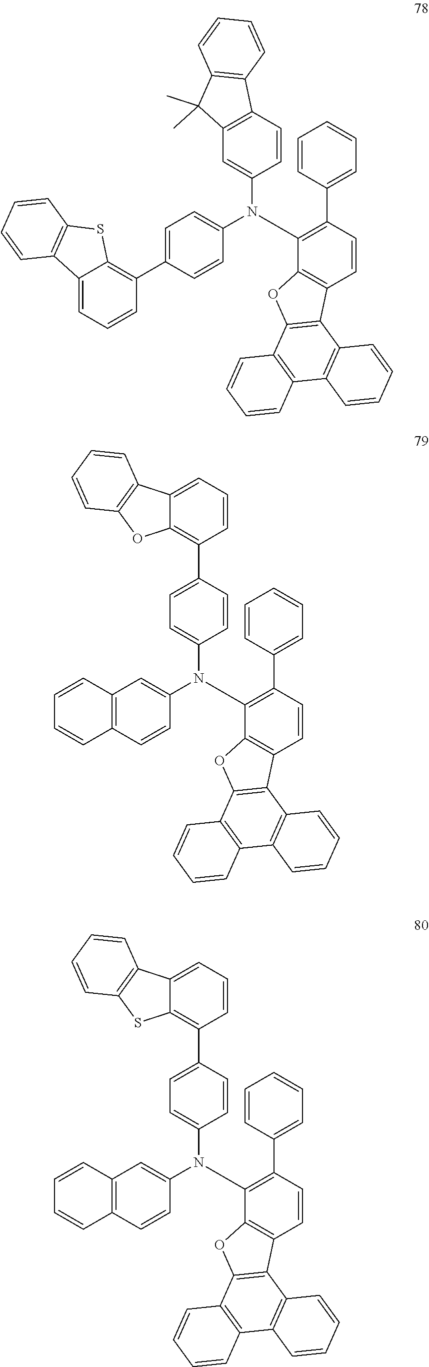 [SMILES: CC1(C)c2ccccc2-c2ccc(N(c3ccc(-c4cccc5c4sc4ccccc45)cc3)c3c(-c4ccccc4)ccc4c3oc3c5ccccc5c5ccccc5c43)cc21 ; c1ccc(-c2ccc3c(oc4c5ccccc5c5ccccc5c34)c2N(c2ccc(-c3cccc4c3oc3ccccc34)cc2)c2ccc3ccccc3c2)cc1 ; c1ccc(-c2ccc3c(oc4c5ccccc5c5ccccc5c34)c2N(c2ccc(-c3cccc4c3sc3ccccc34)cc2)c2ccc3ccccc3c2)cc1]

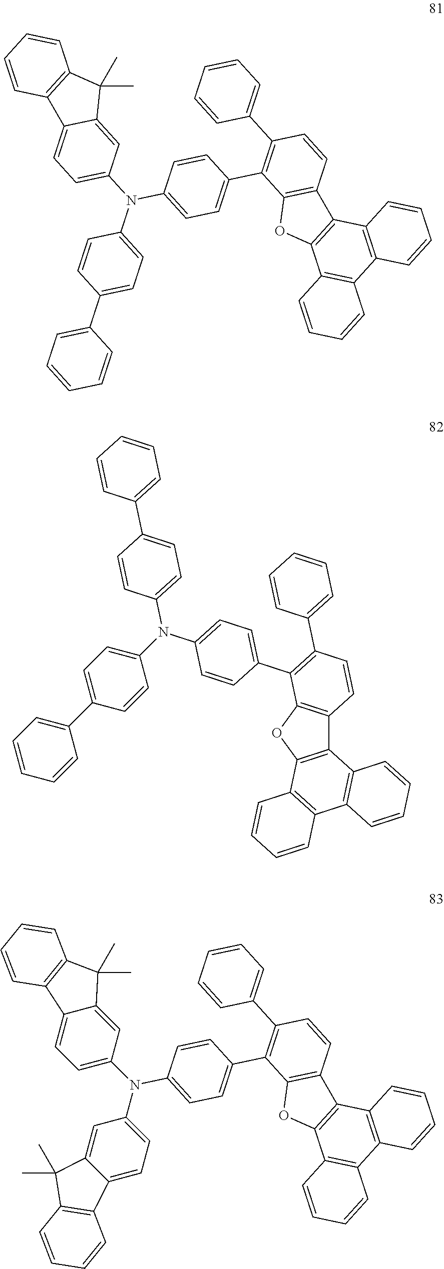 [SMILES: CC1(C)c2ccccc2-c2ccc(N(c3ccc(-c4ccccc4)cc3)c3ccc(-c4c(-c5ccccc5)ccc5c4oc4c6ccccc6c6ccccc6c54)cc3)cc21 ; c1ccc(-c2ccc(N(c3ccc(-c4ccccc4)cc3)c3ccc(-c4c(-c5ccccc5)ccc5c4oc4c6ccccc6c6ccccc6c54)cc3)cc2)cc1 ; CC1(C)c2ccccc2-c2ccc(N(c3ccc(-c4c(-c5ccccc5)ccc5c4oc4c6ccccc6c6ccccc6c54)cc3)c3ccc4c(c3)C(C)(C)c3ccccc3-4)cc21]

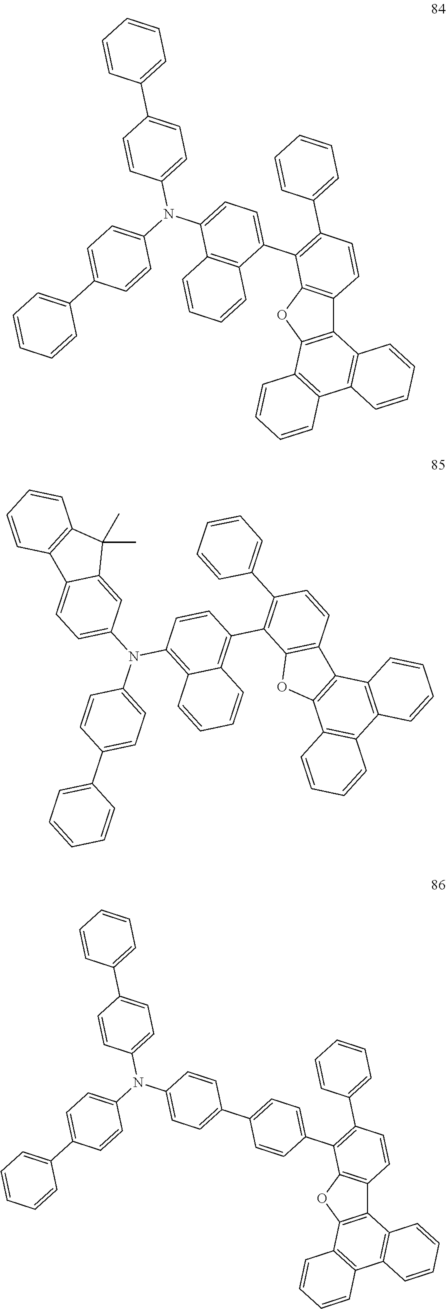 [SMILES: c1ccc(-c2ccc(N(c3ccc(-c4ccccc4)cc3)c3ccc(-c4c(-c5ccccc5)ccc5c4oc4c6ccccc6c6ccccc6c54)c4ccccc34)cc2)cc1 ; CC1(C)c2ccccc2-c2ccc(N(c3ccc(-c4ccccc4)cc3)c3ccc(-c4c(-c5ccccc5)ccc5c4oc4c6ccccc6c6ccccc6c54)c4ccccc34)cc21 ; c1ccc(-c2ccc(N(c3ccc(-c4ccccc4)cc3)c3ccc(-c4ccc(-c5c(-c6ccccc6)ccc6c5oc5c7ccccc7c7ccccc7c65)cc4)cc3)cc2)cc1]

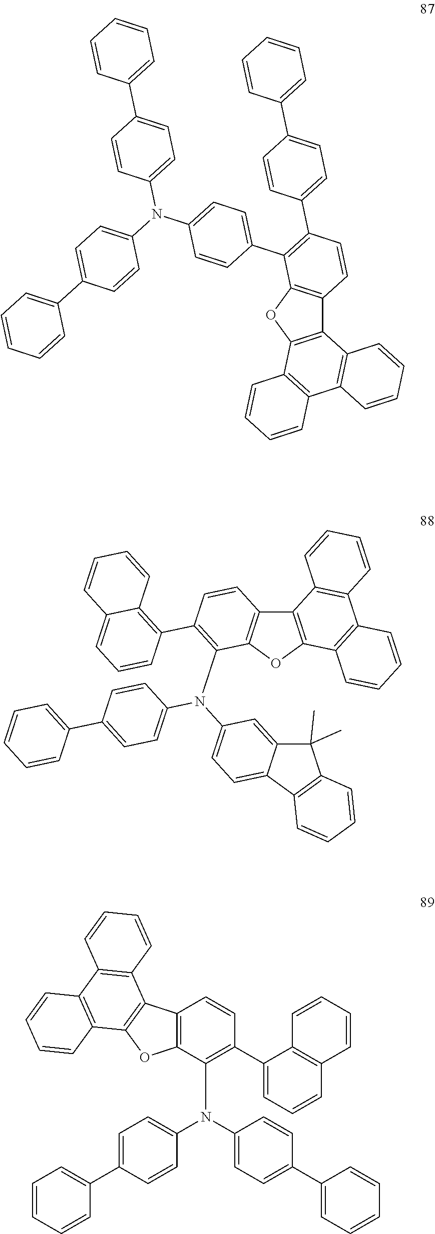 [SMILES: c1ccc(-c2ccc(-c3ccc4c(oc5c6ccccc6c6ccccc6c45)c3-c3ccc(N(c4ccc(-c5ccccc5)cc4)c4ccc(-c5ccccc5)cc4)cc3)cc2)cc1 ; CC1(C)c2ccccc2-c2ccc(N(c3ccc(-c4ccccc4)cc3)c3c(-c4cccc5ccccc45)ccc4c3oc3c5ccccc5c5ccccc5c43)cc21 ; c1ccc(-c2ccc(N(c3ccc(-c4ccccc4)cc3)c3c(-c4cccc5ccccc45)ccc4c3oc3c5ccccc5c5ccccc5c43)cc2)cc1]

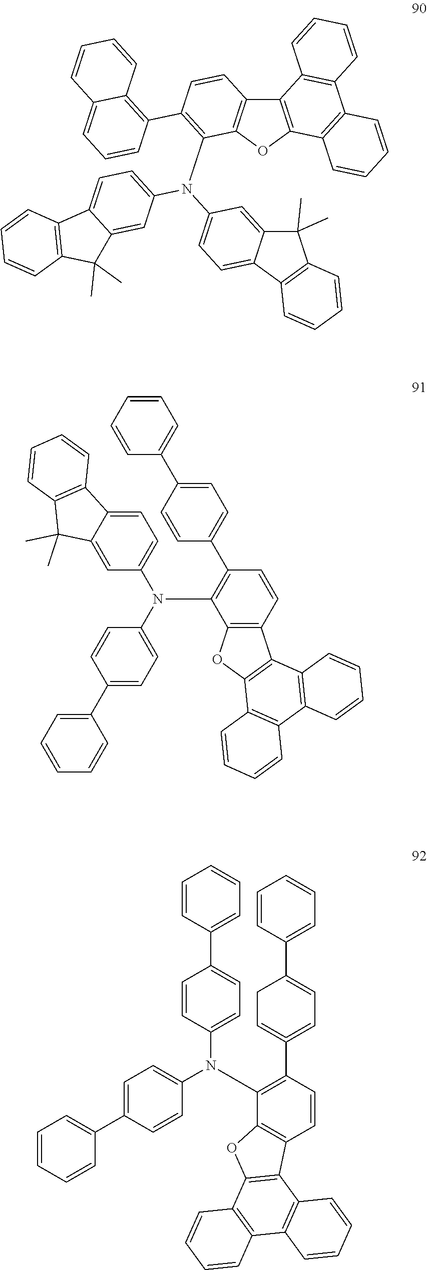

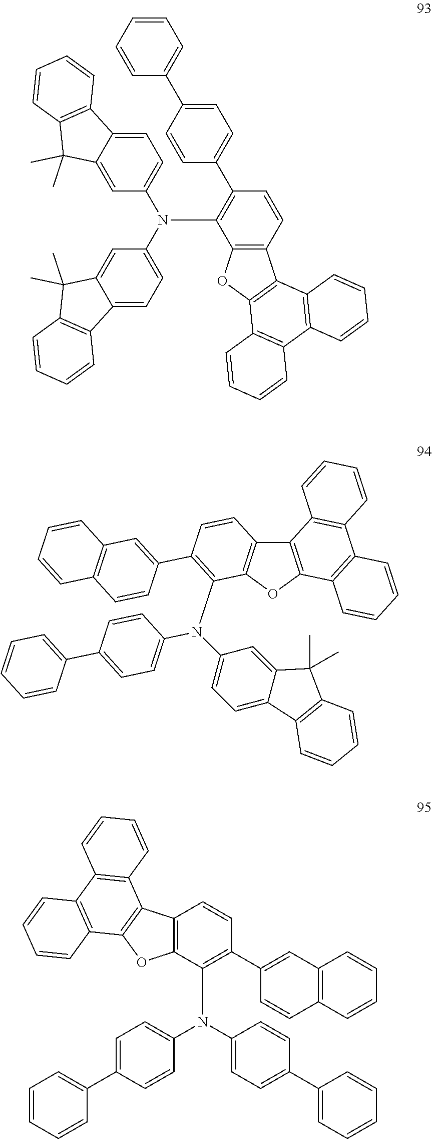 [SMILES: CC1(C)c2ccccc2-c2ccc(N(c3ccc4c(c3)C(C)(C)c3ccccc3-4)c3c(-c4ccc(-c5ccccc5)cc4)ccc4c3oc3c5ccccc5c5ccccc5c43)cc21 ; CC1(C)c2ccccc2-c2ccc(N(c3ccc(-c4ccccc4)cc3)c3c(-c4ccc5ccccc5c4)ccc4c3oc3c5ccccc5c5ccccc5c43)cc21 ; c1ccc(-c2ccc(N(c3ccc(-c4ccccc4)cc3)c3c(-c4ccc5ccccc5c4)ccc4c3oc3c5ccccc5c5ccccc5c43)cc2)cc1]

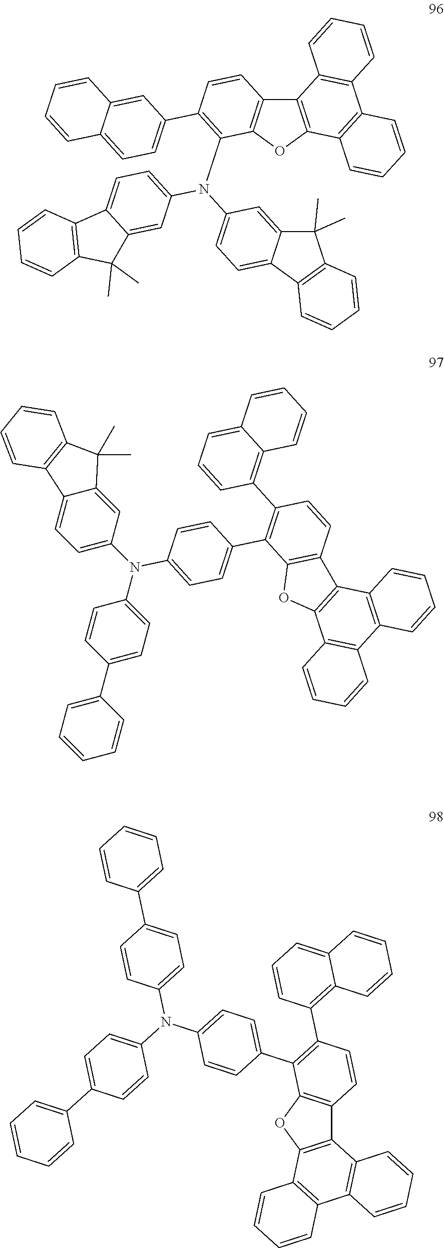 [SMILES: CC1(C)c2ccccc2-c2ccc(N(c3ccc4c(c3)C(C)(C)c3ccccc3-4)c3c(-c4ccc5ccccc5c4)ccc4c3oc3c5ccccc5c5ccccc5c43)cc21 ; CC1(C)c2ccccc2-c2ccc(N(c3ccc(-c4ccccc4)cc3)c3ccc(-c4c(-c5cccc6ccccc56)ccc5c4oc4c6ccccc6c6ccccc6c54)cc3)cc21 ; c1ccc(-c2ccc(N(c3ccc(-c4ccccc4)cc3)c3ccc(-c4c(-c5cccc6ccccc56)ccc5c4oc4c6ccccc6c6ccccc6c54)cc3)cc2)cc1]

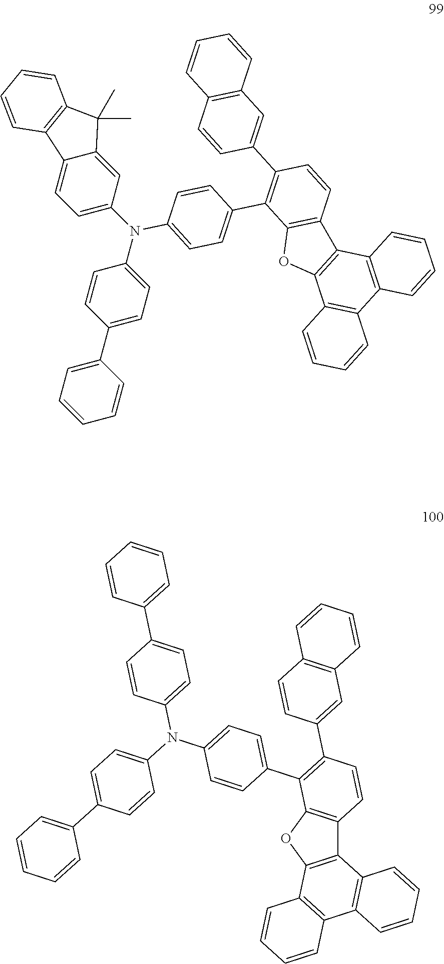 [SMILES: CC1(C)c2ccccc2-c2ccc(N(c3ccc(-c4ccccc4)cc3)c3ccc(-c4c(-c5ccc6ccccc6c5)ccc5c4oc4c6ccccc6c6ccccc6c54)cc3)cc21 ; c1ccc(-c2ccc(N(c3ccc(-c4ccccc4)cc3)c3ccc(-c4c(-c5ccc6ccccc6c5)ccc5c4oc4c6ccccc6c6ccccc6c54)cc3)cc2)cc1]

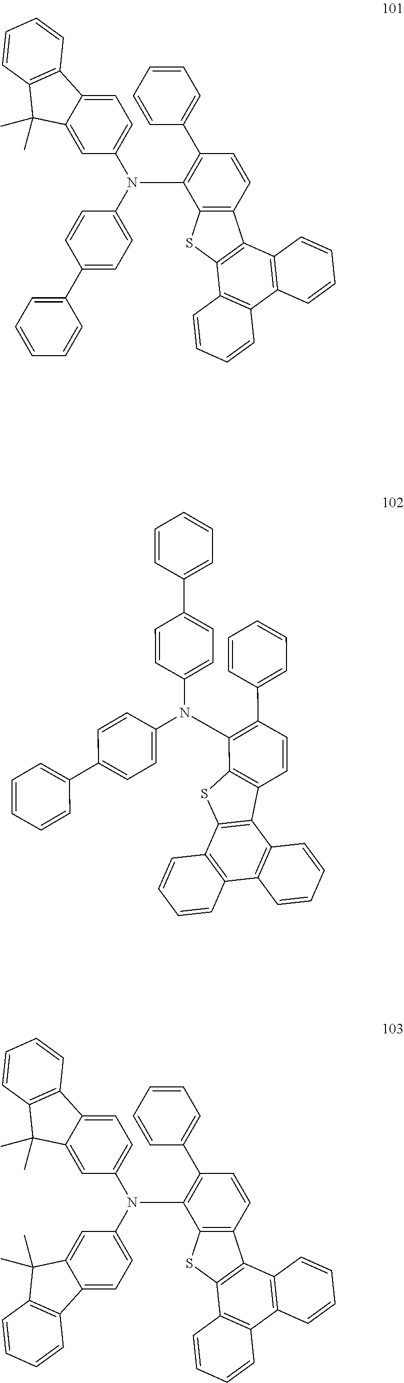 [SMILES: CC1(C)c2ccccc2-c2ccc(N(c3ccc(-c4ccccc4)cc3)c3c(-c4ccccc4)ccc4c3sc3c5ccccc5c5ccccc5c43)cc21 ; c1ccc(-c2ccc(N(c3ccc(-c4ccccc4)cc3)c3c(-c4ccccc4)ccc4c3sc3c5ccccc5c5ccccc5c43)cc2)cc1 ; CC1(C)c2ccccc2-c2ccc(N(c3ccc4c(c3)C(C)(C)c3ccccc3-4)c3c(-c4ccccc4)ccc4c3sc3c5ccccc5c5ccccc5c43)cc21]

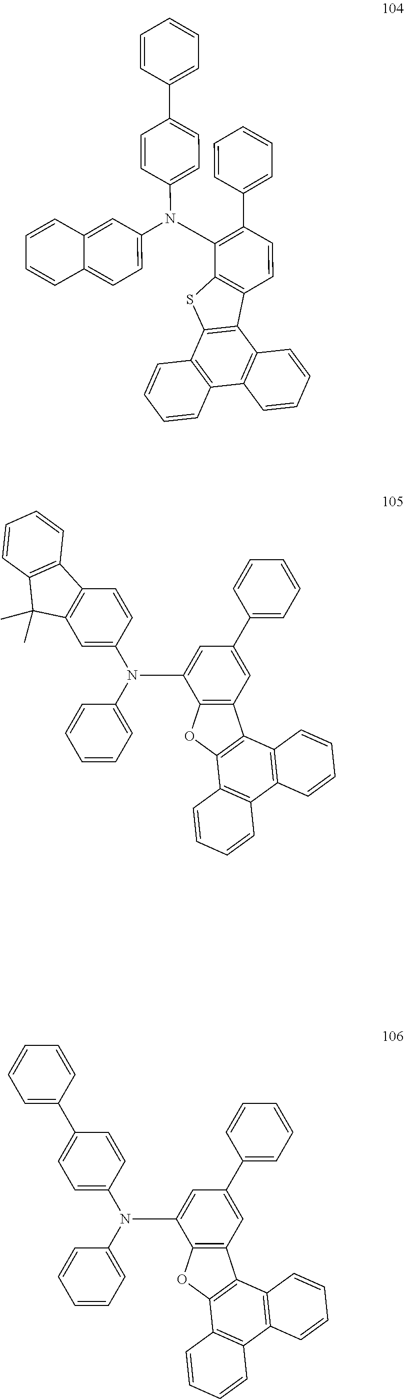 [SMILES: c1ccc(-c2ccc(N(c3ccc4ccccc4c3)c3c(-c4ccccc4)ccc4c3sc3c5ccccc5c5ccccc5c43)cc2)cc1 ; CC1(C)c2ccccc2-c2ccc(N(c3ccccc3)c3cc(-c4ccccc4)cc4c3oc3c5ccccc5c5ccccc5c43)cc21 ; c1ccc(-c2ccc(N(c3ccccc3)c3cc(-c4ccccc4)cc4c3oc3c5ccccc5c5ccccc5c43)cc2)cc1]

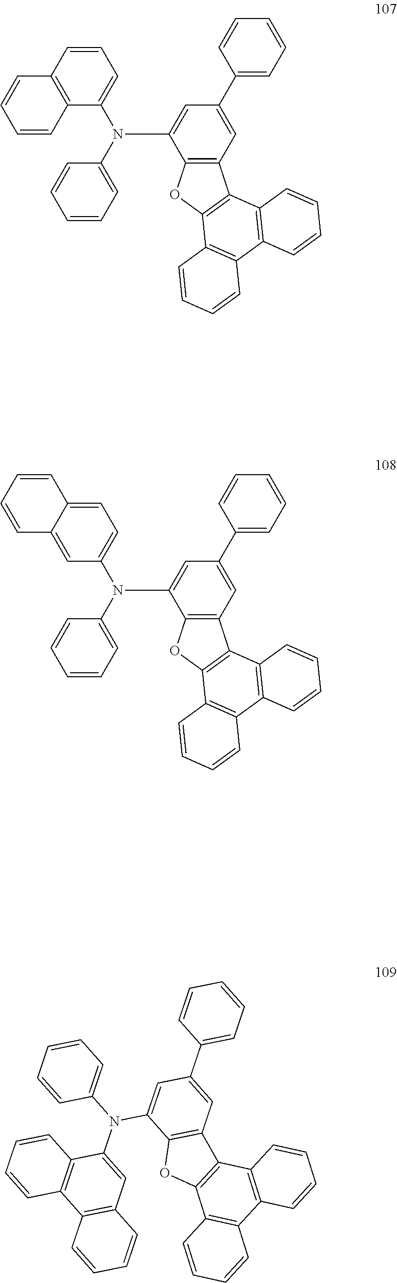 [SMILES: c1ccc(-c2cc(N(c3ccccc3)c3cccc4ccccc34)c3oc4c5ccccc5c5ccccc5c4c3c2)cc1 ; c1ccc(-c2cc(N(c3ccccc3)c3ccc4ccccc4c3)c3oc4c5ccccc5c5ccccc5c4c3c2)cc1 ; c1ccc(-c2cc(N(c3ccccc3)c3cc4ccccc4c4ccccc34)c3oc4c5ccccc5c5ccccc5c4c3c2)cc1]

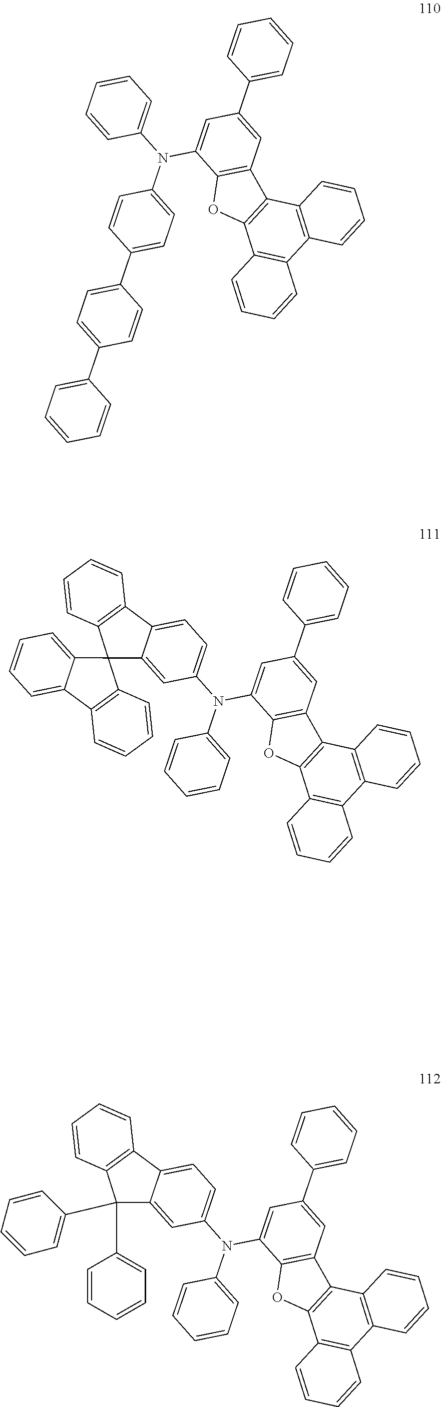 [SMILES: c1ccc(-c2ccc(-c3ccc(N(c4ccccc4)c4cc(-c5ccccc5)cc5c4oc4c6ccccc6c6ccccc6c54)cc3)cc2)cc1 ; c1ccc(-c2cc(N(c3ccccc3)c3ccc4c(c3)C3(c5ccccc5-c5ccccc53)c3ccccc3-4)c3oc4c5ccccc5c5ccccc5c4c3c2)cc1 ; c1ccc(-c2cc(N(c3ccccc3)c3ccc4c(c3)C(c3ccccc3)(c3ccccc3)c3ccccc3-4)c3oc4c5ccccc5c5ccccc5c4c3c2)cc1]

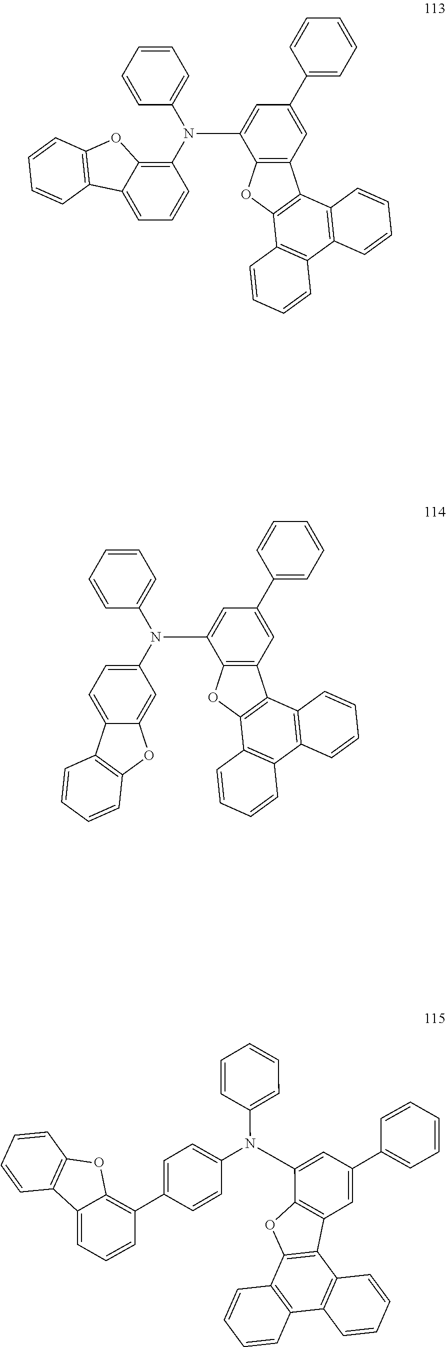 [SMILES: c1ccc(-c2cc(N(c3ccccc3)c3cccc4c3oc3ccccc34)c3oc4c5ccccc5c5ccccc5c4c3c2)cc1 ; c1ccc(-c2cc(N(c3ccccc3)c3ccc4c(c3)oc3ccccc34)c3oc4c5ccccc5c5ccccc5c4c3c2)cc1 ; c1ccc(-c2cc(N(c3ccccc3)c3ccc(-c4cccc5c4oc4ccccc45)cc3)c3oc4c5ccccc5c5ccccc5c4c3c2)cc1]

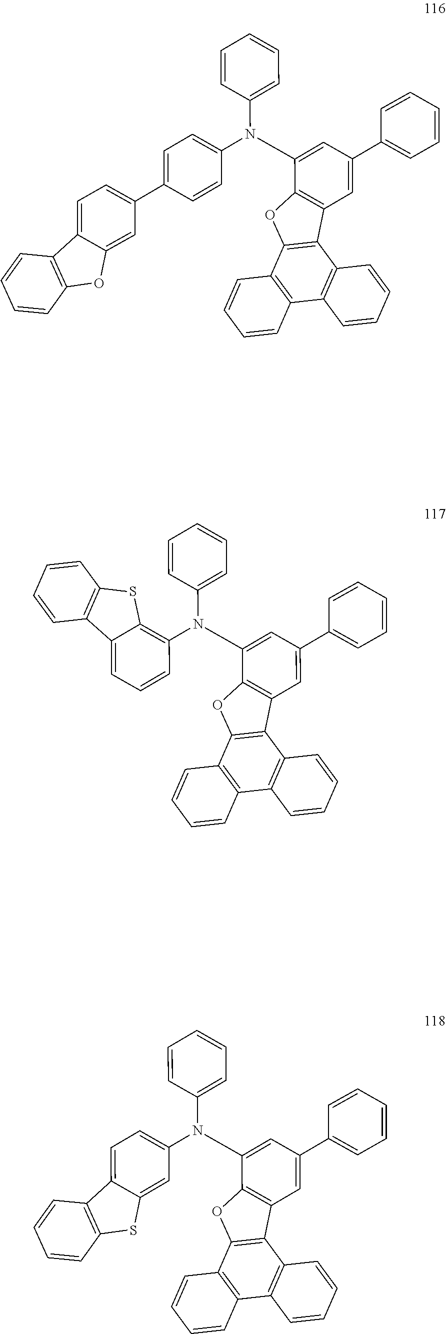 [SMILES: c1ccc(-c2cc(N(c3ccccc3)c3ccc(-c4ccc5c(c4)oc4ccccc45)cc3)c3oc4c5ccccc5c5ccccc5c4c3c2)cc1 ; c1ccc(-c2cc(N(c3ccccc3)c3cccc4c3sc3ccccc34)c3oc4c5ccccc5c5ccccc5c4c3c2)cc1 ; c1ccc(-c2cc(N(c3ccccc3)c3ccc4c(c3)sc3ccccc34)c3oc4c5ccccc5c5ccccc5c4c3c2)cc1]

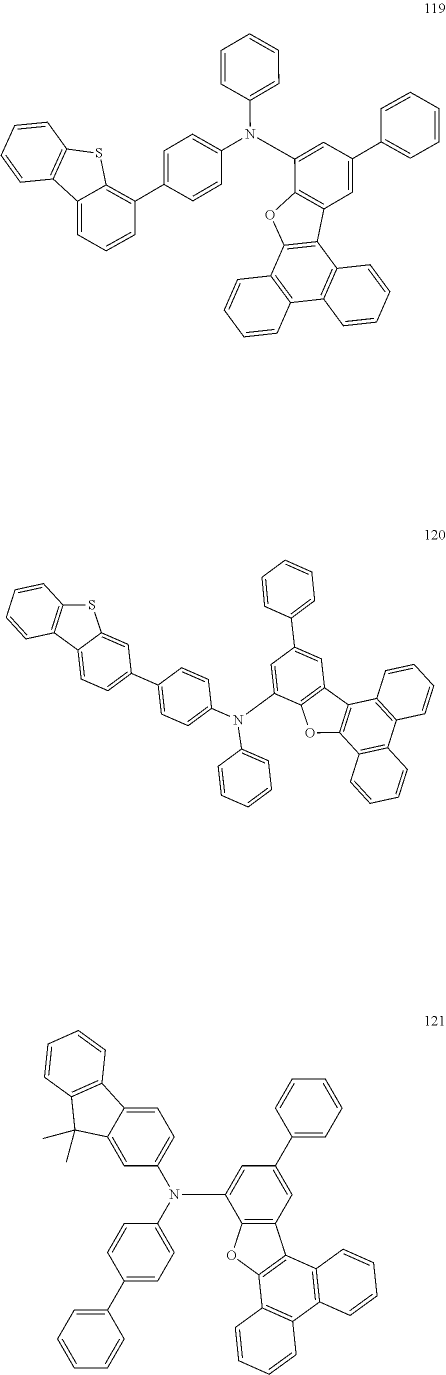 [SMILES: c1ccc(-c2cc(N(c3ccccc3)c3ccc(-c4cccc5c4sc4ccccc45)cc3)c3oc4c5ccccc5c5ccccc5c4c3c2)cc1 ; c1ccc(-c2cc(N(c3ccccc3)c3ccc(-c4ccc5c(c4)sc4ccccc45)cc3)c3oc4c5ccccc5c5ccccc5c4c3c2)cc1 ; CC1(C)c2ccccc2-c2ccc(N(c3ccc(-c4ccccc4)cc3)c3cc(-c4ccccc4)cc4c3oc3c5ccccc5c5ccccc5c43)cc21]

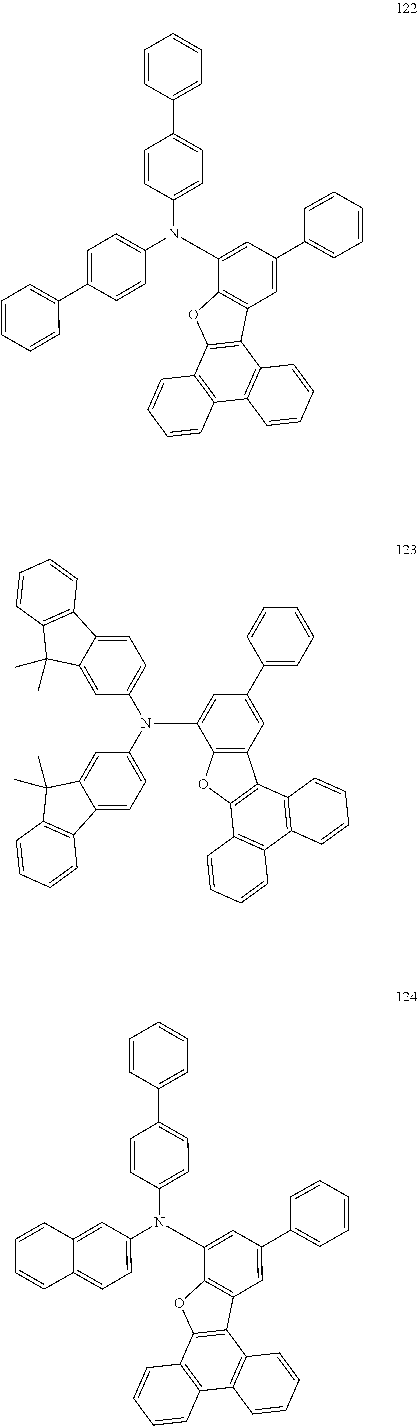 [SMILES: c1ccc(-c2ccc(N(c3ccc(-c4ccccc4)cc3)c3cc(-c4ccccc4)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4ccccc4)cc4c3oc3c5ccccc5c5ccccc5c43)cc21 ; c1ccc(-c2ccc(N(c3ccc4ccccc4c3)c3cc(-c4ccccc4)cc4c3oc3c5ccccc5c5ccccc5c43)cc2)cc1]

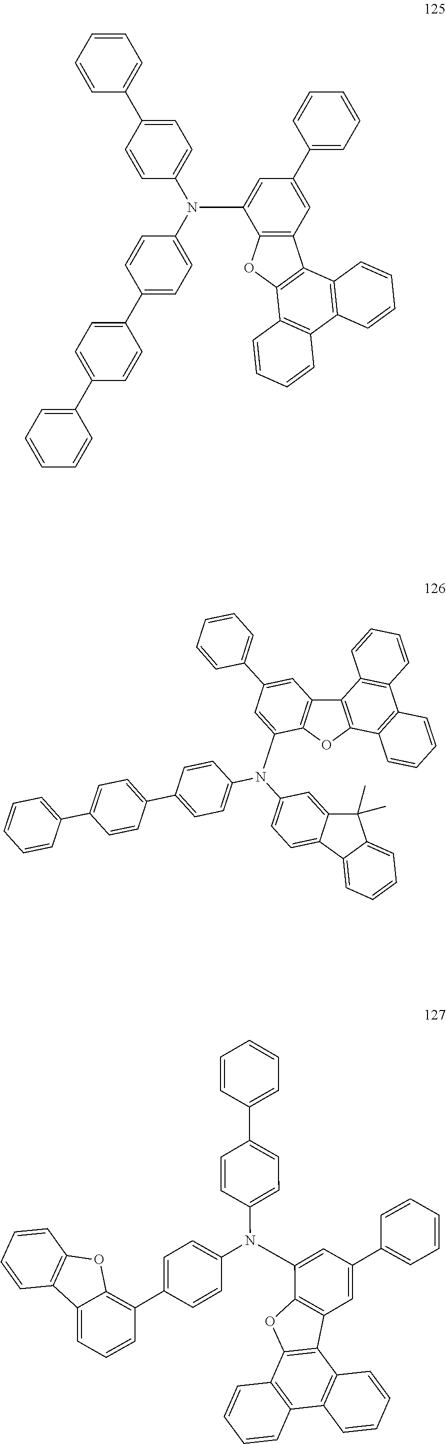 [SMILES: c1ccc(-c2ccc(-c3ccc(N(c4ccc(-c5ccccc5)cc4)c4cc(-c5ccccc5)cc5c4oc4c6ccccc6c6ccccc6c54)cc3)cc2)cc1 ; CC1(C)c2ccccc2-c2ccc(N(c3ccc(-c4ccc(-c5ccccc5)cc4)cc3)c3cc(-c4ccccc4)cc4c3oc3c5ccccc5c5ccccc5c43)cc21 ; c1ccc(-c2ccc(N(c3ccc(-c4cccc5c4oc4ccccc45)cc3)c3cc(-c4ccccc4)cc4c3oc3c5ccccc5c5ccccc5c43)cc2)cc1]

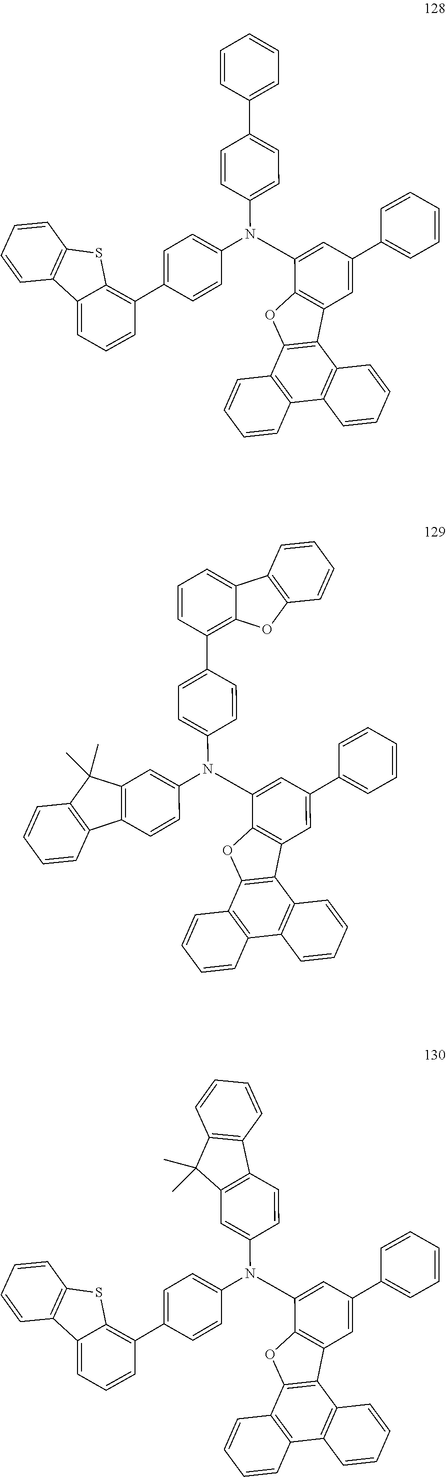 [SMILES: c1ccc(-c2ccc(N(c3ccc(-c4cccc5c4sc4ccccc45)cc3)c3cc(-c4ccccc4)cc4c3oc3c5ccccc5c5ccccc5c43)cc2)cc1 ; CC1(C)c2ccccc2-c2ccc(N(c3ccc(-c4cccc5c4oc4ccccc45)cc3)c3cc(-c4ccccc4)cc4c3oc3c5ccccc5c5ccccc5c43)cc21 ; CC1(C)c2ccccc2-c2ccc(N(c3ccc(-c4cccc5c4sc4ccccc45)cc3)c3cc(-c4ccccc4)cc4c3oc3c5ccccc5c5ccccc5c43)cc21]

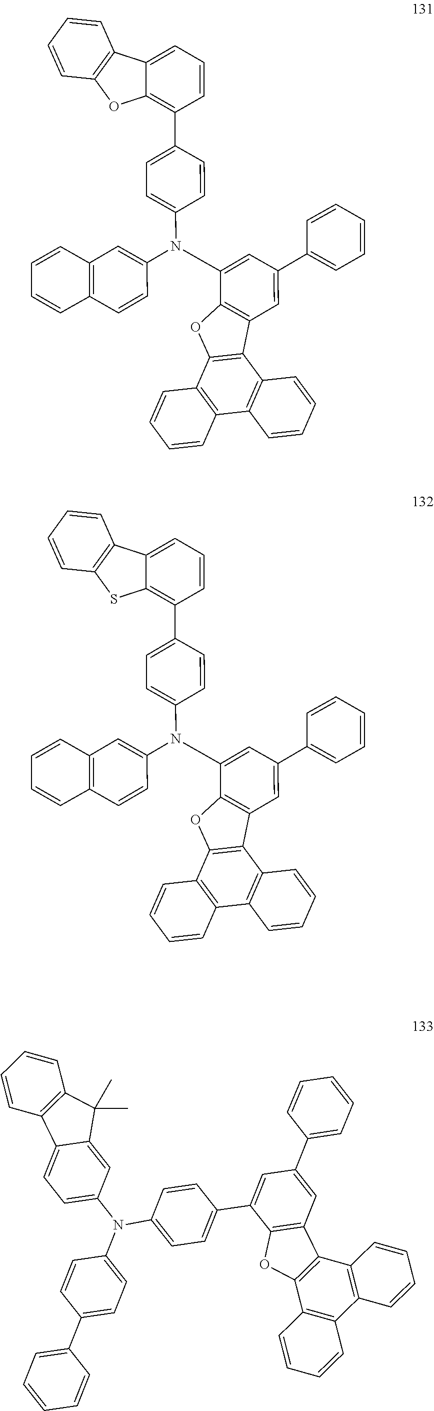 [SMILES: c1ccc(-c2cc(N(c3ccc(-c4cccc5c4oc4ccccc45)cc3)c3ccc4ccccc4c3)c3oc4c5ccccc5c5ccccc5c4c3c2)cc1 ; c1ccc(-c2cc(N(c3ccc(-c4cccc5c4sc4ccccc45)cc3)c3ccc4ccccc4c3)c3oc4c5ccccc5c5ccccc5c4c3c2)cc1 ; CC1(C)c2ccccc2-c2ccc(N(c3ccc(-c4ccccc4)cc3)c3ccc(-c4cc(-c5ccccc5)cc5c4oc4c6ccccc6c6ccccc6c54)cc3)cc21]

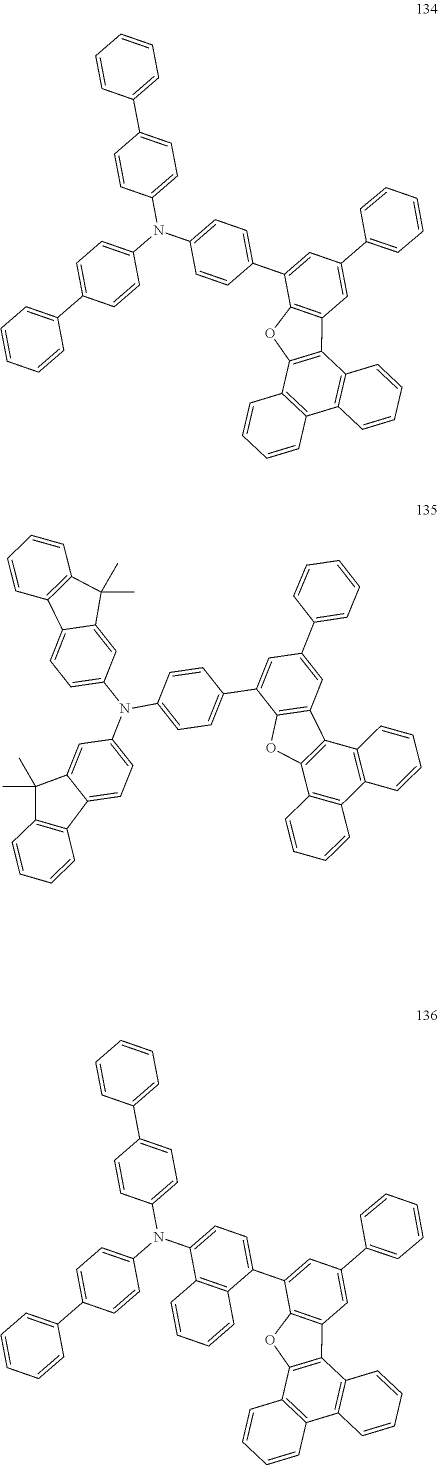 [SMILES: c1ccc(-c2ccc(N(c3ccc(-c4ccccc4)cc3)c3ccc(-c4cc(-c5ccccc5)cc5c4oc4c6ccccc6c6ccccc6c54)cc3)cc2)cc1 ; CC1(C)c2ccccc2-c2ccc(N(c3ccc(-c4cc(-c5ccccc5)cc5c4oc4c6ccccc6c6ccccc6c54)cc3)c3ccc4c(c3)C(C)(C)c3ccccc3-4)cc21 ; c1ccc(-c2ccc(N(c3ccc(-c4ccccc4)cc3)c3ccc(-c4cc(-c5ccccc5)cc5c4oc4c6ccccc6c6ccccc6c54)c4ccccc34)cc2)cc1]

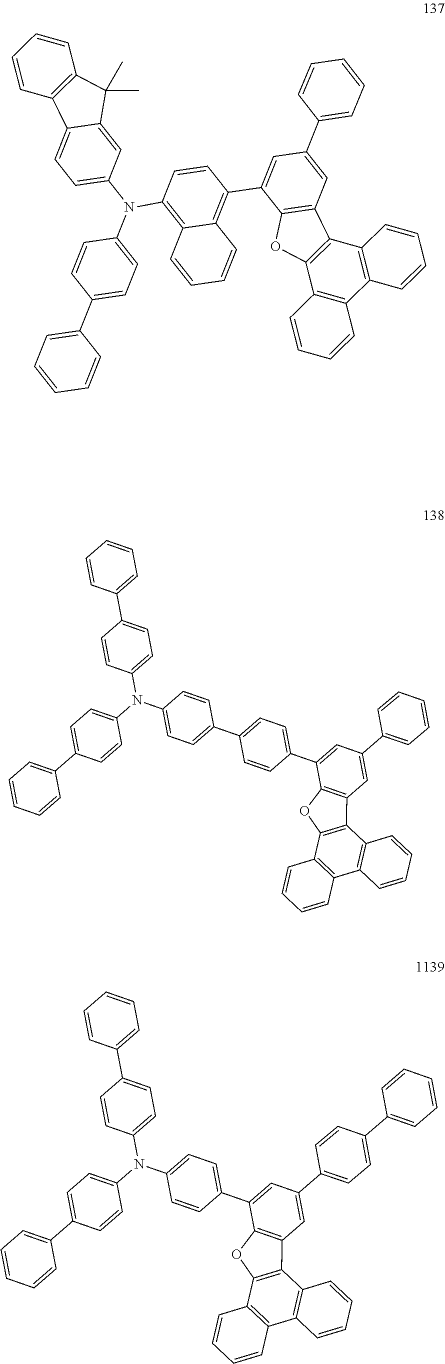 [SMILES: CC1(C)c2ccccc2-c2ccc(N(c3ccc(-c4ccccc4)cc3)c3ccc(-c4cc(-c5ccccc5)cc5c4oc4c6ccccc6c6ccccc6c54)c4ccccc34)cc21 ; c1ccc(-c2ccc(N(c3ccc(-c4ccccc4)cc3)c3ccc(-c4ccc(-c5cc(-c6ccccc6)cc6c5oc5c7ccccc7c7ccccc7c65)cc4)cc3)cc2)cc1 ; c1ccc(-c2ccc(-c3cc(-c4ccc(N(c5ccc(-c6ccccc6)cc5)c5ccc(-c6ccccc6)cc5)cc4)c4oc5c6ccccc6c6ccccc6c5c4c3)cc2)cc1]

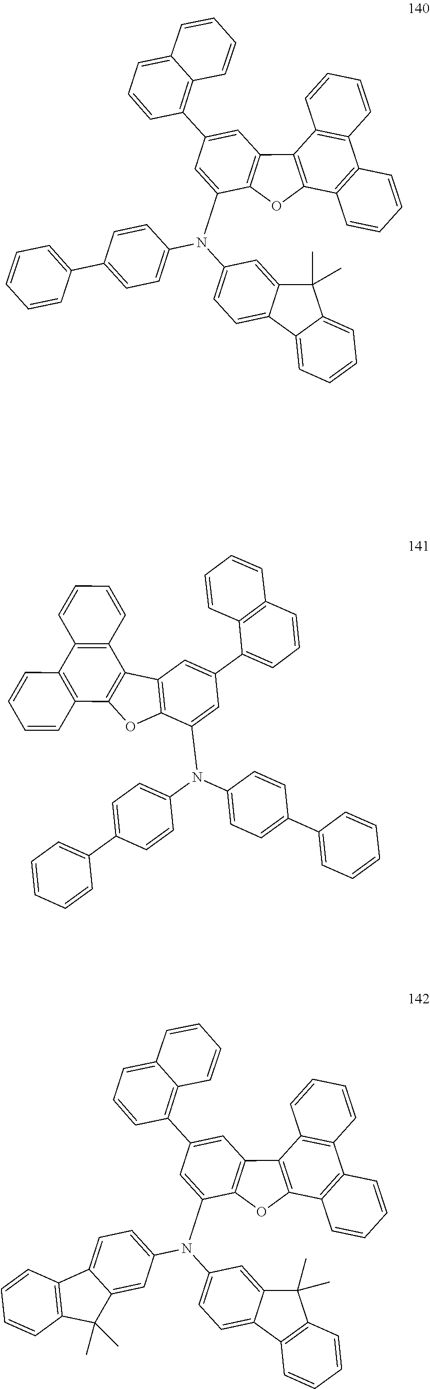 [SMILES: CC1(C)c2ccccc2-c2ccc(N(c3ccc(-c4ccccc4)cc3)c3cc(-c4cccc5ccccc45)cc4c3oc3c5ccccc5c5ccccc5c43)cc21 ; c1ccc(-c2ccc(N(c3ccc(-c4ccccc4)cc3)c3cc(-c4cccc5ccccc45)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4cccc5ccccc45)cc4c3oc3c5ccccc5c5ccccc5c43)cc21]

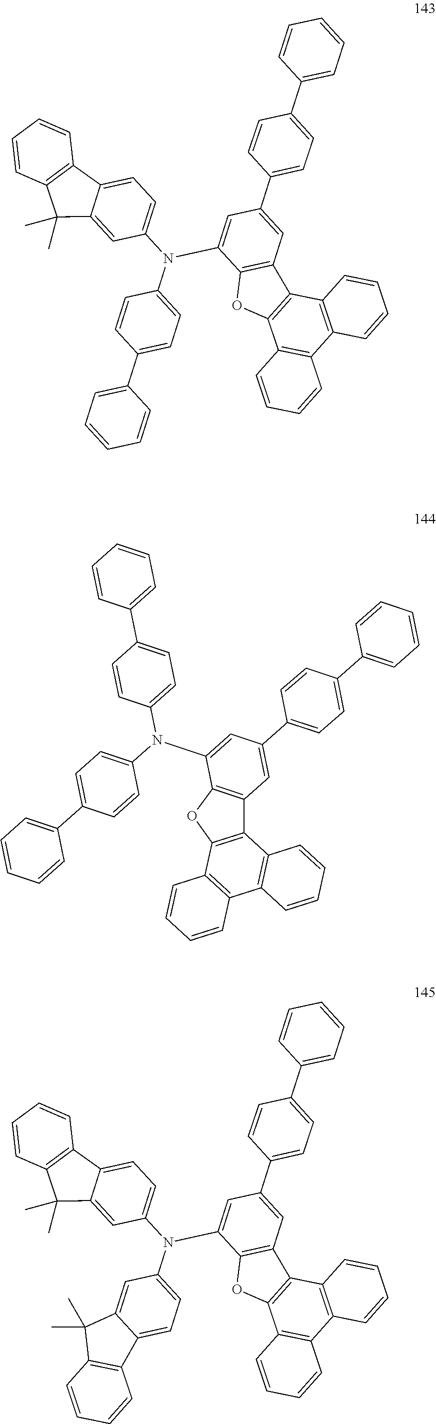 [SMILES: CC1(C)c2ccccc2-c2ccc(N(c3ccc(-c4ccccc4)cc3)c3cc(-c4ccc(-c5ccccc5)cc4)cc4c3oc3c5ccccc5c5ccccc5c43)cc21 ; c1ccc(-c2ccc(-c3cc(N(c4ccc(-c5ccccc5)cc4)c4ccc(-c5ccccc5)cc4)c4oc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccc(-c5ccccc5)cc4)cc4c3oc3c5ccccc5c5ccccc5c43)cc21]

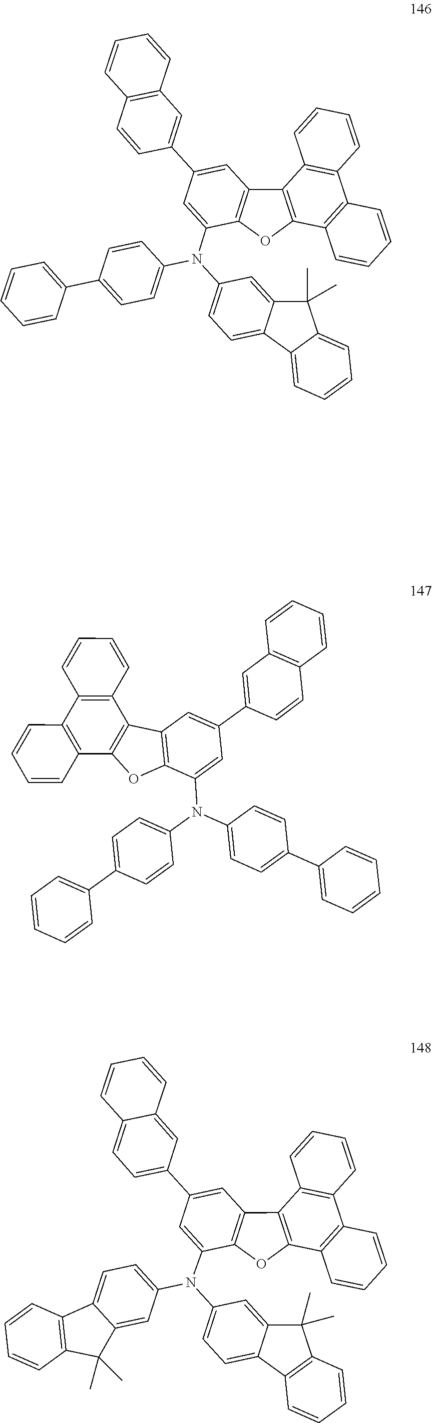 [SMILES: CC1(C)c2ccccc2-c2ccc(N(c3ccc(-c4ccccc4)cc3)c3cc(-c4ccc5ccccc5c4)cc4c3oc3c5ccccc5c5ccccc5c43)cc21 ; c1ccc(-c2ccc(N(c3ccc(-c4ccccc4)cc3)c3cc(-c4ccc5ccccc5c4)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4ccc5ccccc5c4)cc4c3oc3c5ccccc5c5ccccc5c43)cc21]

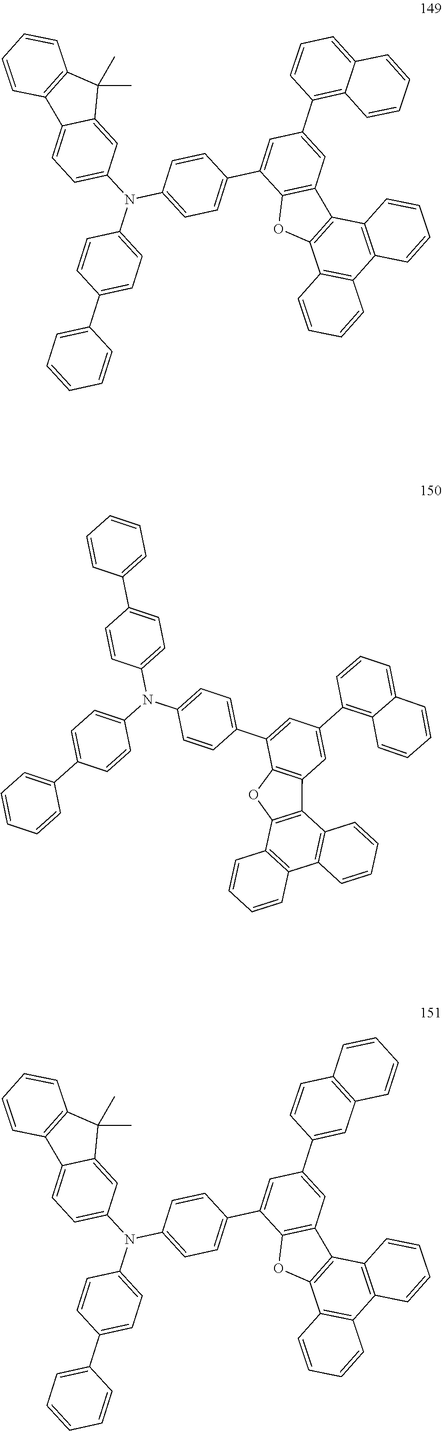 [SMILES: CC1(C)c2ccccc2-c2ccc(N(c3ccc(-c4ccccc4)cc3)c3ccc(-c4cc(-c5cccc6ccccc56)cc5c4oc4c6ccccc6c6ccccc6c54)cc3)cc21 ; c1ccc(-c2ccc(N(c3ccc(-c4ccccc4)cc3)c3ccc(-c4cc(-c5cccc6ccccc56)cc5c4oc4c6ccccc6c6ccccc6c54)cc3)cc2)cc1 ; CC1(C)c2ccccc2-c2ccc(N(c3ccc(-c4ccccc4)cc3)c3ccc(-c4cc(-c5ccc6ccccc6c5)cc5c4oc4c6ccccc6c6ccccc6c54)cc3)cc21]

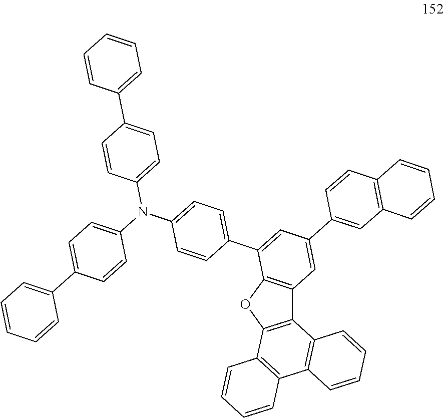 [SMILES: c1ccc(-c2ccc(N(c3ccc(-c4ccccc4)cc3)c3ccc(-c4cc(-c5ccc6ccccc6c5)cc5c4oc4c6ccccc6c6ccccc6c54)cc3)cc2)cc1]

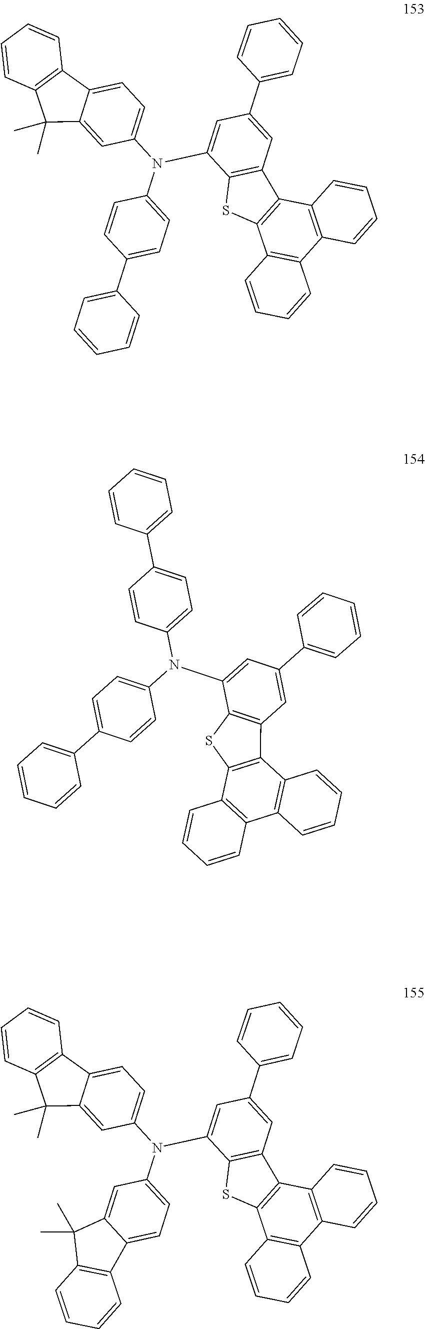 [SMILES: CC1(C)c2ccccc2-c2ccc(N(c3ccc(-c4ccccc4)cc3)c3cc(-c4ccccc4)cc4c3sc3c5ccccc5c5ccccc5c43)cc21 ; c1ccc(-c2ccc(N(c3ccc(-c4ccccc4)cc3)c3cc(-c4ccccc4)cc4c3sc3c5ccccc5c5ccccc5c43)cc2)cc1 ; CC1(C)c2ccccc2-c2ccc(N(c3ccc4c(c3)C(C)(C)c3ccccc3-4)c3cc(-c4ccccc4)cc4c3sc3c5ccccc5c5ccccc5c43)cc21]

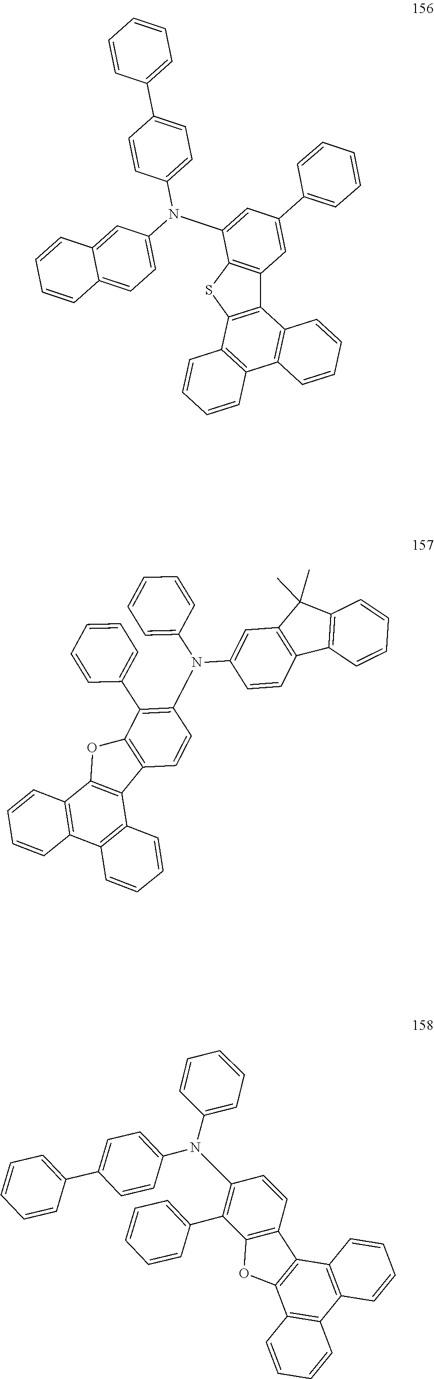 [SMILES: c1ccc(-c2ccc(N(c3ccc4ccccc4c3)c3cc(-c4ccccc4)cc4c3sc3c5ccccc5c5ccccc5c43)cc2)cc1 ; CC1(C)c2ccccc2-c2ccc(N(c3ccccc3)c3ccc4c(oc5c6ccccc6c6ccccc6c45)c3-c3ccccc3)cc21 ; c1ccc(-c2ccc(N(c3ccccc3)c3ccc4c(oc5c6ccccc6c6ccccc6c45)c3-c3ccccc3)cc2)cc1]

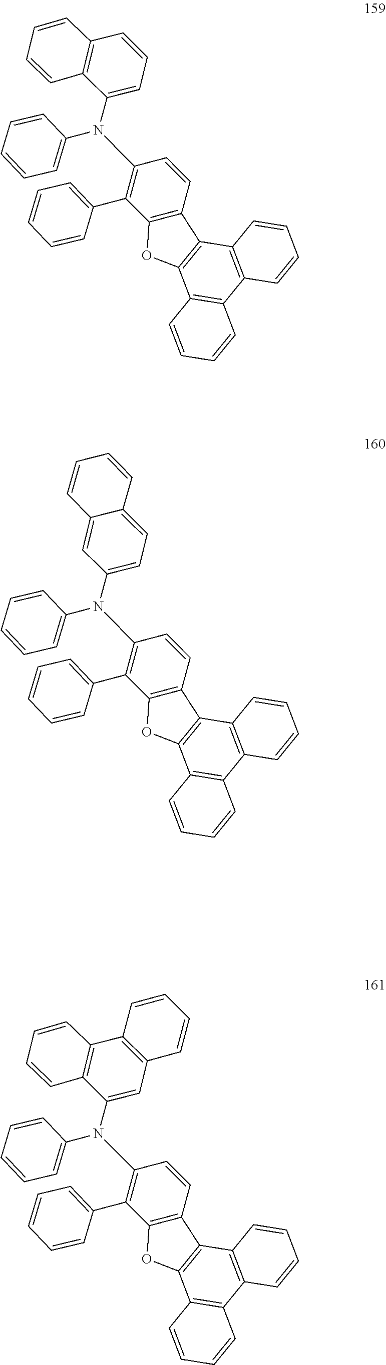 [SMILES: c1ccc(-c2c(N(c3ccccc3)c3cccc4ccccc34)ccc3c2oc2c4ccccc4c4ccccc4c32)cc1 ; c1ccc(-c2c(N(c3ccccc3)c3ccc4ccccc4c3)ccc3c2oc2c4ccccc4c4ccccc4c32)cc1 ; c1ccc(-c2c(N(c3ccccc3)c3cc4ccccc4c4ccccc34)ccc3c2oc2c4ccccc4c4ccccc4c32)cc1]

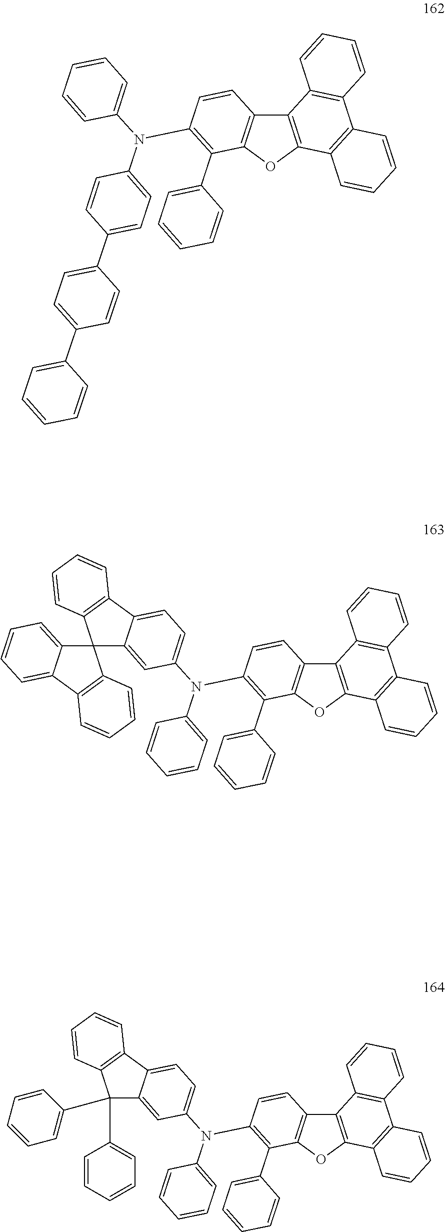 [SMILES: c1ccc(-c2ccc(-c3ccc(N(c4ccccc4)c4ccc5c(oc6c7ccccc7c7ccccc7c56)c4-c4ccccc4)cc3)cc2)cc1 ; c1ccc(-c2c(N(c3ccccc3)c3ccc4c(c3)C3(c5ccccc5-c5ccccc53)c3ccccc3-4)ccc3c2oc2c4ccccc4c4ccccc4c32)cc1 ; c1ccc(-c2c(N(c3ccccc3)c3ccc4c(c3)C(c3ccccc3)(c3ccccc3)c3ccccc3-4)ccc3c2oc2c4ccccc4c4ccccc4c32)cc1]

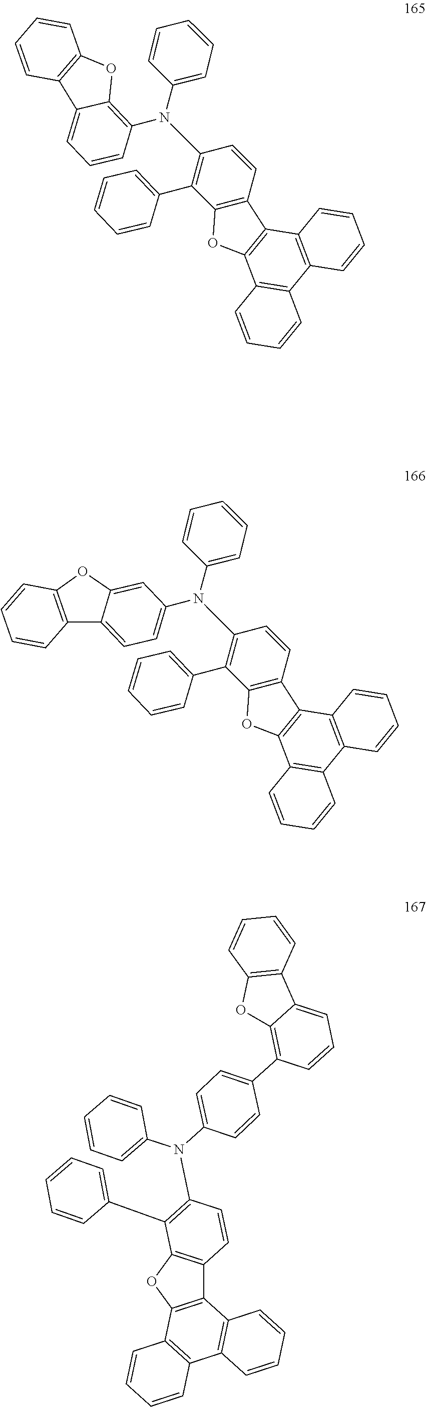 [SMILES: c1ccc(-c2c(N(c3ccccc3)c3cccc4c3oc3ccccc34)ccc3c2oc2c4ccccc4c4ccccc4c32)cc1 ; c1ccc(-c2c(N(c3ccccc3)c3ccc4c(c3)oc3ccccc34)ccc3c2oc2c4ccccc4c4ccccc4c32)cc1 ; c1ccc(-c2c(N(c3ccccc3)c3ccc(-c4cccc5c4oc4ccccc45)cc3)ccc3c2oc2c4ccccc4c4ccccc4c32)cc1]

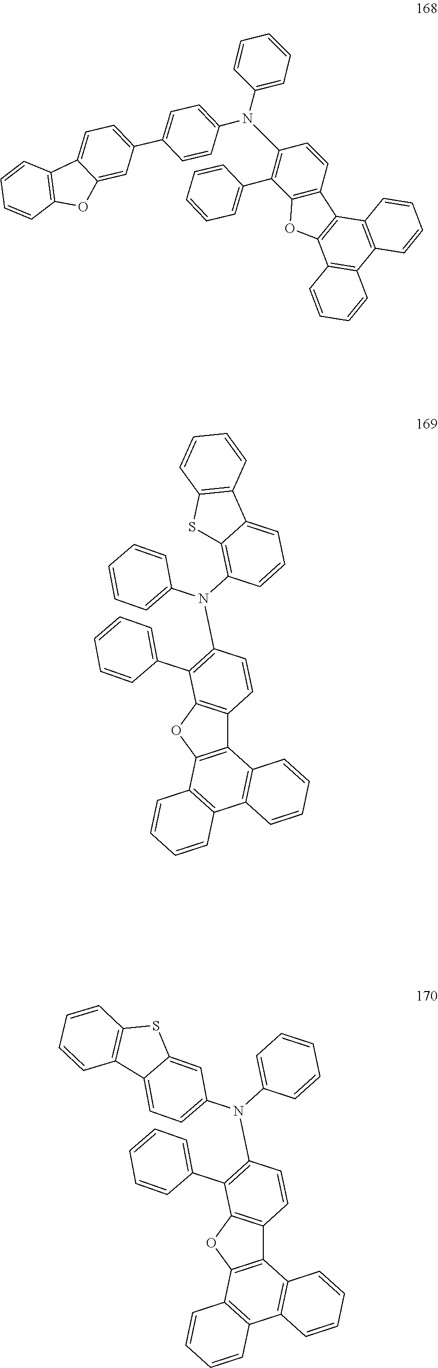 [SMILES: c1ccc(-c2c(N(c3ccccc3)c3ccc(-c4ccc5c(c4)oc4ccccc45)cc3)ccc3c2oc2c4ccccc4c4ccccc4c32)cc1 ; c1ccc(-c2c(N(c3ccccc3)c3cccc4c3sc3ccccc34)ccc3c2oc2c4ccccc4c4ccccc4c32)cc1 ; c1ccc(-c2c(N(c3ccccc3)c3ccc4c(c3)sc3ccccc34)ccc3c2oc2c4ccccc4c4ccccc4c32)cc1]

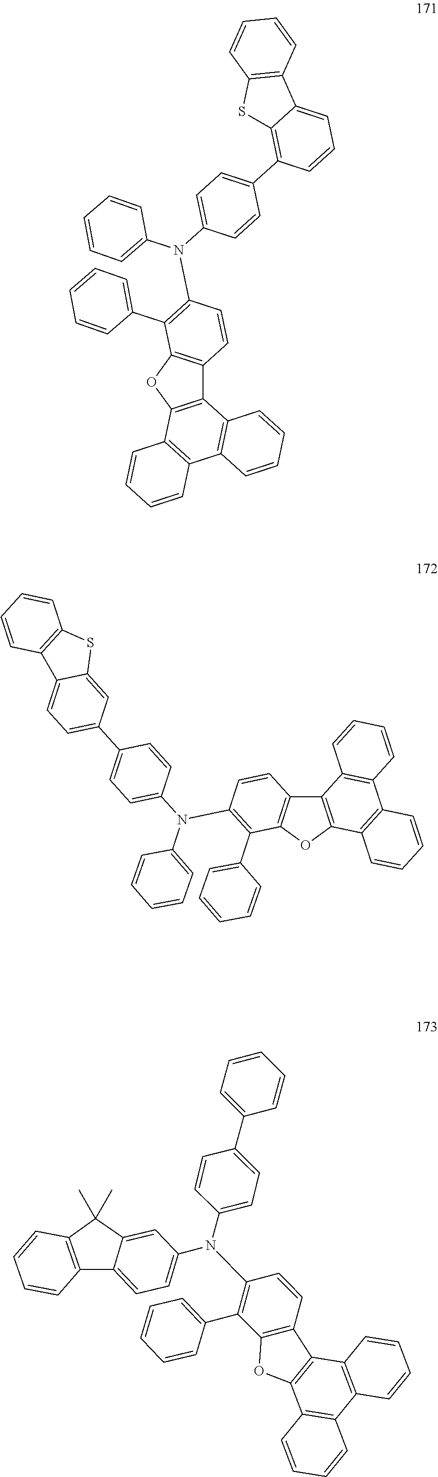 [SMILES: c1ccc(-c2c(N(c3ccccc3)c3ccc(-c4cccc5c4sc4ccccc45)cc3)ccc3c2oc2c4ccccc4c4ccccc4c32)cc1 ; c1ccc(-c2c(N(c3ccccc3)c3ccc(-c4ccc5c(c4)sc4ccccc45)cc3)ccc3c2oc2c4ccccc4c4ccccc4c32)cc1 ; CC1(C)c2ccccc2-c2ccc(N(c3ccc(-c4ccccc4)cc3)c3ccc4c(oc5c6ccccc6c6ccccc6c45)c3-c3ccccc3)cc21]

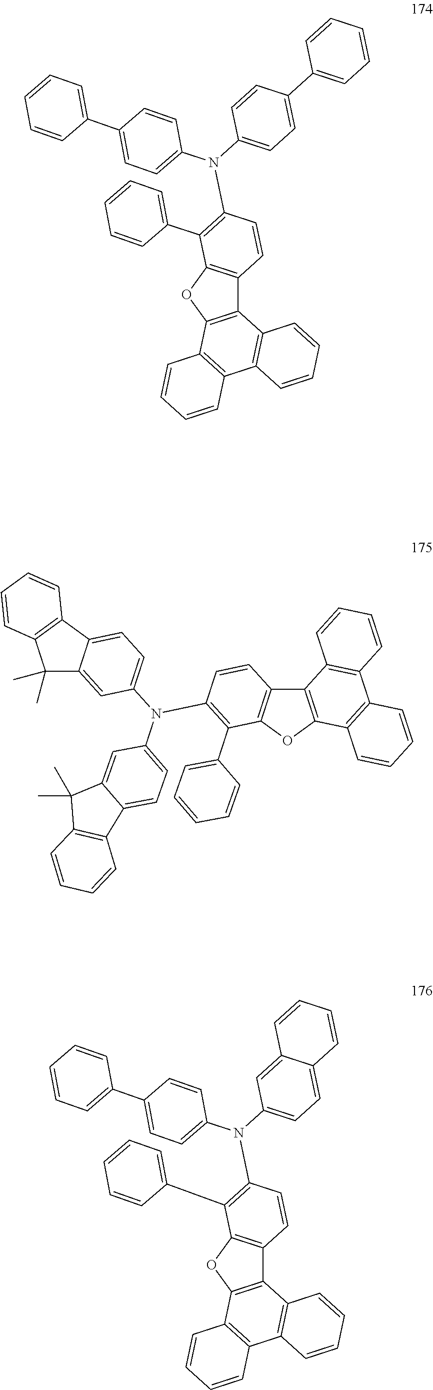 [SMILES: c1ccc(-c2ccc(N(c3ccc(-c4ccccc4)cc3)c3ccc4c(oc5c6ccccc6c6ccccc6c45)c3-c3ccccc3)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccccc3)cc21 ; c1ccc(-c2ccc(N(c3ccc4ccccc4c3)c3ccc4c(oc5c6ccccc6c6ccccc6c45)c3-c3ccccc3)cc2)cc1]

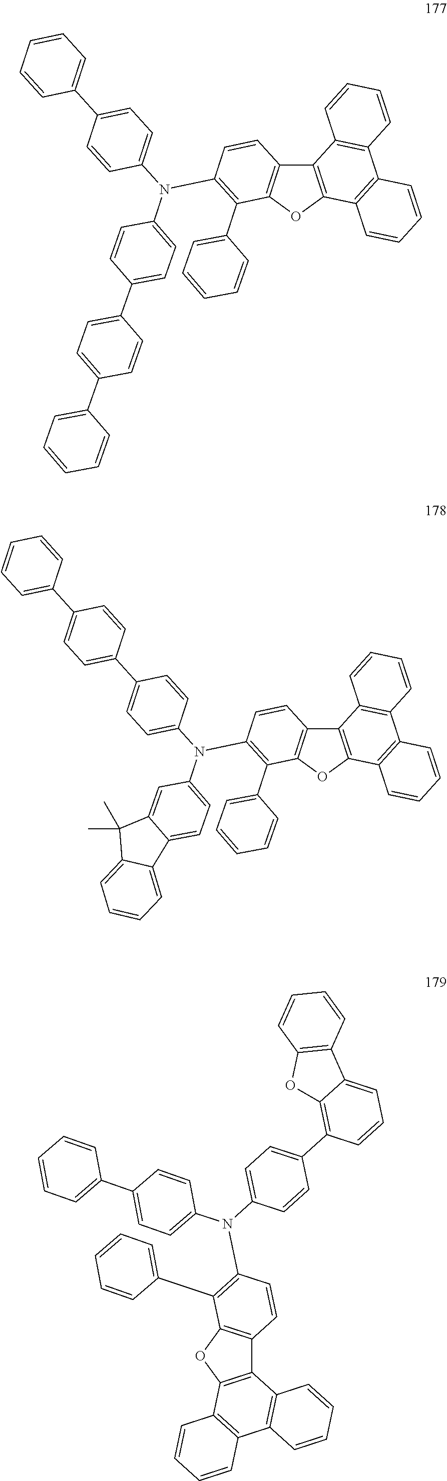 [SMILES: c1ccc(-c2ccc(-c3ccc(N(c4ccc(-c5ccccc5)cc4)c4ccc5c(oc6c7ccccc7c7ccccc7c56)c4-c4ccccc4)cc3)cc2)cc1 ; CC1(C)c2ccccc2-c2ccc(N(c3ccc(-c4ccc(-c5ccccc5)cc4)cc3)c3ccc4c(oc5c6ccccc6c6ccccc6c45)c3-c3ccccc3)cc21 ; c1ccc(-c2ccc(N(c3ccc(-c4cccc5c4oc4ccccc45)cc3)c3ccc4c(oc5c6ccccc6c6ccccc6c45)c3-c3ccccc3)cc2)cc1]

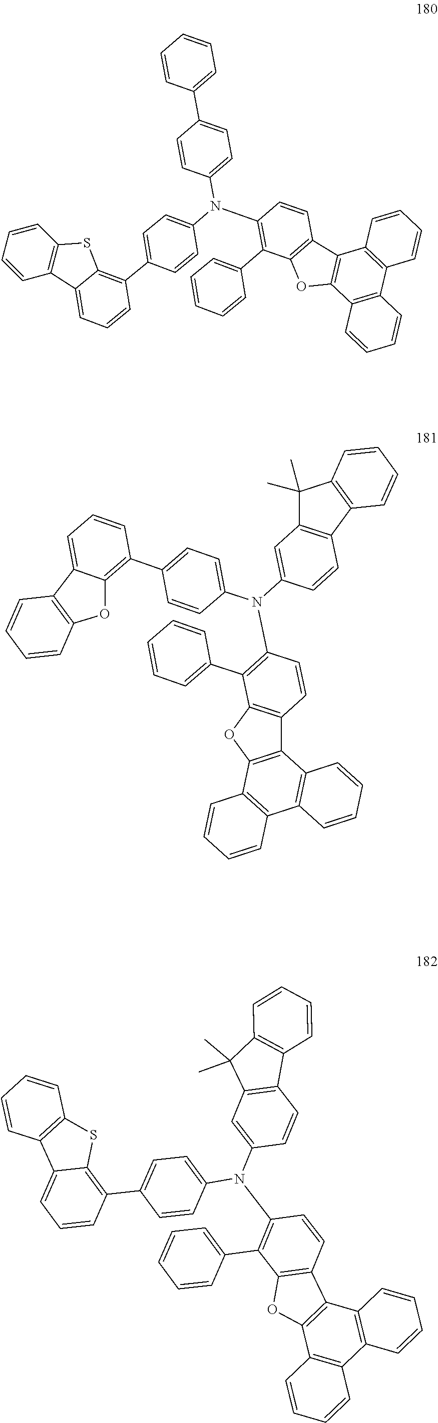 [SMILES: c1ccc(-c2ccc(N(c3ccc(-c4cccc5c4sc4ccccc45)cc3)c3ccc4c(oc5c6ccccc6c6ccccc6c45)c3-c3ccccc3)cc2)cc1 ; CC1(C)c2ccccc2-c2ccc(N(c3ccc(-c4cccc5c4oc4ccccc45)cc3)c3ccc4c(oc5c6ccccc6c6ccccc6c45)c3-c3ccccc3)cc21 ; CC1(C)c2ccccc2-c2ccc(N(c3ccc(-c4cccc5c4sc4ccccc45)cc3)c3ccc4c(oc5c6ccccc6c6ccccc6c45)c3-c3ccccc3)cc21]

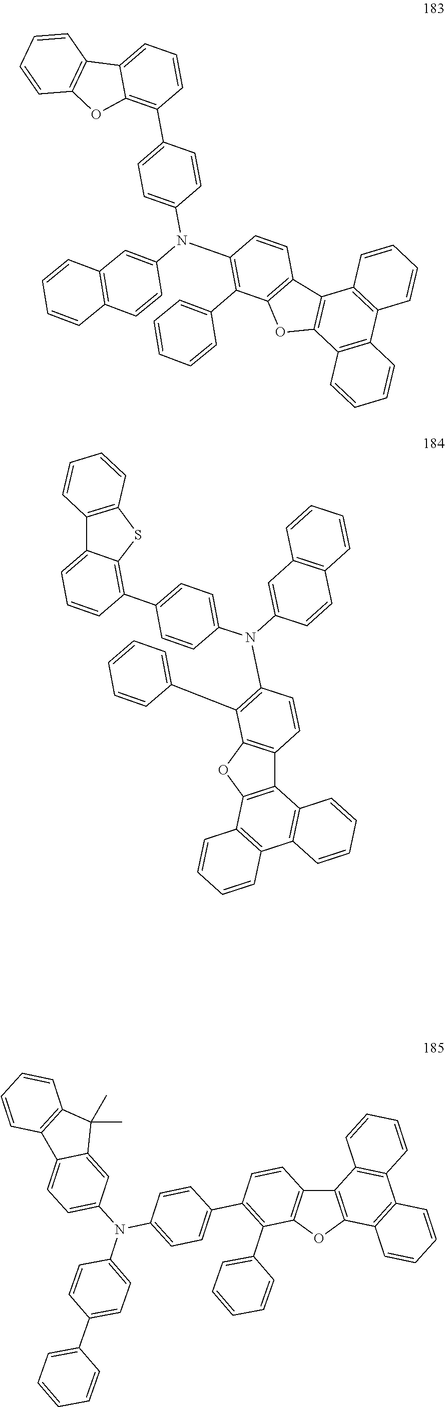 [SMILES: c1ccc(-c2c(N(c3ccc(-c4cccc5c4oc4ccccc45)cc3)c3ccc4ccccc4c3)ccc3c2oc2c4ccccc4c4ccccc4c32)cc1 ; c1ccc(-c2c(N(c3ccc(-c4cccc5c4sc4ccccc45)cc3)c3ccc4ccccc4c3)ccc3c2oc2c4ccccc4c4ccccc4c32)cc1 ; CC1(C)c2ccccc2-c2ccc(N(c3ccc(-c4ccccc4)cc3)c3ccc(-c4ccc5c(oc6c7ccccc7c7ccccc7c56)c4-c4ccccc4)cc3)cc21]

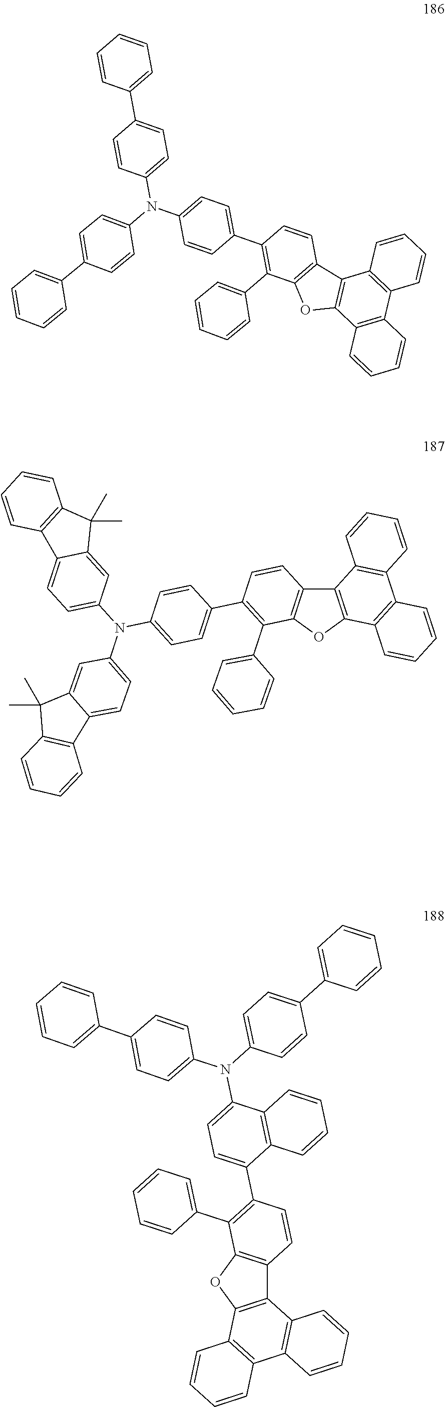 [SMILES: c1ccc(-c2ccc(N(c3ccc(-c4ccccc4)cc3)c3ccc(-c4ccc5c(oc6c7ccccc7c7ccccc7c56)c4-c4ccccc4)cc3)cc2)cc1 ; CC1(C)c2ccccc2-c2ccc(N(c3ccc(-c4ccc5c(oc6c7ccccc7c7ccccc7c56)c4-c4ccccc4)cc3)c3ccc4c(c3)C(C)(C)c3ccccc3-4)cc21 ; c1ccc(-c2ccc(N(c3ccc(-c4ccccc4)cc3)c3ccc(-c4ccc5c(oc6c7ccccc7c7ccccc7c56)c4-c4ccccc4)c4ccccc34)cc2)cc1]

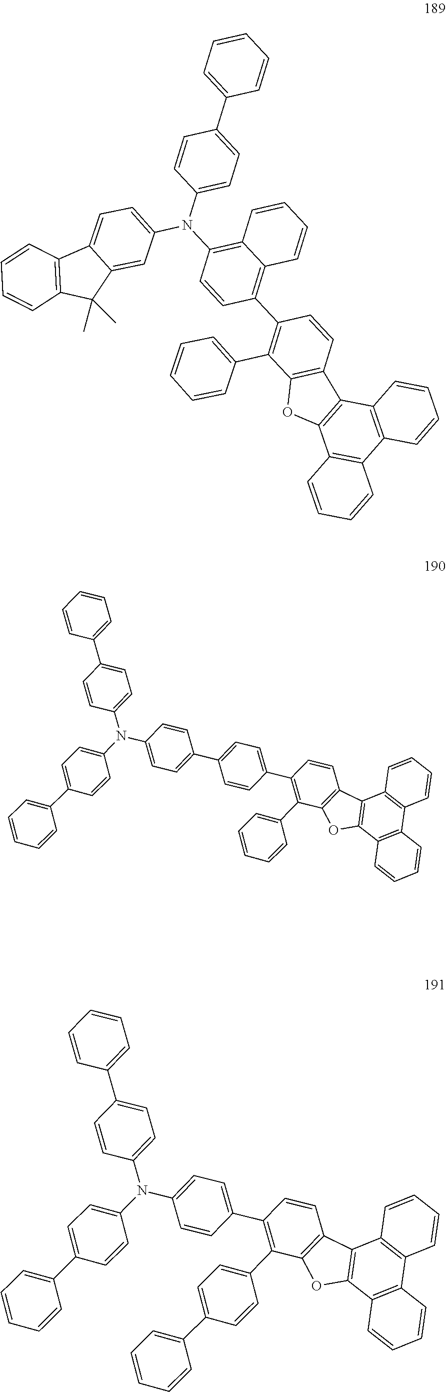 [SMILES: CC1(C)c2ccccc2-c2ccc(N(c3ccc(-c4ccccc4)cc3)c3ccc(-c4ccc5c(oc6c7ccccc7c7ccccc7c56)c4-c4ccccc4)c4ccccc34)cc21 ; c1ccc(-c2ccc(N(c3ccc(-c4ccccc4)cc3)c3ccc(-c4ccc(-c5ccc6c(oc7c8ccccc8c8ccccc8c67)c5-c5ccccc5)cc4)cc3)cc2)cc1 ; c1ccc(-c2ccc(-c3c(-c4ccc(N(c5ccc(-c6ccccc6)cc5)c5ccc(-c6ccccc6)cc5)cc4)ccc4c3oc3c5ccccc5c5ccccc5c43)cc2)cc1]

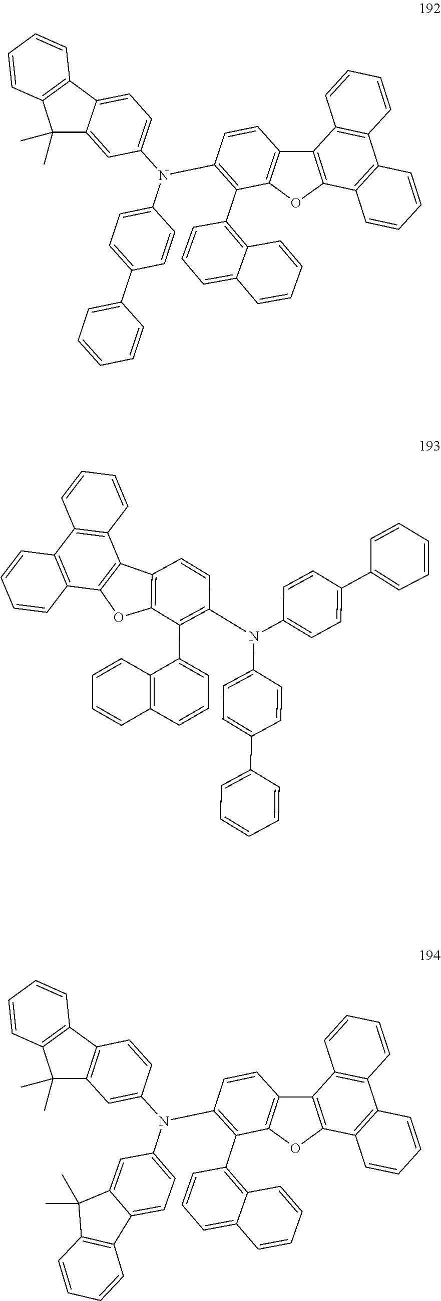 [SMILES: CC1(C)c2ccccc2-c2ccc(N(c3ccc(-c4ccccc4)cc3)c3ccc4c(oc5c6ccccc6c6ccccc6c45)c3-c3cccc4ccccc34)cc21 ; c1ccc(-c2ccc(N(c3ccc(-c4ccccc4)cc3)c3ccc4c(oc5c6ccccc6c6ccccc6c45)c3-c3cccc4ccccc34)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3cccc4ccccc34)cc21]

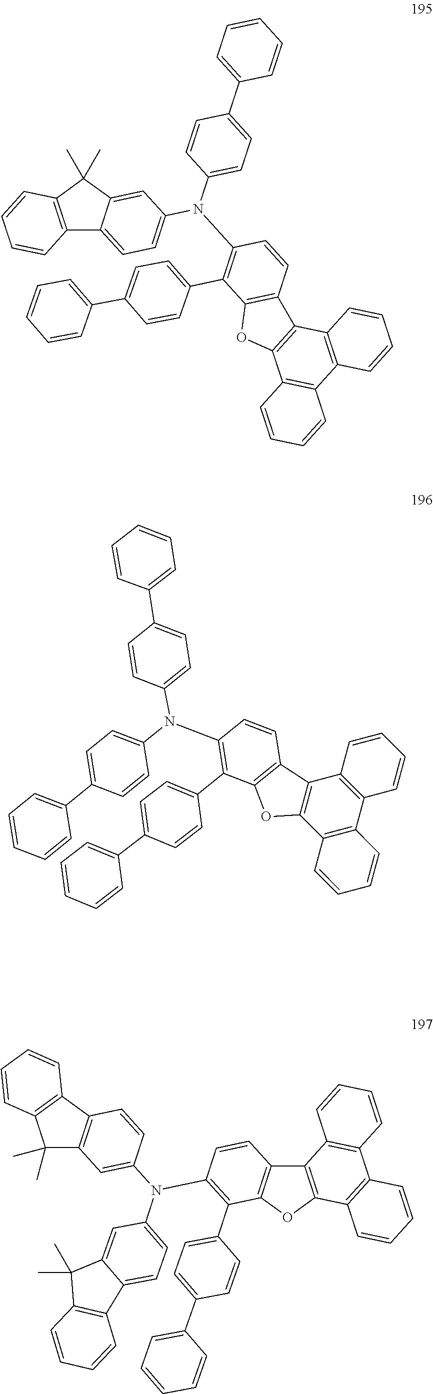 [SMILES: CC1(C)c2ccccc2-c2ccc(N(c3ccc(-c4ccccc4)cc3)c3ccc4c(oc5c6ccccc6c6ccccc6c45)c3-c3ccc(-c4ccccc4)cc3)cc21 ; c1ccc(-c2ccc(-c3c(N(c4ccc(-c5ccccc5)cc4)c4ccc(-c5ccccc5)cc4)ccc4c3oc3c5ccccc5c5ccccc5c43)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccc(-c4ccccc4)cc3)cc21]

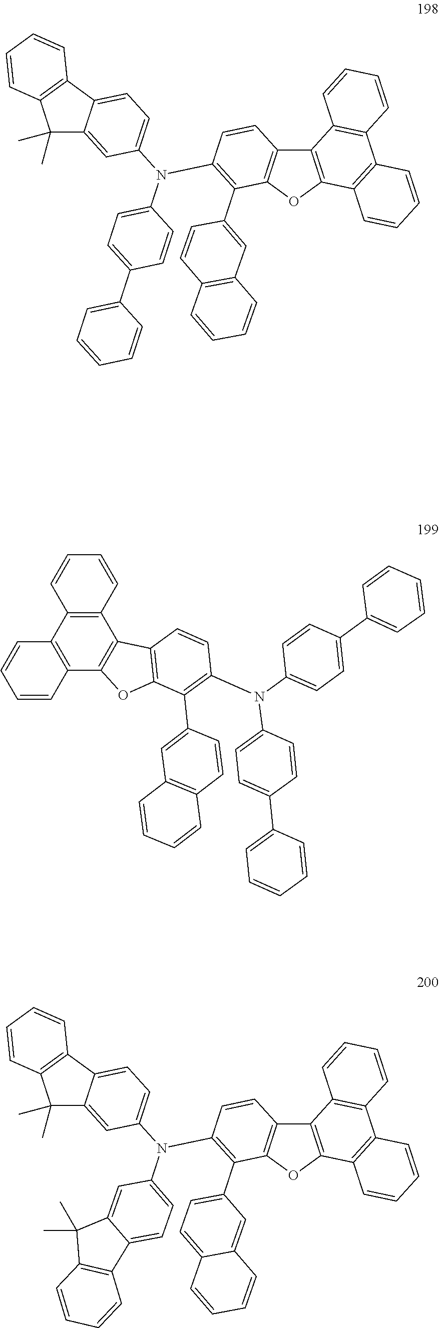 [SMILES: CC1(C)c2ccccc2-c2ccc(N(c3ccc(-c4ccccc4)cc3)c3ccc4c(oc5c6ccccc6c6ccccc6c45)c3-c3ccc4ccccc4c3)cc21 ; c1ccc(-c2ccc(N(c3ccc(-c4ccccc4)cc3)c3ccc4c(oc5c6ccccc6c6ccccc6c45)c3-c3ccc4ccccc4c3)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccc4ccccc4c3)cc21]

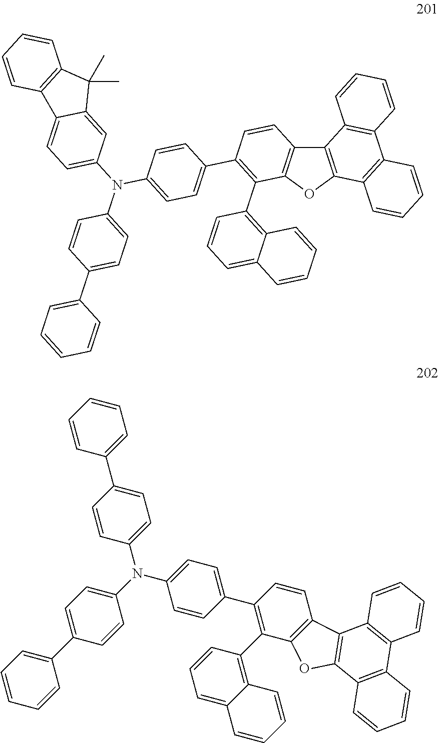 [SMILES: CC1(C)c2ccccc2-c2ccc(N(c3ccc(-c4ccccc4)cc3)c3ccc(-c4ccc5c(oc6c7ccccc7c7ccccc7c56)c4-c4cccc5ccccc45)cc3)cc21 ; c1ccc(-c2ccc(N(c3ccc(-c4ccccc4)cc3)c3ccc(-c4ccc5c(oc6c7ccccc7c7ccccc7c56)c4-c4cccc5ccccc45)cc3)cc2)cc1]

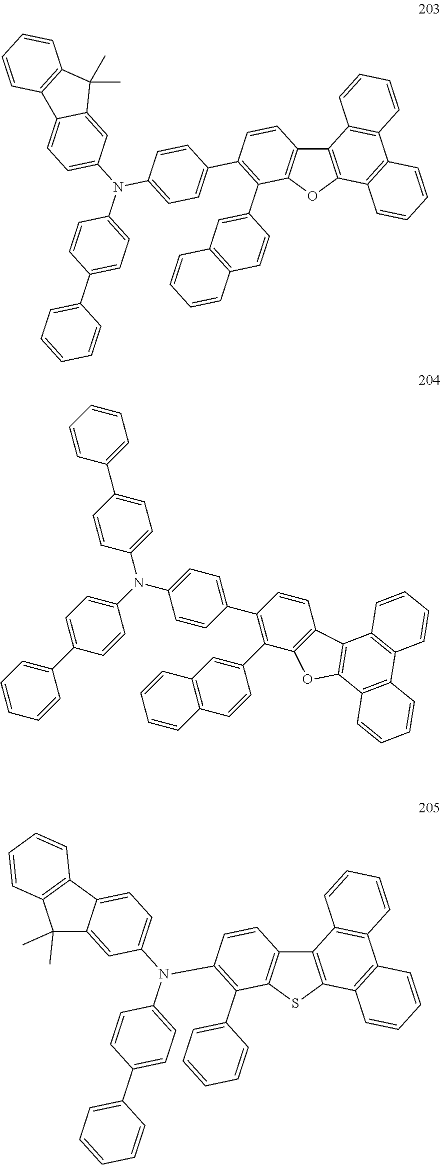 [SMILES: CC1(C)c2ccccc2-c2ccc(N(c3ccc(-c4ccccc4)cc3)c3ccc(-c4ccc5c(oc6c7ccccc7c7ccccc7c56)c4-c4ccc5ccccc5c4)cc3)cc21 ; c1ccc(-c2ccc(N(c3ccc(-c4ccccc4)cc3)c3ccc(-c4ccc5c(oc6c7ccccc7c7ccccc7c56)c4-c4ccc5ccccc5c4)cc3)cc2)cc1 ; CC1(C)c2ccccc2-c2ccc(N(c3ccc(-c4ccccc4)cc3)c3ccc4c(sc5c6ccccc6c6ccccc6c45)c3-c3ccccc3)cc21]

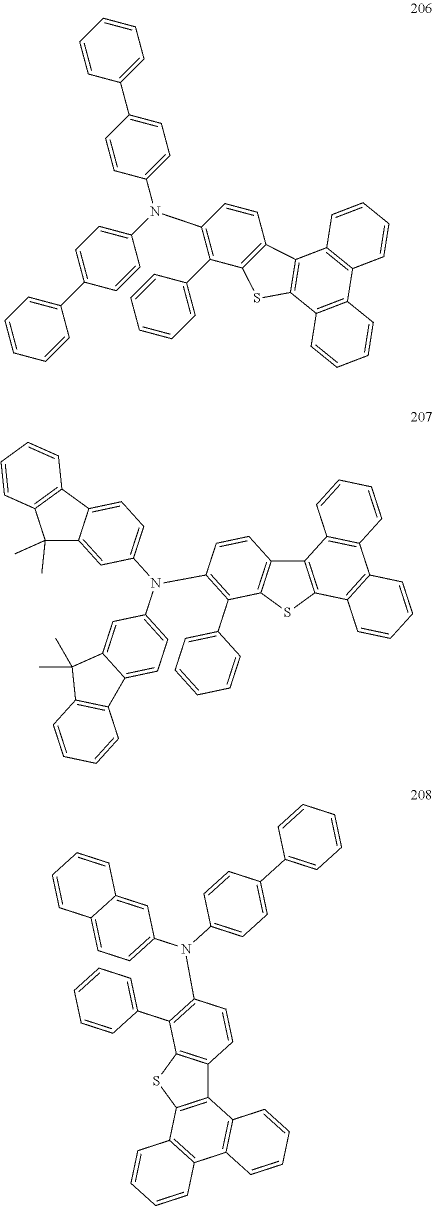 [SMILES: c1ccc(-c2ccc(N(c3ccc(-c4ccccc4)cc3)c3ccc4c(sc5c6ccccc6c6ccccc6c45)c3-c3ccccc3)cc2)cc1 ; CC1(C)c2ccccc2-c2ccc(N(c3ccc4c(c3)C(C)(C)c3ccccc3-4)c3ccc4c(sc5c6ccccc6c6ccccc6c45)c3-c3ccccc3)cc21 ; c1ccc(-c2ccc(N(c3ccc4ccccc4c3)c3ccc4c(sc5c6ccccc6c6ccccc6c45)c3-c3ccccc3)cc2)cc1]

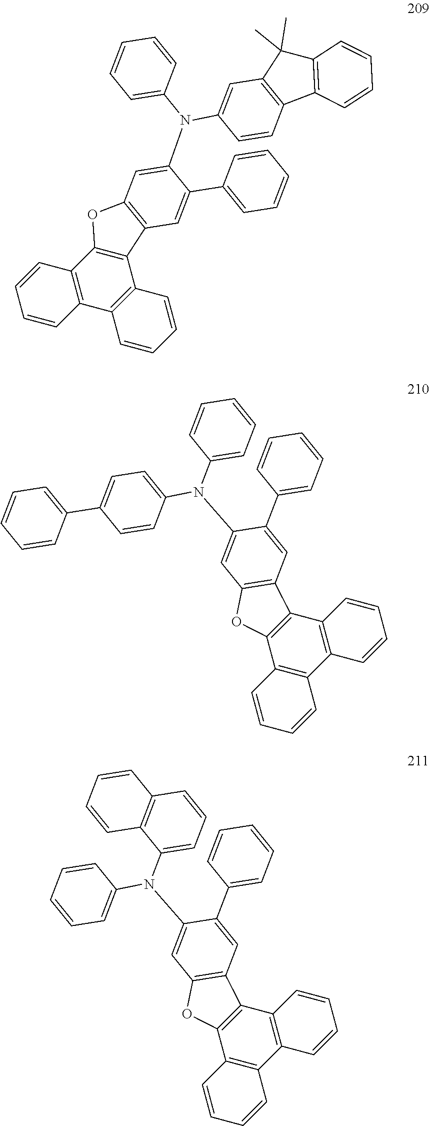 [SMILES: CC1(C)c2ccccc2-c2ccc(N(c3ccccc3)c3cc4oc5c6ccccc6c6ccccc6c5c4cc3-c3ccccc3)cc21 ; c1ccc(-c2ccc(N(c3ccccc3)c3cc4oc5c6ccccc6c6ccccc6c5c4cc3-c3ccccc3)cc2)cc1 ; c1ccc(-c2cc3c(cc2N(c2ccccc2)c2cccc4ccccc24)oc2c4ccccc4c4ccccc4c32)cc1]

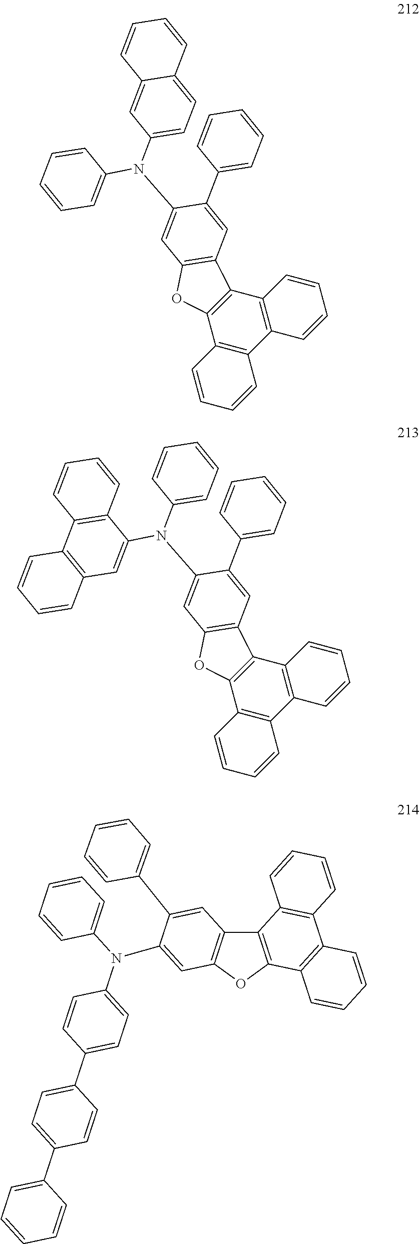

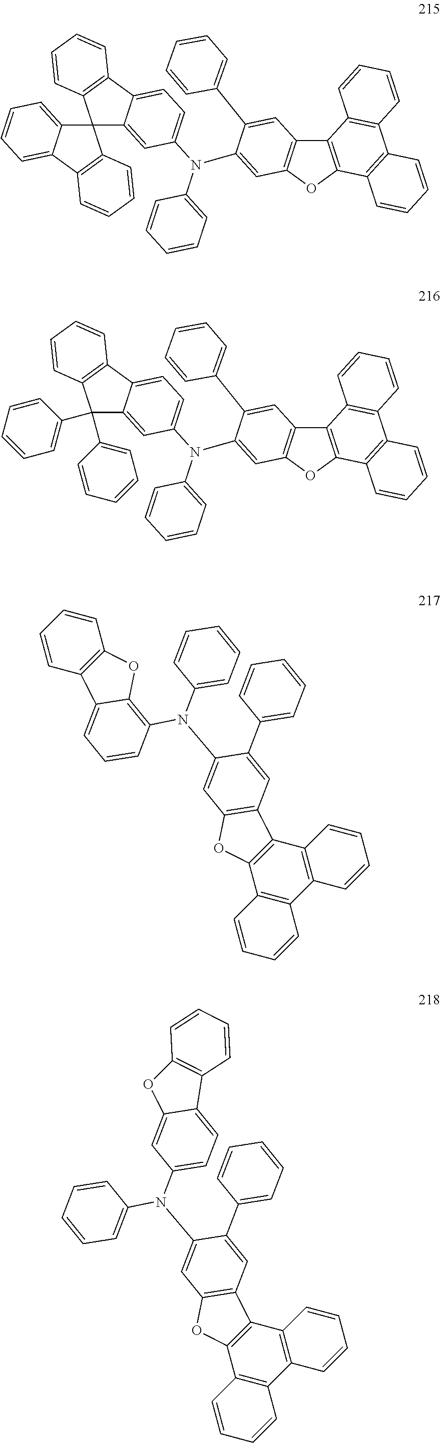 [SMILES: c1ccc(-c2cc3c(cc2N(c2ccccc2)c2ccc4c(c2)C2(c5ccccc5-c5ccccc52)c2ccccc2-4)oc2c4ccccc4c4ccccc4c32)cc1 ; c1ccc(-c2cc3c(cc2N(c2ccccc2)c2ccc4c(c2)C(c2ccccc2)(c2ccccc2)c2ccccc2-4)oc2c4ccccc4c4ccccc4c32)cc1 ; c1ccc(-c2cc3c(cc2N(c2ccccc2)c2cccc4c2oc2ccccc24)oc2c4ccccc4c4ccccc4c32)cc1 ; c1ccc(-c2cc3c(cc2N(c2ccccc2)c2ccc4c(c2)oc2ccccc24)oc2c4ccccc4c4ccccc4c32)cc1]

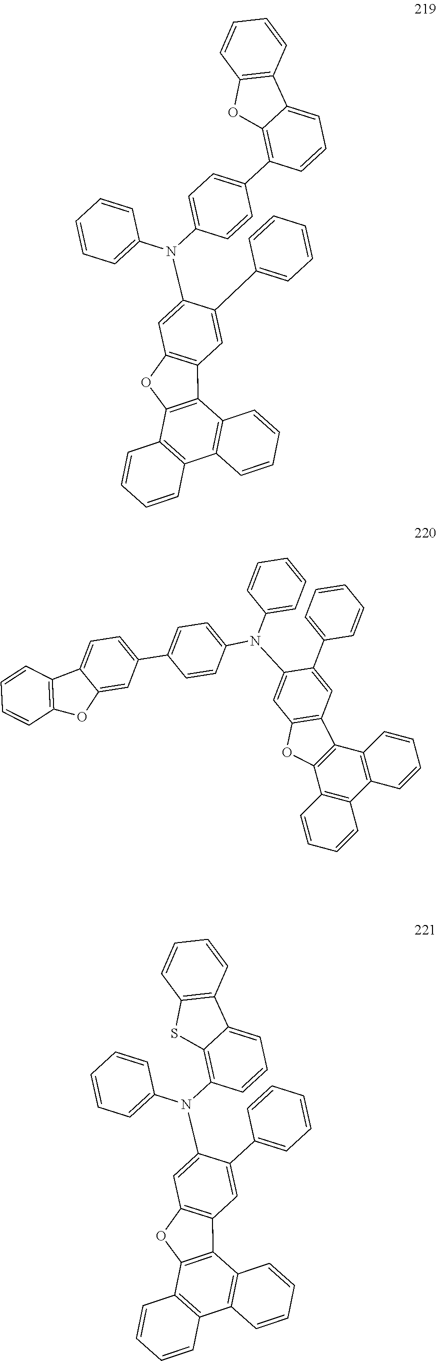 [SMILES: c1ccc(-c2cc3c(cc2N(c2ccccc2)c2ccc(-c4cccc5c4oc4ccccc45)cc2)oc2c4ccccc4c4ccccc4c32)cc1 ; c1ccc(-c2cc3c(cc2N(c2ccccc2)c2ccc(-c4ccc5c(c4)oc4ccccc45)cc2)oc2c4ccccc4c4ccccc4c32)cc1 ; c1ccc(-c2cc3c(cc2N(c2ccccc2)c2cccc4c2sc2ccccc24)oc2c4ccccc4c4ccccc4c32)cc1]

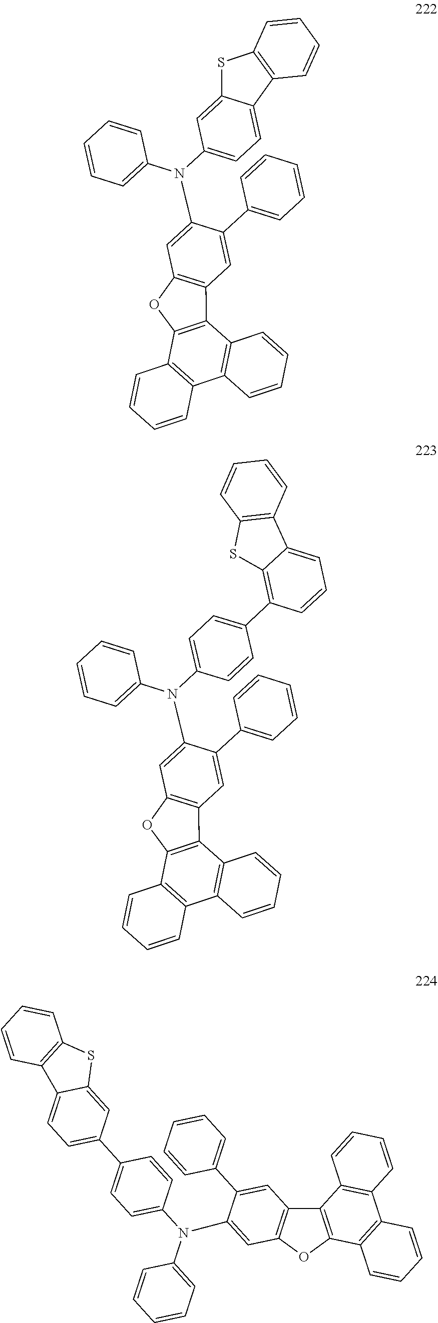 [SMILES: c1ccc(-c2cc3c(cc2N(c2ccccc2)c2ccc4c(c2)sc2ccccc24)oc2c4ccccc4c4ccccc4c32)cc1 ; c1ccc(-c2cc3c(cc2N(c2ccccc2)c2ccc(-c4cccc5c4sc4ccccc45)cc2)oc2c4ccccc4c4ccccc4c32)cc1 ; c1ccc(-c2cc3c(cc2N(c2ccccc2)c2ccc(-c4ccc5c(c4)sc4ccccc45)cc2)oc2c4ccccc4c4ccccc4c32)cc1]

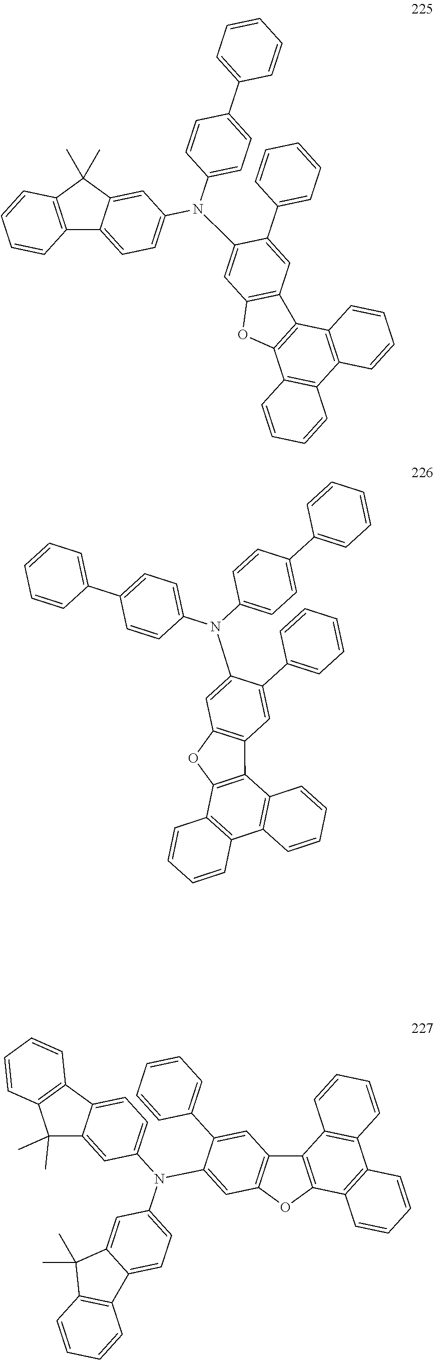 [SMILES: CC1(C)c2ccccc2-c2ccc(N(c3ccc(-c4ccccc4)cc3)c3cc4oc5c6ccccc6c6ccccc6c5c4cc3-c3ccccc3)cc21 ; c1ccc(-c2ccc(N(c3ccc(-c4ccccc4)cc3)c3cc4oc5c6ccccc6c6ccccc6c5c4cc3-c3ccccc3)cc2)cc1 ; CC1(C)c2ccccc2-c2ccc(N(c3ccc4c(c3)C(C)(C)c3ccccc3-4)c3cc4oc5c6ccccc6c6ccccc6c5c4cc3-c3ccccc3)cc21]

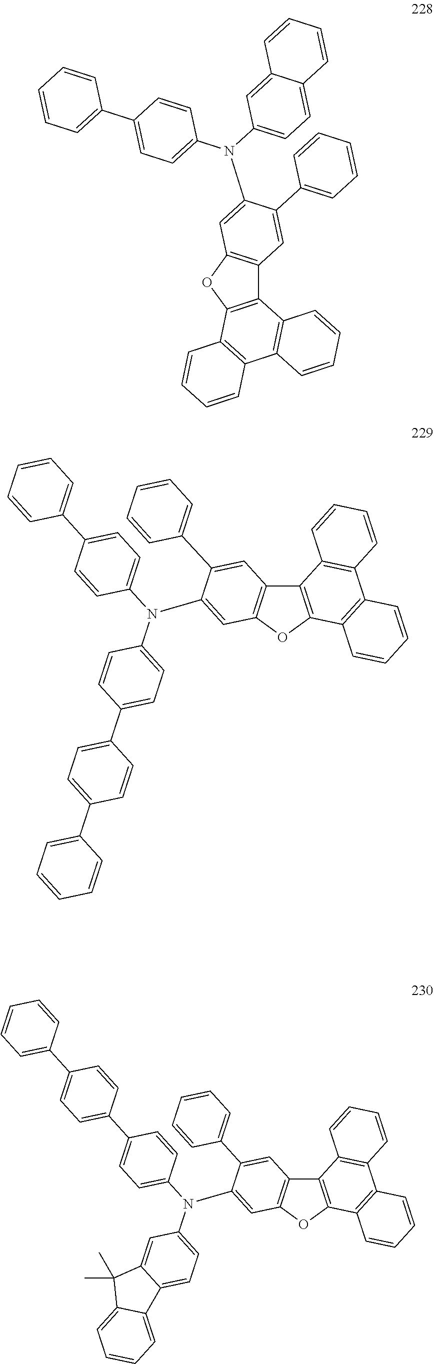 [SMILES: c1ccc(-c2ccc(N(c3ccc4ccccc4c3)c3cc4oc5c6ccccc6c6ccccc6c5c4cc3-c3ccccc3)cc2)cc1 ; c1ccc(-c2ccc(-c3ccc(N(c4ccc(-c5ccccc5)cc4)c4cc5oc6c7ccccc7c7ccccc7c6c5cc4-c4ccccc4)cc3)cc2)cc1 ; CC1(C)c2ccccc2-c2ccc(N(c3ccc(-c4ccc(-c5ccccc5)cc4)cc3)c3cc4oc5c6ccccc6c6ccccc6c5c4cc3-c3ccccc3)cc21]

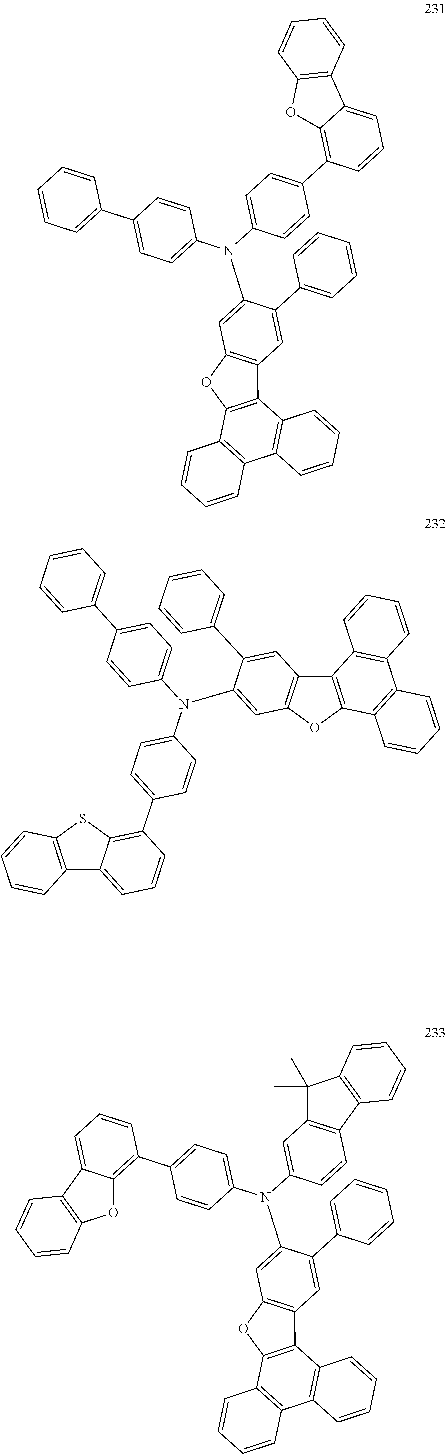 [SMILES: c1ccc(-c2ccc(N(c3ccc(-c4cccc5c4oc4ccccc45)cc3)c3cc4oc5c6ccccc6c6ccccc6c5c4cc3-c3ccccc3)cc2)cc1 ; c1ccc(-c2ccc(N(c3ccc(-c4cccc5c4sc4ccccc45)cc3)c3cc4oc5c6ccccc6c6ccccc6c5c4cc3-c3ccccc3)cc2)cc1 ; CC1(C)c2ccccc2-c2ccc(N(c3ccc(-c4cccc5c4oc4ccccc45)cc3)c3cc4oc5c6ccccc6c6ccccc6c5c4cc3-c3ccccc3)cc21]

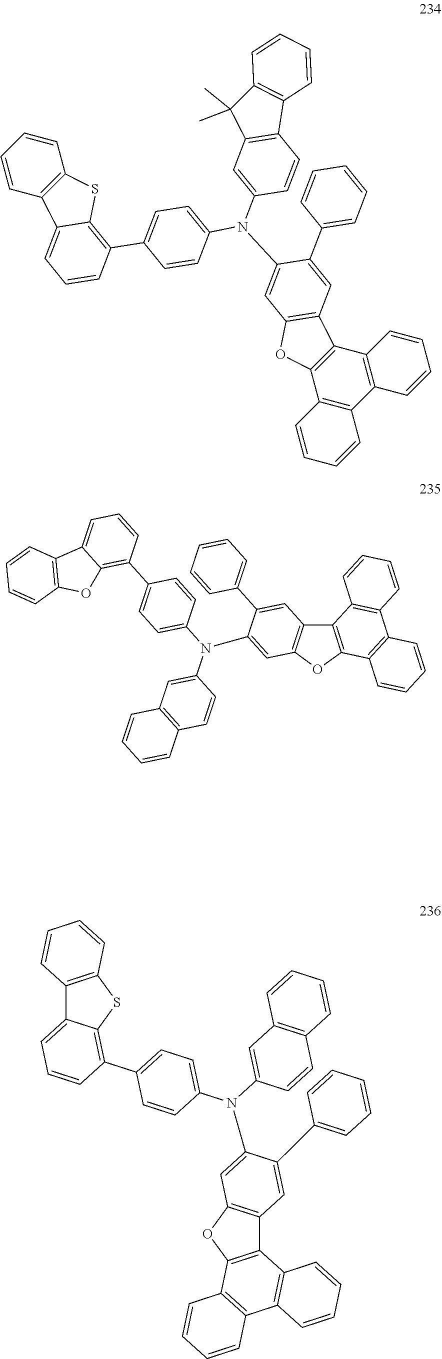 [SMILES: CC1(C)c2ccccc2-c2ccc(N(c3ccc(-c4cccc5c4sc4ccccc45)cc3)c3cc4oc5c6ccccc6c6ccccc6c5c4cc3-c3ccccc3)cc21 ; c1ccc(-c2cc3c(cc2N(c2ccc(-c4cccc5c4oc4ccccc45)cc2)c2ccc4ccccc4c2)oc2c4ccccc4c4ccccc4c32)cc1 ; c1ccc(-c2cc3c(cc2N(c2ccc(-c4cccc5c4sc4ccccc45)cc2)c2ccc4ccccc4c2)oc2c4ccccc4c4ccccc4c32)cc1]

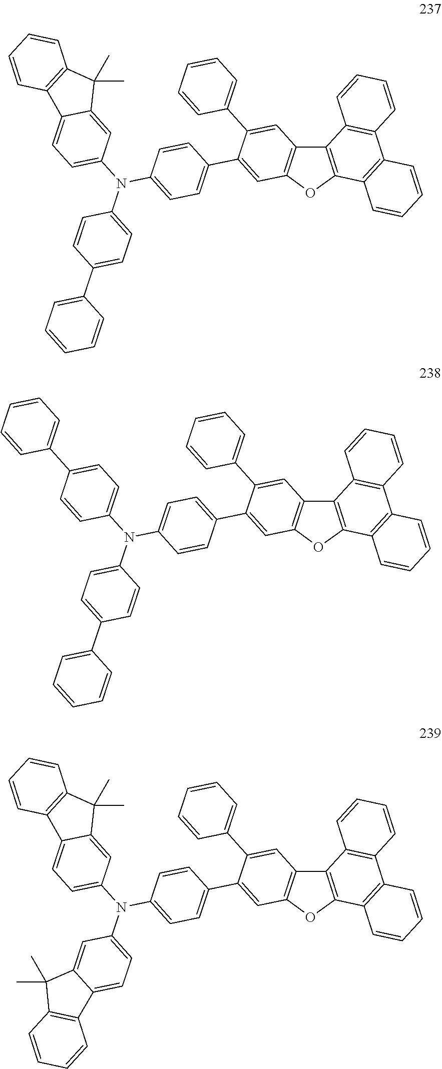 [SMILES: CC1(C)c2ccccc2-c2ccc(N(c3ccc(-c4ccccc4)cc3)c3ccc(-c4cc5oc6c7ccccc7c7ccccc7c6c5cc4-c4ccccc4)cc3)cc21 ; c1ccc(-c2ccc(N(c3ccc(-c4ccccc4)cc3)c3ccc(-c4cc5oc6c7ccccc7c7ccccc7c6c5cc4-c4ccccc4)cc3)cc2)cc1 ; CC1(C)c2ccccc2-c2ccc(N(c3ccc(-c4cc5oc6c7ccccc7c7ccccc7c6c5cc4-c4ccccc4)cc3)c3ccc4c(c3)C(C)(C)c3ccccc3-4)cc21]

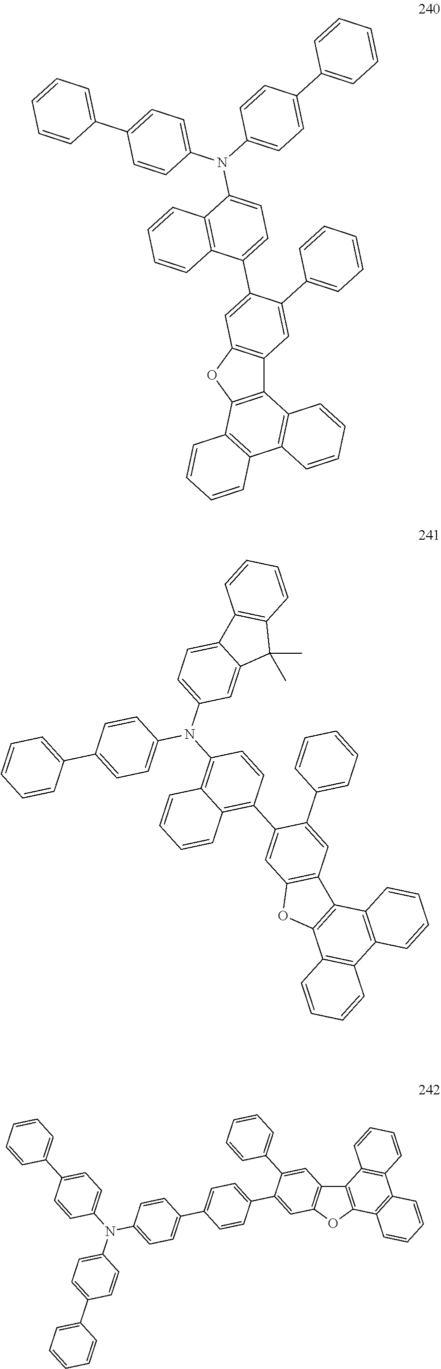 [SMILES: c1ccc(-c2ccc(N(c3ccc(-c4ccccc4)cc3)c3ccc(-c4cc5oc6c7ccccc7c7ccccc7c6c5cc4-c4ccccc4)c4ccccc34)cc2)cc1 ; CC1(C)c2ccccc2-c2ccc(N(c3ccc(-c4ccccc4)cc3)c3ccc(-c4cc5oc6c7ccccc7c7ccccc7c6c5cc4-c4ccccc4)c4ccccc34)cc21 ; c1ccc(-c2ccc(N(c3ccc(-c4ccccc4)cc3)c3ccc(-c4ccc(-c5cc6oc7c8ccccc8c8ccccc8c7c6cc5-c5ccccc5)cc4)cc3)cc2)cc1]

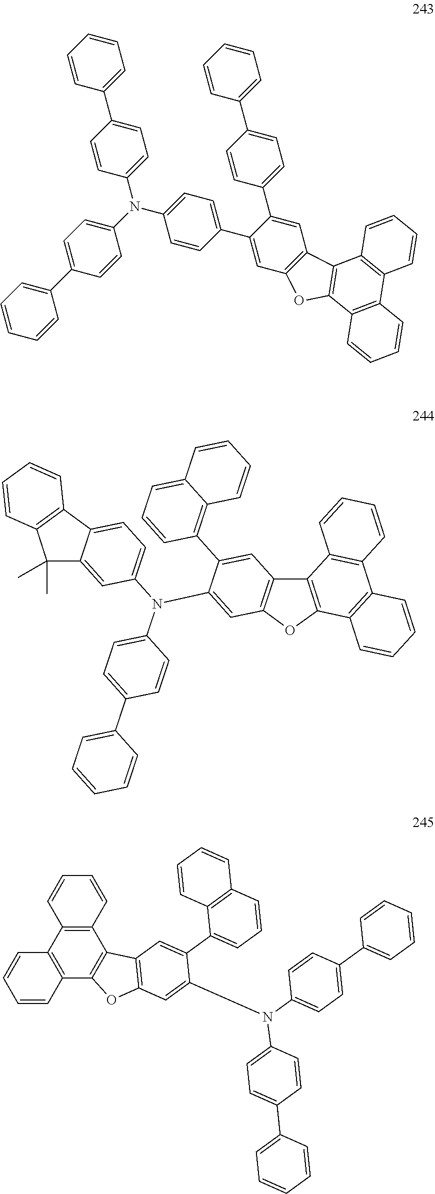 [SMILES: c1ccc(-c2ccc(-c3cc4c(cc3-c3ccc(N(c5ccc(-c6ccccc6)cc5)c5ccc(-c6ccccc6)cc5)cc3)oc3c5ccccc5c5ccccc5c43)cc2)cc1 ; CC1(C)c2ccccc2-c2ccc(N(c3ccc(-c4ccccc4)cc3)c3cc4oc5c6ccccc6c6ccccc6c5c4cc3-c3cccc4ccccc34)cc21 ; c1ccc(-c2ccc(N(c3ccc(-c4ccccc4)cc3)c3cc4oc5c6ccccc6c6ccccc6c5c4cc3-c3cccc4ccccc34)cc2)cc1]

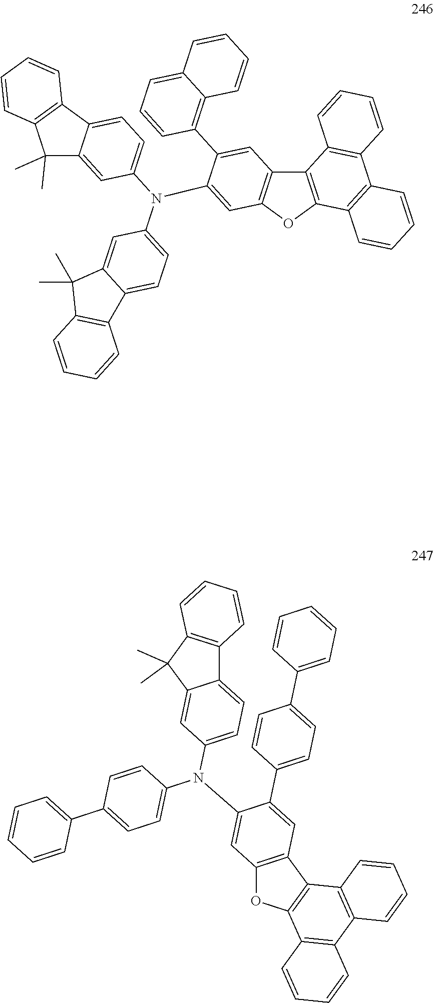 [SMILES: CC1(C)c2ccccc2-c2ccc(N(c3ccc4c(c3)C(C)(C)c3ccccc3-4)c3cc4oc5c6ccccc6c6ccccc6c5c4cc3-c3cccc4ccccc34)cc21 ; CC1(C)c2ccccc2-c2ccc(N(c3ccc(-c4ccccc4)cc3)c3cc4oc5c6ccccc6c6ccccc6c5c4cc3-c3ccc(-c4ccccc4)cc3)cc21]

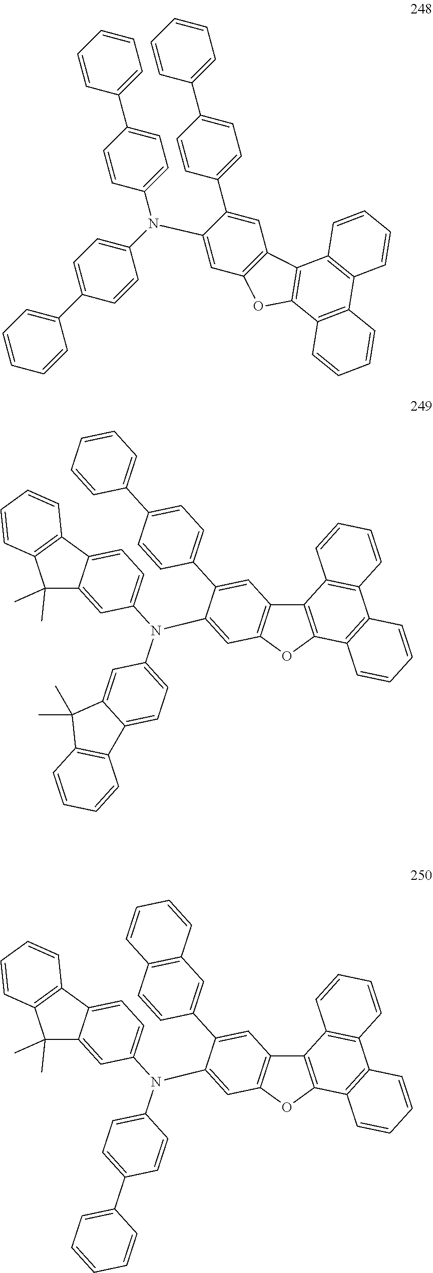 [SMILES: c1ccc(-c2ccc(-c3cc4c(cc3N(c3ccc(-c5ccccc5)cc3)c3ccc(-c5ccccc5)cc3)oc3c5ccccc5c5ccccc5c43)cc2)cc1 ; CC1(C)c2ccccc2-c2ccc(N(c3ccc4c(c3)C(C)(C)c3ccccc3-4)c3cc4oc5c6ccccc6c6ccccc6c5c4cc3-c3ccc(-c4ccccc4)cc3)cc21 ; CC1(C)c2ccccc2-c2ccc(N(c3ccc(-c4ccccc4)cc3)c3cc4oc5c6ccccc6c6ccccc6c5c4cc3-c3ccc4ccccc4c3)cc21]

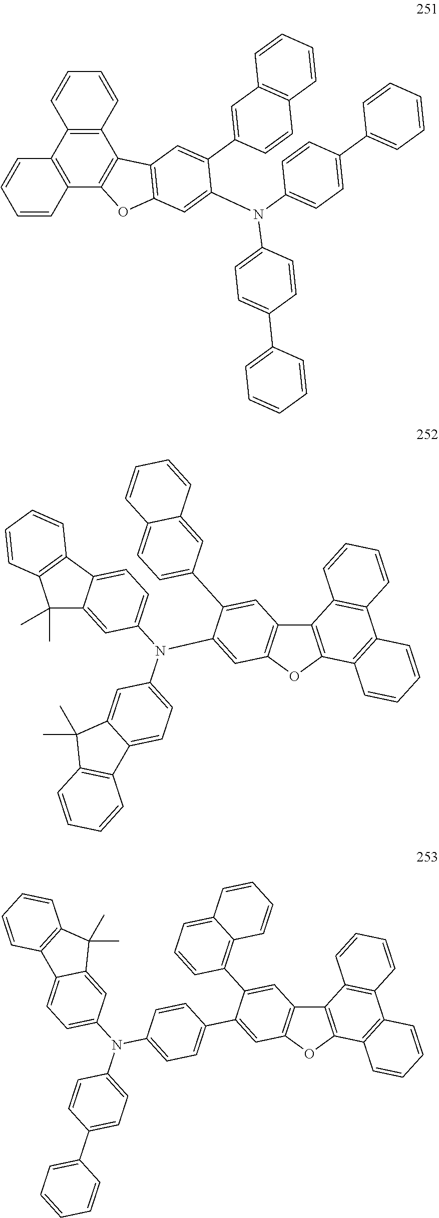 [SMILES: c1ccc(-c2ccc(N(c3ccc(-c4ccccc4)cc3)c3cc4oc5c6ccccc6c6ccccc6c5c4cc3-c3ccc4ccccc4c3)cc2)cc1 ; CC1(C)c2ccccc2-c2ccc(N(c3ccc4c(c3)C(C)(C)c3ccccc3-4)c3cc4oc5c6ccccc6c6ccccc6c5c4cc3-c3ccc4ccccc4c3)cc21 ; CC1(C)c2ccccc2-c2ccc(N(c3ccc(-c4ccccc4)cc3)c3ccc(-c4cc5oc6c7ccccc7c7ccccc7c6c5cc4-c4cccc5ccccc45)cc3)cc21]

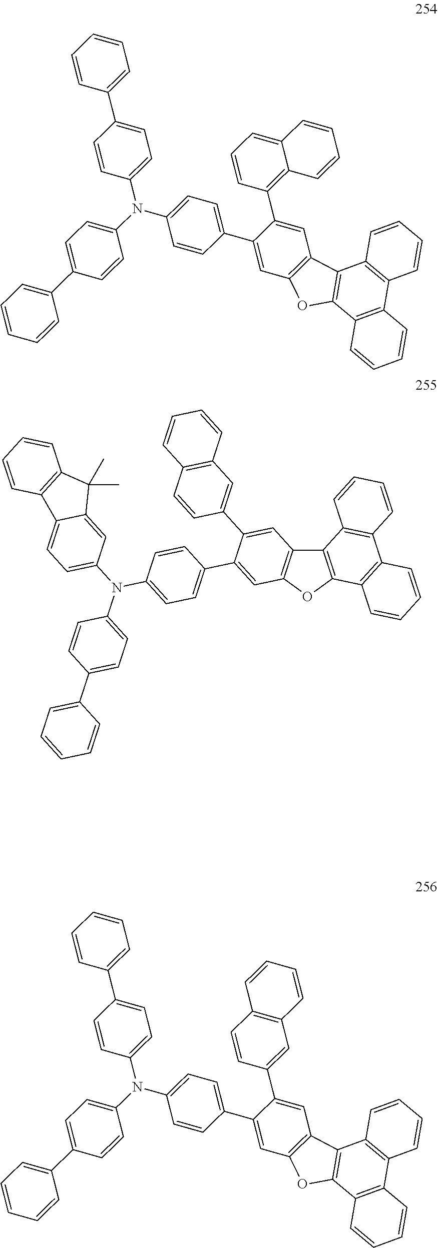 [SMILES: c1ccc(-c2ccc(N(c3ccc(-c4ccccc4)cc3)c3ccc(-c4cc5oc6c7ccccc7c7ccccc7c6c5cc4-c4cccc5ccccc45)cc3)cc2)cc1 ; CC1(C)c2ccccc2-c2ccc(N(c3ccc(-c4ccccc4)cc3)c3ccc(-c4cc5oc6c7ccccc7c7ccccc7c6c5cc4-c4ccc5ccccc5c4)cc3)cc21 ; c1ccc(-c2ccc(N(c3ccc(-c4ccccc4)cc3)c3ccc(-c4cc5oc6c7ccccc7c7ccccc7c6c5cc4-c4ccc5ccccc5c4)cc3)cc2)cc1]

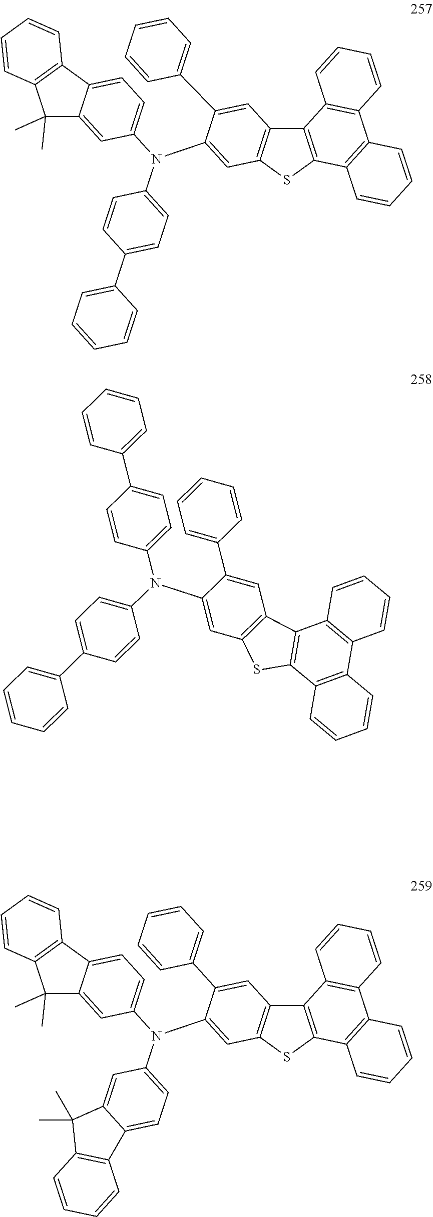

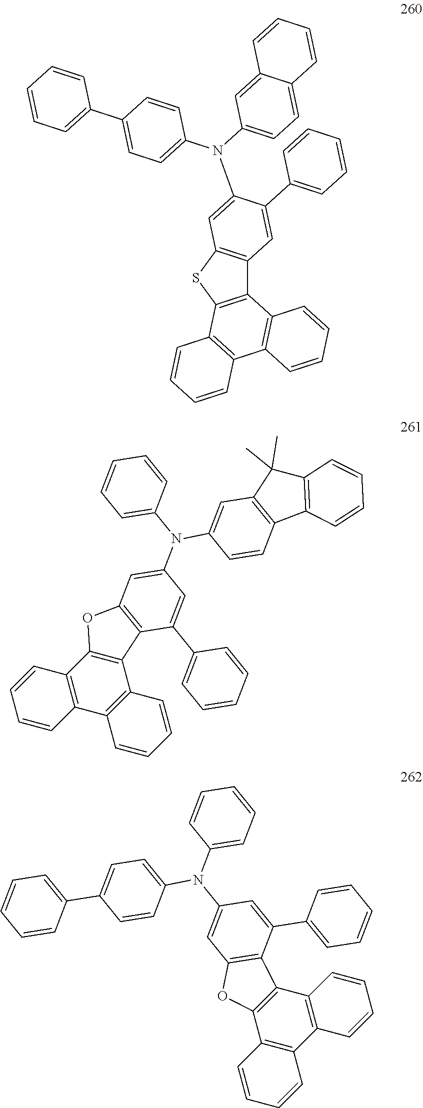 [SMILES: c1ccc(-c2ccc(N(c3ccc4ccccc4c3)c3cc4sc5c6ccccc6c6ccccc6c5c4cc3-c3ccccc3)cc2)cc1 ; CC1(C)c2ccccc2-c2ccc(N(c3ccccc3)c3cc(-c4ccccc4)c4c(c3)oc3c5ccccc5c5ccccc5c34)cc21 ; c1ccc(-c2ccc(N(c3ccccc3)c3cc(-c4ccccc4)c4c(c3)oc3c5ccccc5c5ccccc5c34)cc2)cc1]

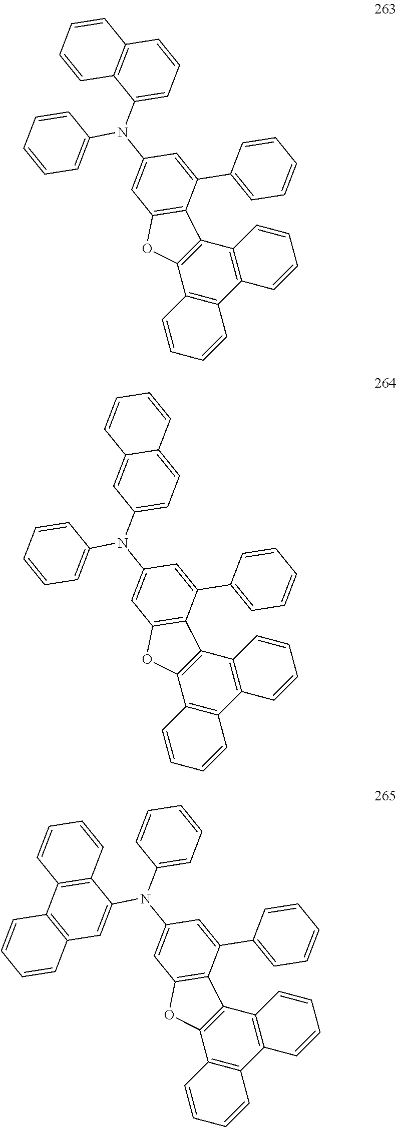

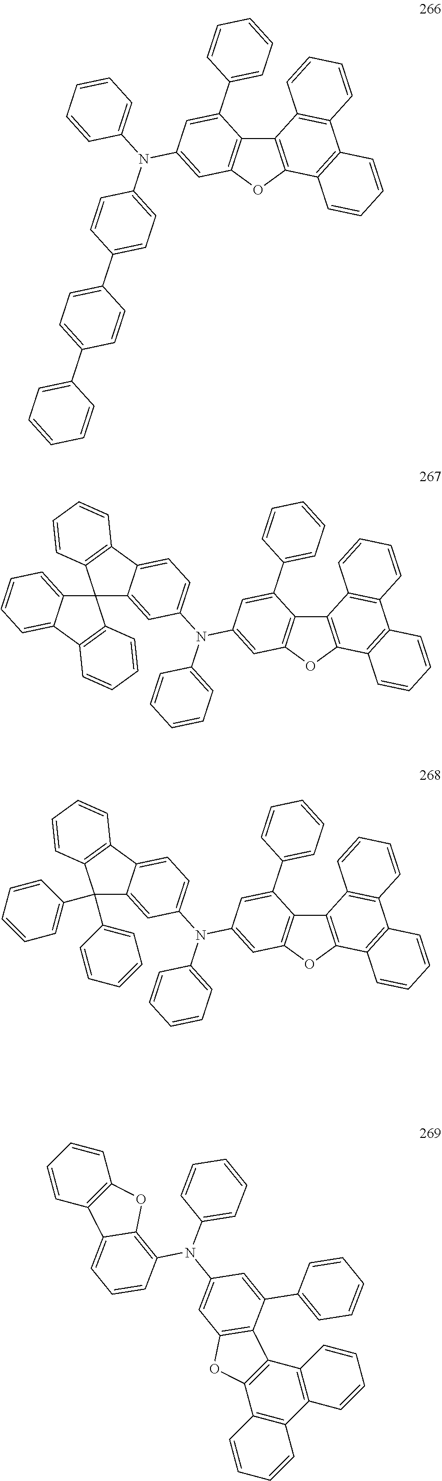 [SMILES: c1ccc(-c2ccc(-c3ccc(N(c4ccccc4)c4cc(-c5ccccc5)c5c(c4)oc4c6ccccc6c6ccccc6c45)cc3)cc2)cc1 ; c1ccc(-c2cc(N(c3ccccc3)c3ccc4c(c3)C3(c5ccccc5-c5ccccc53)c3ccccc3-4)cc3oc4c5ccccc5c5ccccc5c4c23)cc1 ; c1ccc(-c2cc(N(c3ccccc3)c3ccc4c(c3)C(c3ccccc3)(c3ccccc3)c3ccccc3-4)cc3oc4c5ccccc5c5ccccc5c4c23)cc1 ; c1ccc(-c2cc(N(c3ccccc3)c3cccc4c3oc3ccccc34)cc3oc4c5ccccc5c5ccccc5c4c23)cc1]

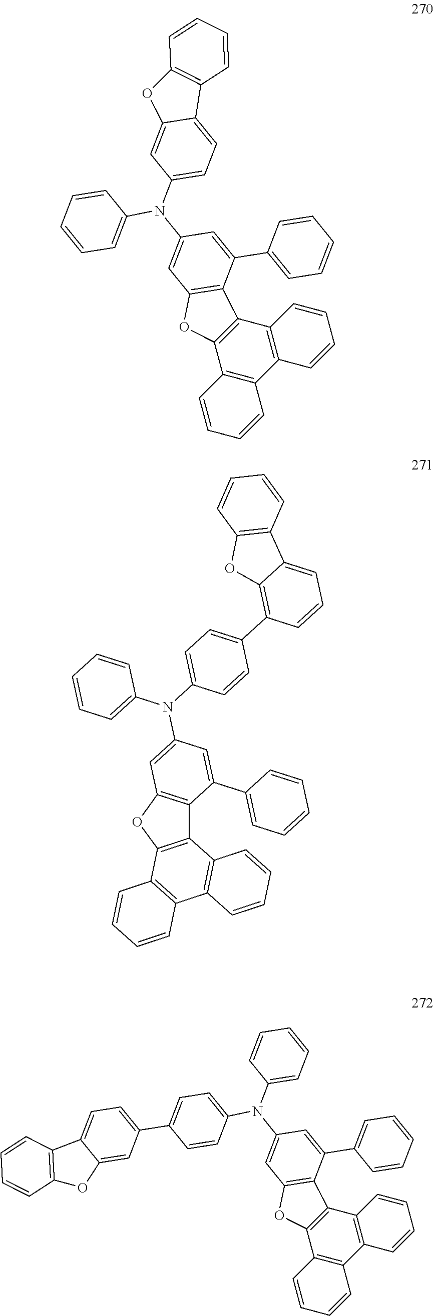

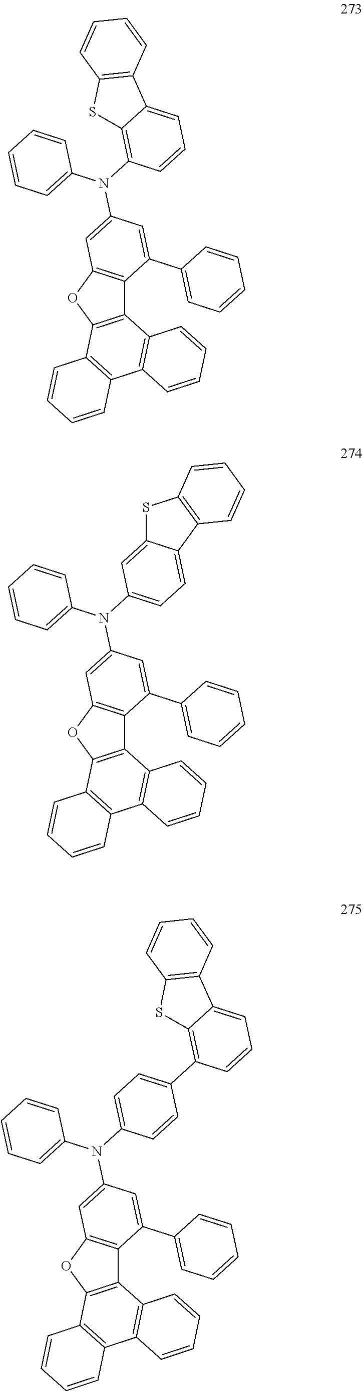 [SMILES: c1ccc(-c2cc(N(c3ccccc3)c3cccc4c3sc3ccccc34)cc3oc4c5ccccc5c5ccccc5c4c23)cc1 ; c1ccc(-c2cc(N(c3ccccc3)c3ccc4c(c3)sc3ccccc34)cc3oc4c5ccccc5c5ccccc5c4c23)cc1 ; c1ccc(-c2cc(N(c3ccccc3)c3ccc(-c4cccc5c4sc4ccccc45)cc3)cc3oc4c5ccccc5c5ccccc5c4c23)cc1]

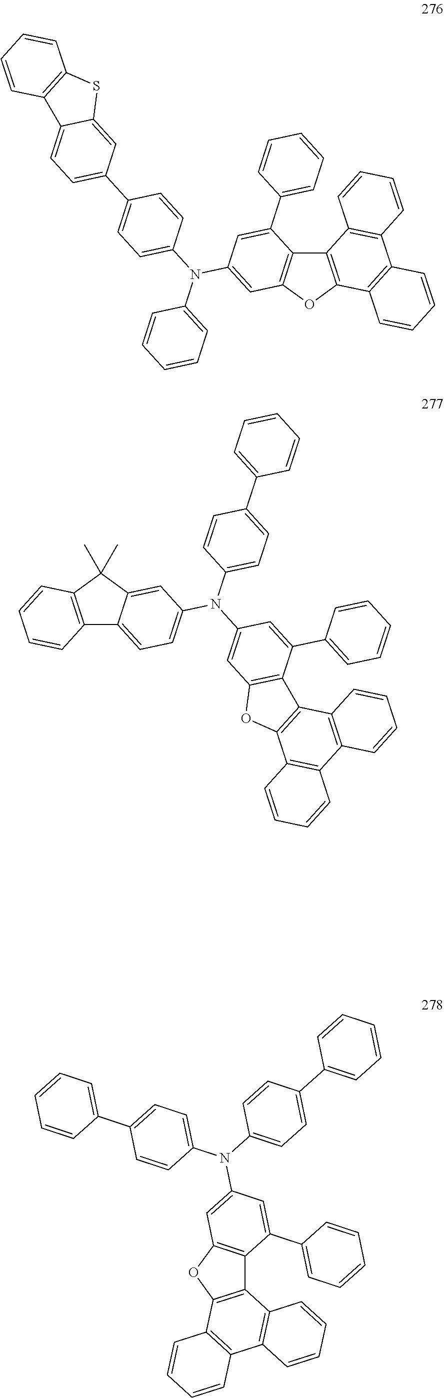 [SMILES: c1ccc(-c2cc(N(c3ccccc3)c3ccc(-c4ccc5c(c4)sc4ccccc45)cc3)cc3oc4c5ccccc5c5ccccc5c4c23)cc1 ; CC1(C)c2ccccc2-c2ccc(N(c3ccc(-c4ccccc4)cc3)c3cc(-c4ccccc4)c4c(c3)oc3c5ccccc5c5ccccc5c34)cc21 ; c1ccc(-c2ccc(N(c3ccc(-c4ccccc4)cc3)c3cc(-c4ccccc4)c4c(c3)oc3c5ccccc5c5ccccc5c34)cc2)cc1]

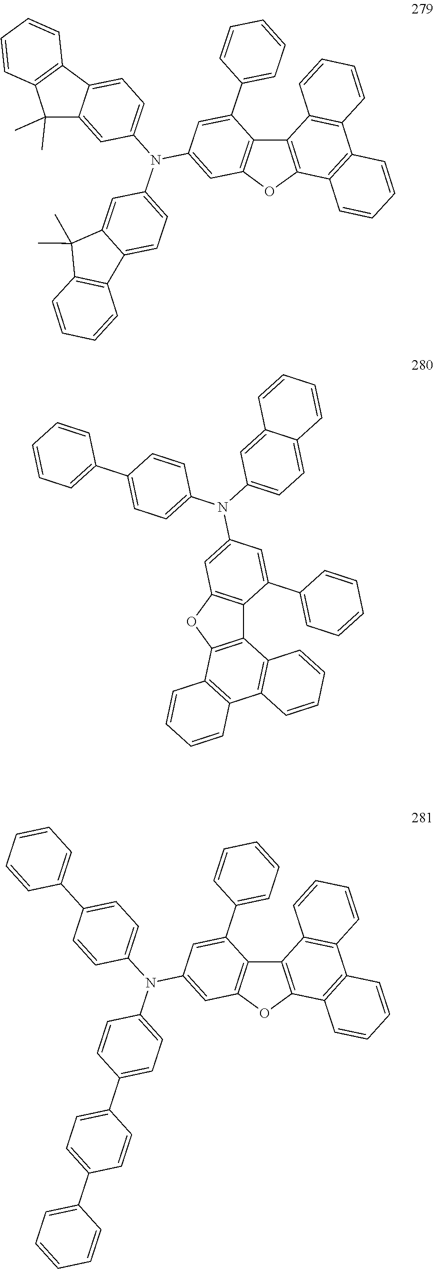 [SMILES: CC1(C)c2ccccc2-c2ccc(N(c3ccc4c(c3)C(C)(C)c3ccccc3-4)c3cc(-c4ccccc4)c4c(c3)oc3c5ccccc5c5ccccc5c34)cc21 ; c1ccc(-c2ccc(N(c3ccc4ccccc4c3)c3cc(-c4ccccc4)c4c(c3)oc3c5ccccc5c5ccccc5c34)cc2)cc1 ; c1ccc(-c2ccc(-c3ccc(N(c4ccc(-c5ccccc5)cc4)c4cc(-c5ccccc5)c5c(c4)oc4c6ccccc6c6ccccc6c45)cc3)cc2)cc1]

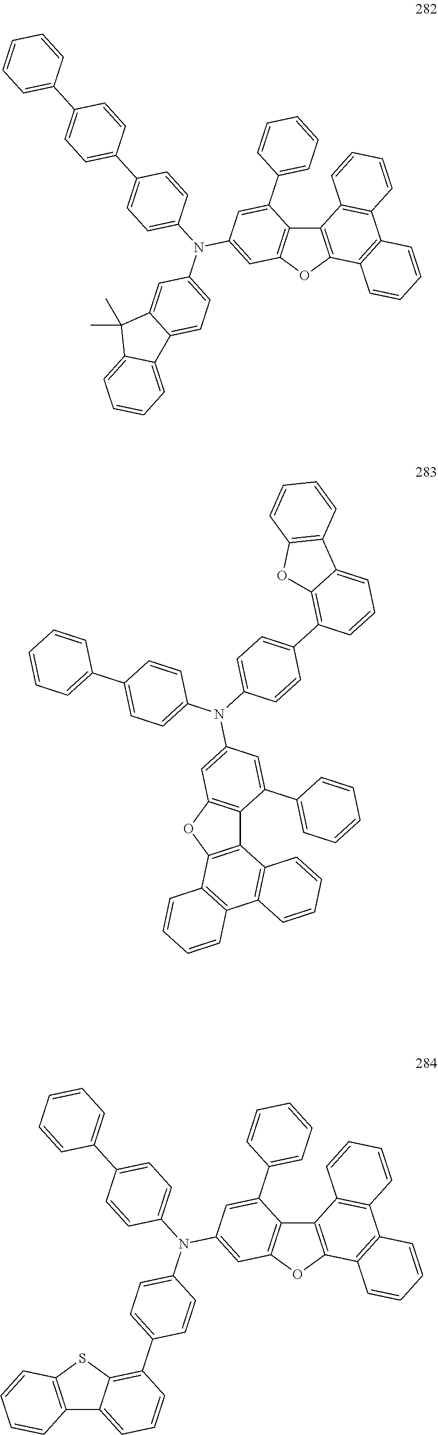 [SMILES: CC1(C)c2ccccc2-c2ccc(N(c3ccc(-c4ccc(-c5ccccc5)cc4)cc3)c3cc(-c4ccccc4)c4c(c3)oc3c5ccccc5c5ccccc5c34)cc21 ; c1ccc(-c2ccc(N(c3ccc(-c4cccc5c4oc4ccccc45)cc3)c3cc(-c4ccccc4)c4c(c3)oc3c5ccccc5c5ccccc5c34)cc2)cc1 ; c1ccc(-c2ccc(N(c3ccc(-c4cccc5c4sc4ccccc45)cc3)c3cc(-c4ccccc4)c4c(c3)oc3c5ccccc5c5ccccc5c34)cc2)cc1]

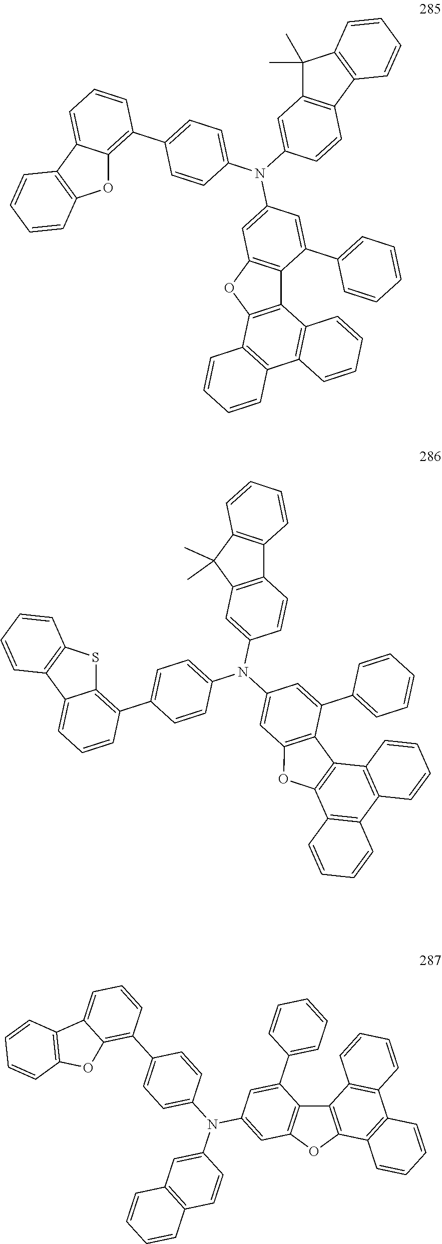 [SMILES: CC1(C)c2ccccc2-c2ccc(N(c3ccc(-c4cccc5c4oc4ccccc45)cc3)c3cc(-c4ccccc4)c4c(c3)oc3c5ccccc5c5ccccc5c34)cc21 ; CC1(C)c2ccccc2-c2ccc(N(c3ccc(-c4cccc5c4sc4ccccc45)cc3)c3cc(-c4ccccc4)c4c(c3)oc3c5ccccc5c5ccccc5c34)cc21 ; c1ccc(-c2cc(N(c3ccc(-c4cccc5c4oc4ccccc45)cc3)c3ccc4ccccc4c3)cc3oc4c5ccccc5c5ccccc5c4c23)cc1]

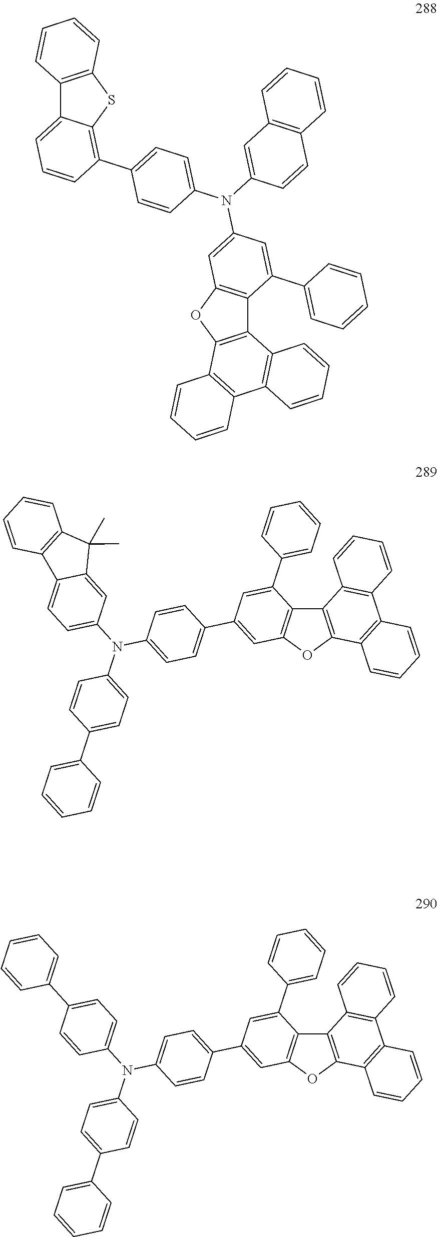 [SMILES: c1ccc(-c2cc(N(c3ccc(-c4cccc5c4sc4ccccc45)cc3)c3ccc4ccccc4c3)cc3oc4c5ccccc5c5ccccc5c4c23)cc1 ; CC1(C)c2ccccc2-c2ccc(N(c3ccc(-c4ccccc4)cc3)c3ccc(-c4cc(-c5ccccc5)c5c(c4)oc4c6ccccc6c6ccccc6c45)cc3)cc21 ; c1ccc(-c2ccc(N(c3ccc(-c4ccccc4)cc3)c3ccc(-c4cc(-c5ccccc5)c5c(c4)oc4c6ccccc6c6ccccc6c45)cc3)cc2)cc1]

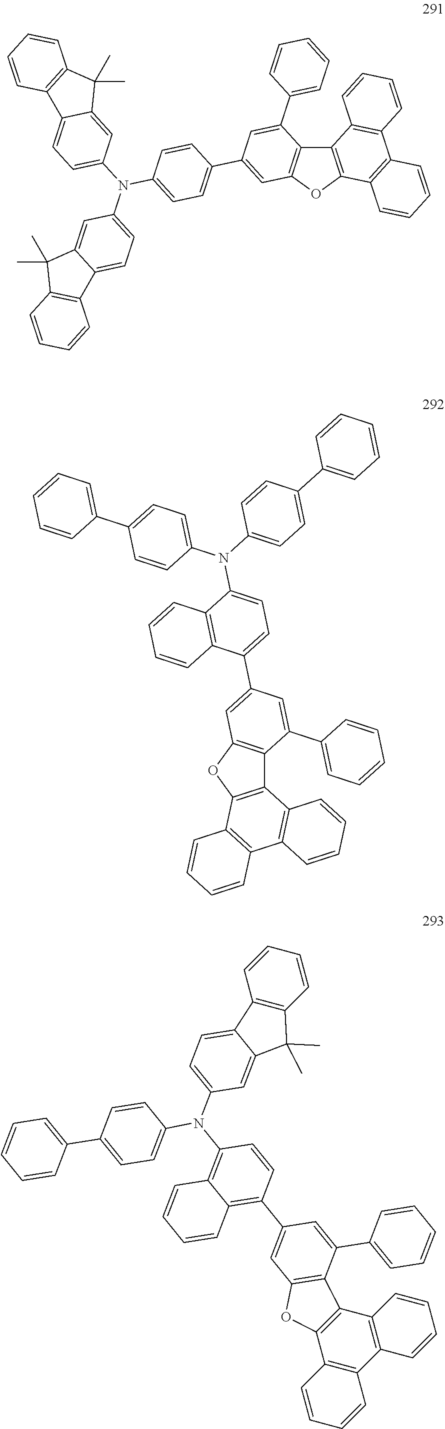 [SMILES: CC1(C)c2ccccc2-c2ccc(N(c3ccc(-c4cc(-c5ccccc5)c5c(c4)oc4c6ccccc6c6ccccc6c45)cc3)c3ccc4c(c3)C(C)(C)c3ccccc3-4)cc21 ; c1ccc(-c2ccc(N(c3ccc(-c4ccccc4)cc3)c3ccc(-c4cc(-c5ccccc5)c5c(c4)oc4c6ccccc6c6ccccc6c45)c4ccccc34)cc2)cc1 ; CC1(C)c2ccccc2-c2ccc(N(c3ccc(-c4ccccc4)cc3)c3ccc(-c4cc(-c5ccccc5)c5c(c4)oc4c6ccccc6c6ccccc6c45)c4ccccc34)cc21]

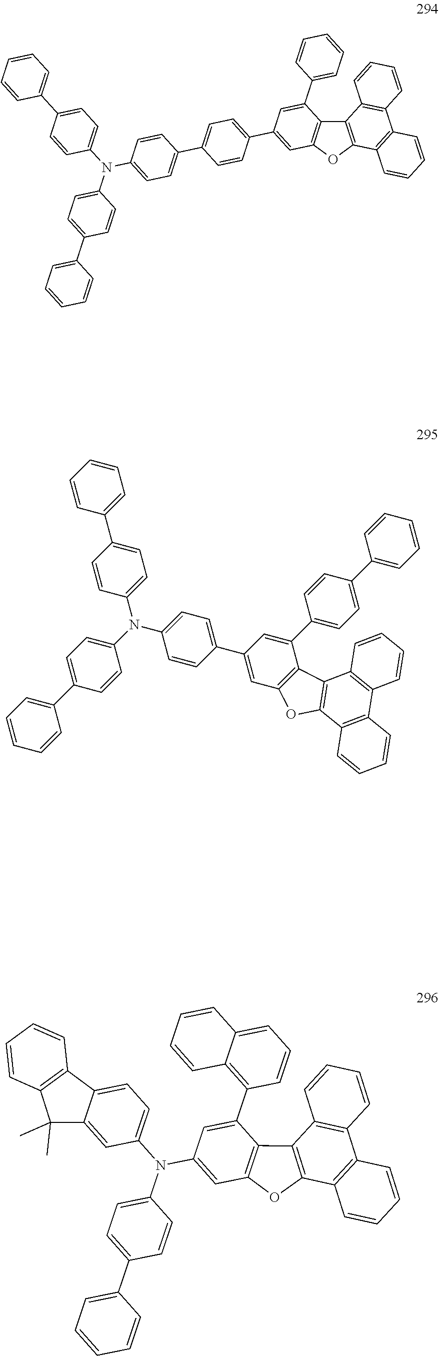 [SMILES: c1ccc(-c2ccc(N(c3ccc(-c4ccccc4)cc3)c3ccc(-c4ccc(-c5cc(-c6ccccc6)c6c(c5)oc5c7ccccc7c7ccccc7c56)cc4)cc3)cc2)cc1 ; c1ccc(-c2ccc(-c3cc(-c4ccc(N(c5ccc(-c6ccccc6)cc5)c5ccc(-c6ccccc6)cc5)cc4)cc4oc5c6ccccc6c6ccccc6c5c34)cc2)cc1 ; CC1(C)c2ccccc2-c2ccc(N(c3ccc(-c4ccccc4)cc3)c3cc(-c4cccc5ccccc45)c4c(c3)oc3c5ccccc5c5ccccc5c34)cc21]

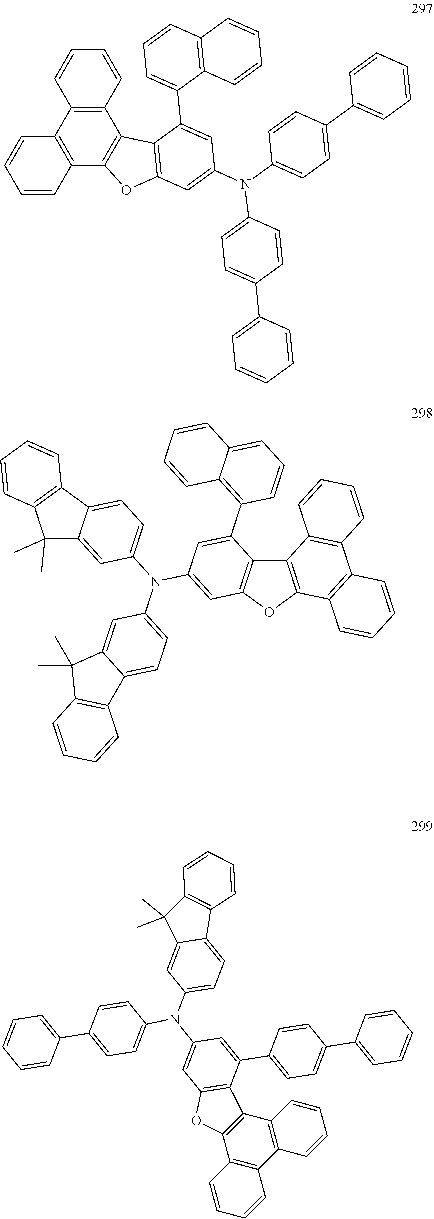 [SMILES: c1ccc(-c2ccc(N(c3ccc(-c4ccccc4)cc3)c3cc(-c4cccc5ccccc45)c4c(c3)oc3c5ccccc5c5ccccc5c34)cc2)cc1 ; CC1(C)c2ccccc2-c2ccc(N(c3ccc4c(c3)C(C)(C)c3ccccc3-4)c3cc(-c4cccc5ccccc45)c4c(c3)oc3c5ccccc5c5ccccc5c34)cc21 ; CC1(C)c2ccccc2-c2ccc(N(c3ccc(-c4ccccc4)cc3)c3cc(-c4ccc(-c5ccccc5)cc4)c4c(c3)oc3c5ccccc5c5ccccc5c34)cc21]

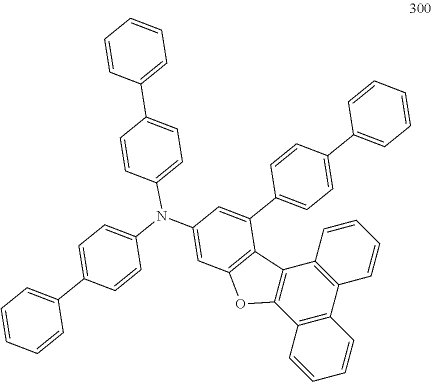 [SMILES: c1ccc(-c2ccc(-c3cc(N(c4ccc(-c5ccccc5)cc4)c4ccc(-c5ccccc5)cc4)cc4oc5c6ccccc6c6ccccc6c5c34)cc2)cc1]

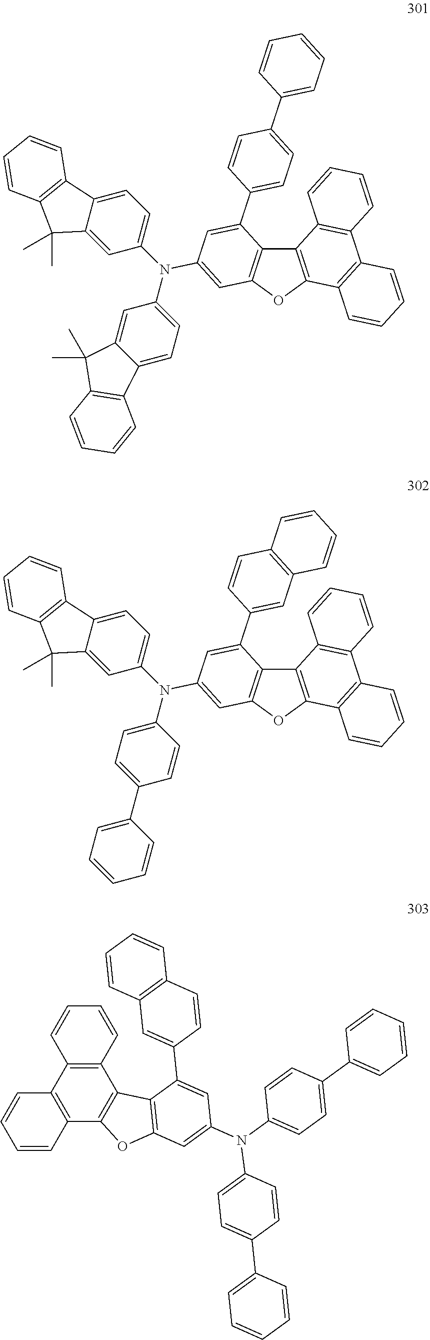 [SMILES: CC1(C)c2ccccc2-c2ccc(N(c3ccc4c(c3)C(C)(C)c3ccccc3-4)c3cc(-c4ccc(-c5ccccc5)cc4)c4c(c3)oc3c5ccccc5c5ccccc5c34)cc21 ; CC1(C)c2ccccc2-c2ccc(N(c3ccc(-c4ccccc4)cc3)c3cc(-c4ccc5ccccc5c4)c4c(c3)oc3c5ccccc5c5ccccc5c34)cc21 ; c1ccc(-c2ccc(N(c3ccc(-c4ccccc4)cc3)c3cc(-c4ccc5ccccc5c4)c4c(c3)oc3c5ccccc5c5ccccc5c34)cc2)cc1]

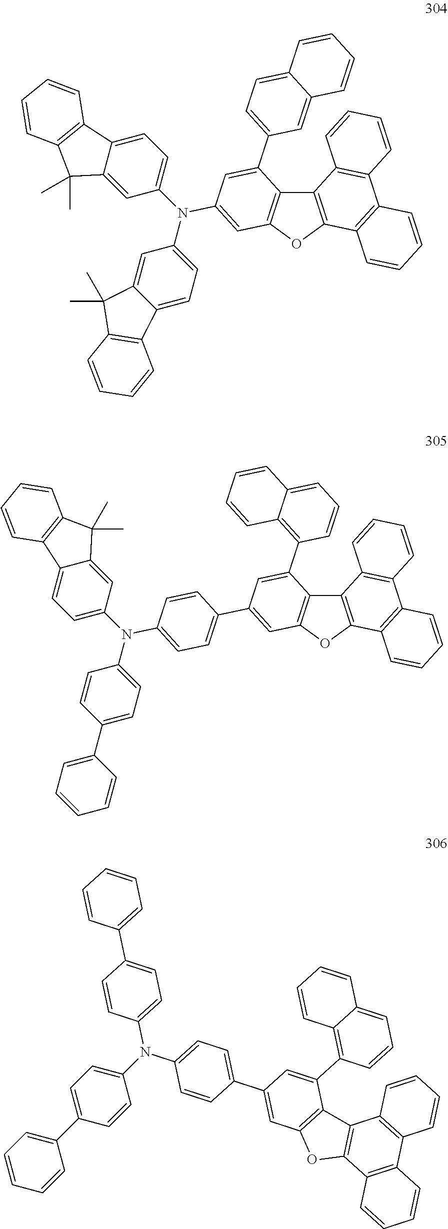 [SMILES: CC1(C)c2ccccc2-c2ccc(N(c3ccc4c(c3)C(C)(C)c3ccccc3-4)c3cc(-c4ccc5ccccc5c4)c4c(c3)oc3c5ccccc5c5ccccc5c34)cc21 ; CC1(C)c2ccccc2-c2ccc(N(c3ccc(-c4ccccc4)cc3)c3ccc(-c4cc(-c5cccc6ccccc56)c5c(c4)oc4c6ccccc6c6ccccc6c45)cc3)cc21 ; c1ccc(-c2ccc(N(c3ccc(-c4ccccc4)cc3)c3ccc(-c4cc(-c5cccc6ccccc56)c5c(c4)oc4c6ccccc6c6ccccc6c45)cc3)cc2)cc1]

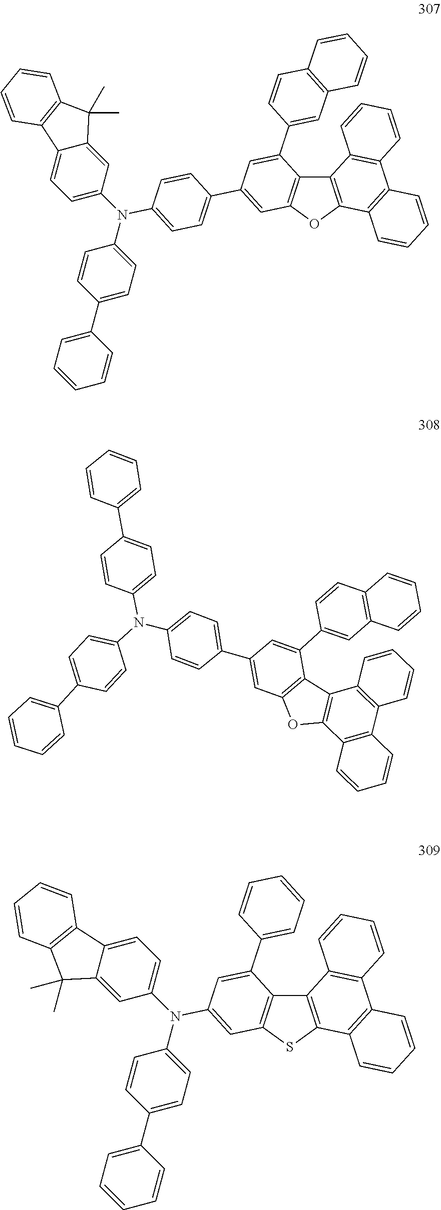 [SMILES: CC1(C)c2ccccc2-c2ccc(N(c3ccc(-c4ccccc4)cc3)c3ccc(-c4cc(-c5ccc6ccccc6c5)c5c(c4)oc4c6ccccc6c6ccccc6c45)cc3)cc21 ; c1ccc(-c2ccc(N(c3ccc(-c4ccccc4)cc3)c3ccc(-c4cc(-c5ccc6ccccc6c5)c5c(c4)oc4c6ccccc6c6ccccc6c45)cc3)cc2)cc1 ; CC1(C)c2ccccc2-c2ccc(N(c3ccc(-c4ccccc4)cc3)c3cc(-c4ccccc4)c4c(c3)sc3c5ccccc5c5ccccc5c34)cc21]

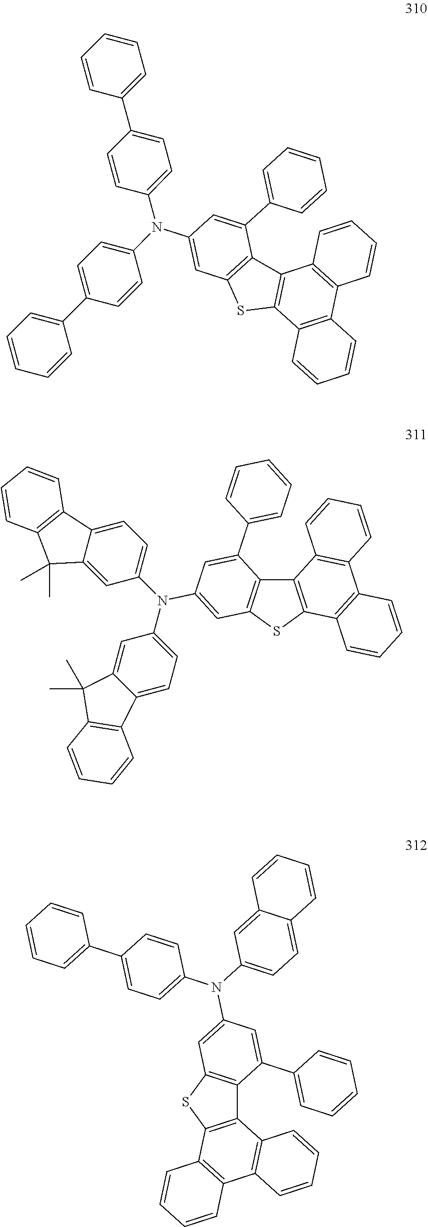 [SMILES: c1ccc(-c2ccc(N(c3ccc(-c4ccccc4)cc3)c3cc(-c4ccccc4)c4c(c3)sc3c5ccccc5c5ccccc5c34)cc2)cc1 ; CC1(C)c2ccccc2-c2ccc(N(c3ccc4c(c3)C(C)(C)c3ccccc3-4)c3cc(-c4ccccc4)c4c(c3)sc3c5ccccc5c5ccccc5c34)cc21 ; c1ccc(-c2ccc(N(c3ccc4ccccc4c3)c3cc(-c4ccccc4)c4c(c3)sc3c5ccccc5c5ccccc5c34)cc2)cc1]

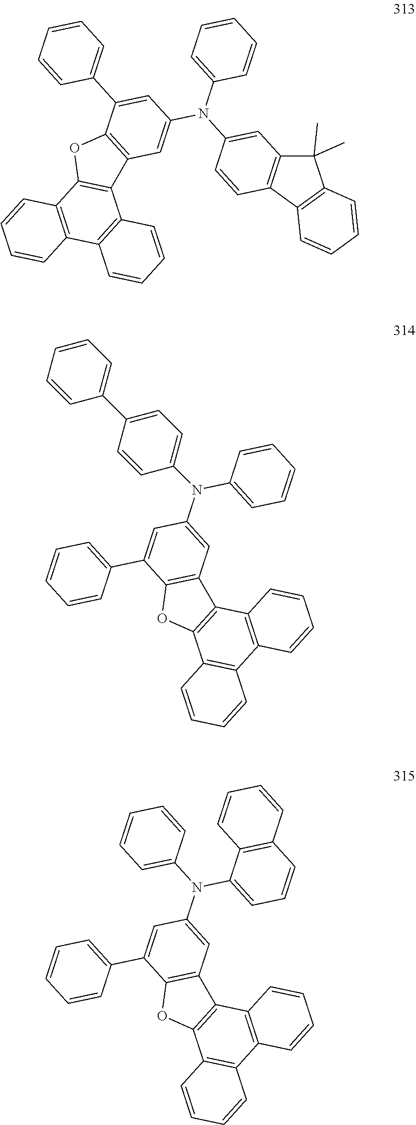 [SMILES: CC1(C)c2ccccc2-c2ccc(N(c3ccccc3)c3cc(-c4ccccc4)c4oc5c6ccccc6c6ccccc6c5c4c3)cc21 ; c1ccc(-c2ccc(N(c3ccccc3)c3cc(-c4ccccc4)c4oc5c6ccccc6c6ccccc6c5c4c3)cc2)cc1 ; c1ccc(-c2cc(N(c3ccccc3)c3cccc4ccccc34)cc3c2oc2c4ccccc4c4ccccc4c32)cc1]

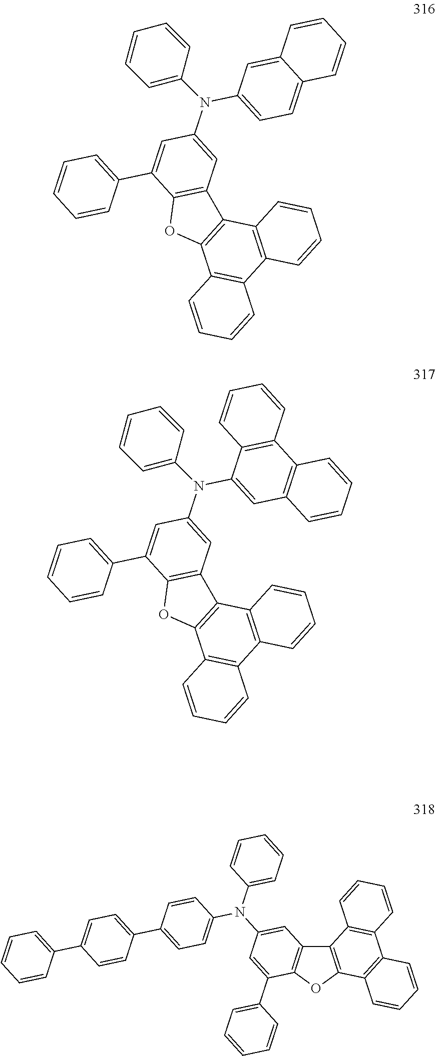 [SMILES: c1ccc(-c2cc(N(c3ccccc3)c3ccc4ccccc4c3)cc3c2oc2c4ccccc4c4ccccc4c32)cc1 ; c1ccc(-c2cc(N(c3ccccc3)c3cc4ccccc4c4ccccc34)cc3c2oc2c4ccccc4c4ccccc4c32)cc1 ; c1ccc(-c2ccc(-c3ccc(N(c4ccccc4)c4cc(-c5ccccc5)c5oc6c7ccccc7c7ccccc7c6c5c4)cc3)cc2)cc1]

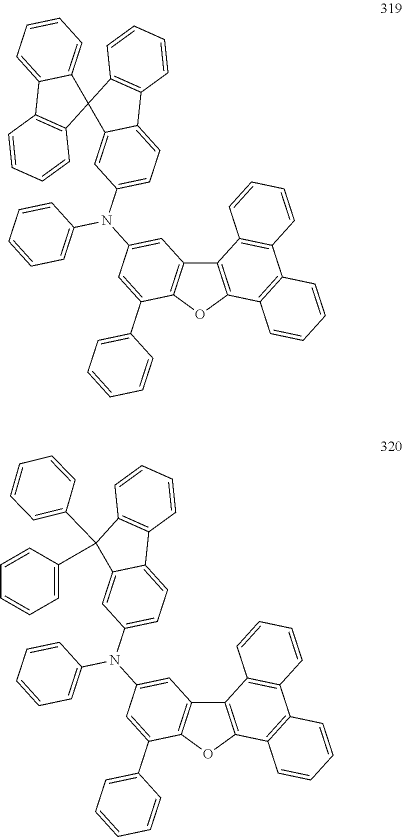 [SMILES: c1ccc(-c2cc(N(c3ccccc3)c3ccc4c(c3)C3(c5ccccc5-c5ccccc53)c3ccccc3-4)cc3c2oc2c4ccccc4c4ccccc4c32)cc1 ; c1ccc(-c2cc(N(c3ccccc3)c3ccc4c(c3)C(c3ccccc3)(c3ccccc3)c3ccccc3-4)cc3c2oc2c4ccccc4c4ccccc4c32)cc1]

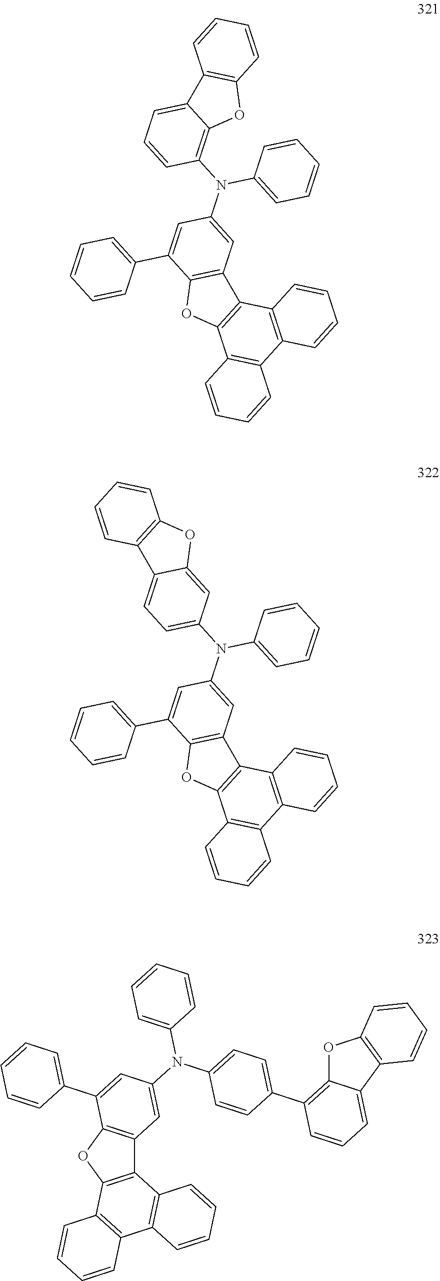 [SMILES: c1ccc(-c2cc(N(c3ccccc3)c3cccc4c3oc3ccccc34)cc3c2oc2c4ccccc4c4ccccc4c32)cc1 ; c1ccc(-c2cc(N(c3ccccc3)c3ccc4c(c3)oc3ccccc34)cc3c2oc2c4ccccc4c4ccccc4c32)cc1 ; c1ccc(-c2cc(N(c3ccccc3)c3ccc(-c4cccc5c4oc4ccccc45)cc3)cc3c2oc2c4ccccc4c4ccccc4c32)cc1]

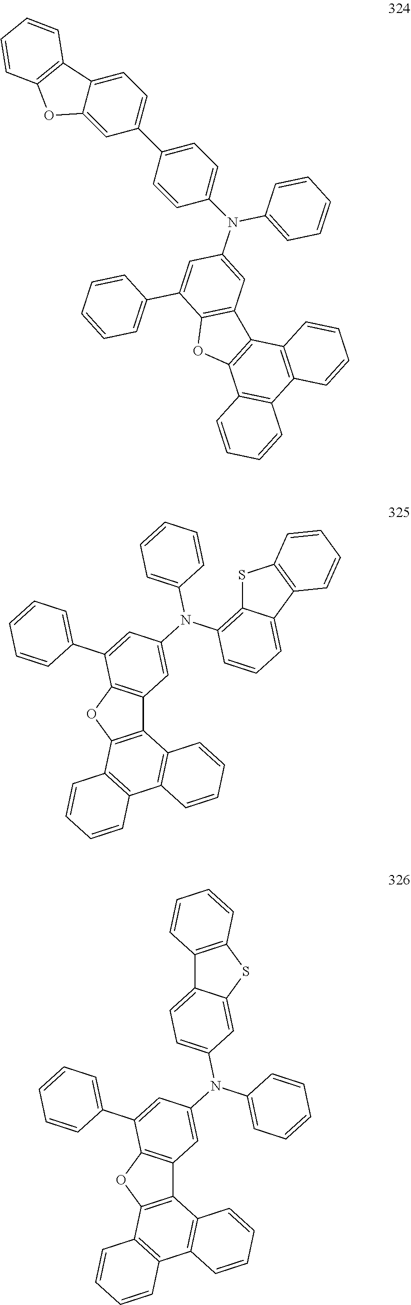 [SMILES: c1ccc(-c2cc(N(c3ccccc3)c3ccc(-c4ccc5c(c4)oc4ccccc45)cc3)cc3c2oc2c4ccccc4c4ccccc4c32)cc1 ; c1ccc(-c2cc(N(c3ccccc3)c3cccc4c3sc3ccccc34)cc3c2oc2c4ccccc4c4ccccc4c32)cc1 ; c1ccc(-c2cc(N(c3ccccc3)c3ccc4c(c3)sc3ccccc34)cc3c2oc2c4ccccc4c4ccccc4c32)cc1]

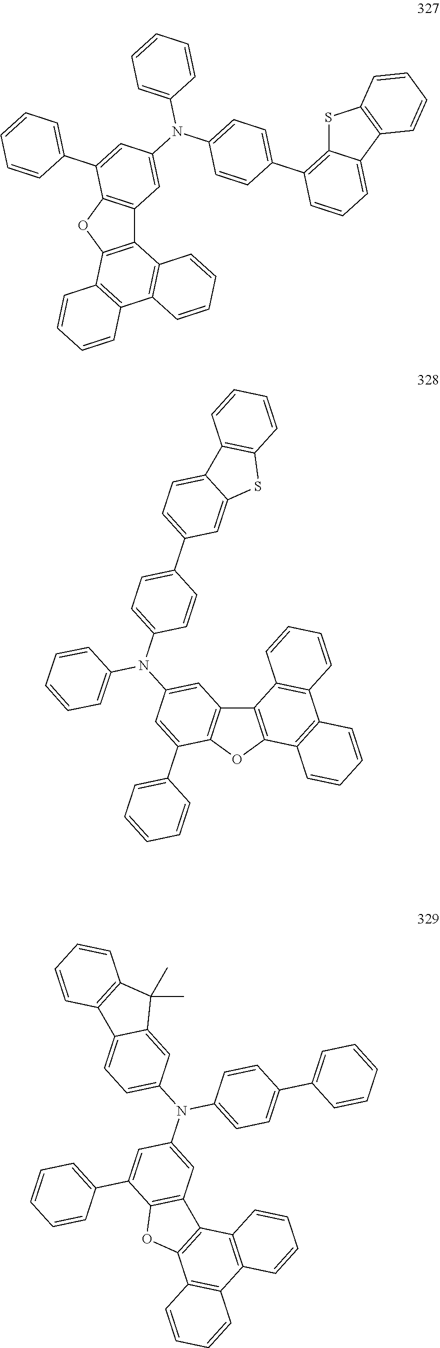 [SMILES: c1ccc(-c2cc(N(c3ccccc3)c3ccc(-c4cccc5c4sc4ccccc45)cc3)cc3c2oc2c4ccccc4c4ccccc4c32)cc1 ; c1ccc(-c2cc(N(c3ccccc3)c3ccc(-c4ccc5c(c4)sc4ccccc45)cc3)cc3c2oc2c4ccccc4c4ccccc4c32)cc1 ; CC1(C)c2ccccc2-c2ccc(N(c3ccc(-c4ccccc4)cc3)c3cc(-c4ccccc4)c4oc5c6ccccc6c6ccccc6c5c4c3)cc21]

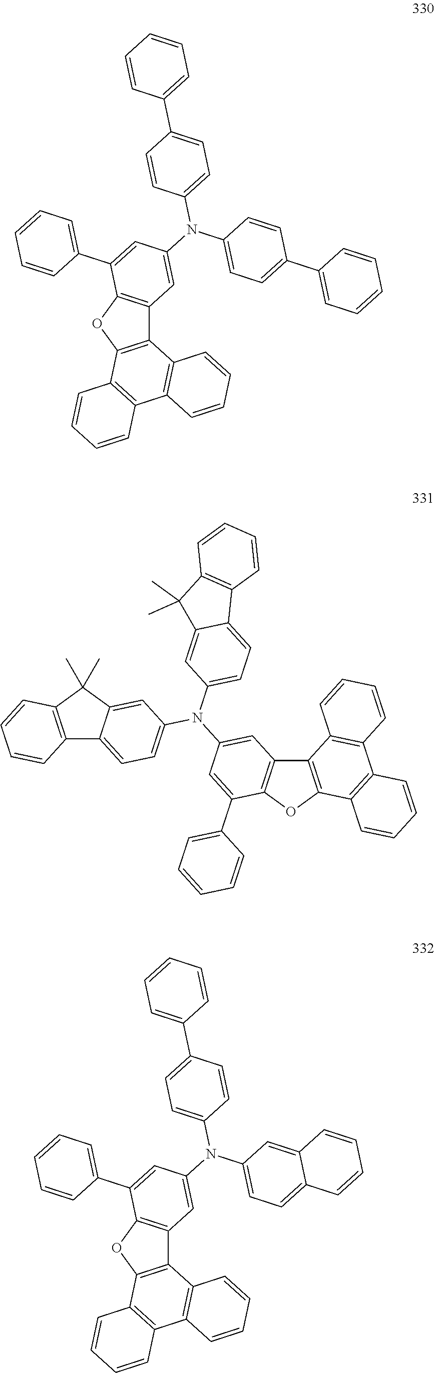 [SMILES: c1ccc(-c2ccc(N(c3ccc(-c4ccccc4)cc3)c3cc(-c4ccccc4)c4oc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccccc4)c4oc5c6ccccc6c6ccccc6c5c4c3)cc21 ; c1ccc(-c2ccc(N(c3ccc4ccccc4c3)c3cc(-c4ccccc4)c4oc5c6ccccc6c6ccccc6c5c4c3)cc2)cc1]

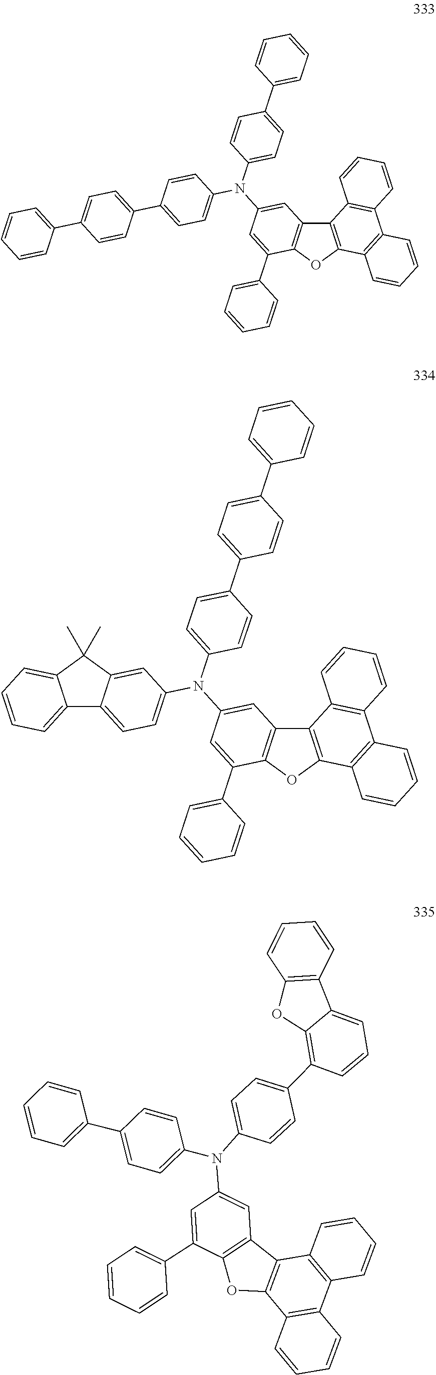 [SMILES: c1ccc(-c2ccc(-c3ccc(N(c4ccc(-c5ccccc5)cc4)c4cc(-c5ccccc5)c5oc6c7ccccc7c7ccccc7c6c5c4)cc3)cc2)cc1 ; CC1(C)c2ccccc2-c2ccc(N(c3ccc(-c4ccc(-c5ccccc5)cc4)cc3)c3cc(-c4ccccc4)c4oc5c6ccccc6c6ccccc6c5c4c3)cc21 ; c1ccc(-c2ccc(N(c3ccc(-c4cccc5c4oc4ccccc45)cc3)c3cc(-c4ccccc4)c4oc5c6ccccc6c6ccccc6c5c4c3)cc2)cc1]

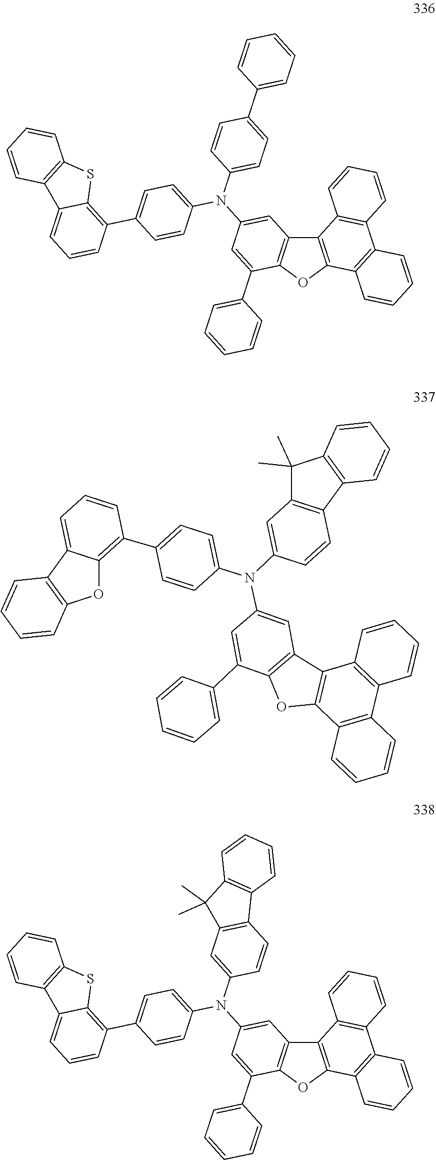 [SMILES: c1ccc(-c2ccc(N(c3ccc(-c4cccc5c4sc4ccccc45)cc3)c3cc(-c4ccccc4)c4oc5c6ccccc6c6ccccc6c5c4c3)cc2)cc1 ; CC1(C)c2ccccc2-c2ccc(N(c3ccc(-c4cccc5c4oc4ccccc45)cc3)c3cc(-c4ccccc4)c4oc5c6ccccc6c6ccccc6c5c4c3)cc21 ; CC1(C)c2ccccc2-c2ccc(N(c3ccc(-c4cccc5c4sc4ccccc45)cc3)c3cc(-c4ccccc4)c4oc5c6ccccc6c6ccccc6c5c4c3)cc21]

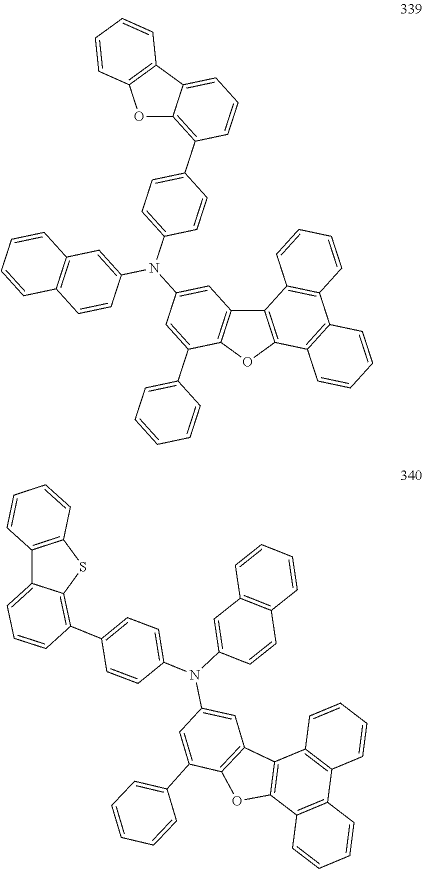 [SMILES: c1ccc(-c2cc(N(c3ccc(-c4cccc5c4oc4ccccc45)cc3)c3ccc4ccccc4c3)cc3c2oc2c4ccccc4c4ccccc4c32)cc1 ; c1ccc(-c2cc(N(c3ccc(-c4cccc5c4sc4ccccc45)cc3)c3ccc4ccccc4c3)cc3c2oc2c4ccccc4c4ccccc4c32)cc1]

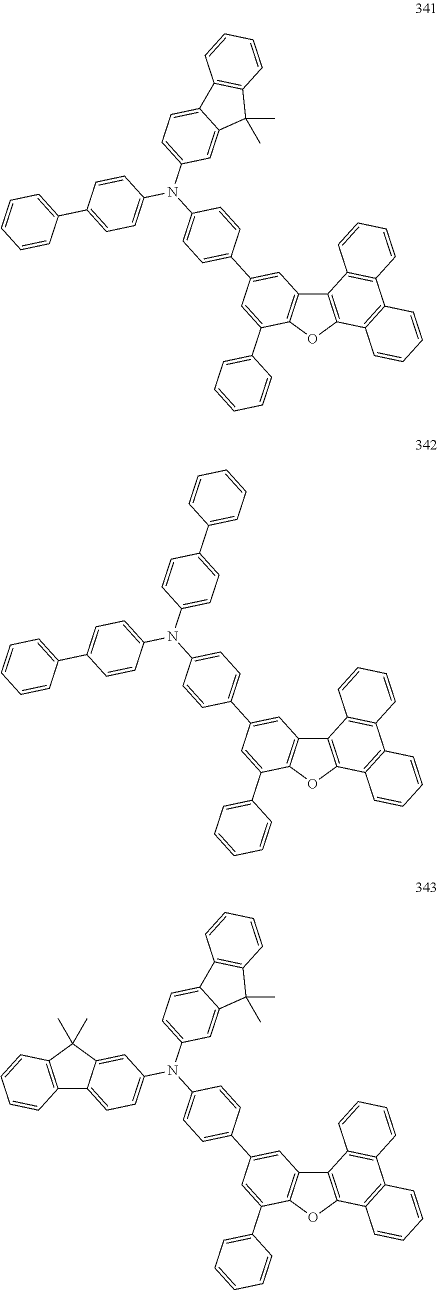 [SMILES: CC1(C)c2ccccc2-c2ccc(N(c3ccc(-c4ccccc4)cc3)c3ccc(-c4cc(-c5ccccc5)c5oc6c7ccccc7c7ccccc7c6c5c4)cc3)cc21 ; c1ccc(-c2ccc(N(c3ccc(-c4ccccc4)cc3)c3ccc(-c4cc(-c5ccccc5)c5oc6c7ccccc7c7ccccc7c6c5c4)cc3)cc2)cc1 ; CC1(C)c2ccccc2-c2ccc(N(c3ccc(-c4cc(-c5ccccc5)c5oc6c7ccccc7c7ccccc7c6c5c4)cc3)c3ccc4c(c3)C(C)(C)c3ccccc3-4)cc21]

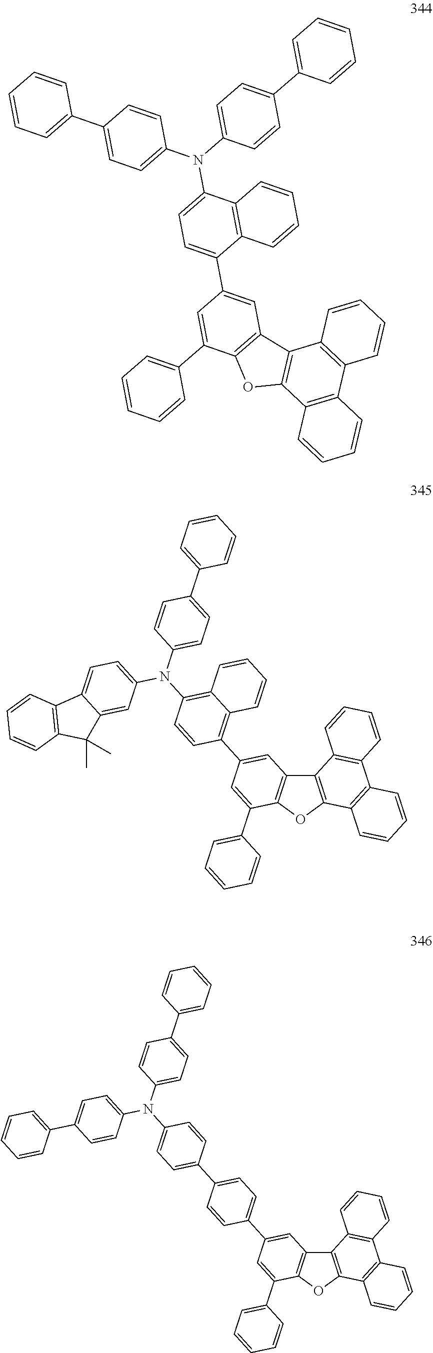 [SMILES: c1ccc(-c2ccc(N(c3ccc(-c4ccccc4)cc3)c3ccc(-c4cc(-c5ccccc5)c5oc6c7ccccc7c7ccccc7c6c5c4)c4ccccc34)cc2)cc1 ; CC1(C)c2ccccc2-c2ccc(N(c3ccc(-c4ccccc4)cc3)c3ccc(-c4cc(-c5ccccc5)c5oc6c7ccccc7c7ccccc7c6c5c4)c4ccccc34)cc21 ; c1ccc(-c2ccc(N(c3ccc(-c4ccccc4)cc3)c3ccc(-c4ccc(-c5cc(-c6ccccc6)c6oc7c8ccccc8c8ccccc8c7c6c5)cc4)cc3)cc2)cc1]

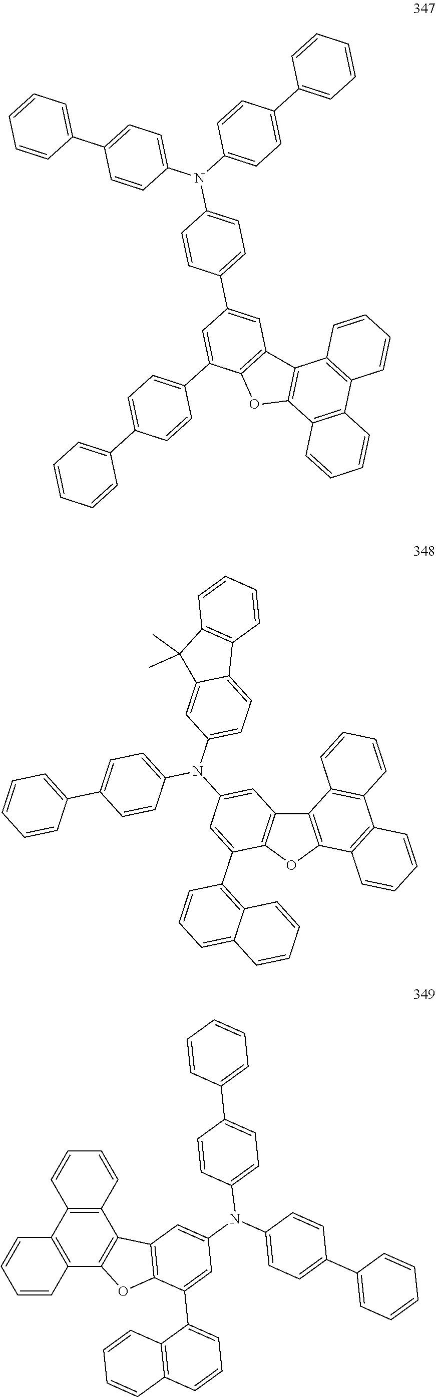 [SMILES: c1ccc(-c2ccc(-c3cc(-c4ccc(N(c5ccc(-c6ccccc6)cc5)c5ccc(-c6ccccc6)cc5)cc4)cc4c3oc3c5ccccc5c5ccccc5c43)cc2)cc1 ; CC1(C)c2ccccc2-c2ccc(N(c3ccc(-c4ccccc4)cc3)c3cc(-c4cccc5ccccc45)c4oc5c6ccccc6c6ccccc6c5c4c3)cc21 ; c1ccc(-c2ccc(N(c3ccc(-c4ccccc4)cc3)c3cc(-c4cccc5ccccc45)c4oc5c6ccccc6c6ccccc6c5c4c3)cc2)cc1]

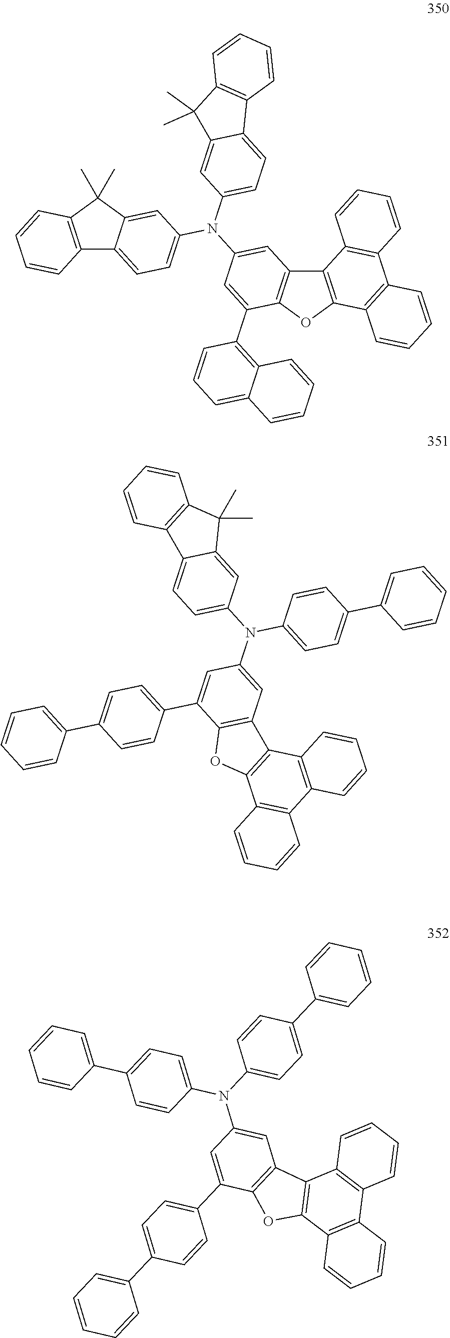 [SMILES: CC1(C)c2ccccc2-c2ccc(N(c3ccc4c(c3)C(C)(C)c3ccccc3-4)c3cc(-c4cccc5ccccc45)c4oc5c6ccccc6c6ccccc6c5c4c3)cc21 ; CC1(C)c2ccccc2-c2ccc(N(c3ccc(-c4ccccc4)cc3)c3cc(-c4ccc(-c5ccccc5)cc4)c4oc5c6ccccc6c6ccccc6c5c4c3)cc21 ; c1ccc(-c2ccc(-c3cc(N(c4ccc(-c5ccccc5)cc4)c4ccc(-c5ccccc5)cc4)cc4c3oc3c5ccccc5c5ccccc5c43)cc2)cc1]

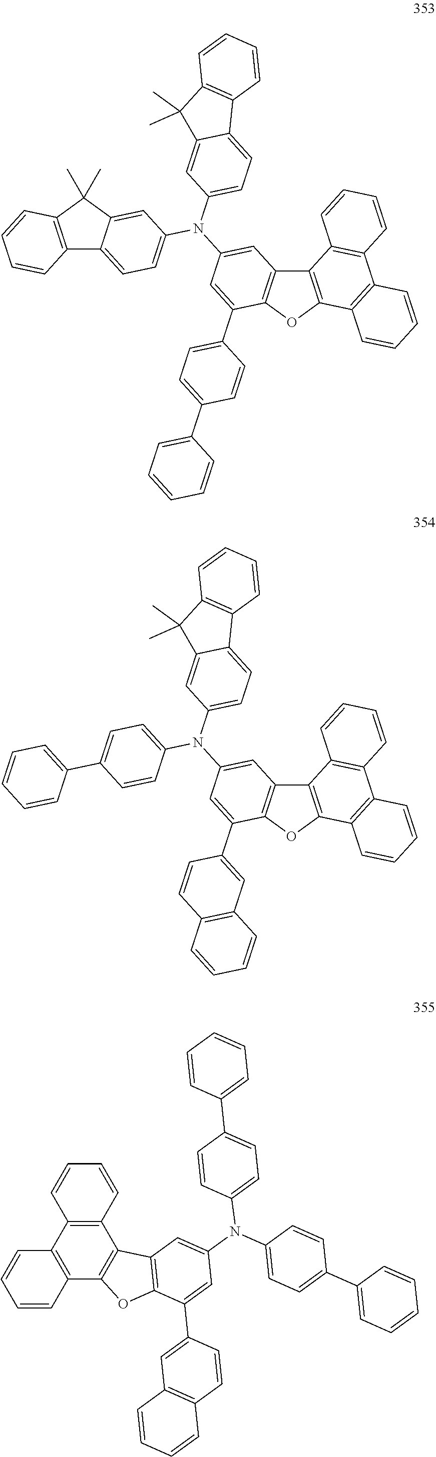 [SMILES: CC1(C)c2ccccc2-c2ccc(N(c3ccc4c(c3)C(C)(C)c3ccccc3-4)c3cc(-c4ccc(-c5ccccc5)cc4)c4oc5c6ccccc6c6ccccc6c5c4c3)cc21 ; CC1(C)c2ccccc2-c2ccc(N(c3ccc(-c4ccccc4)cc3)c3cc(-c4ccc5ccccc5c4)c4oc5c6ccccc6c6ccccc6c5c4c3)cc21 ; c1ccc(-c2ccc(N(c3ccc(-c4ccccc4)cc3)c3cc(-c4ccc5ccccc5c4)c4oc5c6ccccc6c6ccccc6c5c4c3)cc2)cc1]

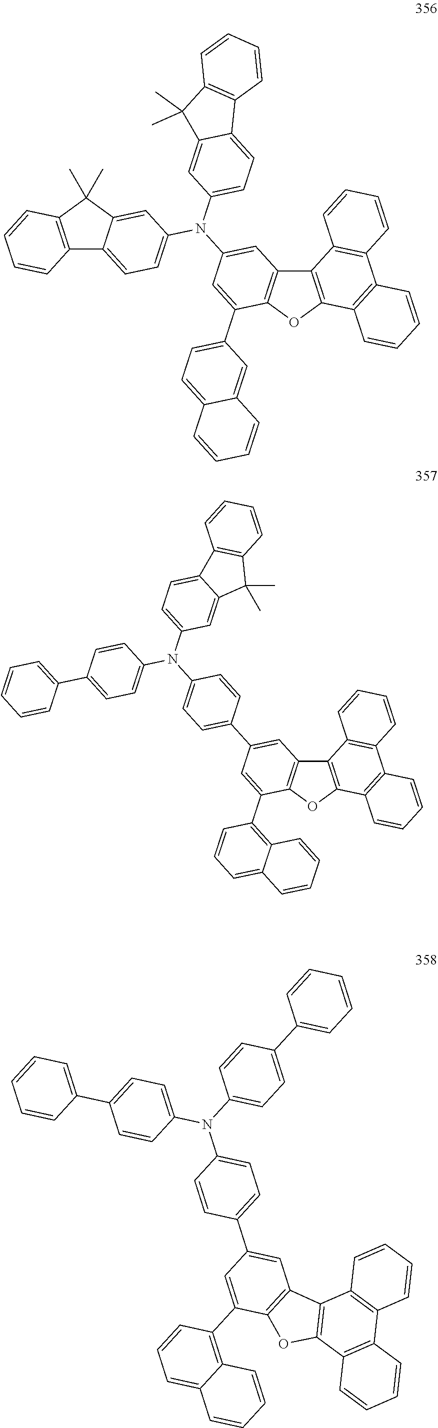

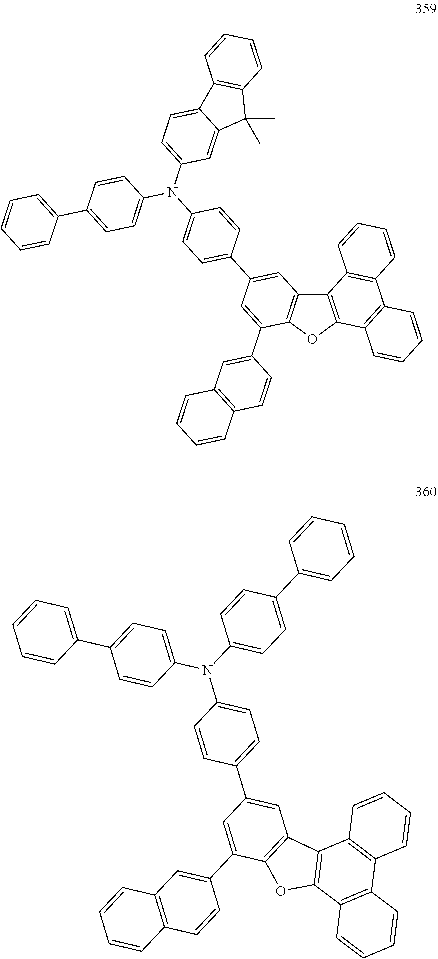 [SMILES: CC1(C)c2ccccc2-c2ccc(N(c3ccc(-c4ccccc4)cc3)c3ccc(-c4cc(-c5ccc6ccccc6c5)c5oc6c7ccccc7c7ccccc7c6c5c4)cc3)cc21 ; c1ccc(-c2ccc(N(c3ccc(-c4ccccc4)cc3)c3ccc(-c4cc(-c5ccc6ccccc6c5)c5oc6c7ccccc7c7ccccc7c6c5c4)cc3)cc2)cc1]

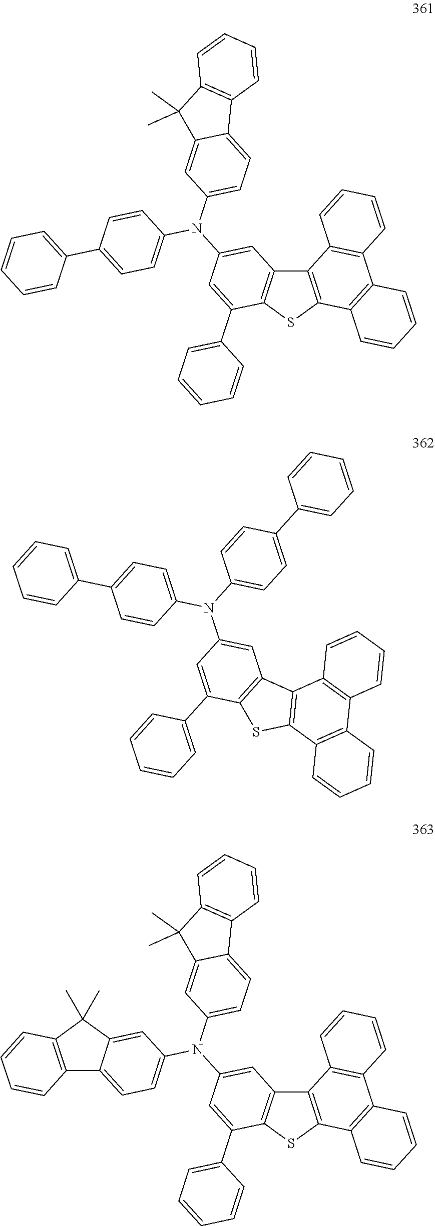 [SMILES: CC1(C)c2ccccc2-c2ccc(N(c3ccc(-c4ccccc4)cc3)c3cc(-c4ccccc4)c4sc5c6ccccc6c6ccccc6c5c4c3)cc21 ; c1ccc(-c2ccc(N(c3ccc(-c4ccccc4)cc3)c3cc(-c4ccccc4)c4sc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccccc4)c4sc5c6ccccc6c6ccccc6c5c4c3)cc21]

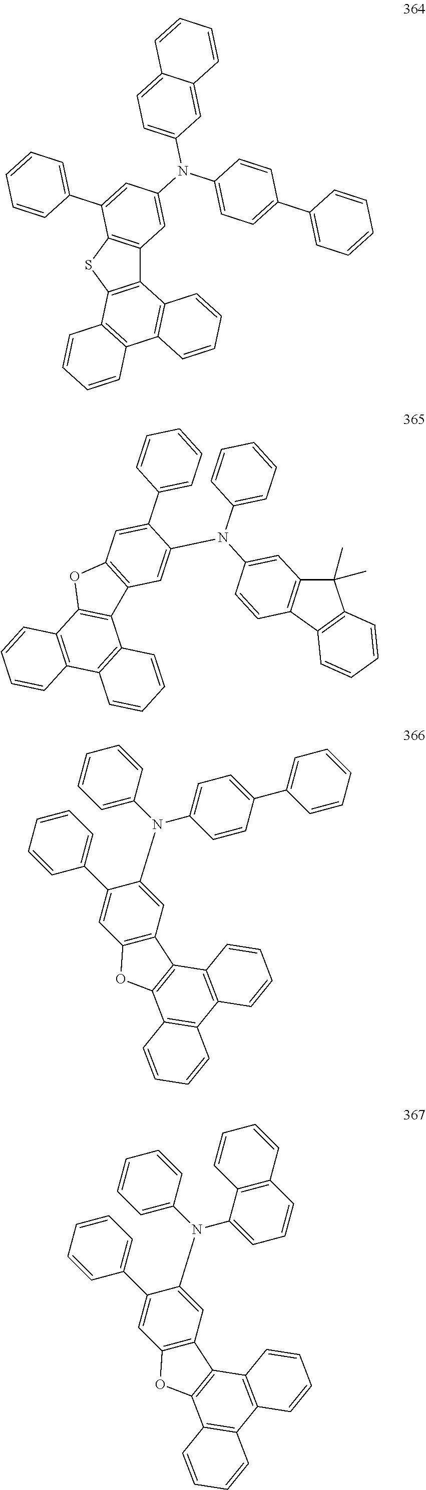

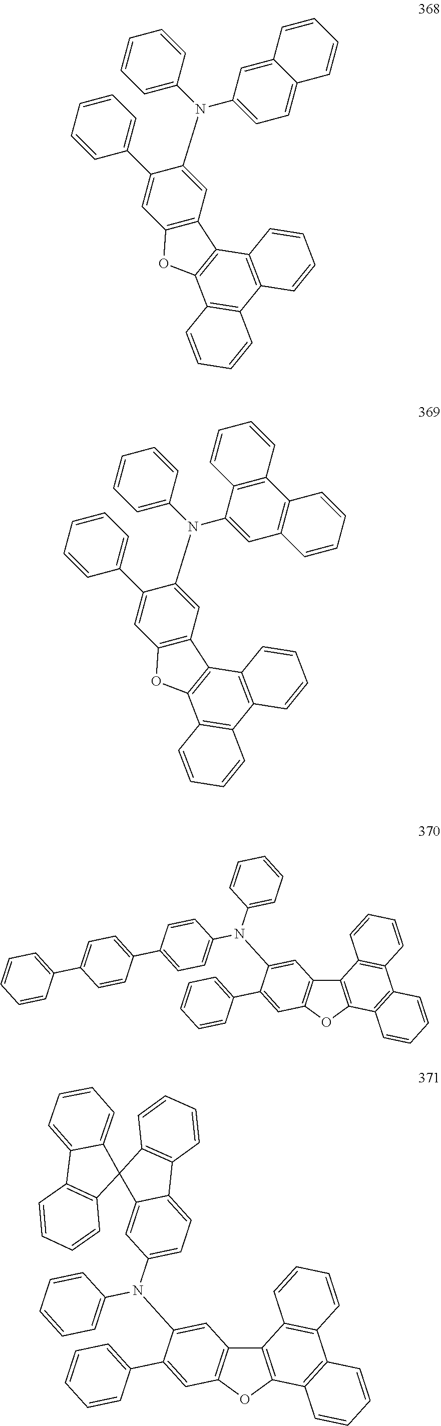 [SMILES: c1ccc(-c2cc3oc4c5ccccc5c5ccccc5c4c3cc2N(c2ccccc2)c2ccc3ccccc3c2)cc1 ; c1ccc(-c2cc3oc4c5ccccc5c5ccccc5c4c3cc2N(c2ccccc2)c2cc3ccccc3c3ccccc23)cc1 ; c1ccc(-c2ccc(-c3ccc(N(c4ccccc4)c4cc5c(cc4-c4ccccc4)oc4c6ccccc6c6ccccc6c54)cc3)cc2)cc1 ; c1ccc(-c2cc3oc4c5ccccc5c5ccccc5c4c3cc2N(c2ccccc2)c2ccc3c(c2)C2(c4ccccc4-c4ccccc42)c2ccccc2-3)cc1]

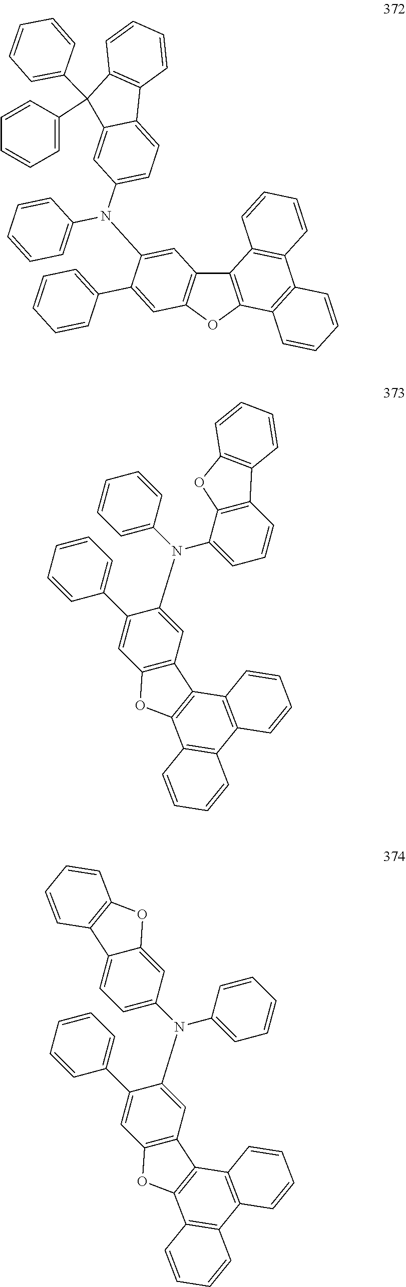 [SMILES: c1ccc(-c2cc3oc4c5ccccc5c5ccccc5c4c3cc2N(c2ccccc2)c2ccc3c(c2)C(c2ccccc2)(c2ccccc2)c2ccccc2-3)cc1 ; c1ccc(-c2cc3oc4c5ccccc5c5ccccc5c4c3cc2N(c2ccccc2)c2cccc3c2oc2ccccc23)cc1 ; c1ccc(-c2cc3oc4c5ccccc5c5ccccc5c4c3cc2N(c2ccccc2)c2ccc3c(c2)oc2ccccc23)cc1]

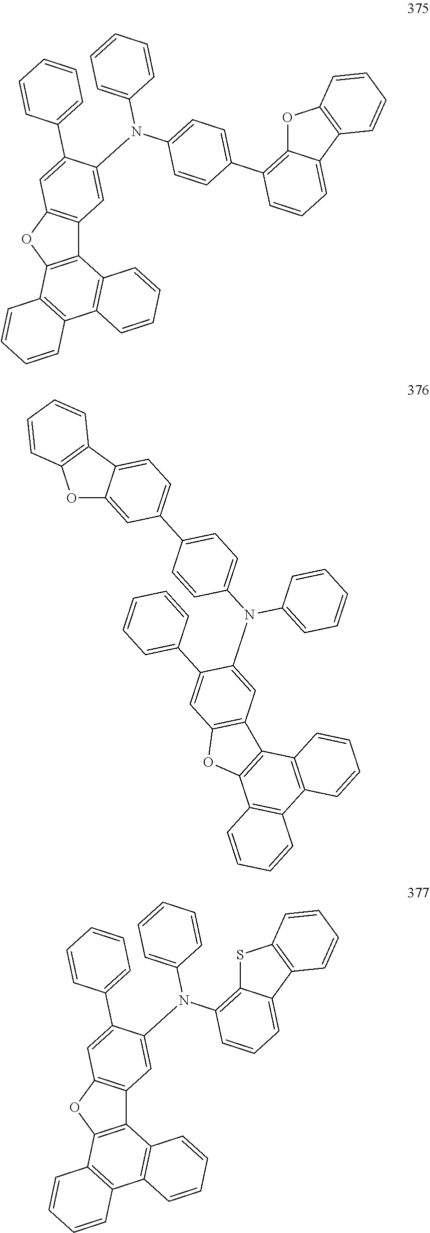

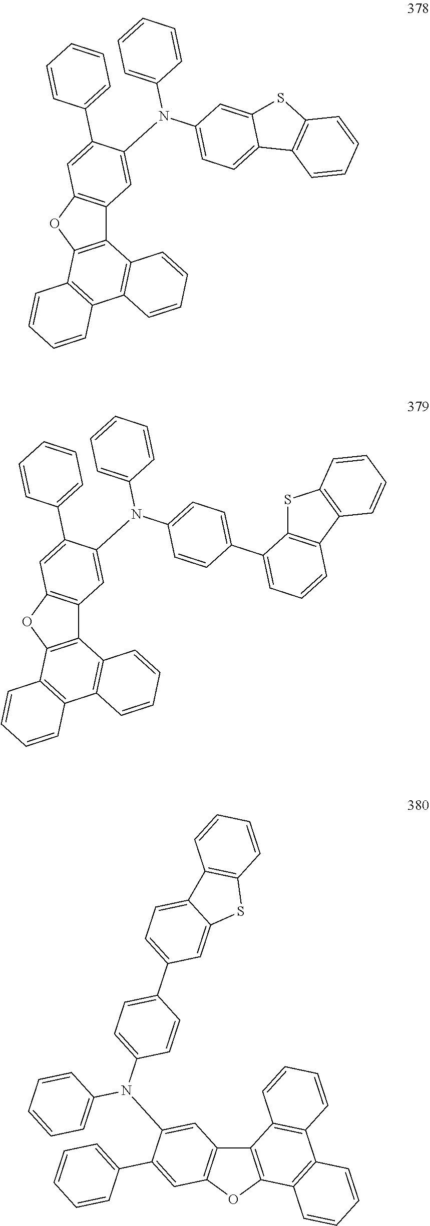 [SMILES: c1ccc(-c2cc3oc4c5ccccc5c5ccccc5c4c3cc2N(c2ccccc2)c2ccc3c(c2)sc2ccccc23)cc1 ; c1ccc(-c2cc3oc4c5ccccc5c5ccccc5c4c3cc2N(c2ccccc2)c2ccc(-c3cccc4c3sc3ccccc34)cc2)cc1 ; c1ccc(-c2cc3oc4c5ccccc5c5ccccc5c4c3cc2N(c2ccccc2)c2ccc(-c3ccc4c(c3)sc3ccccc34)cc2)cc1]

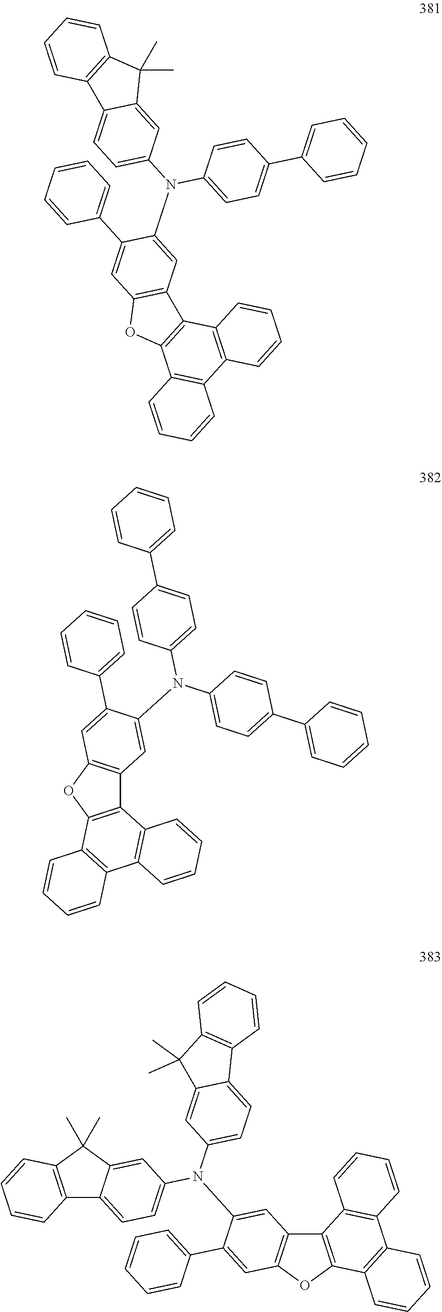 [SMILES: CC1(C)c2ccccc2-c2ccc(N(c3ccc(-c4ccccc4)cc3)c3cc4c(cc3-c3ccccc3)oc3c5ccccc5c5ccccc5c43)cc21 ; c1ccc(-c2ccc(N(c3ccc(-c4ccccc4)cc3)c3cc4c(cc3-c3ccccc3)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccccc3)oc3c5ccccc5c5ccccc5c43)cc21]

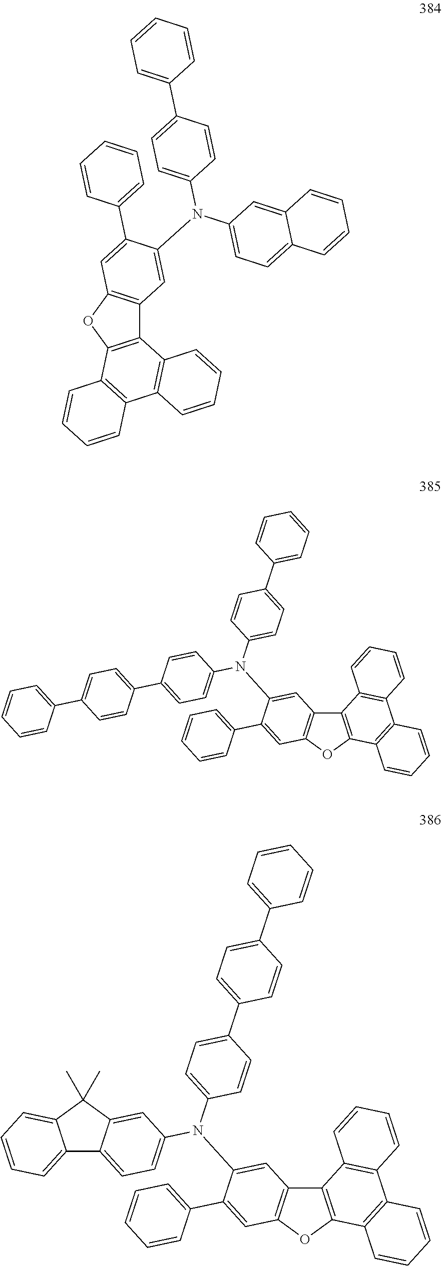 [SMILES: c1ccc(-c2ccc(N(c3ccc4ccccc4c3)c3cc4c(cc3-c3ccccc3)oc3c5ccccc5c5ccccc5c43)cc2)cc1 ; c1ccc(-c2ccc(-c3ccc(N(c4ccc(-c5ccccc5)cc4)c4cc5c(cc4-c4ccccc4)oc4c6ccccc6c6ccccc6c54)cc3)cc2)cc1 ; CC1(C)c2ccccc2-c2ccc(N(c3ccc(-c4ccc(-c5ccccc5)cc4)cc3)c3cc4c(cc3-c3ccccc3)oc3c5ccccc5c5ccccc5c43)cc21]

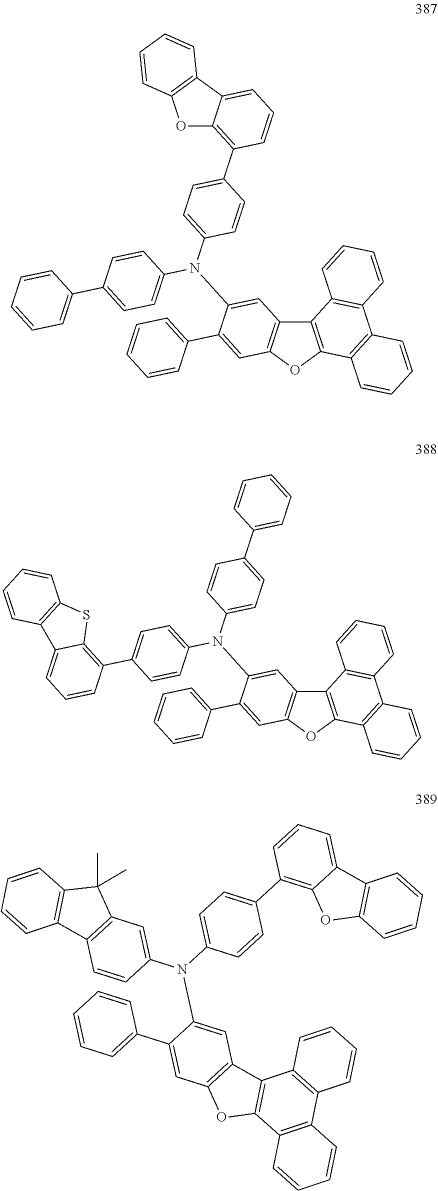 [SMILES: c1ccc(-c2ccc(N(c3ccc(-c4cccc5c4oc4ccccc45)cc3)c3cc4c(cc3-c3ccccc3)oc3c5ccccc5c5ccccc5c43)cc2)cc1 ; c1ccc(-c2ccc(N(c3ccc(-c4cccc5c4sc4ccccc45)cc3)c3cc4c(cc3-c3ccccc3)oc3c5ccccc5c5ccccc5c43)cc2)cc1 ; CC1(C)c2ccccc2-c2ccc(N(c3ccc(-c4cccc5c4oc4ccccc45)cc3)c3cc4c(cc3-c3ccccc3)oc3c5ccccc5c5ccccc5c43)cc21]

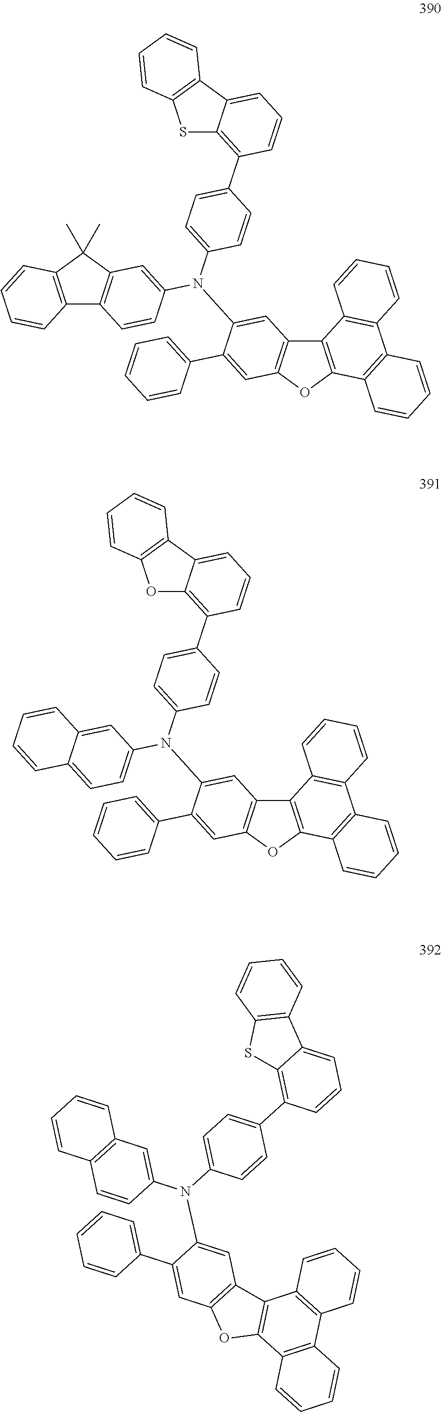 [SMILES: CC1(C)c2ccccc2-c2ccc(N(c3ccc(-c4cccc5c4sc4ccccc45)cc3)c3cc4c(cc3-c3ccccc3)oc3c5ccccc5c5ccccc5c43)cc21 ; c1ccc(-c2cc3oc4c5ccccc5c5ccccc5c4c3cc2N(c2ccc(-c3cccc4c3oc3ccccc34)cc2)c2ccc3ccccc3c2)cc1 ; c1ccc(-c2cc3oc4c5ccccc5c5ccccc5c4c3cc2N(c2ccc(-c3cccc4c3sc3ccccc34)cc2)c2ccc3ccccc3c2)cc1]

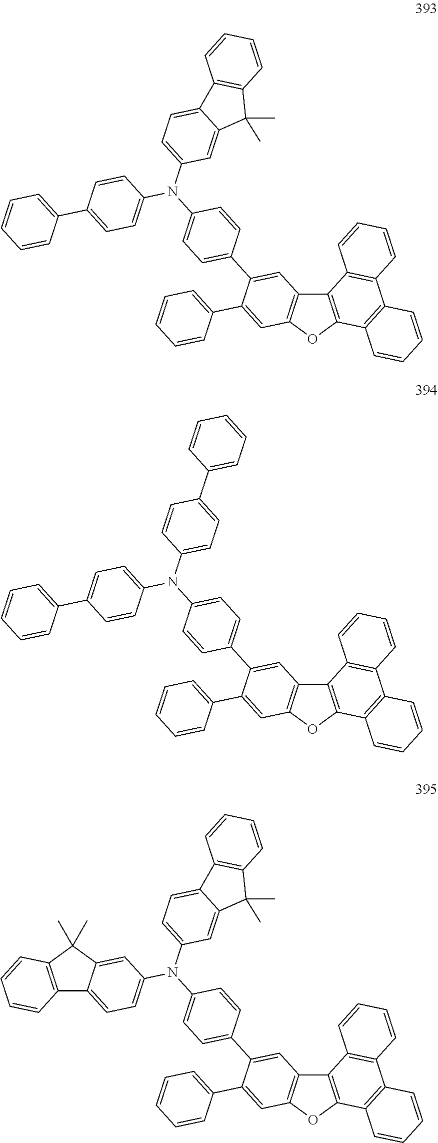 [SMILES: CC1(C)c2ccccc2-c2ccc(N(c3ccc(-c4ccccc4)cc3)c3ccc(-c4cc5c(cc4-c4ccccc4)oc4c6ccccc6c6ccccc6c54)cc3)cc21 ; c1ccc(-c2ccc(N(c3ccc(-c4ccccc4)cc3)c3ccc(-c4cc5c(cc4-c4ccccc4)oc4c6ccccc6c6ccccc6c54)cc3)cc2)cc1 ; CC1(C)c2ccccc2-c2ccc(N(c3ccc(-c4cc5c(cc4-c4ccccc4)oc4c6ccccc6c6ccccc6c54)cc3)c3ccc4c(c3)C(C)(C)c3ccccc3-4)cc21]

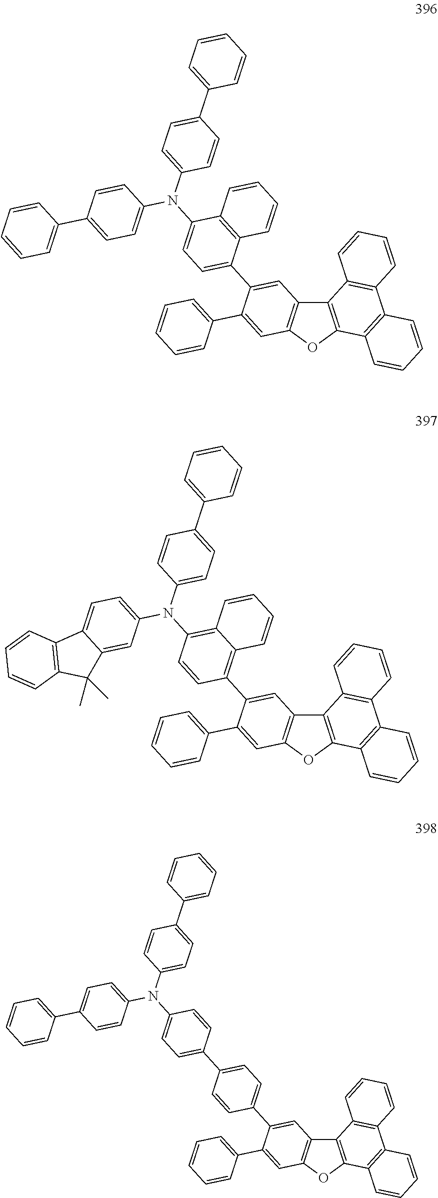 [SMILES: c1ccc(-c2ccc(N(c3ccc(-c4ccccc4)cc3)c3ccc(-c4cc5c(cc4-c4ccccc4)oc4c6ccccc6c6ccccc6c54)c4ccccc34)cc2)cc1 ; CC1(C)c2ccccc2-c2ccc(N(c3ccc(-c4ccccc4)cc3)c3ccc(-c4cc5c(cc4-c4ccccc4)oc4c6ccccc6c6ccccc6c54)c4ccccc34)cc21 ; c1ccc(-c2ccc(N(c3ccc(-c4ccccc4)cc3)c3ccc(-c4ccc(-c5cc6c(cc5-c5ccccc5)oc5c7ccccc7c7ccccc7c65)cc4)cc3)cc2)cc1]

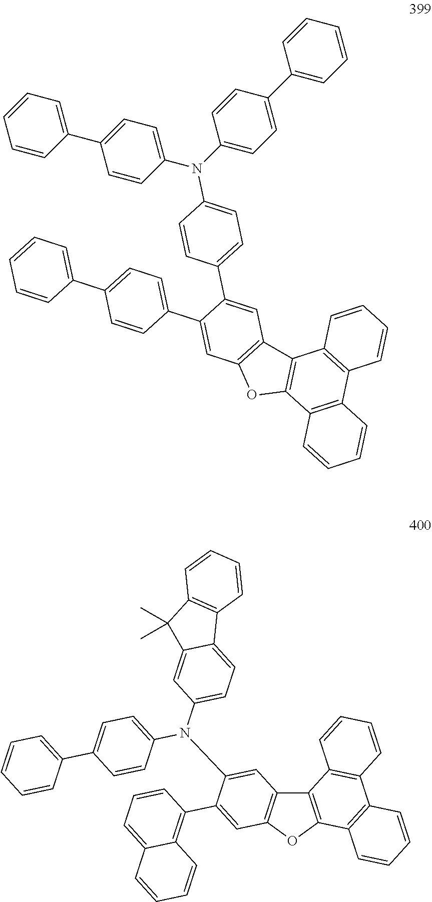 [SMILES: c1ccc(-c2ccc(-c3cc4oc5c6ccccc6c6ccccc6c5c4cc3-c3ccc(N(c4ccc(-c5ccccc5)cc4)c4ccc(-c5ccccc5)cc4)cc3)cc2)cc1 ; CC1(C)c2ccccc2-c2ccc(N(c3ccc(-c4ccccc4)cc3)c3cc4c(cc3-c3cccc5ccccc35)oc3c5ccccc5c5ccccc5c43)cc21]

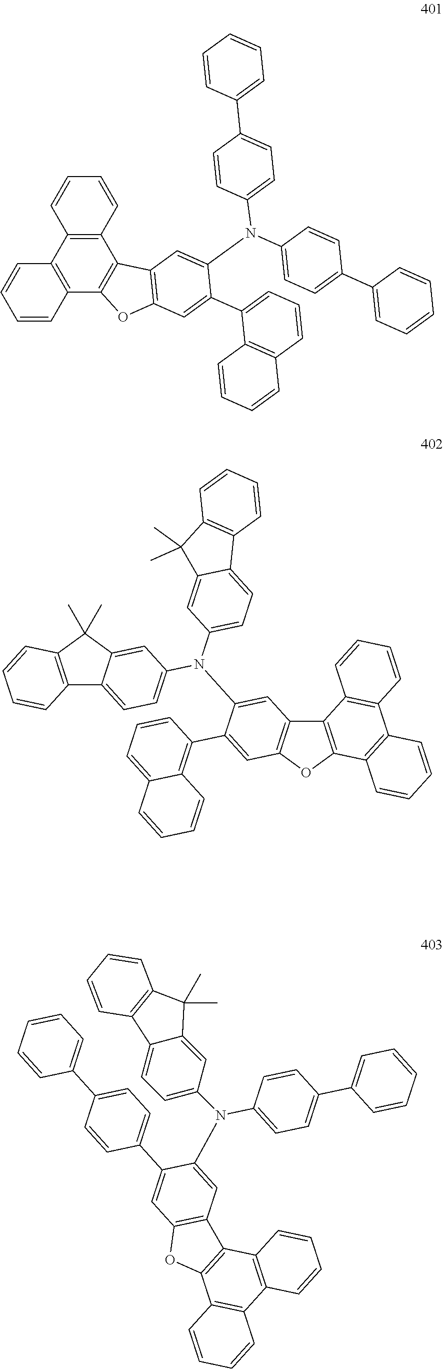 [SMILES: c1ccc(-c2ccc(N(c3ccc(-c4ccccc4)cc3)c3cc4c(cc3-c3cccc5ccccc35)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3cccc5ccccc35)oc3c5ccccc5c5ccccc5c43)cc21 ; CC1(C)c2ccccc2-c2ccc(N(c3ccc(-c4ccccc4)cc3)c3cc4c(cc3-c3ccc(-c5ccccc5)cc3)oc3c5ccccc5c5ccccc5c43)cc21]

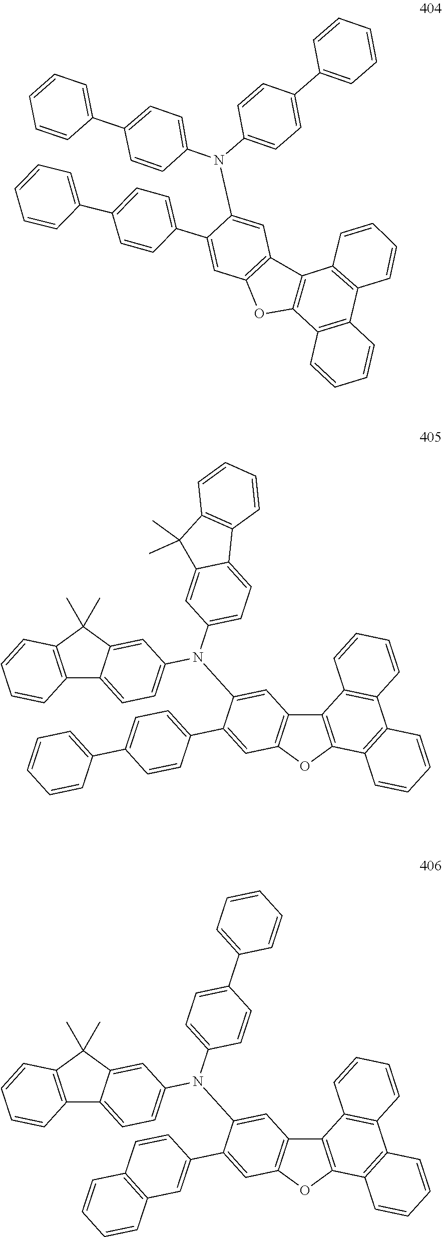 [SMILES: c1ccc(-c2ccc(-c3cc4oc5c6ccccc6c6ccccc6c5c4cc3N(c3ccc(-c4ccccc4)cc3)c3ccc(-c4ccccc4)cc3)cc2)cc1 ; CC1(C)c2ccccc2-c2ccc(N(c3ccc4c(c3)C(C)(C)c3ccccc3-4)c3cc4c(cc3-c3ccc(-c5ccccc5)cc3)oc3c5ccccc5c5ccccc5c43)cc21 ; CC1(C)c2ccccc2-c2ccc(N(c3ccc(-c4ccccc4)cc3)c3cc4c(cc3-c3ccc5ccccc5c3)oc3c5ccccc5c5ccccc5c43)cc21]

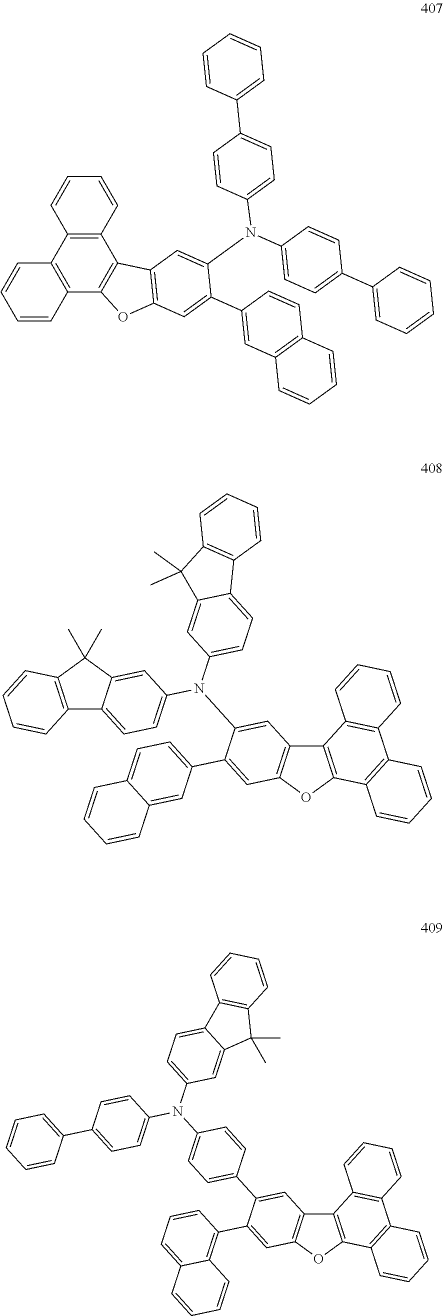 [SMILES: c1ccc(-c2ccc(N(c3ccc(-c4ccccc4)cc3)c3cc4c(cc3-c3ccc5ccccc5c3)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccc5ccccc5c3)oc3c5ccccc5c5ccccc5c43)cc21 ; CC1(C)c2ccccc2-c2ccc(N(c3ccc(-c4ccccc4)cc3)c3ccc(-c4cc5c(cc4-c4cccc6ccccc46)oc4c6ccccc6c6ccccc6c54)cc3)cc21]

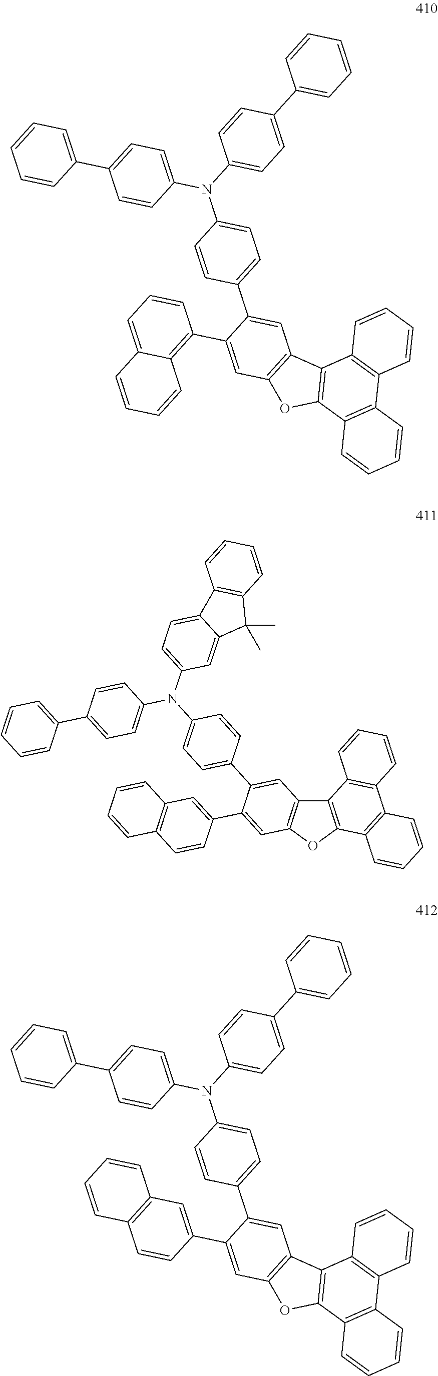 [SMILES: c1ccc(-c2ccc(N(c3ccc(-c4ccccc4)cc3)c3ccc(-c4cc5c(cc4-c4cccc6ccccc46)oc4c6ccccc6c6ccccc6c54)cc3)cc2)cc1 ; CC1(C)c2ccccc2-c2ccc(N(c3ccc(-c4ccccc4)cc3)c3ccc(-c4cc5c(cc4-c4ccc6ccccc6c4)oc4c6ccccc6c6ccccc6c54)cc3)cc21 ; c1ccc(-c2ccc(N(c3ccc(-c4ccccc4)cc3)c3ccc(-c4cc5c(cc4-c4ccc6ccccc6c4)oc4c6ccccc6c6ccccc6c54)cc3)cc2)cc1]

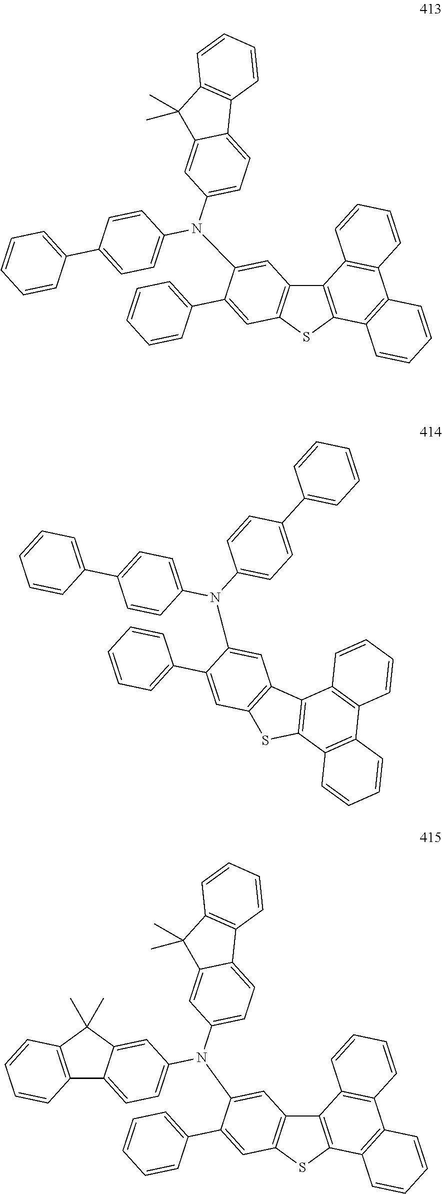 [SMILES: CC1(C)c2ccccc2-c2ccc(N(c3ccc(-c4ccccc4)cc3)c3cc4c(cc3-c3ccccc3)sc3c5ccccc5c5ccccc5c43)cc21 ; c1ccc(-c2ccc(N(c3ccc(-c4ccccc4)cc3)c3cc4c(cc3-c3ccccc3)sc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccccc3)sc3c5ccccc5c5ccccc5c43)cc21]

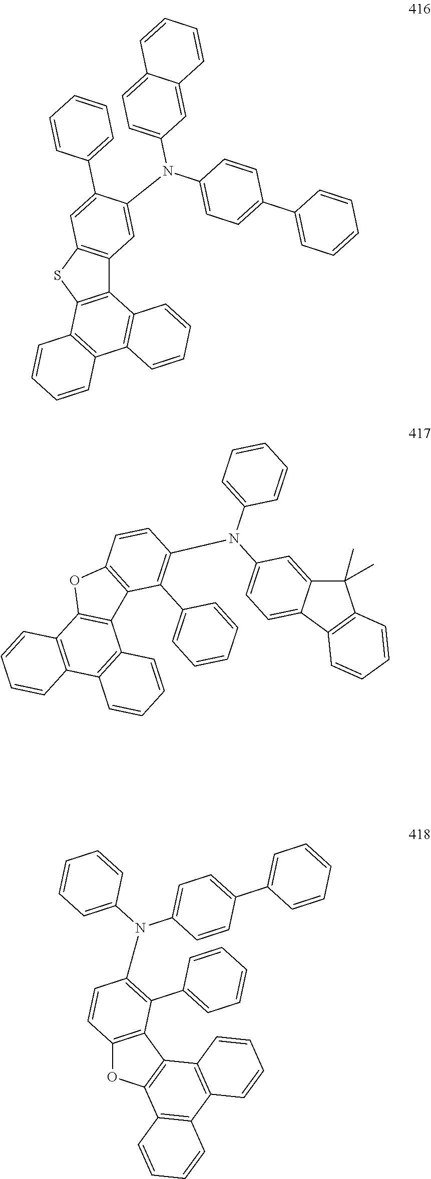 [SMILES: c1ccc(-c2ccc(N(c3ccc4ccccc4c3)c3cc4c(cc3-c3ccccc3)sc3c5ccccc5c5ccccc5c43)cc2)cc1 ; CC1(C)c2ccccc2-c2ccc(N(c3ccccc3)c3ccc4oc5c6ccccc6c6ccccc6c5c4c3-c3ccccc3)cc21 ; c1ccc(-c2ccc(N(c3ccccc3)c3ccc4oc5c6ccccc6c6ccccc6c5c4c3-c3ccccc3)cc2)cc1]

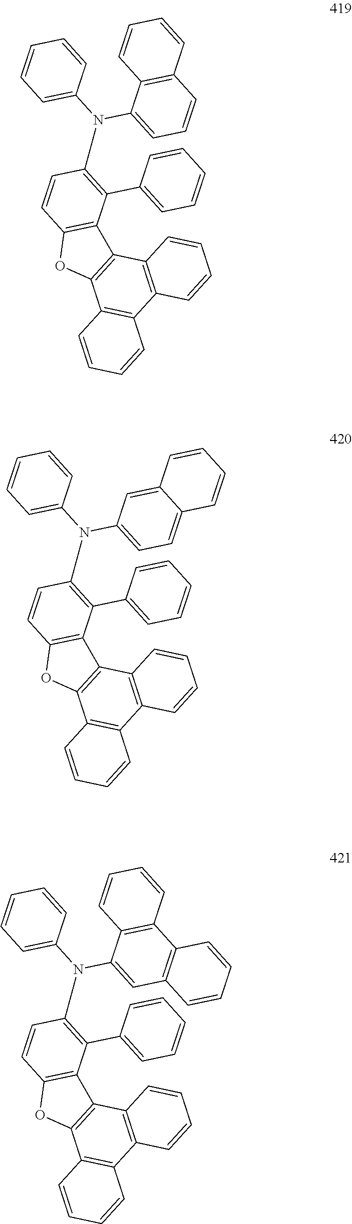 [SMILES: c1ccc(-c2c(N(c3ccccc3)c3cccc4ccccc34)ccc3oc4c5ccccc5c5ccccc5c4c23)cc1 ; c1ccc(-c2c(N(c3ccccc3)c3ccc4ccccc4c3)ccc3oc4c5ccccc5c5ccccc5c4c23)cc1 ; c1ccc(-c2c(N(c3ccccc3)c3cc4ccccc4c4ccccc34)ccc3oc4c5ccccc5c5ccccc5c4c23)cc1]

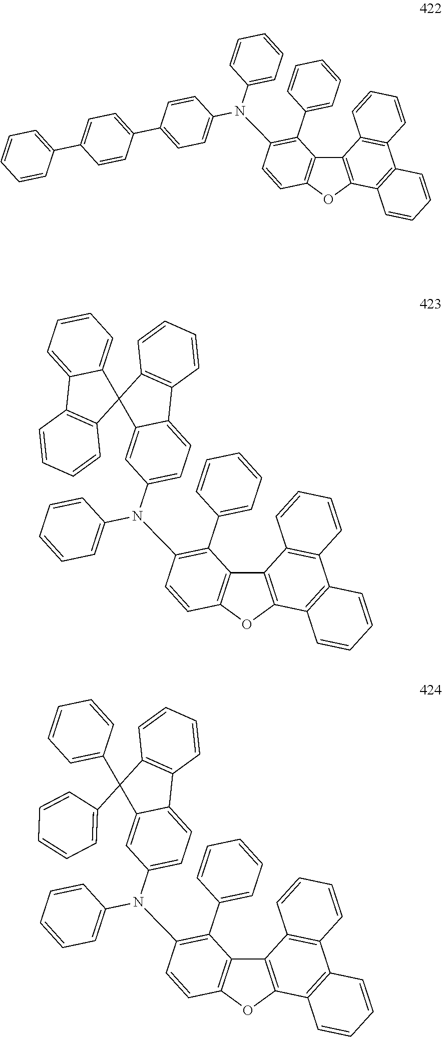 [SMILES: c1ccc(-c2ccc(-c3ccc(N(c4ccccc4)c4ccc5oc6c7ccccc7c7ccccc7c6c5c4-c4ccccc4)cc3)cc2)cc1 ; c1ccc(-c2c(N(c3ccccc3)c3ccc4c(c3)C3(c5ccccc5-c5ccccc53)c3ccccc3-4)ccc3oc4c5ccccc5c5ccccc5c4c23)cc1 ; c1ccc(-c2c(N(c3ccccc3)c3ccc4c(c3)C(c3ccccc3)(c3ccccc3)c3ccccc3-4)ccc3oc4c5ccccc5c5ccccc5c4c23)cc1]

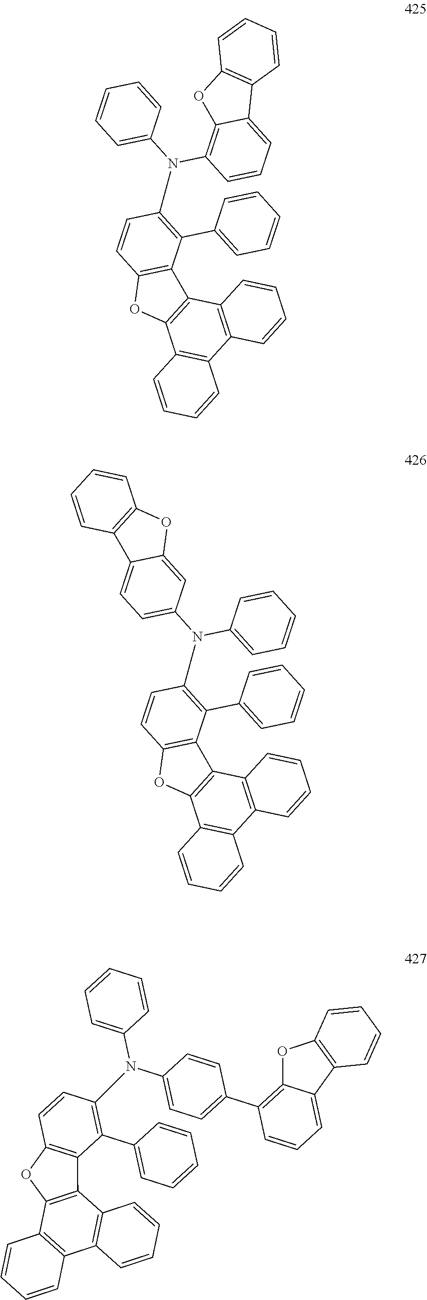 [SMILES: c1ccc(-c2c(N(c3ccccc3)c3cccc4c3oc3ccccc34)ccc3oc4c5ccccc5c5ccccc5c4c23)cc1 ; c1ccc(-c2c(N(c3ccccc3)c3ccc4c(c3)oc3ccccc34)ccc3oc4c5ccccc5c5ccccc5c4c23)cc1 ; c1ccc(-c2c(N(c3ccccc3)c3ccc(-c4cccc5c4oc4ccccc45)cc3)ccc3oc4c5ccccc5c5ccccc5c4c23)cc1]

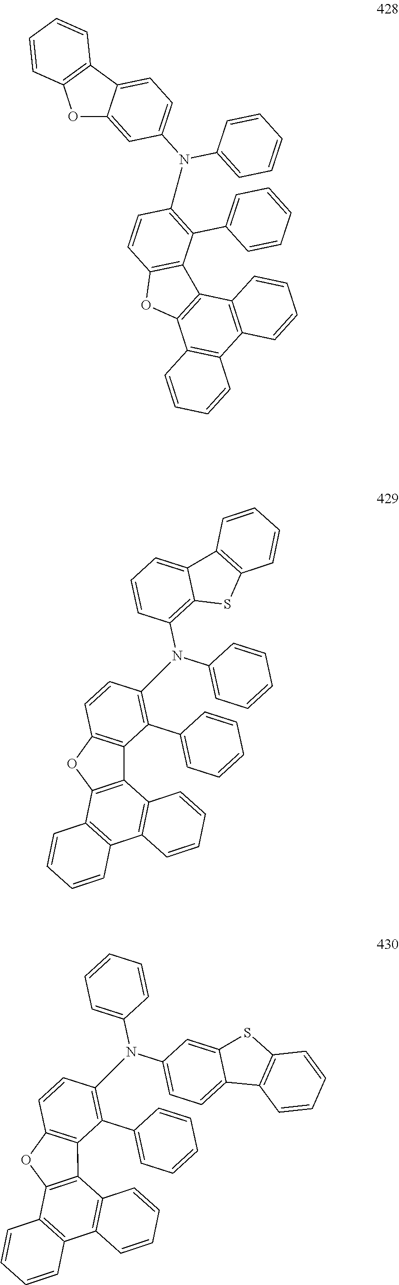 [SMILES: c1ccc(-c2c(N(c3ccccc3)c3ccc4c(c3)oc3ccccc34)ccc3oc4c5ccccc5c5ccccc5c4c23)cc1 ; c1ccc(-c2c(N(c3ccccc3)c3cccc4c3sc3ccccc34)ccc3oc4c5ccccc5c5ccccc5c4c23)cc1 ; c1ccc(-c2c(N(c3ccccc3)c3ccc4c(c3)sc3ccccc34)ccc3oc4c5ccccc5c5ccccc5c4c23)cc1]

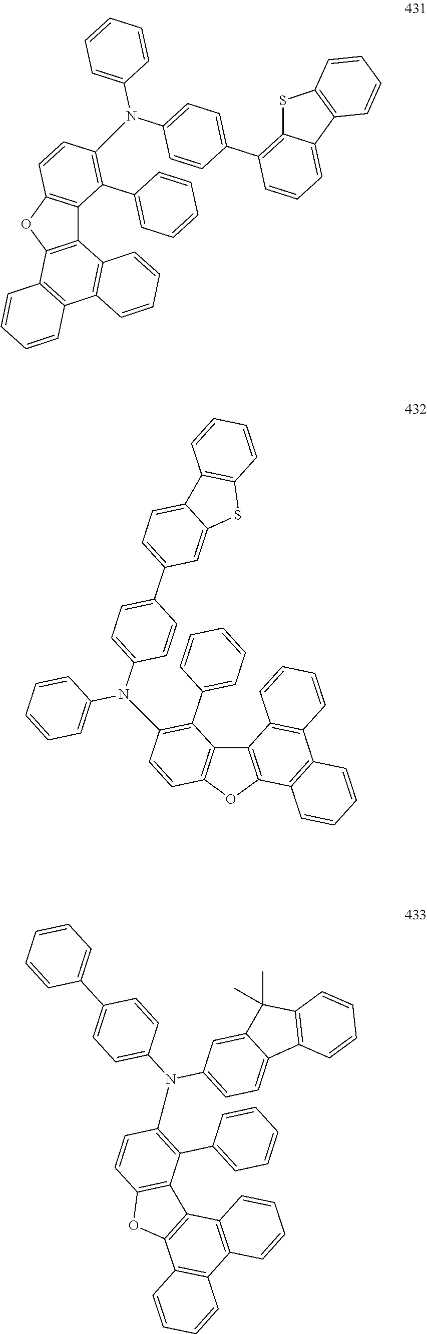 [SMILES: c1ccc(-c2c(N(c3ccccc3)c3ccc(-c4cccc5c4sc4ccccc45)cc3)ccc3oc4c5ccccc5c5ccccc5c4c23)cc1 ; c1ccc(-c2c(N(c3ccccc3)c3ccc(-c4ccc5c(c4)sc4ccccc45)cc3)ccc3oc4c5ccccc5c5ccccc5c4c23)cc1 ; CC1(C)c2ccccc2-c2ccc(N(c3ccc(-c4ccccc4)cc3)c3ccc4oc5c6ccccc6c6ccccc6c5c4c3-c3ccccc3)cc21]

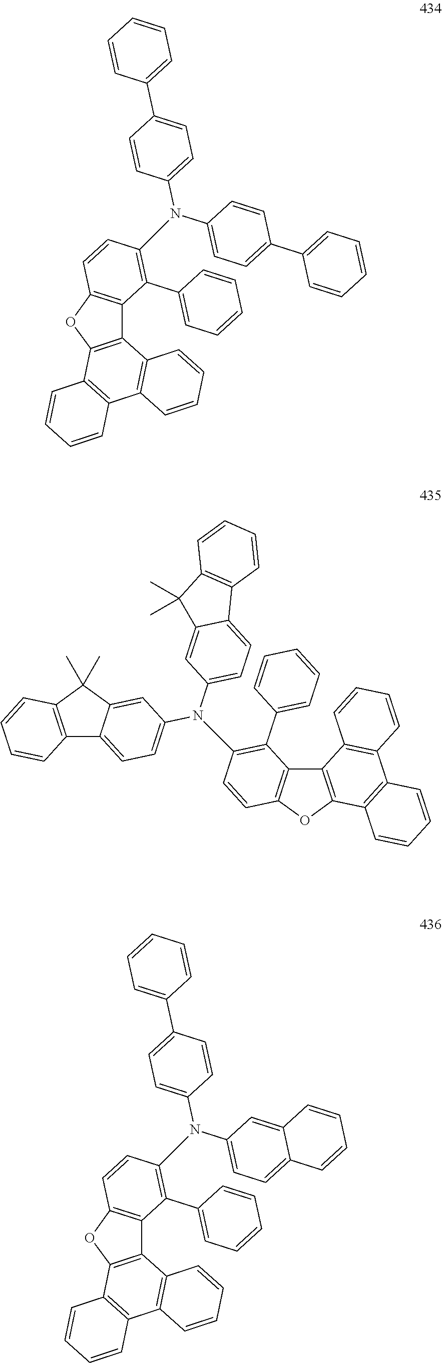 [SMILES: c1ccc(-c2ccc(N(c3ccc(-c4ccccc4)cc3)c3ccc4oc5c6ccccc6c6ccccc6c5c4c3-c3ccccc3)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccccc3)cc21 ; c1ccc(-c2ccc(N(c3ccc4ccccc4c3)c3ccc4oc5c6ccccc6c6ccccc6c5c4c3-c3ccccc3)cc2)cc1]

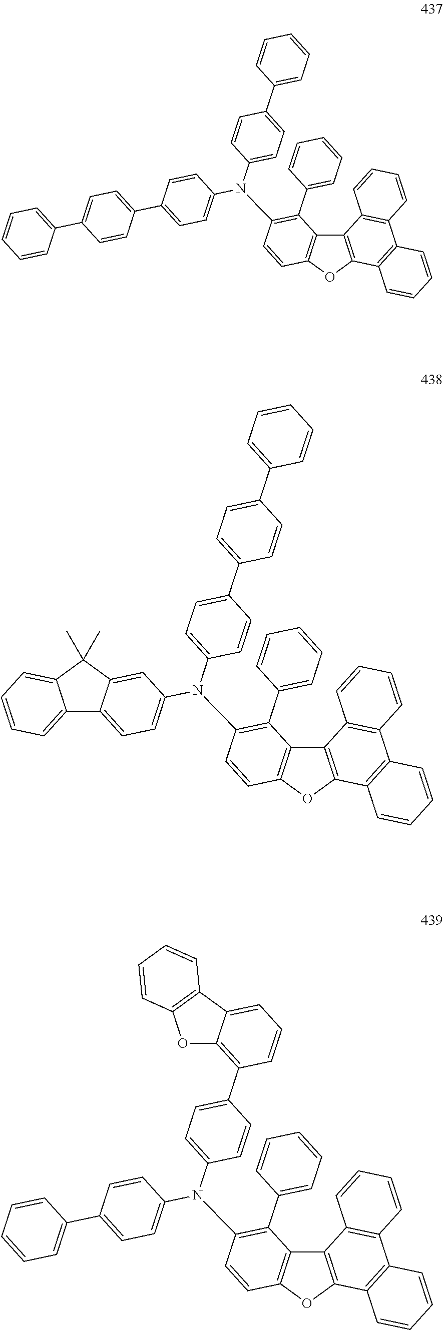 [SMILES: c1ccc(-c2ccc(-c3ccc(N(c4ccc(-c5ccccc5)cc4)c4ccc5oc6c7ccccc7c7ccccc7c6c5c4-c4ccccc4)cc3)cc2)cc1 ; CC1(C)c2ccccc2-c2ccc(N(c3ccc(-c4ccc(-c5ccccc5)cc4)cc3)c3ccc4oc5c6ccccc6c6ccccc6c5c4c3-c3ccccc3)cc21 ; c1ccc(-c2ccc(N(c3ccc(-c4cccc5c4oc4ccccc45)cc3)c3ccc4oc5c6ccccc6c6ccccc6c5c4c3-c3ccccc3)cc2)cc1]

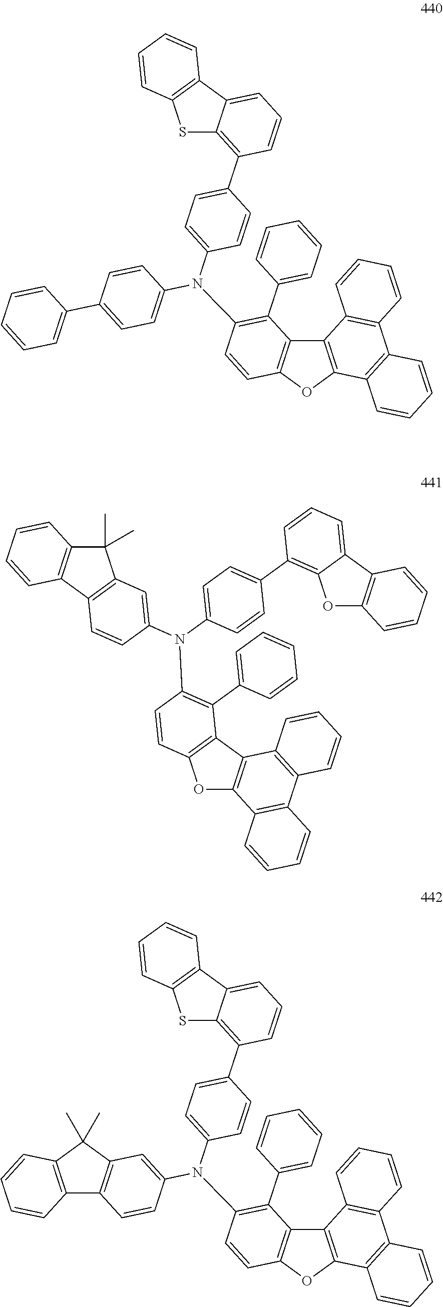 [SMILES: c1ccc(-c2ccc(N(c3ccc(-c4cccc5c4sc4ccccc45)cc3)c3ccc4oc5c6ccccc6c6ccccc6c5c4c3-c3ccccc3)cc2)cc1 ; CC1(C)c2ccccc2-c2ccc(N(c3ccc(-c4cccc5c4oc4ccccc45)cc3)c3ccc4oc5c6ccccc6c6ccccc6c5c4c3-c3ccccc3)cc21 ; CC1(C)c2ccccc2-c2ccc(N(c3ccc(-c4cccc5c4sc4ccccc45)cc3)c3ccc4oc5c6ccccc6c6ccccc6c5c4c3-c3ccccc3)cc21]

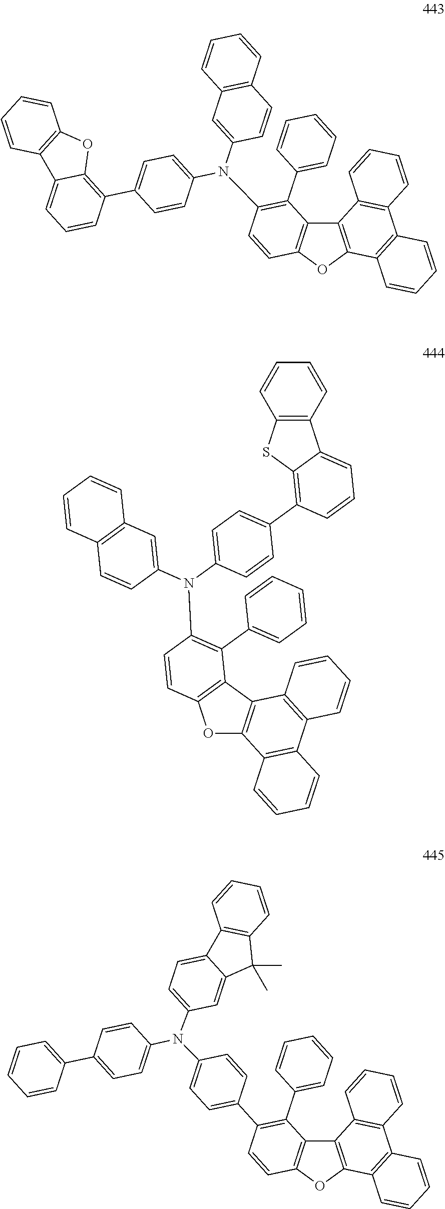 [SMILES: c1ccc(-c2c(N(c3ccc(-c4cccc5c4oc4ccccc45)cc3)c3ccc4ccccc4c3)ccc3oc4c5ccccc5c5ccccc5c4c23)cc1 ; c1ccc(-c2c(N(c3ccc(-c4cccc5c4sc4ccccc45)cc3)c3ccc4ccccc4c3)ccc3oc4c5ccccc5c5ccccc5c4c23)cc1 ; CC1(C)c2ccccc2-c2ccc(N(c3ccc(-c4ccccc4)cc3)c3ccc(-c4ccc5oc6c7ccccc7c7ccccc7c6c5c4-c4ccccc4)cc3)cc21]

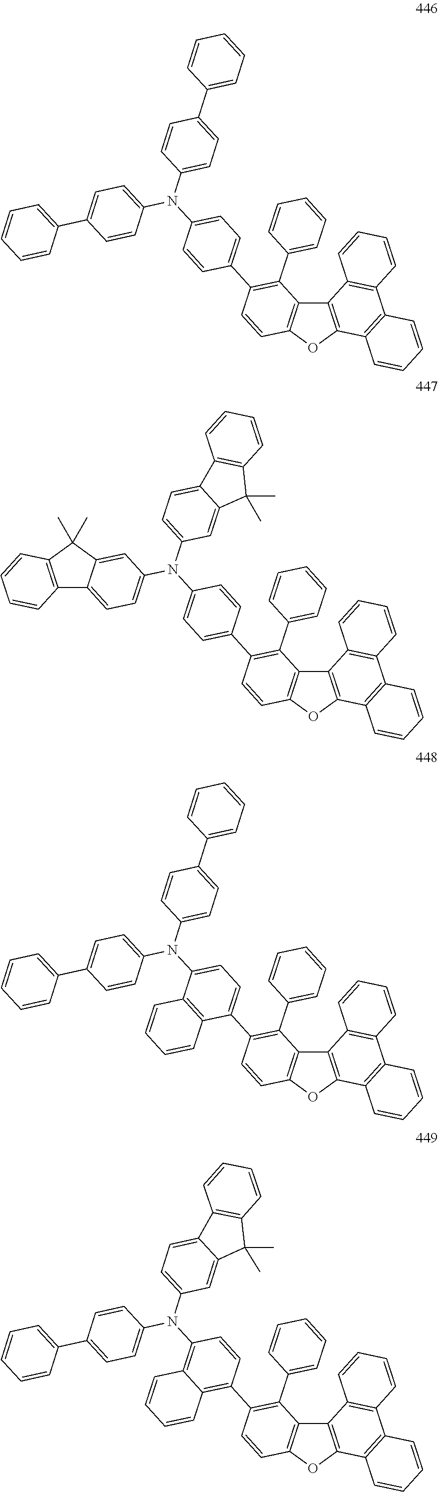 [SMILES: c1ccc(-c2ccc(N(c3ccc(-c4ccccc4)cc3)c3ccc(-c4ccc5oc6c7ccccc7c7ccccc7c6c5c4-c4ccccc4)cc3)cc2)cc1 ; CC1(C)c2ccccc2-c2ccc(N(c3ccc(-c4ccc5oc6c7ccccc7c7ccccc7c6c5c4-c4ccccc4)cc3)c3ccc4c(c3)C(C)(C)c3ccccc3-4)cc21 ; c1ccc(-c2ccc(N(c3ccc(-c4ccccc4)cc3)c3ccc(-c4ccc5oc6c7ccccc7c7ccccc7c6c5c4-c4ccccc4)c4ccccc34)cc2)cc1 ; CC1(C)c2ccccc2-c2ccc(N(c3ccc(-c4ccccc4)cc3)c3ccc(-c4ccc5oc6c7ccccc7c7ccccc7c6c5c4-c4ccccc4)c4ccccc34)cc21]

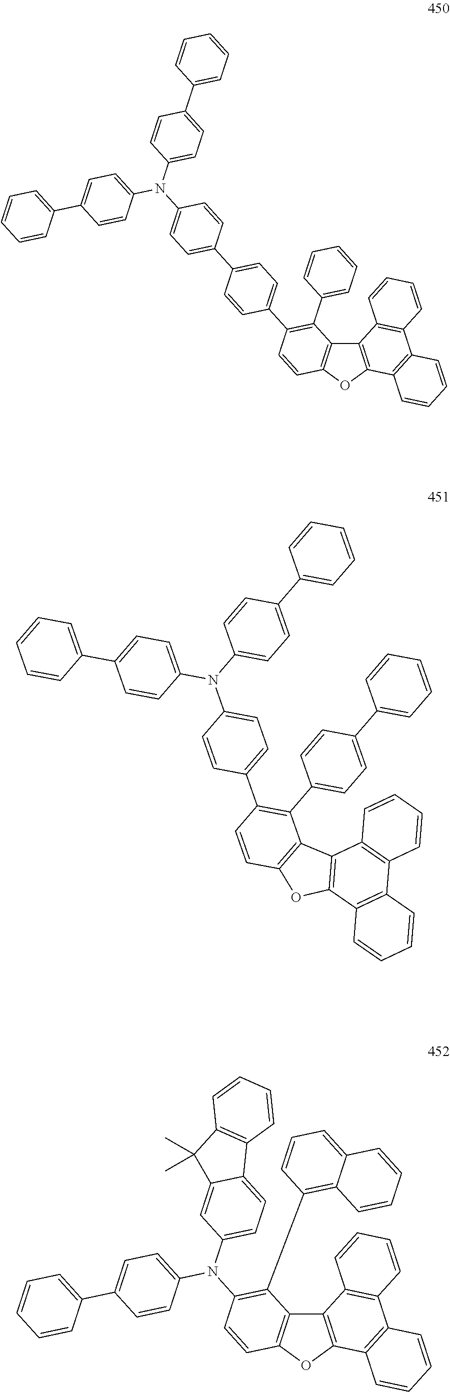 [SMILES: c1ccc(-c2ccc(N(c3ccc(-c4ccccc4)cc3)c3ccc(-c4ccc(-c5ccc6oc7c8ccccc8c8ccccc8c7c6c5-c5ccccc5)cc4)cc3)cc2)cc1 ; c1ccc(-c2ccc(-c3c(-c4ccc(N(c5ccc(-c6ccccc6)cc5)c5ccc(-c6ccccc6)cc5)cc4)ccc4oc5c6ccccc6c6ccccc6c5c34)cc2)cc1 ; CC1(C)c2ccccc2-c2ccc(N(c3ccc(-c4ccccc4)cc3)c3ccc4oc5c6ccccc6c6ccccc6c5c4c3-c3cccc4ccccc34)cc21]

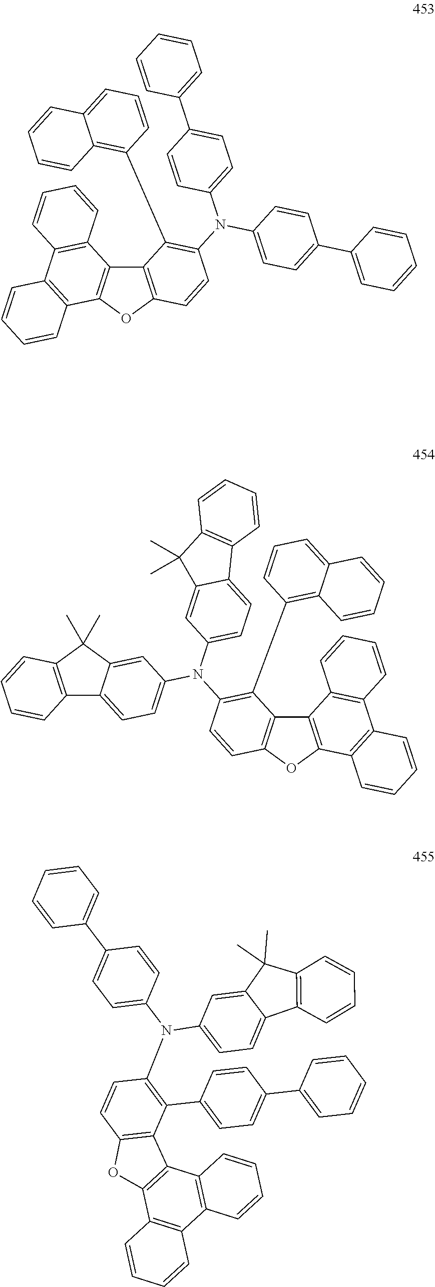 [SMILES: c1ccc(-c2ccc(N(c3ccc(-c4ccccc4)cc3)c3ccc4oc5c6ccccc6c6ccccc6c5c4c3-c3cccc4ccccc34)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3cccc4ccccc34)cc21 ; CC1(C)c2ccccc2-c2ccc(N(c3ccc(-c4ccccc4)cc3)c3ccc4oc5c6ccccc6c6ccccc6c5c4c3-c3ccc(-c4ccccc4)cc3)cc21]

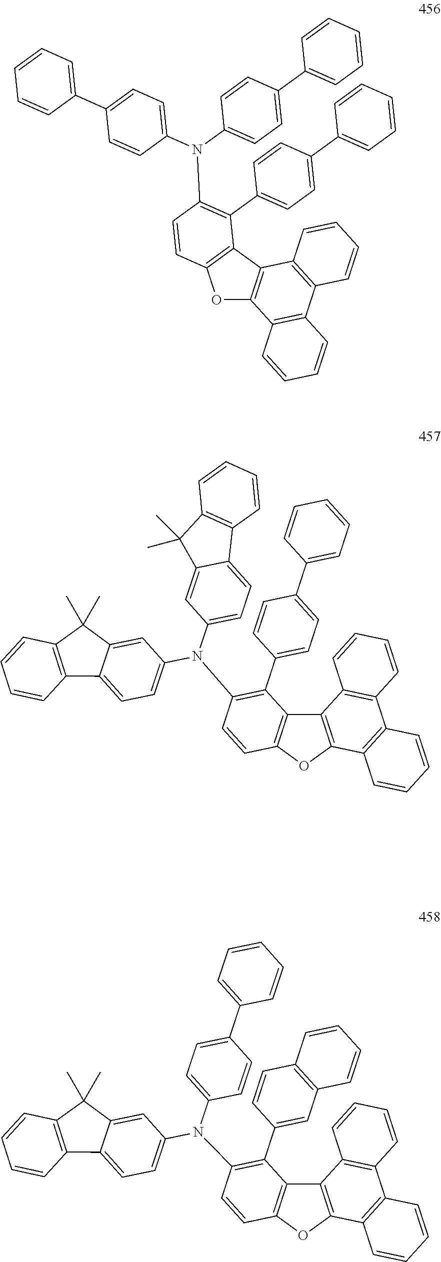 [SMILES: c1ccc(-c2ccc(-c3c(N(c4ccc(-c5ccccc5)cc4)c4ccc(-c5ccccc5)cc4)ccc4oc5c6ccccc6c6ccccc6c5c34)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccc(-c4ccccc4)cc3)cc21 ; CC1(C)c2ccccc2-c2ccc(N(c3ccc(-c4ccccc4)cc3)c3ccc4oc5c6ccccc6c6ccccc6c5c4c3-c3ccc4ccccc4c3)cc21]

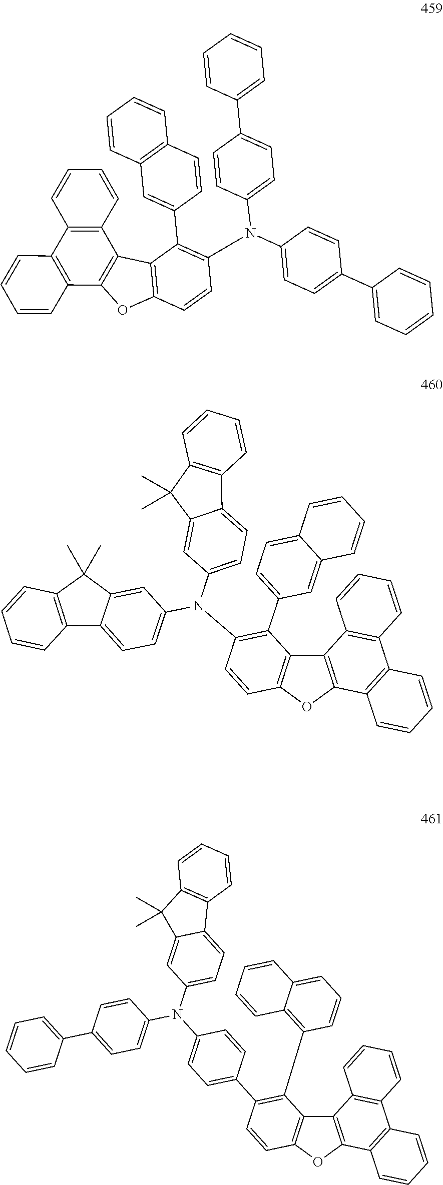 [SMILES: c1ccc(-c2ccc(N(c3ccc(-c4ccccc4)cc3)c3ccc4oc5c6ccccc6c6ccccc6c5c4c3-c3ccc4ccccc4c3)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccc4ccccc4c3)cc21 ; CC1(C)c2ccccc2-c2ccc(N(c3ccc(-c4ccccc4)cc3)c3ccc(-c4ccc5oc6c7ccccc7c7ccccc7c6c5c4-c4cccc5ccccc45)cc3)cc21]

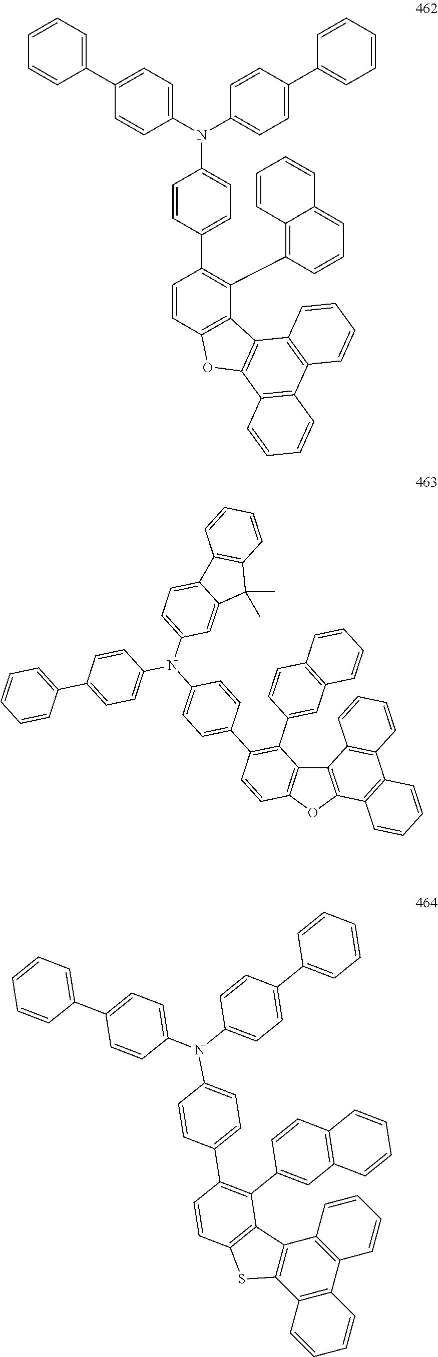 [SMILES: c1ccc(-c2ccc(N(c3ccc(-c4ccccc4)cc3)c3ccc(-c4ccc5oc6c7ccccc7c7ccccc7c6c5c4-c4cccc5ccccc45)cc3)cc2)cc1 ; CC1(C)c2ccccc2-c2ccc(N(c3ccc(-c4ccccc4)cc3)c3ccc(-c4ccc5oc6c7ccccc7c7ccccc7c6c5c4-c4ccc5ccccc5c4)cc3)cc21 ; c1ccc(-c2ccc(N(c3ccc(-c4ccccc4)cc3)c3ccc(-c4ccc5sc6c7ccccc7c7ccccc7c6c5c4-c4ccc5ccccc5c4)cc3)cc2)cc1]

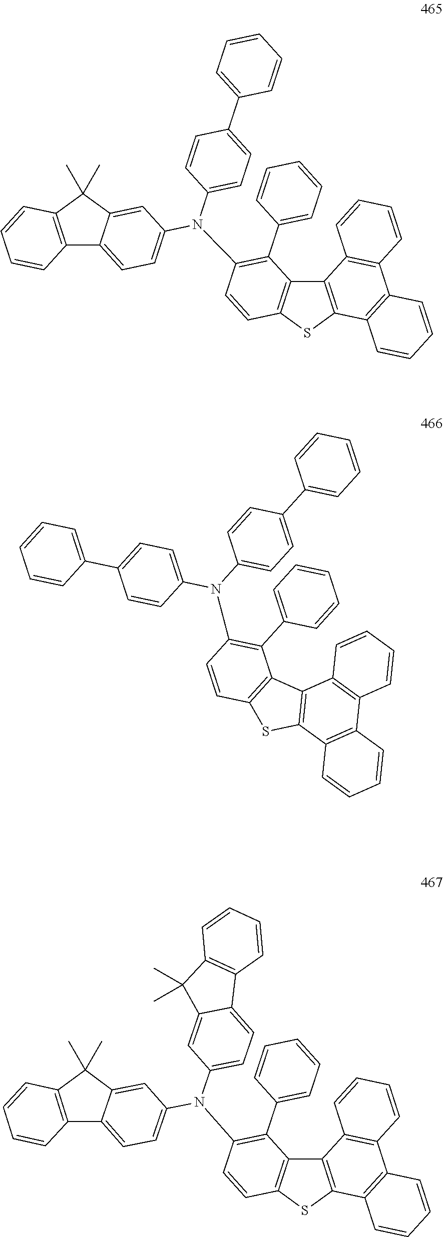 [SMILES: CC1(C)c2ccccc2-c2ccc(N(c3ccc(-c4ccccc4)cc3)c3ccc4sc5c6ccccc6c6ccccc6c5c4c3-c3ccccc3)cc21 ; c1ccc(-c2ccc(N(c3ccc(-c4ccccc4)cc3)c3ccc4sc5c6ccccc6c6ccccc6c5c4c3-c3ccccc3)cc2)cc1 ; CC1(C)c2ccccc2-c2ccc(N(c3ccc4c(c3)C(C)(C)c3ccccc3-4)c3ccc4sc5c6ccccc6c6ccccc6c5c4c3-c3ccccc3)cc21]

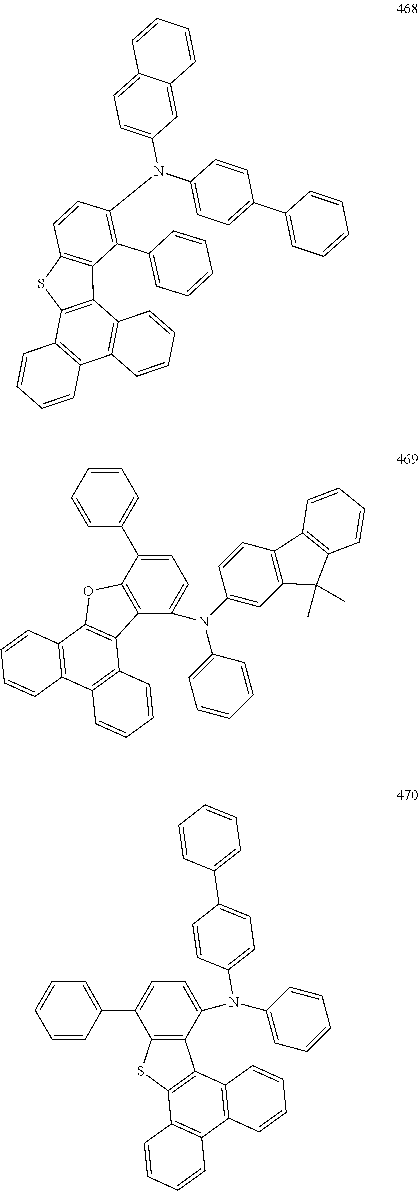 [SMILES: c1ccc(-c2ccc(N(c3ccc4ccccc4c3)c3ccc4sc5c6ccccc6c6ccccc6c5c4c3-c3ccccc3)cc2)cc1 ; CC1(C)c2ccccc2-c2ccc(N(c3ccccc3)c3ccc(-c4ccccc4)c4oc5c6ccccc6c6ccccc6c5c34)cc21 ; c1ccc(-c2ccc(N(c3ccccc3)c3ccc(-c4ccccc4)c4sc5c6ccccc6c6ccccc6c5c34)cc2)cc1]

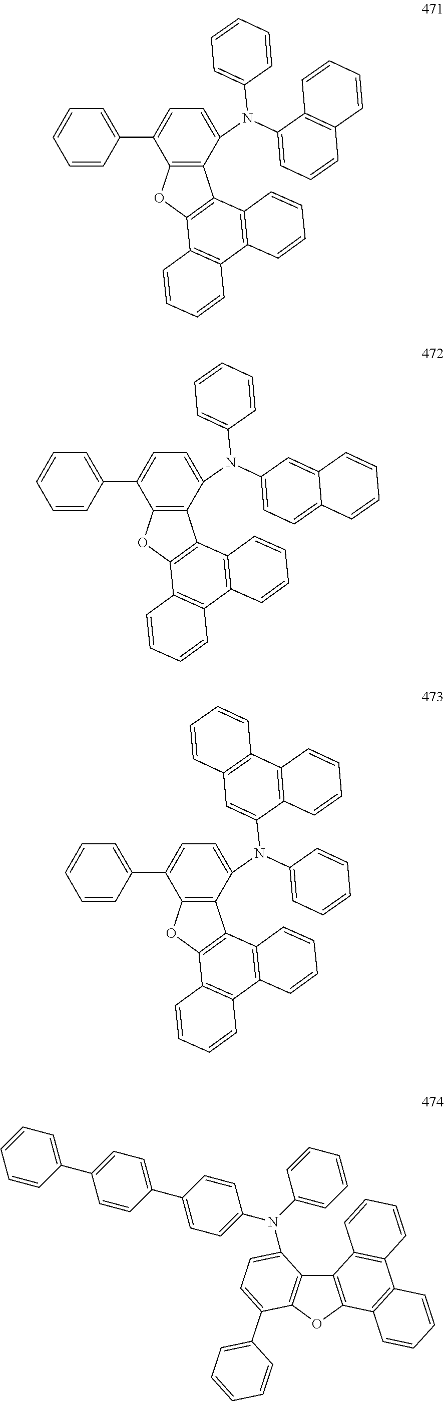 [SMILES: c1ccc(-c2ccc(N(c3ccccc3)c3cccc4ccccc34)c3c2oc2c4ccccc4c4ccccc4c23)cc1 ; c1ccc(-c2ccc(N(c3ccccc3)c3ccc4ccccc4c3)c3c2oc2c4ccccc4c4ccccc4c23)cc1 ; c1ccc(-c2ccc(N(c3ccccc3)c3cc4ccccc4c4ccccc34)c3c2oc2c4ccccc4c4ccccc4c23)cc1 ; c1ccc(-c2ccc(-c3ccc(N(c4ccccc4)c4ccc(-c5ccccc5)c5oc6c7ccccc7c7ccccc7c6c45)cc3)cc2)cc1]

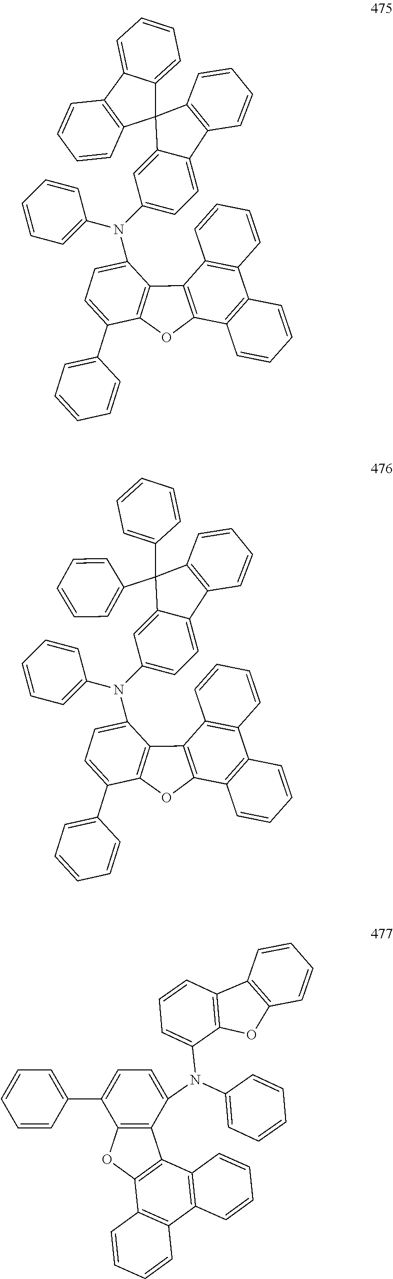 [SMILES: c1ccc(-c2ccc(N(c3ccccc3)c3ccc4c(c3)C3(c5ccccc5-c5ccccc53)c3ccccc3-4)c3c2oc2c4ccccc4c4ccccc4c23)cc1 ; c1ccc(-c2ccc(N(c3ccccc3)c3ccc4c(c3)C(c3ccccc3)(c3ccccc3)c3ccccc3-4)c3c2oc2c4ccccc4c4ccccc4c23)cc1 ; c1ccc(-c2ccc(N(c3ccccc3)c3cccc4c3oc3ccccc34)c3c2oc2c4ccccc4c4ccccc4c23)cc1]

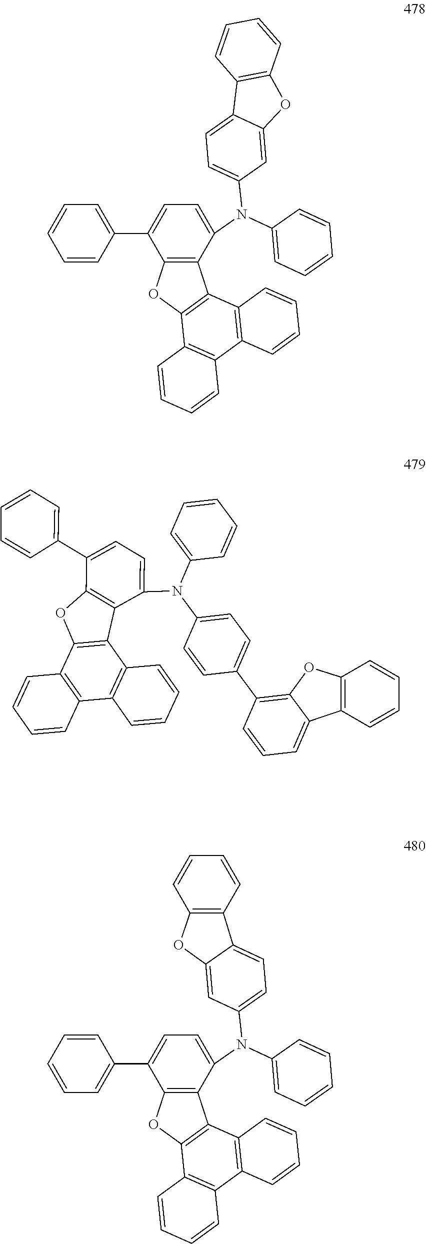 [SMILES: c1ccc(-c2ccc(N(c3ccccc3)c3ccc4c(c3)oc3ccccc34)c3c2oc2c4ccccc4c4ccccc4c23)cc1 ; c1ccc(-c2ccc(N(c3ccccc3)c3ccc(-c4cccc5c4oc4ccccc45)cc3)c3c2oc2c4ccccc4c4ccccc4c23)cc1 ; c1ccc(-c2ccc(N(c3ccccc3)c3ccc4c(c3)oc3ccccc34)c3c2oc2c4ccccc4c4ccccc4c23)cc1]

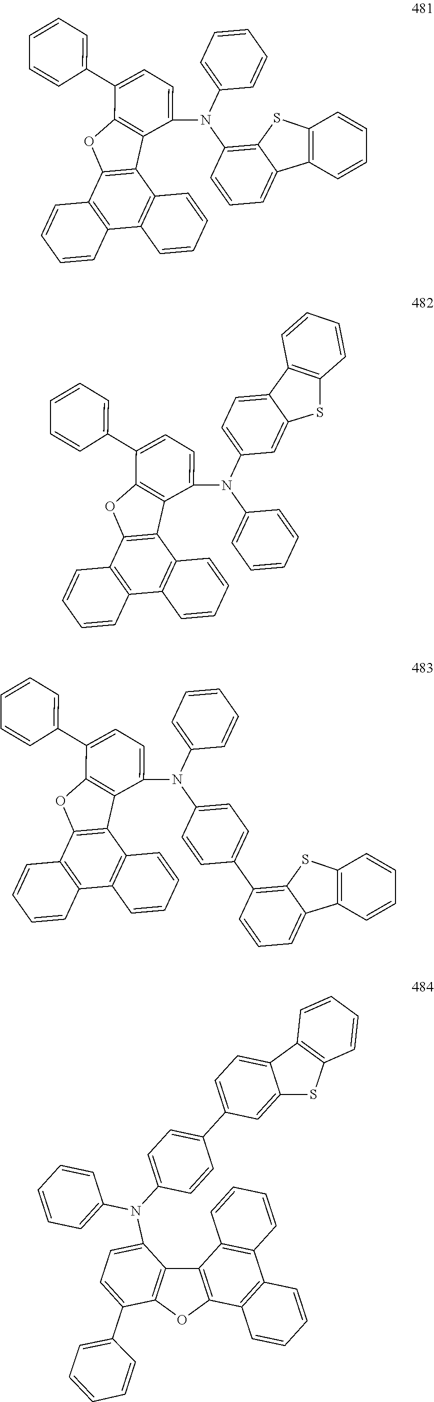 [SMILES: c1ccc(-c2ccc(N(c3ccccc3)c3cccc4c3sc3ccccc34)c3c2oc2c4ccccc4c4ccccc4c23)cc1 ; c1ccc(-c2ccc(N(c3ccccc3)c3ccc4c(c3)sc3ccccc34)c3c2oc2c4ccccc4c4ccccc4c23)cc1 ; c1ccc(-c2ccc(N(c3ccccc3)c3ccc(-c4cccc5c4sc4ccccc45)cc3)c3c2oc2c4ccccc4c4ccccc4c23)cc1 ; c1ccc(-c2ccc(N(c3ccccc3)c3ccc(-c4ccc5c(c4)sc4ccccc45)cc3)c3c2oc2c4ccccc4c4ccccc4c23)cc1]

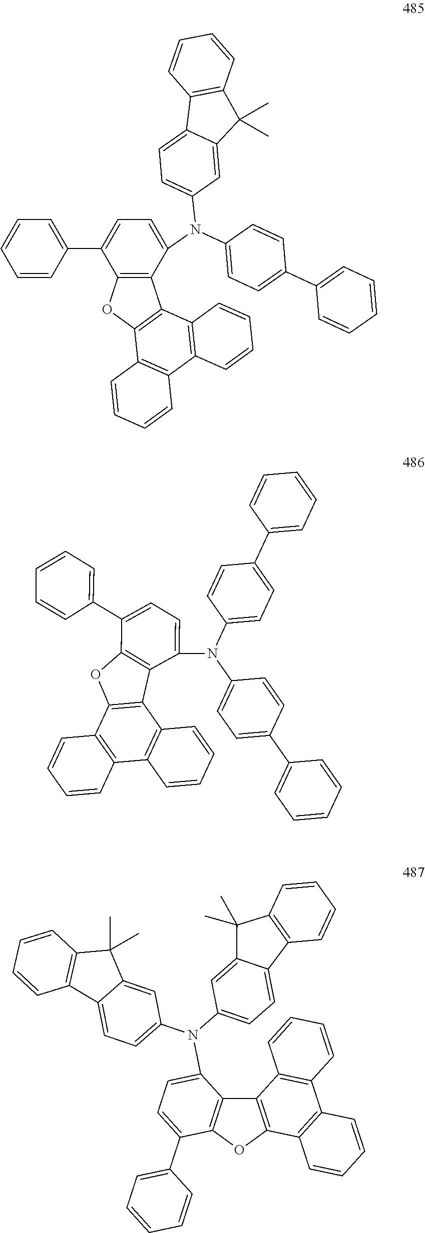 [SMILES: CC1(C)c2ccccc2-c2ccc(N(c3ccc(-c4ccccc4)cc3)c3ccc(-c4ccccc4)c4oc5c6ccccc6c6ccccc6c5c34)cc21 ; c1ccc(-c2ccc(N(c3ccc(-c4ccccc4)cc3)c3ccc(-c4ccccc4)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccccc4)c4oc5c6ccccc6c6ccccc6c5c34)cc21]

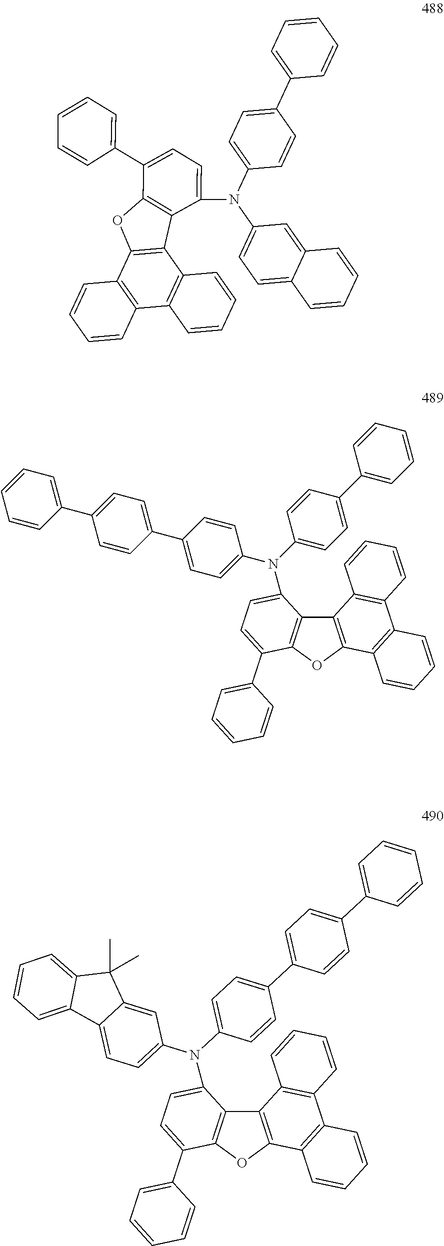 [SMILES: c1ccc(-c2ccc(N(c3ccc4ccccc4c3)c3ccc(-c4ccccc4)c4oc5c6ccccc6c6ccccc6c5c34)cc2)cc1 ; c1ccc(-c2ccc(-c3ccc(N(c4ccc(-c5ccccc5)cc4)c4ccc(-c5ccccc5)c5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3ccc(-c4ccccc4)c4oc5c6ccccc6c6ccccc6c5c34)cc21]

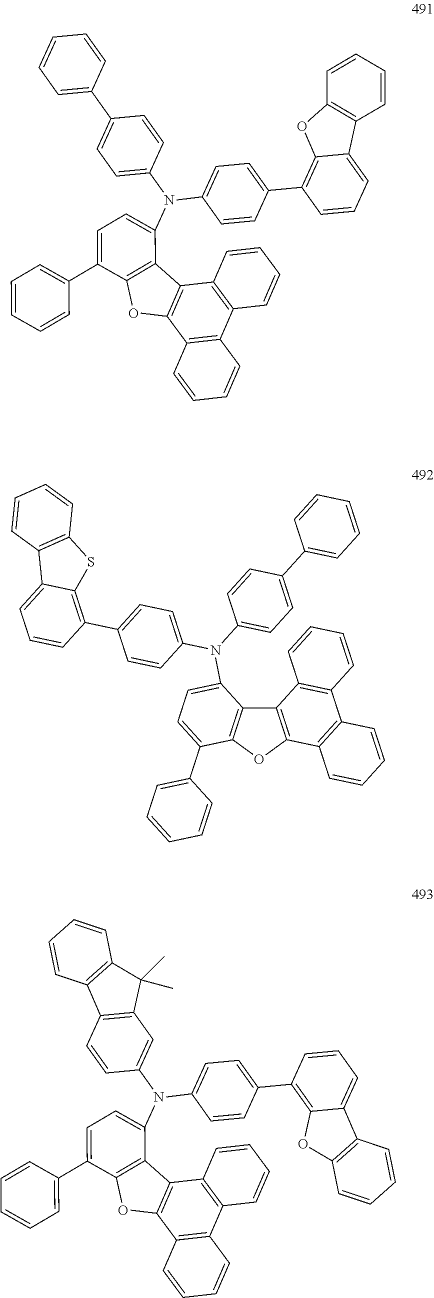 [SMILES: c1ccc(-c2ccc(N(c3ccc(-c4cccc5c4oc4ccccc45)cc3)c3ccc(-c4ccccc4)c4oc5c6ccccc6c6ccccc6c5c34)cc2)cc1 ; c1ccc(-c2ccc(N(c3ccc(-c4cccc5c4sc4ccccc45)cc3)c3ccc(-c4ccccc4)c4oc5c6ccccc6c6ccccc6c5c34)cc2)cc1 ; CC1(C)c2ccccc2-c2ccc(N(c3ccc(-c4cccc5c4oc4ccccc45)cc3)c3ccc(-c4ccccc4)c4oc5c6ccccc6c6ccccc6c5c34)cc21]

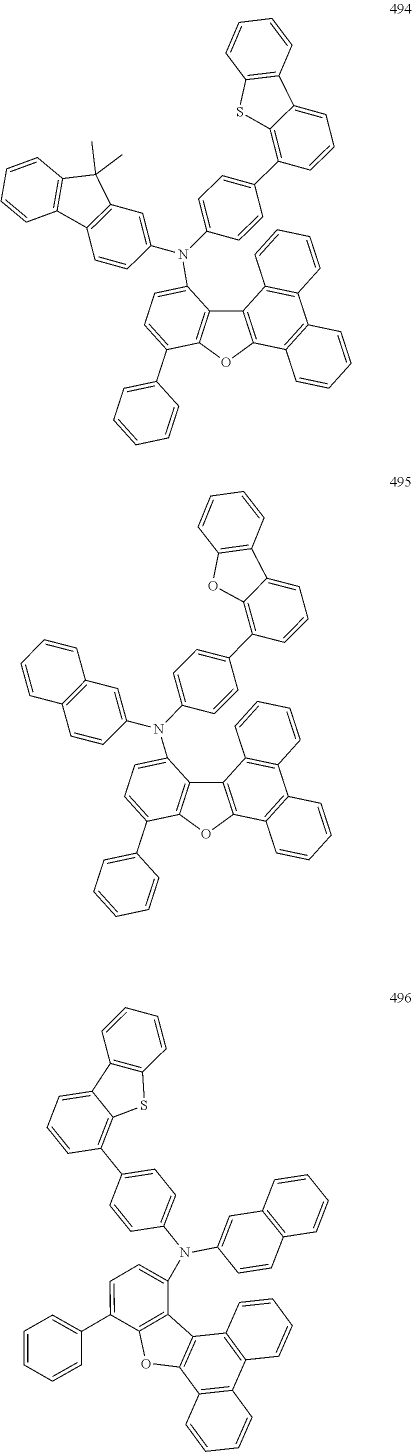 [SMILES: CC1(C)c2ccccc2-c2ccc(N(c3ccc(-c4cccc5c4sc4ccccc45)cc3)c3ccc(-c4ccccc4)c4oc5c6ccccc6c6ccccc6c5c34)cc21 ; c1ccc(-c2ccc(N(c3ccc(-c4cccc5c4oc4ccccc45)cc3)c3ccc4ccccc4c3)c3c2oc2c4ccccc4c4ccccc4c23)cc1 ; c1ccc(-c2ccc(N(c3ccc(-c4cccc5c4sc4ccccc45)cc3)c3ccc4ccccc4c3)c3c2oc2c4ccccc4c4ccccc4c23)cc1]

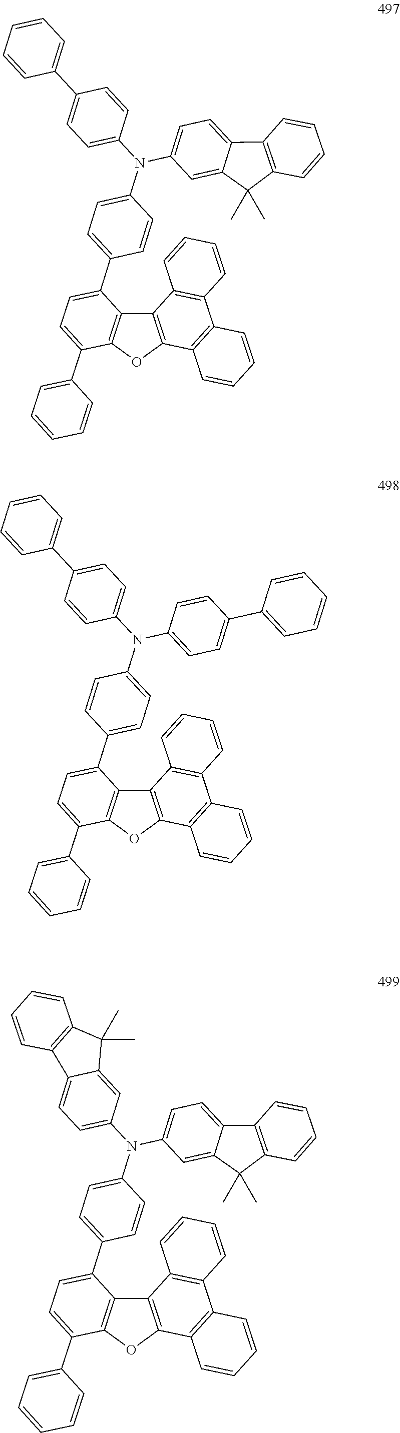 [SMILES: CC1(C)c2ccccc2-c2ccc(N(c3ccc(-c4ccccc4)cc3)c3ccc(-c4ccc(-c5ccccc5)c5oc6c7ccccc7c7ccccc7c6c45)cc3)cc21 ; c1ccc(-c2ccc(N(c3ccc(-c4ccccc4)cc3)c3ccc(-c4ccc(-c5ccccc5)c5oc6c7ccccc7c7ccccc7c6c45)cc3)cc2)cc1 ; CC1(C)c2ccccc2-c2ccc(N(c3ccc(-c4ccc(-c5ccccc5)c5oc6c7ccccc7c7ccccc7c6c45)cc3)c3ccc4c(c3)C(C)(C)c3ccccc3-4)cc21]

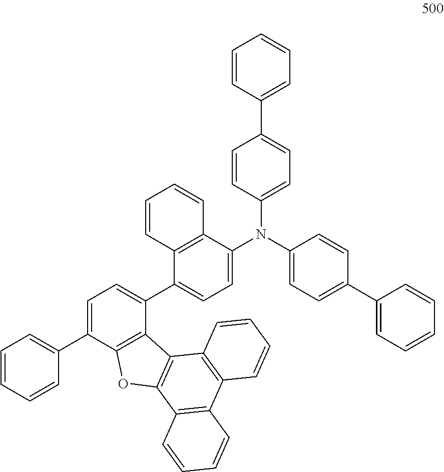 [SMILES: c1ccc(-c2ccc(N(c3ccc(-c4ccccc4)cc3)c3ccc(-c4ccc(-c5ccccc5)c5oc6c7ccccc7c7ccccc7c6c45)c4ccccc34)cc2)cc1]

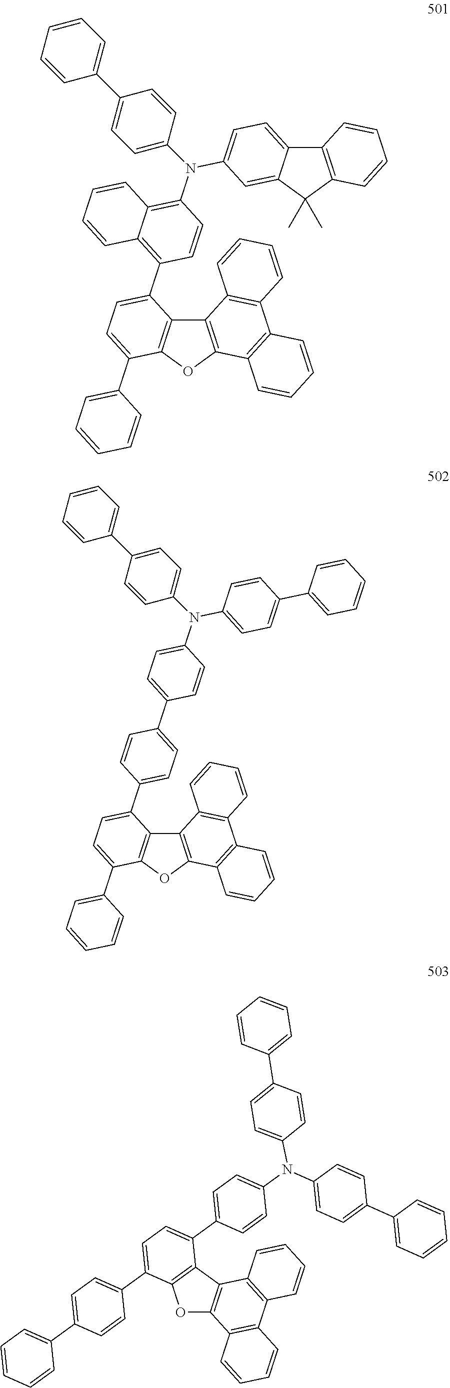

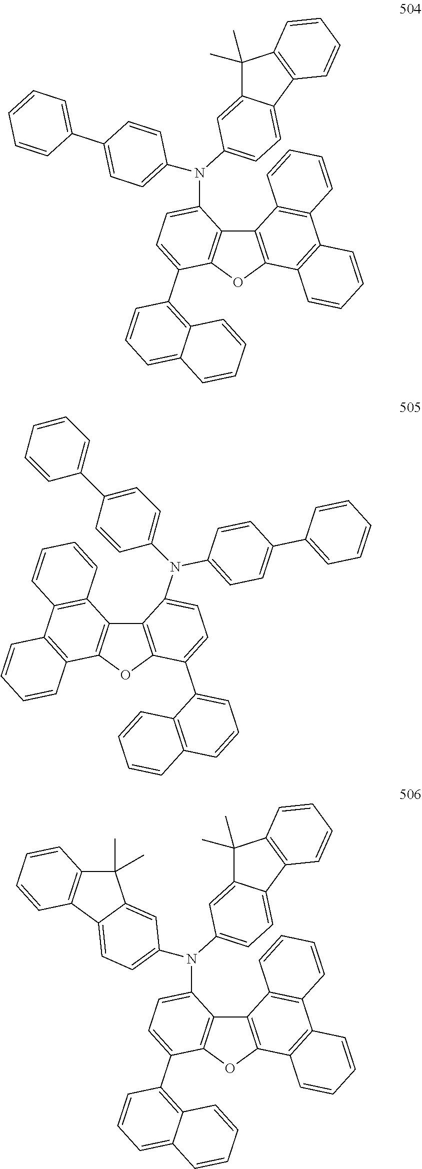 [SMILES: CC1(C)c2ccccc2-c2ccc(N(c3ccc(-c4ccccc4)cc3)c3ccc(-c4cccc5ccccc45)c4oc5c6ccccc6c6ccccc6c5c34)cc21 ; c1ccc(-c2ccc(N(c3ccc(-c4ccccc4)cc3)c3ccc(-c4cccc5ccccc45)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4cccc5ccccc45)c4oc5c6ccccc6c6ccccc6c5c34)cc21]

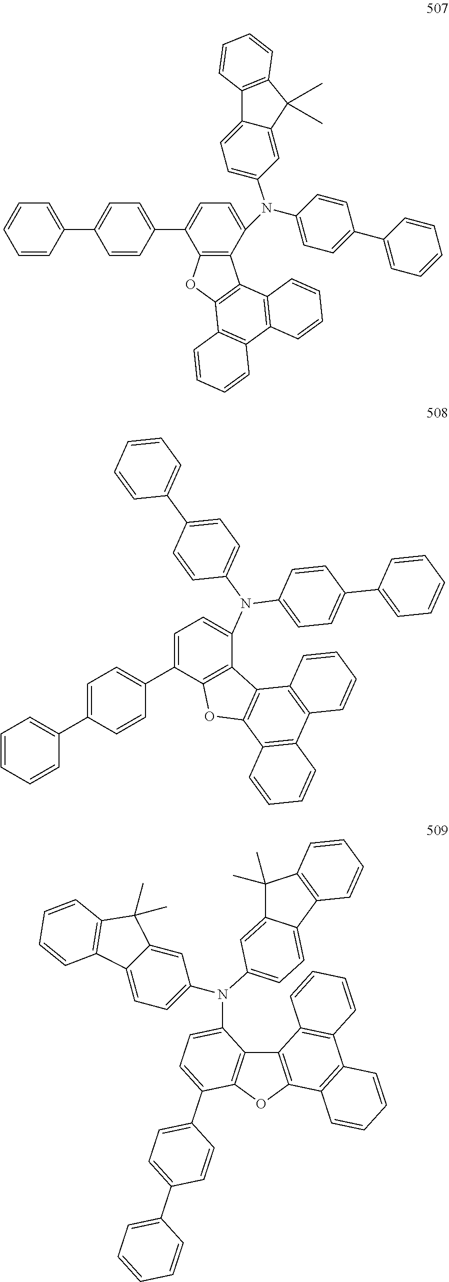

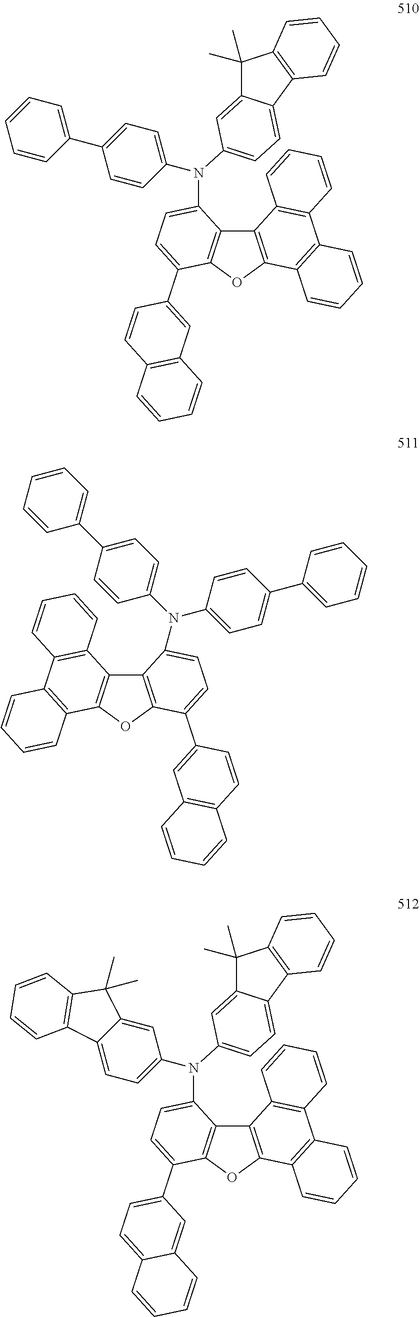 [SMILES: CC1(C)c2ccccc2-c2ccc(N(c3ccc(-c4ccccc4)cc3)c3ccc(-c4ccc5ccccc5c4)c4oc5c6ccccc6c6ccccc6c5c34)cc21 ; c1ccc(-c2ccc(N(c3ccc(-c4ccccc4)cc3)c3ccc(-c4ccc5ccccc5c4)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccc5ccccc5c4)c4oc5c6ccccc6c6ccccc6c5c34)cc21]

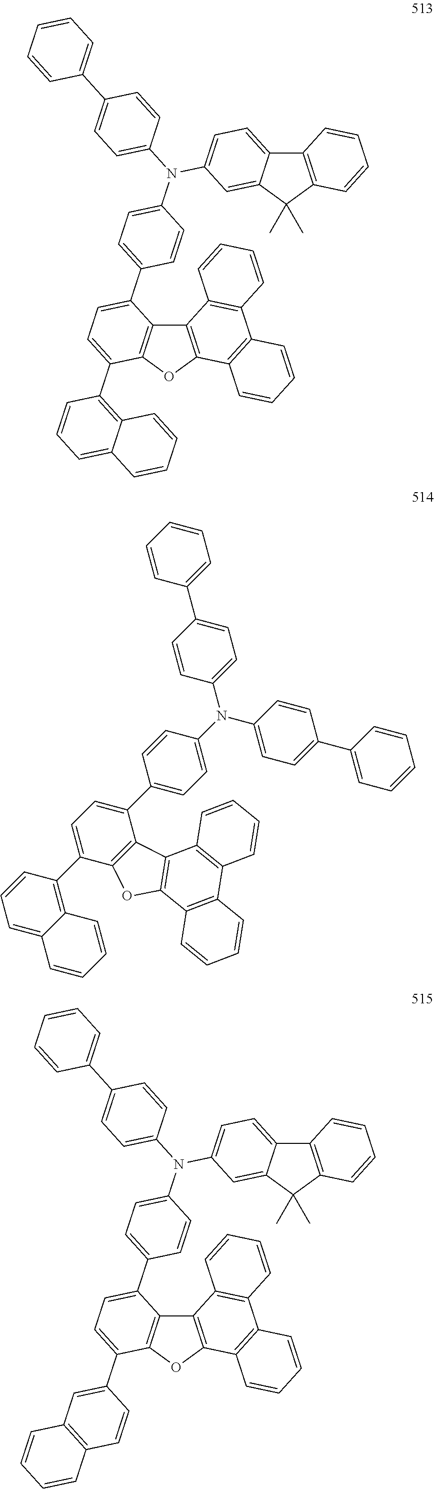

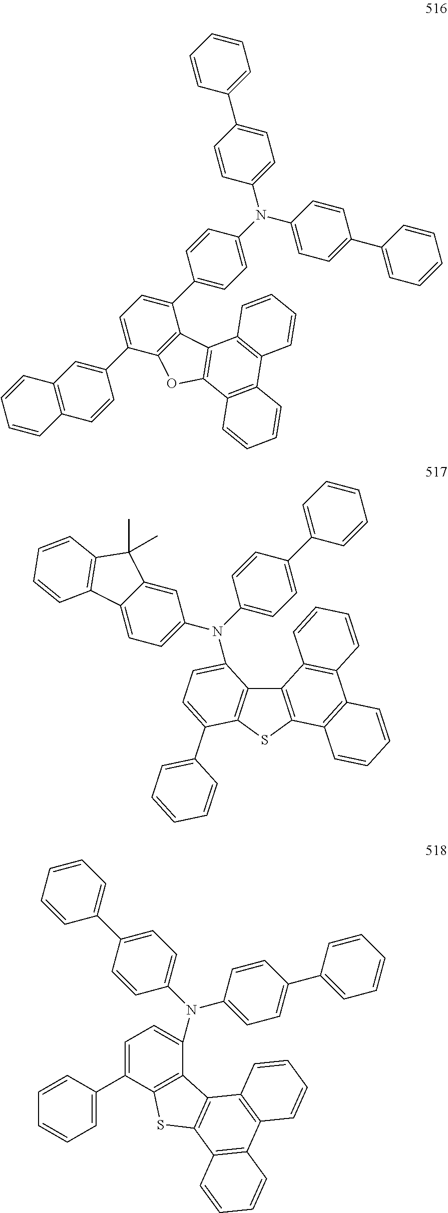 [SMILES: c1ccc(-c2ccc(N(c3ccc(-c4ccccc4)cc3)c3ccc(-c4ccc(-c5ccc6ccccc6c5)c5oc6c7ccccc7c7ccccc7c6c45)cc3)cc2)cc1 ; CC1(C)c2ccccc2-c2ccc(N(c3ccc(-c4ccccc4)cc3)c3ccc(-c4ccccc4)c4sc5c6ccccc6c6ccccc6c5c34)cc21 ; c1ccc(-c2ccc(N(c3ccc(-c4ccccc4)cc3)c3ccc(-c4ccccc4)c4sc5c6ccccc6c6ccccc6c5c34)cc2)cc1]

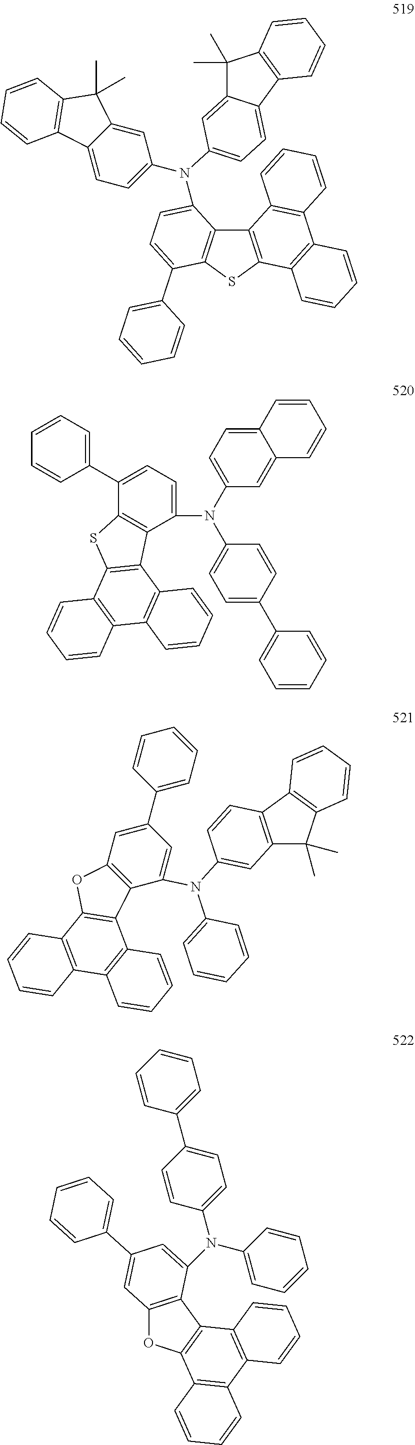

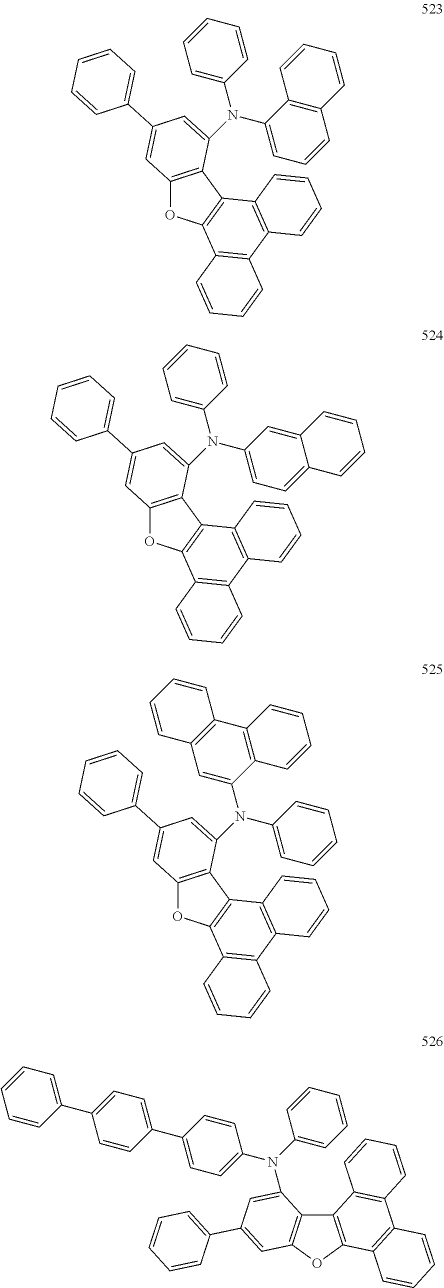

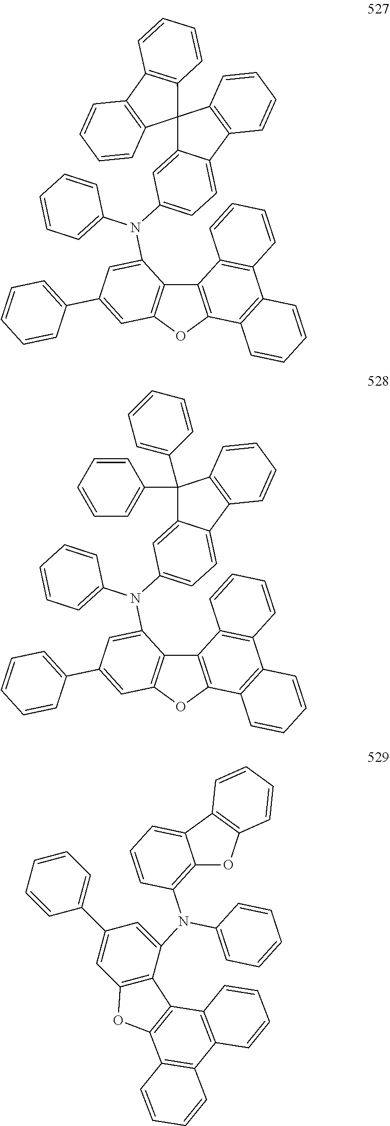

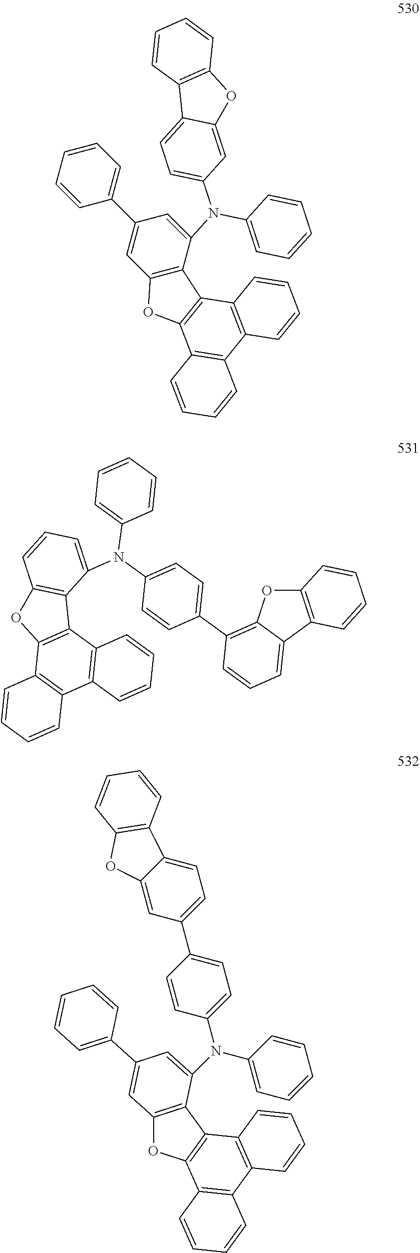 [SMILES: c1ccc(-c2cc(N(c3ccccc3)c3ccc4c(c3)oc3ccccc34)c3c(c2)oc2c4ccccc4c4ccccc4c23)cc1 ; c1ccc(N(c2ccc(-c3cccc4c3oc3ccccc34)cc2)c2cccc3oc4c5ccccc5c5ccccc5c4c23)cc1 ; c1ccc(-c2cc(N(c3ccccc3)c3ccc(-c4ccc5c(c4)oc4ccccc45)cc3)c3c(c2)oc2c4ccccc4c4ccccc4c23)cc1]

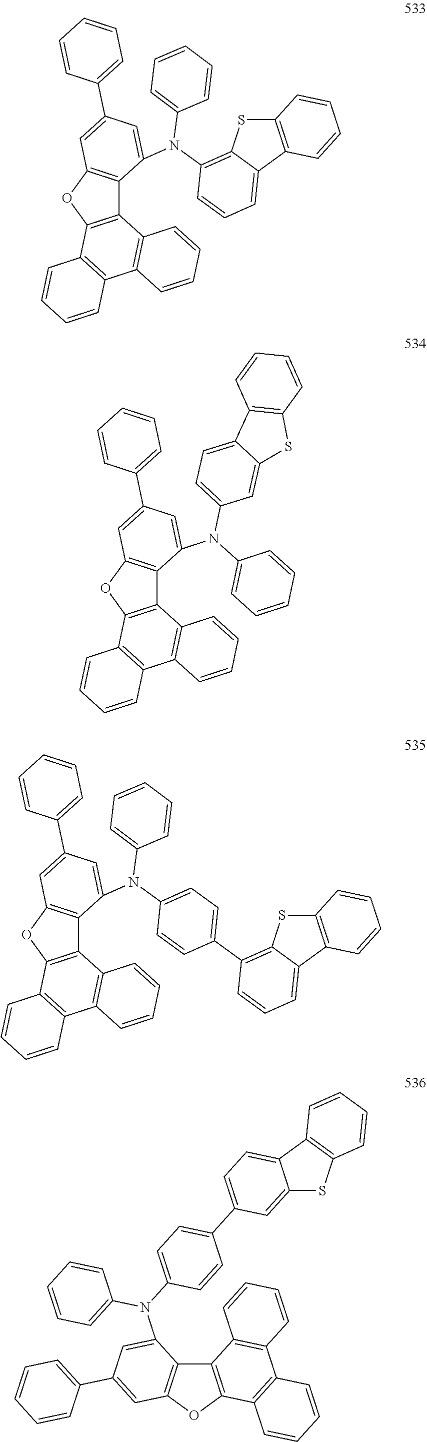 [SMILES: c1ccc(-c2cc(N(c3ccccc3)c3cccc4c3sc3ccccc34)c3c(c2)oc2c4ccccc4c4ccccc4c23)cc1 ; c1ccc(-c2cc(N(c3ccccc3)c3ccc4c(c3)sc3ccccc34)c3c(c2)oc2c4ccccc4c4ccccc4c23)cc1 ; c1ccc(-c2cc(N(c3ccccc3)c3ccc(-c4cccc5c4sc4ccccc45)cc3)c3c(c2)oc2c4ccccc4c4ccccc4c23)cc1 ; c1ccc(-c2cc(N(c3ccccc3)c3ccc(-c4ccc5c(c4)sc4ccccc45)cc3)c3c(c2)oc2c4ccccc4c4ccccc4c23)cc1]

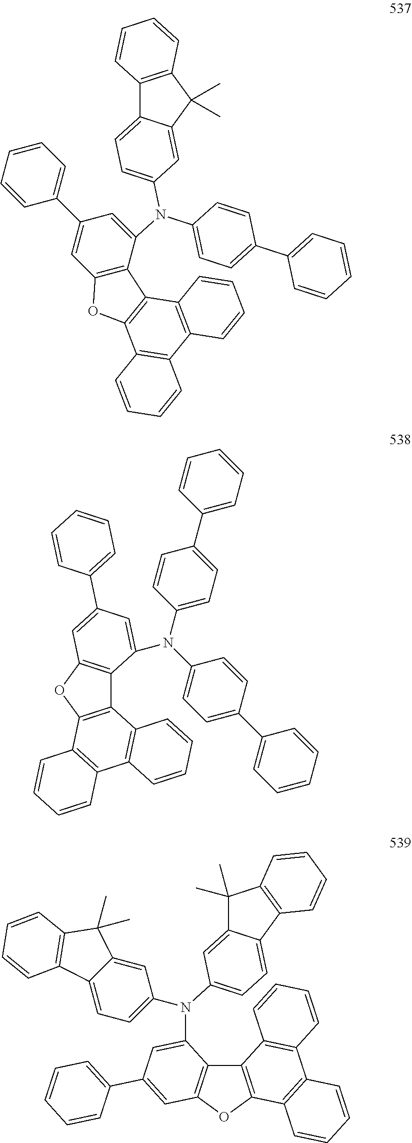 [SMILES: CC1(C)c2ccccc2-c2ccc(N(c3ccc(-c4ccccc4)cc3)c3cc(-c4ccccc4)cc4oc5c6ccccc6c6ccccc6c5c34)cc21 ; c1ccc(-c2ccc(N(c3ccc(-c4ccccc4)cc3)c3cc(-c4ccccc4)cc4oc5c6ccccc6c6ccccc6c5c34)cc2)cc1 ; CC1(C)c2ccccc2-c2ccc(N(c3ccc4c(c3)C(C)(C)c3ccccc3-4)c3cc(-c4ccccc4)cc4oc5c6ccccc6c6ccccc6c5c34)cc21]

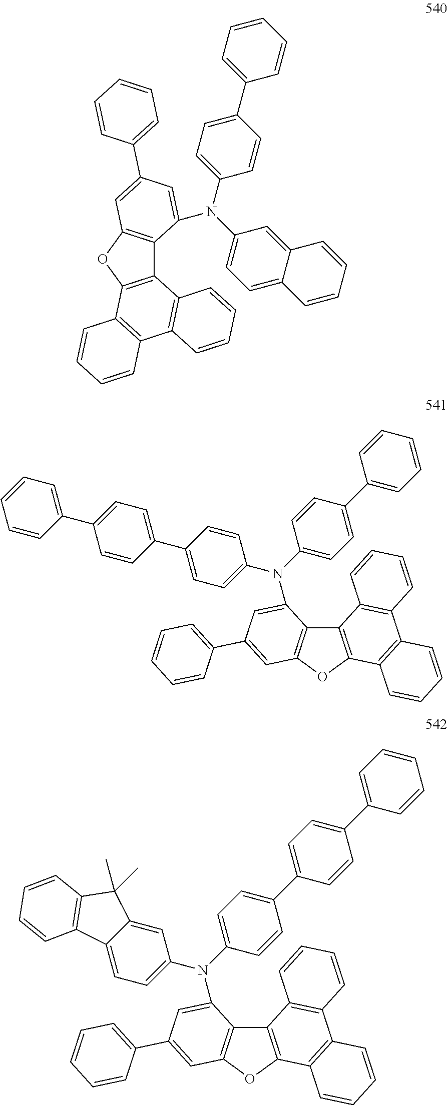 [SMILES: c1ccc(-c2ccc(N(c3ccc4ccccc4c3)c3cc(-c4ccccc4)cc4oc5c6ccccc6c6ccccc6c5c34)cc2)cc1 ; c1ccc(-c2ccc(-c3ccc(N(c4ccc(-c5ccccc5)cc4)c4cc(-c5ccccc5)cc5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3cc(-c4ccccc4)cc4oc5c6ccccc6c6ccccc6c5c34)cc21]

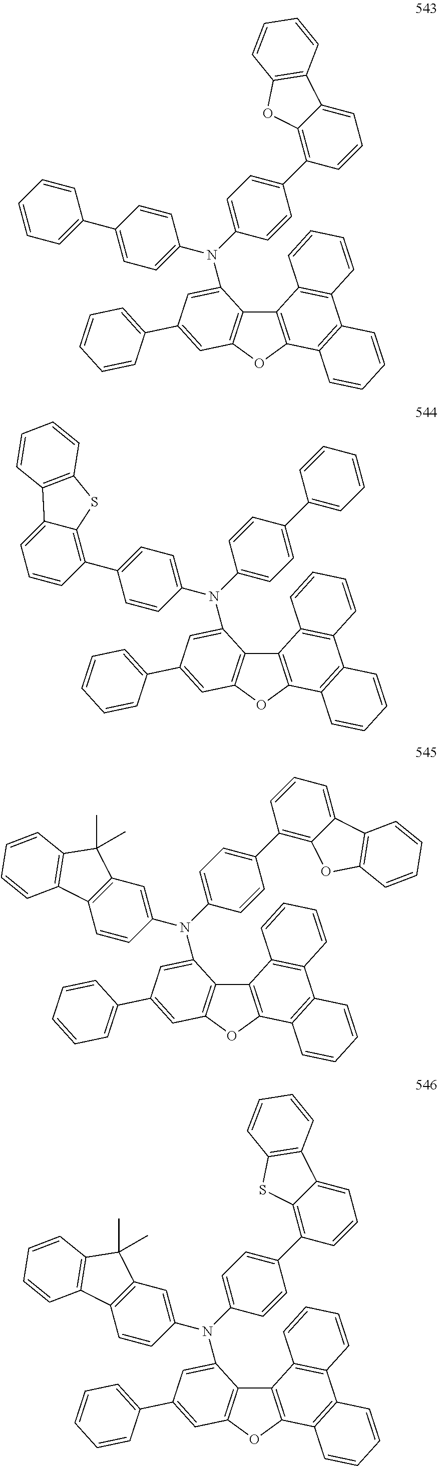

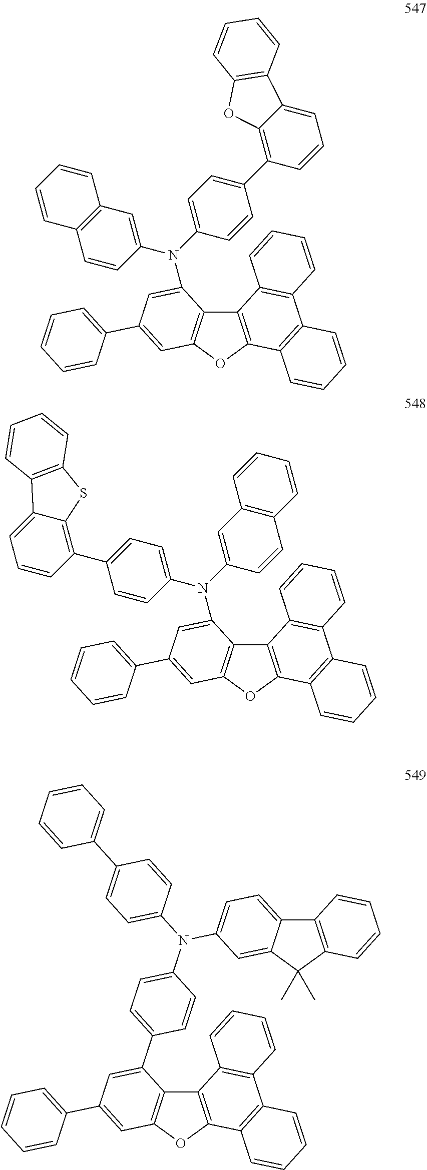 [SMILES: c1ccc(-c2cc(N(c3ccc(-c4cccc5c4oc4ccccc45)cc3)c3ccc4ccccc4c3)c3c(c2)oc2c4ccccc4c4ccccc4c23)cc1 ; c1ccc(-c2cc(N(c3ccc(-c4cccc5c4sc4ccccc45)cc3)c3ccc4ccccc4c3)c3c(c2)oc2c4ccccc4c4ccccc4c23)cc1 ; CC1(C)c2ccccc2-c2ccc(N(c3ccc(-c4ccccc4)cc3)c3ccc(-c4cc(-c5ccccc5)cc5oc6c7ccccc7c7ccccc7c6c45)cc3)cc21]

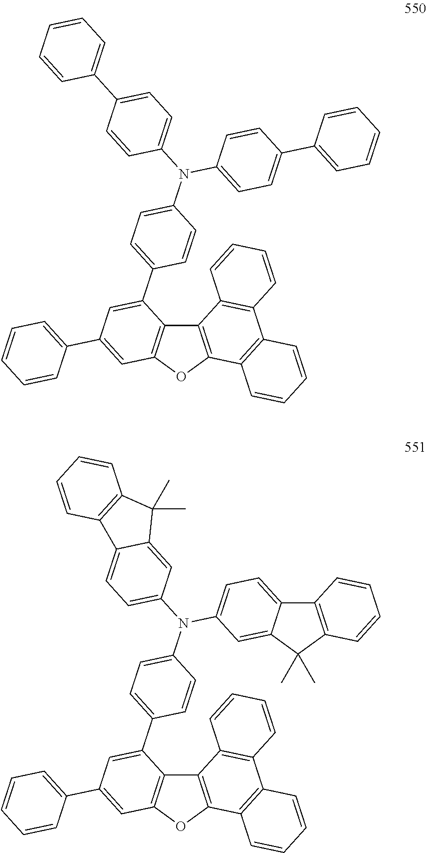 [SMILES: c1ccc(-c2ccc(N(c3ccc(-c4ccccc4)cc3)c3ccc(-c4cc(-c5ccccc5)cc5oc6c7ccccc7c7ccccc7c6c45)cc3)cc2)cc1 ; CC1(C)c2ccccc2-c2ccc(N(c3ccc(-c4cc(-c5ccccc5)cc5oc6c7ccccc7c7ccccc7c6c45)cc3)c3ccc4c(c3)C(C)(C)c3ccccc3-4)cc21]

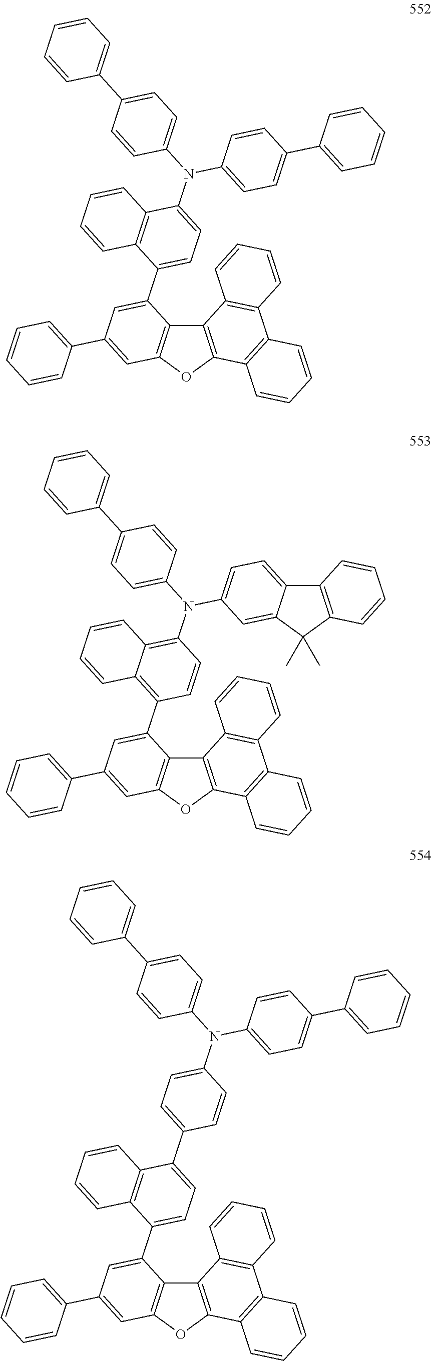 [SMILES: c1ccc(-c2ccc(N(c3ccc(-c4ccccc4)cc3)c3ccc(-c4cc(-c5ccccc5)cc5oc6c7ccccc7c7ccccc7c6c45)c4ccccc34)cc2)cc1 ; CC1(C)c2ccccc2-c2ccc(N(c3ccc(-c4ccccc4)cc3)c3ccc(-c4cc(-c5ccccc5)cc5oc6c7ccccc7c7ccccc7c6c45)c4ccccc34)cc21 ; c1ccc(-c2ccc(N(c3ccc(-c4ccccc4)cc3)c3ccc(-c4ccc(-c5cc(-c6ccccc6)cc6oc7c8ccccc8c8ccccc8c7c56)c5ccccc45)cc3)cc2)cc1]

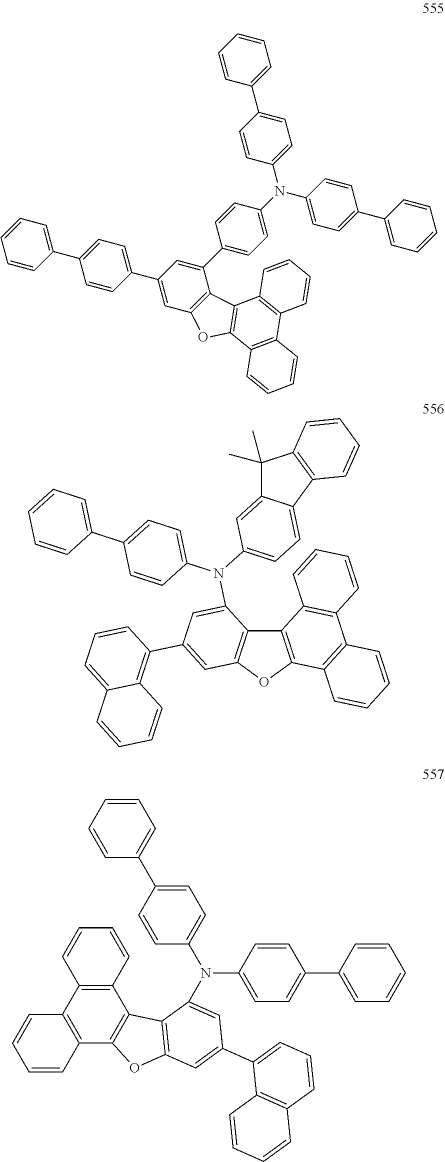

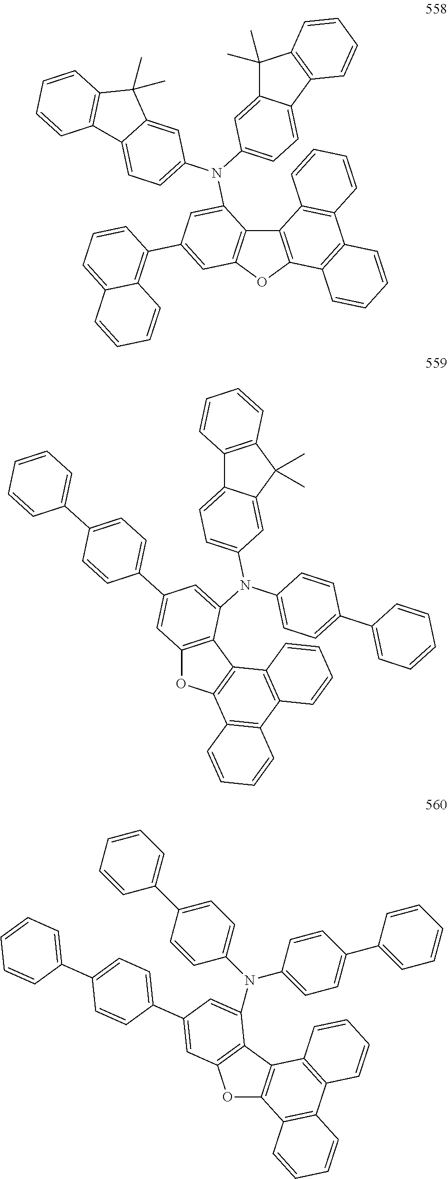 [SMILES: CC1(C)c2ccccc2-c2ccc(N(c3ccc4c(c3)C(C)(C)c3ccccc3-4)c3cc(-c4cccc5ccccc45)cc4oc5c6ccccc6c6ccccc6c5c34)cc21 ; CC1(C)c2ccccc2-c2ccc(N(c3ccc(-c4ccccc4)cc3)c3cc(-c4ccc(-c5ccccc5)cc4)cc4oc5c6ccccc6c6ccccc6c5c34)cc21 ; c1ccc(-c2ccc(-c3cc(N(c4ccc(-c5ccccc5)cc4)c4ccc(-c5ccccc5)cc4)c4c(c3)oc3c5ccccc5c5ccccc5c34)cc2)cc1]

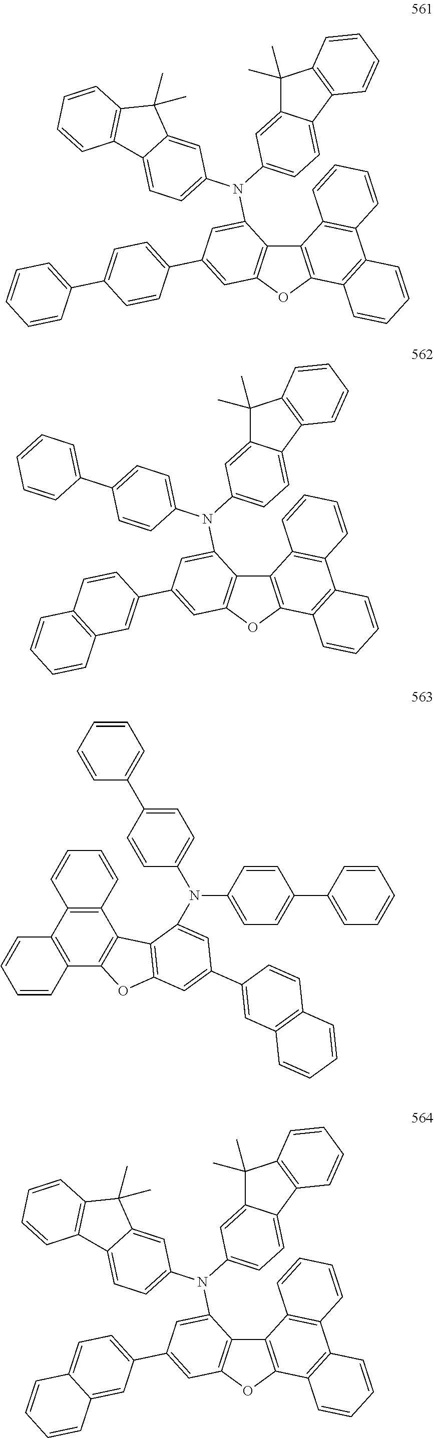 [SMILES: CC1(C)c2ccccc2-c2ccc(N(c3ccc4c(c3)C(C)(C)c3ccccc3-4)c3cc(-c4ccc(-c5ccccc5)cc4)cc4oc5c6ccccc6c6ccccc6c5c34)cc21 ; CC1(C)c2ccccc2-c2ccc(N(c3ccc(-c4ccccc4)cc3)c3cc(-c4ccc5ccccc5c4)cc4oc5c6ccccc6c6ccccc6c5c34)cc21 ; c1ccc(-c2ccc(N(c3ccc(-c4ccccc4)cc3)c3cc(-c4ccc5ccccc5c4)cc4oc5c6ccccc6c6ccccc6c5c34)cc2)cc1 ; CC1(C)c2ccccc2-c2ccc(N(c3ccc4c(c3)C(C)(C)c3ccccc3-4)c3cc(-c4ccc5ccccc5c4)cc4oc5c6ccccc6c6ccccc6c5c34)cc21]

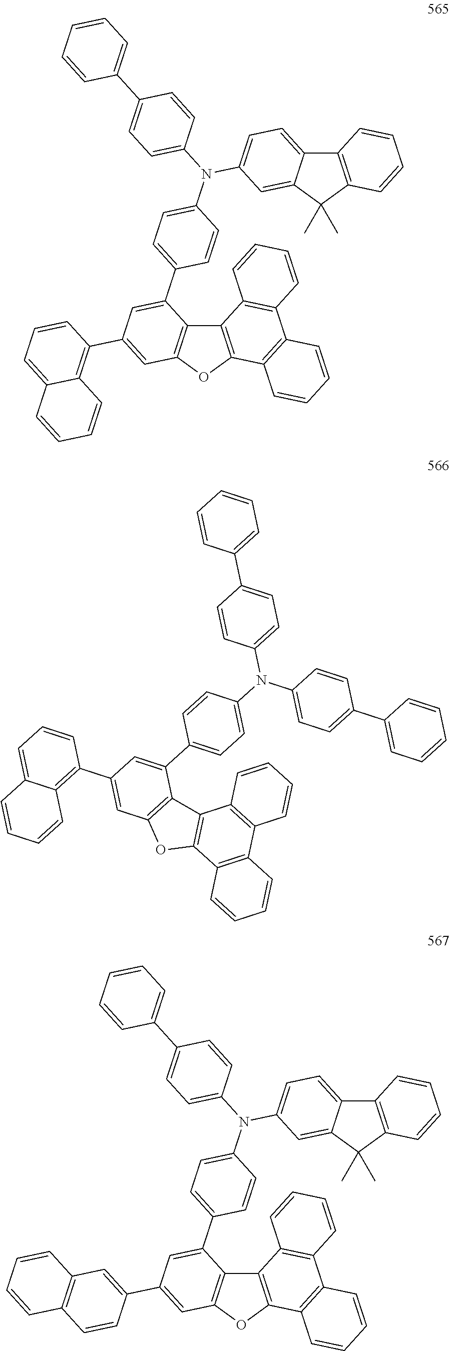 [SMILES: CC1(C)c2ccccc2-c2ccc(N(c3ccc(-c4ccccc4)cc3)c3ccc(-c4cc(-c5cccc6ccccc56)cc5oc6c7ccccc7c7ccccc7c6c45)cc3)cc21 ; c1ccc(-c2ccc(N(c3ccc(-c4ccccc4)cc3)c3ccc(-c4cc(-c5cccc6ccccc56)cc5oc6c7ccccc7c7ccccc7c6c45)cc3)cc2)cc1 ; CC1(C)c2ccccc2-c2ccc(N(c3ccc(-c4ccccc4)cc3)c3ccc(-c4cc(-c5ccc6ccccc6c5)cc5oc6c7ccccc7c7ccccc7c6c45)cc3)cc21]

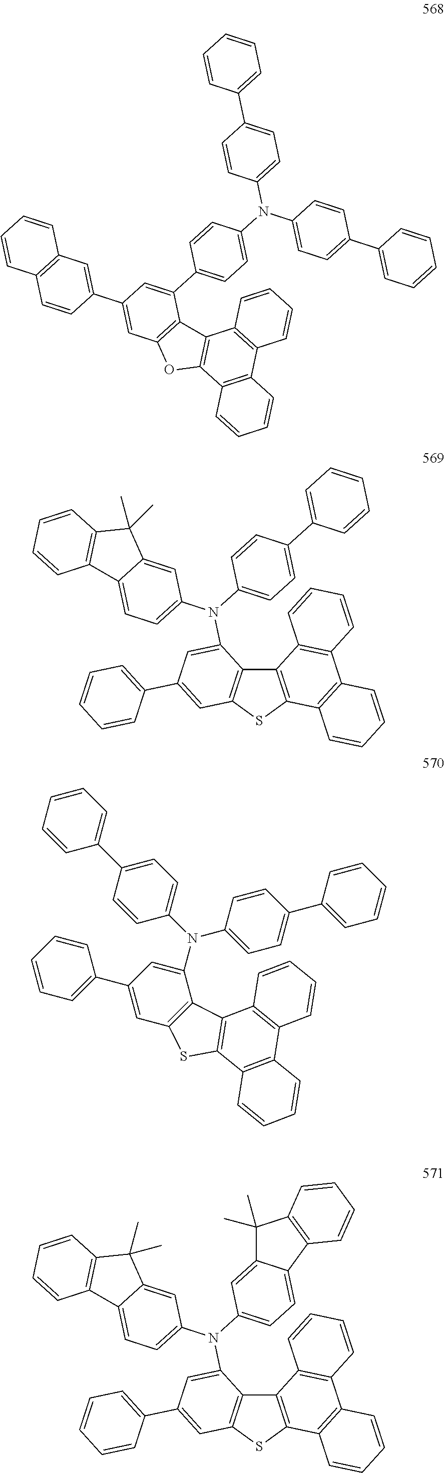 [SMILES: c1ccc(-c2ccc(N(c3ccc(-c4ccccc4)cc3)c3ccc(-c4cc(-c5ccc6ccccc6c5)cc5oc6c7ccccc7c7ccccc7c6c45)cc3)cc2)cc1 ; CC1(C)c2ccccc2-c2ccc(N(c3ccc(-c4ccccc4)cc3)c3cc(-c4ccccc4)cc4sc5c6ccccc6c6ccccc6c5c34)cc21 ; c1ccc(-c2ccc(N(c3ccc(-c4ccccc4)cc3)c3cc(-c4ccccc4)cc4sc5c6ccccc6c6ccccc6c5c34)cc2)cc1 ; CC1(C)c2ccccc2-c2ccc(N(c3ccc4c(c3)C(C)(C)c3ccccc3-4)c3cc(-c4ccccc4)cc4sc5c6ccccc6c6ccccc6c5c34)cc21]

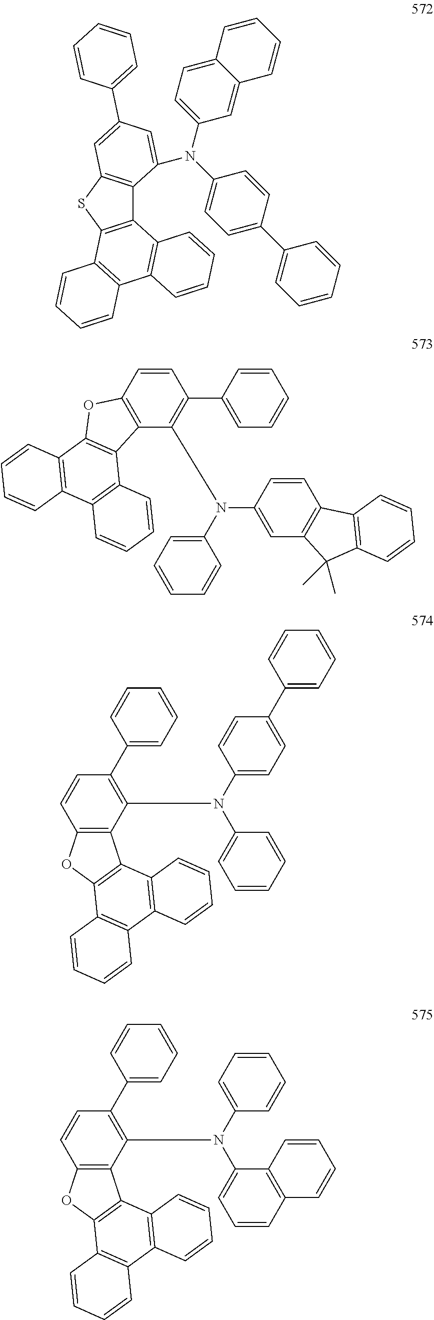

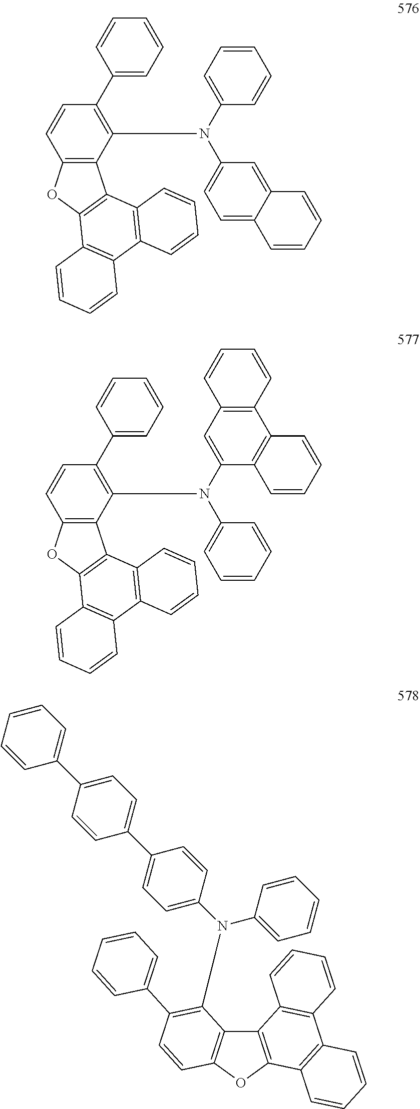 [SMILES: c1ccc(-c2ccc3oc4c5ccccc5c5ccccc5c4c3c2N(c2ccccc2)c2ccc3ccccc3c2)cc1 ; c1ccc(-c2ccc3oc4c5ccccc5c5ccccc5c4c3c2N(c2ccccc2)c2cc3ccccc3c3ccccc23)cc1 ; c1ccc(-c2ccc(-c3ccc(N(c4ccccc4)c4c(-c5ccccc5)ccc5oc6c7ccccc7c7ccccc7c6c45)cc3)cc2)cc1]

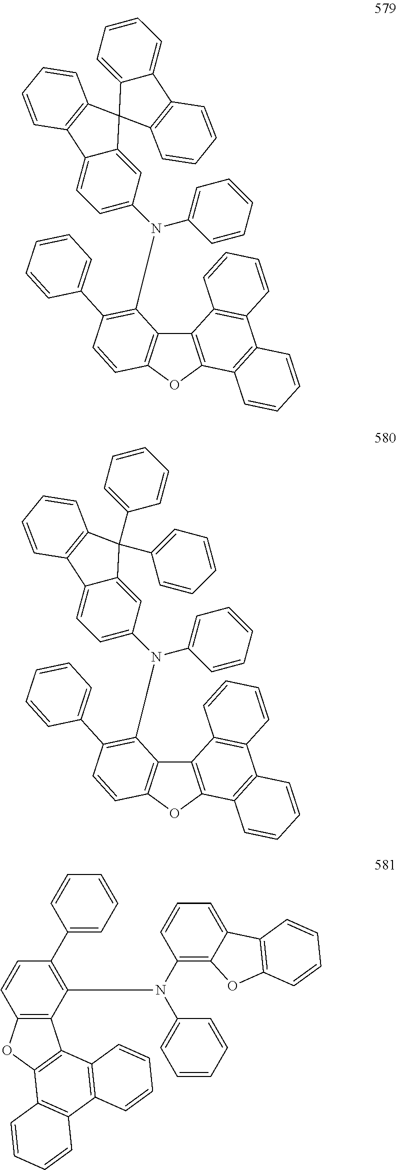 [SMILES: c1ccc(-c2ccc3oc4c5ccccc5c5ccccc5c4c3c2N(c2ccccc2)c2ccc3c(c2)C2(c4ccccc4-c4ccccc42)c2ccccc2-3)cc1 ; c1ccc(-c2ccc3oc4c5ccccc5c5ccccc5c4c3c2N(c2ccccc2)c2ccc3c(c2)C(c2ccccc2)(c2ccccc2)c2ccccc2-3)cc1 ; c1ccc(-c2ccc3oc4c5ccccc5c5ccccc5c4c3c2N(c2ccccc2)c2cccc3c2oc2ccccc23)cc1]

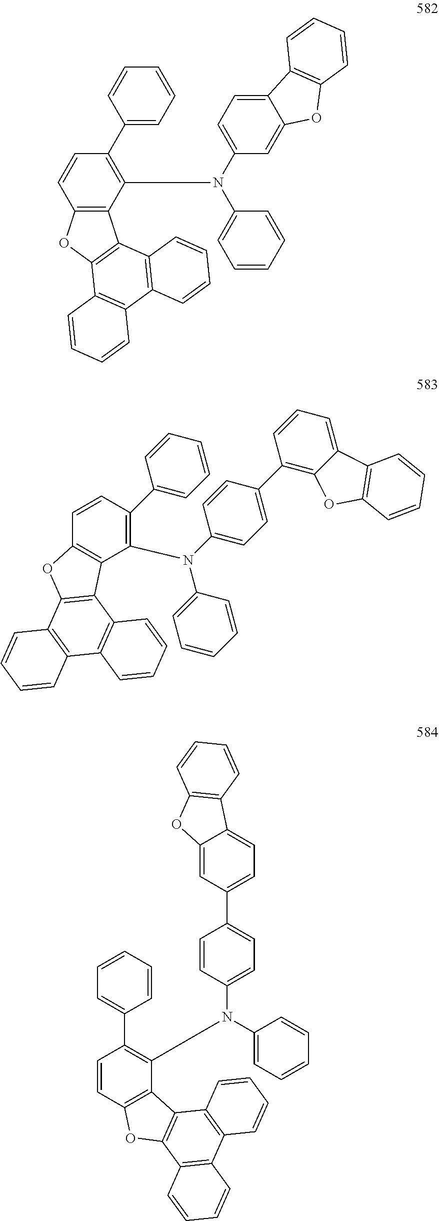 [SMILES: c1ccc(-c2ccc3oc4c5ccccc5c5ccccc5c4c3c2N(c2ccccc2)c2ccc3c(c2)oc2ccccc23)cc1 ; c1ccc(-c2ccc3oc4c5ccccc5c5ccccc5c4c3c2N(c2ccccc2)c2ccc(-c3cccc4c3oc3ccccc34)cc2)cc1 ; c1ccc(-c2ccc3oc4c5ccccc5c5ccccc5c4c3c2N(c2ccccc2)c2ccc(-c3ccc4c(c3)oc3ccccc34)cc2)cc1]

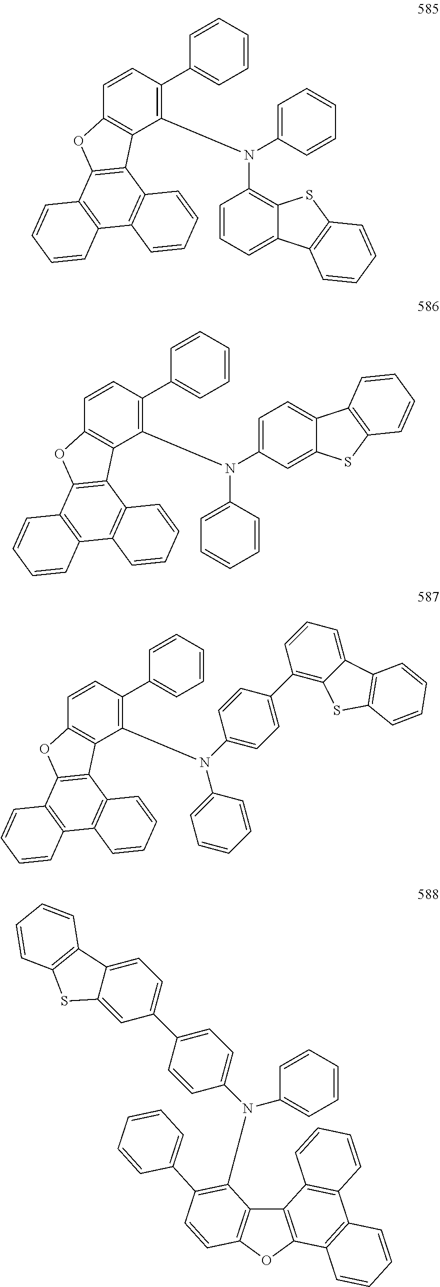

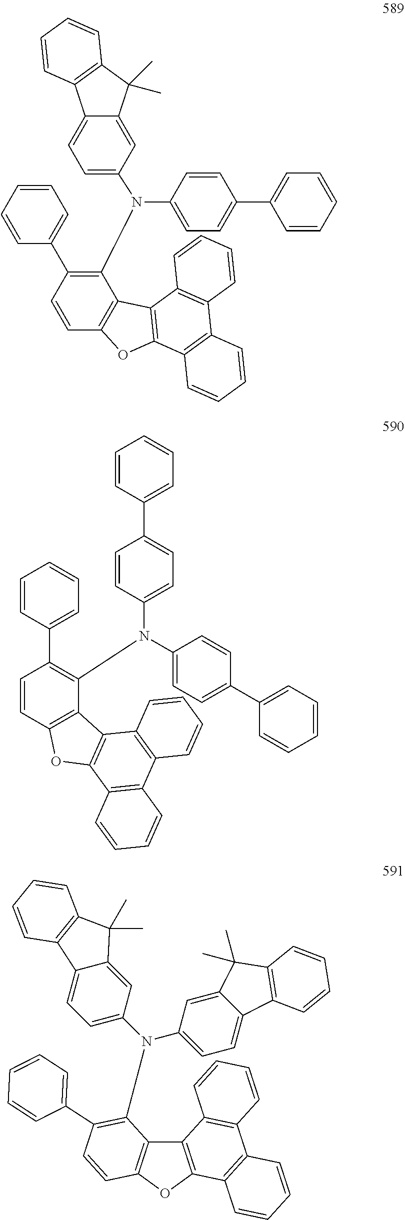 [SMILES: CC1(C)c2ccccc2-c2ccc(N(c3ccc(-c4ccccc4)cc3)c3c(-c4ccccc4)ccc4oc5c6ccccc6c6ccccc6c5c34)cc21 ; c1ccc(-c2ccc(N(c3ccc(-c4ccccc4)cc3)c3c(-c4ccccc4)ccc4oc5c6ccccc6c6ccccc6c5c34)cc2)cc1 ; CC1(C)c2ccccc2-c2ccc(N(c3ccc4c(c3)C(C)(C)c3ccccc3-4)c3c(-c4ccccc4)ccc4oc5c6ccccc6c6ccccc6c5c34)cc21]

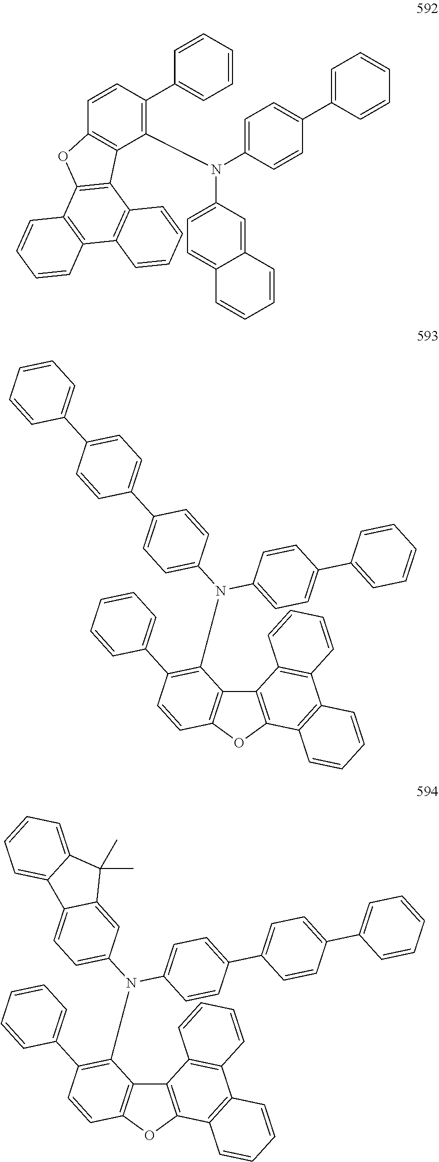 [SMILES: c1ccc(-c2ccc(N(c3ccc4ccccc4c3)c3c(-c4ccccc4)ccc4oc5c6ccccc6c6ccccc6c5c34)cc2)cc1 ; c1ccc(-c2ccc(-c3ccc(N(c4ccc(-c5ccccc5)cc4)c4c(-c5ccccc5)ccc5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3c(-c4ccccc4)ccc4oc5c6ccccc6c6ccccc6c5c34)cc21]

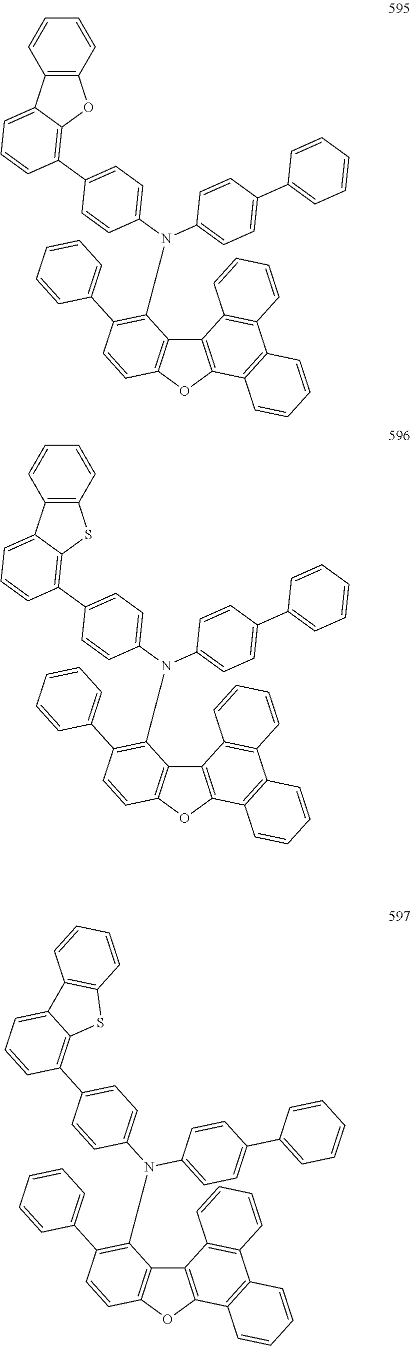 [SMILES: c1ccc(-c2ccc(N(c3ccc(-c4cccc5c4oc4ccccc45)cc3)c3c(-c4ccccc4)ccc4oc5c6ccccc6c6ccccc6c5c34)cc2)cc1 ; c1ccc(-c2ccc(N(c3ccc(-c4cccc5c4sc4ccccc45)cc3)c3c(-c4ccccc4)ccc4oc5c6ccccc6c6ccccc6c5c34)cc2)cc1 ; c1ccc(-c2ccc(N(c3ccc(-c4cccc5c4sc4ccccc45)cc3)c3c(-c4ccccc4)ccc4oc5c6ccccc6c6ccccc6c5c34)cc2)cc1]

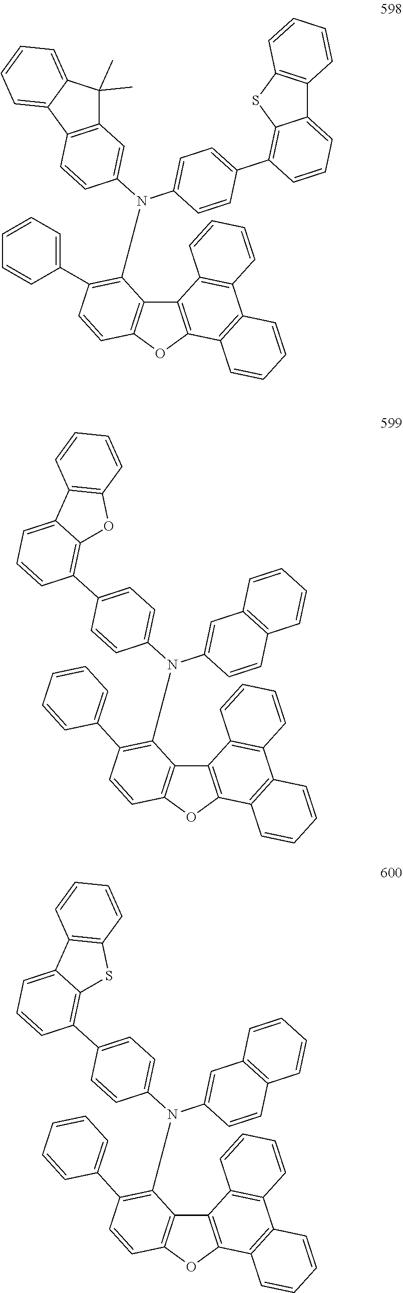 [SMILES: CC1(C)c2ccccc2-c2ccc(N(c3ccc(-c4cccc5c4sc4ccccc45)cc3)c3c(-c4ccccc4)ccc4oc5c6ccccc6c6ccccc6c5c34)cc21 ; c1ccc(-c2ccc3oc4c5ccccc5c5ccccc5c4c3c2N(c2ccc(-c3cccc4c3oc3ccccc34)cc2)c2ccc3ccccc3c2)cc1 ; c1ccc(-c2ccc3oc4c5ccccc5c5ccccc5c4c3c2N(c2ccc(-c3cccc4c3sc3ccccc34)cc2)c2ccc3ccccc3c2)cc1]

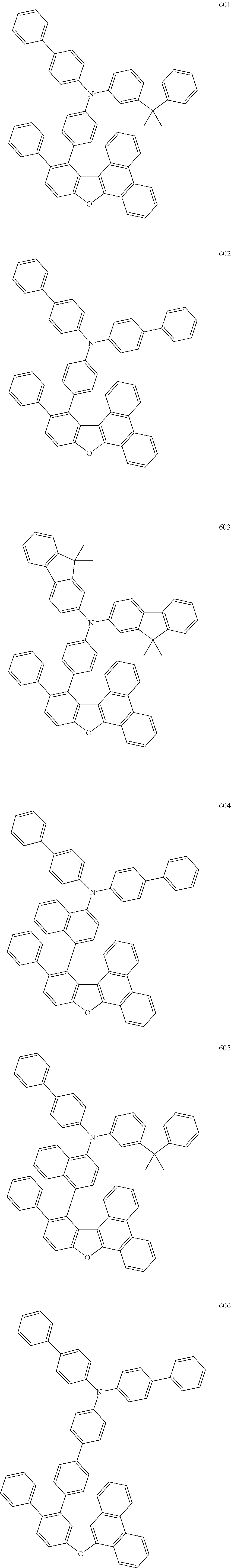

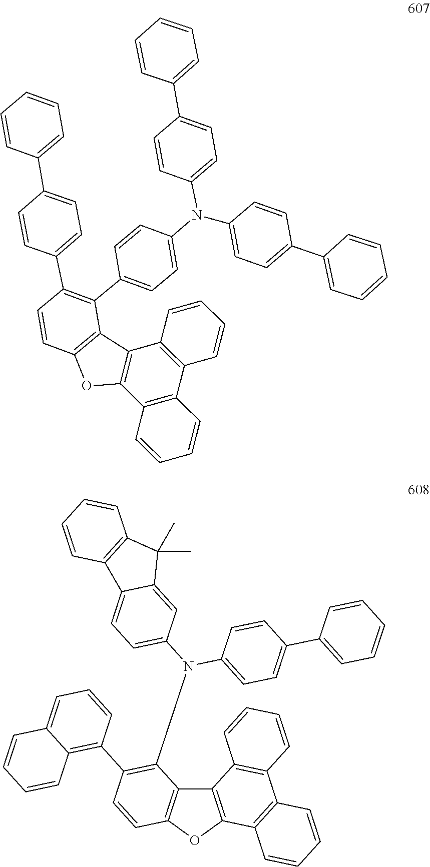 [SMILES: c1ccc(-c2ccc(-c3ccc4oc5c6ccccc6c6ccccc6c5c4c3-c3ccc(N(c4ccc(-c5ccccc5)cc4)c4ccc(-c5ccccc5)cc4)cc3)cc2)cc1 ; CC1(C)c2ccccc2-c2ccc(N(c3ccc(-c4ccccc4)cc3)c3c(-c4cccc5ccccc45)ccc4oc5c6ccccc6c6ccccc6c5c34)cc21]

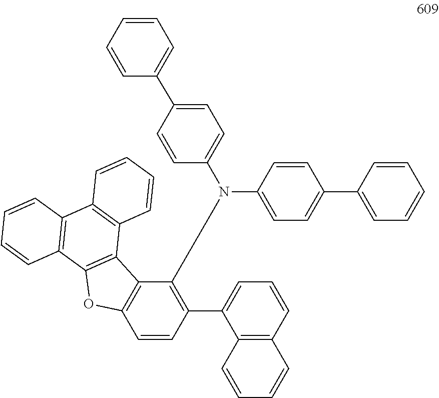 [SMILES: c1ccc(-c2ccc(N(c3ccc(-c4ccccc4)cc3)c3c(-c4cccc5ccccc45)ccc4oc5c6ccccc6c6ccccc6c5c34)cc2)cc1]

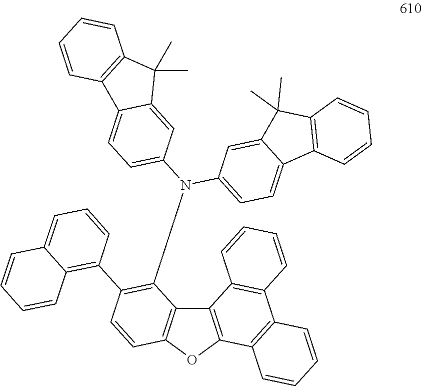 [SMILES: CC1(C)c2ccccc2-c2ccc(N(c3ccc4c(c3)C(C)(C)c3ccccc3-4)c3c(-c4cccc5ccccc45)ccc4oc5c6ccccc6c6ccccc6c5c34)cc21]

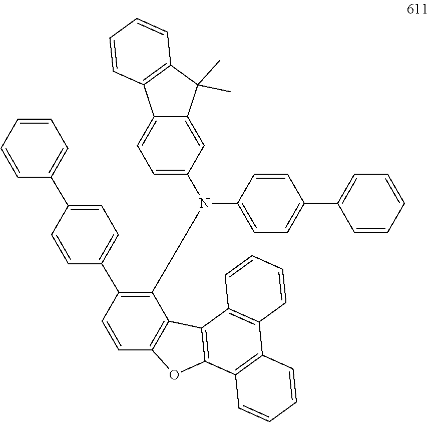 [SMILES: CC1(C)c2ccccc2-c2ccc(N(c3ccc(-c4ccccc4)cc3)c3c(-c4ccc(-c5ccccc5)cc4)ccc4oc5c6ccccc6c6ccccc6c5c34)cc21]

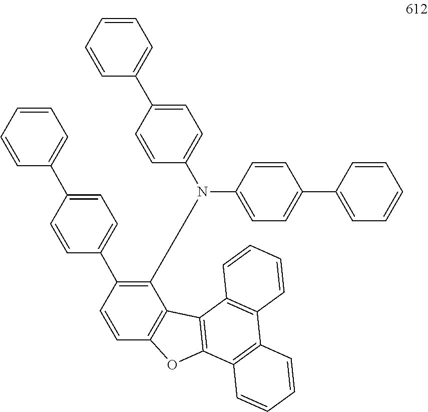 [SMILES: c1ccc(-c2ccc(-c3ccc4oc5c6ccccc6c6ccccc6c5c4c3N(c3ccc(-c4ccccc4)cc3)c3ccc(-c4ccccc4)cc3)cc2)cc1]

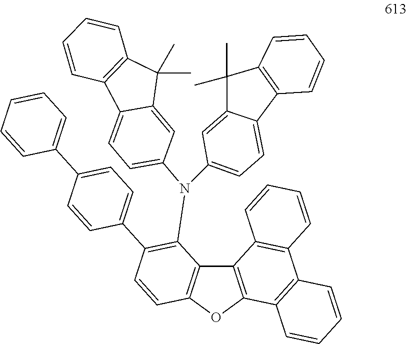 [SMILES: CC1(C)c2ccccc2-c2ccc(N(c3ccc4c(c3)C(C)(C)c3ccccc3-4)c3c(-c4ccc(-c5ccccc5)cc4)ccc4oc5c6ccccc6c6ccccc6c5c34)cc21]

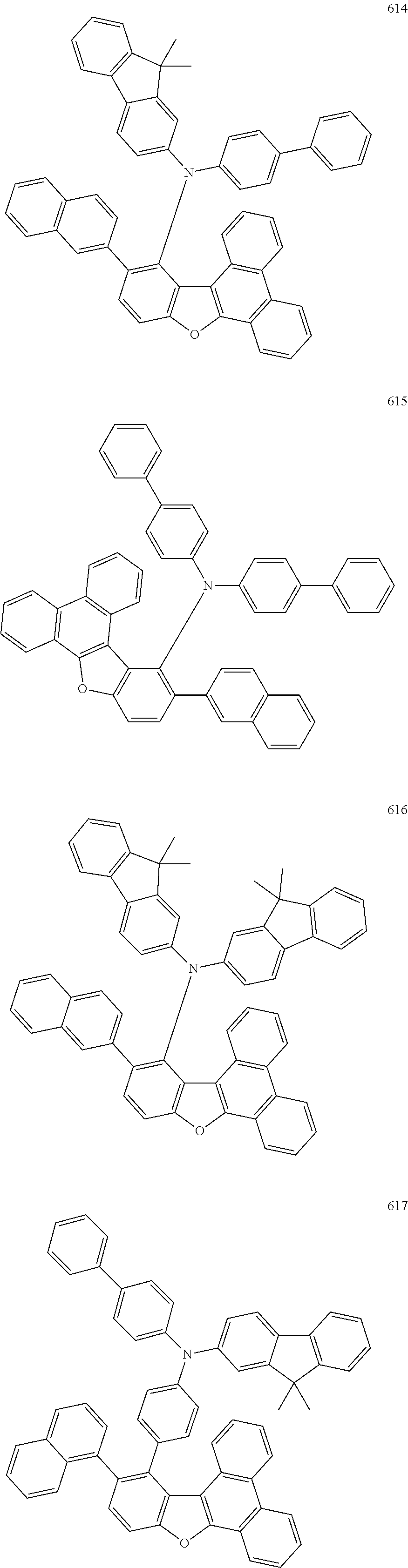 [SMILES: CC1(C)c2ccccc2-c2ccc(N(c3ccc(-c4ccccc4)cc3)c3c(-c4ccc5ccccc5c4)ccc4oc5c6ccccc6c6ccccc6c5c34)cc21 ; c1ccc(-c2ccc(N(c3ccc(-c4ccccc4)cc3)c3c(-c4ccc5ccccc5c4)ccc4oc5c6ccccc6c6ccccc6c5c34)cc2)cc1 ; CC1(C)c2ccccc2-c2ccc(N(c3ccc4c(c3)C(C)(C)c3ccccc3-4)c3c(-c4ccc5ccccc5c4)ccc4oc5c6ccccc6c6ccccc6c5c34)cc21 ; CC1(C)c2ccccc2-c2ccc(N(c3ccc(-c4ccccc4)cc3)c3ccc(-c4c(-c5cccc6ccccc56)ccc5oc6c7ccccc7c7ccccc7c6c45)cc3)cc21]

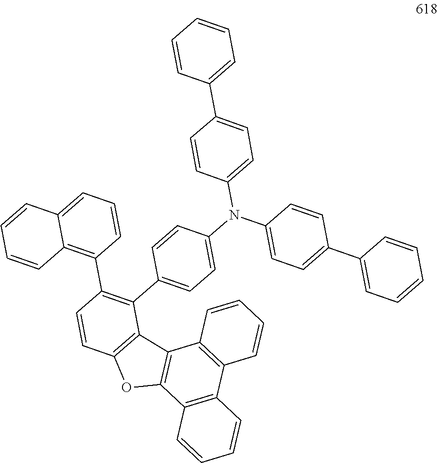 [SMILES: c1ccc(-c2ccc(N(c3ccc(-c4ccccc4)cc3)c3ccc(-c4c(-c5cccc6ccccc56)ccc5oc6c7ccccc7c7ccccc7c6c45)cc3)cc2)cc1]

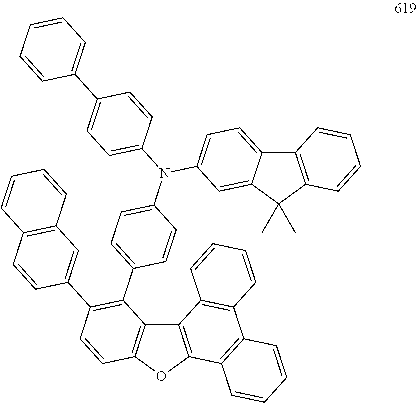 [SMILES: CC1(C)c2ccccc2-c2ccc(N(c3ccc(-c4ccccc4)cc3)c3ccc(-c4c(-c5ccc6ccccc6c5)ccc5oc6c7ccccc7c7ccccc7c6c45)cc3)cc21]

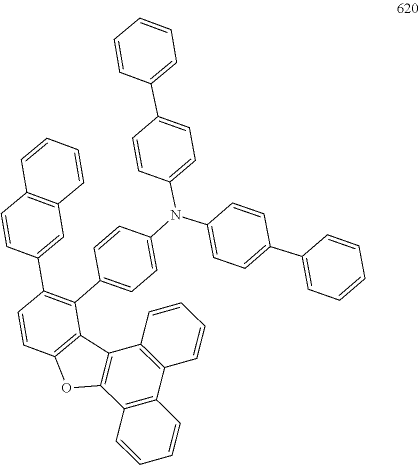 [SMILES: c1ccc(-c2ccc(N(c3ccc(-c4ccccc4)cc3)c3ccc(-c4c(-c5ccc6ccccc6c5)ccc5oc6c7ccccc7c7ccccc7c6c45)cc3)cc2)cc1]

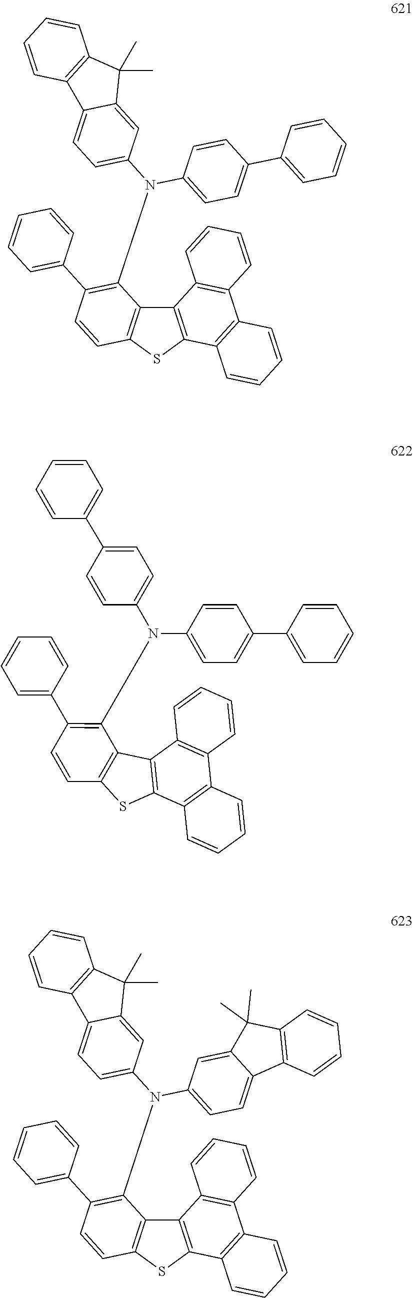 [SMILES: CC1(C)c2ccccc2-c2ccc(N(c3ccc(-c4ccccc4)cc3)c3c(-c4ccccc4)ccc4sc5c6ccccc6c6ccccc6c5c34)cc21 ; c1ccc(-c2ccc(N(c3ccc(-c4ccccc4)cc3)c3c(-c4ccccc4)ccc4sc5c6ccccc6c6ccccc6c5c34)cc2)cc1 ; CC1(C)c2ccccc2-c2ccc(N(c3ccc4c(c3)C(C)(C)c3ccccc3-4)c3c(-c4ccccc4)ccc4sc5c6ccccc6c6ccccc6c5c34)cc21]

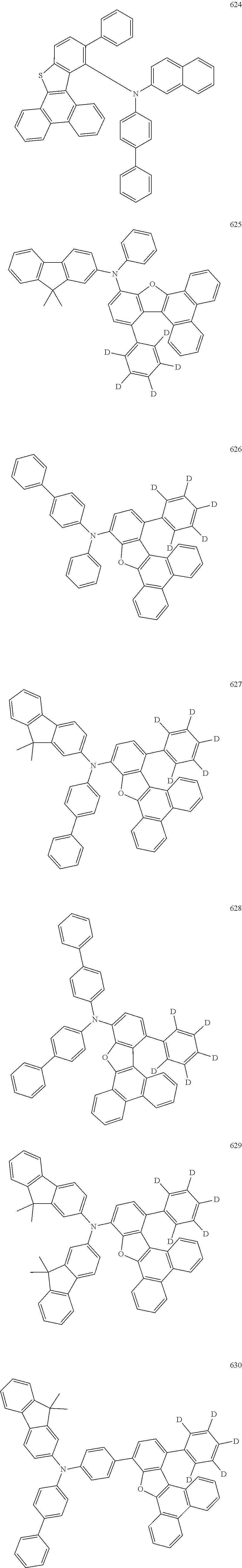

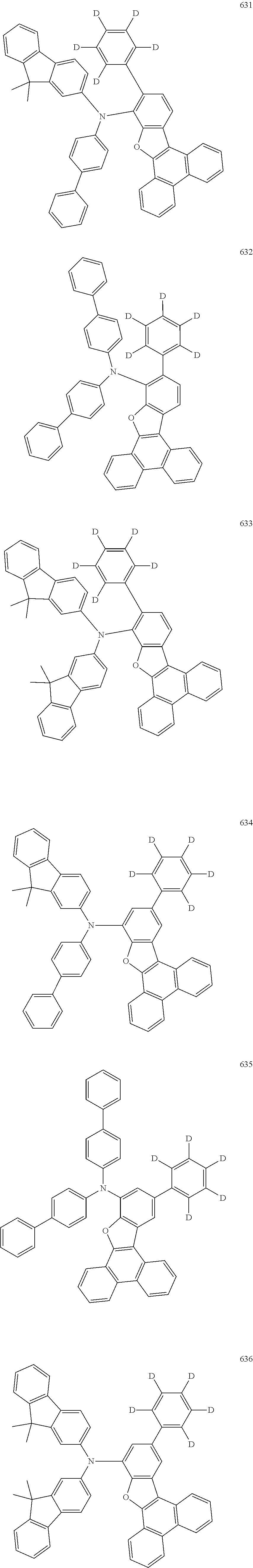

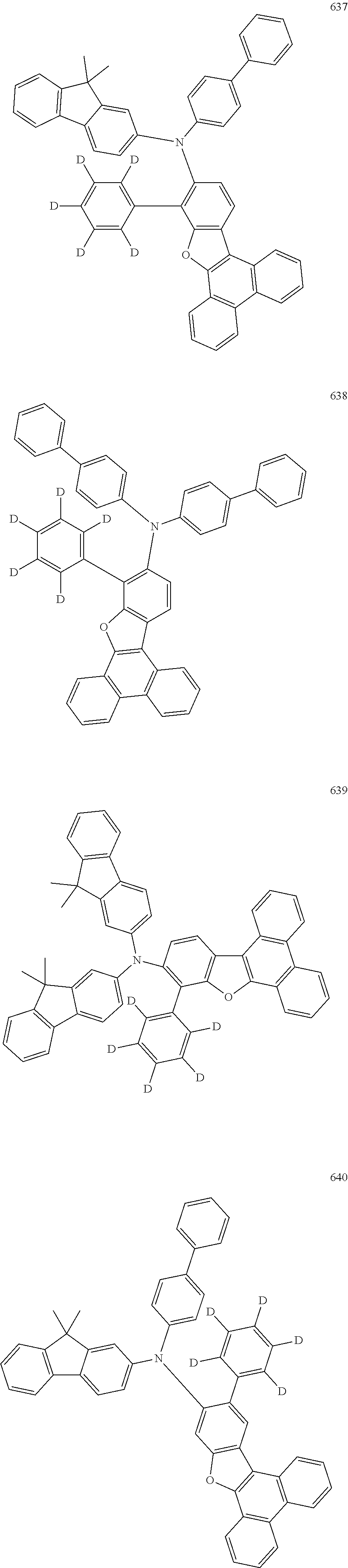 [SMILES: [2H]c1c([2H])c([2H])c(-c2c(N(c3ccc(-c4ccccc4)cc3)c3ccc4c(c3)C(C)(C)c3ccccc3-4)ccc3c2oc2c4ccccc4c4ccccc4c32)c([2H])c1[2H] ; [2H]c1c([2H])c([2H])c(-c2c(N(c3ccc(-c4ccccc4)cc3)c3ccc(-c4ccccc4)cc3)ccc3c2oc2c4ccccc4c4ccccc4c32)c([2H])c1[2H] ; [2H]c1c([2H])c([2H])c(-c2c(N(c3ccc4c(c3)C(C)(C)c3ccccc3-4)c3ccc4c(c3)C(C)(C)c3ccccc3-4)ccc3c2oc2c4ccccc4c4ccccc4c32)c([2H])c1[2H] ; [2H]c1c([2H])c([2H])c(-c2cc3c(cc2N(c2ccc(-c4ccccc4)cc2)c2ccc4c(c2)C(C)(C)c2ccccc2-4)oc2c4ccccc4c4ccccc4c32)c([2H])c1[2H]]

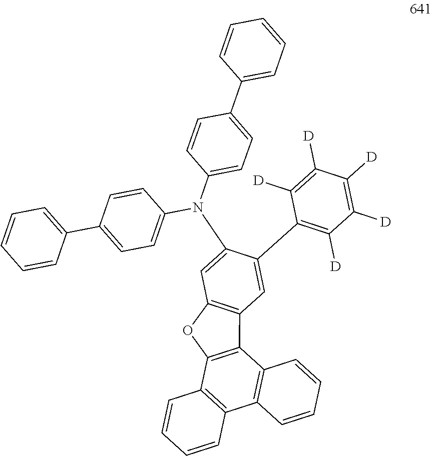 [SMILES: [2H]c1c([2H])c([2H])c(-c2cc3c(cc2N(c2ccc(-c4ccccc4)cc2)c2ccc(-c4ccccc4)cc2)oc2c4ccccc4c4ccccc4c32)c([2H])c1[2H]]

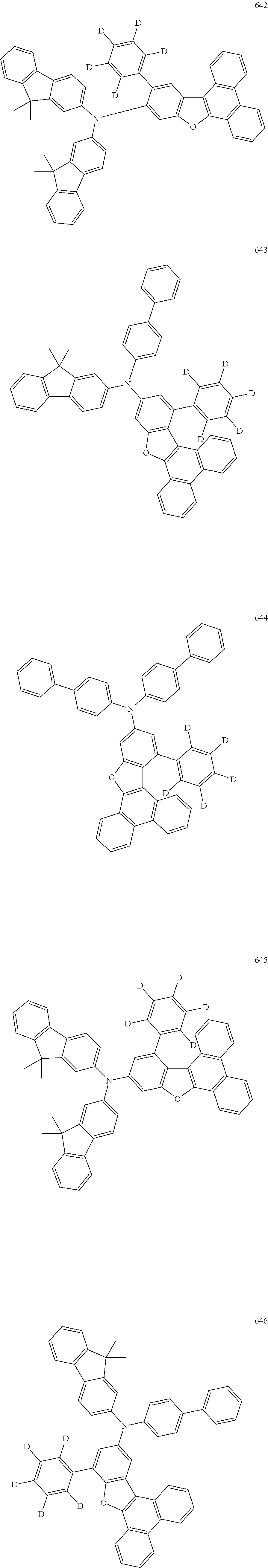

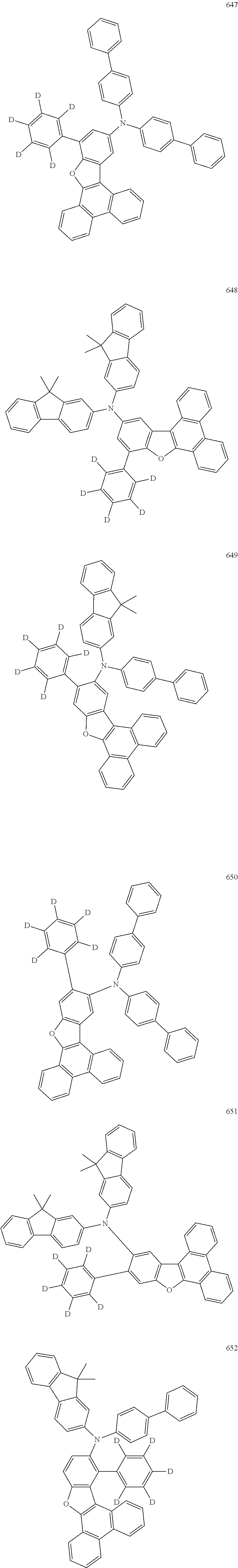

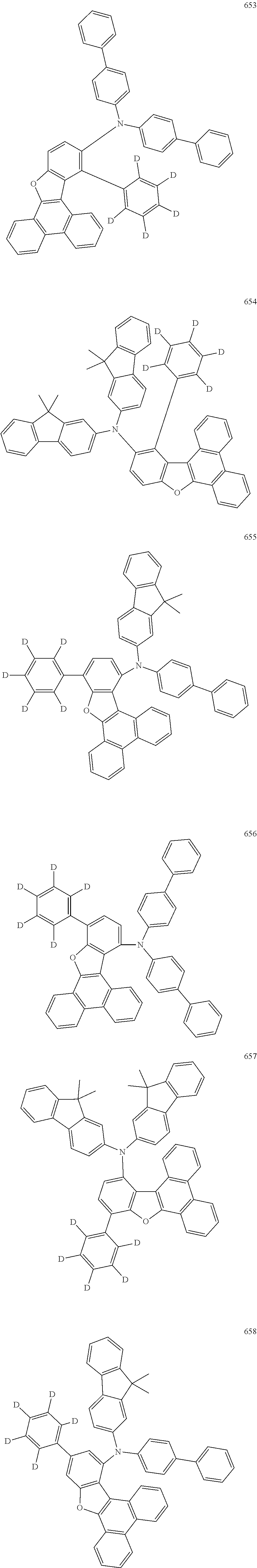

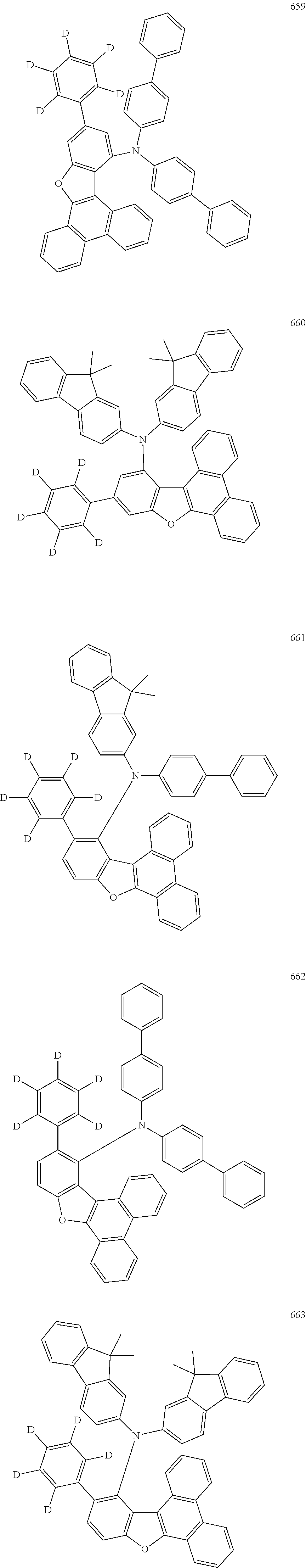

By introducing various substituents into the corresponding structure, the compound of Formula 1 above may be synthesized as a compound having intrinsic properties of the introduced substituent. For example, by introducing into the core structure a substituent mainly used for a material for a hole injection layer, a material for a hole transport layer, a material for an electron-blocking layer, a material for a light-emitting layer, a material for a hole-blocking layer, a material for an electron transport layer, a material for an electron injection layer, and a material for an electron-generating layer, which are used in manufacturing the organic light-emitting device, it is possible to synthesize a material satisfying the conditions required for each organic layer.

In addition, by introducing various substituents into the structure of Formula 1, it is possible to finely control the energy band gap, while improving the properties at the interface between organic materials and diversifying the use of the materials.

The heterocyclic compound may be used as one or more use selected from a material for a hole injection layer, a material for a hole transport layer, a material for an electron-blocking layer, a material for a light-emitting layer, a material for a hole-blocking layer, a material for an electron transport layer, and a material for an electron injection layer, which are used in the organic layer of the organic light-emitting device, and in particular, may be preferably used as a material for a hole transport layer and/or a material for an electron-blocking layer.

By enhancing the hole characteristics in the dibenzofuran skeleton, the heterocyclic compound of the present invention may exhibit excellent performance in the hole transport layer and/or the electron-blocking layer through the control of the band gap and T1 value. Specifically, by widening the band gap and increasing the T1 value, it is possible to exhibit excellent performance in the hole transport layer and/or the electron-blocking layer.

In addition, the present invention relates to an organic light-emitting device comprising: a first electrode; a second electrode provided to face the first electrode; and one or more organic layers provided between the first electrode and the second electrode, wherein the organic layers comprise the heterocyclic compound represented by Formula 1 above.

In one embodiment of the present invention, the first electrode may be an anode, and the second electrode may be a cathode.

In another embodiment, the first electrode may be a cathode, and the second electrode may be an anode.

The organic light-emitting device according to one embodiment of the present invention may comprise on the organic layer one or two more layers selected from the group consisting of a hole injection layer, a hole transport layer, an electron-blocking layer, a light-emitting layer, a hole-blocking layer, an electron transport layer, and an electron injection layer, and may have, but is not limited to, a stacked structure in the order of anode/hole injection layer/hole transport layer/electron-blocking layer/light-emitting layer/hole-blocking layer/electron transport layer/electron injection layer/cathode.

In one embodiment of the present invention, the organic light-emitting device may be a blue organic light-emitting device, and the heterocyclic compound represented by Formula 1 above may be used as a material of the blue organic light-emitting device.

In one embodiment of the present invention, the organic light-emitting device may be a red organic light-emitting device, and the heterocyclic compound represented by Formula 1 may be used as a material of the red organic light-emitting device.

In one embodiment of the present invention, the organic light-emitting device may be a green organic light-emitting device, and the heterocyclic compound represented by Formula 1 above may be used as a material of the green organic light-emitting device.

Specific details of the heterocyclic compound represented by Formula 1 are as described above.

The organic light-emitting device of the present invention may be manufactured by conventional methods and materials for manufacturing an organic light-emitting device, except that one or more organic layers are formed using the aforementioned heterocyclic compound.

In one embodiment of the present invention, in the blue organic light-emitting device, the red organic light-emitting device, and the green organic light-emitting device, the heterocyclic compound represented by Formula 1 above may be used as one or more uses selected from a material for a hole injection layer, a material for a hole transport layer, a material for an electron-blocking layer, a material for a light-emitting layer, a material for a hole-blocking layer, a material for an electron transport layer, and a material for an electron injection layer, and in particular, may be used as a material for a hole transport layer and/or a material for an electron-blocking layer.

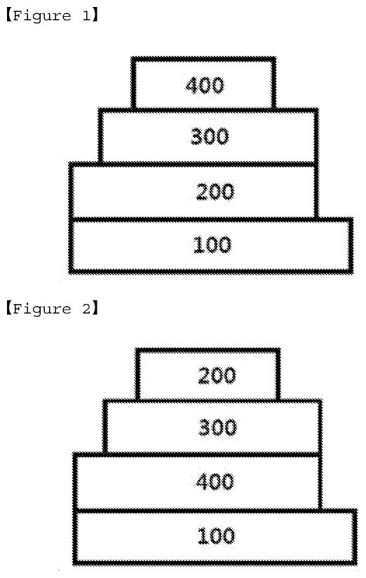

FIGS. 1 to 3 accompanied below illustrate the stacking order of the electrodes and the organic layers of the organic light-emitting device according to one embodiment of the present invention. However, it is not intended that the scope of the present invention be limited by these drawings, and the structure of the organic light-emitting device known in the art may also be applied to the present invention.

According to FIG. 1, there is shown an organic light-emitting device in which an anode 200, an organic layer 300, and a cathode 400 are sequentially stacked on a substrate 100. However, it is not limited to such a structure, and an organic light-emitting device in which a cathode, an organic layer, and an anode are sequentially stacked on a substrate may be implemented, as shown in FIG. 2.

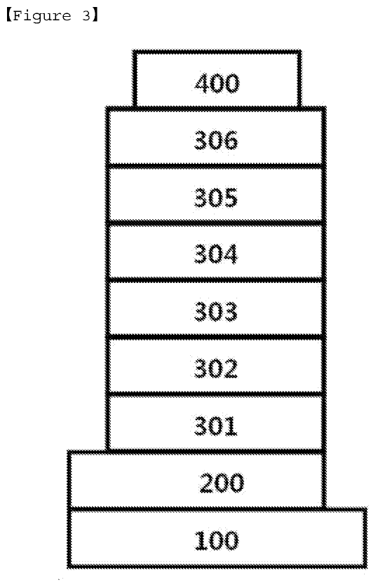

FIG. 3 illustrates a case where the organic layer is composed of multiple layers. The organic light-emitting device according to FIG. 3 comprises a hole injection layer 301, a hole transport layer 302, a light-emitting layer 303, a hole-blocking layer 304, an electron transport layer 305, and an electron injection layer 306. However, the scope of the present invention is not limited by such stacked structures, and the remaining layers except for the light-emitting layer may be omitted, if necessary, and other necessary functional layers such as an electron-blocking layer may be further added.

The heterocyclic compound may be formed into an organic layer by a solution coating method as well as a vacuum deposition method when manufacturing an organic light-emitting device. In this case, the solution coating method refers to, but is not limited to, spin coating, dip coating, inkjet printing, screen printing, spraying, roll coating, and the like.

The organic layer of the organic light-emitting device of the present invention may have a single-layer structure, and may also have a multi-layer structure in which two or more organic layers are stacked. For example, the organic light-emitting device of the present invention may have a structure comprising one or more selected from the group consisting of a hole injection layer, a hole transport layer, an electron-blocking layer, light-emitting layer, a hole-blocking layer, an electron transport layer, an electron injection layer, an electron-generating layer, and the like, as an organic layer. However, the structure of the organic light-emitting device is not limited thereto, and may include a smaller or lager number of organic layers.

In the organic light-emitting device according to one embodiment of the present invention, the materials other than the heterocyclic compound represented by Formula 1 above are exemplified below, but these are for illustrative purposes only and are not intended to limit the scope of the present invention, and may be replaced with materials known in the art.

As the anode material, materials having a relatively large work function may be used, and transparent conductive oxides, metals, conductive polymers, or the like may be used. Specific examples of the anode material include, but are not limited to, metals such as vanadium, chromium, copper, zinc, and gold, or alloys thereof; metal oxides such as zinc oxide, indium oxide, indium tin oxide (ITO), and indium zinc oxide (IZO); a combination of metals and oxides such as ZnO:Al or SnO₂:Sb; conductive polymers such as poly(3-methylthiophene), poly[3,4-(ethylene-1,2-dioxy)thiophene] (PEDOT), polypyrrole, and polyaniline; and the like.

As the cathode material, materials having a relatively low work function may be used, and metals, metal oxides, conductive polymers, or the like may be used. Specific examples of the cathode material include, but are not limited to, metals such as magnesium, calcium, sodium, potassium, titanium, indium, yttrium, lithium, gadolinium, aluminum, silver, tin, and lead, or alloys thereof; multilayer-structured materials such as LiF/Al or LiO₂/Al; and the like.

As the hole injection layer material, a known material for the hole injection layer may be used, for example, phthalocyanine compounds such as copper phthalocyanine, and the like, disclosed in U.S. Pat. No. 4,356,429, or starburst-type amine derivatives such as tris(4-carbazolyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), soluble conductive polymer polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, disclosed in a document [Advanced Material, 6, p. 677 (1994)] may be used.

As a material for the hole transport layer, a pyrazoline derivative, an arylamine-based derivative, a stilbene derivative, a triphenyldiamine derivative, or the like may be used, and a low-molecular weight or high-molecular weight material may be used.

As a material for the electron transport layer, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone and derivatives thereof, diphenyldicyanoethylene and derivatives thereof, diphenoquinone derivatives, 8-hydroxyquinoline and derivatives thereof, and the like may be used, and high-molecular weight materials as well as low-molecular weight materials may be used.

As the electron injection layer material, for example, LiF is typically used in the art, but the present invention is not limited thereto.

As a material for the light-emitting layer, a red, green, or blue light-emitting material may be used, and a mixture of two or more light-emitting materials may be used, if necessary. In this case, it is possible to use by depositing two or more light-emitting materials as separate sources, or it is possible to use by pre-mixing and depositing them as a single source. In addition, as the light-emitting layer material, a fluorescent material may be used, and a phosphorescent material may also be used. As a material for the light-emitting layer, materials that emit light by combining holes and electrons respectively injected from the anode and the cathode may be used alone, and materials in which the host material and the dopant material together participate in light emission may also be used.

When using by mixing hosts of the material for the light-emitting layer, it is possible to use by mixing hosts of the same type, and it is also possible to use by mixing different types of hosts. For example, it is possible to use by selecting any two or more types of n-type host materials or p-type host materials as a host material for the light-emitting layer.

In the phosphorescent material, those known in the art may be used as the phosphorescent dopant material. For example, phosphorescent dopant materials represented by LL′MX′, LL′L″M, LMX′X″, L₂MX′, and L₃M may be used, but the scope of the present invention is not limited by these examples.

The M may be iridium, platinum, osmium, or the like.

The L is an anionic bidentate ligand coordinated to the M by sp² carbon and a heteroatom, and X may function to trap electrons or holes. Non-limiting examples of L include 2-(1-naphthyl)benzoxazole, (2-phenylbenzoxazole), (2-phenylbenzothiazole), (2-phenylbenzothiazole), (7,8-benzoquinoline), (thiophenepyrizine), phenylpyridine, benzothiophenepyrizine, 3-methoxy-2-phenylpyridine, thiophene pyrizine, tolylpyridine, and the like. Non-limiting examples of X′ and X″ include acetylacetonate (acac), hexafluoroacetylacetonate, salicylidene, picolinate, 8-hydroxyquinolinate, and the like.

Specific examples of the phosphorescent dopant are shown below, but are not limited to these examples:

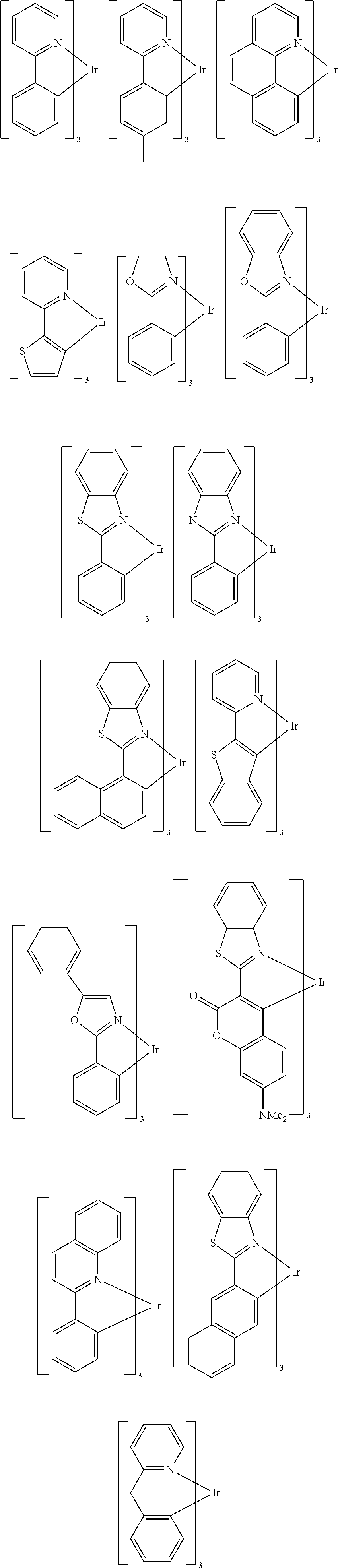

In one embodiment of the present invention, the light-emitting layer includes the heterocyclic compound represented by Formula 1, and may use it together with an iridium-based dopant.

In one embodiment of the present invention, as the iridium-based dopant, the red phosphorescent dopant (piq)₂(Ir) (acac), the green phosphorescent dopant Ir(ppy)₃, and the like may be used.

In one embodiment of the present invention, the content of the dopant may be 1% to 15%, preferably 3% to 10%, more preferably 5% to 10% based on the entire light-emitting layer.

As the material for the electronic-blocking layer, one or more compounds selected from, but not limited to, tris(phenyloyrazole)iridium, 9,9-bis[4-(N,N-bis-biphenyl-4-ylamino)phenyl]-9H-fluorene (BPAPF), bis[4-(p,p-ditolylamino)phenyl]diphenylsilane, NPD (4,4′-bis[N-(1-napthyl)-N-phenylamino]biphenyl), mCP (N,N′-dicarbazolyl-3,5-benzene), and MPMP (bis[4-(N,N-diethylamino)-2-methylphenyl] (4-methylphenyl)methane) may be used.

In addition, the electron-blocking layer may include an inorganic compound. For example, it may include, but is not limited to, at least any one of halide compounds such as LiF, NaF, KF, RbF, CsF, FrF, MgF₂, CaF₂, SrF₂, BaF₂, LiCl, NaCl, KCl, RbCl, CsCl and FrCl and oxides such as Li₂O, Li₂O₂, Na₂O, K₂O, Rb₂O, Rb₂O₂, Cs₂O, Cs₂O₂, LiAlO₂, LiBO₂, LiTaO₃, LiNbO₃, LiWO₄, Li₂CO, NaWO₄, KAlO₂, K₂SiO₃, B₂O₅, Al₂O₃ and SiO₂; or a combination thereof.

As the hole-blocking layer material, an oxadiazole derivative, a triazole derivative, a phenanthroline derivative, BCP, an aluminum complex, and the like may be used without limitation.

In the organic light-emitting device of the present invention, as materials not described above, materials known in the art may be used without limitation.

The organic light-emitting device according to one embodiment of the present invention may be a top emission type, a bottom emission type, or a dual emission type depending on the material to be used.

In addition, the present invention relates to a composition for an organic layer of an organic light-emitting device, comprising the heterocyclic compound represented by Formula 1.

Specific details of the heterocyclic compound represented by Formula 1 are as described above.

The composition for an organic layer may be used as a material for a hole injection layer, a material for a hole transport layer, a material for an electron-blocking layer, a material for a light-emitting layer, a material for a hole-blocking layer, a material for an electron transport layer, and a material for an electron injection layer, and in particular, may be preferably used as a material for a hole transport layer and/or a material for an electron-blocking layer.

The composition for an organic layer may further include materials commonly used in the composition for an organic layer in the art, together with the heterocyclic compound represented by Formula 1. For example, it may further comprise a material, and the like, which are included in order to prepare the heterocyclic compound to be used in the deposition process.

In addition, the present invention relates to a method of manufacturing an organic light-emitting device, comprising the steps of: preparing a substrate; forming a first electrode on the substrate; forming one or more organic layers on the first electrode; and forming a second electrode on the organic layer, wherein the step of forming the organic layers comprises the step of forming one or more organic layers using the hetero compound represented by Formula 1 or the composition for an organic layer of the present invention.

In one embodiment of the present invention, the step of forming the organic layers may be formed by depositing the heterocyclic compound represented by Formula 1 or the composition for an organic layer using a thermal vacuum deposition method.

The organic layer including the composition for an organic layer may further include other materials commonly used in the art, if necessary.

### BEST MODE

The heterocyclic compound represented by Formula 1 according to one embodiment of the present invention may act on the principle similar to that applied to the organic light-emitting device even in an organic electronic device including an organic solar cell, an organic photoreceptor, an organic transistor, and the like.

Hereinafter, preferred examples will be presented to help the understanding of the present invention, but the following examples are provided not to limit the present invention but to facilitate the understanding of the present invention.

### [Preparative Example 1] Preparation of Compound 1

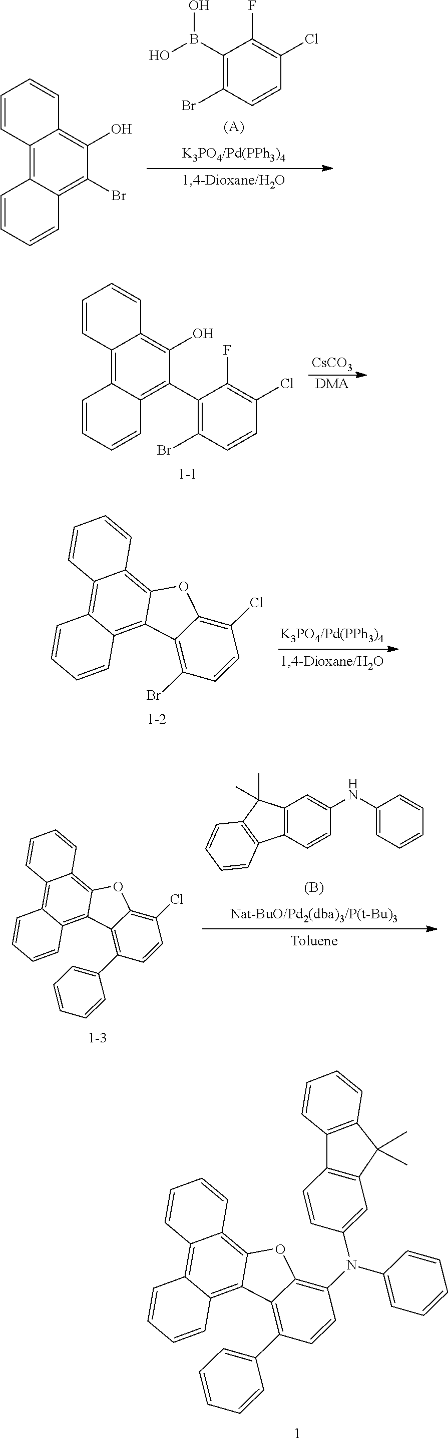

### 1) Preparation of Compound 1-1

Compound 10-bromophenanthren-9-ol (50 g, 0.183 mol, 1 eq), (6-bromo-3-chloro-2-fluorophenyl)boronic acid (A) (50.9 g, 0.201 mol, 1.1 eq), K₃PO₄ (77.7 g, 0.366 mol, 2 eq), and Pd(PPh₃)₄ (10.6 g, 0.0092 mol, 0.05 eq) were placed in 1,4-dioxane (600 ml) and water (150 ml) and stirred at 100° C. for 6 hours. Upon completion of the reaction, it was cooled to room temperature and the reaction was stopped by adding water, and then extraction was performed using MC and water. Thereafter, water was removed with MgSO₄. It was separated by a silica gel column to obtain 51.4 g of Compound 1-1 in a yield of 70%.

### 2) Preparation of Compound 1-2

Compound 1-1 (50 g, 0.124 mol, 1 eq) was placed in DMA (500 ml) and stirred at 140° C. Upon completion of the reaction, it was cooled to room temperature, and then filtered to remove Cs₂CO₃ (80.2 g, 0.246 mol, 2 eq). The filtered solid was washed with water and MeOH, and then dried to obtain 42.6 g of Compound 1-2 in a yield of 90%.

### 3) Preparation of Compound 1-3

Compound 1-2 (40 g, 0.105 mol, 1 eq), phenylboronic acid (14.1 g, 0.116 mol, 1.1 eq), K₃PO₄ (44.6 g, 0.210 mol, 2 eq), and Pd(PPh₃)₄ (6.1 g, 0.0053 mol, 0.05 eq) were placed in 1,4-dioxane (480 ml) and water (120 ml) and stirred at 100° C. for 6 hours. Upon completion of the reaction, it was cooled to room temperature and the reaction was stopped by adding water, and then extraction was performed using MC and water. Thereafter, water was removed with MgSO₄. It was separated by a silica gel column to obtain 33.8 g of Compound 1-3 in a yield of 85%.

### 4) Preparation of Compound 1

9,9-dimethyl-N-phenyl-9H-fluoren-2-amine (B) (10 g, 0.035 mol, 1 eq), Compound 1-3 (14 g, 0.037 mol, 1.05 eq), NaOt-Bu (6.7 g, 0.070 mol, 2 eq), Pd₂(dba)₃ (1.6 g, 0.0018 mol, 0.05 eq), and P(t-Bu)₃ (0.7 g, 0.0035 mol, 0.1 eq) were placed in toluene (100 ml) and stirred at 100° C. for 3 hours. After the reaction was stopped by adding water, extraction was performed using MC and water. Thereafter, water was removed with MgSO₄. It was separated by a silica gel column to obtain 15.4 g of Compound 1 in a yield of 76%.

The compound was synthesized in the same manner as in Preparative Example 1 above, except that Intermediate A of Table 1 below instead of (6-bromo-3-chloro-2-fluorophenyl)boronic acid (A) and Intermediate B of Table 1 below instead of 9,9-dimethyl-N-phenyl-9H-fluorene-2-amine (B) were used.

### [Preparative Example 2] Preparation of Compound 49

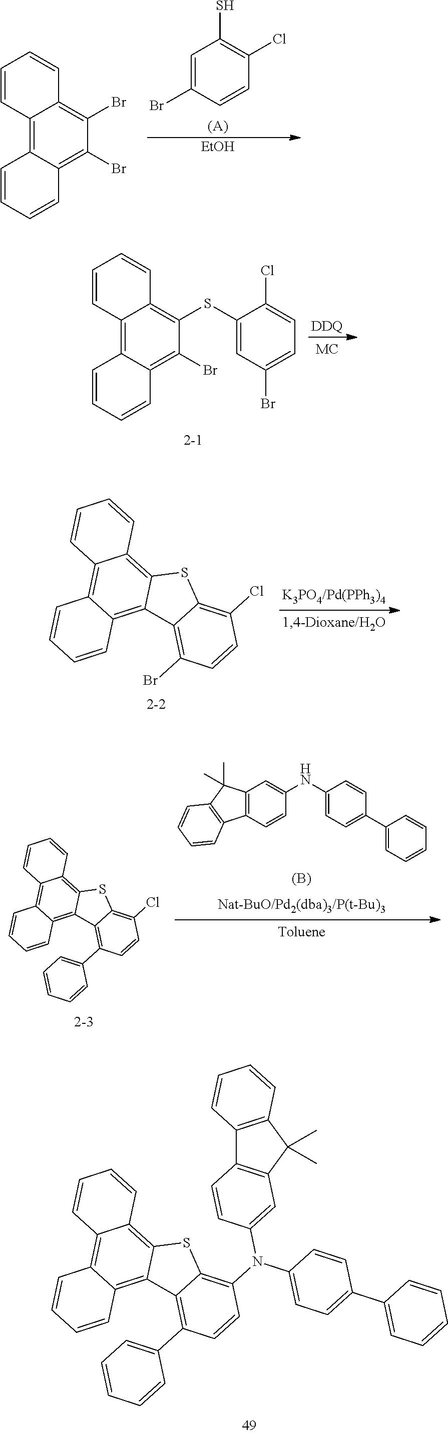

### 1) Preparation of Compound 2-1

5-bromo-2-chlorobenzenethiol (A) (30 g, 0.134 mol, 1.0 eq), 9,10-dibromophenanthrene (54.1 g, 0.161 mol, 1.2 eq), and NaOH (10.7 g, 0.268 mol, 2.5 eq) were placed in EtOH (300 ml) and stirred under reflux for 4 hours. Upon completion of the reaction, it was cooled to room temperature and the reaction was stopped by adding water, and then extraction was performed using MC and water. Thereafter, water was removed with MgSO₄. It was separated by a silica gel column to obtain 51.3 g of Compound 2-1 in a yield of 80%.

### 2) Preparation of Compound 2-2

Compound 2-3 (50 g, 0.104 mol, 1 eq) and 2,3-dichloro-5,6-dicyano-1,4-benzoquinone (DDQ) (47.2 g, 0.208 mol, 2 eq) were placed in dichloro methane (MC) (500 ml) and stirred at room temperature for 24 hours. Upon completion of the reaction, the reaction was stopped by adding water, and then extraction was performed using MC and water. Thereafter, water was removed with MgSO₄. It was separated by a silica gel column to obtain 35.1 g of Compound 2-2 in a yield of 80%.

### 3) Preparation of Compound 2-3

Compound 2-3 was obtained in the same manner as in the synthesis method of Compound 1-3 in Preparative Example 1 above.

### 4) Preparation of Compound 49

Compound 49 was obtained in the same manner as in the synthesis method of Compound 1 in Preparative Example 1 above.

The compound was synthesized in the same manner as in Preparative Example 2 above, except that Intermediate A of Table 1 below instead of 5-bromo-2-chlorobenzenethiol (A) and Intermediate B of Table 1 below instead of 9,9-dimethyl-N-phenyl-9H-fluorene-2-amine (B) were used.

TABLE 1

| Com- | | | | |
|---|---|---|---|---|
| pound | | | | |
| No. | Intermediate A | Intermediate B | Intermediate C | Yield |
| 17 | 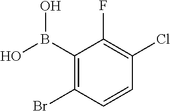 [SMILES: OB(O)c1c(Br)ccc(Cl)c1F] | 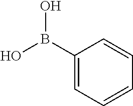 | 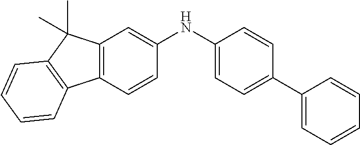 [SMILES: CC1(C)c2ccccc2-c2ccc(Nc3ccc(-c4ccccc4)cc3)cc21] | 75% |
| 18 | 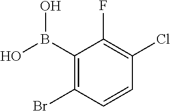 [SMILES: OB(O)c1c(Br)ccc(Cl)c1F] | 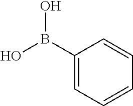 | 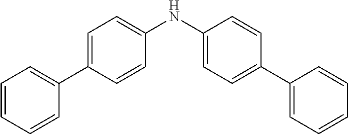 [SMILES: c1ccc(-c2ccc(Nc3ccc(-c4ccccc4)cc3)cc2)cc1] | 78% |
| 21 | 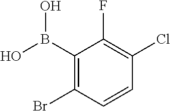 [SMILES: OB(O)c1c(Br)ccc(Cl)c1F] | 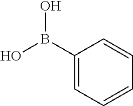 | 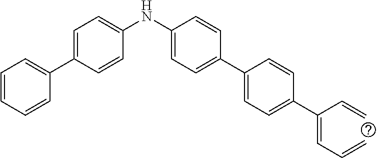 [SMILES: C=C/C=C(\C=C)c1ccc(-c2ccc(Nc3ccc(-c4ccccc4)cc3)cc2)cc1] | 80% |
| 22 | 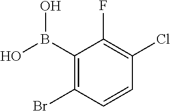 [SMILES: OB(O)c1c(Br)ccc(Cl)c1F] | 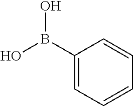 | 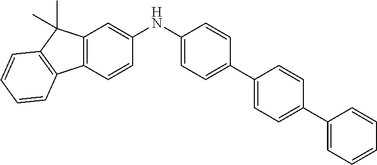 [SMILES: CC1(C)c2ccccc2-c2ccc(Nc3ccc(-c4ccc(-c5ccccc5)cc4)cc3)cc21] | 75% |
| 23 | 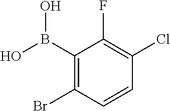 [SMILES: OB(O)c1c(Br)ccc(Cl)c1F] | 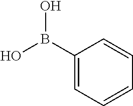 | 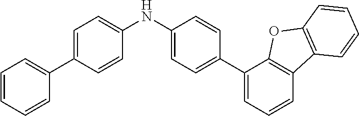 [SMILES: c1ccc(-c2ccc(Nc3ccc(-c4cccc5c4oc4ccccc45)cc3)cc2)cc1] | 70% |
| 24 | 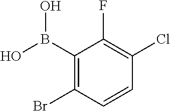 [SMILES: OB(O)c1c(Br)ccc(Cl)c1F] | 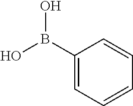 | 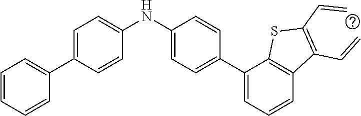 [SMILES: C=Cc1sc2c(-c3ccc(Nc4ccc(-c5ccccc5)cc4)cc3)cccc2c1C=C] | 68% |
| 25 | 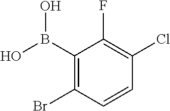 [SMILES: OB(O)c1c(Br)ccc(Cl)c1F] | 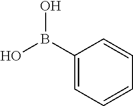 | 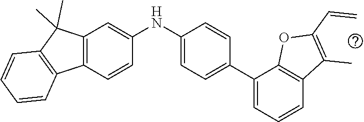 [SMILES: C=Cc1oc2c(-c3ccc(Nc4ccc5c(c4)C(C)(C)c4ccccc4-5)cc3)cccc2c1C] | 65% |
| 26 | 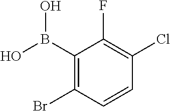 [SMILES: OB(O)c1c(Br)ccc(Cl)c1F] | 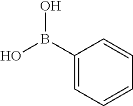 | 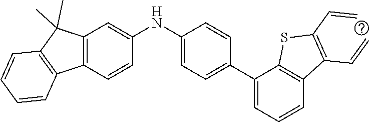 [SMILES: C=Cc1sc2c(-c3ccc(Nc4ccc5c(c4)C(C)(C)c4ccccc4-5)cc3)cccc2c1C=C] | 63% |
| 29 | 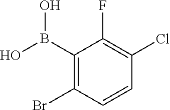 [SMILES: OB(O)c1c(Br)ccc(Cl)c1F] | 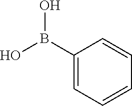 | 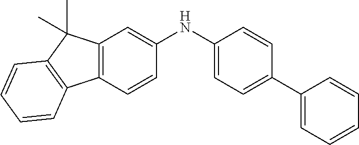 [SMILES: CC1(C)c2ccccc2-c2ccc(Nc3ccc(-c4ccccc4)cc3)cc21] | 85% |
| 33 | 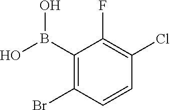 [SMILES: OB(O)c1c(Br)ccc(Cl)c1F] | 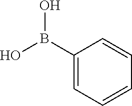 | 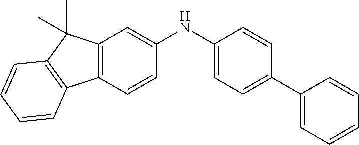 [SMILES: CC1(C)c2ccccc2-c2ccc(Nc3ccc(-c4ccccc4)cc3)cc21] | 60% |
| 34 | 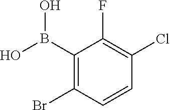 [SMILES: OB(O)c1c(Br)ccc(Cl)c1F] | 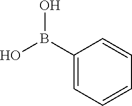 | 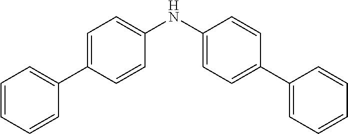 [SMILES: c1ccc(-c2ccc(Nc3ccc(-c4ccccc4)cc3)cc2)cc1] | 63% |
| 35 | 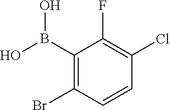 [SMILES: OB(O)c1c(Br)ccc(Cl)c1F] | 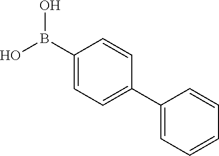 [SMILES: OB(O)c1ccc(-c2ccccc2)cc1] | 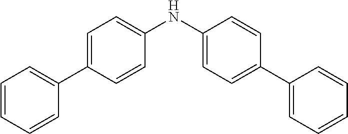 [SMILES: c1ccc(-c2ccc(Nc3ccc(-c4ccccc4)cc3)cc2)cc1] | 65% |
| 36 | 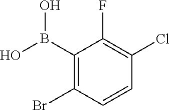 [SMILES: OB(O)c1c(Br)ccc(Cl)c1F] | 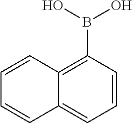 [SMILES: OB(O)c1cccc2ccccc12] | 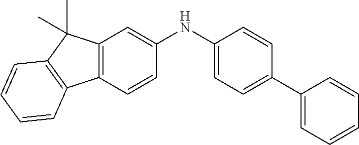 [SMILES: CC1(C)c2ccccc2-c2ccc(Nc3ccc(-c4ccccc4)cc3)cc21] | 70% |
| 42 | 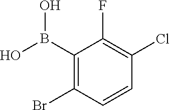 [SMILES: OB(O)c1c(Br)ccc(Cl)c1F] | 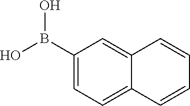 [SMILES: OB(O)c1ccc2ccccc2c1] | 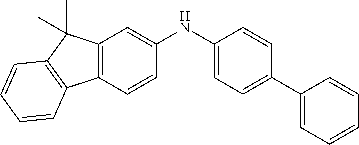 [SMILES: CC1(C)c2ccccc2-c2ccc(Nc3ccc(-c4ccccc4)cc3)cc21] | 63% |
| 45 | 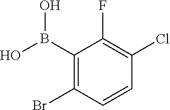 [SMILES: OB(O)c1c(Br)ccc(Cl)c1F] | 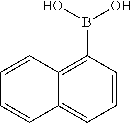 [SMILES: OB(O)c1cccc2ccccc12] | 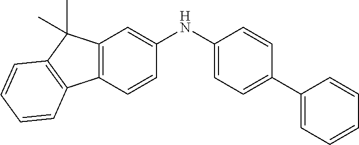 [SMILES: CC1(C)c2ccccc2-c2ccc(Nc3ccc(-c4ccccc4)cc3)cc21] | 68% |
| 69 | 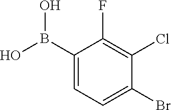 [SMILES: OB(O)c1ccc(Br)c(Cl)c1F] | 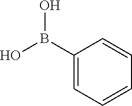 | 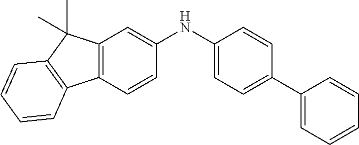 [SMILES: CC1(C)c2ccccc2-c2ccc(Nc3ccc(-c4ccccc4)cc3)cc21] | 63% |
| 70 | 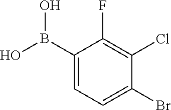 [SMILES: OB(O)c1ccc(Br)c(Cl)c1F] | 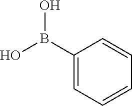 | 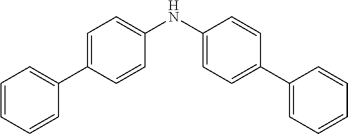 [SMILES: c1ccc(-c2ccc(Nc3ccc(-c4ccccc4)cc3)cc2)cc1] | 72% |
| 121 | 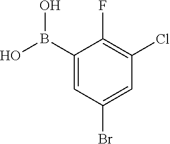 [SMILES: OB(O)c1cc(Br)cc(Cl)c1F] | 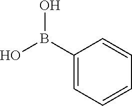 | 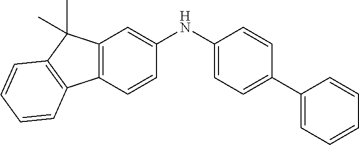 [SMILES: CC1(C)c2ccccc2-c2ccc(Nc3ccc(-c4ccccc4)cc3)cc21] | 75% |
| 122 | 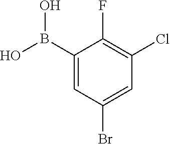 [SMILES: OB(O)c1cc(Br)cc(Cl)c1F] | 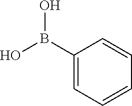 | 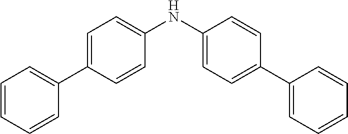 [SMILES: c1ccc(-c2ccc(Nc3ccc(-c4ccccc4)cc3)cc2)cc1] | 80% |
| 153 | 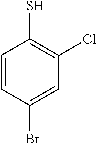 | 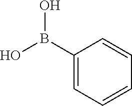 | 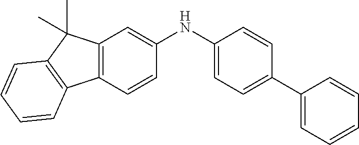 [SMILES: CC1(C)c2ccccc2-c2ccc(Nc3ccc(-c4ccccc4)cc3)cc21] | 75% |
| 173 | 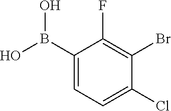 [SMILES: OB(O)c1ccc(Cl)c(Br)c1F] | 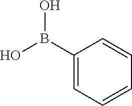 | 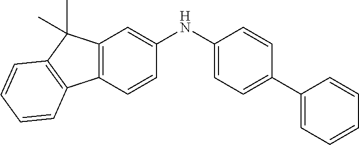 [SMILES: CC1(C)c2ccccc2-c2ccc(Nc3ccc(-c4ccccc4)cc3)cc21] | 68% |
| 225 | 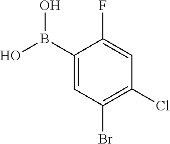 [SMILES: OB(O)c1cc(Br)c(Cl)cc1F] | 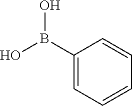 | 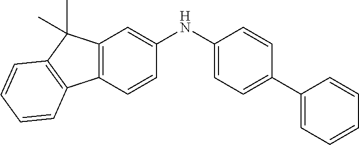 [SMILES: CC1(C)c2ccccc2-c2ccc(Nc3ccc(-c4ccccc4)cc3)cc21] | 51% |
| 277 | 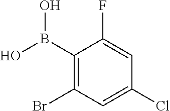 [SMILES: OB(O)c1c(F)cc(Cl)cc1Br] | 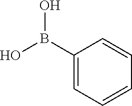 | 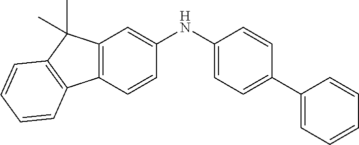 [SMILES: CC1(C)c2ccccc2-c2ccc(Nc3ccc(-c4ccccc4)cc3)cc21] | 70% |
| 309 | 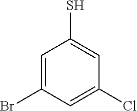 | 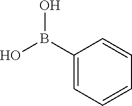 | 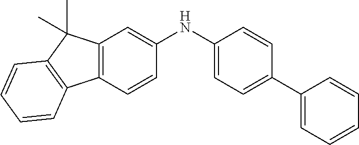 [SMILES: CC1(C)c2ccccc2-c2ccc(Nc3ccc(-c4ccccc4)cc3)cc21] | 73% |
| 329 | 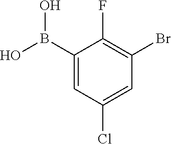 [SMILES: OB(O)c1cc(Cl)cc(Br)c1F] | 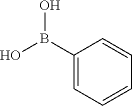 | 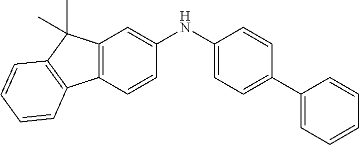 [SMILES: CC1(C)c2ccccc2-c2ccc(Nc3ccc(-c4ccccc4)cc3)cc21] | 70% |
| 361 | 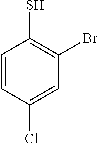 | 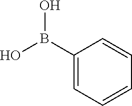 | 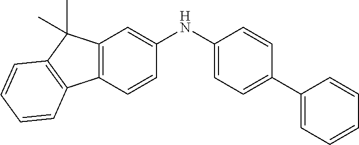 [SMILES: CC1(C)c2ccccc2-c2ccc(Nc3ccc(-c4ccccc4)cc3)cc21] | 73% |
| 381 | 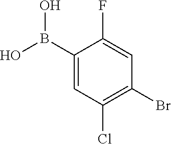 [SMILES: OB(O)c1cc(Cl)c(Br)cc1F] | 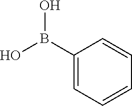 | 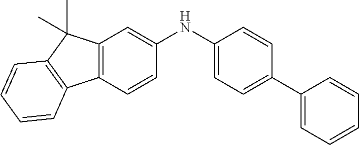 [SMILES: CC1(C)c2ccccc2-c2ccc(Nc3ccc(-c4ccccc4)cc3)cc21] | 53% |
| 433 | 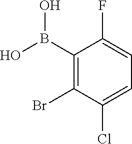 [SMILES: OB(O)c1c(F)ccc(Cl)c1Br] | 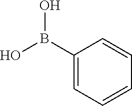 | 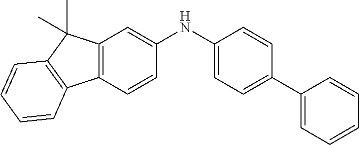 [SMILES: CC1(C)c2ccccc2-c2ccc(Nc3ccc(-c4ccccc4)cc3)cc21] | 55% |
| 485 | 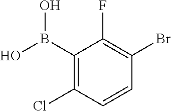 [SMILES: OB(O)c1c(Cl)ccc(Br)c1F] | 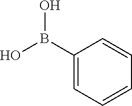 | 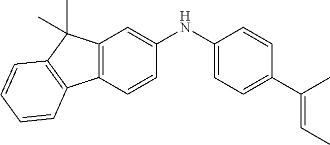 [SMILES: C/C=C(\C)c1ccc(Nc2ccc3c(c2)C(C)(C)c2ccccc2-3)cc1] | 55% |
| 537 | 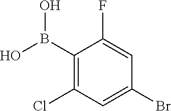 [SMILES: OB(O)c1c(F)cc(Br)cc1Cl] | 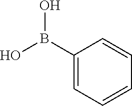 | 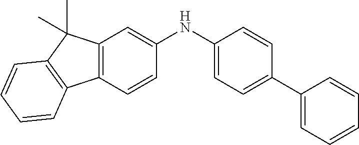 [SMILES: CC1(C)c2ccccc2-c2ccc(Nc3ccc(-c4ccccc4)cc3)cc21] | 50% |
| 589 | 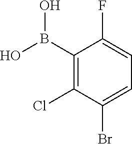 [SMILES: OB(O)c1c(F)ccc(Br)c1Cl] | 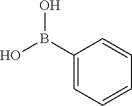 | 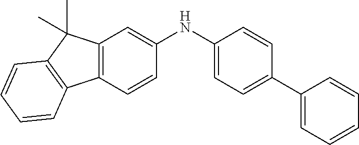 [SMILES: CC1(C)c2ccccc2-c2ccc(Nc3ccc(-c4ccccc4)cc3)cc21] | 45% |
| 627 | 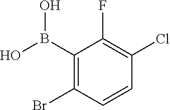 [SMILES: OB(O)c1c(Br)ccc(Cl)c1F] | 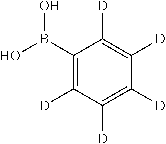 [SMILES: [2H]c1c([2H])c([2H])c(B(O)O)c([2H])c1[2H]] | 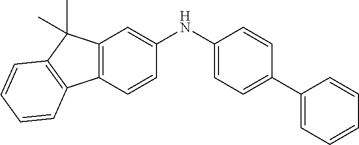 [SMILES: CC1(C)c2ccccc2-c2ccc(Nc3ccc(-c4ccccc4)cc3)cc21] | 75% |
| 655 | 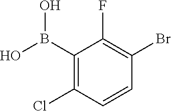 [SMILES: OB(O)c1c(Cl)ccc(Br)c1F] | 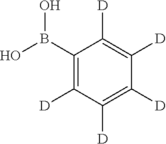 [SMILES: [2H]c1c([2H])c([2H])c(B(O)O)c([2H])c1[2H]] | 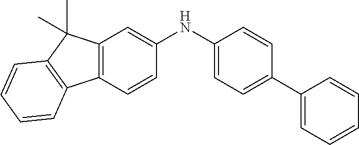 [SMILES: CC1(C)c2ccccc2-c2ccc(Nc3ccc(-c4ccccc4)cc3)cc21] | 55% |

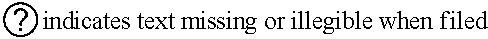

Compounds were prepared in the same manner as in the above preparative examples and the synthesis confirmation results are shown in Tables 2 and 3. Table 2 shows the measured values of ¹H NMR (CDCl₃, 300 Mz) and Table 3 shows the measured values of field desorption mass spectrometry (FD-MS).

TABLE 2

| Compound | ¹H NMR(CDCl₃, 300 Mz) |
|---|---|
| 1 | δ = 9.08(1H, d), 8.98(1H, d), 8.17(1H, d), 8.11(1H, d), 7.90- |
| | 7.78(5H, m), 7.70-7.55(5H, m), 7.46-7.00(13H, m) 1.69(6H, s) |
| 17 | δ = 9.08(1H, d), 8.98(1H, d), 8.17(1H, d), 8.11(1H, d), 7.90- |
| | 7.86(2H, m), 7.79-7.50(5H, m), 7.70-7.55(5H, m), 7.49-7.38(6H, m), |
| | 7.33-7.16(9H, m), 1.69(6H, s) |
| 18 | δ = 9.08(1H, d), 8.98(1H, d), 8.17(1H, d), 8.11(1H, d), 7.79- |
| | 7.62(10H, m), 7.55-7.41(13H, m), 1.69(6H, s) |
| 21 | δ = 9.08(1H, d), 8.98(1H, d), 8.17(1H, d), 8.11(1H, d), 7.75- |
| | 7.62(11H, m), 7.55-7.41(13H, m), 7.37-7.16(9H, m) |
| 22 | δ = 9.08(1H, d), 8.98(1H, d), 8.17(1H, d), 8.11(1H, d), 7.90- |
| | 7.86(2H, m), 7.79-7.70(6H, m), 7.68-7.55(6H, m), 7.49-7.41(6H, m), |
| | 7.37-7.16(11H, m), 1.69(6H, s) |
| 23 | δ = 9.08(1H, d), 8.98(1H, d), 8.17(1H, d), 8.11(1H, d), 8.08- |
| | 7.98(3H, m), 7.79-7.70(6H, m), 7.68-7.51(9H, m), 7.49-7.41(6H, m), |
| | 7.39-7.16(7H, m) |
| 24 | δ = 9.08(1H, d), 8.98(1H, d), 8.55(1H, d), 8.45(1H, d), 8.32(1H, d) |
| | 8.17(1H, d), 8.11(1H, d), 7.93(1H, d), 7.79-7.70(7H, m), 7.68- |
| | 7.55(8H, m), 7.49-7.41(7H, m), 7.37-7.16(5H, m) |
| 25 | δ = 9.08(1H, d), 8.98(1H, d), 8.17(1H, d), 8.11(1H, d), 8.08(1H, |
| | d), 8.02(1H, d), 7.98-7.86(3H, m), 7.79-7.78(3H, m), 7.70-7.62(4H, |
| | m), 7.46-7.41(8H, m), 7.39-7.28(7H, m), 7.16(2H, d), 1.69(6H, s) |
| 26 | δ = 9.08(1H, d), 8.98(1H, d), 8.55(1H, d), 8.45(1H, d), 8.32(1H, |
| | d), 8.17(1H, d), 8.11(1H, d), 7.93-7.86(3H, m), 7.79-7.78(3H, m), |
| | 7.70-7.62(5H, m), 7.56-7.46(7H, m), 7.41-7.28(6H, m), 7.16(2H, d), |
| | 1.69(6H, s) |
| 29 | δ = 9.08(1H, d), 8.98(1H, d), 8.17(1H, d), 8.11(1H, d), 8.07(1H, |
| | d), 8.01(1H, d), 7.90(1H, d), 7.86(1H, d), 7.79-7.70(5H, m), 7.68- |
| | 7.55(8H, m), 7.49-7.41(6H, m), 7.37-7.28(7H, m), 7.16(1H, d), |
| | 1.69(6H, s) |
| 33 | δ = 9.08(1H, d), 8.98-8.96(2H, d), 8.27(1H, d), 8.17(1H, d), |
| | 8.11(1H, d), 8.07(1H, d), 8.01(1H, d), 7.90(1H, d), 7.86(1H, d), |
| | 7.79-7.70(5H, m), 7.68-7.54(8H, m), 7.49-7.41(7H, m), 7.38-7.28(6H, |
| | m), 7.16(1H, m), 1.69(6H, s) |
| 34 | δ = 9.08(1H, d), 8.98(1H, d), 8.17(1H, d), 8.11(1H, d), 8.07(1H, |
| | d), 8.01(1H, d), 7.79-7.70(7H, m), 7.68-7.62(3H, m), 7.55-7.46(12H, |
| | m), 7.41-7.37(9H, m), 7.25(4H, s) |
| 35 | δ = 9.08(1H, d), 8.98(1H, d), 8.17(1H, d), 8.11(1H, d), 8.07(1H, |
| | d), 8.01(1H, d), 7.75-7.62(10H, m), 7.55-7.46(12H, m), 7.41- |
| | 7.37(9H, m), 7.25(4H, s) |
| 36 | δ = 9.08(1H, d), 8.98(1H, d), 8.95(1H, d), 8.50(1H, d), 8.20- |
| | 8.09(4H, m), 7.90-7.78(3H, m), 7.77-7.70(6H, m), 7.68-7.52(5H, m), |
| | 7.49-7.28(9H, m), 7.16(2H, d), 1.69(6H, s) |
| 42 | δ = 9.08(1H, d), 8.98(1H, d), 8.17(1H, d), 8.11(1H, d), 8.09(1H, |
| | d), 8.06(1H, d), 7.99(1H, d), 7.90(1H, d), 7.86(1H, d), 7.78- |
| | 7.62(9H, m), 7.55-7.41(7H, m), 7.38-7.28(6H, m), 7.16(2H, d), |
| | 1.69(6H, s) |
| 45 | δ = 9.08(1H, d), 8.98(1H, d), 8.95(1H, d), 8.50(1H, d), 8.20- |
| | 8.01(6H, m), 7.90-7.86(2H, m), 7.77-7.62(7H, m), 7.55-7.41(9H, m), |
| | 7.39-7.28(8H, m), 7.16(1H, d), 1.69(6H, s) |
| 49 | δ = 9.08(1H, d), 8.98(1H, d), 8.17(1H, d), 8.11(1H, d), 7.90- |
| | 7.86(3H, d), 7.79-7.60(9H, m), 7.55-7.41(9H, m), 7.38-7.28(5H, m), |
| | 7.16(1H, d), 1.69(6H, s) |
| 69 | δ = 9.08(1H, d), 8.98(1H, d), 8.17(1H, d), 8.11(1H, d), 7.90- |
| | 7.86(2H, d), 7.78-7.62(8H, m), 7.55-7.41(9H, m), 7.38-7.28(5H, m), |
| | 7.16(1H, d), 7.08(2H, d), 1.69(6H, s) |
| 70 | δ = 9.08(1H, d), 8.98(1H, d), 8.17(1H, d), 8.11(1H, d), 7.78- |
| | 7.62(10H, d), 7.55-7.37(17H, m), 7.08(2H, d) |
| 121 | δ = 9.08(1H, d), 8.98(1H, d), 8.26(1H, s), 8.17(1H, d), 8.11(1H, |
| | d), 7.90-7.86(2H, d), 7.75-7.62(8H, m), 7.55-7.41(10H, m), 7.38- |
| | 7.28(5H, m), 7.16(1H, d), 1.69(6H, s) |
| 122 | δ = 9.08(1H, d), 8.98(1H, d), 8.26(1H, s), 8.17(1H, d), 8.11(1H, |
| | d), 7.75-7.62(10H, m), 7.55-7.37(18H, m) |
| 153 | δ = 9.08(1H, d), 8.98(1H, d), 8.17(1H, d), 8.11(1H, d), 7.90- |
| | 7.82(3H, d), 7.78-7.62(9H, m), 7.55-7.41(9H, m), 7.38-7.28(5H, m), |
| | 7.16(1H, d), 1.69(6H, s) |
| 173 | δ = 9.08(1H, d), 8.98(1H, d), 8.17(1H, d), 8.11(1H, d), 7.90- |
| | 7.86(3H, d), 7.75-7.62(6H, m), 7.55-7.41(11H, m), 7.38-7.28(5H, |
| | m), 7.16(1H, d), 7.10(1H, d), 1.69(6H, s) |
| 225 | δ = 9.08(1H, d), 8.98(1H, d), 8.22(1H, s), 8.17(1H, d), 8.11(1H, |
| | d), 7.90(1H, d), 7.86(1H, d), 7.79(1H, s), 7.75-7.62(6H, m), 7.55- |
| | 7.41(8H, m), 7.43-7.28(6H, m), 7.16(1H, d), 7.08(2H, d), 1.69(6H, |
| | s) |
| 277 | δ = 9.08(1H, d), 8.98(1H, d), 8.20-8.11(4H, m), 7.90(1H, d), |
| | 7.86(1H, d), 7.79-7.62(8H, m), 7.55-7.41(9H, m), 7.38-7.28(5H, m), |
| | 7.16(1H, d), 1.69(6H, s) |
| 309 | δ = 9.08(1H, d), 8.98(1H, d), 8.17(1H, d), 8.11(1H, d), 7.90- |
| | 7.84(3H, m), 7.79-7.62(9H, m), 7.55-7.41(9H, m), 7.38-7.28(5H, m), |
| | 7.16(1H, d), 1.69(6H, s) |
| 329 | δ = 9.08(1H, d), 8.98(1H, d), 8.32(1H, s), 8.26(1H, s), 8.17(1H, |
| | d), 8.11(1H, d), 7.90(1H, d), 7.86(1H, d), 7.75-7.62(6H, m), 7.55- |
| | 7.41(11H, m), 7.38-7.28(5H, m), 7.16(1H, d), 1.69(6H, s) |
| 361 | δ = 9.08(1H, d), 8.98(1H, d), 8.17(1H, d), 8.11(1H, d), 8.05(1H, |
| | s), 7.90-7.82(3H, m), 7.75-7.62(6H, m), 7.55-7.41(11H, m), 7.38- |
| | 7.28(5H, m), 7.16(1H, d), 1.69(6H, s) |
| 381 | δ = 9.08(1H, d), 8.98(1H, d), 8.41(1H, s), 8.17(1H, d), 8.11(1H, |
| | d), 7.90(1H, d), 7.86(1H, d), 7.75-7.62(7H, m), 7.55-7.41(9H, m), |
| | 7.38-7.28(5H, m), 7.16(1H, d), 7.08(2H, d), 1.69(6H, s) |
| 433 | δ = 9.08(1H, d), 8.98(1H, d), 8.17(1H, d), 8.11(1H, d), 7.90(1H, |
| | d), 7.86(1H, d), 7.75-7.62(7H, m), 7.55-7.41(11H, m), 7.38-7.28(5H, |
| | m), 7.16(2H, d), 1.69(6H, s) |
| 485 | δ = 9.08(1H, d), 8.98(1H, d), 8.17(1H, d), 8.11(1H, d), 7.90- |
| | 7.84(3H, m), 7.75-7.62(6H, m), 7.55-7.41(11H, m), 7.38-7.28(5H, |
| | m), 7.16(1H, d), 7.10(1H, d), 1.69(6H, s) |
| 537 | δ = 9.08(1H, d), 8.98(1H, d), 8.20-8. 11(3H, m), 7.90(1H, d), |
| | 7.86(1H, d), 7.75-7.62(8H, m), 7.55-7.41(10H, m), 7.38-7.28(5H, |
| | m), 7.16(1H, d), 1.69(6H, s) |
| 589 | δ = 9.08(1H, d), 8.98(1H, d), 8.17(1H, d), 8.11(1H, d), 7.90- |
| | 7.84(3H, m), 7.75-7.62(6H, m), 7.55-7.41(10H, m), 7.38-7.28(5H, |
| | m), 7.16(1H, d), 7.08(2H, d), 1.69(6H, s) |
| 627 | δ = 9.08(1H, d), 8.98(1H, d), 8.17(1H, d), 8.11(1H, d), 7.90(1H, |
| | d), 7.86(1H, d), 7.78-7.62(7H, m), 7.55-7.41(6H, m), 7.38-7.28(5H, |
| | m), 7.16(2H, d), 1.69(6H, s) |
| 655 | δ = 9.08(1H, d), 8.98(1H, d), 8.17(1H, d), 8.11(1H, d), 7.90- |
| | 7.84(3H, m), 7.75-7.62(6H, m), 7.55-7.41(6H, m), 7.38-7.28(5H, m), |
| | 7.16(1H, d), 7.10(1H, d), 1.69(6H, s) |

TABLE 3

| Compound | FD-MS | Compound | FD-MS |
|---|---|---|---|
| 1 | m/z = 627.26 | 17 | m/z = 703.29 |
| | (C47H33NO = 627.79) | | (C53H37NO = 703.88) |
| 18 | m/z = 663.26 | 21 | m/z = 739.29 |
| | (C50H33NO = 663.82) | | (C56H37NO = 739.92) |
| 22 | m/z = 779.32 | 23 | m/z = 753.27 |
| | (C59H41NO = 779.98) | | (C56H35NO2 = 753.90) |
| 24 | m/z = 769.24 | 25 | m/z = 793.30 |
| | (C56H35NOS = 769.96) | | (C59H39NO2 = 793. 97) |
| 26 | m/z = 809.28 | 29 | m/z = 779.32 |
| | (C59H39NOS = 810.03) | | (C59H41NO = 779.98) |
| 33 | m/z = 829.33 | 34 | m/z = 815.32 |
| | (C63H43NO = 830.04) | | (C62H41NO = 816.02) |
| 35 | m/z = 815.32 | 36 | m/z = 753.30 |
| | (C62H41NO = 816.02) | | (C57H39NO = 753.94) |
| 42 | m/z = 753.30 | 45 | m/z = 829.33 |
| | (C57H39NO = 753.94) | | (C63H43NO = 830.04) |
| 49 | m/z = 719.26 | 69 | m/z = 703.29 |
| | (C53H37NS = 719.95) | | (C53H37NO = 703.88) |
| 70 | m/z = 663.26 | 121 | m/z = 703.29 |
| | (C50H33NO = 663.82) | | (C53H37NO = 703.88) |
| 122 | m/z = 663.26 | 153 | m/z = 719.26 |
| | (C50H33NO = 663.82) | | (C53H37NS = 719.95) |
| 173 | m/z = 703.29 | 225 | m/z = 703.29 |
| | (C53H37NO = 703.88) | | (C53H37NO = 703.88) |
| 277 | m/z = 703.29 | 309 | m/z = 719.26 |
| | (C53H37NO = 703.88) | | (C53H37NS = 719.95) |
| 329 | m/z = 703.29 | 361 | m/z = 719.26 |
| | (C53H37NO = 703.88) | | (C53H37NS = 719.95) |
| 381 | m/z = 703.29 | 433 | m/z = 703.29 |
| | (C53H37NO = 703.88) | | (C53H37NO = 703.88) |
| 485 | m/z = 703.29 | 537 | m/z = 703.29 |
| | (C53H37NO = 703.88) | | (C53H37NO = 703.88) |
| 589 | m/z = 703.29 | 627 | m/z = 708.32 |
| | (C53H37NO = 703.88) | | (C53H32D5NO = 708.92) |
| 655 | m/z = 708.32 | | |
| | (C53H32D5NO = 708.92) | | |

### EXPERIMENTAL EXAMPLES

### Experimental Example 1

### 1) Manufacture of Organic Light-Emitting Device

The transparent electrode ITO thin film obtained from the glass for OLED (manufactured by Samsung-Corning) was subject to ultrasonic washing for each 5 minutes using trichloroethylene, acetone, ethanol, and distilled water sequentially, and then stored in isopropanol before use. Next, the ITO substrate was installed in the substrate folder of the vacuum deposition equipment, and the following 4,4′,4″-tris(N,N-(2-naphthyl)-phenylamino) triphenyl amine (2-TNATA) was placed in the cell in the vacuum deposition equipment:

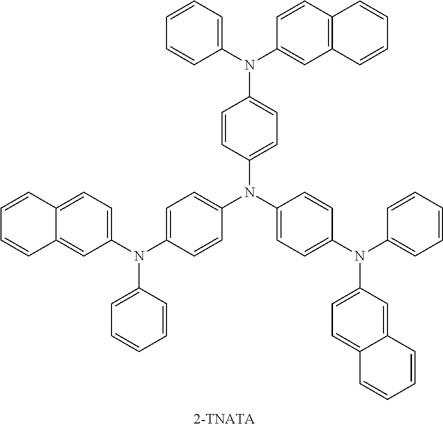 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Next, after evacuating the chamber until the vacuum degree reached 10⁻⁶ torr, an electric current was applied to the cell to evaporate 2-TNATA, thereby depositing a 600 Å-thick hole injection layer on the ITO substrate. The following N,N′-bis(α-naphthyl)-N,N′-diphenyl-4,4′-diamine (NPB) was placed in another cell in the vacuum deposition equipment and evaporated by applying an electric current to the cell, thereby depositing a 300 Å-thick hole transport layer on the hole injection layer:

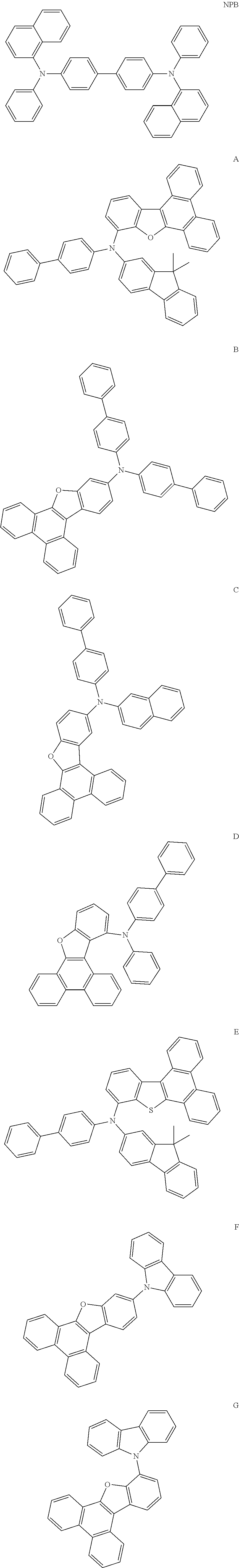

After the hole injection layer and the hole transport layer were formed in this way, a blue light-emitting material having the following structure was deposited thereon as a light-emitting layer. Specifically, the blue light-emitting host material H1 was vacuum-deposited to a thickness of 200 Å in one cell in the vacuum deposition equipment, and the blue light-emitting dopant material, D1 was vacuum-deposited at 5 wt % thereon relative to the host material.

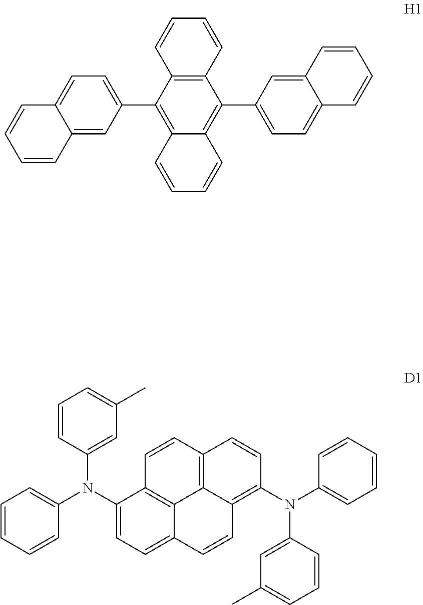

Next, an electron transport layer was deposited to a thickness of 300 Å with the compounds of following Structural Formula E1:

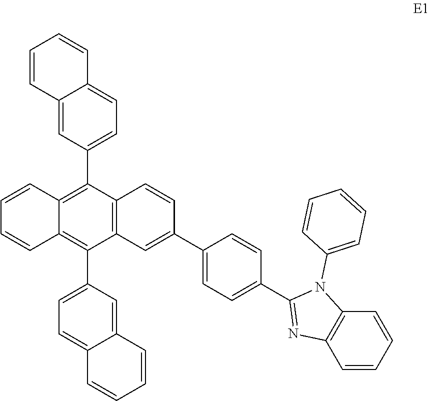 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An electron injection layer was deposited to a thickness of 10 Å with lithium fluoride (LiF) and an Al cathode was deposited to a thickness of 1,000 Å, thereby manufacturing an OLED device. On the other hand, all organic compounds required for manufacturing OLED devices were vacuum sublimated and purified under 10⁻⁶ to 10⁻⁸ torr for each material before use in OLED manufacturing.

Organic light-emitting devices of examples and comparative examples of the present invention were manufactured in the same manner as above, except that the compounds of the present invention shown in Table 4 below and Compounds A to G shown above were used instead of NPB used in forming the hole transport layer in the above. For the organic light-emitting device manufactured as described above, electroluminescence (EL) properties were measured with M7000 from McScience, and based on the measured results, T95 was measured when the reference luminance was 700 cd/m² through the lifetime measuring device (M6000) manufactured by McScience. The measured results of the driving voltage, luminous efficiency, color coordinates (CIE), and lifetime of the blue organic light-emitting device manufactured above are as shown in Table 4 below.

TABLE 4

| | | Driving | Luminous | | |
|---|---|---|---|---|---|
| | | Voltage | Efficiency | CIE | Lifetime |
| | Compound | (V) | (cd/A) | (x, y) | (T95) |
| Example 1 | 1 | 4.84 | 6.88 | (0.132, 0.103) | 70 |
| Example 2 | 17 | 4.60 | 6.90 | (0.133, 0.102) | 85 |
| Example 3 | 18 | 4.50 | 6.60 | (0.133, 0.100) | 90 |
| Example 4 | 21 | 4.55 | 6.70 | (0.133, 0.103) | 95 |
| Example 5 | 22 | 4.55 | 6.85 | (0.134, 0.101) | 90 |
| Example 6 | 23 | 4.88 | 6.55 | (0.131, 0.102) | 98 |
| Example 7 | 24 | 4.90 | 6.58 | (0.134, 0.101) | 65 |
| Example 8 | 25 | 4.70 | 6.68 | (0.132, 0.103) | 90 |
| Example 9 | 26 | 4.85 | 6.70 | (0.133, 0.103) | 62 |
| Example 10 | 29 | 4.60 | 6.76 | (0.133, 0.100) | 90 |
| Example 11 | 33 | 4.70 | 6.73 | (0.131, 0.103) | 85 |
| Example 12 | 34 | 4.84 | 6.75 | (0.133, 0.100) | 90 |
| Example 13 | 35 | 4.65 | 6.73 | (0.133, 0.102) | 85 |
| Example 14 | 36 | 4.72 | 6.76 | (0.132, 0.101) | 82 |
| Example 15 | 42 | 4.74 | 6.80 | (0.133, 0.100) | 83 |
| Example 16 | 45 | 4.78 | 6.85 | (0.133, 0.102) | 81 |
| Example 17 | 49 | 4.85 | 6.74 | (0.134, 0.103) | 74 |
| Example 18 | 69 | 4.95 | 6.72 | (0.135, 0.103) | 68 |
| Example 19 | 70 | 4.90 | 6.70 | (0.134, 0.102) | 70 |
| Example 20 | 121 | 4.75 | 6.88 | (0.134, 0.100) | 78 |
| Example 21 | 122 | 4.63 | 6.75 | (0.133, 0.102) | 81 |
| Example 22 | 153 | 4.71 | 6.60 | (0.132, 0.102) | 67 |
| Example 23 | 173 | 4.83 | 6.73 | (0.133, 0.101) | 69 |
| Example 24 | 225 | 4.80 | 6.70 | (0.131, 0.102) | 72 |
| Example 25 | 277 | 4.73 | 6.75 | (0.133, 0.101) | 82 |
| Example 26 | 309 | 4.80 | 6.63 | (0.133, 0.102) | 75 |
| Example 27 | 329 | 4.72 | 6.80 | (0.133, 0.100) | 82 |
| Example 28 | 361 | 4.83 | 6.65 | (0.133, 0.102) | 72 |
| Example 29 | 381 | 4.78 | 6.72 | (0.134, 0.103) | 75 |
| Example 30 | 433 | 4.80 | 6.74 | (0.133, 0.101) | 76 |
| Example 31 | 485 | 4.85 | 6.73 | (0.134, 0.104) | 78 |
| Example 32 | 537 | 4.79 | 6.70 | (0.133, 0.102) | 81 |
| Example 33 | 589 | 4.89 | 6.70 | (0.134, 0.104) | 74 |
| Example 34 | 627 | 4.62 | 6.80 | (0.131, 0.101) | 98 |
| Example 35 | 655 | 4.71 | 6.74 | (0.132, 0.101) | 95 |
| Comparative | NPB | 5.30 | 6.06 | (0.134, 0.101) | 51 |
| Example 1 | | | | | |
| Comparative | A | 5.14 | 6.23 | (0.133, 0.102) | 58 |
| Example 2 | | | | | |
| Comparative | B | 5.23 | 6.30 | (0.134, 0.101) | 59 |
| Example 3 | | | | | |
| Comparative | C | 5.30 | 6.18 | (0.131, 0.101) | 51 |
| Example 4 | | | | | |
| Comparative | D | 5.07 | 6.03 | (0.132, 0.102) | 48 |
| Example 5 | | | | | |
| Comparative | E | 5.15 | 6.15 | (0.133, 0.102) | 50 |
| Example 6 | | | | | |
| Comparative | F | 5.58 | 5.98 | (0.135, 0.103) | 45 |
| Example 7 | | | | | |
| Comparative | G | 5.50 | 6.13 | (0.134, 0.101) | 52 |
| Example 8 | | | | | |

From the results of Table 4, it can be confirmed that the blue organic light-emitting devices using the heterocyclic compound of the present invention as a material for a hole transport layer provide remarkably improved driving voltage, luminous efficiency, and lifetime properties, compared to the blue organic light-emitting devices of Comparative Examples 1 to 8 using NPB and Compounds A to G as a material for a hole transport layer.

The NPB used in the organic light-emitting device of Comparative Example 1 is similar to the heterocyclic compound of the present invention in that it has an arylamine group, but does not include a disubstituted dibenzofuran structure unlike the heterocyclic compound of the present invention. Therefore, due to such a structural difference, the organic light-emitting devices of the present invention exhibited remarkably superior effects in all aspects of driving voltage, luminous efficiency, and lifetime properties compared to the organic light-emitting device of Comparative Example 1.

Compounds A to G of Comparative Examples 2 to 8 have a structural difference from the heterocyclic compound of the present invention including a disubstituted dibenzofuran structure in that they include a monosubstituted dibenzofuran structure having one substituent. In the case of monosubstituted dibenzofuran, pi-pi stacking of the aromatic ring occurs, which increases the driving voltage, thereby being able to degrade device properties. On the other hand, in the case of disubstituted dibenzofuran, pi-pi stacking of the aromatic ring is suppressed, thereby exhibiting an effect of suppressing deterioration of device properties due to an increase in driving voltage of an organic light-emitting device. Thus, the heterocyclic compound of the present invention including such a disubstituted dibenzofuran structure provides significantly improved hole transport properties or stability compared to the compounds of Comparative Examples 2 to 8 including a monosubstituted dibenzofuran structure. In addition, due to these effects, the organic light-emitting device of the present invention including a hole transport layer formed using the heterocyclic compound of the present invention provides very excellent driving voltage, luminous efficiency, and lifetime properties, compared to the organic light-emitting devices of Comparative Examples 2 to 8.

### Experimental Example 2

### 1) Manufacture of Organic Light-Emitting Devices

The transparent electrode ITO thin film obtained from the glass for OLED (manufactured by Samsung-Corning) was subject to ultrasonic washing for each 5 minutes using trichloroethylene, acetone, ethanol, and distilled water sequentially, and then stored in isopropanol before use. Next, the ITO substrate was installed in the substrate folder of the vacuum deposition equipment, and the following 4,4′,4″-tris(N,N-(2-naphthyl)-phenylamino) triphenylamine (2-TNATA) was placed in the cell in the vacuum deposition equipment:

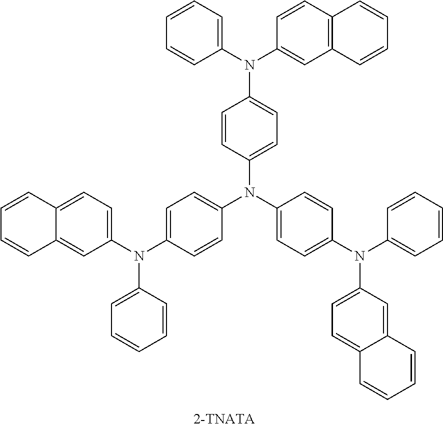 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Next, after evacuating the chamber until the vacuum degree reached 10⁻⁶ torr, an electric current was applied to the cell to evaporate 2-TNATA, thereby depositing a 600 Å-thick hole injection layer on the ITO substrate. The following N,N′-bis(α-naphthyl)-N,N′-diphenyl-4,4′-diamine (NPB) was placed in another cell in the vacuum deposition equipment and evaporated by applying an electric current to the cell, thereby depositing a 300 Å-thick hole transport layer on the hole injection layer:

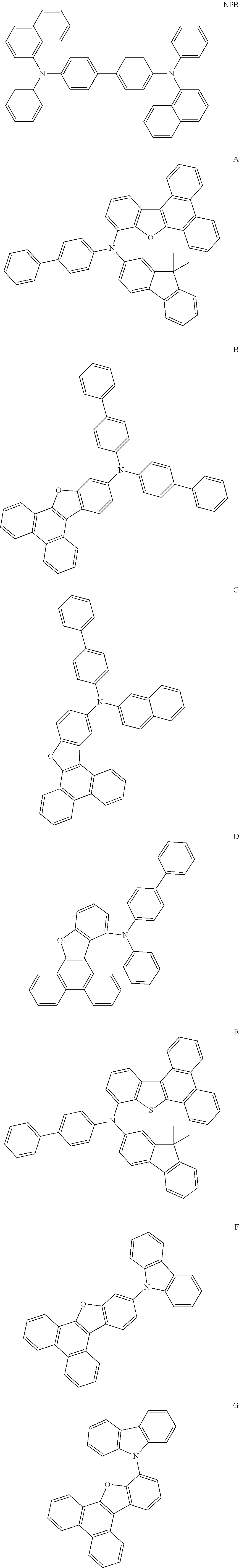

After the hole injection layer and the hole transport layer were formed in this way, a blue light-emitting material having the following structure was deposited thereon as a light-emitting layer. Specifically, the blue light-emitting host material, H1 was vacuum-deposited to a thickness of 200 Å in one cell in the vacuum deposition equipment, and the blue light-emitting dopant material, D1 was vacuum-deposited at 5 wt % thereon relative to the host material.

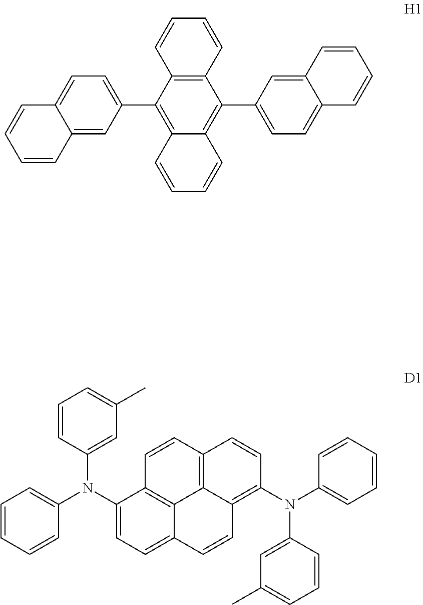

Next, an electron transport layer was deposited to a thickness of 300 Å with the compounds of following Structural Formula E1:

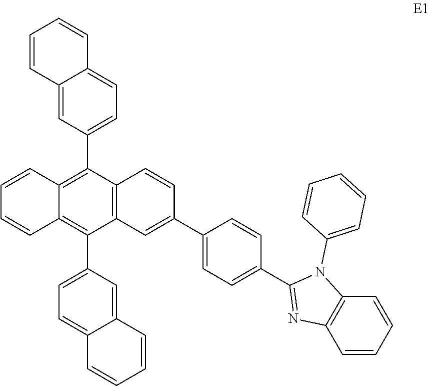 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An electron injection layer was deposited to a thickness of 10 Å with lithium fluoride (LiF) and an Al cathode was deposited to a thickness of 1,000 Å, thereby manufacturing an OLED device. On the other hand, all organic compounds required for manufacturing OLED devices were vacuum sublimated and purified under 10⁻⁶ to 10⁻⁸ torr for each material before use in OLED manufacturing.

Organic light-emitting devices of examples and comparative examples of the present invention were manufactured in the same manner as above, except that the hole transport layer NPB was formed to a thickness of 250 Å, and then an electron-blocking layer was formed by depositing the heterocyclic compound of the present invention shown in Table 5 below and Compounds A to G shown above to a thickness of 50 Å on the hole transport layer. The measured results of the driving voltage, luminous efficiency, color coordinates (CIE), and lifetime of the blue organic light-emitting device manufactured above are as shown in Table 5 below.

TABLE 5

| | | Driving | Luminous | | |
|---|---|---|---|---|---|
| | | Voltage | Efficiency | CIE | Lifetime |
| | Compound | (V) | (cd/A) | (x, y) | (T95) |
| Example 36 | 1 | 5.14 | 5.95 | (0.134, 0.101) | 73 |
| Example 37 | 17 | 5.20 | 6.15 | (0.133, 0.101) | 80 |
| Example 38 | 18 | 5.24 | 6.18 | (0.134, 0.102) | 83 |
| Example 39 | 21 | 5.26 | 6.24 | (0.134, 0.101) | 85 |
| Example 40 | 22 | 4.99 | 6.25 | (0.133, 0.101) | 80 |
| Example 41 | 23 | 5.30 | 6.15 | (0.132, 0.102) | 88 |
| Example 42 | 24 | 5.34 | 6.10 | (0.134, 0.103) | 75 |
| Example 43 | 25 | 5.28 | 6.20 | (0.133, 0.101) | 85 |
| Example 44 | 26 | 5.32 | 6.15 | (0.134, 0.102) | 74 |
| Example 45 | 29 | 4.85 | 6.29 | (0.132, 0.101) | 83 |
| Example 46 | 33 | 4.93 | 6.30 | (0.133, 0.100) | 78 |
| Example 47 | 34 | 5.13 | 6.23 | (0.134, 0.102) | 80 |
| Example 48 | 35 | 5.14 | 6.28 | (0.134, 0.101) | 82 |
| Example 49 | 36 | 4.94 | 6.28 | (0.133, 0.100) | 75 |
| Example 50 | 42 | 4.95 | 6.30 | (0.134, 0.100) | 77 |
| Example 51 | 45 | 5.02 | 6.36 | (0.132, 0.101) | 73 |
| Example 52 | 49 | 5.23 | 6.16 | (0.134, 0.103) | 70 |
| Example 53 | 69 | 5.28 | 6.20 | (0.133, 0.101) | 68 |
| Example 54 | 70 | 5.30 | 6.30 | (0.134, 0.100) | 68 |
| Example 55 | 121 | 4.98 | 6.31 | (0.134, 0.101) | 75 |
| Example 56 | 122 | 5.00 | 6.23 | (0.132, 0.101) | 79 |
| Example 57 | 153 | 5.17 | 6.20 | (0.134, 0.102) | 66 |
| Example 58 | 173 | 5.13 | 6.30 | (0.133, 0.101) | 68 |
| Example 59 | 225 | 5.11 | 6.23 | (0.132, 0.101) | 66 |
| Example 60 | 277 | 4.98 | 6.28 | (0.133, 0.100) | 73 |
| Example 61 | 309 | 5.20 | 6.18 | (0.133, 0.102) | 70 |
| Example 62 | 329 | 5.10 | 6.25 | (0.133, 0.100) | 78 |
| Example 63 | 361 | 5.24 | 6.15 | (0.134, 0.102) | 70 |
| Example 64 | 381 | 5.13 | 6.28 | (0.134, 0.100) | 73 |
| Example 65 | 433 | 5.25 | 6.18 | (0.133, 0.101) | 70 |
| Example 66 | 485 | 5.15 | 6.20 | (0.132, 0.101) | 71 |
| Example 67 | 537 | 5.14 | 6.18 | (0.133, 0.100) | 70 |
| Example 68 | 589 | 5.18 | 6.22 | (0.132, 0.102) | 71 |
| Example 69 | 627 | 4.93 | 6.35 | (0.131, 0.100) | 93 |
| Example 70 | 655 | 5.00 | 6.25 | (0.132, 0.100) | 89 |
| Comparative | NPB | 5.83 | 5.57 | (0.133, 0.101) | 49 |
| Example 9 | | | | | |
| Comparative | A | 5.70 | 5.73 | (0.131, 0.101) | 51 |
| Example 10 | | | | | |
| Comparative | B | 5.78 | 5.85 | (0.132, 0.100) | 54 |
| Example 11 | | | | | |
| Comparative | C | 5.85 | 5.71 | (0.131, 0.101) | 45 |
| Example 12 | | | | | |
| Comparative | D | 5.71 | 5.67 | (0.133, 0.102) | 49 |
| Example 13 | | | | | |
| Comparative | E | 5.85 | 5.65 | (0.134, 0.101) | 52 |
| Example 14 | | | | | |
| Comparative | F | 5.73 | 5.63 | (0.132, 0.100) | 43 |
| Example 15 | | | | | |
| Comparative | G | 5.75 | 5.65 | (0.133, 0.100) | 45 |
| Example 16 | | | | | |

From the results of Table 5, it can be confirmed that the blue organic light-emitting devices using the heterocyclic compound of the present invention as a material for an electron-blocking layer are remarkably improved in all aspects of driving voltage, luminous efficiency, and lifetime properties, compared to the blue organic light-emitting devices of Comparative Examples 9 and 10 to 16 using NPB and Compounds A to G as a material for an electron-blocking layer.

In the organic light-emitting device, when electrons pass through the hole transport layer to the anode without being combined in the light-emitting layer, the efficiency and lifetime of the OLED device are reduced. In order to prevent this phenomenon, a compound having a high LUMO level is used as the electron-blocking layer, and in this case, electrons passing through the light-emitting layer to the anode are blocked by the energy barrier of the electron-blocking layer. Therefore, the probability that holes and electrons form excitons increases, and the possibility that they are emitted as light in the light-emitting layer increases.

As confirmed in Experimental Example 2 above, the heterocyclic compound of the present invention exhibits excellent electron-blocking performance compared to NPB and Compounds A to G when used as a material for an electron-blocking layer. In addition, the organic light-emitting device of the present invention including an electron-blocking layer formed by such a heterocyclic compound of the present invention provides remarkably excellent driving voltage, luminous efficiency, and lifetime properties, compared to the organic light-emitting devices of Comparative Examples 9 to 16.

### DESCRIPTION OF SYMBOLS

| | |
|---|---|
| 100: Substrate | 200: Anode |
| 300: Organic layer | 301: Hole injection layer |
| 302: Hole transport layer | 303: Light-emitting layer |
| 304: Hole-blocking layer | 305: Electron transport layer |
| 306: Electron injection layer | 400: Cathode |

1. A heterocyclic compound represented by following Formula 1: 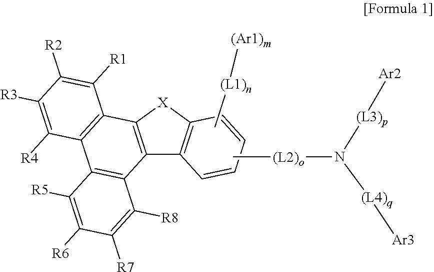
wherein,
X is O or S,
Ar1, Ar2, and Ar3 are the same as or different from each other and are each independently a substituted or unsubstituted C6 to C60 aryl group or a substituted or unsubstituted C2 to C60 heteroaryl group,
R1 to R8 are the same as or different from each other and are each independently hydrogen: deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group: or —NR21R22, wherein R21 and R22 are the same as or different from each other and are each independently a substituted or unsubstituted C1 to C10 alkyl group, a substituted or unsubstituted C6 to C60 aryl group, or a substituted or unsubstituted C2 to C60 heteroaryl group; and R21 and R22 above may be combined with each other to form a substituted or unsubstituted C6 to C60 aromatic hydrocarbon ring or a substituted or unsubstituted C2 to C60 heterocycle,
L1 to L4 are the same as or different from each other and are each independently a direct bond, a substituted or unsubstituted C6 to C60 arylene group, or a substituted or unsubstituted C2 to C60 heteroarylene group,
m is an integer from 1 to 3, with the proviso that when m is 2 or more, each Ar1 is the same as or different from each other,
n, o, p, and q are the same as or different from each other and are each independently an integer from 0 to 3, with the proviso that when each of n, o, p, and q is 2 or more, each of L1, L2, L3, and L4 is the same as or different from each other.

2. The heterocyclic compound according to claim 1, characterized in that Ar1, Ar2, and Ar3 are the same as or different from each other and are each independently a substituted or unsubstituted C6 to C30 aryl group or a substituted or unsubstituted C2 to C30 heteroaryl group.

3. The heterocyclic compound according to claim 1, characterized in that R1 to R8 are the same as or different from each other and are each independently hydrogen; deuterium; a substituted or unsubstituted C1 to C20 alkyl group; a substituted or unsubstituted C1 to C20 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; or —NR21R22, wherein R21 and R22 are the same as or different from each other and are each independently a substituted or unsubstituted C1 to C10 alkyl group, a substituted or unsubstituted C6 to C30 aryl group, or a substituted or unsubstituted C2 to C30 heteroaryl group; and R21 and R22 above may be combined with each other to form a substituted or unsubstituted C6 to C30 aromatic hydrocarbon ring or a substituted or unsubstituted C2 to C30 heterocycle.

4. The heterocyclic compound according to claim 1, characterized in that
X is O or S,
Ar1, Ar2, and Ar3 are the same as or different from each other and are each independently a substituted or unsubstituted C6 to C30 aryl group or a substituted or unsubstituted C2 to C30 heteroaryl group,
R1 to R8 are the same as or different from each other and are each independently hydrogen; deuterium; a substituted or unsubstituted C6 to C30 aryl group; a substituted or unsubstituted C2 to C30 heteroaryl group; or —NR21R22, wherein R21 and R22 are the same as or different from each other and are each independently a substituted or unsubstituted C1 to C10 alkyl group, a substituted or unsubstituted C6 to C30 aryl group, or a substituted or unsubstituted C2 to C30 heteroaryl group, and
L1 to L4 are the same as or different from each other and are each independently a direct bond, a substituted or unsubstituted C6 to C30 arylene group, or a substituted or unsubstituted C2 to C30 heteroarylene group.

5. The heterocyclic compound according to claim 1, characterized in that the heterocyclic compound represented by Formula 1 above is a compound represented by any one of the following Formulas 2 to 5: 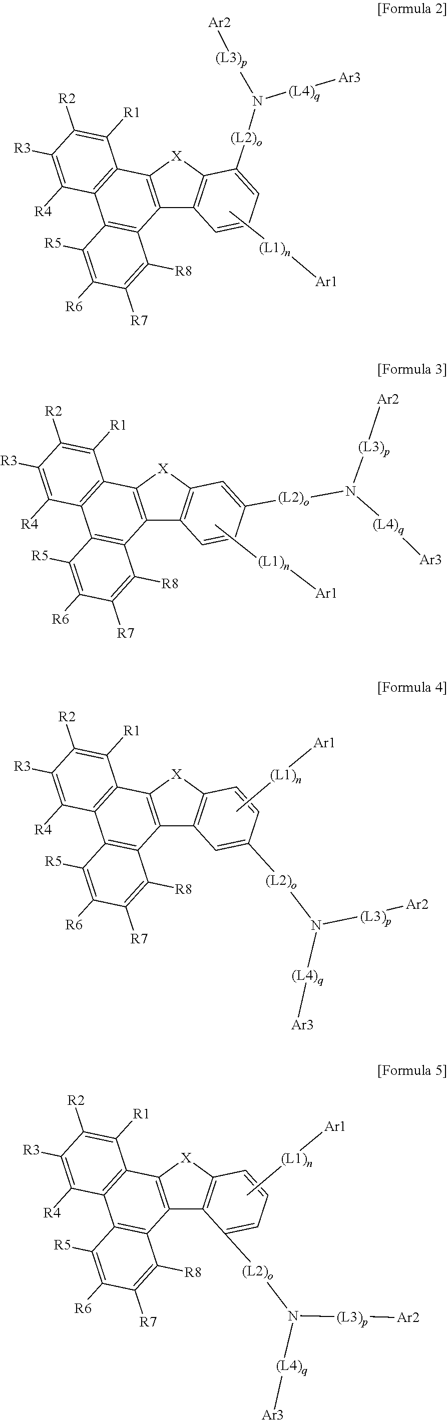
wherein,
X, Ar1, Ar2, Ar3, R1 to R8, L1 to L4, n, o, p, and q are as defined in claim 1.

6. The heterocyclic compound according to claim 1, characterized in that the heterocyclic compound represented by Formula 1 above is a compound represented by any one of the following compounds:

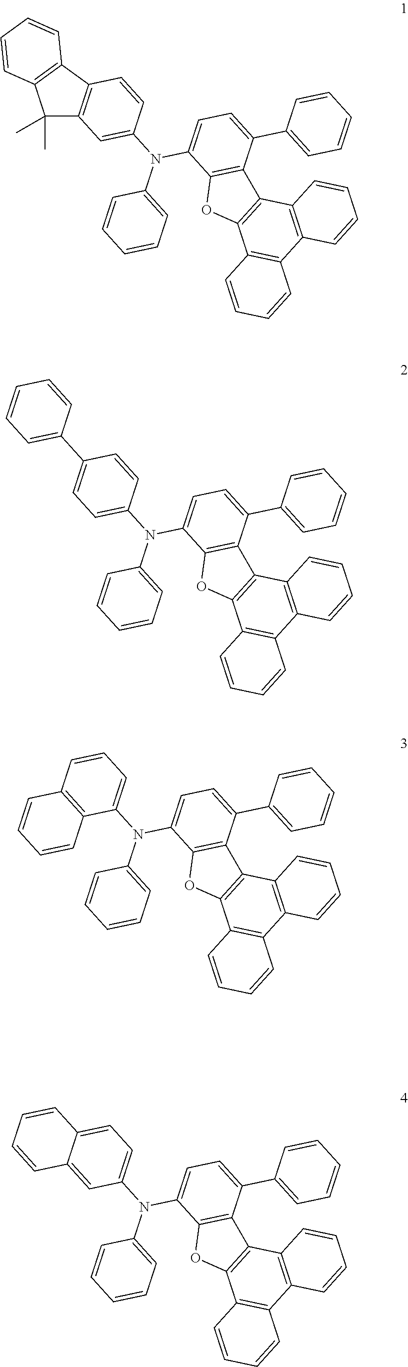 [SMILES: CC1(C)c2ccccc2-c2ccc(N(c3ccccc3)c3ccc(-c4ccccc4)c4c3oc3c5ccccc5c5ccccc5c34)cc21 ; c1ccc(-c2ccc(N(c3ccccc3)c3ccc(-c4ccccc4)c4c3oc3c5ccccc5c5ccccc5c34)cc2)cc1 ; c1ccc(-c2ccc(N(c3ccccc3)c3cccc4ccccc34)c3oc4c5ccccc5c5ccccc5c4c23)cc1 ; c1ccc(-c2ccc(N(c3ccccc3)c3ccc4ccccc4c3)c3oc4c5ccccc5c5ccccc5c4c23)cc1]

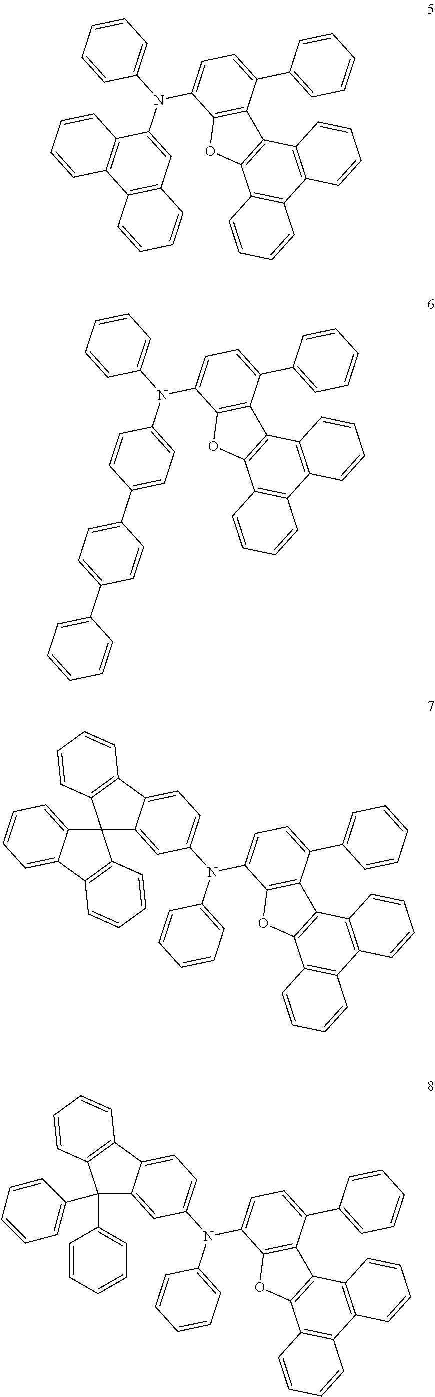 [SMILES: c1ccc(-c2ccc(N(c3ccccc3)c3cc4ccccc4c4ccccc34)c3oc4c5ccccc5c5ccccc5c4c23)cc1 ; c1ccc(-c2ccc(-c3ccc(N(c4ccccc4)c4ccc(-c5ccccc5)c5c4oc4c6ccccc6c6ccccc6c45)cc3)cc2)cc1 ; c1ccc(-c2ccc(N(c3ccccc3)c3ccc4c(c3)C3(c5ccccc5-c5ccccc53)c3ccccc3-4)c3oc4c5ccccc5c5ccccc5c4c23)cc1 ; c1ccc(-c2ccc(N(c3ccccc3)c3ccc4c(c3)C(c3ccccc3)(c3ccccc3)c3ccccc3-4)c3oc4c5ccccc5c5ccccc5c4c23)cc1]

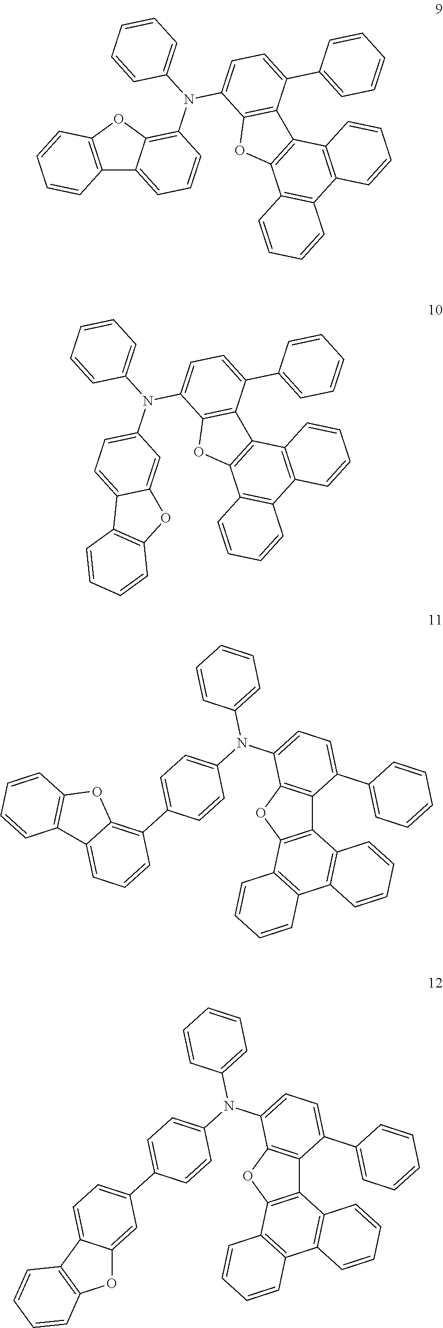 [SMILES: c1ccc(-c2ccc(N(c3ccccc3)c3cccc4c3oc3ccccc34)c3oc4c5ccccc5c5ccccc5c4c23)cc1 ; c1ccc(-c2ccc(N(c3ccccc3)c3ccc4c(c3)oc3ccccc34)c3oc4c5ccccc5c5ccccc5c4c23)cc1 ; c1ccc(-c2ccc(N(c3ccccc3)c3ccc(-c4cccc5c4oc4ccccc45)cc3)c3oc4c5ccccc5c5ccccc5c4c23)cc1 ; c1ccc(-c2ccc(N(c3ccccc3)c3ccc(-c4ccc5c(c4)oc4ccccc45)cc3)c3oc4c5ccccc5c5ccccc5c4c23)cc1]

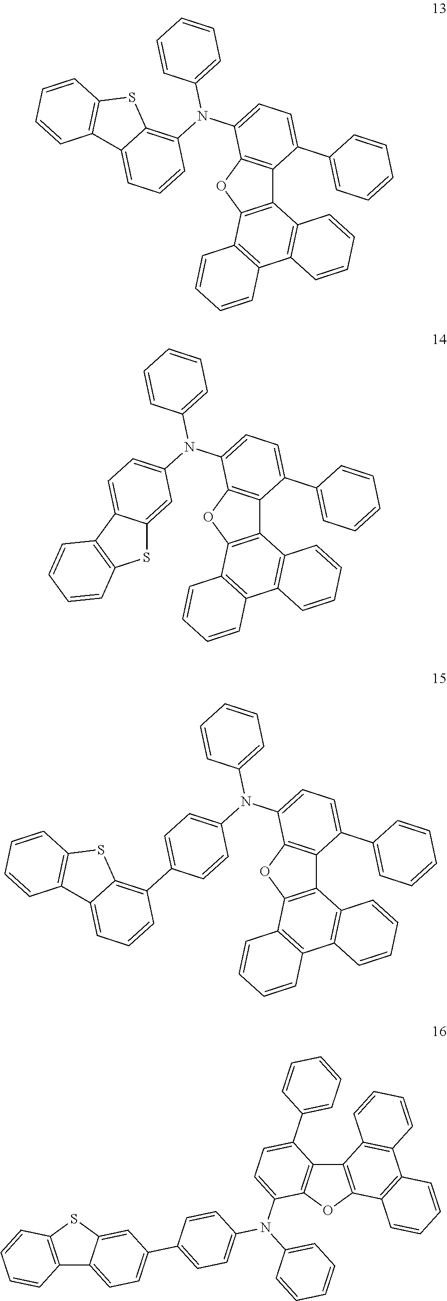 [SMILES: c1ccc(-c2ccc(N(c3ccccc3)c3cccc4c3sc3ccccc34)c3oc4c5ccccc5c5ccccc5c4c23)cc1 ; c1ccc(-c2ccc(N(c3ccccc3)c3ccc4c(c3)sc3ccccc34)c3oc4c5ccccc5c5ccccc5c4c23)cc1 ; c1ccc(-c2ccc(N(c3ccccc3)c3ccc(-c4cccc5c4sc4ccccc45)cc3)c3oc4c5ccccc5c5ccccc5c4c23)cc1 ; c1ccc(-c2ccc(N(c3ccccc3)c3ccc(-c4ccc5c(c4)sc4ccccc45)cc3)c3oc4c5ccccc5c5ccccc5c4c23)cc1]

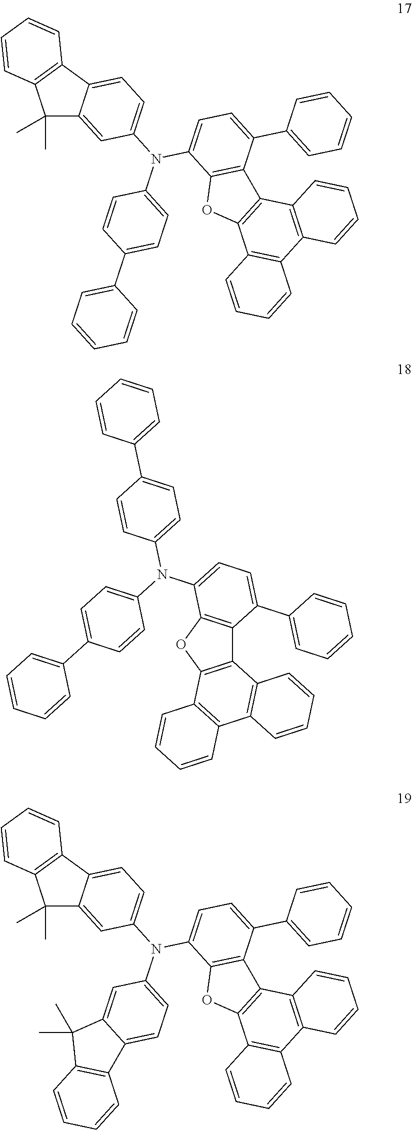

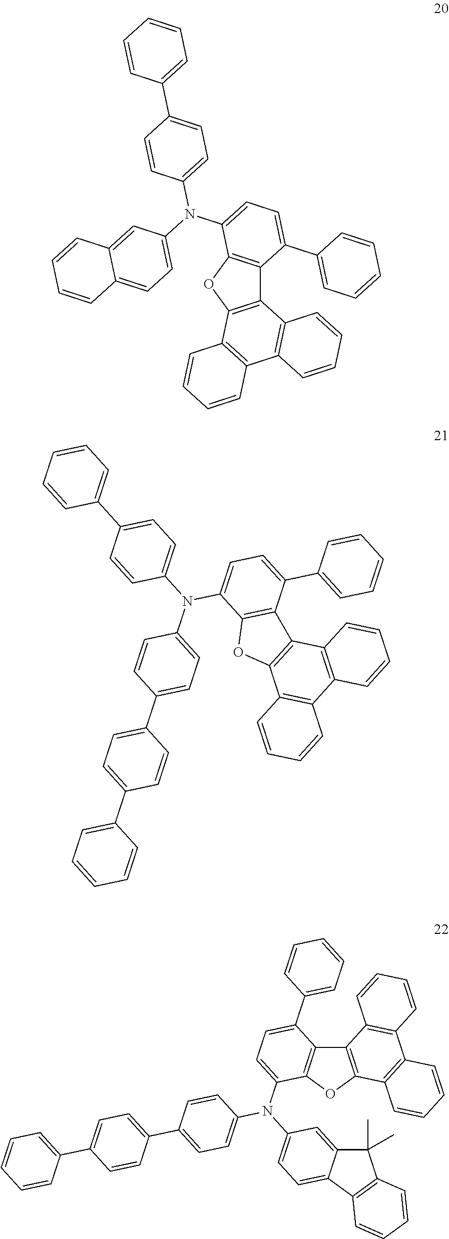 [SMILES: c1ccc(-c2ccc(N(c3ccc4ccccc4c3)c3ccc(-c4ccccc4)c4c3oc3c5ccccc5c5ccccc5c34)cc2)cc1 ; c1ccc(-c2ccc(-c3ccc(N(c4ccc(-c5ccccc5)cc4)c4ccc(-c5ccccc5)c5c4oc4c6ccccc6c6ccccc6c45)cc3)cc2)cc1 ; CC1(C)c2ccccc2-c2ccc(N(c3ccc(-c4ccc(-c5ccccc5)cc4)cc3)c3ccc(-c4ccccc4)c4c3oc3c5ccccc5c5ccccc5c34)cc21]

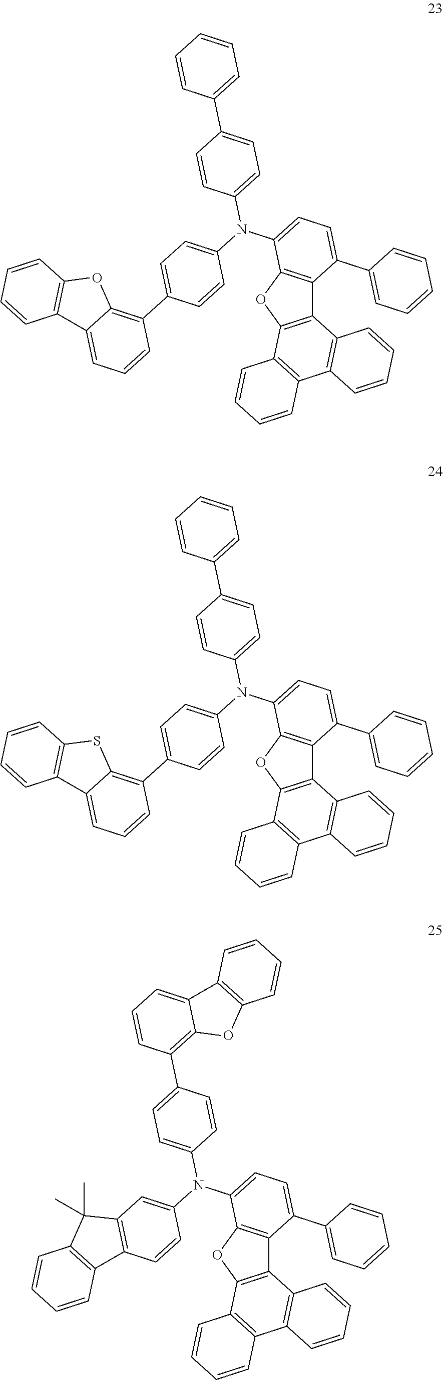 [SMILES: c1ccc(-c2ccc(N(c3ccc(-c4cccc5c4oc4ccccc45)cc3)c3ccc(-c4ccccc4)c4c3oc3c5ccccc5c5ccccc5c34)cc2)cc1 ; c1ccc(-c2ccc(N(c3ccc(-c4cccc5c4sc4ccccc45)cc3)c3ccc(-c4ccccc4)c4c3oc3c5ccccc5c5ccccc5c34)cc2)cc1 ; CC1(C)c2ccccc2-c2ccc(N(c3ccc(-c4cccc5c4oc4ccccc45)cc3)c3ccc(-c4ccccc4)c4c3oc3c5ccccc5c5ccccc5c34)cc21]

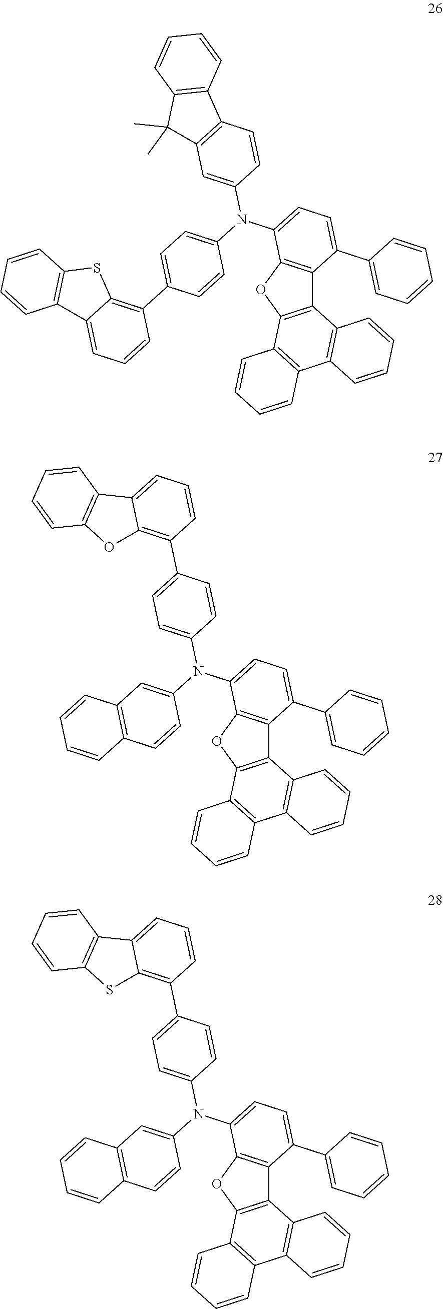 [SMILES: CC1(C)c2ccccc2-c2ccc(N(c3ccc(-c4cccc5c4sc4ccccc45)cc3)c3ccc(-c4ccccc4)c4c3oc3c5ccccc5c5ccccc5c34)cc21 ; c1ccc(-c2ccc(N(c3ccc(-c4cccc5c4oc4ccccc45)cc3)c3ccc4ccccc4c3)c3oc4c5ccccc5c5ccccc5c4c23)cc1 ; c1ccc(-c2ccc(N(c3ccc(-c4cccc5c4sc4ccccc45)cc3)c3ccc4ccccc4c3)c3oc4c5ccccc5c5ccccc5c4c23)cc1]

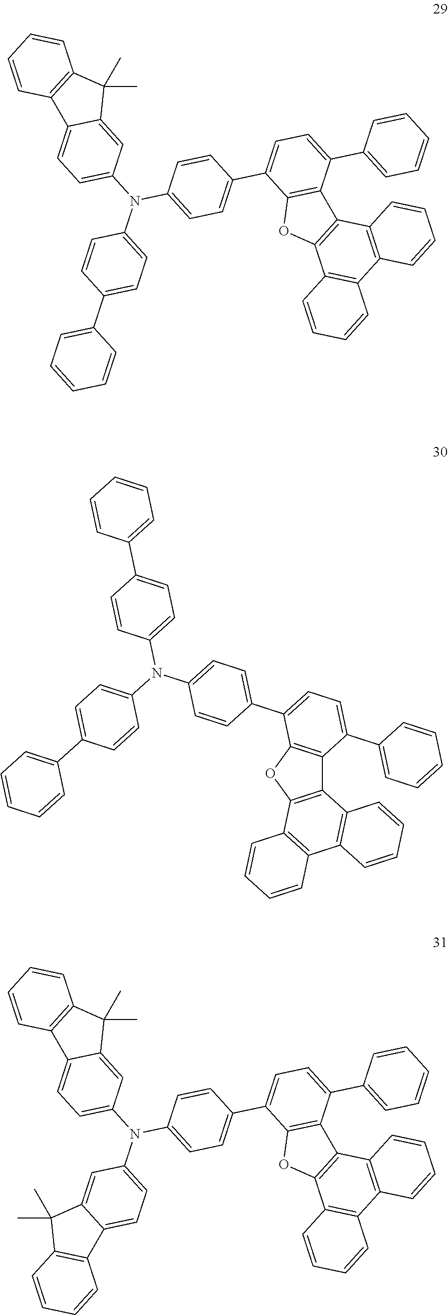 [SMILES: CC1(C)c2ccccc2-c2ccc(N(c3ccc(-c4ccccc4)cc3)c3ccc(-c4ccc(-c5ccccc5)c5c4oc4c6ccccc6c6ccccc6c45)cc3)cc21 ; c1ccc(-c2ccc(N(c3ccc(-c4ccccc4)cc3)c3ccc(-c4ccc(-c5ccccc5)c5c4oc4c6ccccc6c6ccccc6c45)cc3)cc2)cc1 ; CC1(C)c2ccccc2-c2ccc(N(c3ccc(-c4ccc(-c5ccccc5)c5c4oc4c6ccccc6c6ccccc6c45)cc3)c3ccc4c(c3)C(C)(C)c3ccccc3-4)cc21]

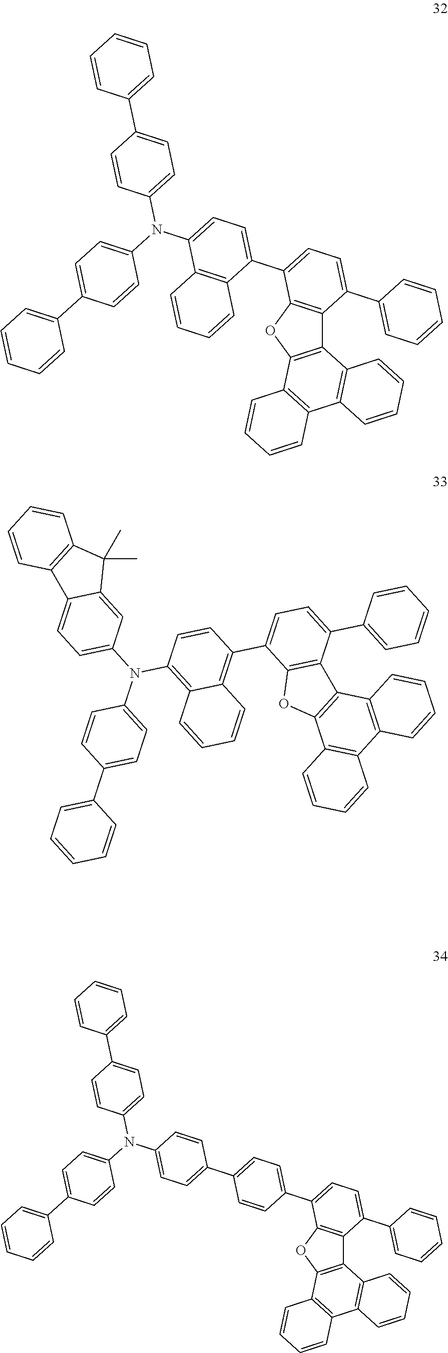 [SMILES: c1ccc(-c2ccc(N(c3ccc(-c4ccccc4)cc3)c3ccc(-c4ccc(-c5ccccc5)c5c4oc4c6ccccc6c6ccccc6c45)c4ccccc34)cc2)cc1 ; CC1(C)c2ccccc2-c2ccc(N(c3ccc(-c4ccccc4)cc3)c3ccc(-c4ccc(-c5ccccc5)c5c4oc4c6ccccc6c6ccccc6c45)c4ccccc34)cc21 ; c1ccc(-c2ccc(N(c3ccc(-c4ccccc4)cc3)c3ccc(-c4ccc(-c5ccc(-c6ccccc6)c6c5oc5c7ccccc7c7ccccc7c56)cc4)cc3)cc2)cc1]

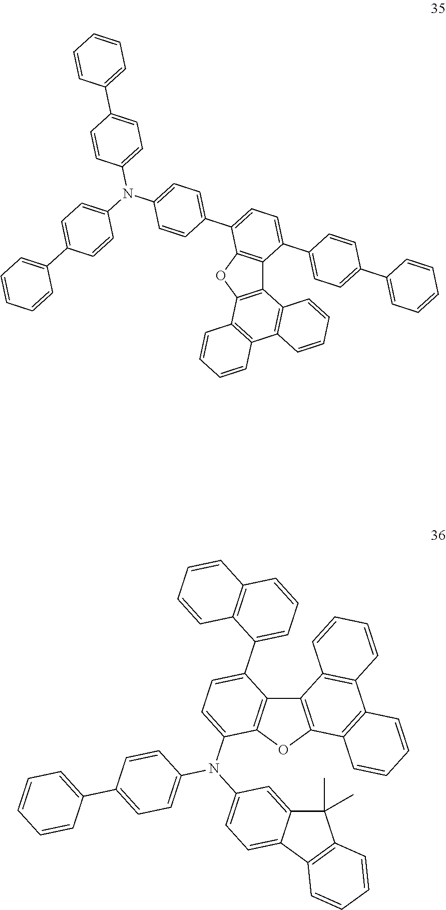 [SMILES: c1ccc(-c2ccc(-c3ccc(-c4ccc(N(c5ccc(-c6ccccc6)cc5)c5ccc(-c6ccccc6)cc5)cc4)c4oc5c6ccccc6c6ccccc6c5c34)cc2)cc1 ; CC1(C)c2ccccc2-c2ccc(N(c3ccc(-c4ccccc4)cc3)c3ccc(-c4cccc5ccccc45)c4c3oc3c5ccccc5c5ccccc5c34)cc21]

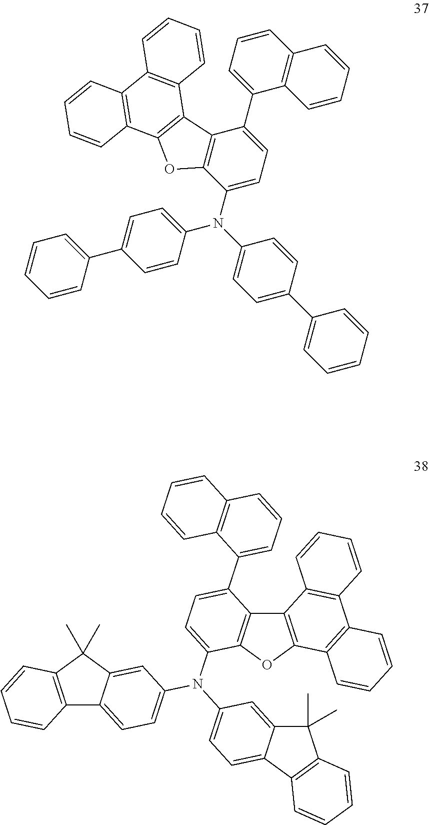 [SMILES: c1ccc(-c2ccc(N(c3ccc(-c4ccccc4)cc3)c3ccc(-c4cccc5ccccc45)c4c3oc3c5ccccc5c5ccccc5c34)cc2)cc1 ; CC1(C)c2ccccc2-c2ccc(N(c3ccc4c(c3)C(C)(C)c3ccccc3-4)c3ccc(-c4cccc5ccccc45)c4c3oc3c5ccccc5c5ccccc5c34)cc21]

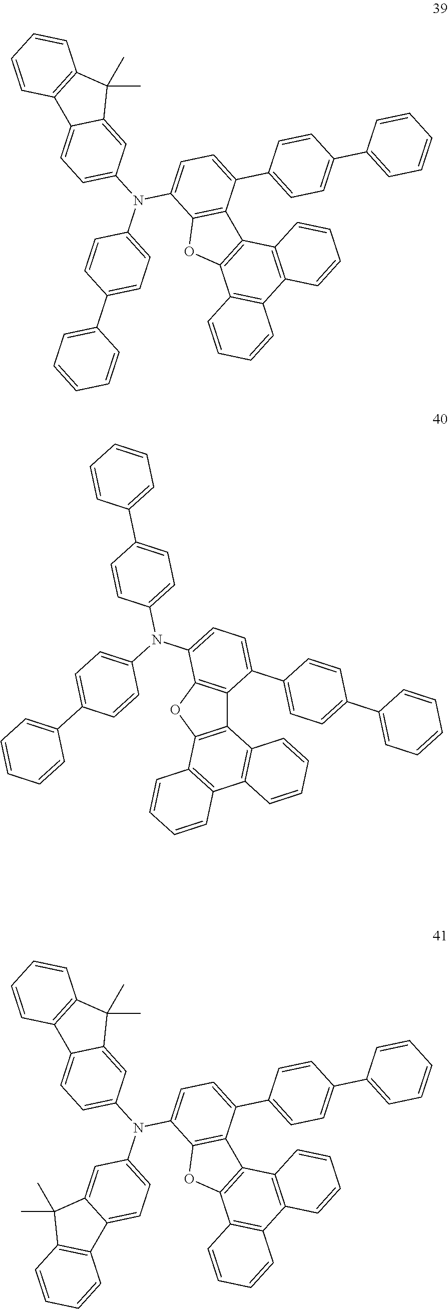 [SMILES: CC1(C)c2ccccc2-c2ccc(N(c3ccc(-c4ccccc4)cc3)c3ccc(-c4ccc(-c5ccccc5)cc4)c4c3oc3c5ccccc5c5ccccc5c34)cc21 ; c1ccc(-c2ccc(-c3ccc(N(c4ccc(-c5ccccc5)cc4)c4ccc(-c5ccccc5)cc4)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccc(-c5ccccc5)cc4)c4c3oc3c5ccccc5c5ccccc5c34)cc21]

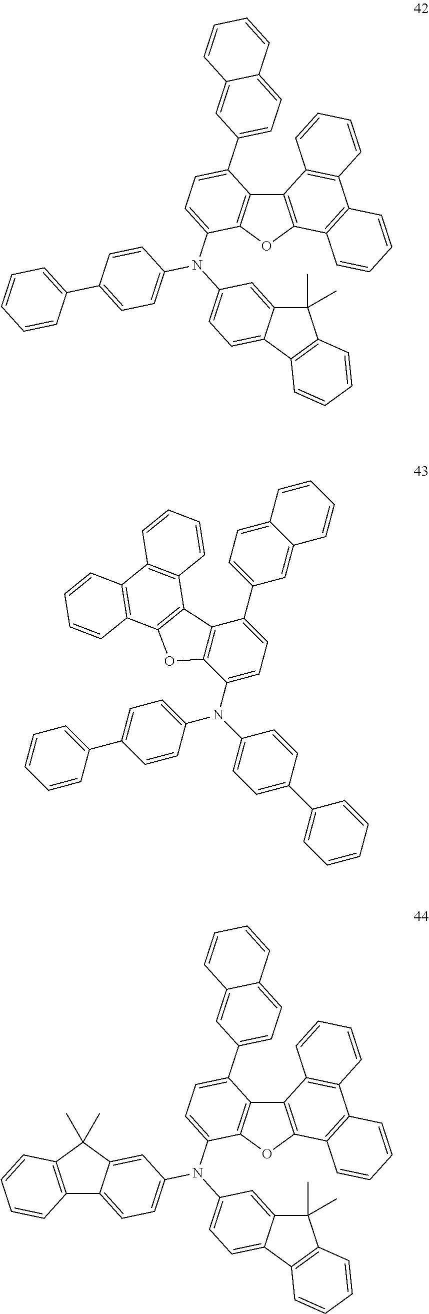 [SMILES: CC1(C)c2ccccc2-c2ccc(N(c3ccc(-c4ccccc4)cc3)c3ccc(-c4ccc5ccccc5c4)c4c3oc3c5ccccc5c5ccccc5c34)cc21 ; c1ccc(-c2ccc(N(c3ccc(-c4ccccc4)cc3)c3ccc(-c4ccc5ccccc5c4)c4c3oc3c5ccccc5c5ccccc5c34)cc2)cc1 ; CC1(C)c2ccccc2-c2ccc(N(c3ccc4c(c3)C(C)(C)c3ccccc3-4)c3ccc(-c4ccc5ccccc5c4)c4c3oc3c5ccccc5c5ccccc5c34)cc21]

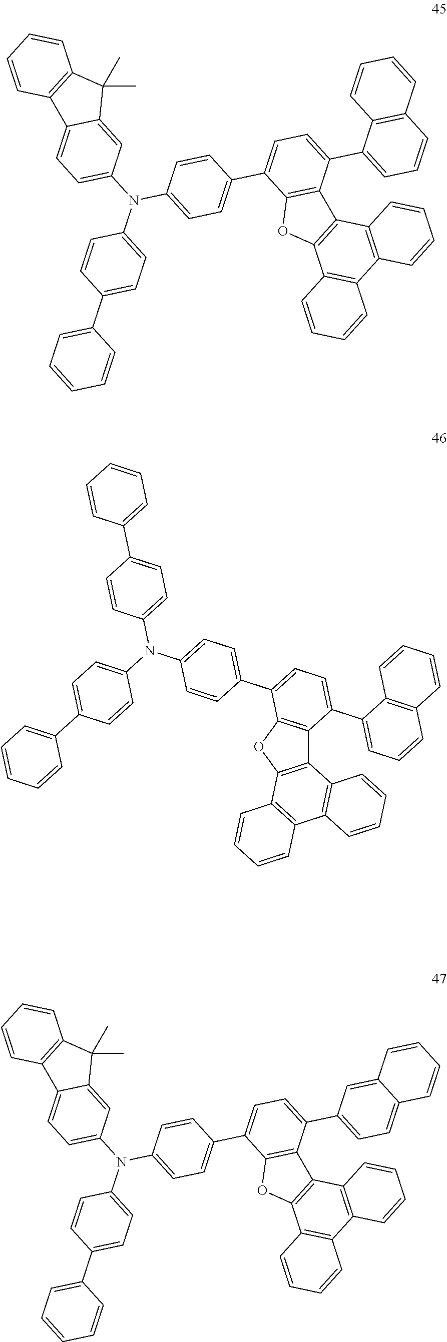 [SMILES: CC1(C)c2ccccc2-c2ccc(N(c3ccc(-c4ccccc4)cc3)c3ccc(-c4ccc(-c5cccc6ccccc56)c5c4oc4c6ccccc6c6ccccc6c45)cc3)cc21 ; c1ccc(-c2ccc(N(c3ccc(-c4ccccc4)cc3)c3ccc(-c4ccc(-c5cccc6ccccc56)c5c4oc4c6ccccc6c6ccccc6c45)cc3)cc2)cc1 ; CC1(C)c2ccccc2-c2ccc(N(c3ccc(-c4ccccc4)cc3)c3ccc(-c4ccc(-c5ccc6ccccc6c5)c5c4oc4c6ccccc6c6ccccc6c45)cc3)cc21]

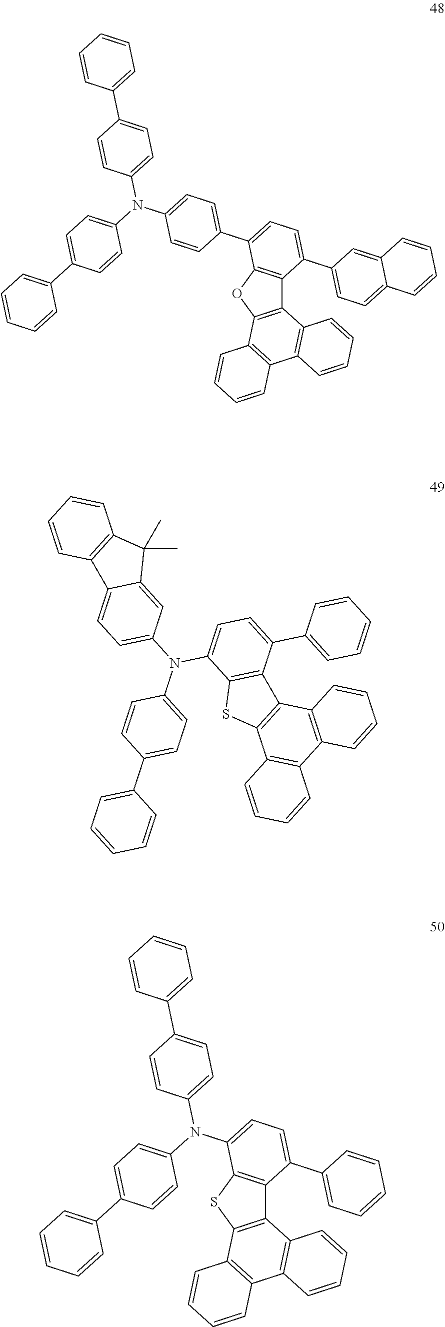 [SMILES: c1ccc(-c2ccc(N(c3ccc(-c4ccccc4)cc3)c3ccc(-c4ccc(-c5ccc6ccccc6c5)c5c4oc4c6ccccc6c6ccccc6c45)cc3)cc2)cc1 ; CC1(C)c2ccccc2-c2ccc(N(c3ccc(-c4ccccc4)cc3)c3ccc(-c4ccccc4)c4c3sc3c5ccccc5c5ccccc5c34)cc21 ; c1ccc(-c2ccc(N(c3ccc(-c4ccccc4)cc3)c3ccc(-c4ccccc4)c4c3sc3c5ccccc5c5ccccc5c34)cc2)cc1]

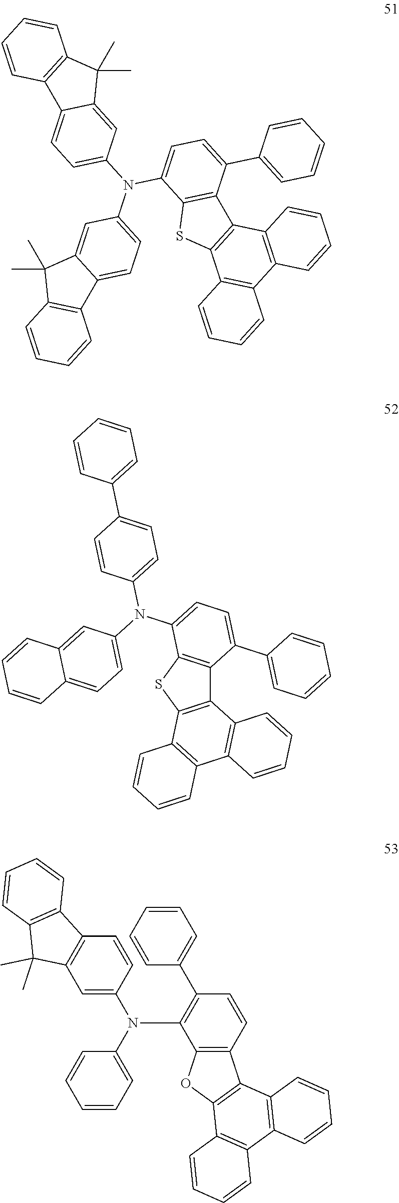 [SMILES: CC1(C)c2ccccc2-c2ccc(N(c3ccc4c(c3)C(C)(C)c3ccccc3-4)c3ccc(-c4ccccc4)c4c3sc3c5ccccc5c5ccccc5c34)cc21 ; c1ccc(-c2ccc(N(c3ccc4ccccc4c3)c3ccc(-c4ccccc4)c4c3sc3c5ccccc5c5ccccc5c34)cc2)cc1 ; CC1(C)c2ccccc2-c2ccc(N(c3ccccc3)c3c(-c4ccccc4)ccc4c3oc3c5ccccc5c5ccccc5c43)cc21]

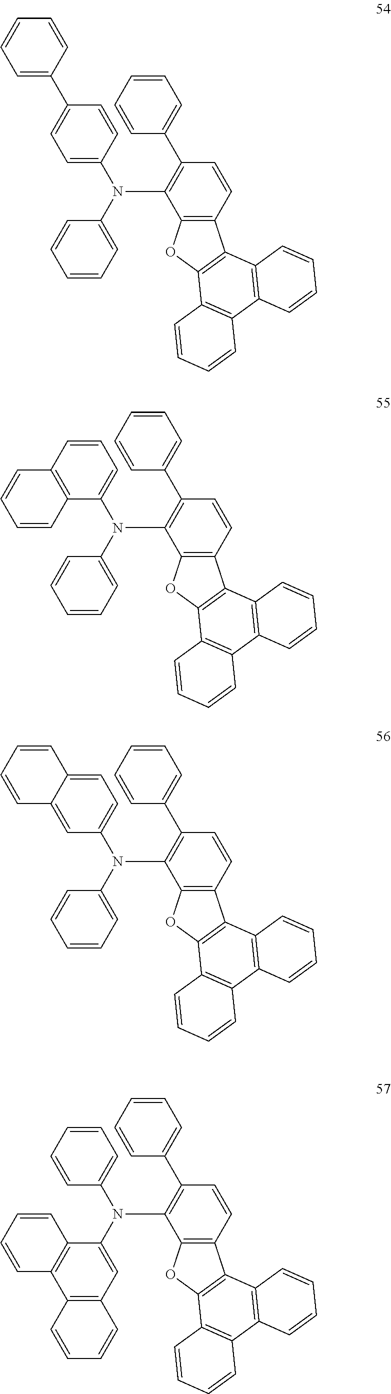 [SMILES: c1ccc(-c2ccc(N(c3ccccc3)c3c(-c4ccccc4)ccc4c3oc3c5ccccc5c5ccccc5c43)cc2)cc1 ; c1ccc(-c2ccc3c(oc4c5ccccc5c5ccccc5c34)c2N(c2ccccc2)c2cccc3ccccc23)cc1 ; c1ccc(-c2ccc3c(oc4c5ccccc5c5ccccc5c34)c2N(c2ccccc2)c2ccc3ccccc3c2)cc1 ; c1ccc(-c2ccc3c(oc4c5ccccc5c5ccccc5c34)c2N(c2ccccc2)c2cc3ccccc3c3ccccc23)cc1]

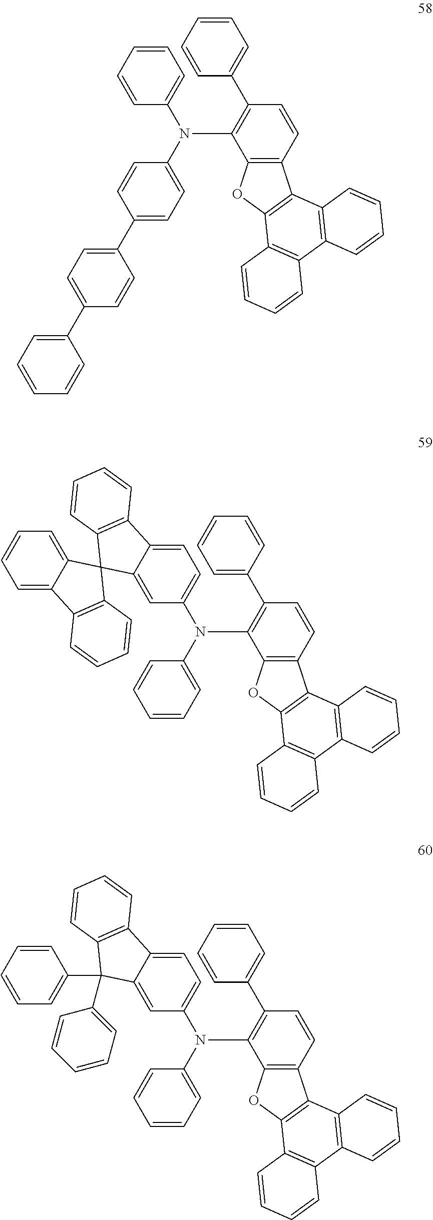 [SMILES: c1ccc(-c2ccc(-c3ccc(N(c4ccccc4)c4c(-c5ccccc5)ccc5c4oc4c6ccccc6c6ccccc6c54)cc3)cc2)cc1 ; c1ccc(-c2ccc3c(oc4c5ccccc5c5ccccc5c34)c2N(c2ccccc2)c2ccc3c(c2)C2(c4ccccc4-c4ccccc42)c2ccccc2-3)cc1 ; c1ccc(-c2ccc3c(oc4c5ccccc5c5ccccc5c34)c2N(c2ccccc2)c2ccc3c(c2)C(c2ccccc2)(c2ccccc2)c2ccccc2-3)cc1]

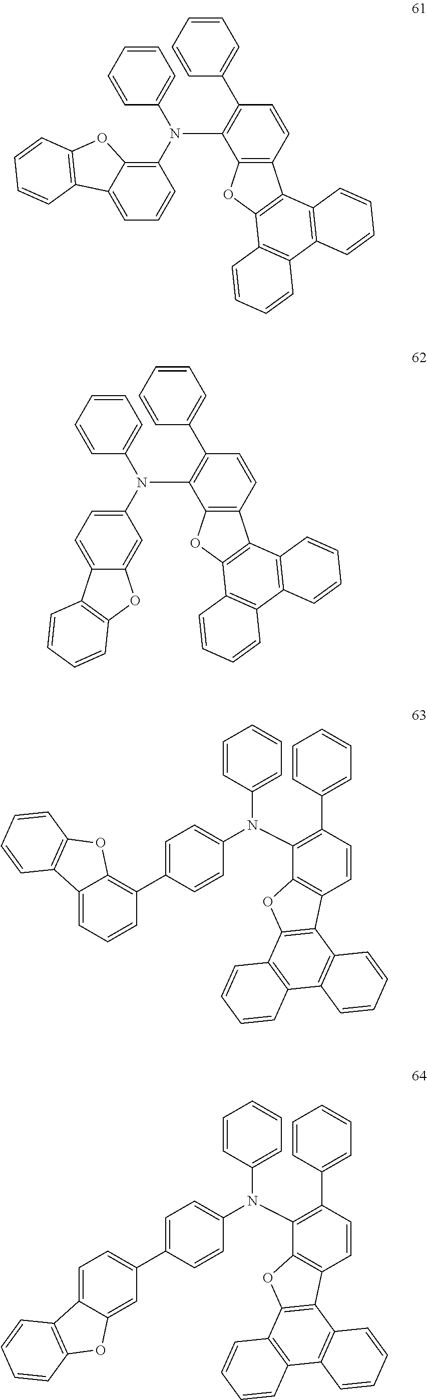 [SMILES: c1ccc(-c2ccc3c(oc4c5ccccc5c5ccccc5c34)c2N(c2ccccc2)c2cccc3c2oc2ccccc23)cc1 ; c1ccc(-c2ccc3c(oc4c5ccccc5c5ccccc5c34)c2N(c2ccccc2)c2ccc3c(c2)oc2ccccc23)cc1 ; c1ccc(-c2ccc3c(oc4c5ccccc5c5ccccc5c34)c2N(c2ccccc2)c2ccc(-c3cccc4c3oc3ccccc34)cc2)cc1 ; c1ccc(-c2ccc3c(oc4c5ccccc5c5ccccc5c34)c2N(c2ccccc2)c2ccc(-c3ccc4c(c3)oc3ccccc34)cc2)cc1]

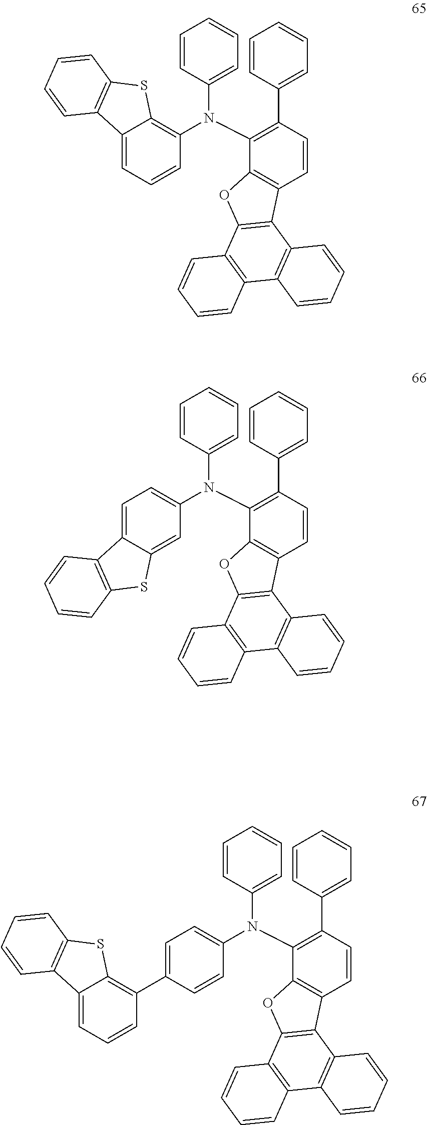 [SMILES: c1ccc(-c2ccc3c(oc4c5ccccc5c5ccccc5c34)c2N(c2ccccc2)c2cccc3c2sc2ccccc23)cc1 ; c1ccc(-c2ccc3c(oc4c5ccccc5c5ccccc5c34)c2N(c2ccccc2)c2ccc3c(c2)sc2ccccc23)cc1 ; c1ccc(-c2ccc3c(oc4c5ccccc5c5ccccc5c34)c2N(c2ccccc2)c2ccc(-c3cccc4c3sc3ccccc34)cc2)cc1]

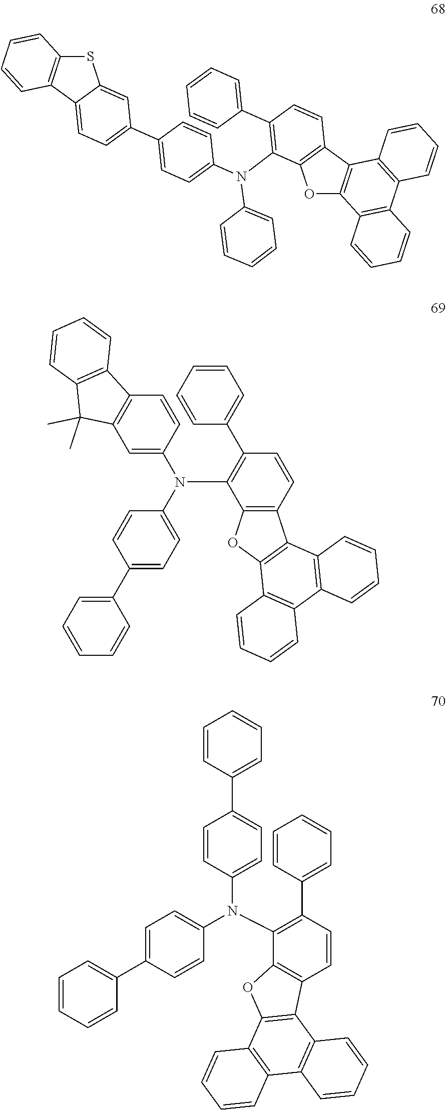 [SMILES: c1ccc(-c2ccc3c(oc4c5ccccc5c5ccccc5c34)c2N(c2ccccc2)c2ccc(-c3ccc4c(c3)sc3ccccc34)cc2)cc1 ; CC1(C)c2ccccc2-c2ccc(N(c3ccc(-c4ccccc4)cc3)c3c(-c4ccccc4)ccc4c3oc3c5ccccc5c5ccccc5c43)cc21 ; c1ccc(-c2ccc(N(c3ccc(-c4ccccc4)cc3)c3c(-c4ccccc4)ccc4c3oc3c5ccccc5c5ccccc5c43)cc2)cc1]

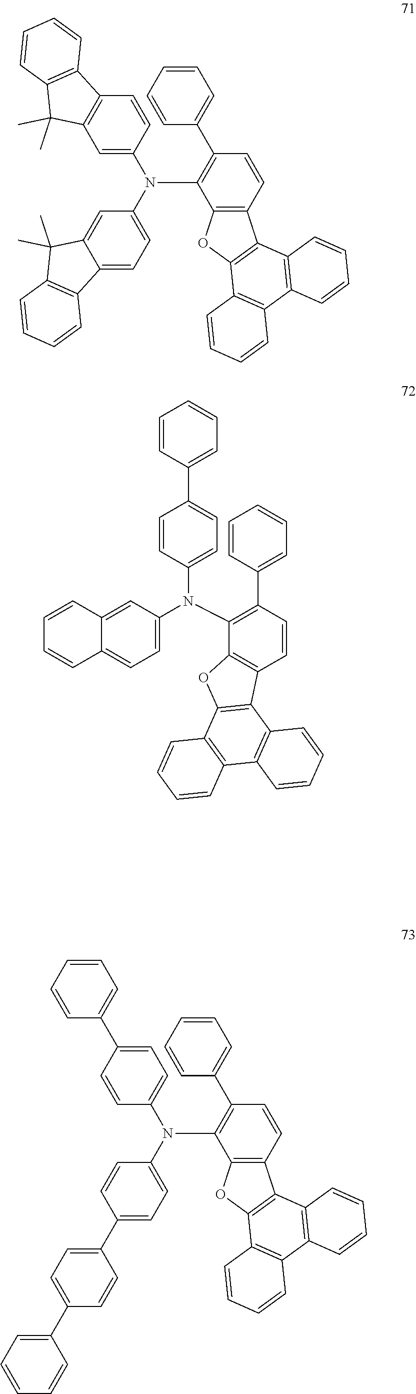 [SMILES: CC1(C)c2ccccc2-c2ccc(N(c3ccc4c(c3)C(C)(C)c3ccccc3-4)c3c(-c4ccccc4)ccc4c3oc3c5ccccc5c5ccccc5c43)cc21 ; c1ccc(-c2ccc(N(c3ccc4ccccc4c3)c3c(-c4ccccc4)ccc4c3oc3c5ccccc5c5ccccc5c43)cc2)cc1 ; c1ccc(-c2ccc(-c3ccc(N(c4ccc(-c5ccccc5)cc4)c4c(-c5ccccc5)ccc5c4oc4c6ccccc6c6ccccc6c54)cc3)cc2)cc1]

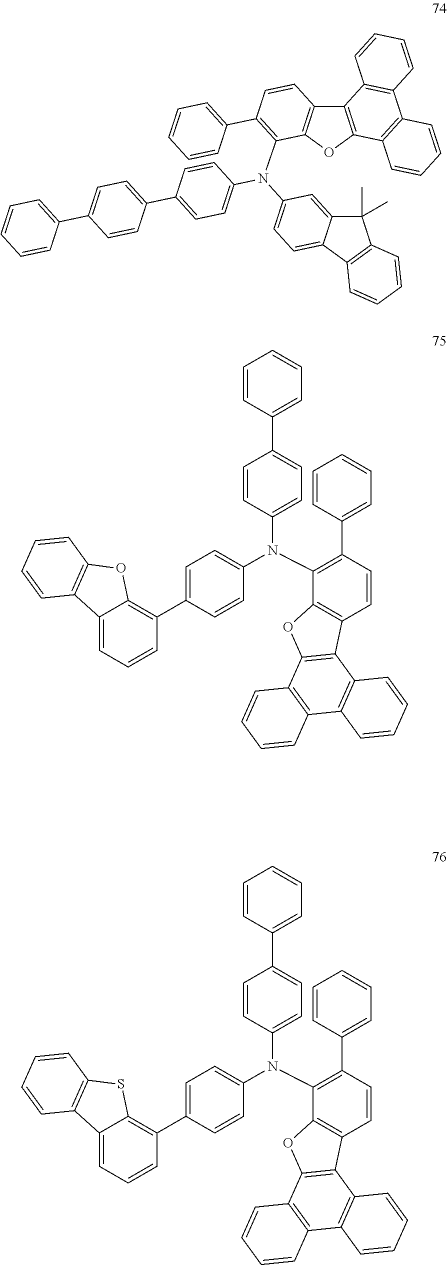 [SMILES: CC1(C)c2ccccc2-c2ccc(N(c3ccc(-c4ccc(-c5ccccc5)cc4)cc3)c3c(-c4ccccc4)ccc4c3oc3c5ccccc5c5ccccc5c43)cc21 ; c1ccc(-c2ccc(N(c3ccc(-c4cccc5c4oc4ccccc45)cc3)c3c(-c4ccccc4)ccc4c3oc3c5ccccc5c5ccccc5c43)cc2)cc1 ; c1ccc(-c2ccc(N(c3ccc(-c4cccc5c4sc4ccccc45)cc3)c3c(-c4ccccc4)ccc4c3oc3c5ccccc5c5ccccc5c43)cc2)cc1]

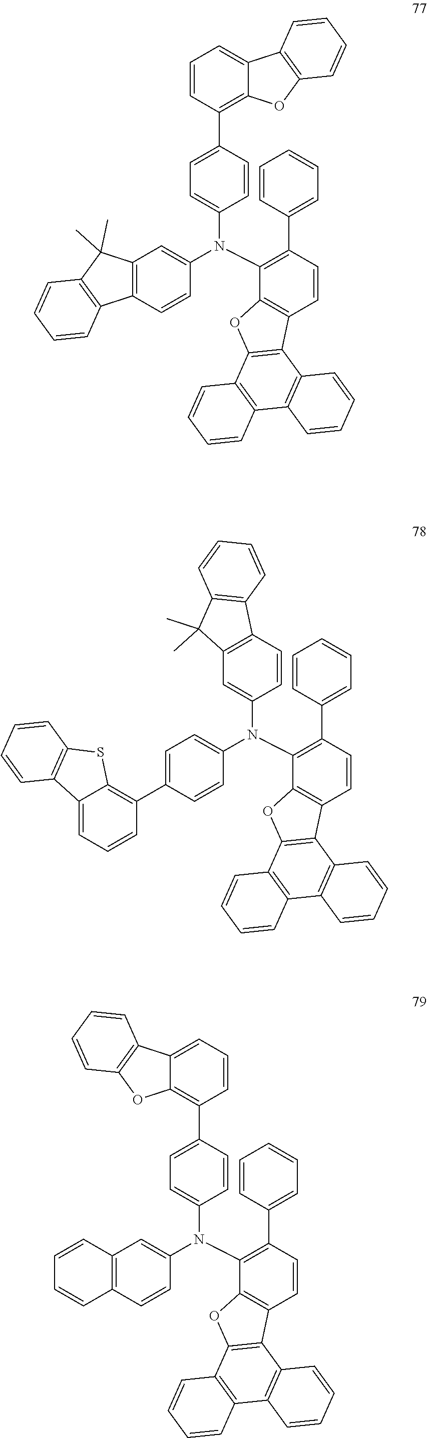 [SMILES: CC1(C)c2ccccc2-c2ccc(N(c3ccc(-c4cccc5c4oc4ccccc45)cc3)c3c(-c4ccccc4)ccc4c3oc3c5ccccc5c5ccccc5c43)cc21 ; CC1(C)c2ccccc2-c2ccc(N(c3ccc(-c4cccc5c4sc4ccccc45)cc3)c3c(-c4ccccc4)ccc4c3oc3c5ccccc5c5ccccc5c43)cc21 ; c1ccc(-c2ccc3c(oc4c5ccccc5c5ccccc5c34)c2N(c2ccc(-c3cccc4c3oc3ccccc34)cc2)c2ccc3ccccc3c2)cc1]

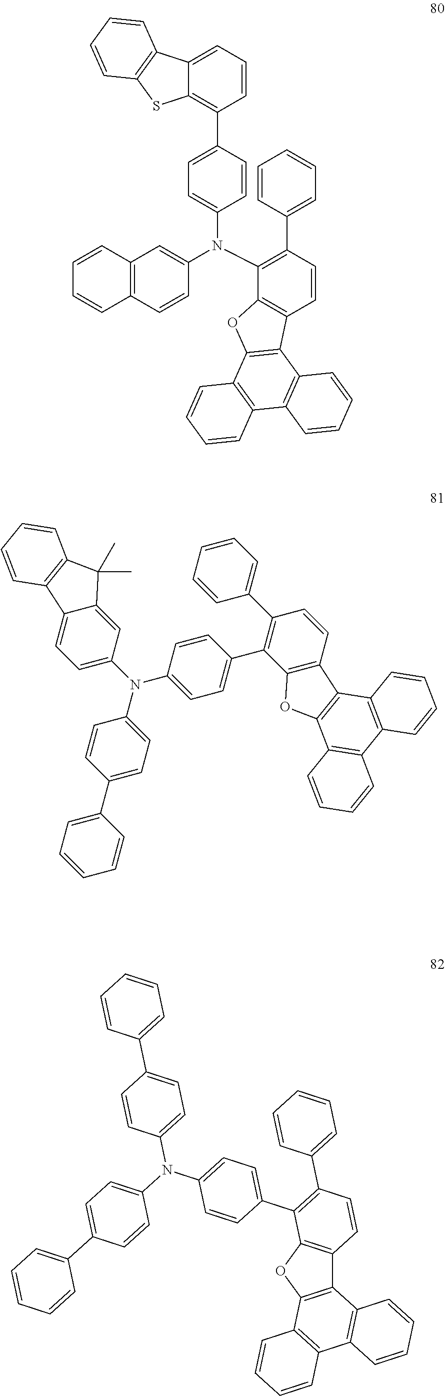 [SMILES: c1ccc(-c2ccc3c(oc4c5ccccc5c5ccccc5c34)c2N(c2ccc(-c3cccc4c3sc3ccccc34)cc2)c2ccc3ccccc3c2)cc1 ; CC1(C)c2ccccc2-c2ccc(N(c3ccc(-c4ccccc4)cc3)c3ccc(-c4c(-c5ccccc5)ccc5c4oc4c6ccccc6c6ccccc6c54)cc3)cc21 ; c1ccc(-c2ccc(N(c3ccc(-c4ccccc4)cc3)c3ccc(-c4c(-c5ccccc5)ccc5c4oc4c6ccccc6c6ccccc6c54)cc3)cc2)cc1]

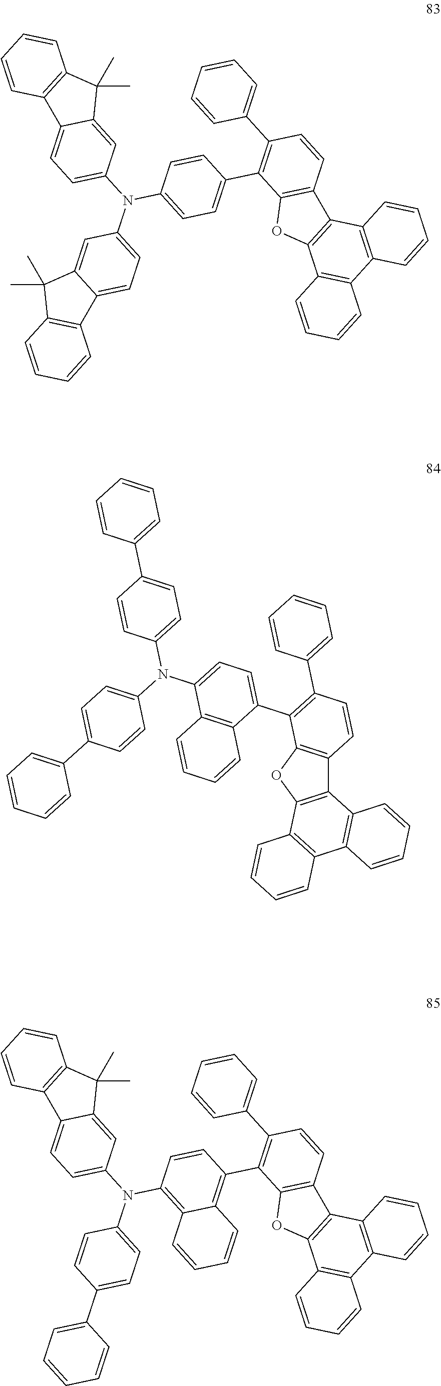 [SMILES: CC1(C)c2ccccc2-c2ccc(N(c3ccc(-c4c(-c5ccccc5)ccc5c4oc4c6ccccc6c6ccccc6c54)cc3)c3ccc4c(c3)C(C)(C)c3ccccc3-4)cc21 ; c1ccc(-c2ccc(N(c3ccc(-c4ccccc4)cc3)c3ccc(-c4c(-c5ccccc5)ccc5c4oc4c6ccccc6c6ccccc6c54)c4ccccc34)cc2)cc1 ; CC1(C)c2ccccc2-c2ccc(N(c3ccc(-c4ccccc4)cc3)c3ccc(-c4c(-c5ccccc5)ccc5c4oc4c6ccccc6c6ccccc6c54)c4ccccc34)cc21]

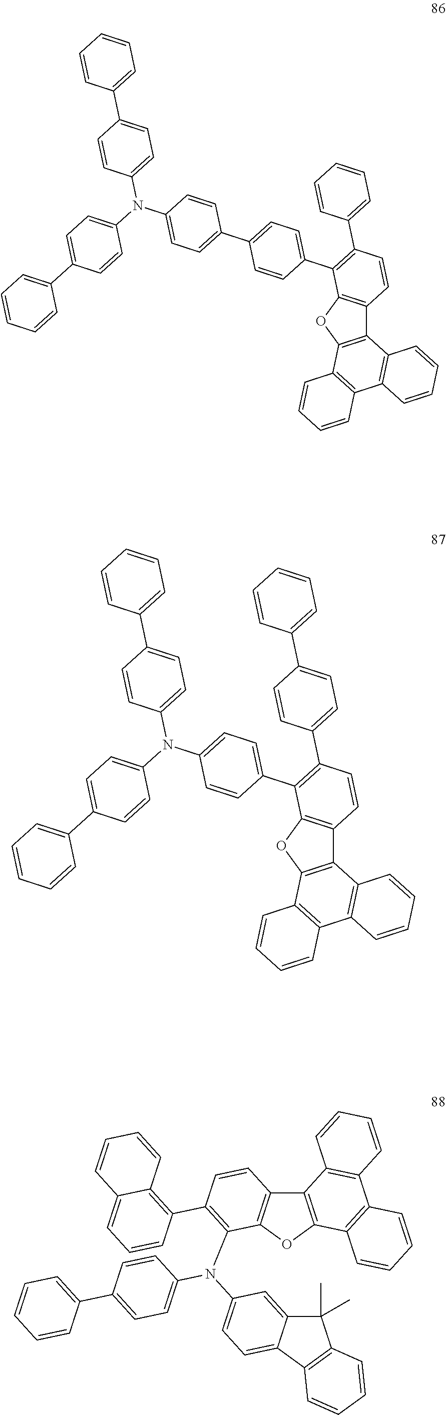 [SMILES: c1ccc(-c2ccc(N(c3ccc(-c4ccccc4)cc3)c3ccc(-c4ccc(-c5c(-c6ccccc6)ccc6c5oc5c7ccccc7c7ccccc7c65)cc4)cc3)cc2)cc1 ; c1ccc(-c2ccc(-c3ccc4c(oc5c6ccccc6c6ccccc6c45)c3-c3ccc(N(c4ccc(-c5ccccc5)cc4)c4ccc(-c5ccccc5)cc4)cc3)cc2)cc1 ; CC1(C)c2ccccc2-c2ccc(N(c3ccc(-c4ccccc4)cc3)c3c(-c4cccc5ccccc45)ccc4c3oc3c5ccccc5c5ccccc5c43)cc21]

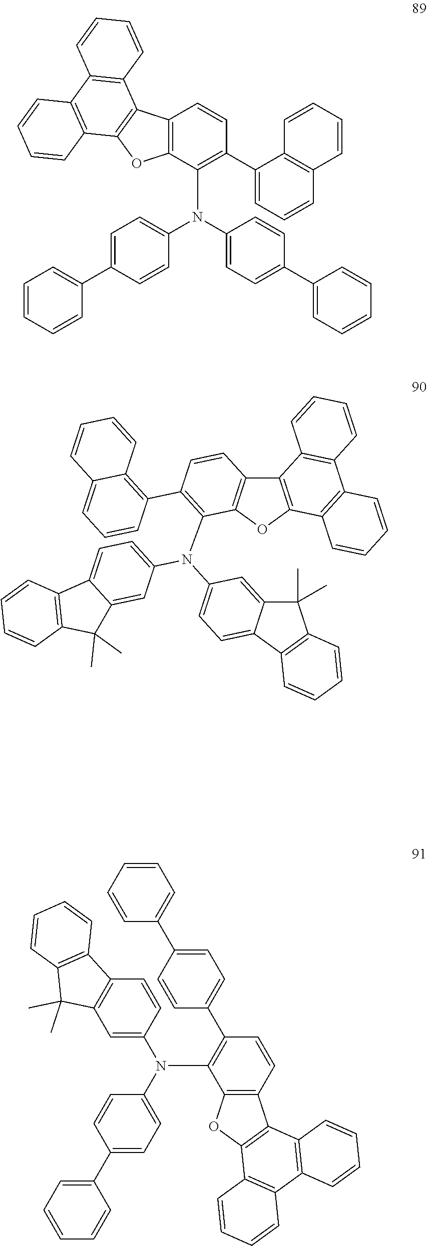 [SMILES: c1ccc(-c2ccc(N(c3ccc(-c4ccccc4)cc3)c3c(-c4cccc5ccccc45)ccc4c3oc3c5ccccc5c5ccccc5c43)cc2)cc1 ; CC1(C)c2ccccc2-c2ccc(N(c3ccc4c(c3)C(C)(C)c3ccccc3-4)c3c(-c4cccc5ccccc45)ccc4c3oc3c5ccccc5c5ccccc5c43)cc21 ; CC1(C)c2ccccc2-c2ccc(N(c3ccc(-c4ccccc4)cc3)c3c(-c4ccc(-c5ccccc5)cc4)ccc4c3oc3c5ccccc5c5ccccc5c43)cc21]

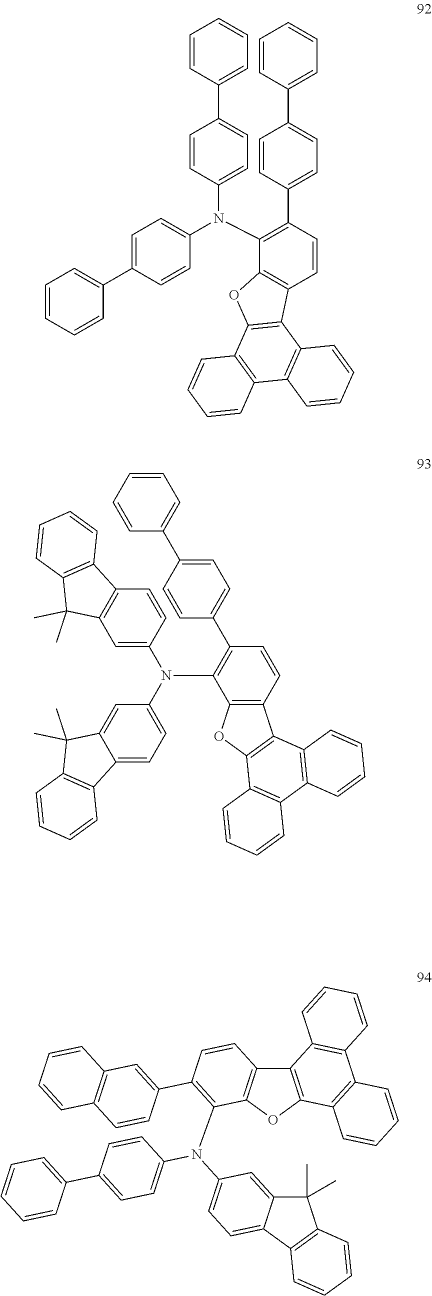 [SMILES: c1ccc(-c2ccc(-c3ccc4c(oc5c6ccccc6c6ccccc6c45)c3N(c3ccc(-c4ccccc4)cc3)c3ccc(-c4ccccc4)cc3)cc2)cc1 ; CC1(C)c2ccccc2-c2ccc(N(c3ccc4c(c3)C(C)(C)c3ccccc3-4)c3c(-c4ccc(-c5ccccc5)cc4)ccc4c3oc3c5ccccc5c5ccccc5c43)cc21 ; CC1(C)c2ccccc2-c2ccc(N(c3ccc(-c4ccccc4)cc3)c3c(-c4ccc5ccccc5c4)ccc4c3oc3c5ccccc5c5ccccc5c43)cc21]

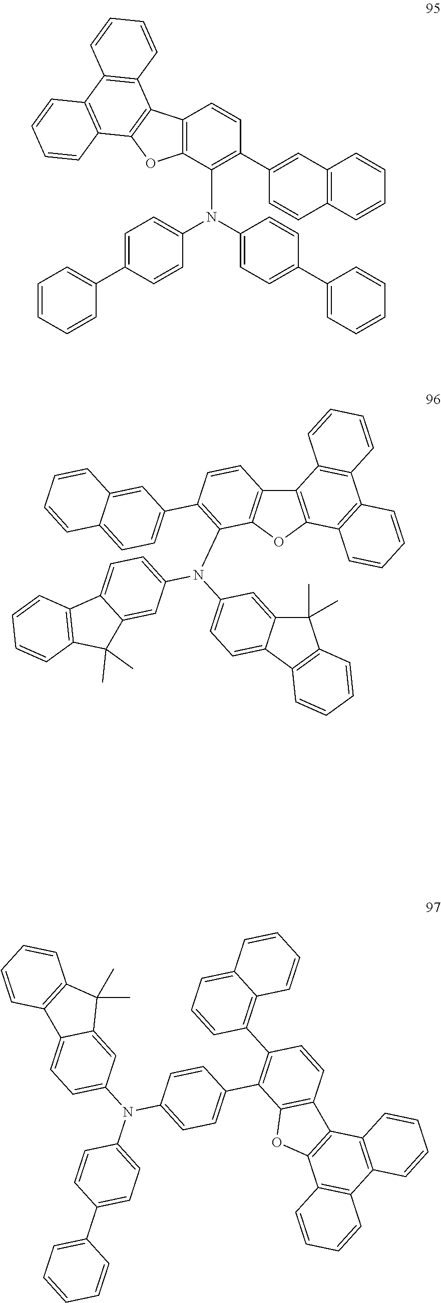 [SMILES: c1ccc(-c2ccc(N(c3ccc(-c4ccccc4)cc3)c3c(-c4ccc5ccccc5c4)ccc4c3oc3c5ccccc5c5ccccc5c43)cc2)cc1 ; CC1(C)c2ccccc2-c2ccc(N(c3ccc4c(c3)C(C)(C)c3ccccc3-4)c3c(-c4ccc5ccccc5c4)ccc4c3oc3c5ccccc5c5ccccc5c43)cc21 ; CC1(C)c2ccccc2-c2ccc(N(c3ccc(-c4ccccc4)cc3)c3ccc(-c4c(-c5cccc6ccccc56)ccc5c4oc4c6ccccc6c6ccccc6c54)cc3)cc21]

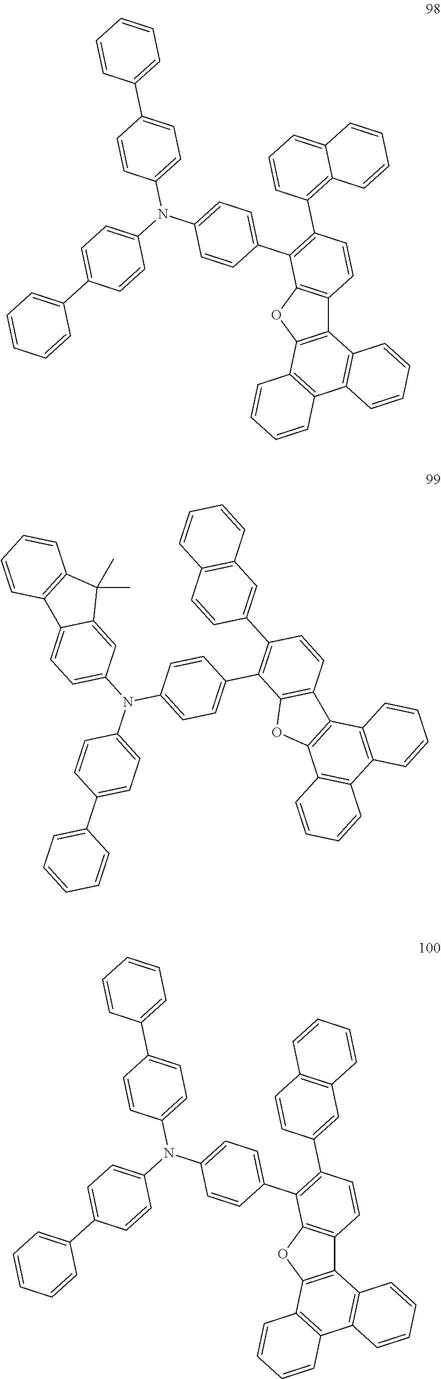 [SMILES: c1ccc(-c2ccc(N(c3ccc(-c4ccccc4)cc3)c3ccc(-c4c(-c5cccc6ccccc56)ccc5c4oc4c6ccccc6c6ccccc6c54)cc3)cc2)cc1 ; CC1(C)c2ccccc2-c2ccc(N(c3ccc(-c4ccccc4)cc3)c3ccc(-c4c(-c5ccc6ccccc6c5)ccc5c4oc4c6ccccc6c6ccccc6c54)cc3)cc21 ; c1ccc(-c2ccc(N(c3ccc(-c4ccccc4)cc3)c3ccc(-c4c(-c5ccc6ccccc6c5)ccc5c4oc4c6ccccc6c6ccccc6c54)cc3)cc2)cc1]

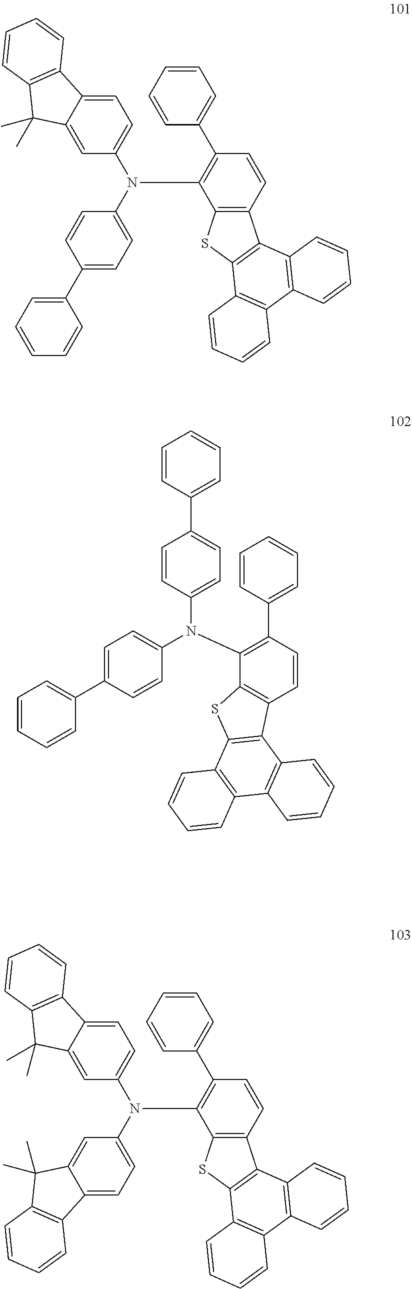 [SMILES: CC1(C)c2ccccc2-c2ccc(N(c3ccc(-c4ccccc4)cc3)c3c(-c4ccccc4)ccc4c3sc3c5ccccc5c5ccccc5c43)cc21 ; c1ccc(-c2ccc(N(c3ccc(-c4ccccc4)cc3)c3c(-c4ccccc4)ccc4c3sc3c5ccccc5c5ccccc5c43)cc2)cc1 ; CC1(C)c2ccccc2-c2ccc(N(c3ccc4c(c3)C(C)(C)c3ccccc3-4)c3c(-c4ccccc4)ccc4c3sc3c5ccccc5c5ccccc5c43)cc21]

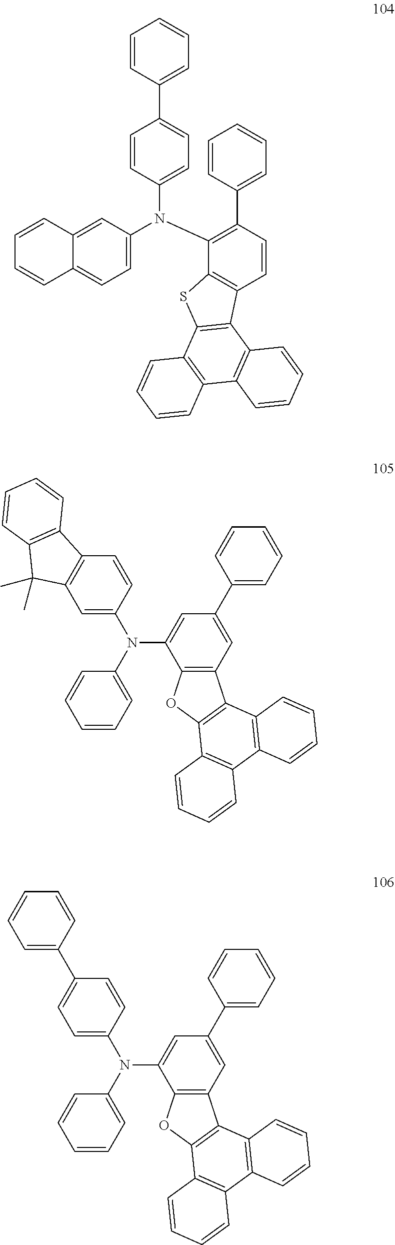 [SMILES: c1ccc(-c2ccc(N(c3ccc4ccccc4c3)c3c(-c4ccccc4)ccc4c3sc3c5ccccc5c5ccccc5c43)cc2)cc1 ; CC1(C)c2ccccc2-c2ccc(N(c3ccccc3)c3cc(-c4ccccc4)cc4c3oc3c5ccccc5c5ccccc5c43)cc21 ; c1ccc(-c2ccc(N(c3ccccc3)c3cc(-c4ccccc4)cc4c3oc3c5ccccc5c5ccccc5c43)cc2)cc1]

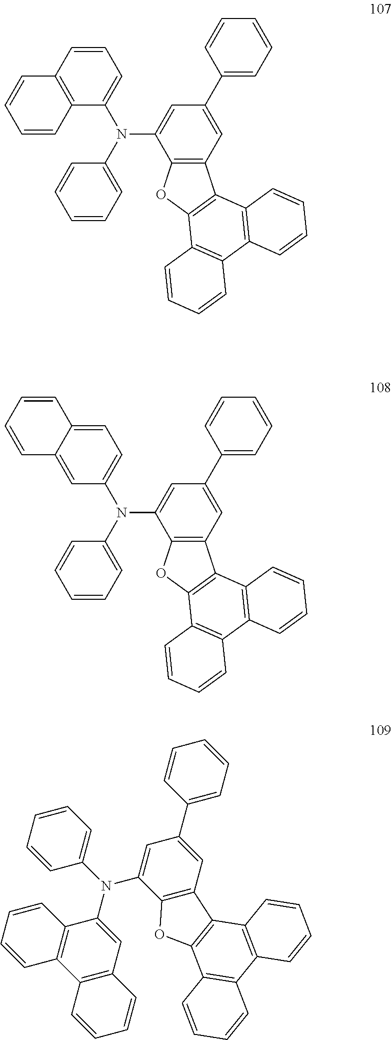 [SMILES: c1ccc(-c2cc(N(c3ccccc3)c3cccc4ccccc34)c3oc4c5ccccc5c5ccccc5c4c3c2)cc1 ; c1ccc(-c2cc(N(c3ccccc3)c3ccc4ccccc4c3)c3oc4c5ccccc5c5ccccc5c4c3c2)cc1 ; c1ccc(-c2cc(N(c3ccccc3)c3cc4ccccc4c4ccccc34)c3oc4c5ccccc5c5ccccc5c4c3c2)cc1]

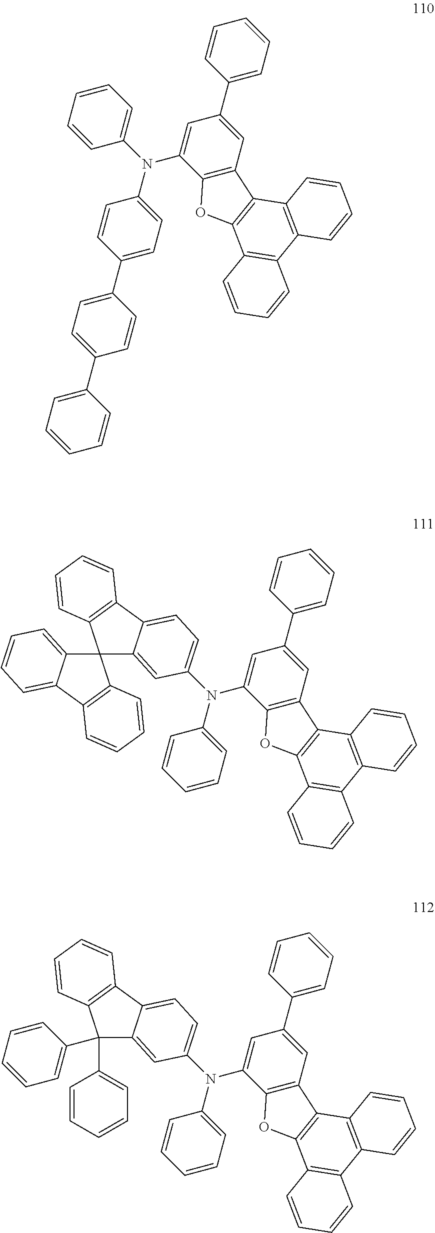 [SMILES: c1ccc(-c2ccc(-c3ccc(N(c4ccccc4)c4cc(-c5ccccc5)cc5c4oc4c6ccccc6c6ccccc6c54)cc3)cc2)cc1 ; c1ccc(-c2cc(N(c3ccccc3)c3ccc4c(c3)C3(c5ccccc5-c5ccccc53)c3ccccc3-4)c3oc4c5ccccc5c5ccccc5c4c3c2)cc1 ; c1ccc(-c2cc(N(c3ccccc3)c3ccc4c(c3)C(c3ccccc3)(c3ccccc3)c3ccccc3-4)c3oc4c5ccccc5c5ccccc5c4c3c2)cc1]

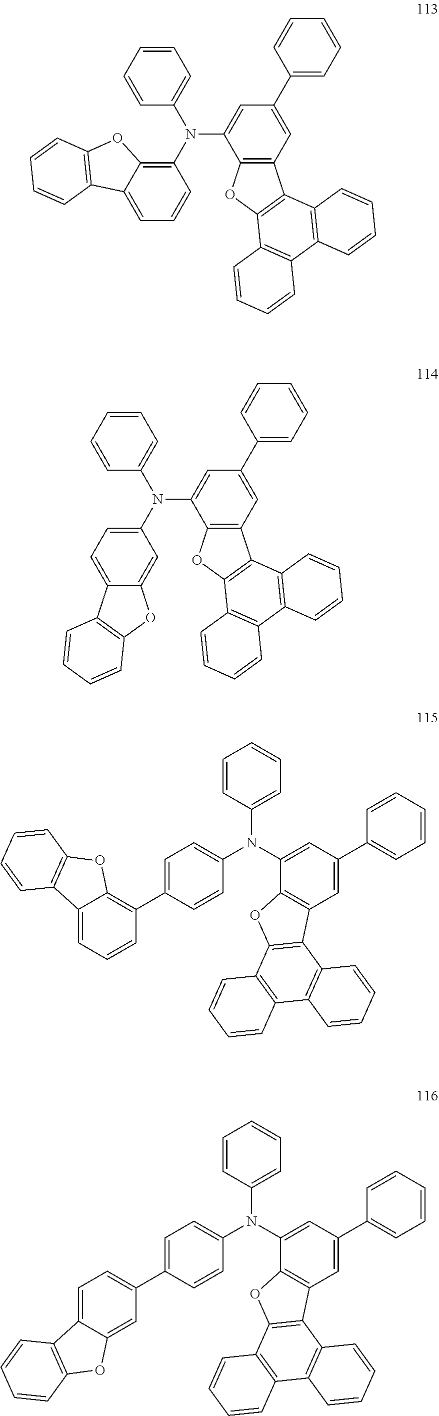 [SMILES: c1ccc(-c2cc(N(c3ccccc3)c3cccc4c3oc3ccccc34)c3oc4c5ccccc5c5ccccc5c4c3c2)cc1 ; c1ccc(-c2cc(N(c3ccccc3)c3ccc4c(c3)oc3ccccc34)c3oc4c5ccccc5c5ccccc5c4c3c2)cc1 ; c1ccc(-c2cc(N(c3ccccc3)c3ccc(-c4cccc5c4oc4ccccc45)cc3)c3oc4c5ccccc5c5ccccc5c4c3c2)cc1 ; c1ccc(-c2cc(N(c3ccccc3)c3ccc(-c4ccc5c(c4)oc4ccccc45)cc3)c3oc4c5ccccc5c5ccccc5c4c3c2)cc1]

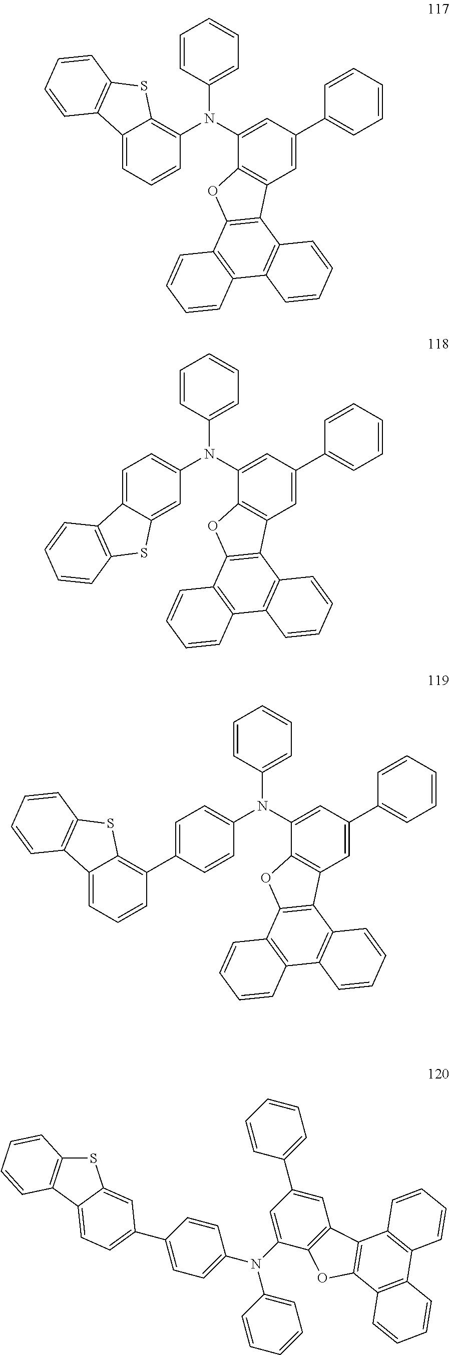

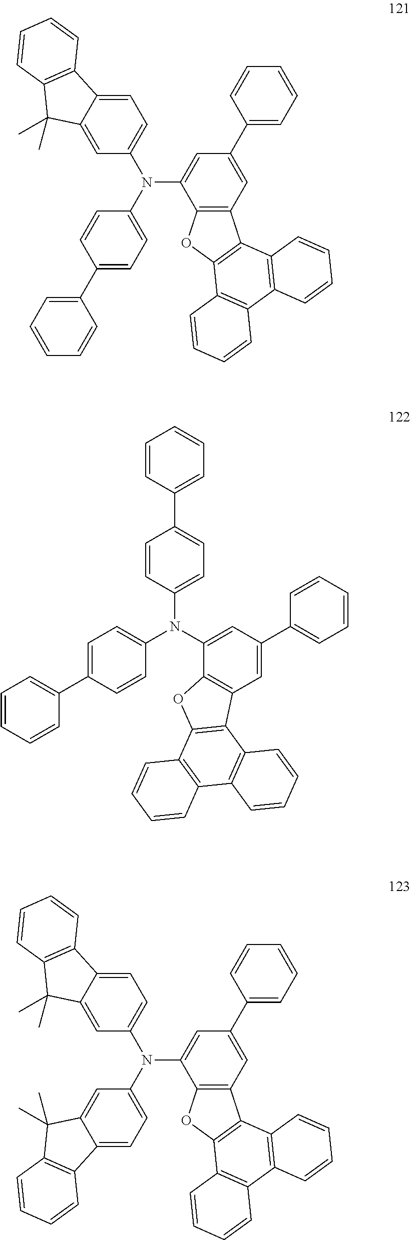 [SMILES: CC1(C)c2ccccc2-c2ccc(N(c3ccc(-c4ccccc4)cc3)c3cc(-c4ccccc4)cc4c3oc3c5ccccc5c5ccccc5c43)cc21 ; c1ccc(-c2ccc(N(c3ccc(-c4ccccc4)cc3)c3cc(-c4ccccc4)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4ccccc4)cc4c3oc3c5ccccc5c5ccccc5c43)cc21]

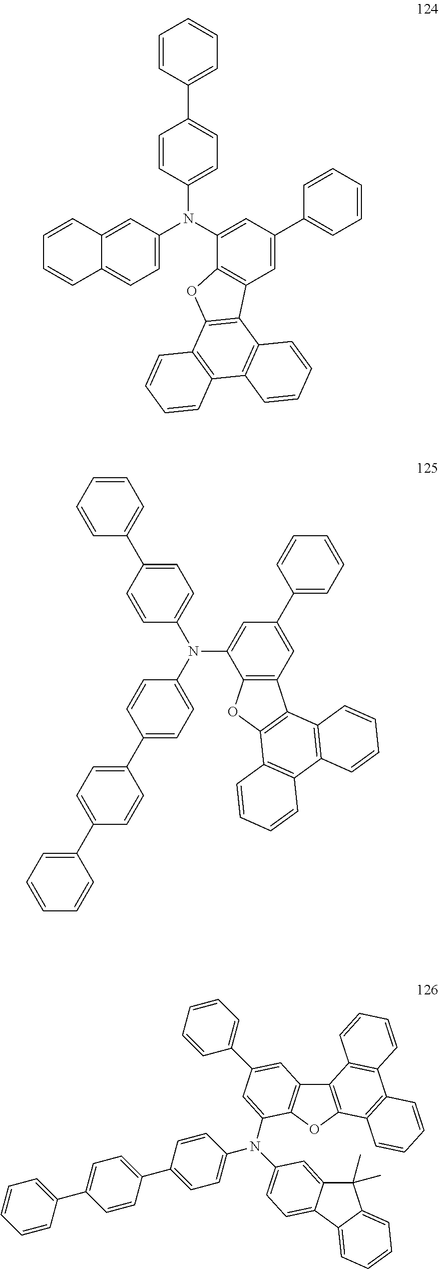 [SMILES: c1ccc(-c2ccc(N(c3ccc4ccccc4c3)c3cc(-c4ccccc4)cc4c3oc3c5ccccc5c5ccccc5c43)cc2)cc1 ; c1ccc(-c2ccc(-c3ccc(N(c4ccc(-c5ccccc5)cc4)c4cc(-c5ccccc5)cc5c4oc4c6ccccc6c6ccccc6c54)cc3)cc2)cc1 ; CC1(C)c2ccccc2-c2ccc(N(c3ccc(-c4ccc(-c5ccccc5)cc4)cc3)c3cc(-c4ccccc4)cc4c3oc3c5ccccc5c5ccccc5c43)cc21]

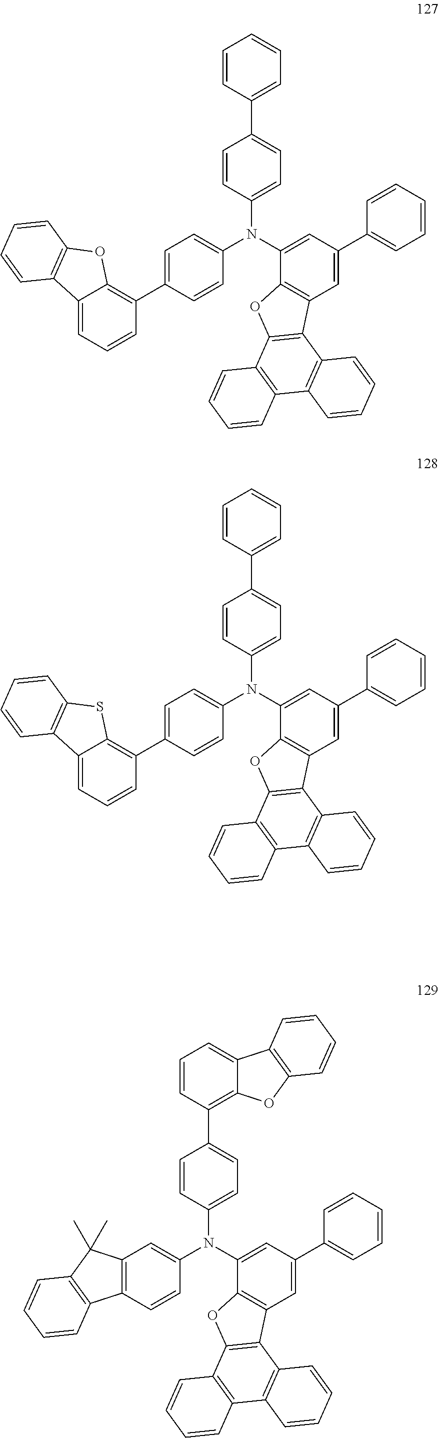 [SMILES: c1ccc(-c2ccc(N(c3ccc(-c4cccc5c4oc4ccccc45)cc3)c3cc(-c4ccccc4)cc4c3oc3c5ccccc5c5ccccc5c43)cc2)cc1 ; c1ccc(-c2ccc(N(c3ccc(-c4cccc5c4sc4ccccc45)cc3)c3cc(-c4ccccc4)cc4c3oc3c5ccccc5c5ccccc5c43)cc2)cc1 ; CC1(C)c2ccccc2-c2ccc(N(c3ccc(-c4cccc5c4oc4ccccc45)cc3)c3cc(-c4ccccc4)cc4c3oc3c5ccccc5c5ccccc5c43)cc21]

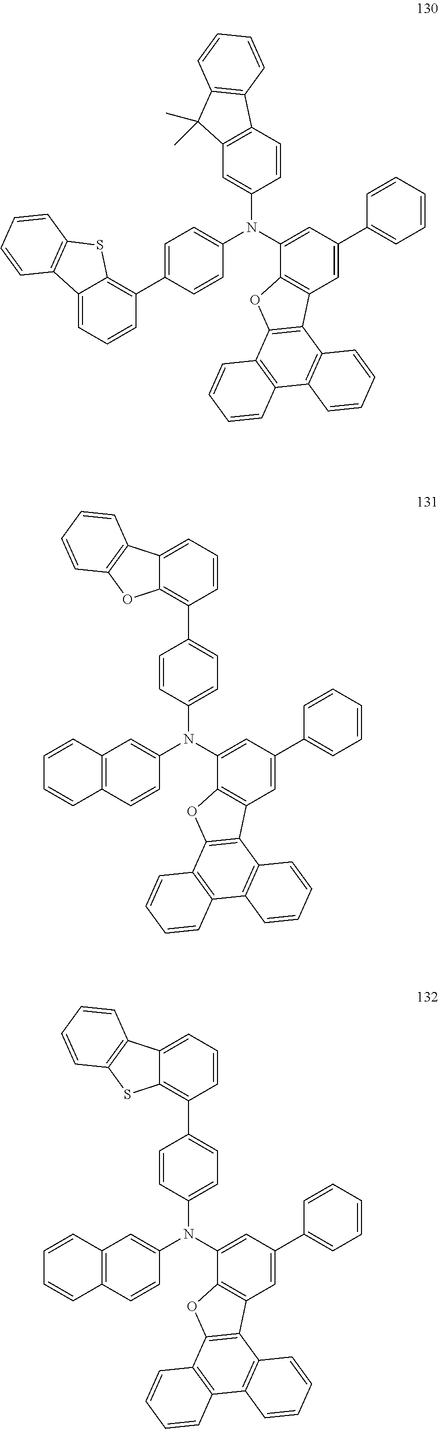 [SMILES: CC1(C)c2ccccc2-c2ccc(N(c3ccc(-c4cccc5c4sc4ccccc45)cc3)c3cc(-c4ccccc4)cc4c3oc3c5ccccc5c5ccccc5c43)cc21 ; c1ccc(-c2cc(N(c3ccc(-c4cccc5c4oc4ccccc45)cc3)c3ccc4ccccc4c3)c3oc4c5ccccc5c5ccccc5c4c3c2)cc1 ; c1ccc(-c2cc(N(c3ccc(-c4cccc5c4sc4ccccc45)cc3)c3ccc4ccccc4c3)c3oc4c5ccccc5c5ccccc5c4c3c2)cc1]

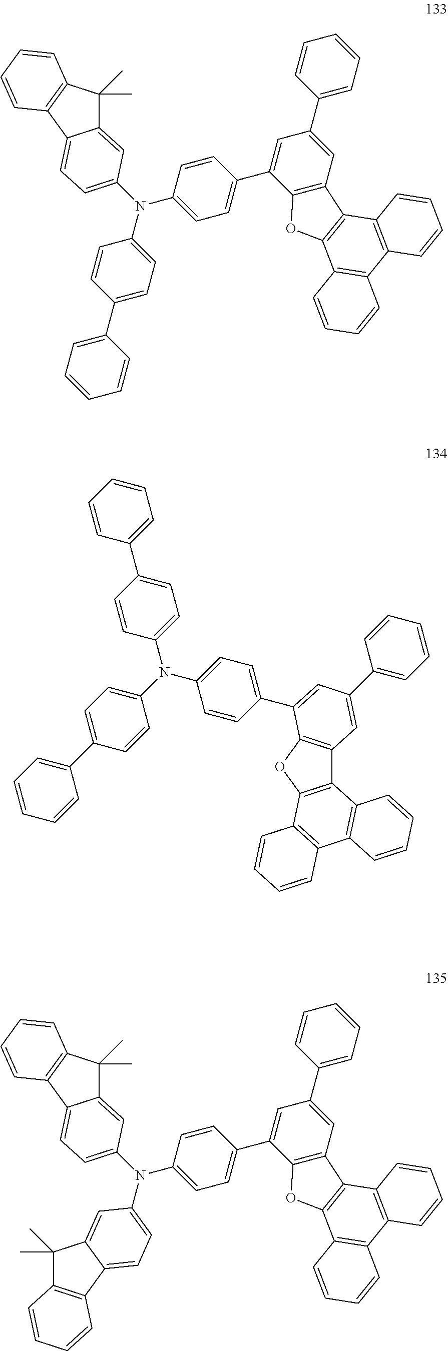 [SMILES: CC1(C)c2ccccc2-c2ccc(N(c3ccc(-c4ccccc4)cc3)c3ccc(-c4cc(-c5ccccc5)cc5c4oc4c6ccccc6c6ccccc6c54)cc3)cc21 ; c1ccc(-c2ccc(N(c3ccc(-c4ccccc4)cc3)c3ccc(-c4cc(-c5ccccc5)cc5c4oc4c6ccccc6c6ccccc6c54)cc3)cc2)cc1 ; CC1(C)c2ccccc2-c2ccc(N(c3ccc(-c4cc(-c5ccccc5)cc5c4oc4c6ccccc6c6ccccc6c54)cc3)c3ccc4c(c3)C(C)(C)c3ccccc3-4)cc21]

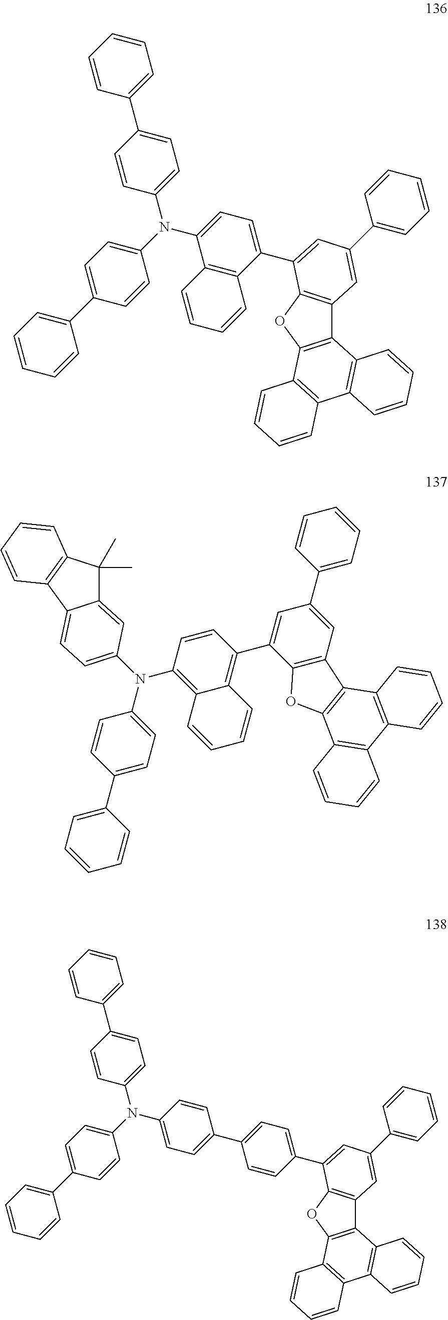 [SMILES: c1ccc(-c2ccc(N(c3ccc(-c4ccccc4)cc3)c3ccc(-c4cc(-c5ccccc5)cc5c4oc4c6ccccc6c6ccccc6c54)c4ccccc34)cc2)cc1 ; CC1(C)c2ccccc2-c2ccc(N(c3ccc(-c4ccccc4)cc3)c3ccc(-c4cc(-c5ccccc5)cc5c4oc4c6ccccc6c6ccccc6c54)c4ccccc34)cc21 ; c1ccc(-c2ccc(N(c3ccc(-c4ccccc4)cc3)c3ccc(-c4ccc(-c5cc(-c6ccccc6)cc6c5oc5c7ccccc7c7ccccc7c65)cc4)cc3)cc2)cc1]

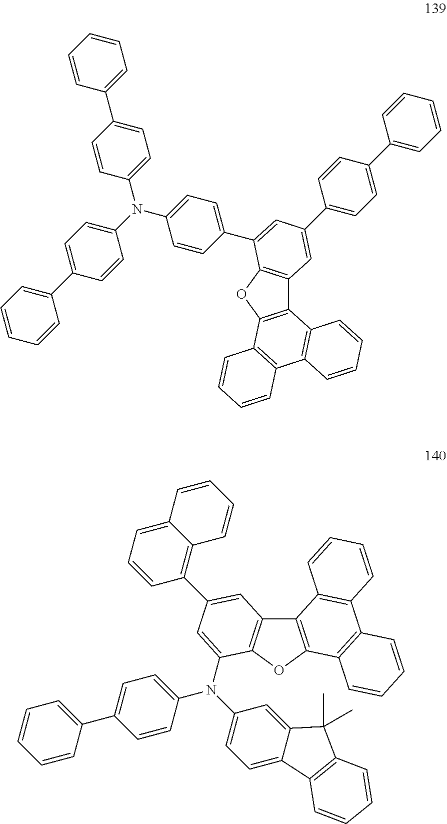 [SMILES: c1ccc(-c2ccc(-c3cc(-c4ccc(N(c5ccc(-c6ccccc6)cc5)c5ccc(-c6ccccc6)cc5)cc4)c4oc5c6ccccc6c6ccccc6c5c4c3)cc2)cc1 ; CC1(C)c2ccccc2-c2ccc(N(c3ccc(-c4ccccc4)cc3)c3cc(-c4cccc5ccccc45)cc4c3oc3c5ccccc5c5ccccc5c43)cc21]

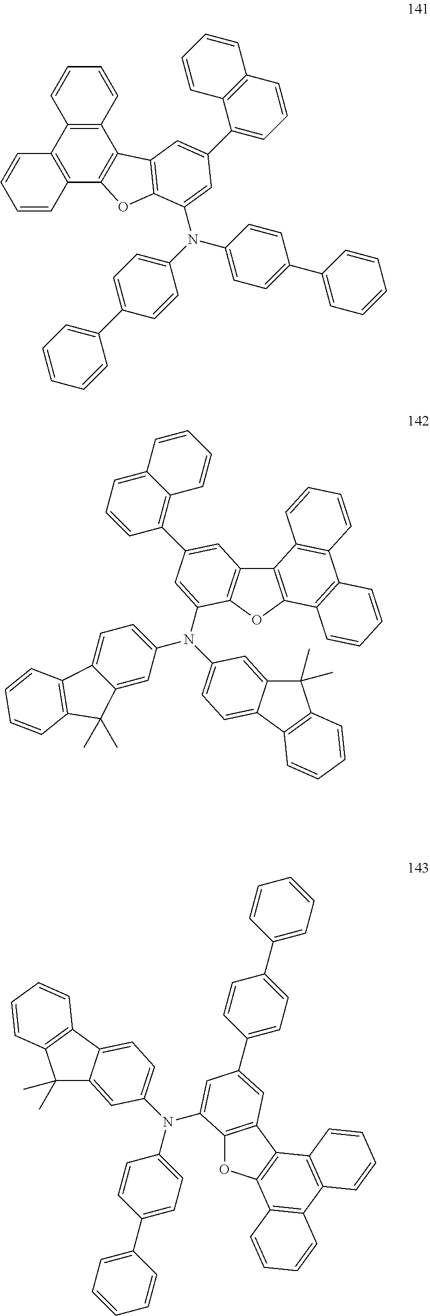 [SMILES: c1ccc(-c2ccc(N(c3ccc(-c4ccccc4)cc3)c3cc(-c4cccc5ccccc45)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4cccc5ccccc45)cc4c3oc3c5ccccc5c5ccccc5c43)cc21 ; CC1(C)c2ccccc2-c2ccc(N(c3ccc(-c4ccccc4)cc3)c3cc(-c4ccc(-c5ccccc5)cc4)cc4c3oc3c5ccccc5c5ccccc5c43)cc21]

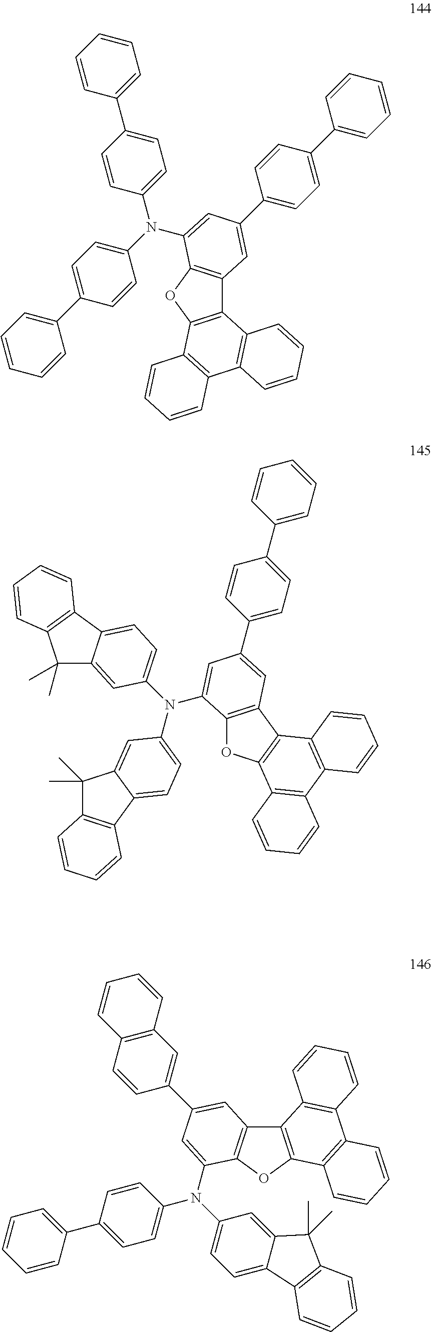 [SMILES: c1ccc(-c2ccc(-c3cc(N(c4ccc(-c5ccccc5)cc4)c4ccc(-c5ccccc5)cc4)c4oc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccc(-c5ccccc5)cc4)cc4c3oc3c5ccccc5c5ccccc5c43)cc21 ; CC1(C)c2ccccc2-c2ccc(N(c3ccc(-c4ccccc4)cc3)c3cc(-c4ccc5ccccc5c4)cc4c3oc3c5ccccc5c5ccccc5c43)cc21]

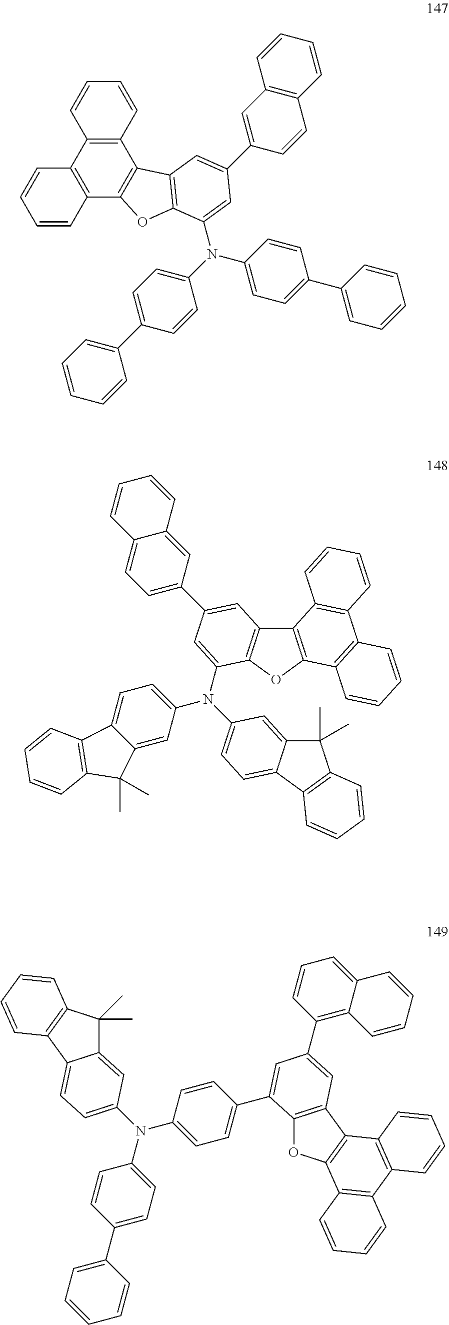 [SMILES: c1ccc(-c2ccc(N(c3ccc(-c4ccccc4)cc3)c3cc(-c4ccc5ccccc5c4)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4ccc5ccccc5c4)cc4c3oc3c5ccccc5c5ccccc5c43)cc21 ; CC1(C)c2ccccc2-c2ccc(N(c3ccc(-c4ccccc4)cc3)c3ccc(-c4cc(-c5cccc6ccccc56)cc5c4oc4c6ccccc6c6ccccc6c54)cc3)cc21]

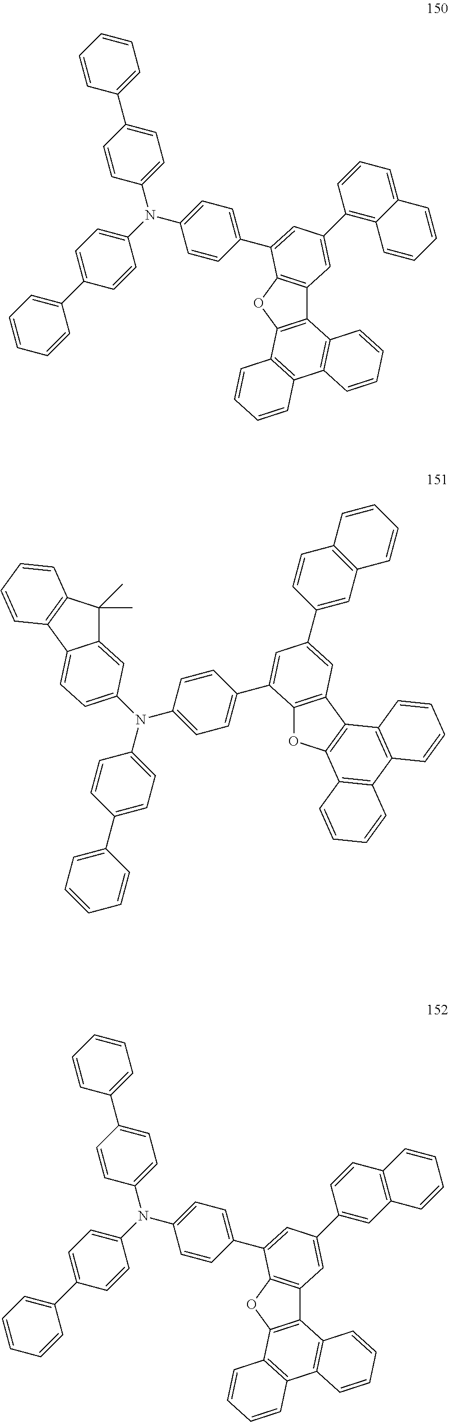 [SMILES: c1ccc(-c2ccc(N(c3ccc(-c4ccccc4)cc3)c3ccc(-c4cc(-c5cccc6ccccc56)cc5c4oc4c6ccccc6c6ccccc6c54)cc3)cc2)cc1 ; CC1(C)c2ccccc2-c2ccc(N(c3ccc(-c4ccccc4)cc3)c3ccc(-c4cc(-c5ccc6ccccc6c5)cc5c4oc4c6ccccc6c6ccccc6c54)cc3)cc21 ; c1ccc(-c2ccc(N(c3ccc(-c4ccccc4)cc3)c3ccc(-c4cc(-c5ccc6ccccc6c5)cc5c4oc4c6ccccc6c6ccccc6c54)cc3)cc2)cc1]

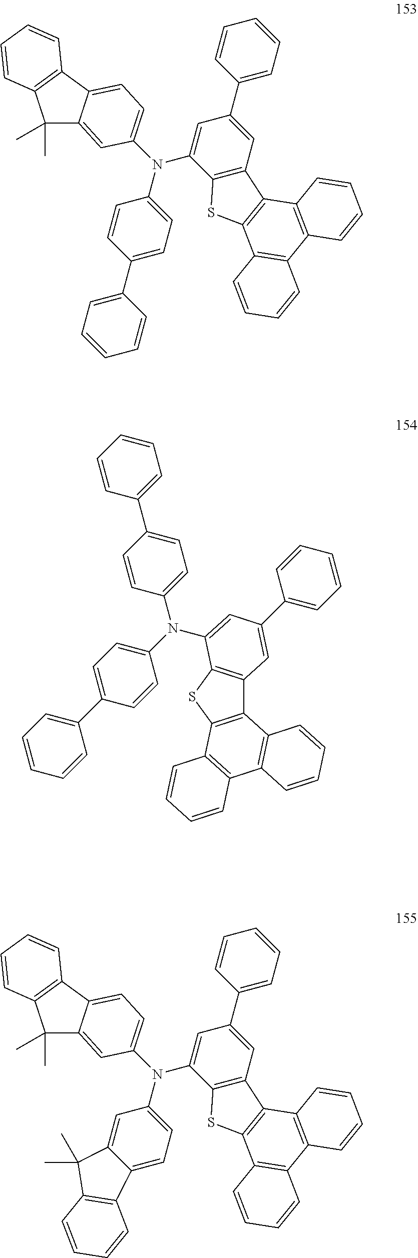 [SMILES: CC1(C)c2ccccc2-c2ccc(N(c3ccc(-c4ccccc4)cc3)c3cc(-c4ccccc4)cc4c3sc3c5ccccc5c5ccccc5c43)cc21 ; c1ccc(-c2ccc(N(c3ccc(-c4ccccc4)cc3)c3cc(-c4ccccc4)cc4c3sc3c5ccccc5c5ccccc5c43)cc2)cc1 ; CC1(C)c2ccccc2-c2ccc(N(c3ccc4c(c3)C(C)(C)c3ccccc3-4)c3cc(-c4ccccc4)cc4c3sc3c5ccccc5c5ccccc5c43)cc21]

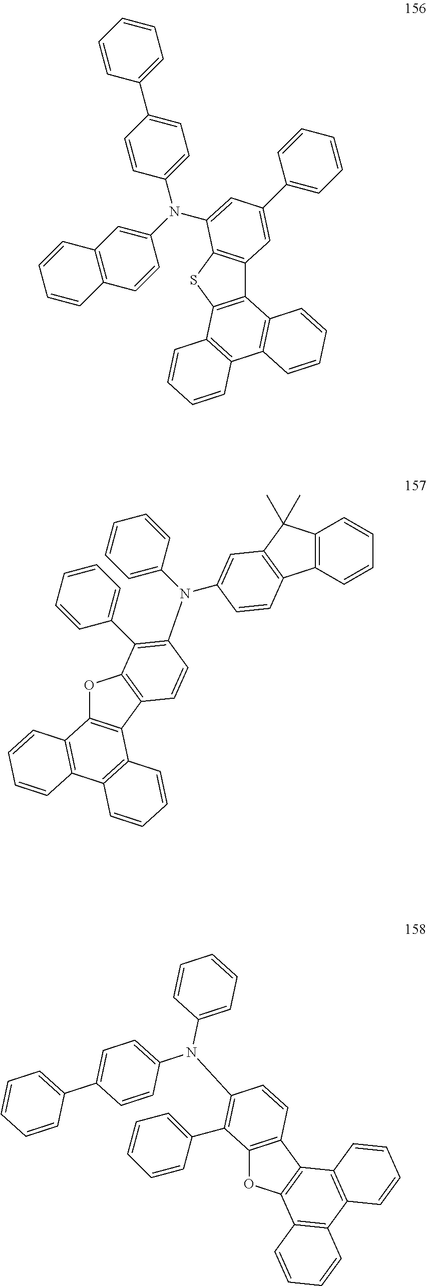 [SMILES: c1ccc(-c2ccc(N(c3ccc4ccccc4c3)c3cc(-c4ccccc4)cc4c3sc3c5ccccc5c5ccccc5c43)cc2)cc1 ; CC1(C)c2ccccc2-c2ccc(N(c3ccccc3)c3ccc4c(oc5c6ccccc6c6ccccc6c45)c3-c3ccccc3)cc21 ; c1ccc(-c2ccc(N(c3ccccc3)c3ccc4c(oc5c6ccccc6c6ccccc6c45)c3-c3ccccc3)cc2)cc1]

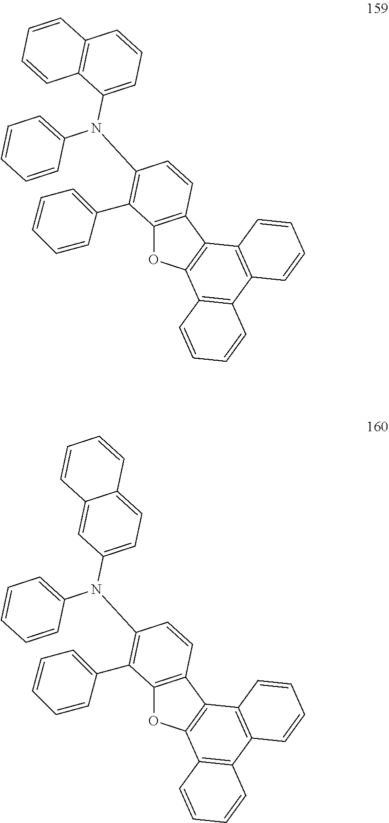 [SMILES: c1ccc(-c2c(N(c3ccccc3)c3cccc4ccccc34)ccc3c2oc2c4ccccc4c4ccccc4c32)cc1 ; c1ccc(-c2c(N(c3ccccc3)c3ccc4ccccc4c3)ccc3c2oc2c4ccccc4c4ccccc4c32)cc1]

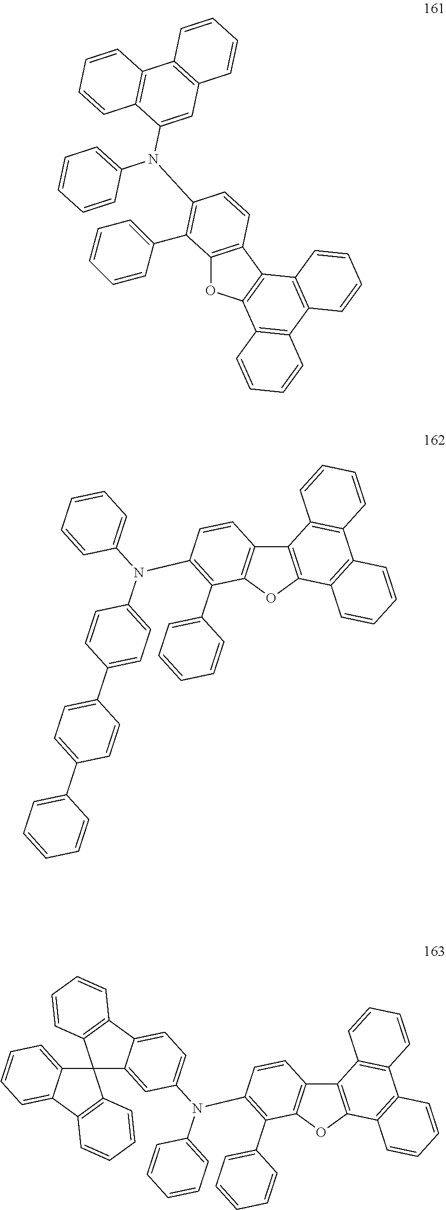 [SMILES: c1ccc(-c2c(N(c3ccccc3)c3cc4ccccc4c4ccccc34)ccc3c2oc2c4ccccc4c4ccccc4c32)cc1 ; c1ccc(-c2ccc(-c3ccc(N(c4ccccc4)c4ccc5c(oc6c7ccccc7c7ccccc7c56)c4-c4ccccc4)cc3)cc2)cc1 ; c1ccc(-c2c(N(c3ccccc3)c3ccc4c(c3)C3(c5ccccc5-c5ccccc53)c3ccccc3-4)ccc3c2oc2c4ccccc4c4ccccc4c32)cc1]

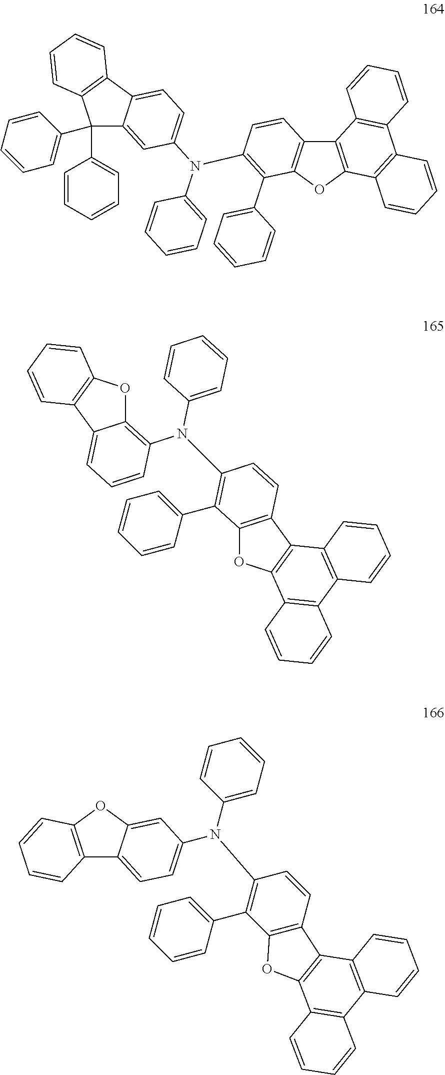 [SMILES: c1ccc(-c2c(N(c3ccccc3)c3ccc4c(c3)C(c3ccccc3)(c3ccccc3)c3ccccc3-4)ccc3c2oc2c4ccccc4c4ccccc4c32)cc1 ; c1ccc(-c2c(N(c3ccccc3)c3cccc4c3oc3ccccc34)ccc3c2oc2c4ccccc4c4ccccc4c32)cc1 ; c1ccc(-c2c(N(c3ccccc3)c3ccc4c(c3)oc3ccccc34)ccc3c2oc2c4ccccc4c4ccccc4c32)cc1]

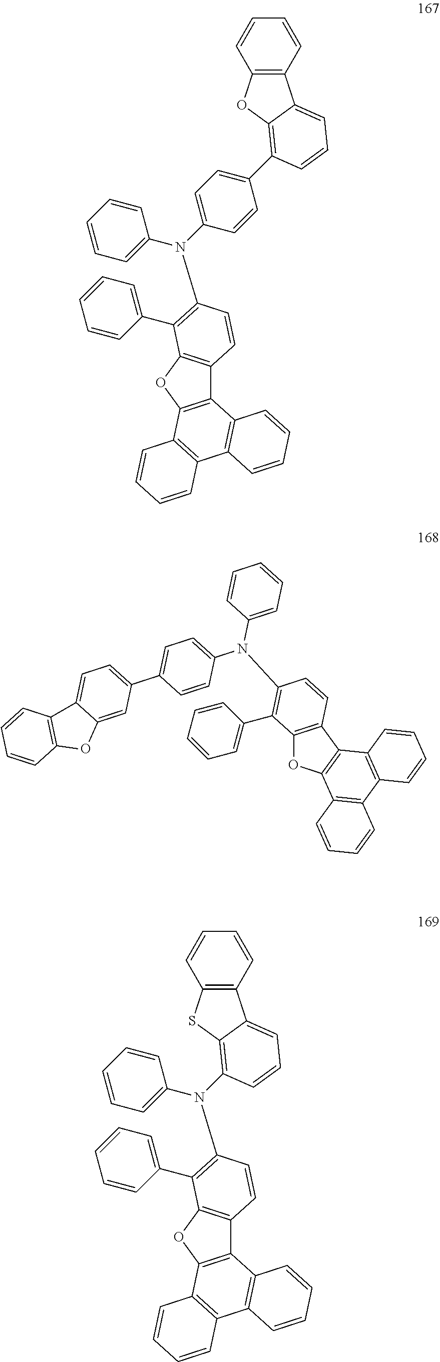 [SMILES: c1ccc(-c2c(N(c3ccccc3)c3ccc(-c4cccc5c4oc4ccccc45)cc3)ccc3c2oc2c4ccccc4c4ccccc4c32)cc1 ; c1ccc(-c2c(N(c3ccccc3)c3ccc(-c4ccc5c(c4)oc4ccccc45)cc3)ccc3c2oc2c4ccccc4c4ccccc4c32)cc1 ; c1ccc(-c2c(N(c3ccccc3)c3cccc4c3sc3ccccc34)ccc3c2oc2c4ccccc4c4ccccc4c32)cc1]

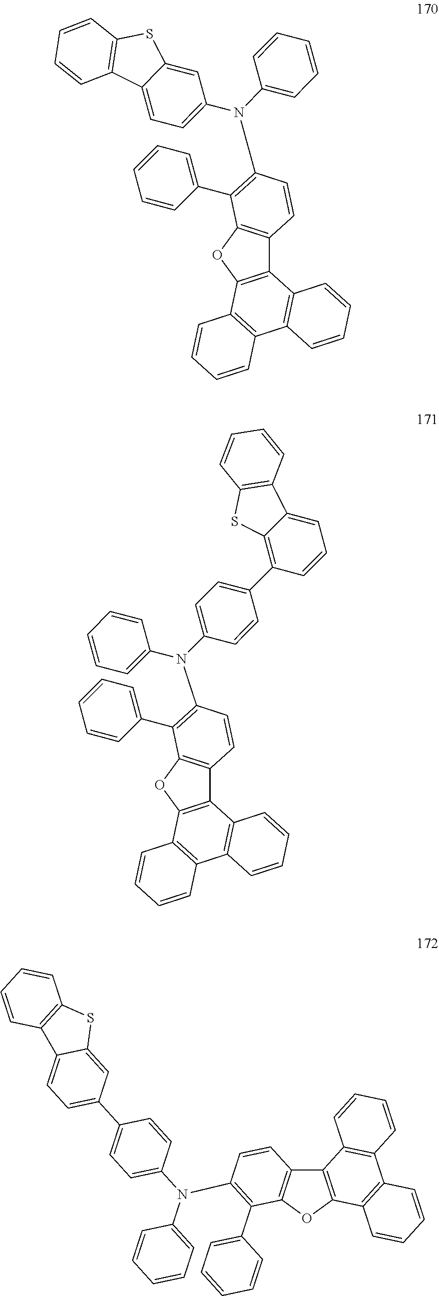 [SMILES: c1ccc(-c2c(N(c3ccccc3)c3ccc4c(c3)sc3ccccc34)ccc3c2oc2c4ccccc4c4ccccc4c32)cc1 ; c1ccc(-c2c(N(c3ccccc3)c3ccc(-c4cccc5c4sc4ccccc45)cc3)ccc3c2oc2c4ccccc4c4ccccc4c32)cc1 ; c1ccc(-c2c(N(c3ccccc3)c3ccc(-c4ccc5c(c4)sc4ccccc45)cc3)ccc3c2oc2c4ccccc4c4ccccc4c32)cc1]

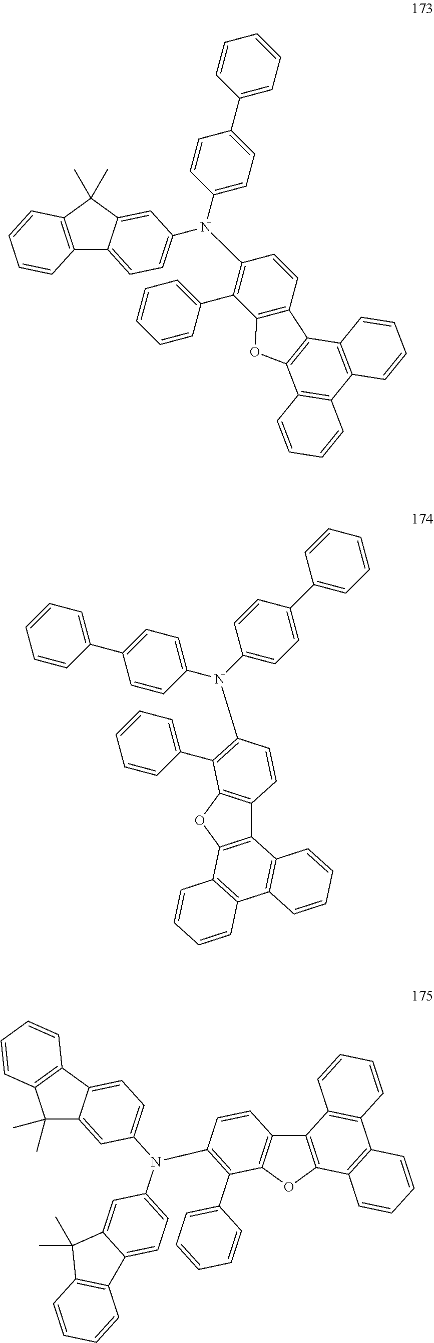 [SMILES: CC1(C)c2ccccc2-c2ccc(N(c3ccc(-c4ccccc4)cc3)c3ccc4c(oc5c6ccccc6c6ccccc6c45)c3-c3ccccc3)cc21 ; c1ccc(-c2ccc(N(c3ccc(-c4ccccc4)cc3)c3ccc4c(oc5c6ccccc6c6ccccc6c45)c3-c3ccccc3)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccccc3)cc21]

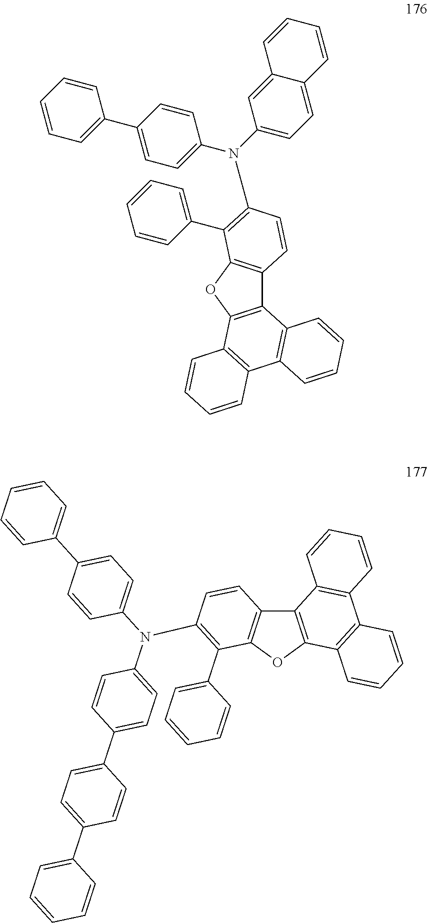 [SMILES: c1ccc(-c2ccc(N(c3ccc4ccccc4c3)c3ccc4c(oc5c6ccccc6c6ccccc6c45)c3-c3ccccc3)cc2)cc1 ; c1ccc(-c2ccc(-c3ccc(N(c4ccc(-c5ccccc5)cc4)c4ccc5c(oc6c7ccccc7c7ccccc7c56)c4-c4ccccc4)cc3)cc2)cc1]

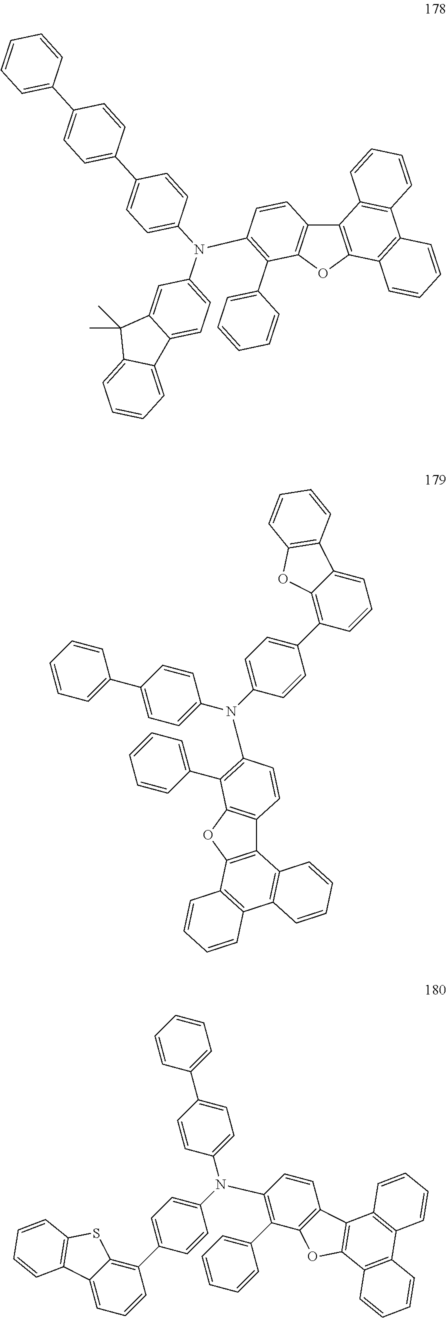 [SMILES: CC1(C)c2ccccc2-c2ccc(N(c3ccc(-c4ccc(-c5ccccc5)cc4)cc3)c3ccc4c(oc5c6ccccc6c6ccccc6c45)c3-c3ccccc3)cc21 ; c1ccc(-c2ccc(N(c3ccc(-c4cccc5c4oc4ccccc45)cc3)c3ccc4c(oc5c6ccccc6c6ccccc6c45)c3-c3ccccc3)cc2)cc1 ; c1ccc(-c2ccc(N(c3ccc(-c4cccc5c4sc4ccccc45)cc3)c3ccc4c(oc5c6ccccc6c6ccccc6c45)c3-c3ccccc3)cc2)cc1]

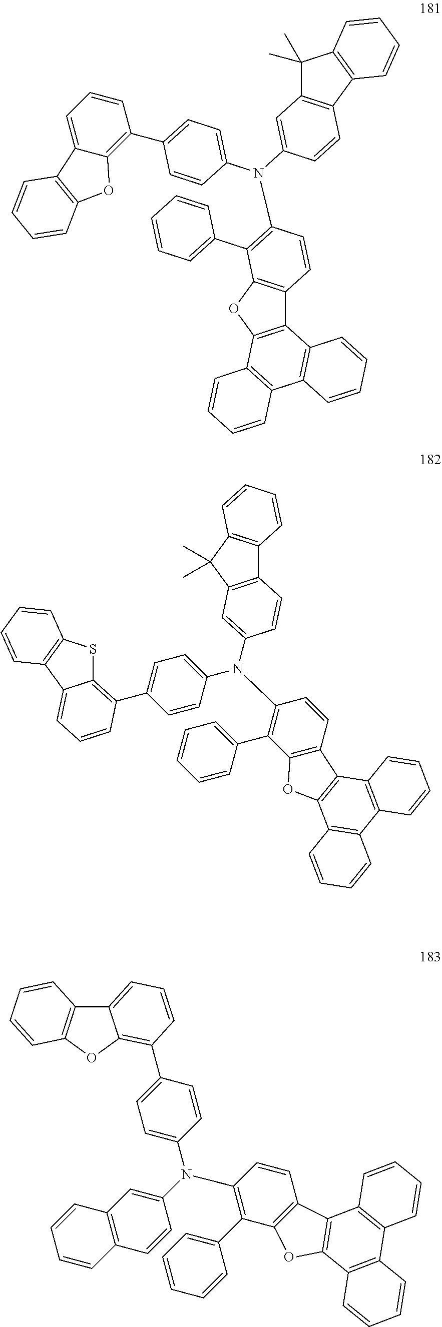 [SMILES: CC1(C)c2ccccc2-c2ccc(N(c3ccc(-c4cccc5c4oc4ccccc45)cc3)c3ccc4c(oc5c6ccccc6c6ccccc6c45)c3-c3ccccc3)cc21 ; CC1(C)c2ccccc2-c2ccc(N(c3ccc(-c4cccc5c4sc4ccccc45)cc3)c3ccc4c(oc5c6ccccc6c6ccccc6c45)c3-c3ccccc3)cc21 ; c1ccc(-c2c(N(c3ccc(-c4cccc5c4oc4ccccc45)cc3)c3ccc4ccccc4c3)ccc3c2oc2c4ccccc4c4ccccc4c32)cc1]

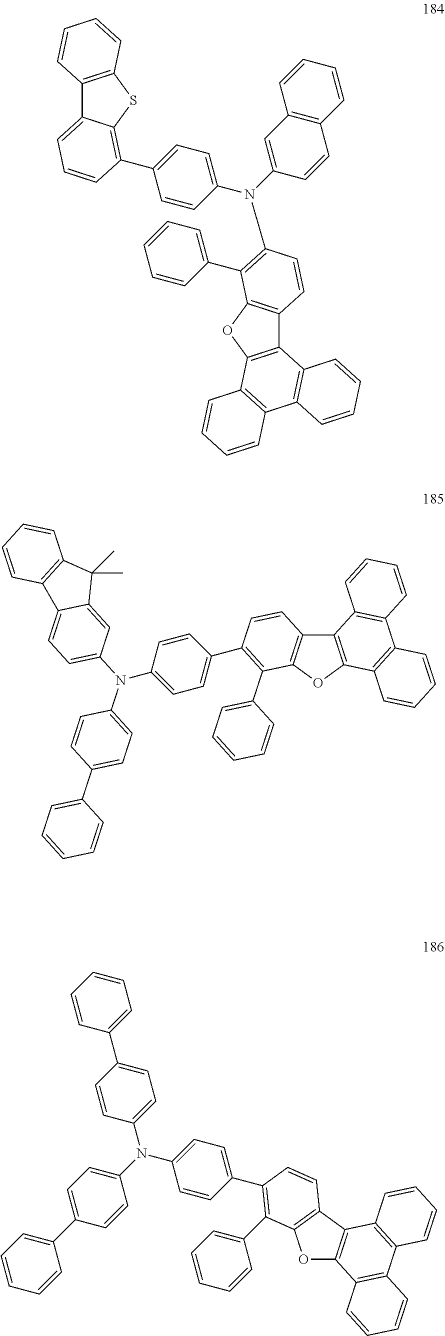 [SMILES: c1ccc(-c2c(N(c3ccc(-c4cccc5c4sc4ccccc45)cc3)c3ccc4ccccc4c3)ccc3c2oc2c4ccccc4c4ccccc4c32)cc1 ; CC1(C)c2ccccc2-c2ccc(N(c3ccc(-c4ccccc4)cc3)c3ccc(-c4ccc5c(oc6c7ccccc7c7ccccc7c56)c4-c4ccccc4)cc3)cc21 ; c1ccc(-c2ccc(N(c3ccc(-c4ccccc4)cc3)c3ccc(-c4ccc5c(oc6c7ccccc7c7ccccc7c56)c4-c4ccccc4)cc3)cc2)cc1]

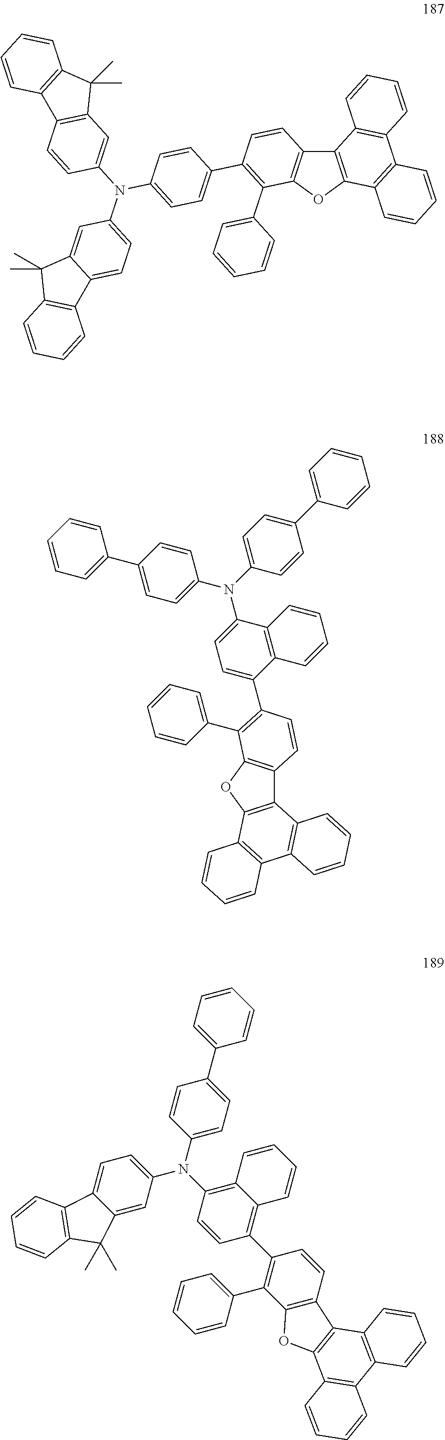 [SMILES: CC1(C)c2ccccc2-c2ccc(N(c3ccc(-c4ccc5c(oc6c7ccccc7c7ccccc7c56)c4-c4ccccc4)cc3)c3ccc4c(c3)C(C)(C)c3ccccc3-4)cc21 ; c1ccc(-c2ccc(N(c3ccc(-c4ccccc4)cc3)c3ccc(-c4ccc5c(oc6c7ccccc7c7ccccc7c56)c4-c4ccccc4)c4ccccc34)cc2)cc1 ; CC1(C)c2ccccc2-c2ccc(N(c3ccc(-c4ccccc4)cc3)c3ccc(-c4ccc5c(oc6c7ccccc7c7ccccc7c56)c4-c4ccccc4)c4ccccc34)cc21]

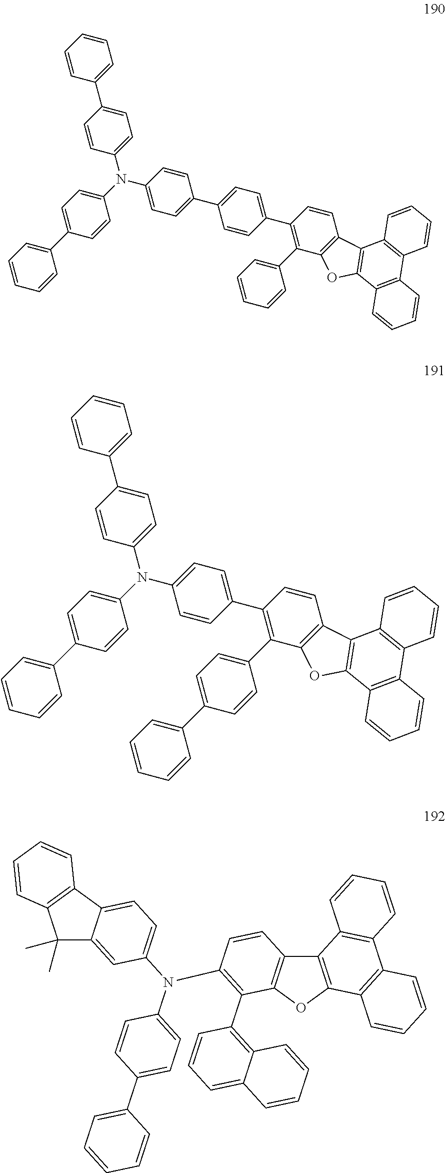 [SMILES: c1ccc(-c2ccc(N(c3ccc(-c4ccccc4)cc3)c3ccc(-c4ccc(-c5ccc6c(oc7c8ccccc8c8ccccc8c67)c5-c5ccccc5)cc4)cc3)cc2)cc1 ; c1ccc(-c2ccc(-c3c(-c4ccc(N(c5ccc(-c6ccccc6)cc5)c5ccc(-c6ccccc6)cc5)cc4)ccc4c3oc3c5ccccc5c5ccccc5c43)cc2)cc1 ; CC1(C)c2ccccc2-c2ccc(N(c3ccc(-c4ccccc4)cc3)c3ccc4c(oc5c6ccccc6c6ccccc6c45)c3-c3cccc4ccccc34)cc21]

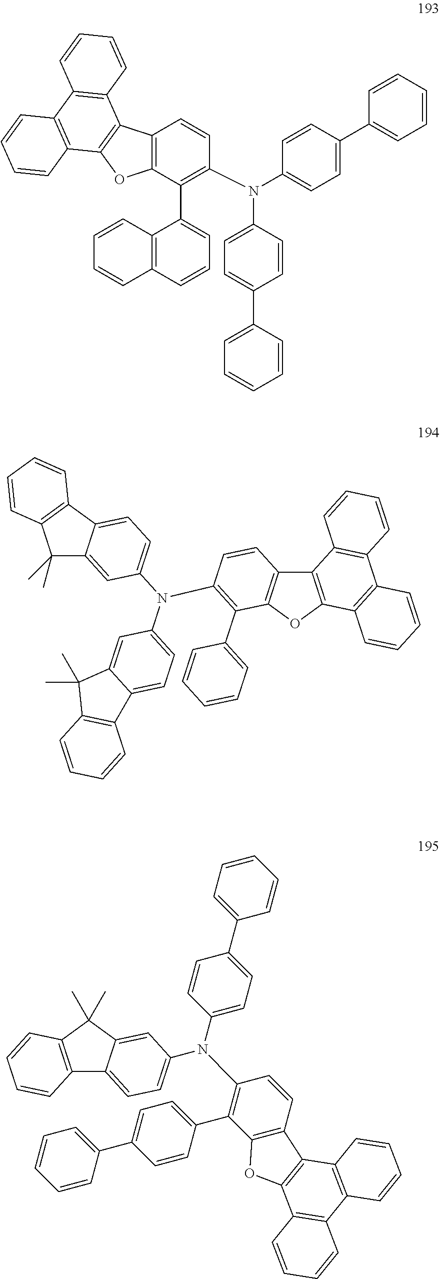 [SMILES: c1ccc(-c2ccc(N(c3ccc(-c4ccccc4)cc3)c3ccc4c(oc5c6ccccc6c6ccccc6c45)c3-c3cccc4ccccc34)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccccc3)cc21 ; CC1(C)c2ccccc2-c2ccc(N(c3ccc(-c4ccccc4)cc3)c3ccc4c(oc5c6ccccc6c6ccccc6c45)c3-c3ccc(-c4ccccc4)cc3)cc21]

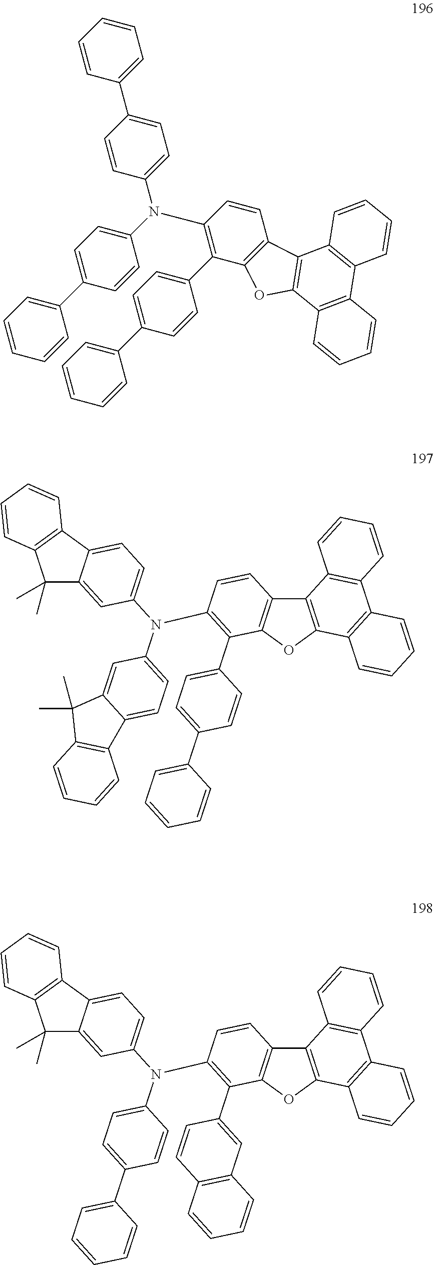 [SMILES: c1ccc(-c2ccc(-c3c(N(c4ccc(-c5ccccc5)cc4)c4ccc(-c5ccccc5)cc4)ccc4c3oc3c5ccccc5c5ccccc5c43)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccc(-c4ccccc4)cc3)cc21 ; CC1(C)c2ccccc2-c2ccc(N(c3ccc(-c4ccccc4)cc3)c3ccc4c(oc5c6ccccc6c6ccccc6c45)c3-c3ccc4ccccc4c3)cc21]

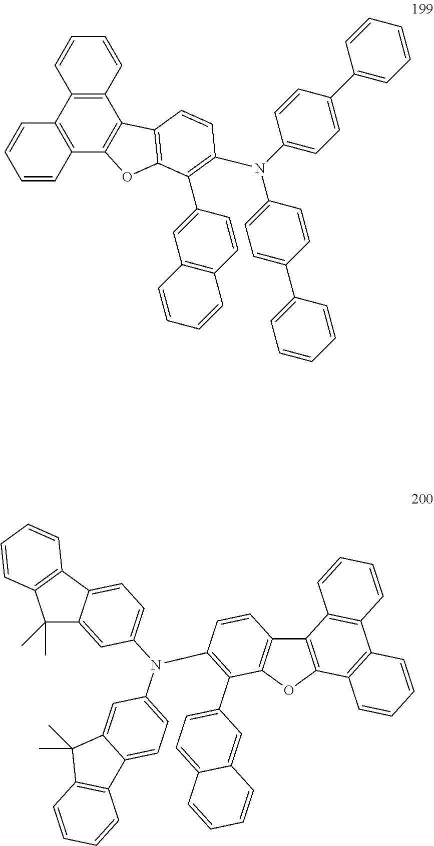 [SMILES: c1ccc(-c2ccc(N(c3ccc(-c4ccccc4)cc3)c3ccc4c(oc5c6ccccc6c6ccccc6c45)c3-c3ccc4ccccc4c3)cc2)cc1 ; CC1(C)c2ccccc2-c2ccc(N(c3ccc4c(c3)C(C)(C)c3ccccc3-4)c3ccc4c(oc5c6ccccc6c6ccccc6c45)c3-c3ccc4ccccc4c3)cc21]

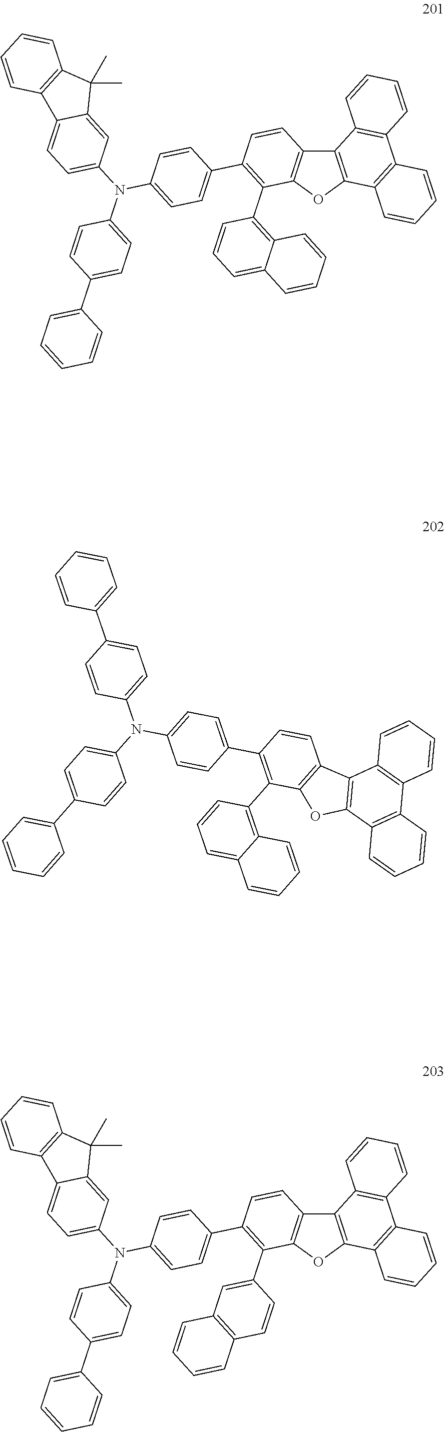 [SMILES: CC1(C)c2ccccc2-c2ccc(N(c3ccc(-c4ccccc4)cc3)c3ccc(-c4ccc5c(oc6c7ccccc7c7ccccc7c56)c4-c4cccc5ccccc45)cc3)cc21 ; c1ccc(-c2ccc(N(c3ccc(-c4ccccc4)cc3)c3ccc(-c4ccc5c(oc6c7ccccc7c7ccccc7c56)c4-c4cccc5ccccc45)cc3)cc2)cc1 ; CC1(C)c2ccccc2-c2ccc(N(c3ccc(-c4ccccc4)cc3)c3ccc(-c4ccc5c(oc6c7ccccc7c7ccccc7c56)c4-c4ccc5ccccc5c4)cc3)cc21]

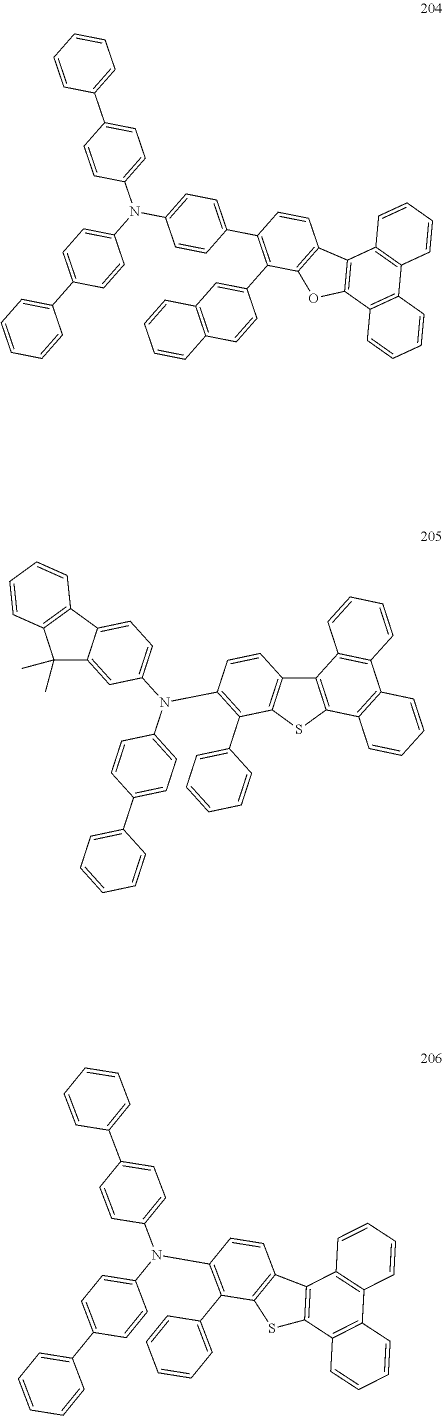 [SMILES: c1ccc(-c2ccc(N(c3ccc(-c4ccccc4)cc3)c3ccc(-c4ccc5c(oc6c7ccccc7c7ccccc7c56)c4-c4ccc5ccccc5c4)cc3)cc2)cc1 ; CC1(C)c2ccccc2-c2ccc(N(c3ccc(-c4ccccc4)cc3)c3ccc4c(sc5c6ccccc6c6ccccc6c45)c3-c3ccccc3)cc21 ; c1ccc(-c2ccc(N(c3ccc(-c4ccccc4)cc3)c3ccc4c(sc5c6ccccc6c6ccccc6c45)c3-c3ccccc3)cc2)cc1]

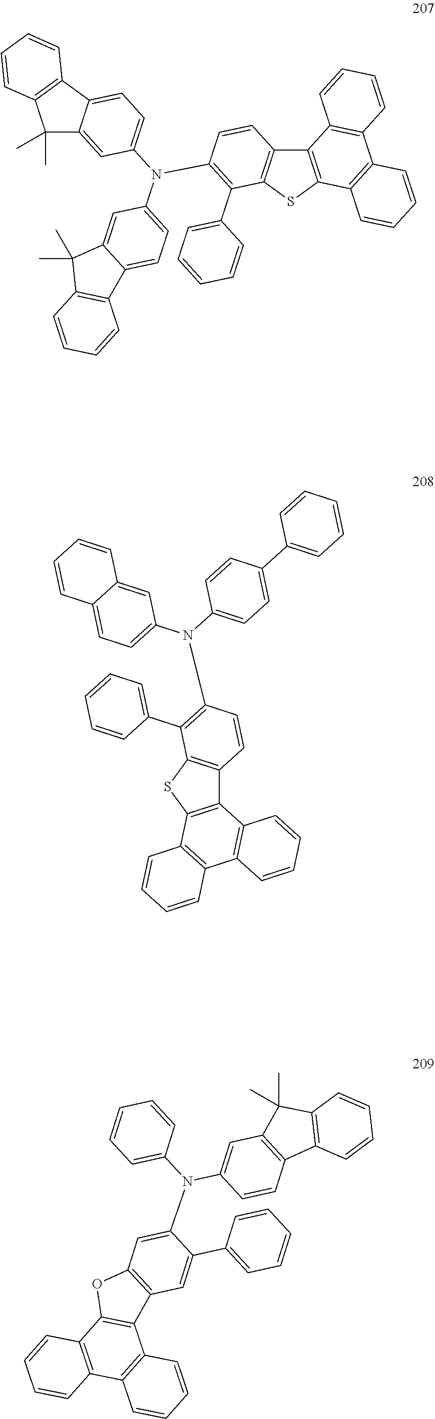 [SMILES: CC1(C)c2ccccc2-c2ccc(N(c3ccc4c(c3)C(C)(C)c3ccccc3-4)c3ccc4c(sc5c6ccccc6c6ccccc6c45)c3-c3ccccc3)cc21 ; c1ccc(-c2ccc(N(c3ccc4ccccc4c3)c3ccc4c(sc5c6ccccc6c6ccccc6c45)c3-c3ccccc3)cc2)cc1 ; CC1(C)c2ccccc2-c2ccc(N(c3ccccc3)c3cc4oc5c6ccccc6c6ccccc6c5c4cc3-c3ccccc3)cc21]

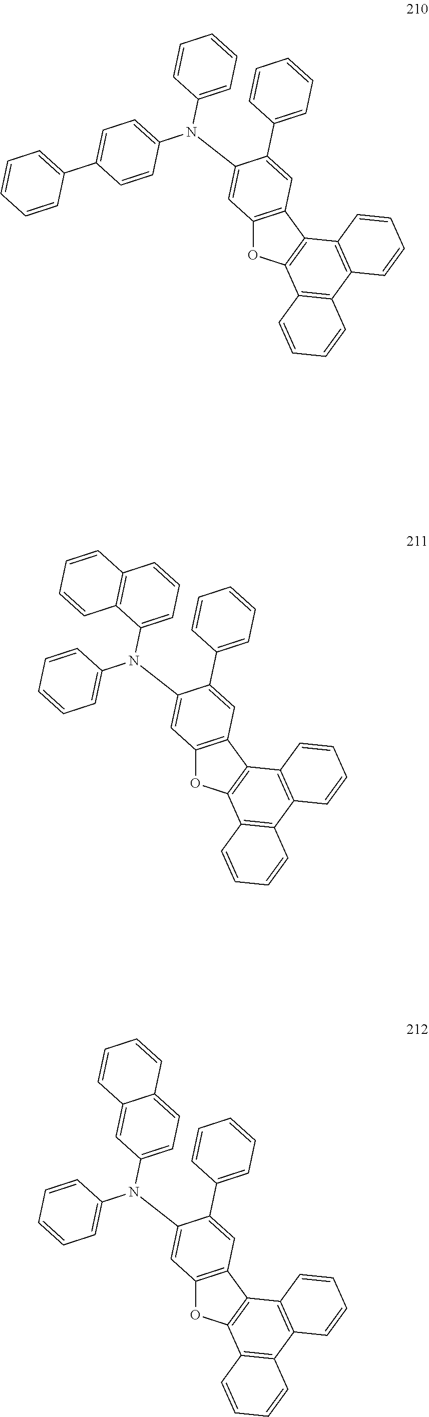 [SMILES: c1ccc(-c2ccc(N(c3ccccc3)c3cc4oc5c6ccccc6c6ccccc6c5c4cc3-c3ccccc3)cc2)cc1 ; c1ccc(-c2cc3c(cc2N(c2ccccc2)c2cccc4ccccc24)oc2c4ccccc4c4ccccc4c32)cc1 ; c1ccc(-c2cc3c(cc2N(c2ccccc2)c2ccc4ccccc4c2)oc2c4ccccc4c4ccccc4c32)cc1]

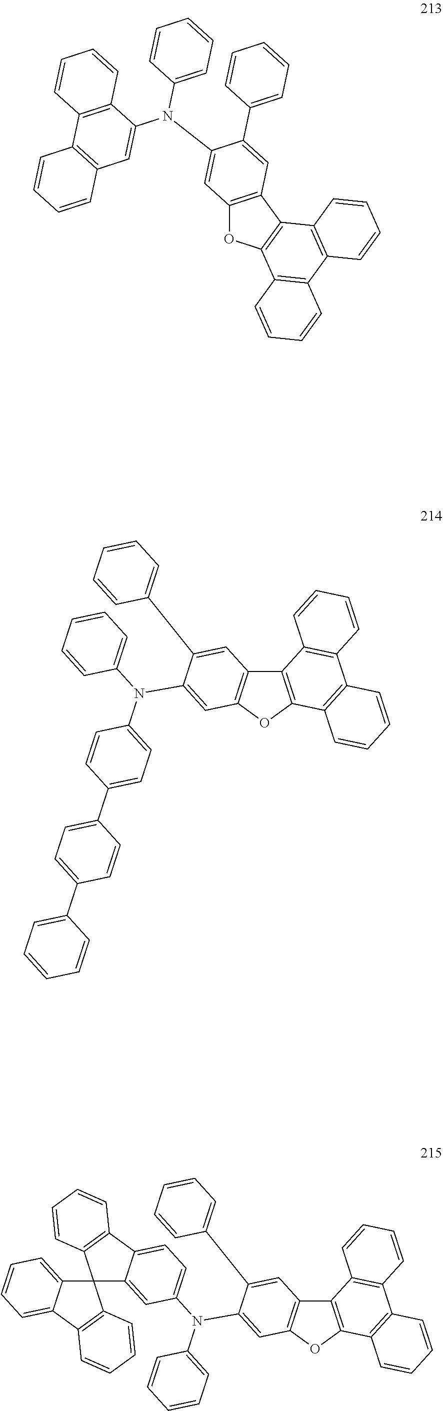 [SMILES: c1ccc(-c2cc3c(cc2N(c2ccccc2)c2cc4ccccc4c4ccccc24)oc2c4ccccc4c4ccccc4c32)cc1 ; c1ccc(-c2ccc(-c3ccc(N(c4ccccc4)c4cc5oc6c7ccccc7c7ccccc7c6c5cc4-c4ccccc4)cc3)cc2)cc1 ; c1ccc(-c2cc3c(cc2N(c2ccccc2)c2ccc4c(c2)C2(c5ccccc5-c5ccccc52)c2ccccc2-4)oc2c4ccccc4c4ccccc4c32)cc1]

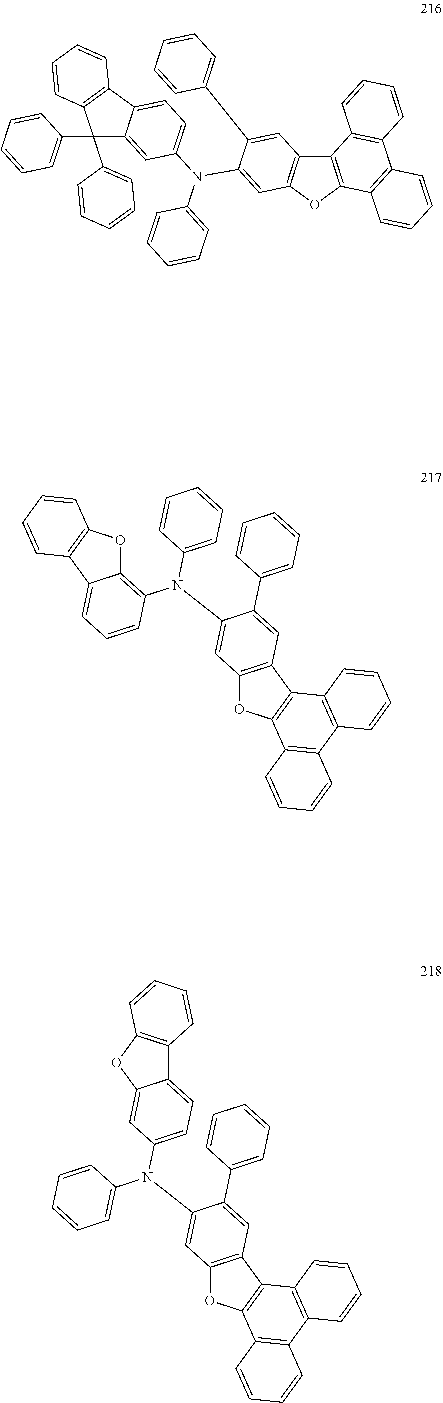 [SMILES: c1ccc(-c2cc3c(cc2N(c2ccccc2)c2ccc4c(c2)C(c2ccccc2)(c2ccccc2)c2ccccc2-4)oc2c4ccccc4c4ccccc4c32)cc1 ; c1ccc(-c2cc3c(cc2N(c2ccccc2)c2cccc4c2oc2ccccc24)oc2c4ccccc4c4ccccc4c32)cc1 ; c1ccc(-c2cc3c(cc2N(c2ccccc2)c2ccc4c(c2)oc2ccccc24)oc2c4ccccc4c4ccccc4c32)cc1]

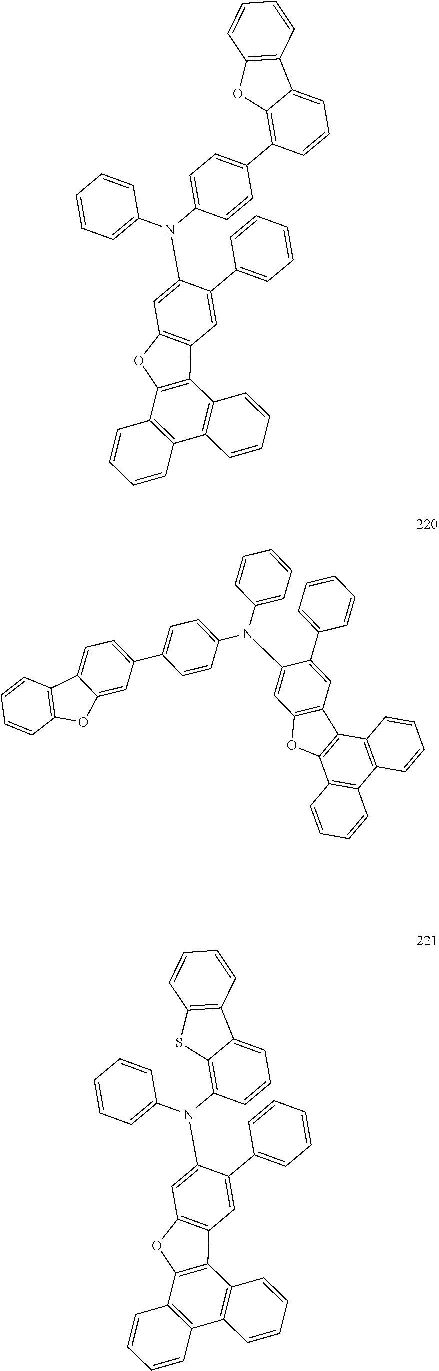 [SMILES: c1ccc(-c2cc3c(cc2N(c2ccccc2)c2ccc(-c4cccc5c4oc4ccccc45)cc2)oc2c4ccccc4c4ccccc4c32)cc1 ; c1ccc(-c2cc3c(cc2N(c2ccccc2)c2ccc(-c4ccc5c(c4)oc4ccccc45)cc2)oc2c4ccccc4c4ccccc4c32)cc1 ; c1ccc(-c2cc3c(cc2N(c2ccccc2)c2cccc4c2sc2ccccc24)oc2c4ccccc4c4ccccc4c32)cc1]

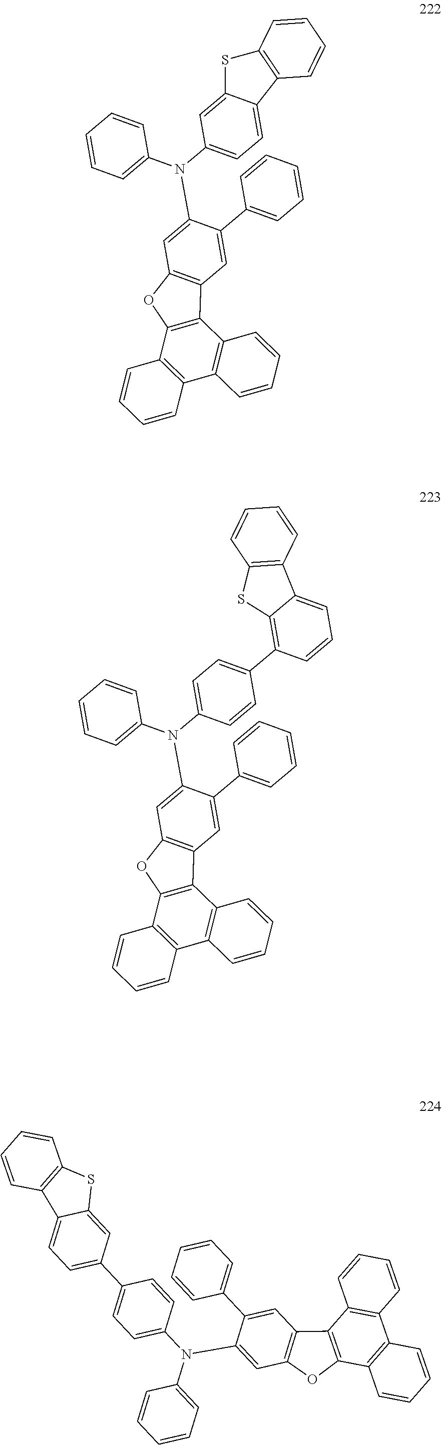 [SMILES: c1ccc(-c2cc3c(cc2N(c2ccccc2)c2ccc4c(c2)sc2ccccc24)oc2c4ccccc4c4ccccc4c32)cc1 ; c1ccc(-c2cc3c(cc2N(c2ccccc2)c2ccc(-c4cccc5c4sc4ccccc45)cc2)oc2c4ccccc4c4ccccc4c32)cc1 ; c1ccc(-c2cc3c(cc2N(c2ccccc2)c2ccc(-c4ccc5c(c4)sc4ccccc45)cc2)oc2c4ccccc4c4ccccc4c32)cc1]

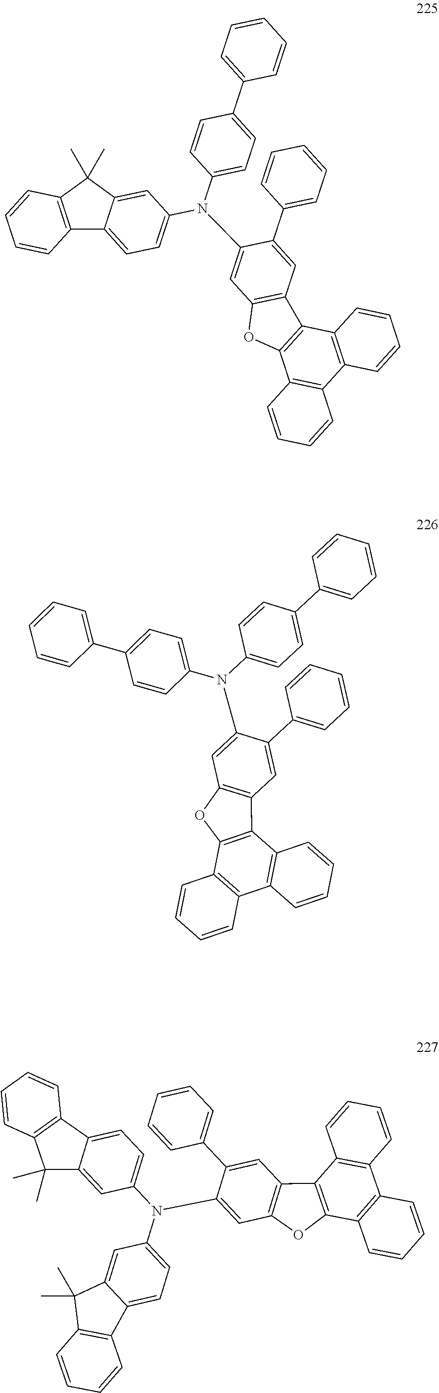 [SMILES: CC1(C)c2ccccc2-c2ccc(N(c3ccc(-c4ccccc4)cc3)c3cc4oc5c6ccccc6c6ccccc6c5c4cc3-c3ccccc3)cc21 ; c1ccc(-c2ccc(N(c3ccc(-c4ccccc4)cc3)c3cc4oc5c6ccccc6c6ccccc6c5c4cc3-c3ccccc3)cc2)cc1 ; CC1(C)c2ccccc2-c2ccc(N(c3ccc4c(c3)C(C)(C)c3ccccc3-4)c3cc4oc5c6ccccc6c6ccccc6c5c4cc3-c3ccccc3)cc21]

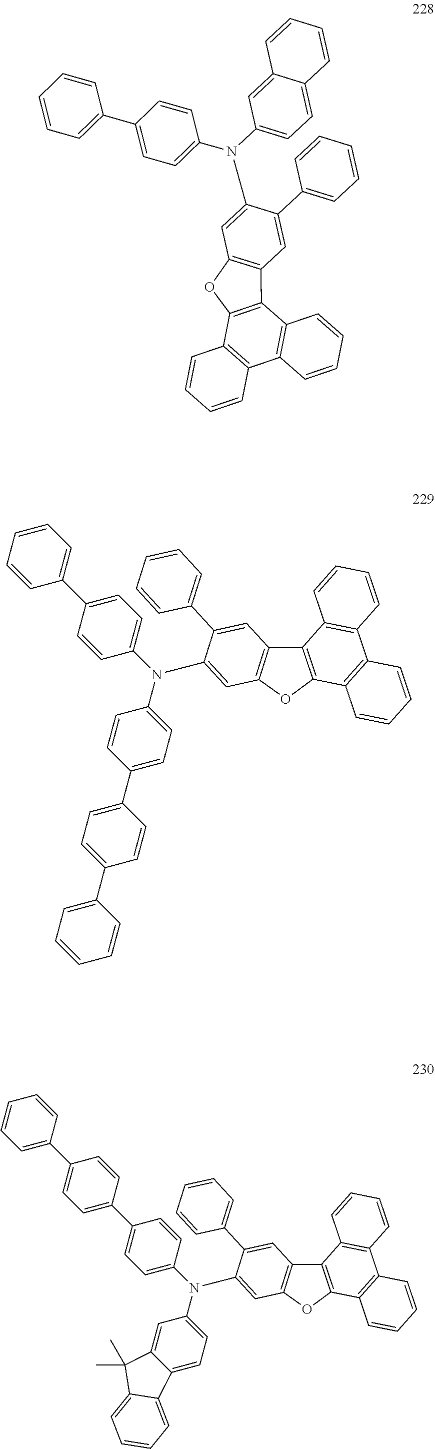

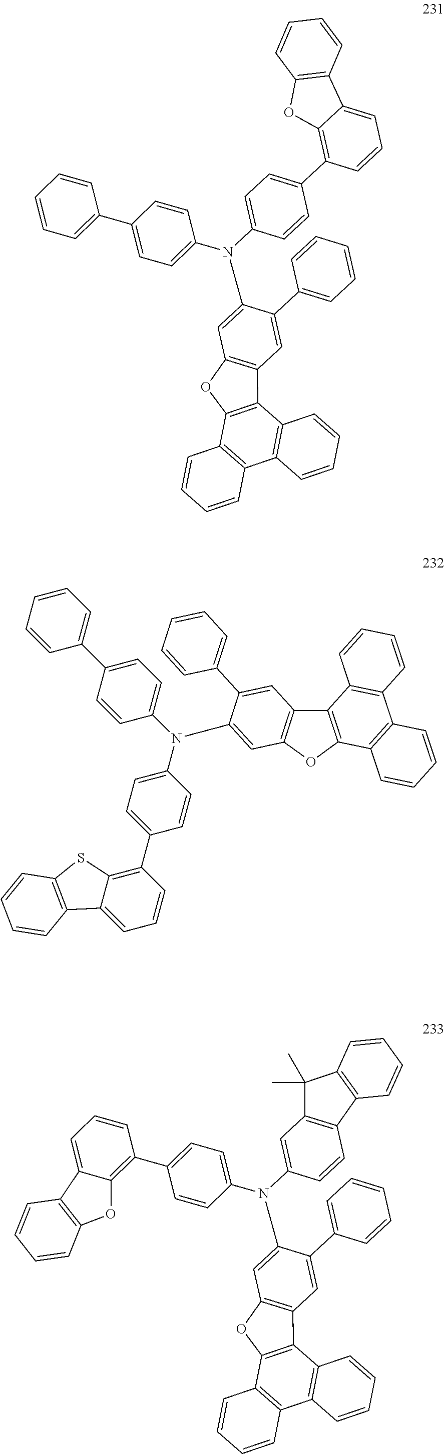 [SMILES: c1ccc(-c2ccc(N(c3ccc(-c4cccc5c4oc4ccccc45)cc3)c3cc4oc5c6ccccc6c6ccccc6c5c4cc3-c3ccccc3)cc2)cc1 ; c1ccc(-c2ccc(N(c3ccc(-c4cccc5c4sc4ccccc45)cc3)c3cc4oc5c6ccccc6c6ccccc6c5c4cc3-c3ccccc3)cc2)cc1 ; CC1(C)c2ccccc2-c2ccc(N(c3ccc(-c4cccc5c4oc4ccccc45)cc3)c3cc4oc5c6ccccc6c6ccccc6c5c4cc3-c3ccccc3)cc21]

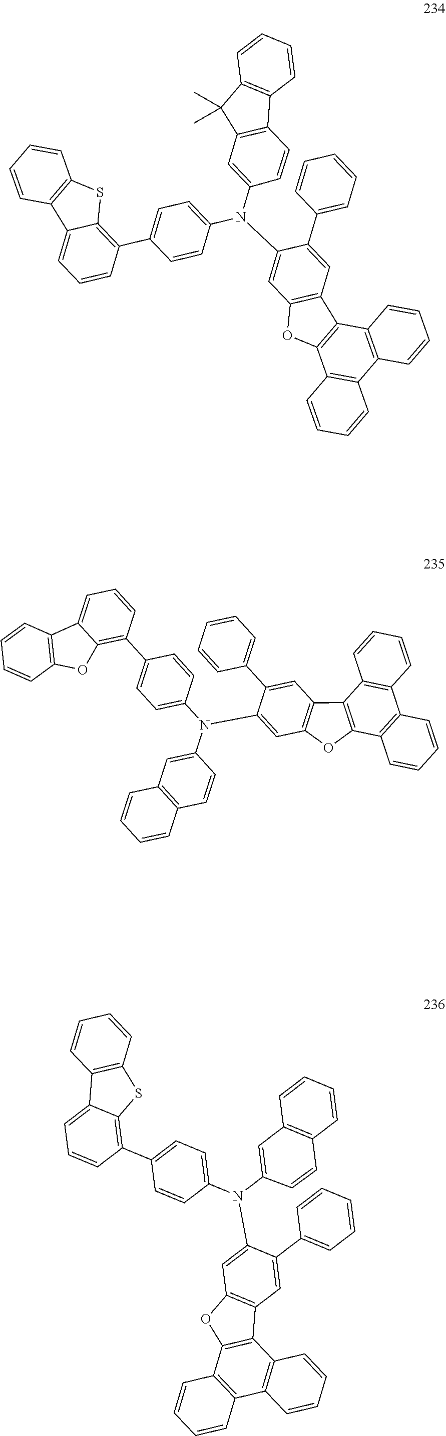 [SMILES: CC1(C)c2ccccc2-c2ccc(N(c3ccc(-c4cccc5c4sc4ccccc45)cc3)c3cc4oc5c6ccccc6c6ccccc6c5c4cc3-c3ccccc3)cc21 ; c1ccc(-c2cc3c(cc2N(c2ccc(-c4cccc5c4oc4ccccc45)cc2)c2ccc4ccccc4c2)oc2c4ccccc4c4ccccc4c32)cc1 ; c1ccc(-c2cc3c(cc2N(c2ccc(-c4cccc5c4sc4ccccc45)cc2)c2ccc4ccccc4c2)oc2c4ccccc4c4ccccc4c32)cc1]

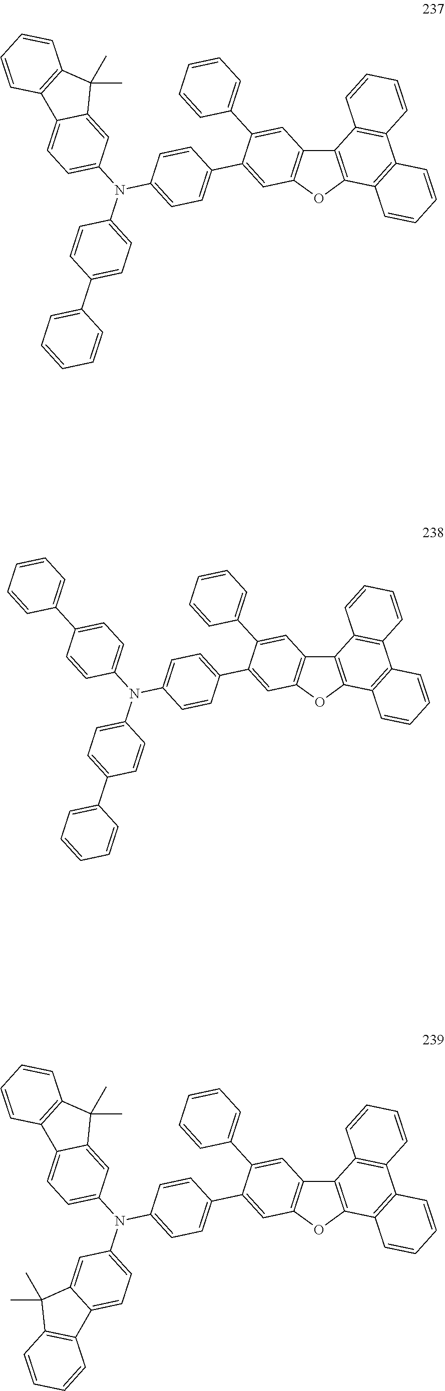 [SMILES: CC1(C)c2ccccc2-c2ccc(N(c3ccc(-c4ccccc4)cc3)c3ccc(-c4cc5oc6c7ccccc7c7ccccc7c6c5cc4-c4ccccc4)cc3)cc21 ; c1ccc(-c2ccc(N(c3ccc(-c4ccccc4)cc3)c3ccc(-c4cc5oc6c7ccccc7c7ccccc7c6c5cc4-c4ccccc4)cc3)cc2)cc1 ; CC1(C)c2ccccc2-c2ccc(N(c3ccc(-c4cc5oc6c7ccccc7c7ccccc7c6c5cc4-c4ccccc4)cc3)c3ccc4c(c3)C(C)(C)c3ccccc3-4)cc21]

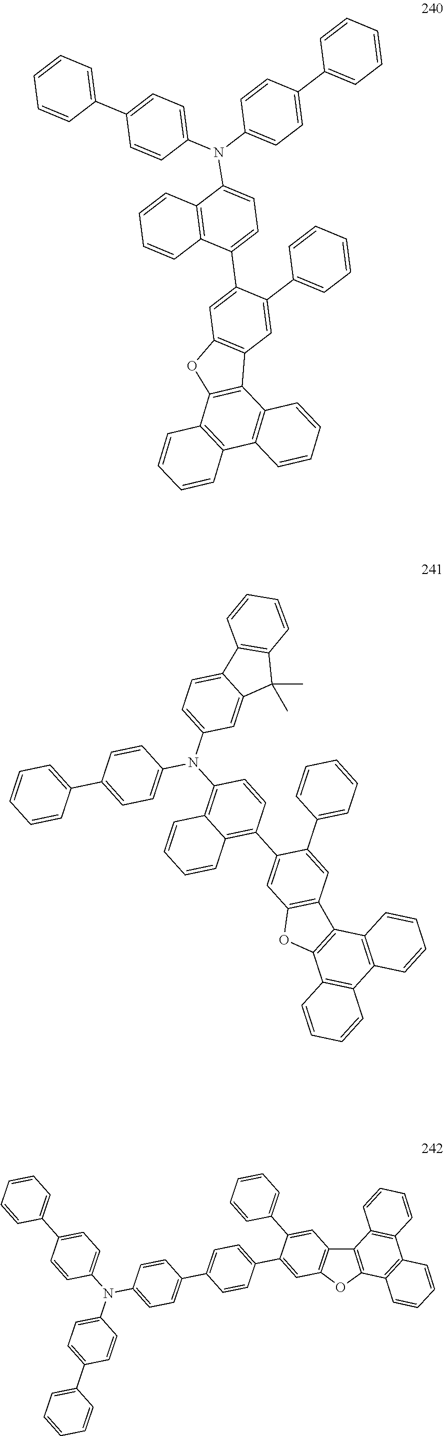

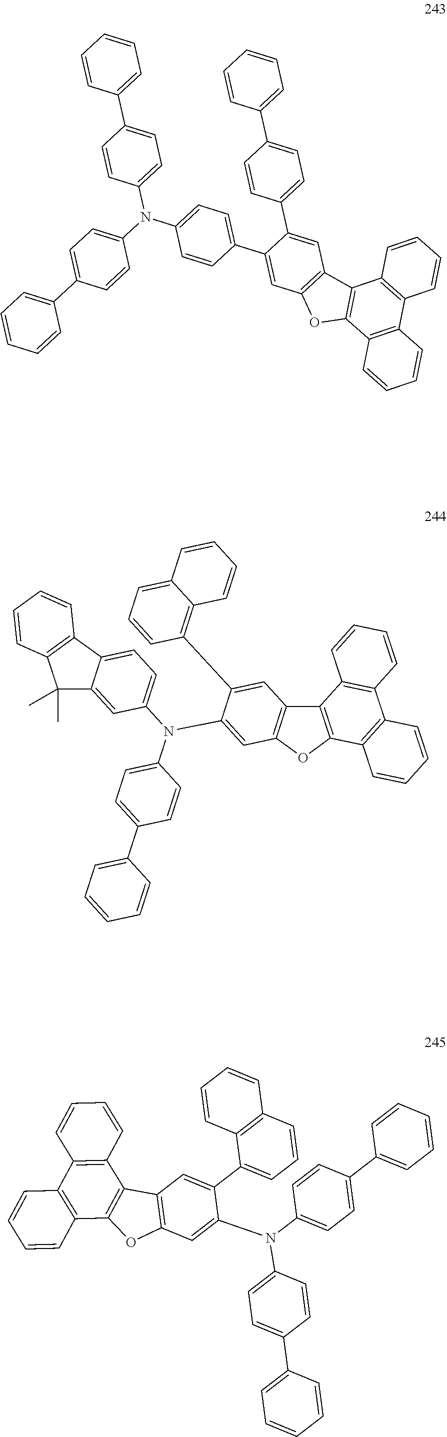

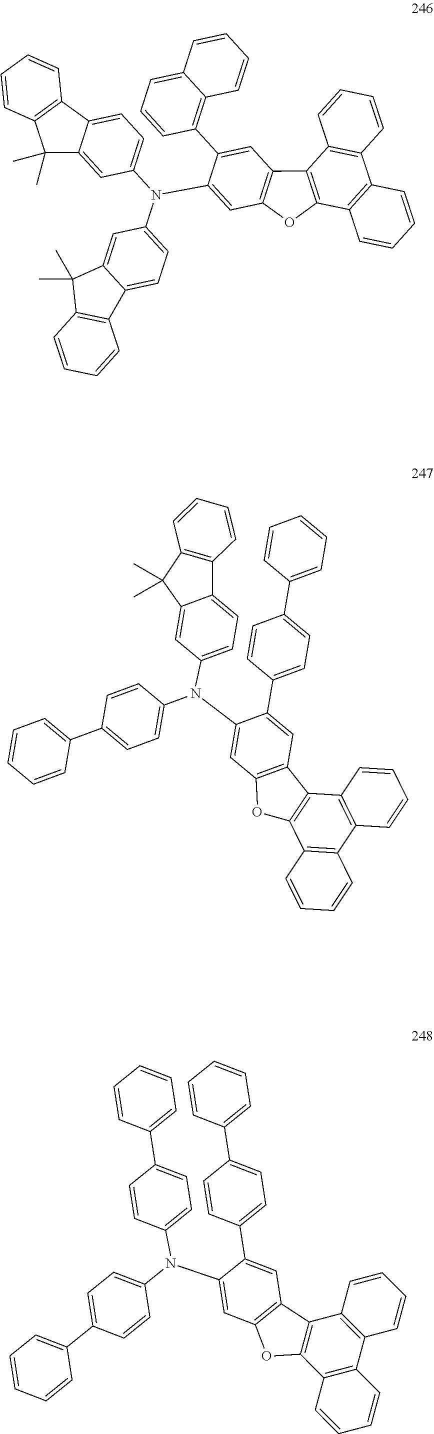 [SMILES: CC1(C)c2ccccc2-c2ccc(N(c3ccc4c(c3)C(C)(C)c3ccccc3-4)c3cc4oc5c6ccccc6c6ccccc6c5c4cc3-c3cccc4ccccc34)cc21 ; CC1(C)c2ccccc2-c2ccc(N(c3ccc(-c4ccccc4)cc3)c3cc4oc5c6ccccc6c6ccccc6c5c4cc3-c3ccc(-c4ccccc4)cc3)cc21 ; c1ccc(-c2ccc(-c3cc4c(cc3N(c3ccc(-c5ccccc5)cc3)c3ccc(-c5ccccc5)cc3)oc3c5ccccc5c5ccccc5c43)cc2)cc1]

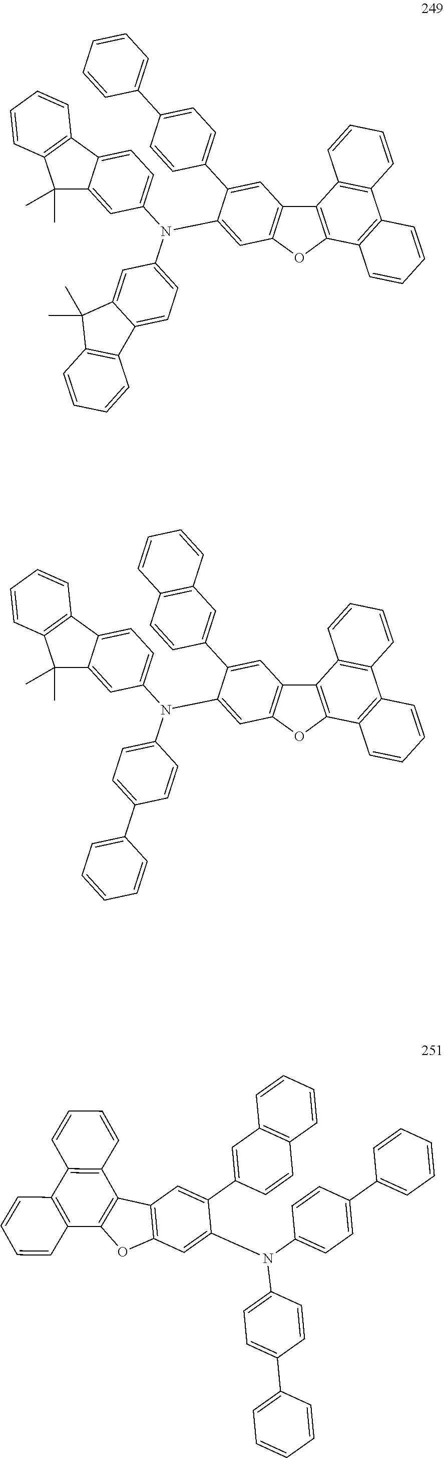 [SMILES: CC1(C)c2ccccc2-c2ccc(N(c3ccc4c(c3)C(C)(C)c3ccccc3-4)c3cc4oc5c6ccccc6c6ccccc6c5c4cc3-c3ccc(-c4ccccc4)cc3)cc21 ; CC1(C)c2ccccc2-c2ccc(N(c3ccc(-c4ccccc4)cc3)c3cc4oc5c6ccccc6c6ccccc6c5c4cc3-c3ccc4ccccc4c3)cc21 ; c1ccc(-c2ccc(N(c3ccc(-c4ccccc4)cc3)c3cc4oc5c6ccccc6c6ccccc6c5c4cc3-c3ccc4ccccc4c3)cc2)cc1]

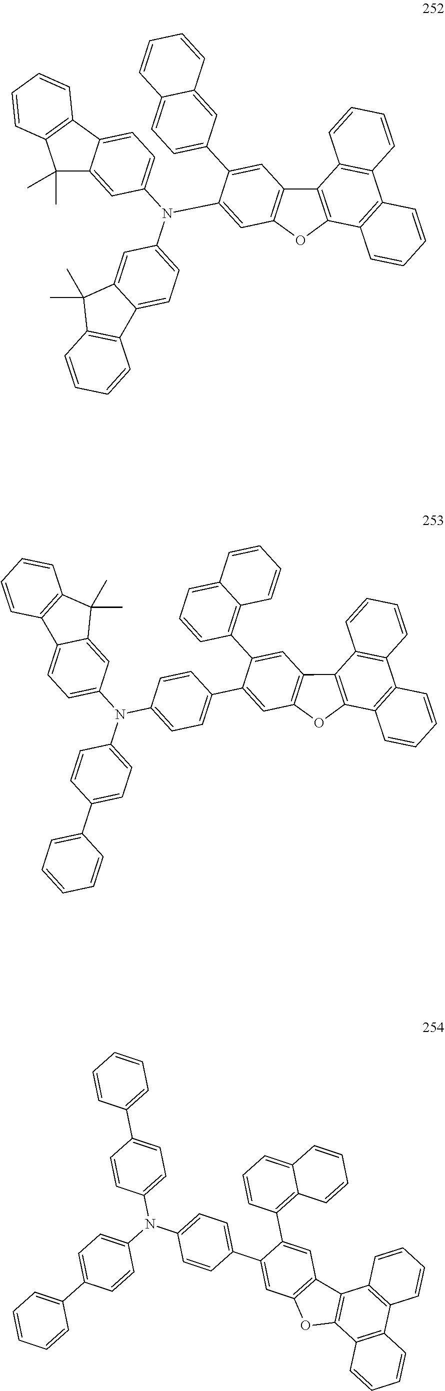 [SMILES: CC1(C)c2ccccc2-c2ccc(N(c3ccc4c(c3)C(C)(C)c3ccccc3-4)c3cc4oc5c6ccccc6c6ccccc6c5c4cc3-c3ccc4ccccc4c3)cc21 ; CC1(C)c2ccccc2-c2ccc(N(c3ccc(-c4ccccc4)cc3)c3ccc(-c4cc5oc6c7ccccc7c7ccccc7c6c5cc4-c4cccc5ccccc45)cc3)cc21 ; c1ccc(-c2ccc(N(c3ccc(-c4ccccc4)cc3)c3ccc(-c4cc5oc6c7ccccc7c7ccccc7c6c5cc4-c4cccc5ccccc45)cc3)cc2)cc1]

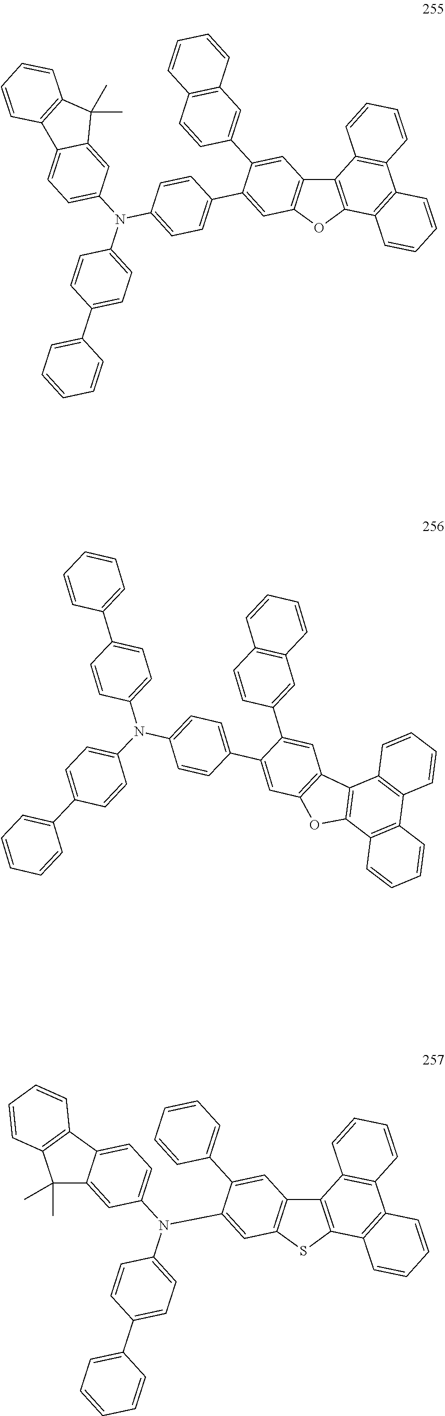 [SMILES: CC1(C)c2ccccc2-c2ccc(N(c3ccc(-c4ccccc4)cc3)c3ccc(-c4cc5oc6c7ccccc7c7ccccc7c6c5cc4-c4ccc5ccccc5c4)cc3)cc21 ; c1ccc(-c2ccc(N(c3ccc(-c4ccccc4)cc3)c3ccc(-c4cc5oc6c7ccccc7c7ccccc7c6c5cc4-c4ccc5ccccc5c4)cc3)cc2)cc1 ; CC1(C)c2ccccc2-c2ccc(N(c3ccc(-c4ccccc4)cc3)c3cc4sc5c6ccccc6c6ccccc6c5c4cc3-c3ccccc3)cc21]

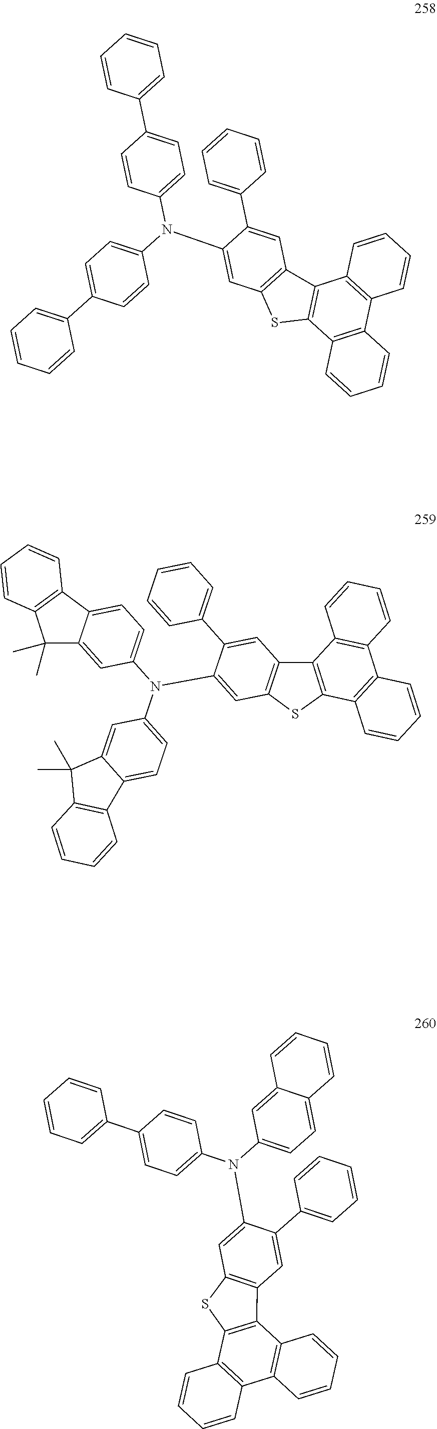 [SMILES: c1ccc(-c2ccc(N(c3ccc(-c4ccccc4)cc3)c3cc4sc5c6ccccc6c6ccccc6c5c4cc3-c3ccccc3)cc2)cc1 ; CC1(C)c2ccccc2-c2ccc(N(c3ccc4c(c3)C(C)(C)c3ccccc3-4)c3cc4sc5c6ccccc6c6ccccc6c5c4cc3-c3ccccc3)cc21 ; c1ccc(-c2ccc(N(c3ccc4ccccc4c3)c3cc4sc5c6ccccc6c6ccccc6c5c4cc3-c3ccccc3)cc2)cc1]

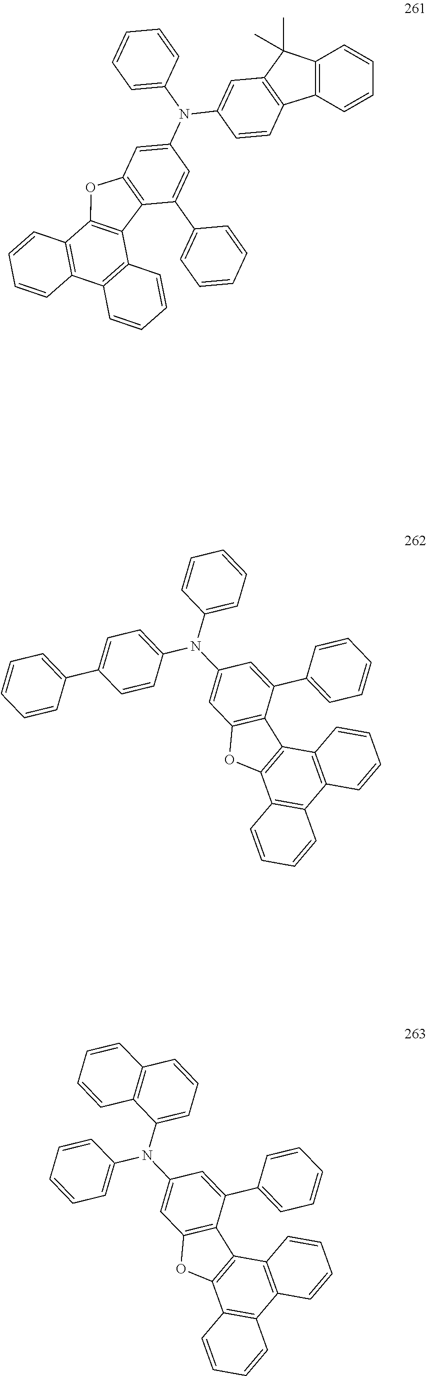 [SMILES: CC1(C)c2ccccc2-c2ccc(N(c3ccccc3)c3cc(-c4ccccc4)c4c(c3)oc3c5ccccc5c5ccccc5c34)cc21 ; c1ccc(-c2ccc(N(c3ccccc3)c3cc(-c4ccccc4)c4c(c3)oc3c5ccccc5c5ccccc5c34)cc2)cc1 ; c1ccc(-c2cc(N(c3ccccc3)c3cccc4ccccc34)cc3oc4c5ccccc5c5ccccc5c4c23)cc1]

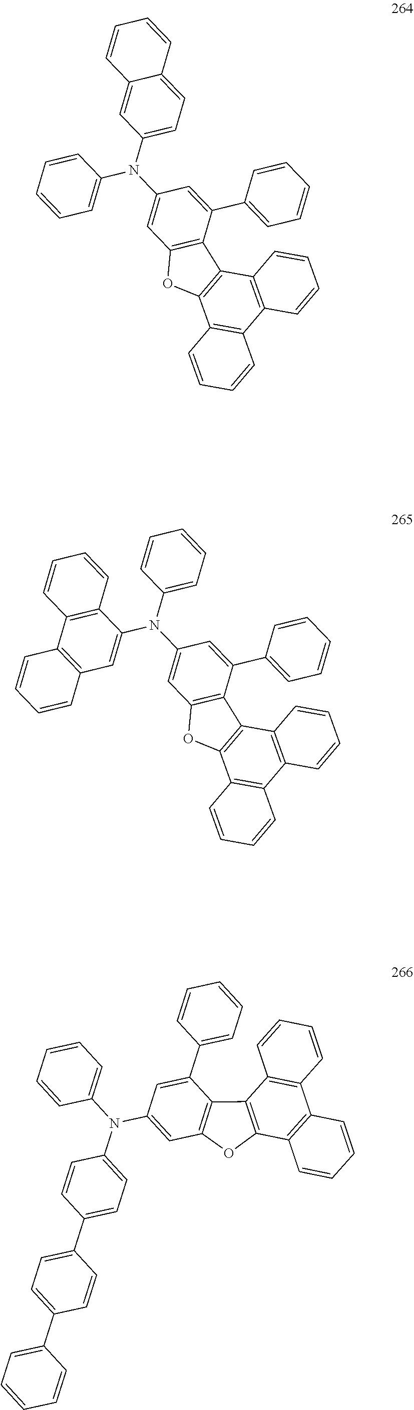 [SMILES: c1ccc(-c2cc(N(c3ccccc3)c3ccc4ccccc4c3)cc3oc4c5ccccc5c5ccccc5c4c23)cc1 ; c1ccc(-c2cc(N(c3ccccc3)c3cc4ccccc4c4ccccc34)cc3oc4c5ccccc5c5ccccc5c4c23)cc1 ; c1ccc(-c2ccc(-c3ccc(N(c4ccccc4)c4cc(-c5ccccc5)c5c(c4)oc4c6ccccc6c6ccccc6c45)cc3)cc2)cc1]

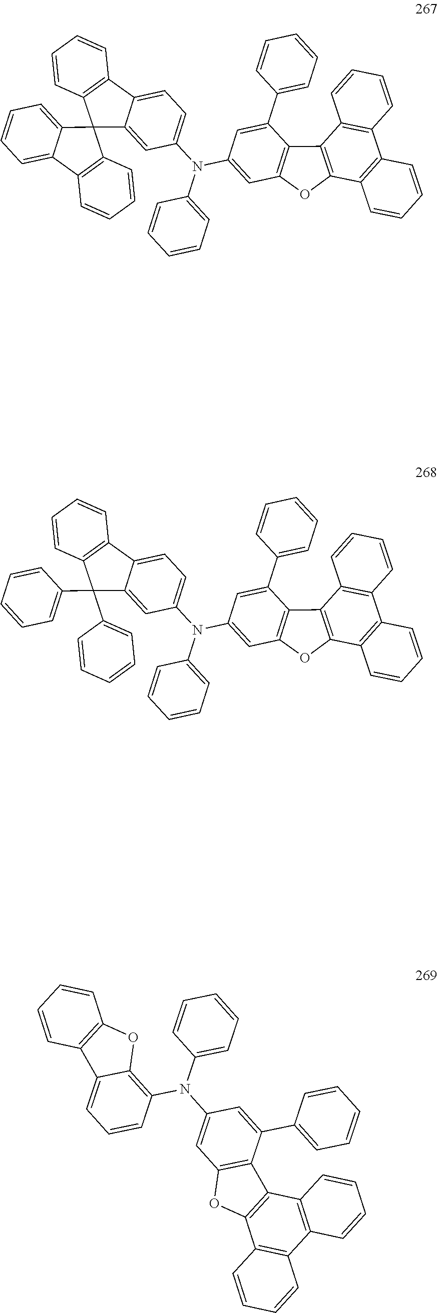 [SMILES: c1ccc(-c2cc(N(c3ccccc3)c3ccc4c(c3)C3(c5ccccc5-c5ccccc53)c3ccccc3-4)cc3oc4c5ccccc5c5ccccc5c4c23)cc1 ; c1ccc(-c2cc(N(c3ccccc3)c3ccc4c(c3)C(c3ccccc3)(c3ccccc3)c3ccccc3-4)cc3oc4c5ccccc5c5ccccc5c4c23)cc1 ; c1ccc(-c2cc(N(c3ccccc3)c3cccc4c3oc3ccccc34)cc3oc4c5ccccc5c5ccccc5c4c23)cc1]

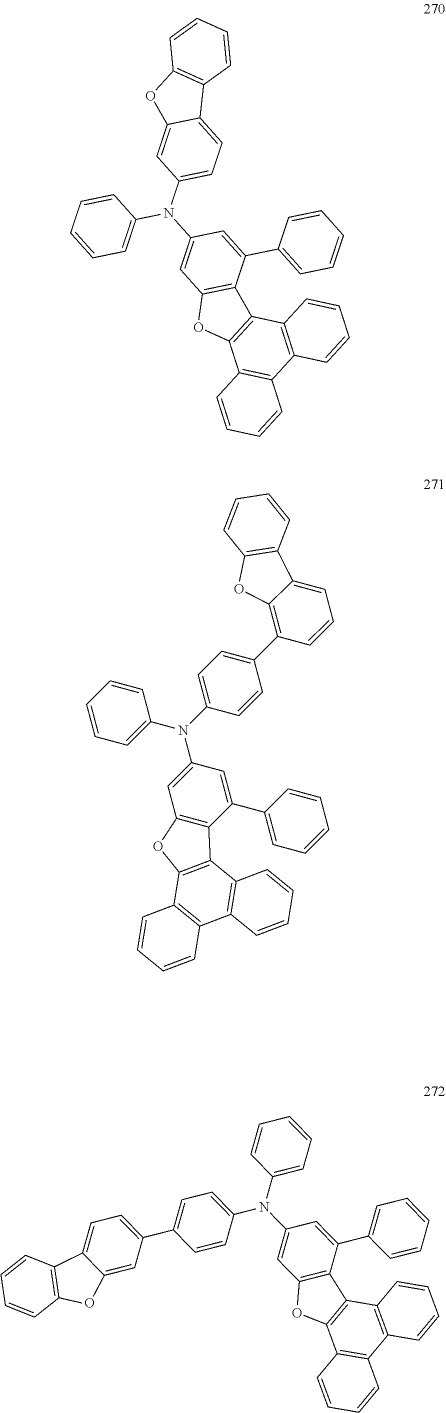 [SMILES: c1ccc(-c2cc(N(c3ccccc3)c3ccc4c(c3)oc3ccccc34)cc3oc4c5ccccc5c5ccccc5c4c23)cc1 ; c1ccc(-c2cc(N(c3ccccc3)c3ccc(-c4cccc5c4oc4ccccc45)cc3)cc3oc4c5ccccc5c5ccccc5c4c23)cc1 ; c1ccc(-c2cc(N(c3ccccc3)c3ccc(-c4ccc5c(c4)oc4ccccc45)cc3)cc3oc4c5ccccc5c5ccccc5c4c23)cc1]

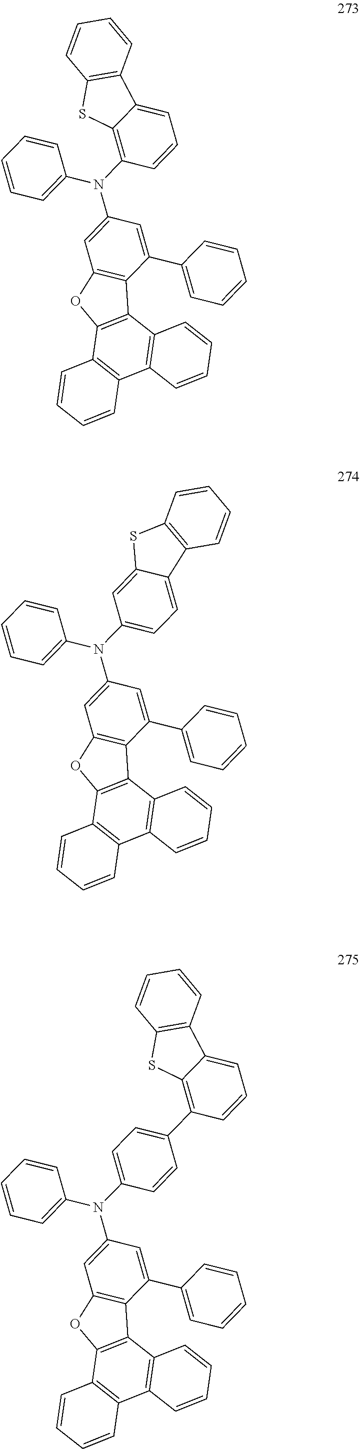 [SMILES: c1ccc(-c2cc(N(c3ccccc3)c3cccc4c3sc3ccccc34)cc3oc4c5ccccc5c5ccccc5c4c23)cc1 ; c1ccc(-c2cc(N(c3ccccc3)c3ccc4c(c3)sc3ccccc34)cc3oc4c5ccccc5c5ccccc5c4c23)cc1 ; c1ccc(-c2cc(N(c3ccccc3)c3ccc(-c4cccc5c4sc4ccccc45)cc3)cc3oc4c5ccccc5c5ccccc5c4c23)cc1]

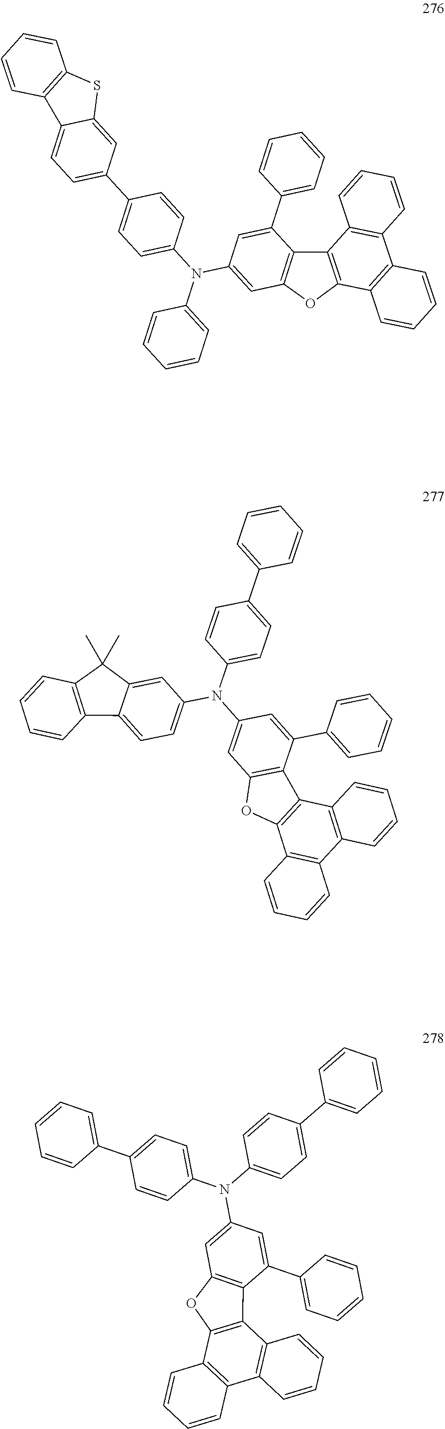 [SMILES: c1ccc(-c2cc(N(c3ccccc3)c3ccc(-c4ccc5c(c4)sc4ccccc45)cc3)cc3oc4c5ccccc5c5ccccc5c4c23)cc1 ; CC1(C)c2ccccc2-c2ccc(N(c3ccc(-c4ccccc4)cc3)c3cc(-c4ccccc4)c4c(c3)oc3c5ccccc5c5ccccc5c34)cc21 ; c1ccc(-c2ccc(N(c3ccc(-c4ccccc4)cc3)c3cc(-c4ccccc4)c4c(c3)oc3c5ccccc5c5ccccc5c34)cc2)cc1]

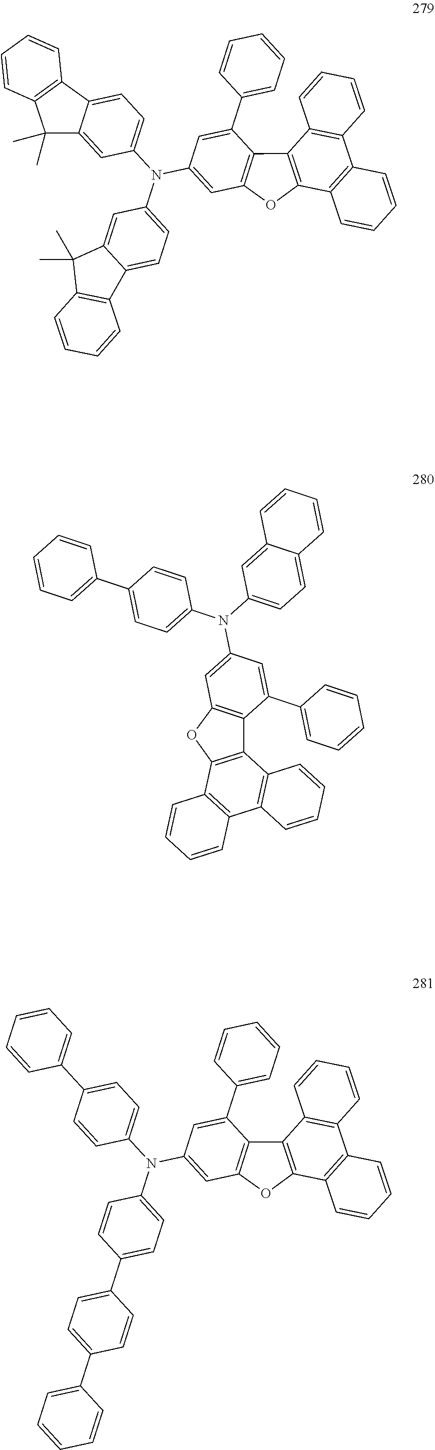 [SMILES: CC1(C)c2ccccc2-c2ccc(N(c3ccc4c(c3)C(C)(C)c3ccccc3-4)c3cc(-c4ccccc4)c4c(c3)oc3c5ccccc5c5ccccc5c34)cc21 ; c1ccc(-c2ccc(N(c3ccc4ccccc4c3)c3cc(-c4ccccc4)c4c(c3)oc3c5ccccc5c5ccccc5c34)cc2)cc1 ; c1ccc(-c2ccc(-c3ccc(N(c4ccc(-c5ccccc5)cc4)c4cc(-c5ccccc5)c5c(c4)oc4c6ccccc6c6ccccc6c45)cc3)cc2)cc1]

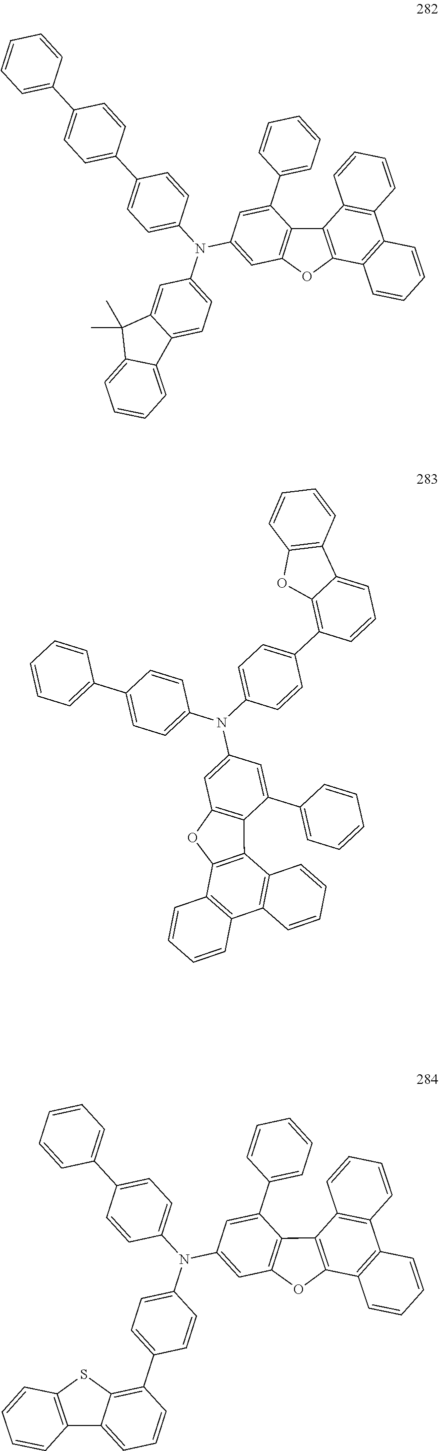 [SMILES: CC1(C)c2ccccc2-c2ccc(N(c3ccc(-c4ccc(-c5ccccc5)cc4)cc3)c3cc(-c4ccccc4)c4c(c3)oc3c5ccccc5c5ccccc5c34)cc21 ; c1ccc(-c2ccc(N(c3ccc(-c4cccc5c4oc4ccccc45)cc3)c3cc(-c4ccccc4)c4c(c3)oc3c5ccccc5c5ccccc5c34)cc2)cc1 ; c1ccc(-c2ccc(N(c3ccc(-c4cccc5c4sc4ccccc45)cc3)c3cc(-c4ccccc4)c4c(c3)oc3c5ccccc5c5ccccc5c34)cc2)cc1]

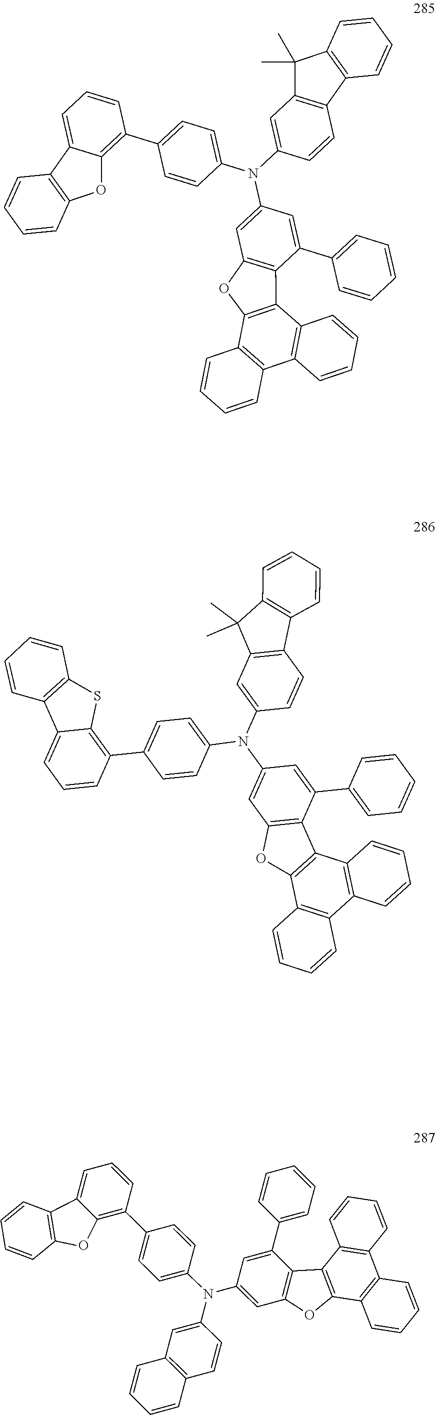 [SMILES: CC1(C)c2ccccc2-c2ccc(N(c3ccc(-c4cccc5c4oc4ccccc45)cc3)c3cc(-c4ccccc4)c4c(c3)oc3c5ccccc5c5ccccc5c34)cc21 ; CC1(C)c2ccccc2-c2ccc(N(c3ccc(-c4cccc5c4sc4ccccc45)cc3)c3cc(-c4ccccc4)c4c(c3)oc3c5ccccc5c5ccccc5c34)cc21 ; c1ccc(-c2cc(N(c3ccc(-c4cccc5c4oc4ccccc45)cc3)c3ccc4ccccc4c3)cc3oc4c5ccccc5c5ccccc5c4c23)cc1]

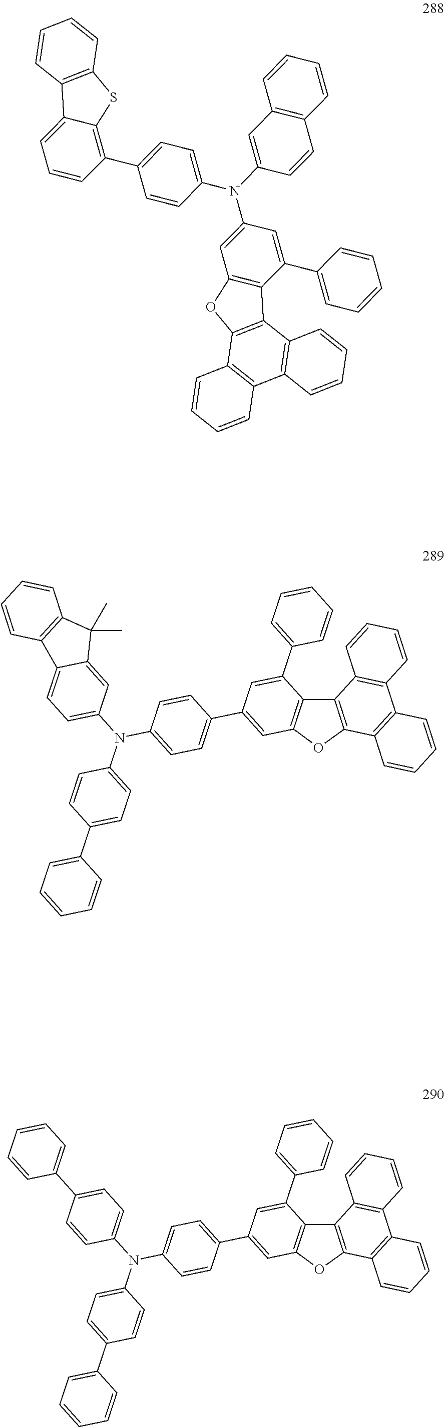 [SMILES: c1ccc(-c2cc(N(c3ccc(-c4cccc5c4sc4ccccc45)cc3)c3ccc4ccccc4c3)cc3oc4c5ccccc5c5ccccc5c4c23)cc1 ; CC1(C)c2ccccc2-c2ccc(N(c3ccc(-c4ccccc4)cc3)c3ccc(-c4cc(-c5ccccc5)c5c(c4)oc4c6ccccc6c6ccccc6c45)cc3)cc21 ; c1ccc(-c2ccc(N(c3ccc(-c4ccccc4)cc3)c3ccc(-c4cc(-c5ccccc5)c5c(c4)oc4c6ccccc6c6ccccc6c45)cc3)cc2)cc1]

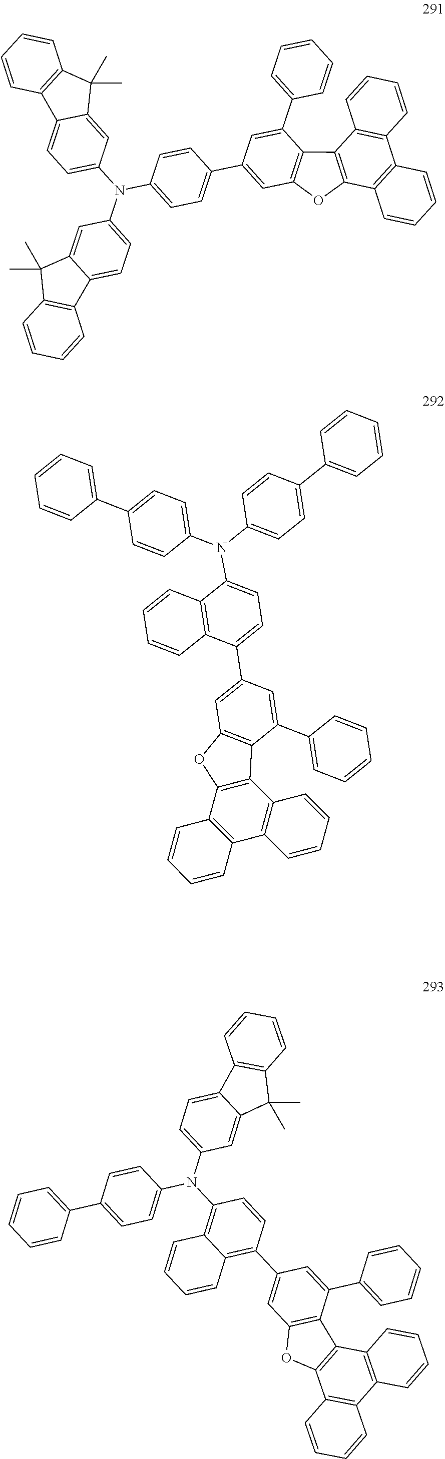 [SMILES: CC1(C)c2ccccc2-c2ccc(N(c3ccc(-c4cc(-c5ccccc5)c5c(c4)oc4c6ccccc6c6ccccc6c45)cc3)c3ccc4c(c3)C(C)(C)c3ccccc3-4)cc21 ; c1ccc(-c2ccc(N(c3ccc(-c4ccccc4)cc3)c3ccc(-c4cc(-c5ccccc5)c5c(c4)oc4c6ccccc6c6ccccc6c45)c4ccccc34)cc2)cc1 ; CC1(C)c2ccccc2-c2ccc(N(c3ccc(-c4ccccc4)cc3)c3ccc(-c4cc(-c5ccccc5)c5c(c4)oc4c6ccccc6c6ccccc6c45)c4ccccc34)cc21]

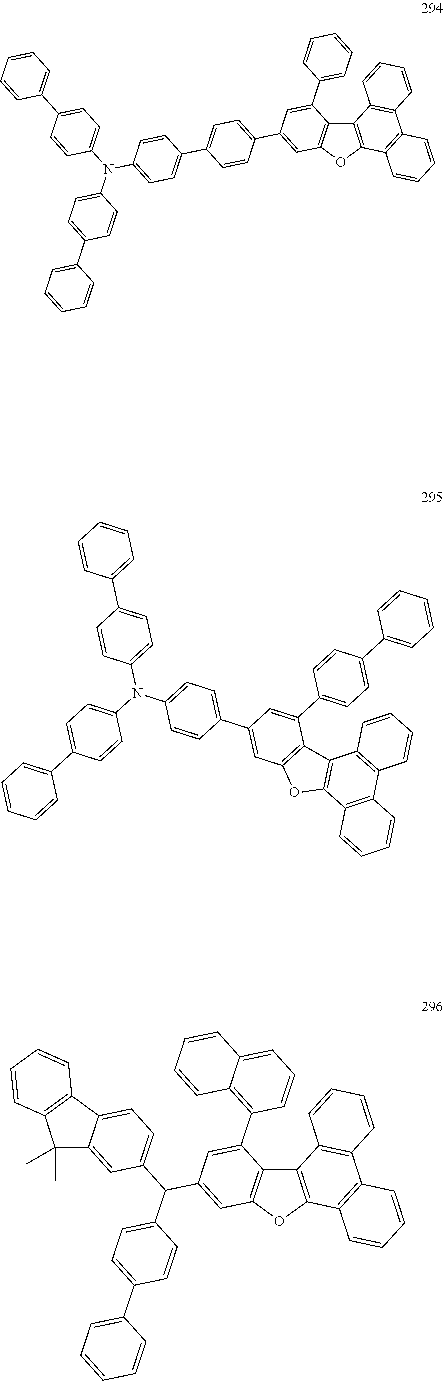 [SMILES: c1ccc(-c2ccc(N(c3ccc(-c4ccccc4)cc3)c3ccc(-c4ccc(-c5cc(-c6ccccc6)c6c(c5)oc5c7ccccc7c7ccccc7c56)cc4)cc3)cc2)cc1 ; c1ccc(-c2ccc(-c3cc(-c4ccc(N(c5ccc(-c6ccccc6)cc5)c5ccc(-c6ccccc6)cc5)cc4)cc4oc5c6ccccc6c6ccccc6c5c34)cc2)cc1 ; CC1(C)c2ccccc2-c2ccc(C(c3ccc(-c4ccccc4)cc3)c3cc(-c4cccc5ccccc45)c4c(c3)oc3c5ccccc5c5ccccc5c34)cc21]

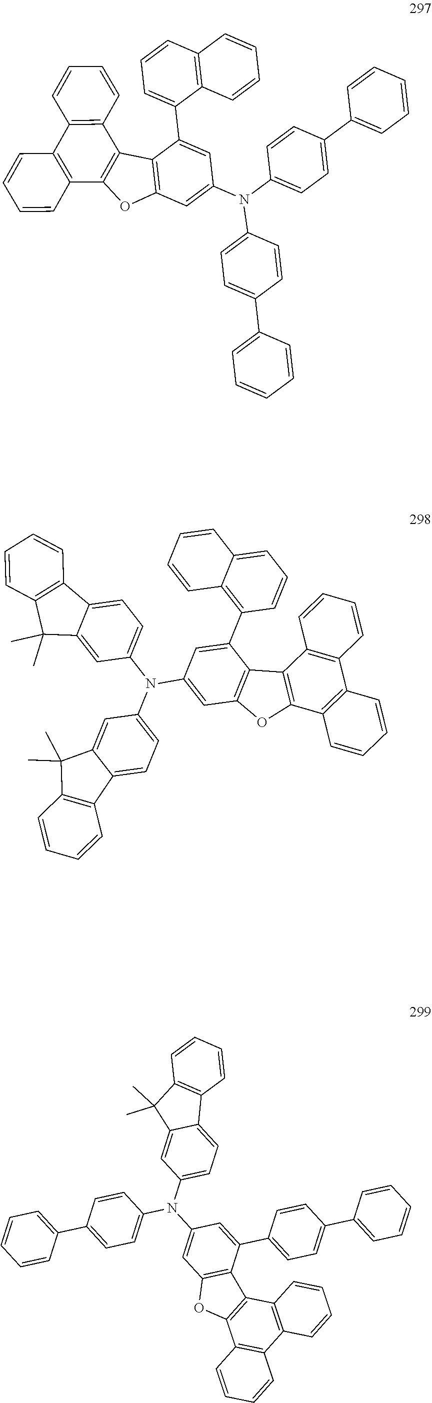 [SMILES: c1ccc(-c2ccc(N(c3ccc(-c4ccccc4)cc3)c3cc(-c4cccc5ccccc45)c4c(c3)oc3c5ccccc5c5ccccc5c34)cc2)cc1 ; CC1(C)c2ccccc2-c2ccc(N(c3ccc4c(c3)C(C)(C)c3ccccc3-4)c3cc(-c4cccc5ccccc45)c4c(c3)oc3c5ccccc5c5ccccc5c34)cc21 ; CC1(C)c2ccccc2-c2ccc(N(c3ccc(-c4ccccc4)cc3)c3cc(-c4ccc(-c5ccccc5)cc4)c4c(c3)oc3c5ccccc5c5ccccc5c34)cc21]

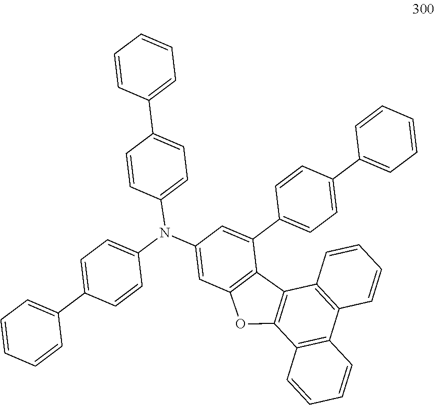 [SMILES: c1ccc(-c2ccc(-c3cc(N(c4ccc(-c5ccccc5)cc4)c4ccc(-c5ccccc5)cc4)cc4oc5c6ccccc6c6ccccc6c5c34)cc2)cc1]

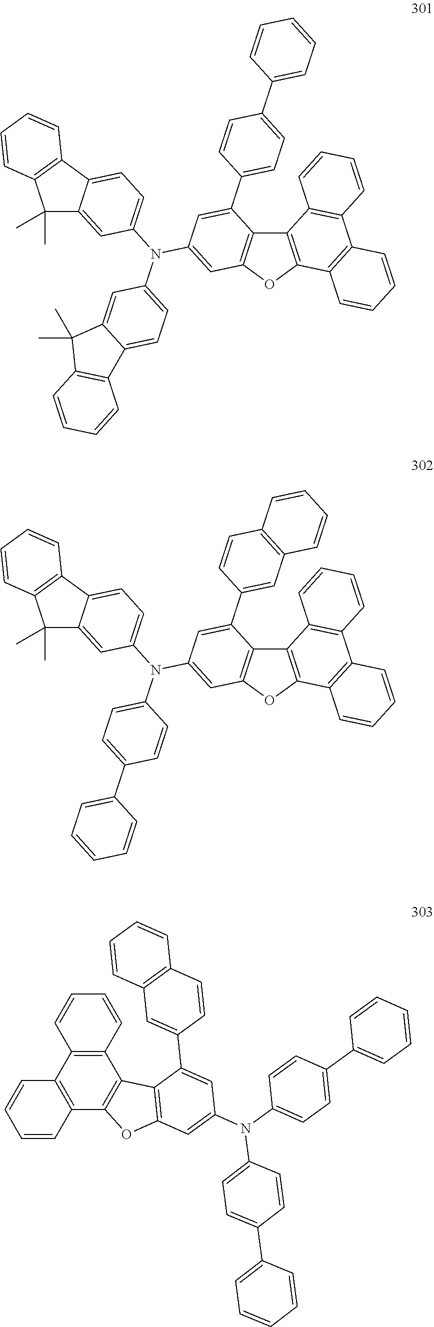 [SMILES: CC1(C)c2ccccc2-c2ccc(N(c3ccc4c(c3)C(C)(C)c3ccccc3-4)c3cc(-c4ccc(-c5ccccc5)cc4)c4c(c3)oc3c5ccccc5c5ccccc5c34)cc21 ; CC1(C)c2ccccc2-c2ccc(N(c3ccc(-c4ccccc4)cc3)c3cc(-c4ccc5ccccc5c4)c4c(c3)oc3c5ccccc5c5ccccc5c34)cc21 ; c1ccc(-c2ccc(N(c3ccc(-c4ccccc4)cc3)c3cc(-c4ccc5ccccc5c4)c4c(c3)oc3c5ccccc5c5ccccc5c34)cc2)cc1]

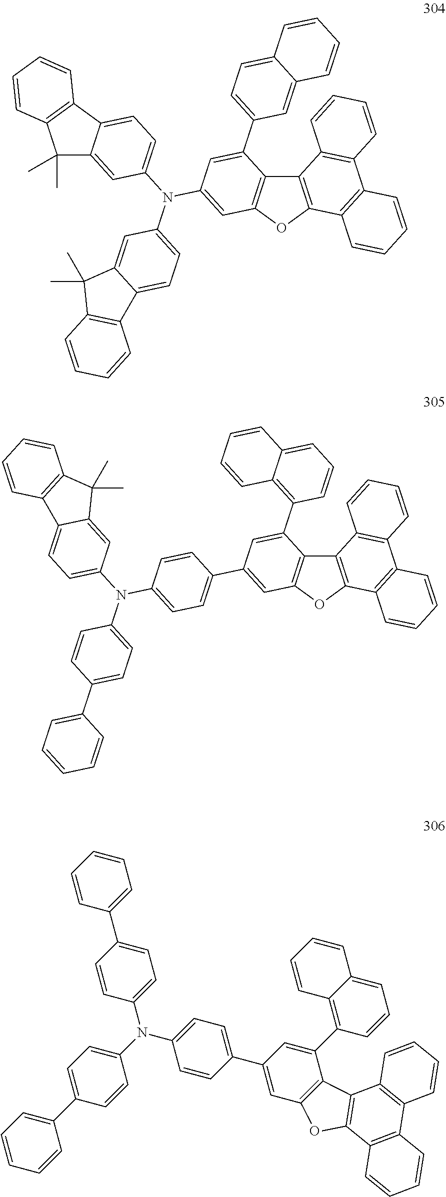 [SMILES: CC1(C)c2ccccc2-c2ccc(N(c3ccc4c(c3)C(C)(C)c3ccccc3-4)c3cc(-c4ccc5ccccc5c4)c4c(c3)oc3c5ccccc5c5ccccc5c34)cc21 ; CC1(C)c2ccccc2-c2ccc(N(c3ccc(-c4ccccc4)cc3)c3ccc(-c4cc(-c5cccc6ccccc56)c5c(c4)oc4c6ccccc6c6ccccc6c45)cc3)cc21 ; c1ccc(-c2ccc(N(c3ccc(-c4ccccc4)cc3)c3ccc(-c4cc(-c5cccc6ccccc56)c5c(c4)oc4c6ccccc6c6ccccc6c45)cc3)cc2)cc1]

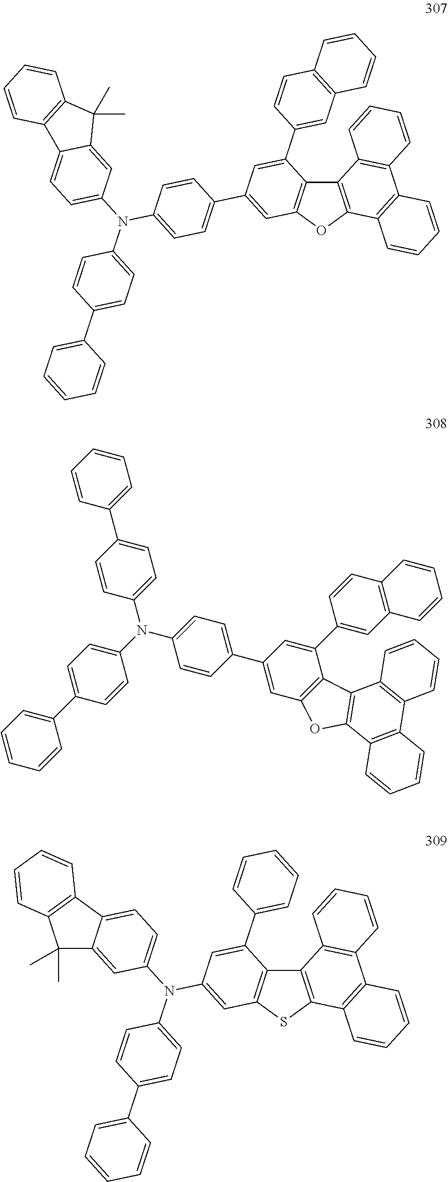 [SMILES: CC1(C)c2ccccc2-c2ccc(N(c3ccc(-c4ccccc4)cc3)c3ccc(-c4cc(-c5ccc6ccccc6c5)c5c(c4)oc4c6ccccc6c6ccccc6c45)cc3)cc21 ; c1ccc(-c2ccc(N(c3ccc(-c4ccccc4)cc3)c3ccc(-c4cc(-c5ccc6ccccc6c5)c5c(c4)oc4c6ccccc6c6ccccc6c45)cc3)cc2)cc1 ; CC1(C)c2ccccc2-c2ccc(N(c3ccc(-c4ccccc4)cc3)c3cc(-c4ccccc4)c4c(c3)sc3c5ccccc5c5ccccc5c34)cc21]

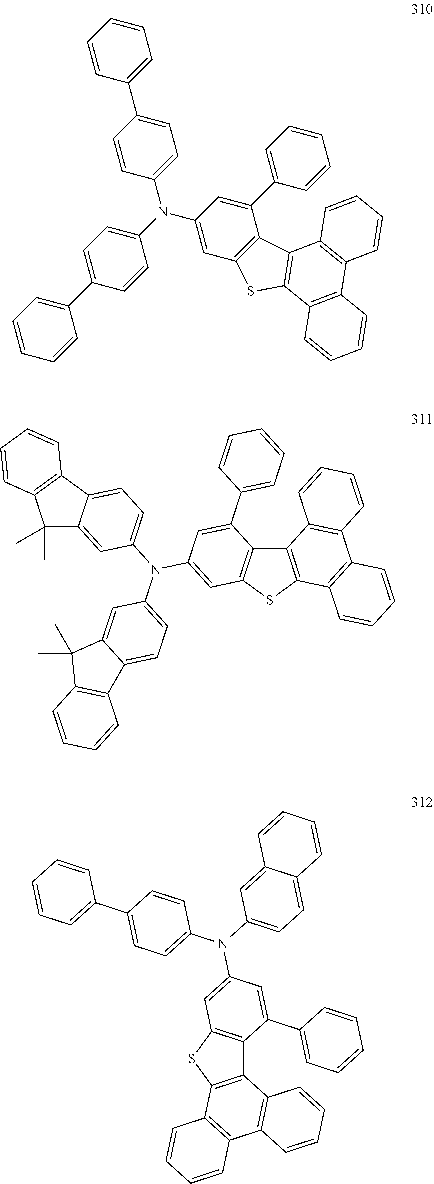 [SMILES: c1ccc(-c2ccc(N(c3ccc(-c4ccccc4)cc3)c3cc(-c4ccccc4)c4c(c3)sc3c5ccccc5c5ccccc5c34)cc2)cc1 ; CC1(C)c2ccccc2-c2ccc(N(c3ccc4c(c3)C(C)(C)c3ccccc3-4)c3cc(-c4ccccc4)c4c(c3)sc3c5ccccc5c5ccccc5c34)cc21 ; c1ccc(-c2ccc(N(c3ccc4ccccc4c3)c3cc(-c4ccccc4)c4c(c3)sc3c5ccccc5c5ccccc5c34)cc2)cc1]

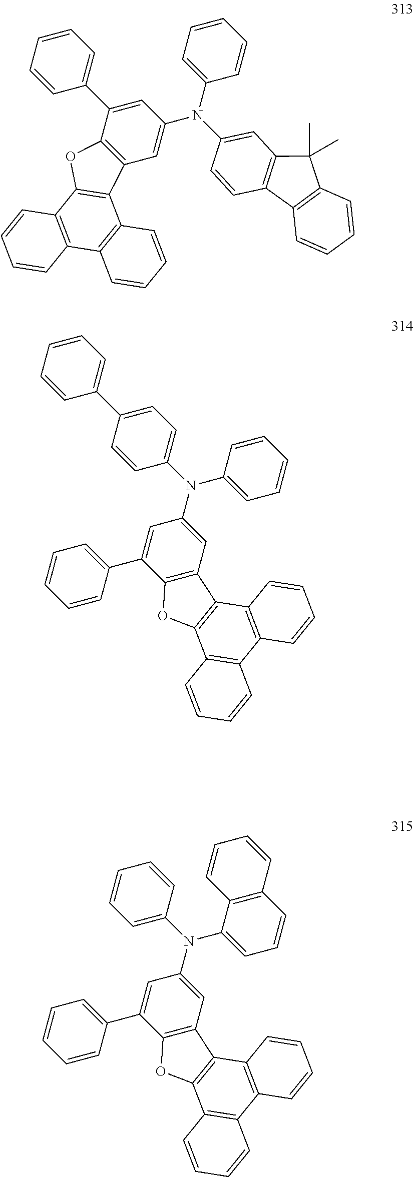 [SMILES: CC1(C)c2ccccc2-c2ccc(N(c3ccccc3)c3cc(-c4ccccc4)c4oc5c6ccccc6c6ccccc6c5c4c3)cc21 ; c1ccc(-c2ccc(N(c3ccccc3)c3cc(-c4ccccc4)c4oc5c6ccccc6c6ccccc6c5c4c3)cc2)cc1 ; c1ccc(-c2cc(N(c3ccccc3)c3cccc4ccccc34)cc3c2oc2c4ccccc4c4ccccc4c32)cc1]

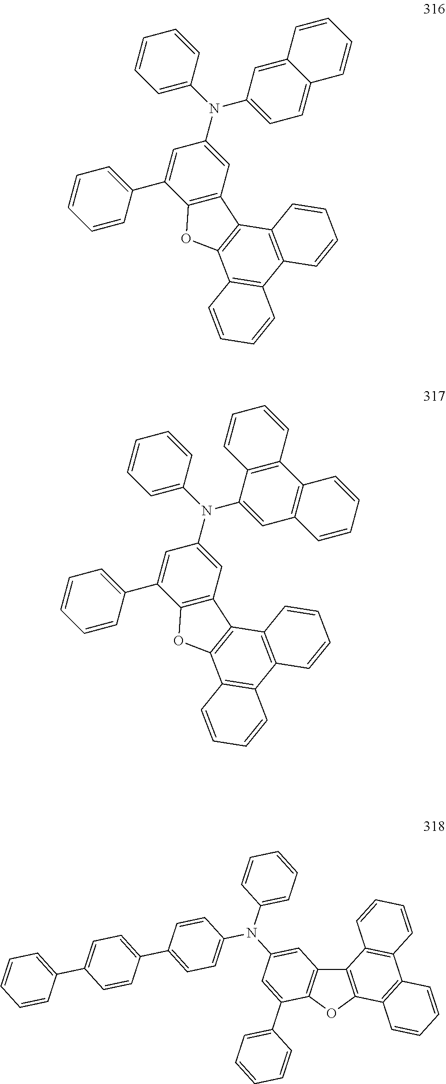 [SMILES: c1ccc(-c2cc(N(c3ccccc3)c3ccc4ccccc4c3)cc3c2oc2c4ccccc4c4ccccc4c32)cc1 ; c1ccc(-c2cc(N(c3ccccc3)c3cc4ccccc4c4ccccc34)cc3c2oc2c4ccccc4c4ccccc4c32)cc1 ; c1ccc(-c2ccc(-c3ccc(N(c4ccccc4)c4cc(-c5ccccc5)c5oc6c7ccccc7c7ccccc7c6c5c4)cc3)cc2)cc1]

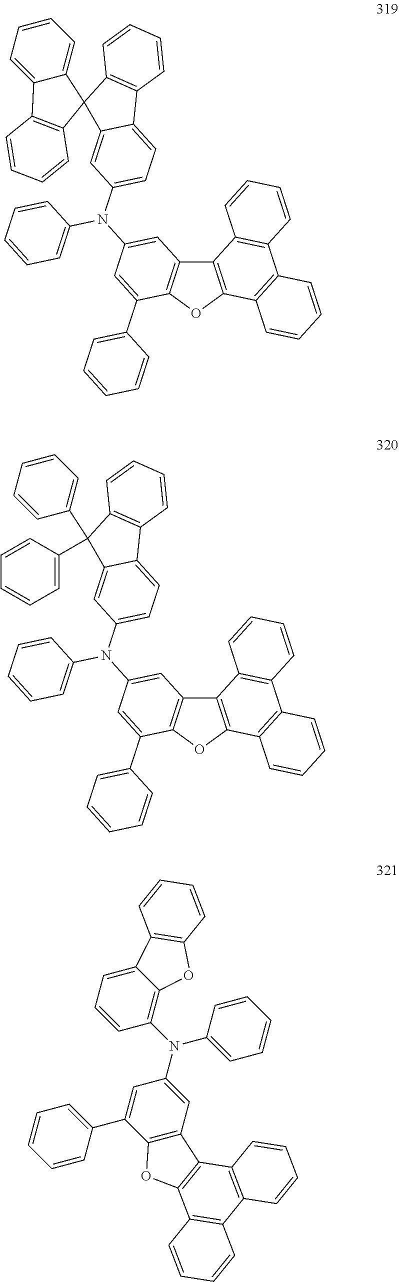 [SMILES: c1ccc(-c2cc(N(c3ccccc3)c3ccc4c(c3)C3(c5ccccc5-c5ccccc53)c3ccccc3-4)cc3c2oc2c4ccccc4c4ccccc4c32)cc1 ; c1ccc(-c2cc(N(c3ccccc3)c3ccc4c(c3)C(c3ccccc3)(c3ccccc3)c3ccccc3-4)cc3c2oc2c4ccccc4c4ccccc4c32)cc1 ; c1ccc(-c2cc(N(c3ccccc3)c3cccc4c3oc3ccccc34)cc3c2oc2c4ccccc4c4ccccc4c32)cc1]

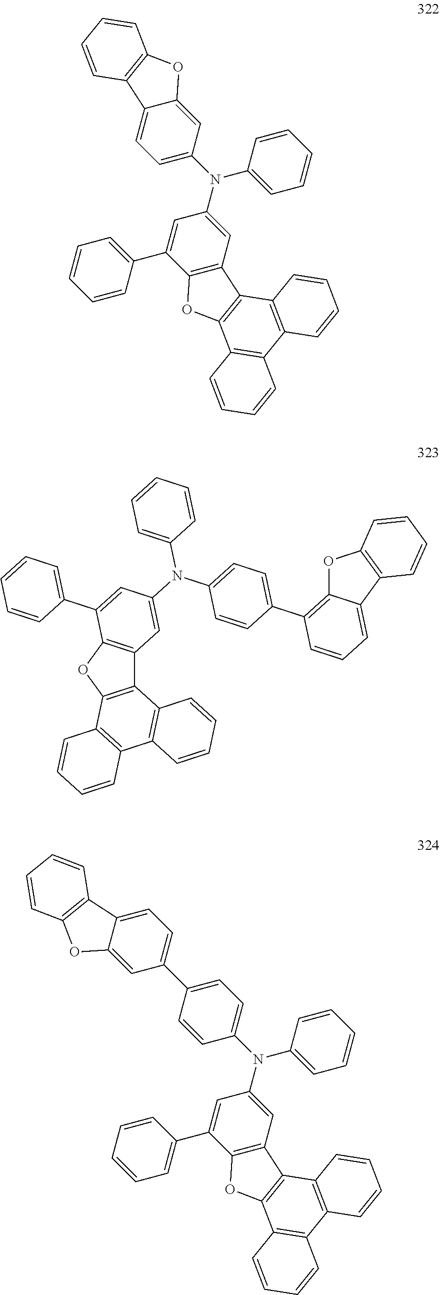 [SMILES: c1ccc(-c2cc(N(c3ccccc3)c3ccc4c(c3)oc3ccccc34)cc3c2oc2c4ccccc4c4ccccc4c32)cc1 ; c1ccc(-c2cc(N(c3ccccc3)c3ccc(-c4cccc5c4oc4ccccc45)cc3)cc3c2oc2c4ccccc4c4ccccc4c32)cc1 ; c1ccc(-c2cc(N(c3ccccc3)c3ccc(-c4ccc5c(c4)oc4ccccc45)cc3)cc3c2oc2c4ccccc4c4ccccc4c32)cc1]

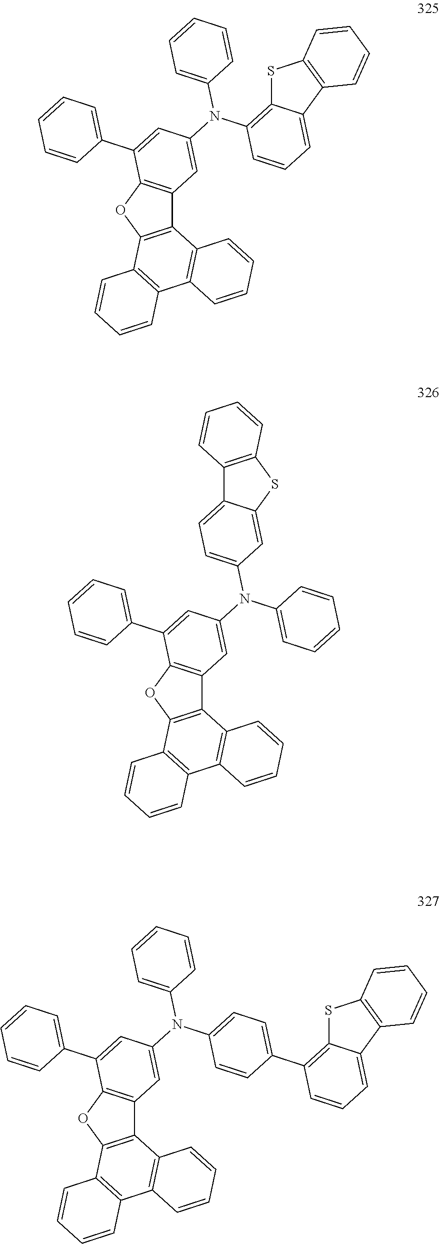 [SMILES: c1ccc(-c2cc(N(c3ccccc3)c3cccc4c3sc3ccccc34)cc3c2oc2c4ccccc4c4ccccc4c32)cc1 ; c1ccc(-c2cc(N(c3ccccc3)c3ccc4c(c3)sc3ccccc34)cc3c2oc2c4ccccc4c4ccccc4c32)cc1 ; c1ccc(-c2cc(N(c3ccccc3)c3ccc(-c4cccc5c4sc4ccccc45)cc3)cc3c2oc2c4ccccc4c4ccccc4c32)cc1]

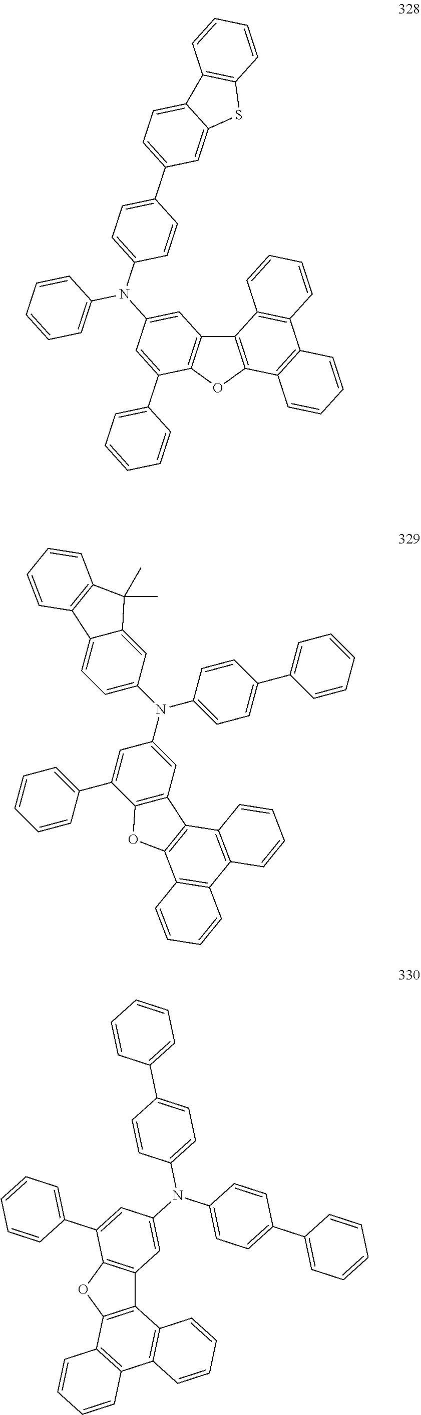 [SMILES: c1ccc(-c2cc(N(c3ccccc3)c3ccc(-c4ccc5c(c4)sc4ccccc45)cc3)cc3c2oc2c4ccccc4c4ccccc4c32)cc1 ; CC1(C)c2ccccc2-c2ccc(N(c3ccc(-c4ccccc4)cc3)c3cc(-c4ccccc4)c4oc5c6ccccc6c6ccccc6c5c4c3)cc21 ; c1ccc(-c2ccc(N(c3ccc(-c4ccccc4)cc3)c3cc(-c4ccccc4)c4oc5c6ccccc6c6ccccc6c5c4c3)cc2)cc1]

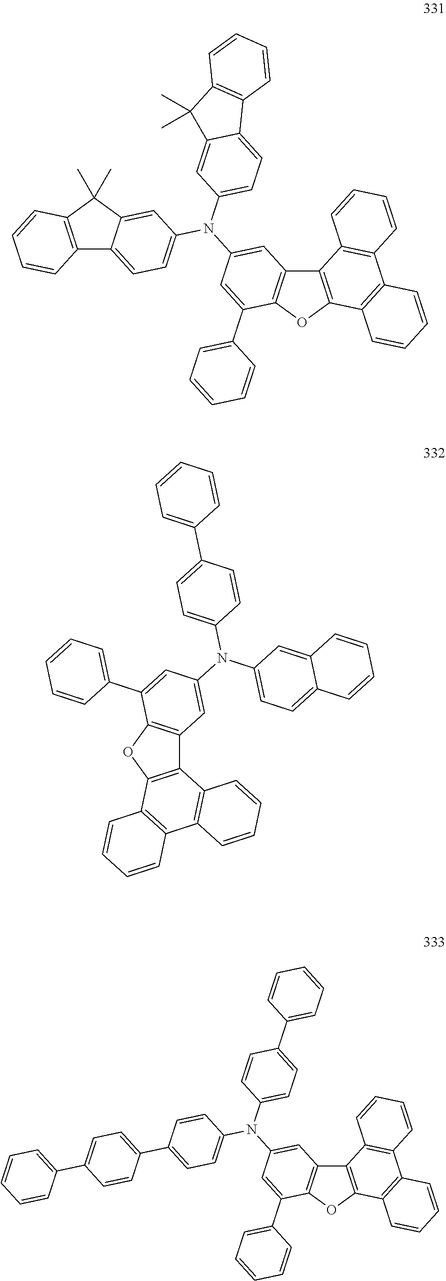 [SMILES: CC1(C)c2ccccc2-c2ccc(N(c3ccc4c(c3)C(C)(C)c3ccccc3-4)c3cc(-c4ccccc4)c4oc5c6ccccc6c6ccccc6c5c4c3)cc21 ; c1ccc(-c2ccc(N(c3ccc4ccccc4c3)c3cc(-c4ccccc4)c4oc5c6ccccc6c6ccccc6c5c4c3)cc2)cc1 ; c1ccc(-c2ccc(-c3ccc(N(c4ccc(-c5ccccc5)cc4)c4cc(-c5ccccc5)c5oc6c7ccccc7c7ccccc7c6c5c4)cc3)cc2)cc1]

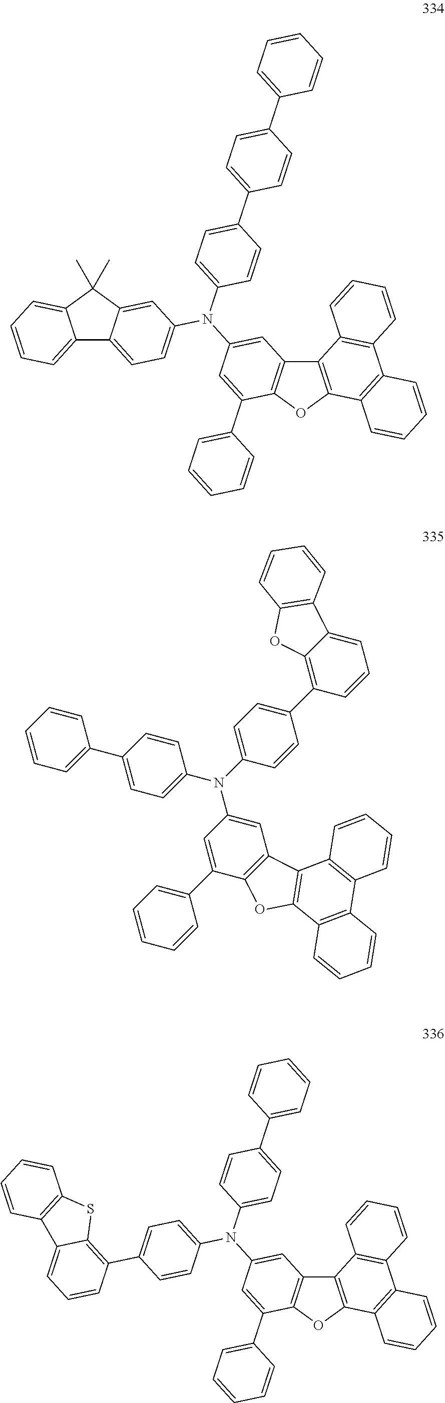 [SMILES: CC1(C)c2ccccc2-c2ccc(N(c3ccc(-c4ccc(-c5ccccc5)cc4)cc3)c3cc(-c4ccccc4)c4oc5c6ccccc6c6ccccc6c5c4c3)cc21 ; c1ccc(-c2ccc(N(c3ccc(-c4cccc5c4oc4ccccc45)cc3)c3cc(-c4ccccc4)c4oc5c6ccccc6c6ccccc6c5c4c3)cc2)cc1 ; c1ccc(-c2ccc(N(c3ccc(-c4cccc5c4sc4ccccc45)cc3)c3cc(-c4ccccc4)c4oc5c6ccccc6c6ccccc6c5c4c3)cc2)cc1]

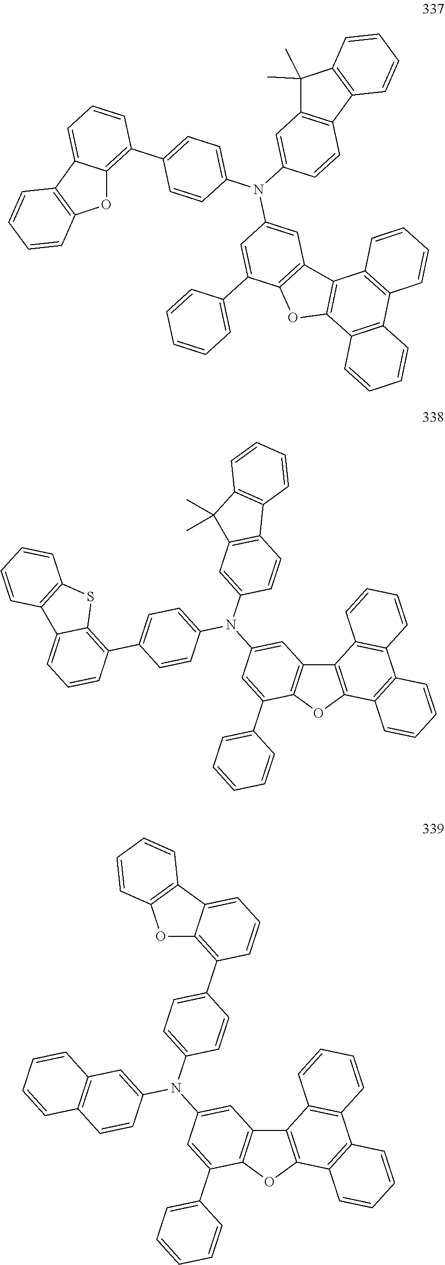 [SMILES: CC1(C)c2ccccc2-c2ccc(N(c3ccc(-c4cccc5c4oc4ccccc45)cc3)c3cc(-c4ccccc4)c4oc5c6ccccc6c6ccccc6c5c4c3)cc21 ; CC1(C)c2ccccc2-c2ccc(N(c3ccc(-c4cccc5c4sc4ccccc45)cc3)c3cc(-c4ccccc4)c4oc5c6ccccc6c6ccccc6c5c4c3)cc21 ; c1ccc(-c2cc(N(c3ccc(-c4cccc5c4oc4ccccc45)cc3)c3ccc4ccccc4c3)cc3c2oc2c4ccccc4c4ccccc4c32)cc1]

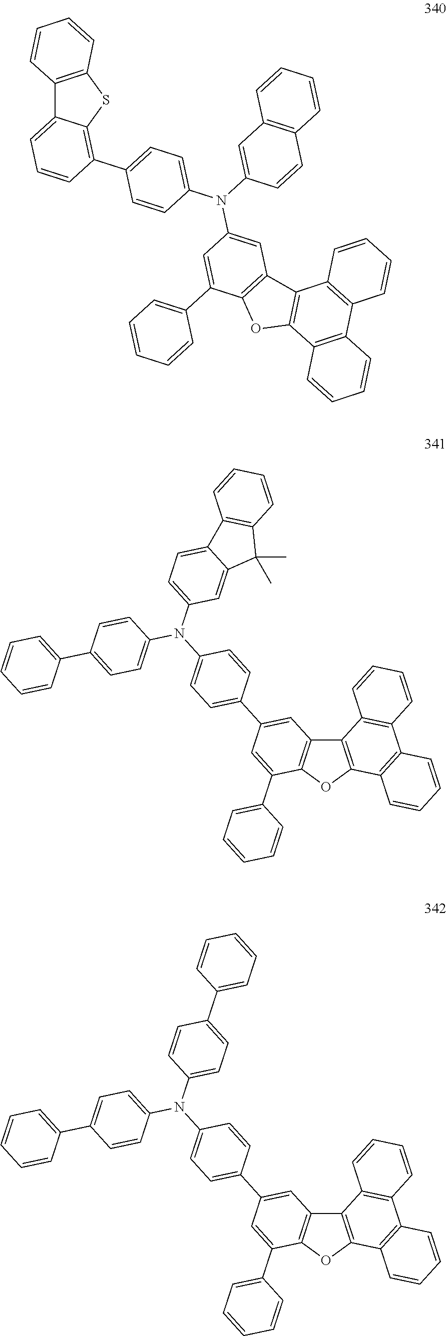 [SMILES: c1ccc(-c2cc(N(c3ccc(-c4cccc5c4sc4ccccc45)cc3)c3ccc4ccccc4c3)cc3c2oc2c4ccccc4c4ccccc4c32)cc1 ; CC1(C)c2ccccc2-c2ccc(N(c3ccc(-c4ccccc4)cc3)c3ccc(-c4cc(-c5ccccc5)c5oc6c7ccccc7c7ccccc7c6c5c4)cc3)cc21 ; c1ccc(-c2ccc(N(c3ccc(-c4ccccc4)cc3)c3ccc(-c4cc(-c5ccccc5)c5oc6c7ccccc7c7ccccc7c6c5c4)cc3)cc2)cc1]

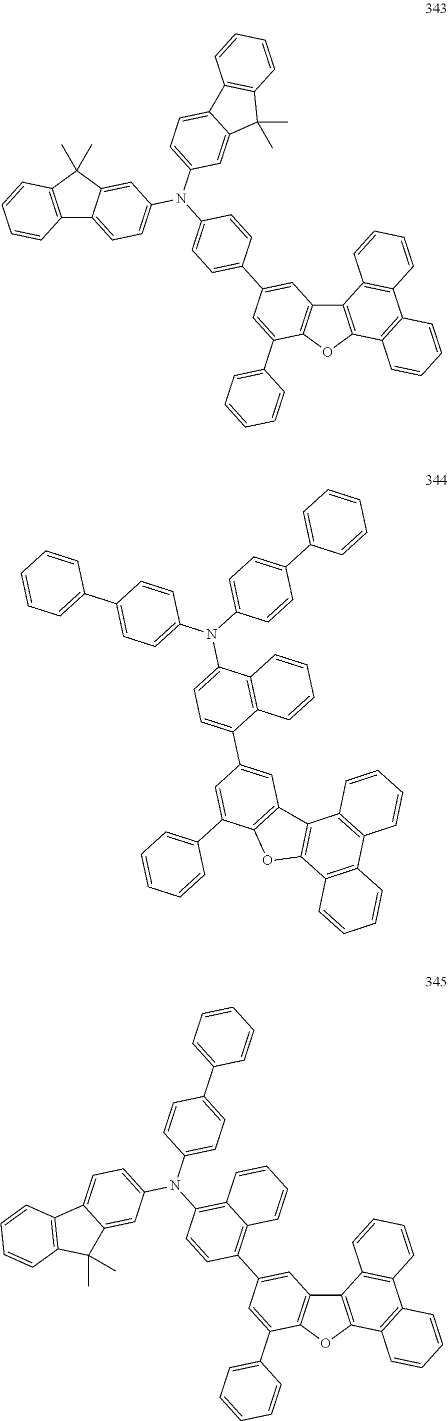 [SMILES: CC1(C)c2ccccc2-c2ccc(N(c3ccc(-c4cc(-c5ccccc5)c5oc6c7ccccc7c7ccccc7c6c5c4)cc3)c3ccc4c(c3)C(C)(C)c3ccccc3-4)cc21 ; c1ccc(-c2ccc(N(c3ccc(-c4ccccc4)cc3)c3ccc(-c4cc(-c5ccccc5)c5oc6c7ccccc7c7ccccc7c6c5c4)c4ccccc34)cc2)cc1 ; CC1(C)c2ccccc2-c2ccc(N(c3ccc(-c4ccccc4)cc3)c3ccc(-c4cc(-c5ccccc5)c5oc6c7ccccc7c7ccccc7c6c5c4)c4ccccc34)cc21]

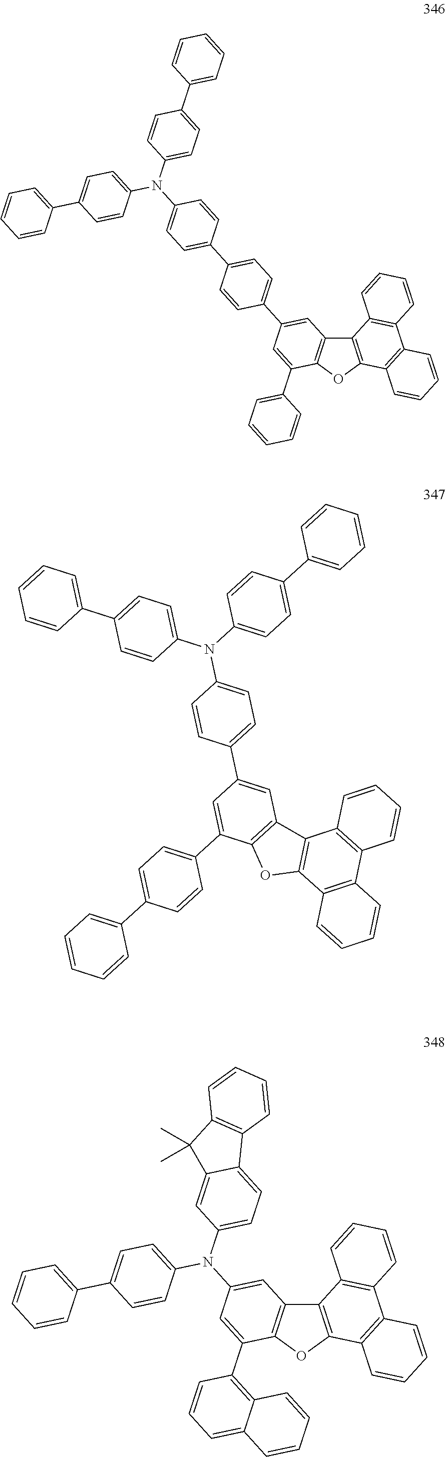 [SMILES: c1ccc(-c2ccc(N(c3ccc(-c4ccccc4)cc3)c3ccc(-c4ccc(-c5cc(-c6ccccc6)c6oc7c8ccccc8c8ccccc8c7c6c5)cc4)cc3)cc2)cc1 ; c1ccc(-c2ccc(-c3cc(-c4ccc(N(c5ccc(-c6ccccc6)cc5)c5ccc(-c6ccccc6)cc5)cc4)cc4c3oc3c5ccccc5c5ccccc5c43)cc2)cc1 ; CC1(C)c2ccccc2-c2ccc(N(c3ccc(-c4ccccc4)cc3)c3cc(-c4cccc5ccccc45)c4oc5c6ccccc6c6ccccc6c5c4c3)cc21]

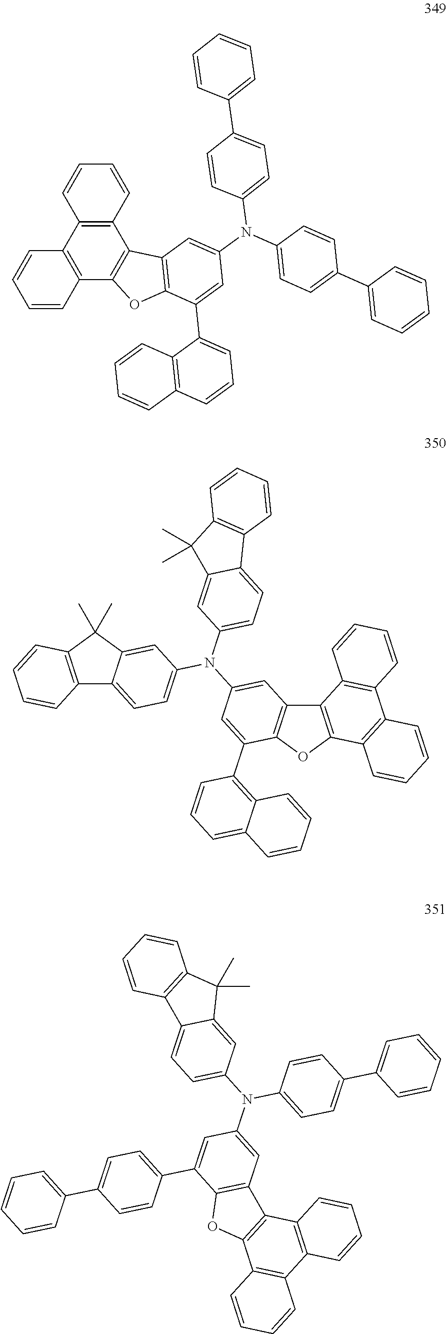 [SMILES: c1ccc(-c2ccc(N(c3ccc(-c4ccccc4)cc3)c3cc(-c4cccc5ccccc45)c4oc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4cccc5ccccc45)c4oc5c6ccccc6c6ccccc6c5c4c3)cc21 ; CC1(C)c2ccccc2-c2ccc(N(c3ccc(-c4ccccc4)cc3)c3cc(-c4ccc(-c5ccccc5)cc4)c4oc5c6ccccc6c6ccccc6c5c4c3)cc21]

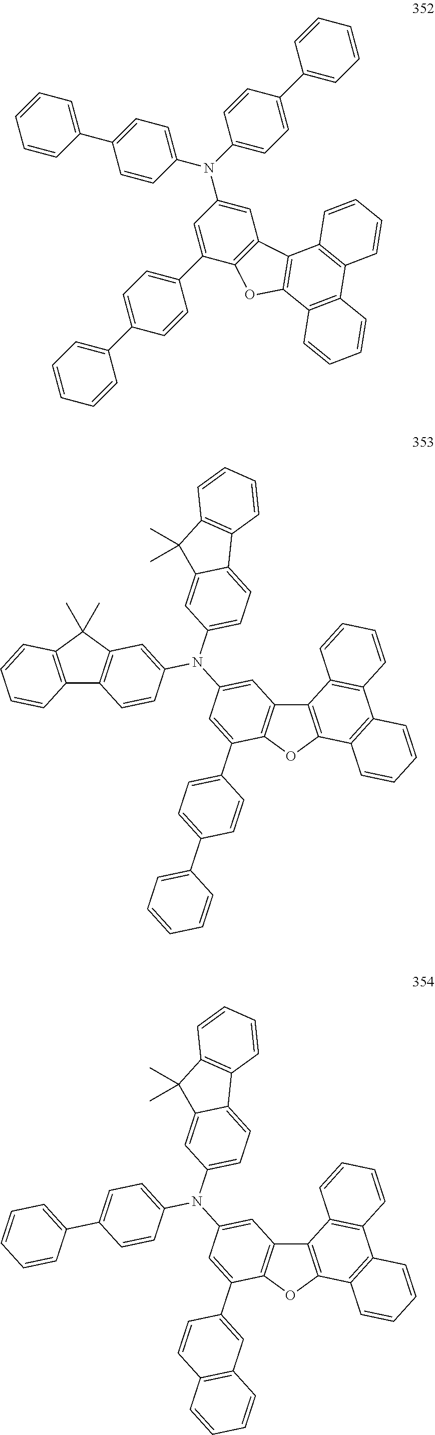 [SMILES: c1ccc(-c2ccc(-c3cc(N(c4ccc(-c5ccccc5)cc4)c4ccc(-c5ccccc5)cc4)cc4c3oc3c5ccccc5c5ccccc5c43)cc2)cc1 ; CC1(C)c2ccccc2-c2ccc(N(c3ccc4c(c3)C(C)(C)c3ccccc3-4)c3cc(-c4ccc(-c5ccccc5)cc4)c4oc5c6ccccc6c6ccccc6c5c4c3)cc21 ; CC1(C)c2ccccc2-c2ccc(N(c3ccc(-c4ccccc4)cc3)c3cc(-c4ccc5ccccc5c4)c4oc5c6ccccc6c6ccccc6c5c4c3)cc21]

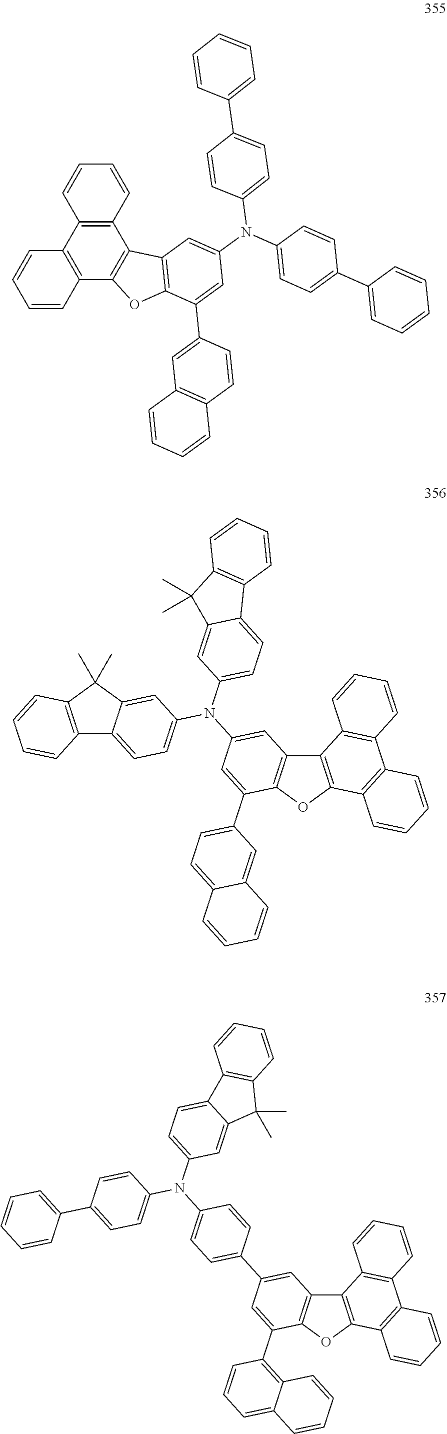 [SMILES: c1ccc(-c2ccc(N(c3ccc(-c4ccccc4)cc3)c3cc(-c4ccc5ccccc5c4)c4oc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccc5ccccc5c4)c4oc5c6ccccc6c6ccccc6c5c4c3)cc21 ; CC1(C)c2ccccc2-c2ccc(N(c3ccc(-c4ccccc4)cc3)c3ccc(-c4cc(-c5cccc6ccccc56)c5oc6c7ccccc7c7ccccc7c6c5c4)cc3)cc21]

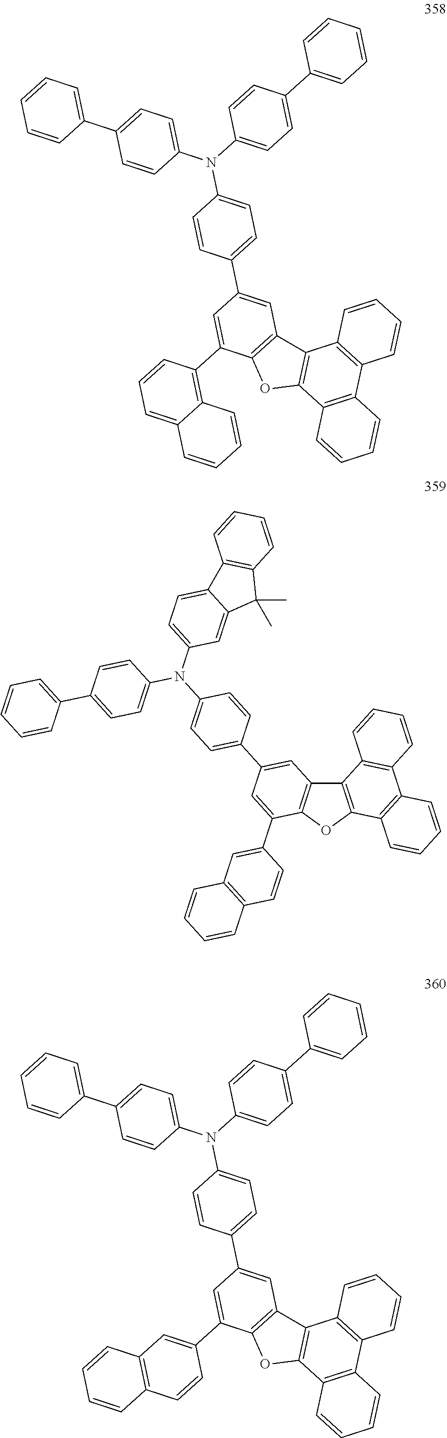 [SMILES: c1ccc(-c2ccc(N(c3ccc(-c4ccccc4)cc3)c3ccc(-c4cc(-c5cccc6ccccc56)c5oc6c7ccccc7c7ccccc7c6c5c4)cc3)cc2)cc1 ; CC1(C)c2ccccc2-c2ccc(N(c3ccc(-c4ccccc4)cc3)c3ccc(-c4cc(-c5ccc6ccccc6c5)c5oc6c7ccccc7c7ccccc7c6c5c4)cc3)cc21 ; c1ccc(-c2ccc(N(c3ccc(-c4ccccc4)cc3)c3ccc(-c4cc(-c5ccc6ccccc6c5)c5oc6c7ccccc7c7ccccc7c6c5c4)cc3)cc2)cc1]

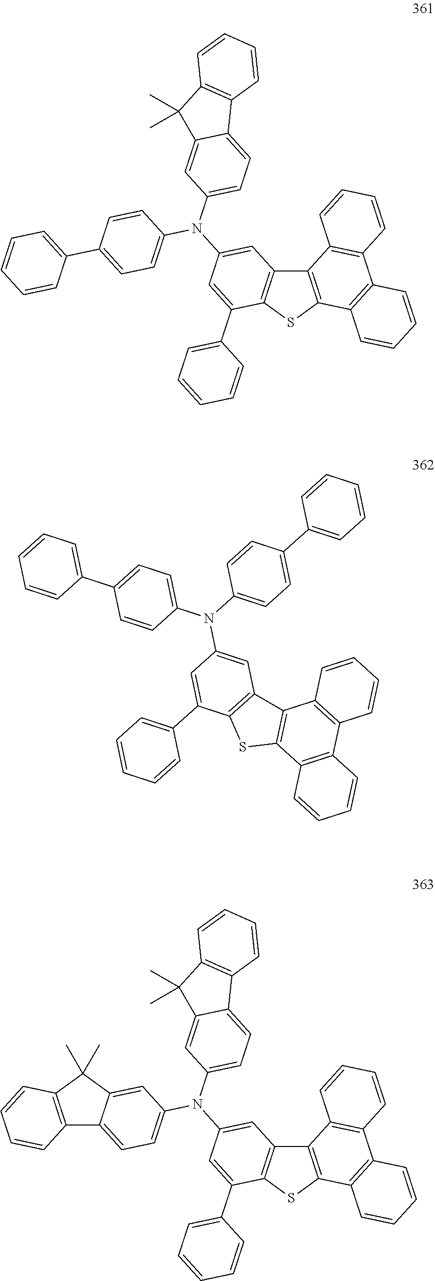 [SMILES: CC1(C)c2ccccc2-c2ccc(N(c3ccc(-c4ccccc4)cc3)c3cc(-c4ccccc4)c4sc5c6ccccc6c6ccccc6c5c4c3)cc21 ; c1ccc(-c2ccc(N(c3ccc(-c4ccccc4)cc3)c3cc(-c4ccccc4)c4sc5c6ccccc6c6ccccc6c5c4c3)cc2)cc1 ; CC1(C)c2ccccc2-c2ccc(N(c3ccc4c(c3)C(C)(C)c3ccccc3-4)c3cc(-c4ccccc4)c4sc5c6ccccc6c6ccccc6c5c4c3)cc21]

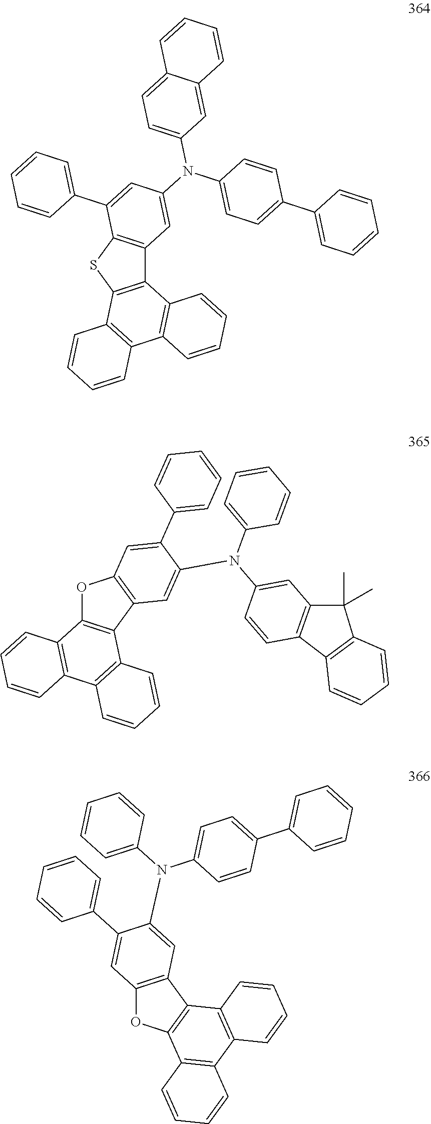 [SMILES: c1ccc(-c2ccc(N(c3ccc4ccccc4c3)c3cc(-c4ccccc4)c4sc5c6ccccc6c6ccccc6c5c4c3)cc2)cc1 ; CC1(C)c2ccccc2-c2ccc(N(c3ccccc3)c3cc4c(cc3-c3ccccc3)oc3c5ccccc5c5ccccc5c43)cc21 ; c1ccc(-c2ccc(N(c3ccccc3)c3cc4c(cc3-c3ccccc3)oc3c5ccccc5c5ccccc5c43)cc2)cc1]

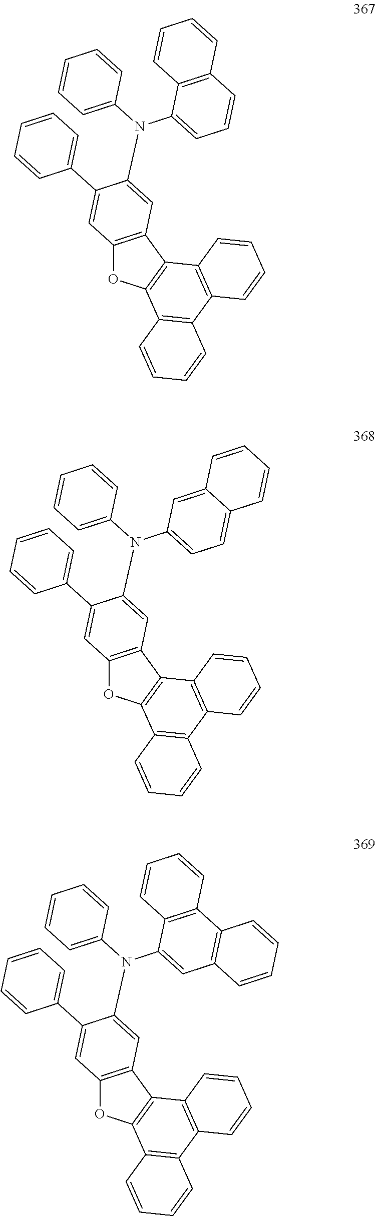 [SMILES: c1ccc(-c2cc3oc4c5ccccc5c5ccccc5c4c3cc2N(c2ccccc2)c2cccc3ccccc23)cc1 ; c1ccc(-c2cc3oc4c5ccccc5c5ccccc5c4c3cc2N(c2ccccc2)c2ccc3ccccc3c2)cc1 ; c1ccc(-c2cc3oc4c5ccccc5c5ccccc5c4c3cc2N(c2ccccc2)c2cc3ccccc3c3ccccc23)cc1]

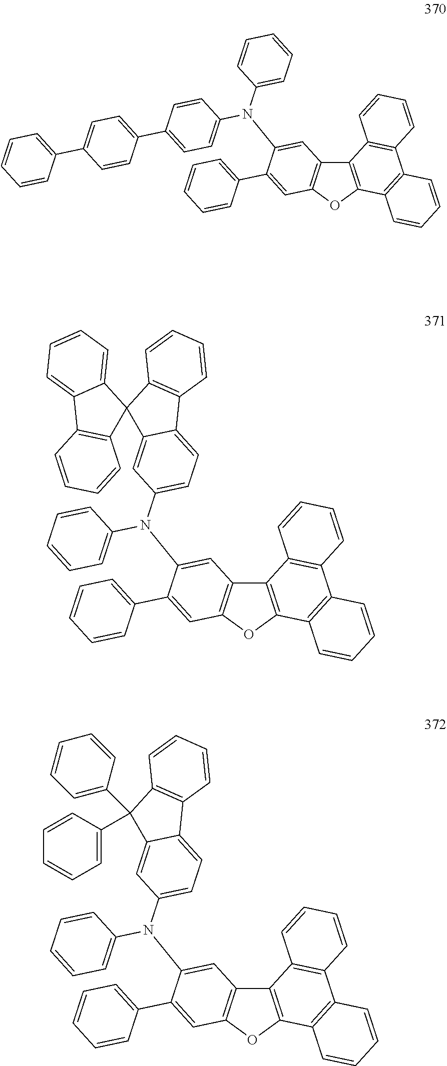 [SMILES: c1ccc(-c2ccc(-c3ccc(N(c4ccccc4)c4cc5c(cc4-c4ccccc4)oc4c6ccccc6c6ccccc6c54)cc3)cc2)cc1 ; c1ccc(-c2cc3oc4c5ccccc5c5ccccc5c4c3cc2N(c2ccccc2)c2ccc3c(c2)C2(c4ccccc4-c4ccccc42)c2ccccc2-3)cc1 ; c1ccc(-c2cc3oc4c5ccccc5c5ccccc5c4c3cc2N(c2ccccc2)c2ccc3c(c2)C(c2ccccc2)(c2ccccc2)c2ccccc2-3)cc1]

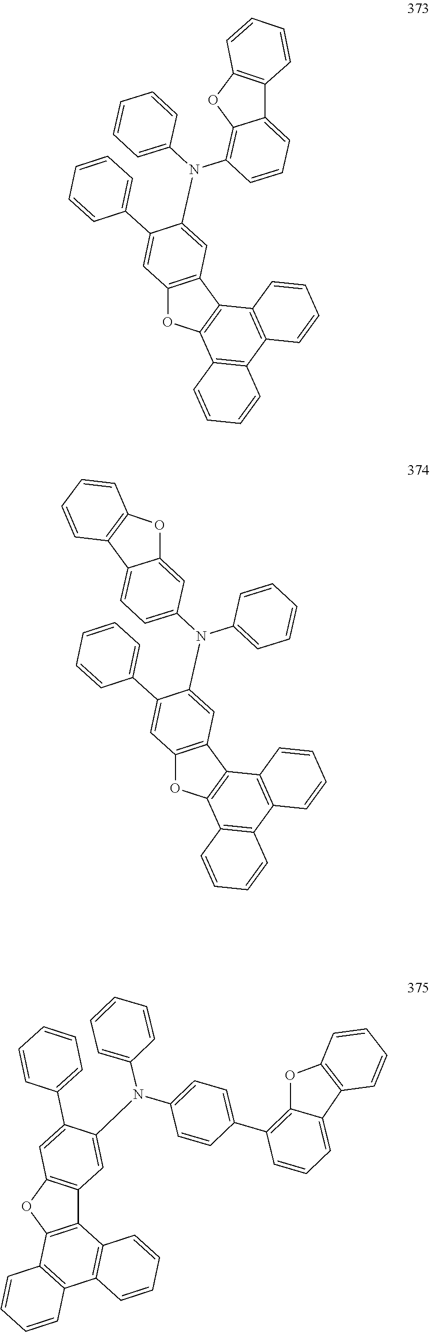 [SMILES: c1ccc(-c2cc3oc4c5ccccc5c5ccccc5c4c3cc2N(c2ccccc2)c2cccc3c2oc2ccccc23)cc1 ; c1ccc(-c2cc3oc4c5ccccc5c5ccccc5c4c3cc2N(c2ccccc2)c2ccc3c(c2)oc2ccccc23)cc1 ; c1ccc(-c2cc3oc4c5ccccc5c5ccccc5c4c3cc2N(c2ccccc2)c2ccc(-c3cccc4c3oc3ccccc34)cc2)cc1]

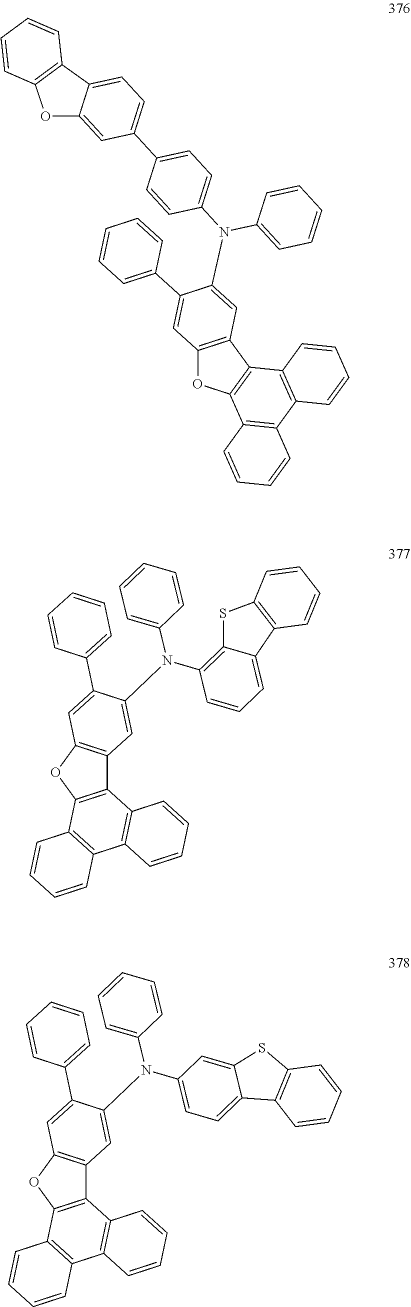 [SMILES: c1ccc(-c2cc3oc4c5ccccc5c5ccccc5c4c3cc2N(c2ccccc2)c2ccc(-c3ccc4c(c3)oc3ccccc34)cc2)cc1 ; c1ccc(-c2cc3oc4c5ccccc5c5ccccc5c4c3cc2N(c2ccccc2)c2cccc3c2sc2ccccc23)cc1 ; c1ccc(-c2cc3oc4c5ccccc5c5ccccc5c4c3cc2N(c2ccccc2)c2ccc3c(c2)sc2ccccc23)cc1]

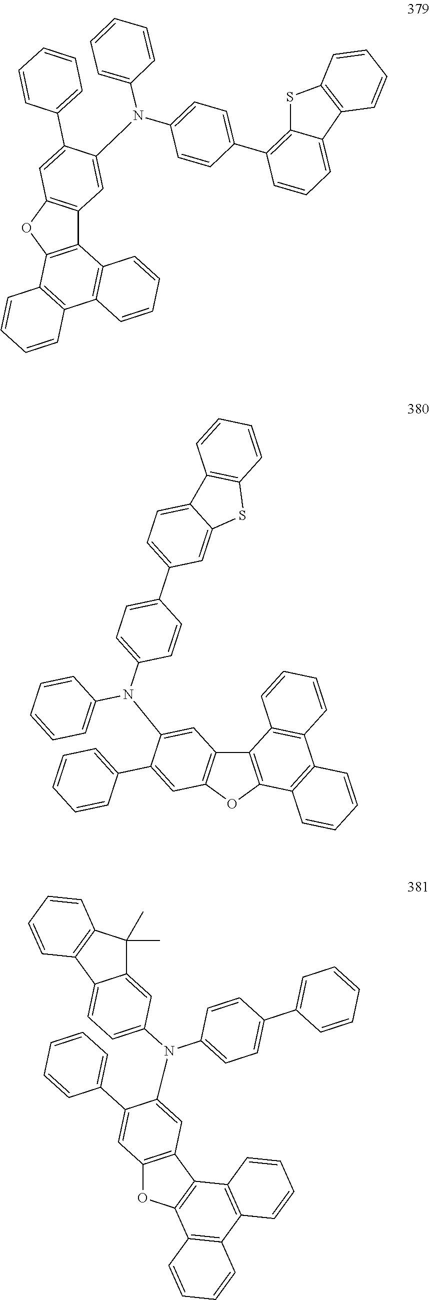 [SMILES: c1ccc(-c2cc3oc4c5ccccc5c5ccccc5c4c3cc2N(c2ccccc2)c2ccc(-c3cccc4c3sc3ccccc34)cc2)cc1 ; c1ccc(-c2cc3oc4c5ccccc5c5ccccc5c4c3cc2N(c2ccccc2)c2ccc(-c3ccc4c(c3)sc3ccccc34)cc2)cc1 ; CC1(C)c2ccccc2-c2ccc(N(c3ccc(-c4ccccc4)cc3)c3cc4c(cc3-c3ccccc3)oc3c5ccccc5c5ccccc5c43)cc21]

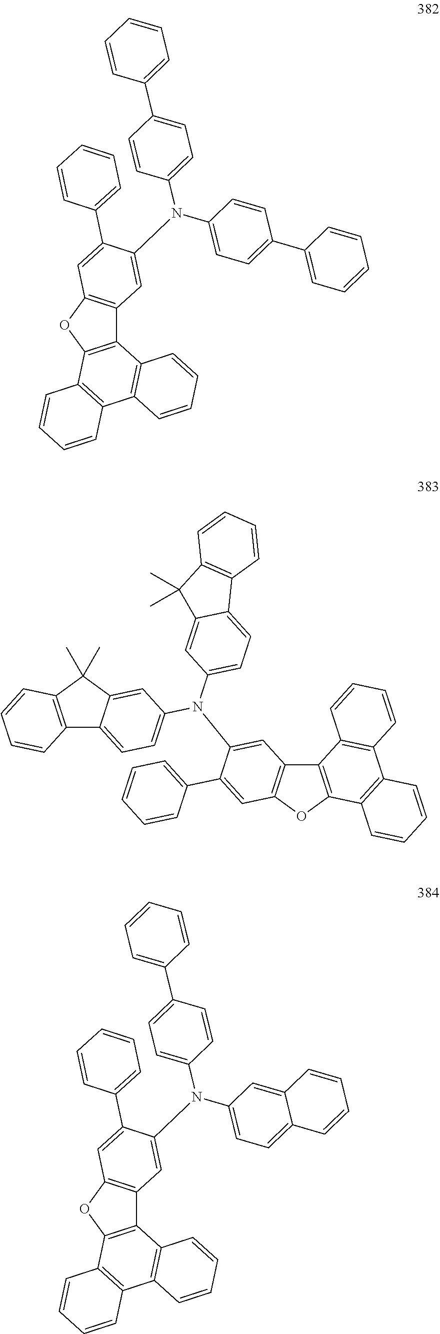 [SMILES: c1ccc(-c2ccc(N(c3ccc(-c4ccccc4)cc3)c3cc4c(cc3-c3ccccc3)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccccc3)oc3c5ccccc5c5ccccc5c43)cc21 ; c1ccc(-c2ccc(N(c3ccc4ccccc4c3)c3cc4c(cc3-c3ccccc3)oc3c5ccccc5c5ccccc5c43)cc2)cc1]

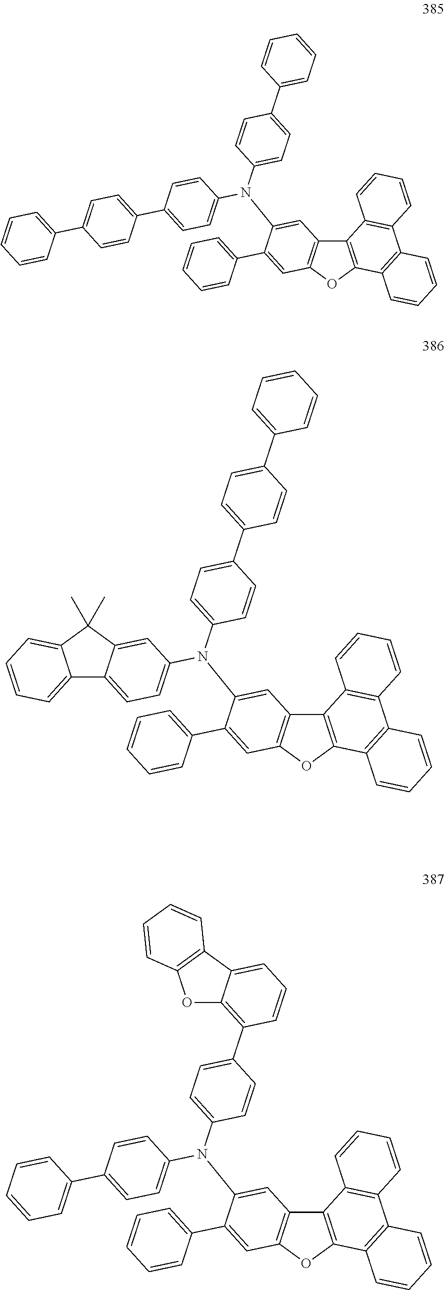 [SMILES: c1ccc(-c2ccc(-c3ccc(N(c4ccc(-c5ccccc5)cc4)c4cc5c(cc4-c4ccccc4)oc4c6ccccc6c6ccccc6c54)cc3)cc2)cc1 ; CC1(C)c2ccccc2-c2ccc(N(c3ccc(-c4ccc(-c5ccccc5)cc4)cc3)c3cc4c(cc3-c3ccccc3)oc3c5ccccc5c5ccccc5c43)cc21 ; c1ccc(-c2ccc(N(c3ccc(-c4cccc5c4oc4ccccc45)cc3)c3cc4c(cc3-c3ccccc3)oc3c5ccccc5c5ccccc5c43)cc2)cc1]

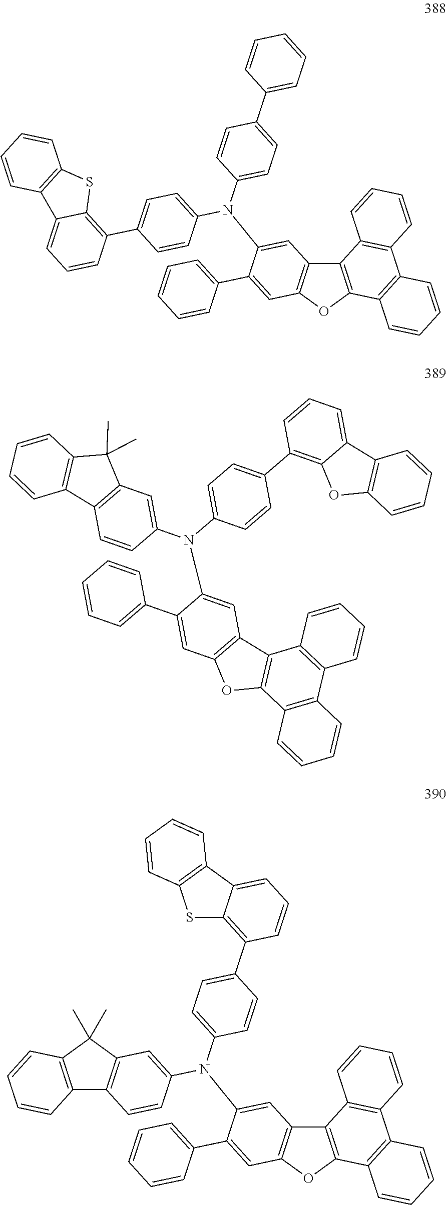 [SMILES: c1ccc(-c2ccc(N(c3ccc(-c4cccc5c4sc4ccccc45)cc3)c3cc4c(cc3-c3ccccc3)oc3c5ccccc5c5ccccc5c43)cc2)cc1 ; CC1(C)c2ccccc2-c2ccc(N(c3ccc(-c4cccc5c4oc4ccccc45)cc3)c3cc4c(cc3-c3ccccc3)oc3c5ccccc5c5ccccc5c43)cc21 ; CC1(C)c2ccccc2-c2ccc(N(c3ccc(-c4cccc5c4sc4ccccc45)cc3)c3cc4c(cc3-c3ccccc3)oc3c5ccccc5c5ccccc5c43)cc21]

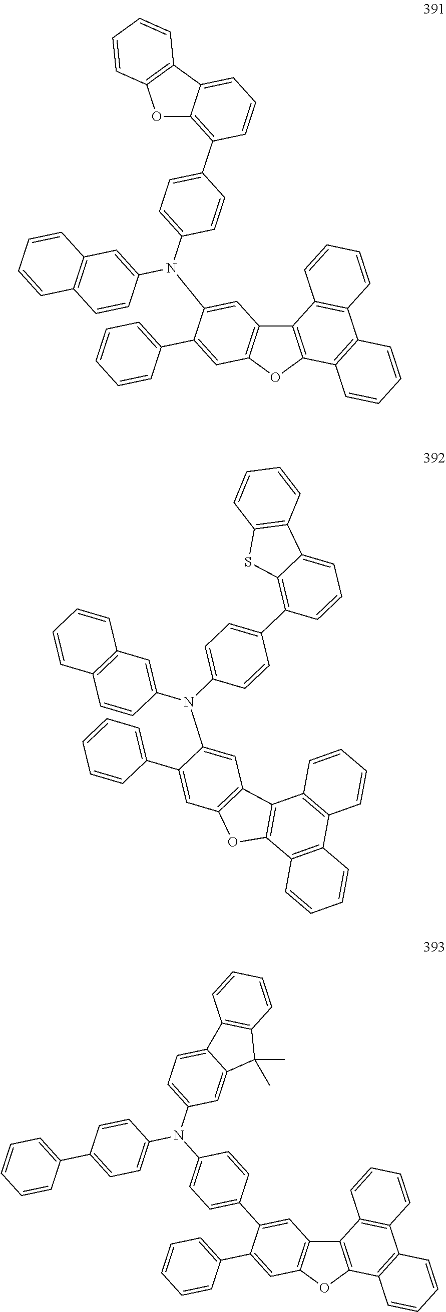 [SMILES: c1ccc(-c2cc3oc4c5ccccc5c5ccccc5c4c3cc2N(c2ccc(-c3cccc4c3oc3ccccc34)cc2)c2ccc3ccccc3c2)cc1 ; c1ccc(-c2cc3oc4c5ccccc5c5ccccc5c4c3cc2N(c2ccc(-c3cccc4c3sc3ccccc34)cc2)c2ccc3ccccc3c2)cc1 ; CC1(C)c2ccccc2-c2ccc(N(c3ccc(-c4ccccc4)cc3)c3ccc(-c4cc5c(cc4-c4ccccc4)oc4c6ccccc6c6ccccc6c54)cc3)cc21]

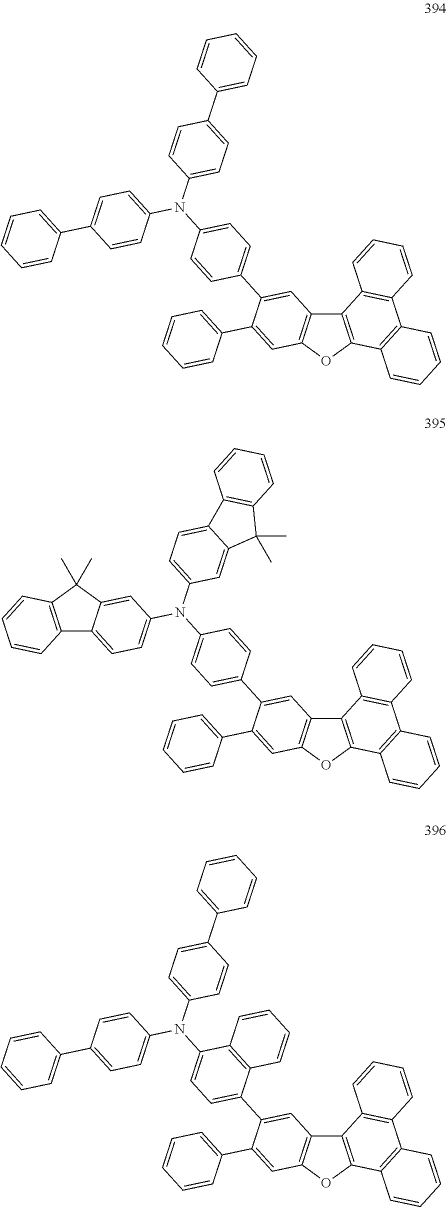 [SMILES: c1ccc(-c2ccc(N(c3ccc(-c4ccccc4)cc3)c3ccc(-c4cc5c(cc4-c4ccccc4)oc4c6ccccc6c6ccccc6c54)cc3)cc2)cc1 ; CC1(C)c2ccccc2-c2ccc(N(c3ccc(-c4cc5c(cc4-c4ccccc4)oc4c6ccccc6c6ccccc6c54)cc3)c3ccc4c(c3)C(C)(C)c3ccccc3-4)cc21 ; c1ccc(-c2ccc(N(c3ccc(-c4ccccc4)cc3)c3ccc(-c4cc5c(cc4-c4ccccc4)oc4c6ccccc6c6ccccc6c54)c4ccccc34)cc2)cc1]

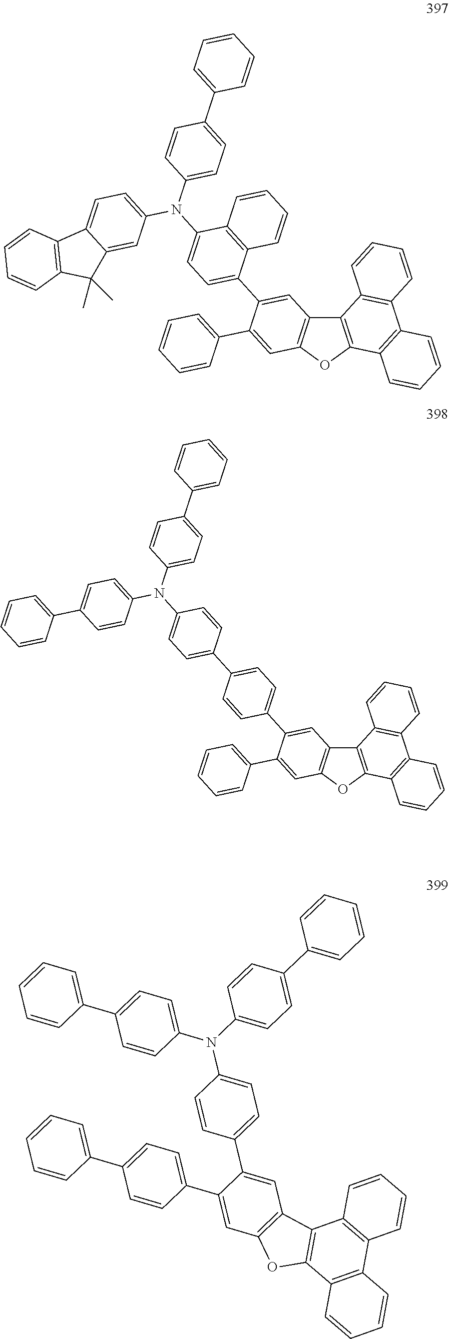 [SMILES: CC1(C)c2ccccc2-c2ccc(N(c3ccc(-c4ccccc4)cc3)c3ccc(-c4cc5c(cc4-c4ccccc4)oc4c6ccccc6c6ccccc6c54)c4ccccc34)cc21 ; c1ccc(-c2ccc(N(c3ccc(-c4ccccc4)cc3)c3ccc(-c4ccc(-c5cc6c(cc5-c5ccccc5)oc5c7ccccc7c7ccccc7c65)cc4)cc3)cc2)cc1 ; c1ccc(-c2ccc(-c3cc4oc5c6ccccc6c6ccccc6c5c4cc3-c3ccc(N(c4ccc(-c5ccccc5)cc4)c4ccc(-c5ccccc5)cc4)cc3)cc2)cc1]

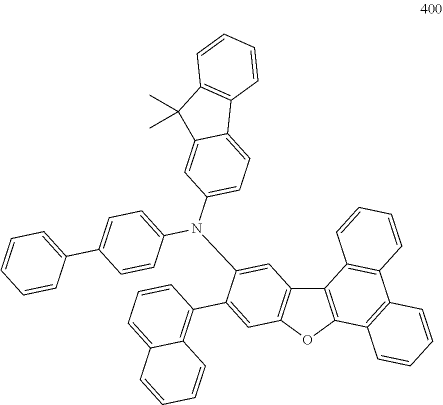 [SMILES: CC1(C)c2ccccc2-c2ccc(N(c3ccc(-c4ccccc4)cc3)c3cc4c(cc3-c3cccc5ccccc35)oc3c5ccccc5c5ccccc5c43)cc21]

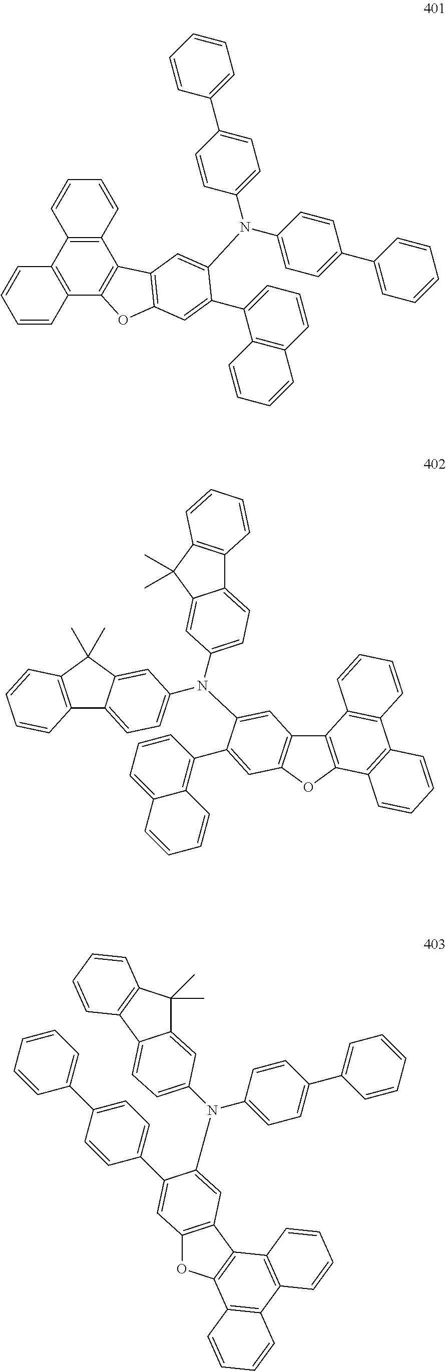 [SMILES: c1ccc(-c2ccc(N(c3ccc(-c4ccccc4)cc3)c3cc4c(cc3-c3cccc5ccccc35)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3cccc5ccccc35)oc3c5ccccc5c5ccccc5c43)cc21 ; CC1(C)c2ccccc2-c2ccc(N(c3ccc(-c4ccccc4)cc3)c3cc4c(cc3-c3ccc(-c5ccccc5)cc3)oc3c5ccccc5c5ccccc5c43)cc21]

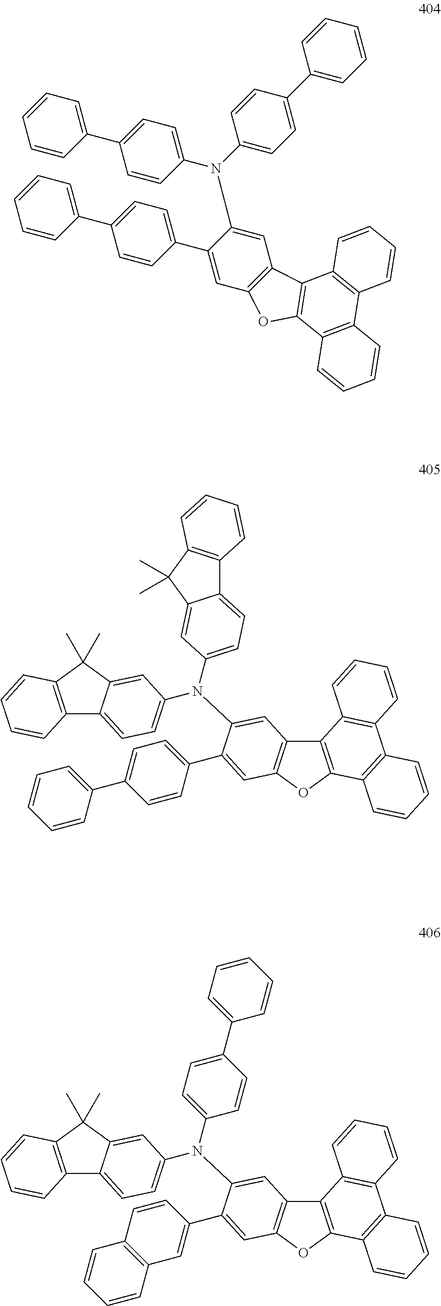 [SMILES: c1ccc(-c2ccc(-c3cc4oc5c6ccccc6c6ccccc6c5c4cc3N(c3ccc(-c4ccccc4)cc3)c3ccc(-c4ccccc4)cc3)cc2)cc1 ; CC1(C)c2ccccc2-c2ccc(N(c3ccc4c(c3)C(C)(C)c3ccccc3-4)c3cc4c(cc3-c3ccc(-c5ccccc5)cc3)oc3c5ccccc5c5ccccc5c43)cc21 ; CC1(C)c2ccccc2-c2ccc(N(c3ccc(-c4ccccc4)cc3)c3cc4c(cc3-c3ccc5ccccc5c3)oc3c5ccccc5c5ccccc5c43)cc21]

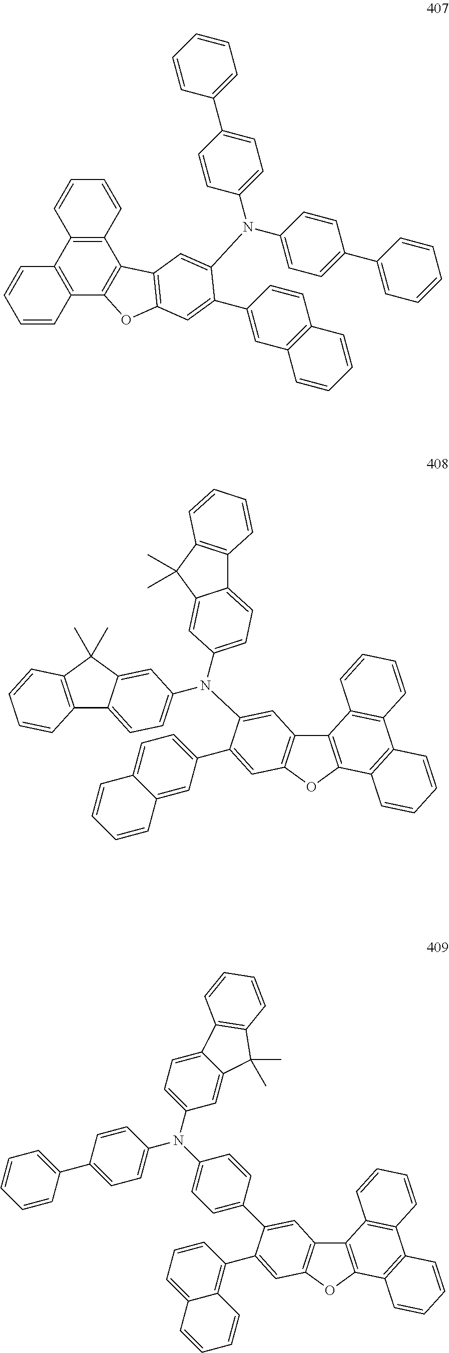 [SMILES: c1ccc(-c2ccc(N(c3ccc(-c4ccccc4)cc3)c3cc4c(cc3-c3ccc5ccccc5c3)oc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccc5ccccc5c3)oc3c5ccccc5c5ccccc5c43)cc21 ; CC1(C)c2ccccc2-c2ccc(N(c3ccc(-c4ccccc4)cc3)c3ccc(-c4cc5c(cc4-c4cccc6ccccc46)oc4c6ccccc6c6ccccc6c54)cc3)cc21]

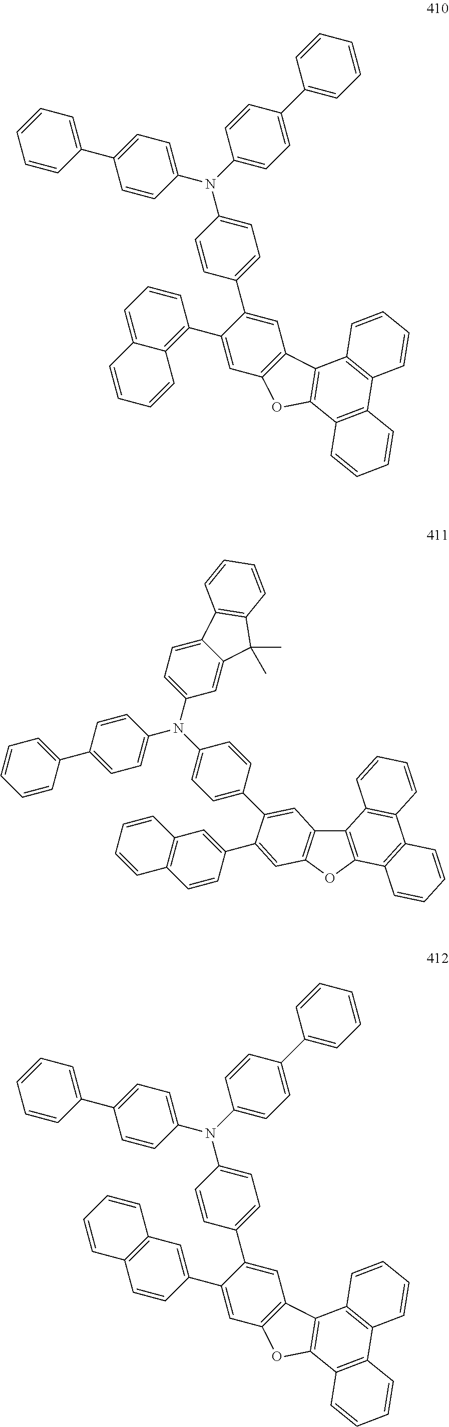 [SMILES: c1ccc(-c2ccc(N(c3ccc(-c4ccccc4)cc3)c3ccc(-c4cc5c(cc4-c4cccc6ccccc46)oc4c6ccccc6c6ccccc6c54)cc3)cc2)cc1 ; CC1(C)c2ccccc2-c2ccc(N(c3ccc(-c4ccccc4)cc3)c3ccc(-c4cc5c(cc4-c4ccc6ccccc6c4)oc4c6ccccc6c6ccccc6c54)cc3)cc21 ; c1ccc(-c2ccc(N(c3ccc(-c4ccccc4)cc3)c3ccc(-c4cc5c(cc4-c4ccc6ccccc6c4)oc4c6ccccc6c6ccccc6c54)cc3)cc2)cc1]

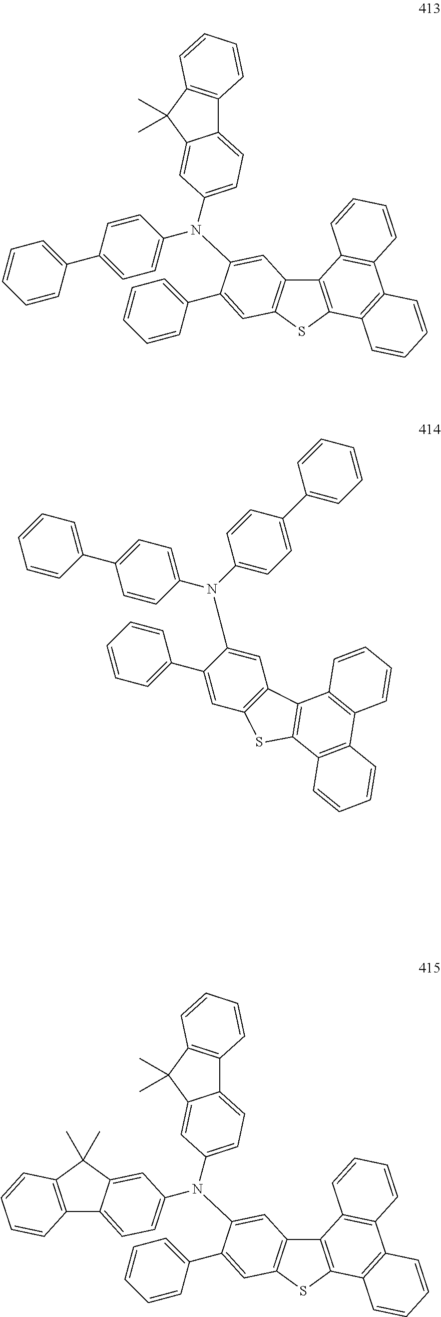 [SMILES: CC1(C)c2ccccc2-c2ccc(N(c3ccc(-c4ccccc4)cc3)c3cc4c(cc3-c3ccccc3)sc3c5ccccc5c5ccccc5c43)cc21 ; c1ccc(-c2ccc(N(c3ccc(-c4ccccc4)cc3)c3cc4c(cc3-c3ccccc3)sc3c5ccccc5c5ccccc5c43)cc2)cc1 ; CC1(C)c2ccccc2-c2ccc(N(c3ccc4c(c3)C(C)(C)c3ccccc3-4)c3cc4c(cc3-c3ccccc3)sc3c5ccccc5c5ccccc5c43)cc21]

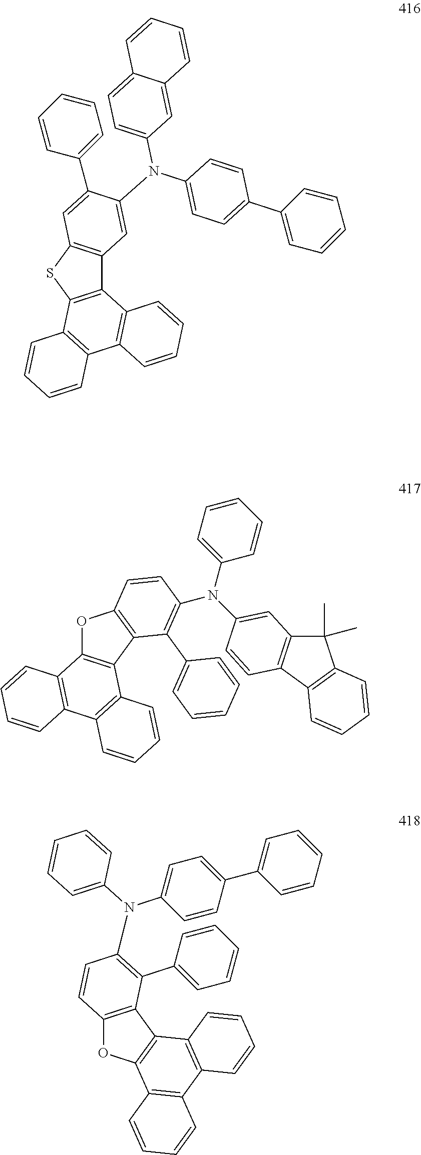 [SMILES: c1ccc(-c2ccc(N(c3ccc4ccccc4c3)c3cc4c(cc3-c3ccccc3)sc3c5ccccc5c5ccccc5c43)cc2)cc1 ; CC1(C)c2ccccc2-c2ccc(N(c3ccccc3)c3ccc4oc5c6ccccc6c6ccccc6c5c4c3-c3ccccc3)cc21 ; c1ccc(-c2ccc(N(c3ccccc3)c3ccc4oc5c6ccccc6c6ccccc6c5c4c3-c3ccccc3)cc2)cc1]

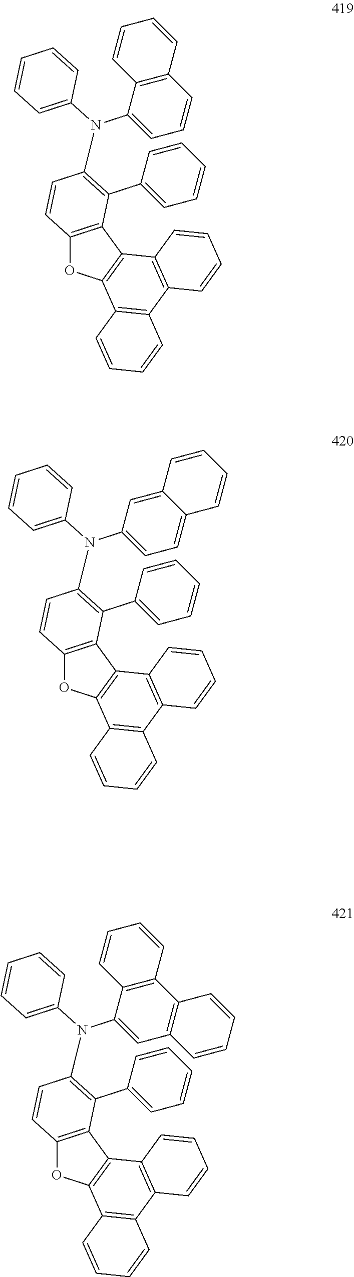 [SMILES: c1ccc(-c2c(N(c3ccccc3)c3cccc4ccccc34)ccc3oc4c5ccccc5c5ccccc5c4c23)cc1 ; c1ccc(-c2c(N(c3ccccc3)c3ccc4ccccc4c3)ccc3oc4c5ccccc5c5ccccc5c4c23)cc1 ; c1ccc(-c2c(N(c3ccccc3)c3cc4ccccc4c4ccccc34)ccc3oc4c5ccccc5c5ccccc5c4c23)cc1]

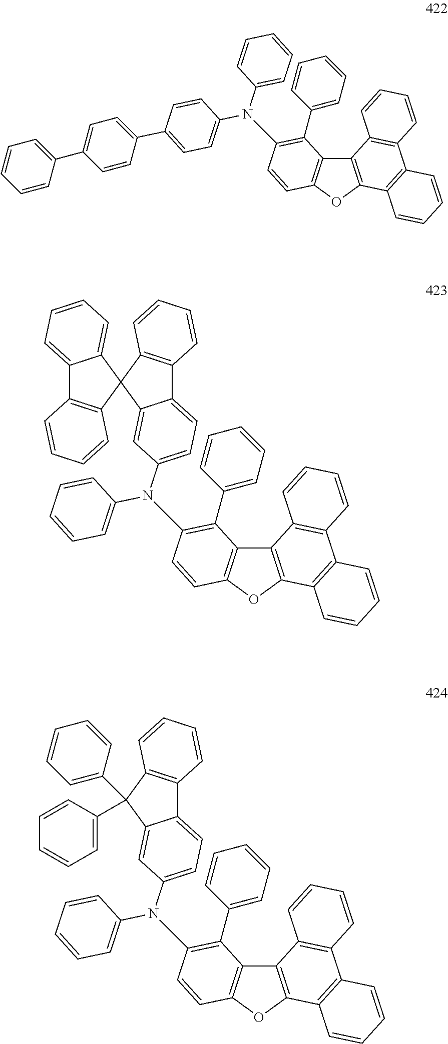 [SMILES: c1ccc(-c2ccc(-c3ccc(N(c4ccccc4)c4ccc5oc6c7ccccc7c7ccccc7c6c5c4-c4ccccc4)cc3)cc2)cc1 ; c1ccc(-c2c(N(c3ccccc3)c3ccc4c(c3)C3(c5ccccc5-c5ccccc53)c3ccccc3-4)ccc3oc4c5ccccc5c5ccccc5c4c23)cc1 ; c1ccc(-c2c(N(c3ccccc3)c3ccc4c(c3)C(c3ccccc3)(c3ccccc3)c3ccccc3-4)ccc3oc4c5ccccc5c5ccccc5c4c23)cc1]

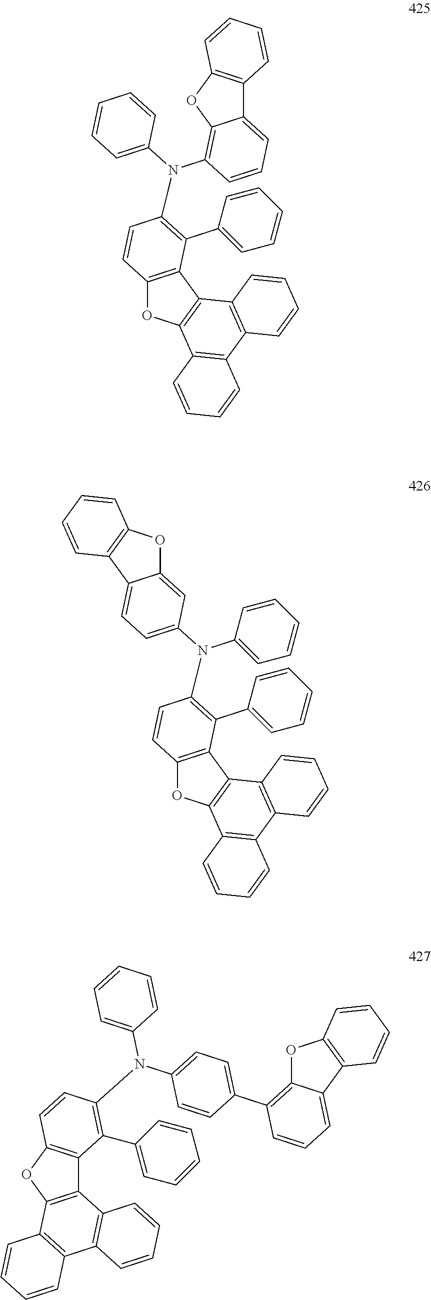 [SMILES: c1ccc(-c2c(N(c3ccccc3)c3cccc4c3oc3ccccc34)ccc3oc4c5ccccc5c5ccccc5c4c23)cc1 ; c1ccc(-c2c(N(c3ccccc3)c3ccc4c(c3)oc3ccccc34)ccc3oc4c5ccccc5c5ccccc5c4c23)cc1 ; c1ccc(-c2c(N(c3ccccc3)c3ccc(-c4cccc5c4oc4ccccc45)cc3)ccc3oc4c5ccccc5c5ccccc5c4c23)cc1]

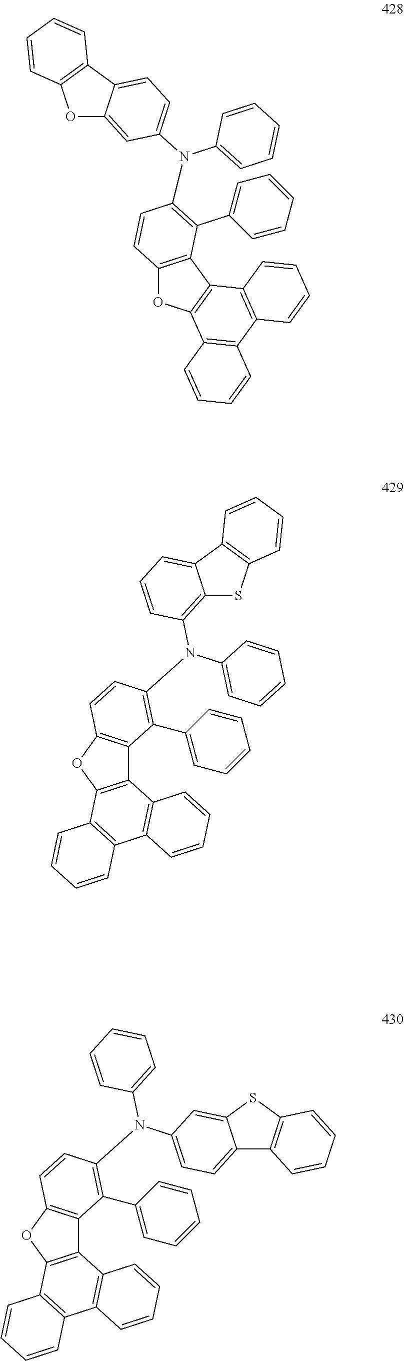 [SMILES: c1ccc(-c2c(N(c3ccccc3)c3ccc4c(c3)oc3ccccc34)ccc3oc4c5ccccc5c5ccccc5c4c23)cc1 ; c1ccc(-c2c(N(c3ccccc3)c3cccc4c3sc3ccccc34)ccc3oc4c5ccccc5c5ccccc5c4c23)cc1 ; c1ccc(-c2c(N(c3ccccc3)c3ccc4c(c3)sc3ccccc34)ccc3oc4c5ccccc5c5ccccc5c4c23)cc1]

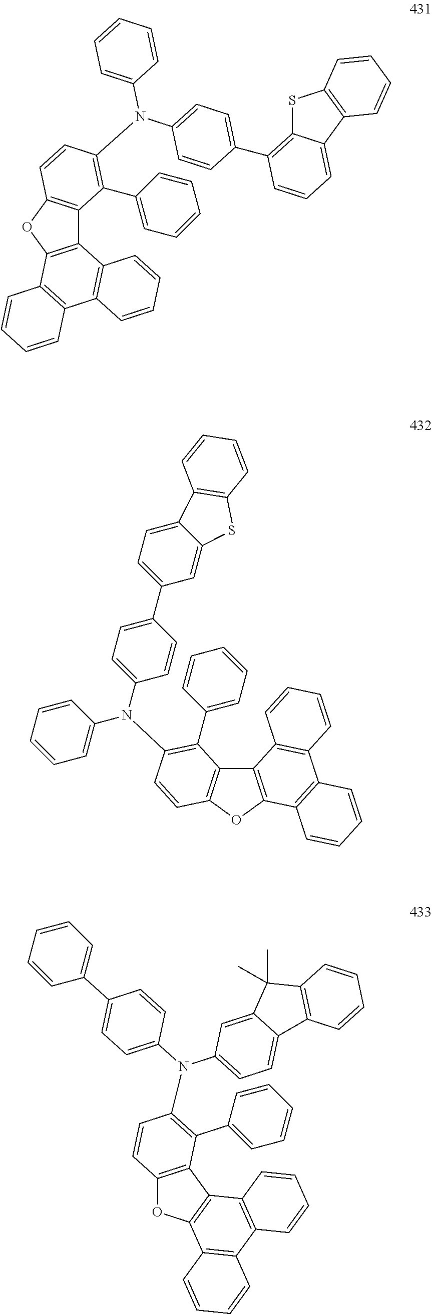 [SMILES: c1ccc(-c2c(N(c3ccccc3)c3ccc(-c4cccc5c4sc4ccccc45)cc3)ccc3oc4c5ccccc5c5ccccc5c4c23)cc1 ; c1ccc(-c2c(N(c3ccccc3)c3ccc(-c4ccc5c(c4)sc4ccccc45)cc3)ccc3oc4c5ccccc5c5ccccc5c4c23)cc1 ; CC1(C)c2ccccc2-c2ccc(N(c3ccc(-c4ccccc4)cc3)c3ccc4oc5c6ccccc6c6ccccc6c5c4c3-c3ccccc3)cc21]

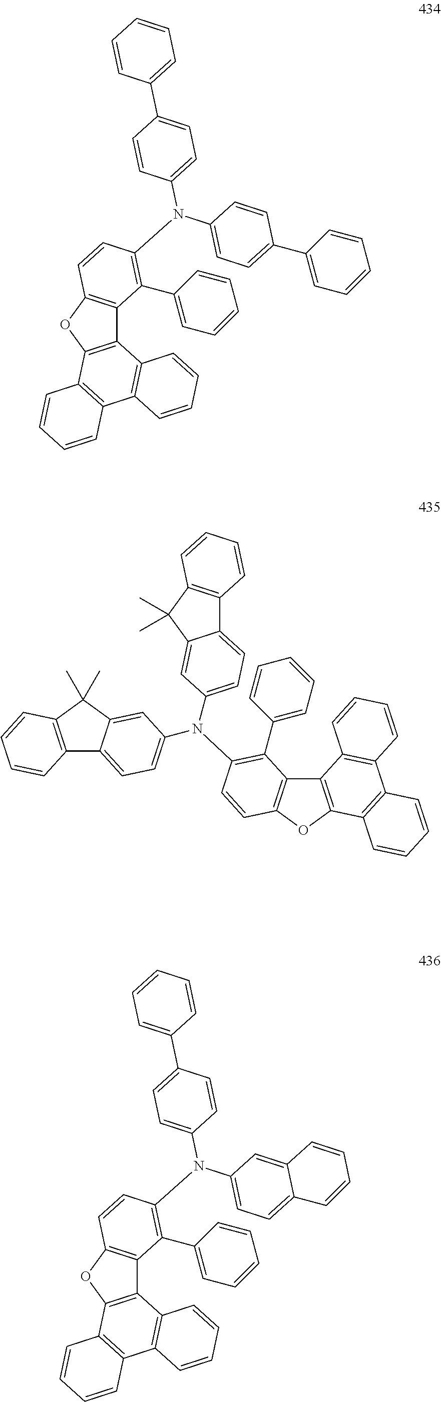 [SMILES: c1ccc(-c2ccc(N(c3ccc(-c4ccccc4)cc3)c3ccc4oc5c6ccccc6c6ccccc6c5c4c3-c3ccccc3)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccccc3)cc21 ; c1ccc(-c2ccc(N(c3ccc4ccccc4c3)c3ccc4oc5c6ccccc6c6ccccc6c5c4c3-c3ccccc3)cc2)cc1]

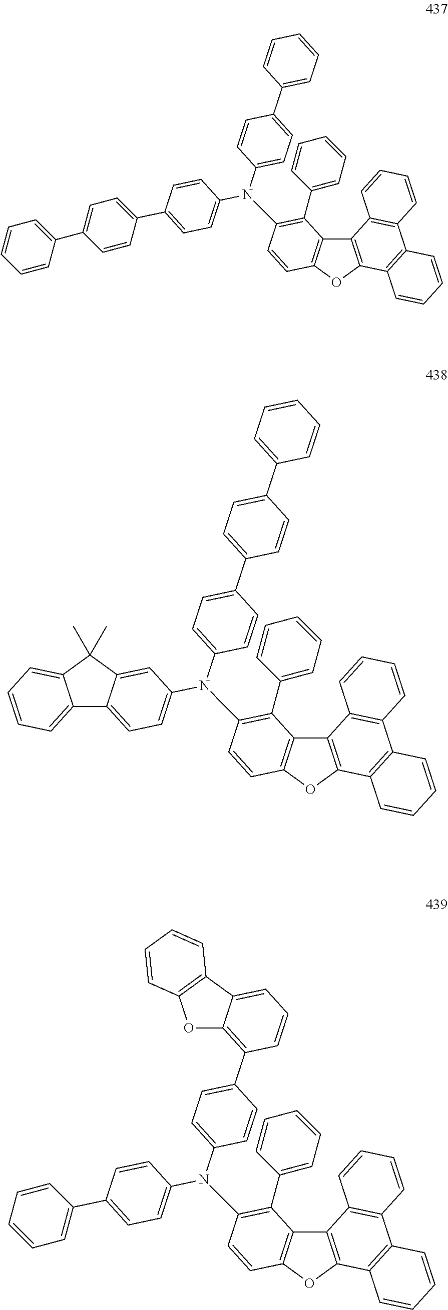 [SMILES: c1ccc(-c2ccc(-c3ccc(N(c4ccc(-c5ccccc5)cc4)c4ccc5oc6c7ccccc7c7ccccc7c6c5c4-c4ccccc4)cc3)cc2)cc1 ; CC1(C)c2ccccc2-c2ccc(N(c3ccc(-c4ccc(-c5ccccc5)cc4)cc3)c3ccc4oc5c6ccccc6c6ccccc6c5c4c3-c3ccccc3)cc21 ; c1ccc(-c2ccc(N(c3ccc(-c4cccc5c4oc4ccccc45)cc3)c3ccc4oc5c6ccccc6c6ccccc6c5c4c3-c3ccccc3)cc2)cc1]

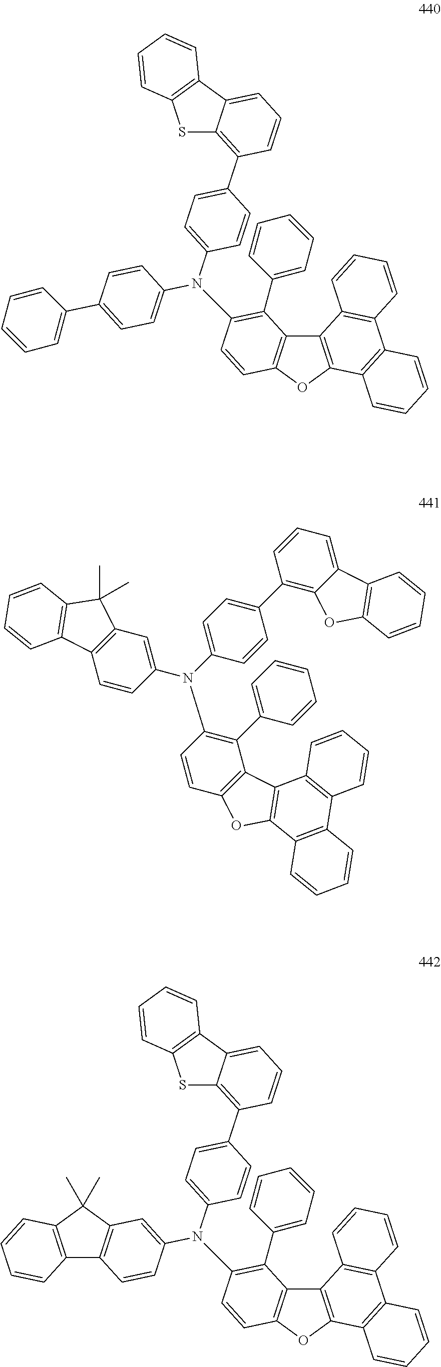 [SMILES: c1ccc(-c2ccc(N(c3ccc(-c4cccc5c4sc4ccccc45)cc3)c3ccc4oc5c6ccccc6c6ccccc6c5c4c3-c3ccccc3)cc2)cc1 ; CC1(C)c2ccccc2-c2ccc(N(c3ccc(-c4cccc5c4oc4ccccc45)cc3)c3ccc4oc5c6ccccc6c6ccccc6c5c4c3-c3ccccc3)cc21 ; CC1(C)c2ccccc2-c2ccc(N(c3ccc(-c4cccc5c4sc4ccccc45)cc3)c3ccc4oc5c6ccccc6c6ccccc6c5c4c3-c3ccccc3)cc21]

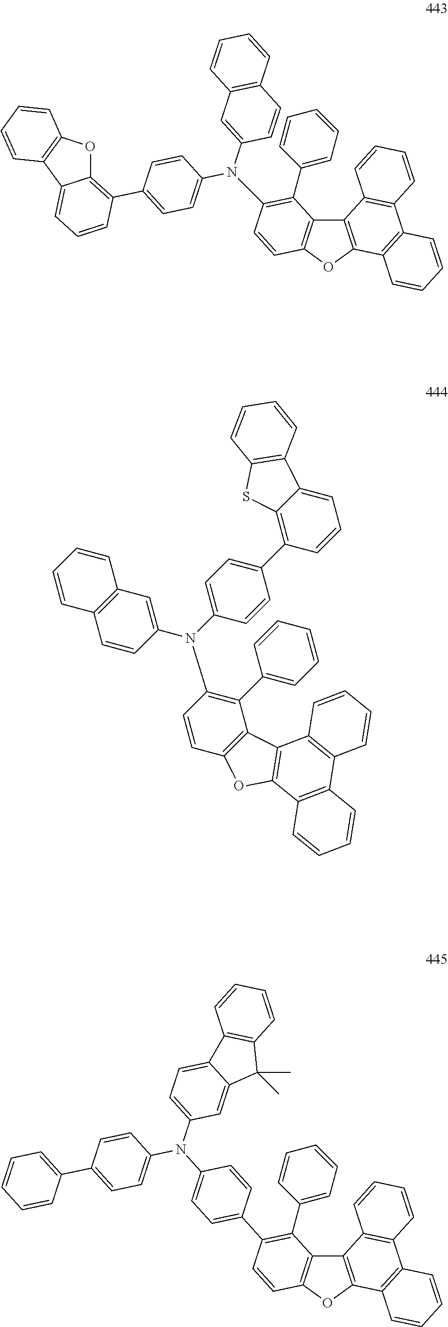 [SMILES: c1ccc(-c2c(N(c3ccc(-c4cccc5c4oc4ccccc45)cc3)c3ccc4ccccc4c3)ccc3oc4c5ccccc5c5ccccc5c4c23)cc1 ; c1ccc(-c2c(N(c3ccc(-c4cccc5c4sc4ccccc45)cc3)c3ccc4ccccc4c3)ccc3oc4c5ccccc5c5ccccc5c4c23)cc1 ; CC1(C)c2ccccc2-c2ccc(N(c3ccc(-c4ccccc4)cc3)c3ccc(-c4ccc5oc6c7ccccc7c7ccccc7c6c5c4-c4ccccc4)cc3)cc21]

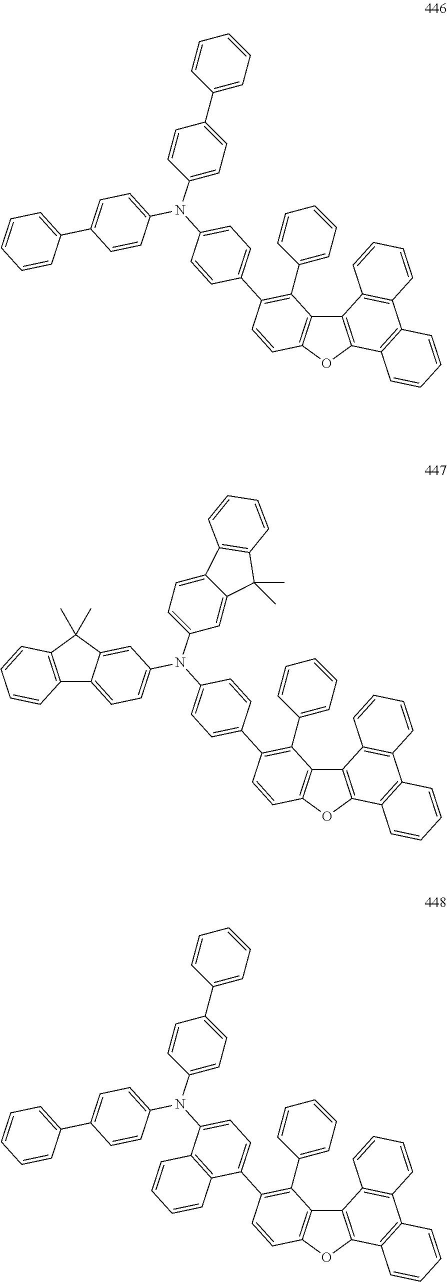 [SMILES: c1ccc(-c2ccc(N(c3ccc(-c4ccccc4)cc3)c3ccc(-c4ccc5oc6c7ccccc7c7ccccc7c6c5c4-c4ccccc4)cc3)cc2)cc1 ; CC1(C)c2ccccc2-c2ccc(N(c3ccc(-c4ccc5oc6c7ccccc7c7ccccc7c6c5c4-c4ccccc4)cc3)c3ccc4c(c3)C(C)(C)c3ccccc3-4)cc21 ; c1ccc(-c2ccc(N(c3ccc(-c4ccccc4)cc3)c3ccc(-c4ccc5oc6c7ccccc7c7ccccc7c6c5c4-c4ccccc4)c4ccccc34)cc2)cc1]

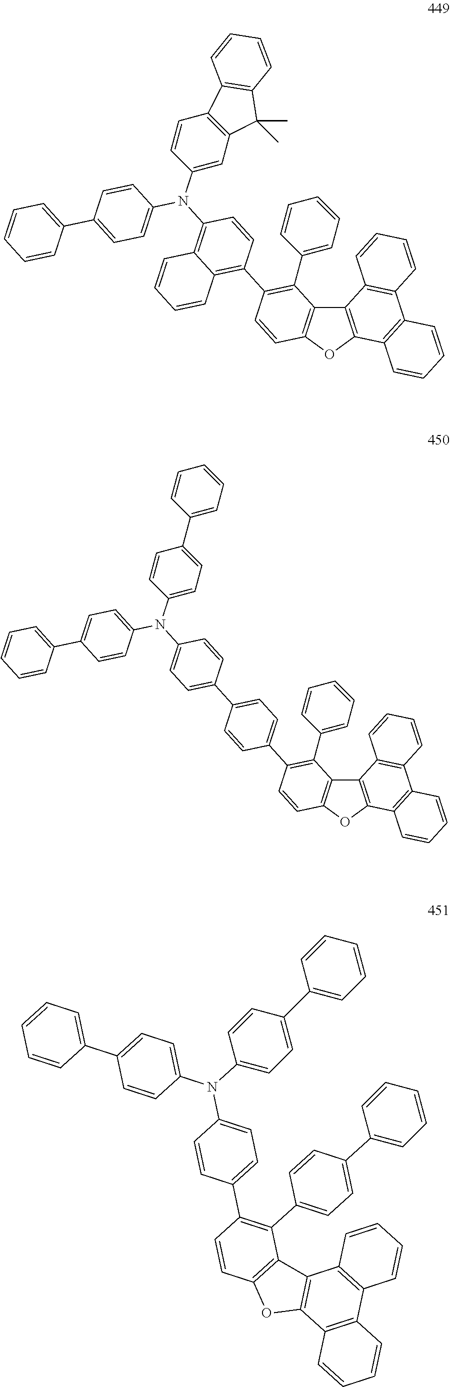 [SMILES: CC1(C)c2ccccc2-c2ccc(N(c3ccc(-c4ccccc4)cc3)c3ccc(-c4ccc5oc6c7ccccc7c7ccccc7c6c5c4-c4ccccc4)c4ccccc34)cc21 ; c1ccc(-c2ccc(N(c3ccc(-c4ccccc4)cc3)c3ccc(-c4ccc(-c5ccc6oc7c8ccccc8c8ccccc8c7c6c5-c5ccccc5)cc4)cc3)cc2)cc1 ; c1ccc(-c2ccc(-c3c(-c4ccc(N(c5ccc(-c6ccccc6)cc5)c5ccc(-c6ccccc6)cc5)cc4)ccc4oc5c6ccccc6c6ccccc6c5c34)cc2)cc1]

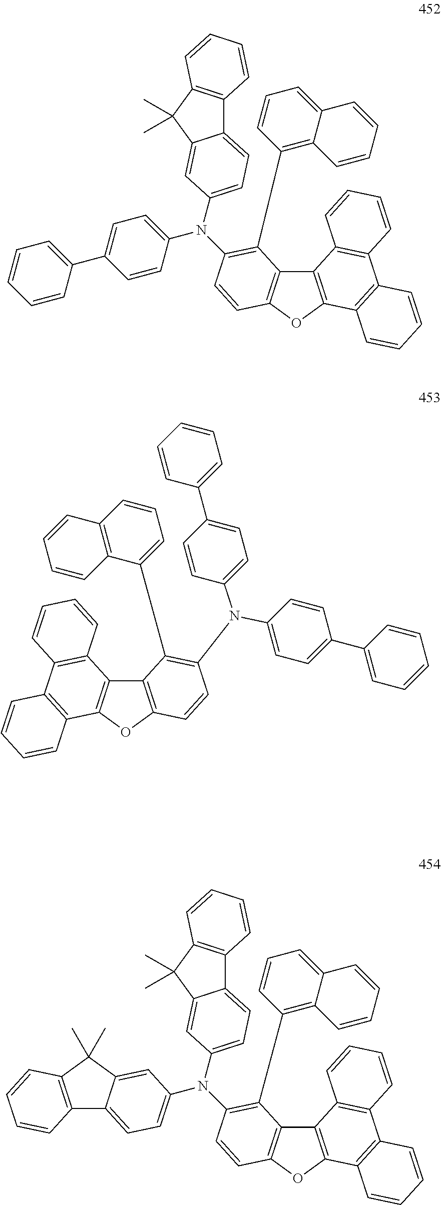 [SMILES: CC1(C)c2ccccc2-c2ccc(N(c3ccc(-c4ccccc4)cc3)c3ccc4oc5c6ccccc6c6ccccc6c5c4c3-c3cccc4ccccc34)cc21 ; c1ccc(-c2ccc(N(c3ccc(-c4ccccc4)cc3)c3ccc4oc5c6ccccc6c6ccccc6c5c4c3-c3cccc4ccccc34)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3cccc4ccccc34)cc21]

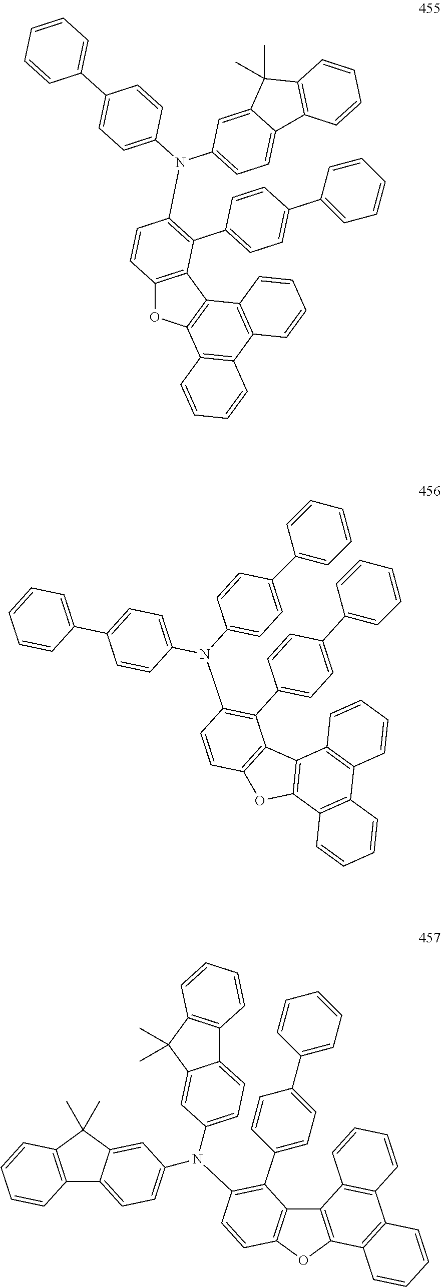 [SMILES: CC1(C)c2ccccc2-c2ccc(N(c3ccc(-c4ccccc4)cc3)c3ccc4oc5c6ccccc6c6ccccc6c5c4c3-c3ccc(-c4ccccc4)cc3)cc21 ; c1ccc(-c2ccc(-c3c(N(c4ccc(-c5ccccc5)cc4)c4ccc(-c5ccccc5)cc4)ccc4oc5c6ccccc6c6ccccc6c5c34)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccc(-c4ccccc4)cc3)cc21]

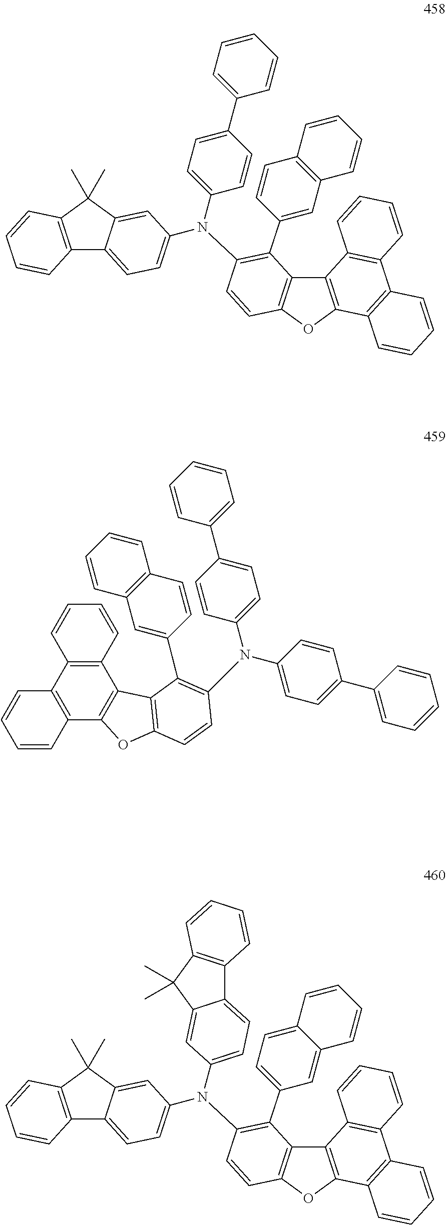 [SMILES: CC1(C)c2ccccc2-c2ccc(N(c3ccc(-c4ccccc4)cc3)c3ccc4oc5c6ccccc6c6ccccc6c5c4c3-c3ccc4ccccc4c3)cc21 ; c1ccc(-c2ccc(N(c3ccc(-c4ccccc4)cc3)c3ccc4oc5c6ccccc6c6ccccc6c5c4c3-c3ccc4ccccc4c3)cc2)cc1 ; CC1(C)c2ccccc2-c2ccc(N(c3ccc4c(c3)C(C)(C)c3ccccc3-4)c3ccc4oc5c6ccccc6c6ccccc6c5c4c3-c3ccc4ccccc4c3)cc21]

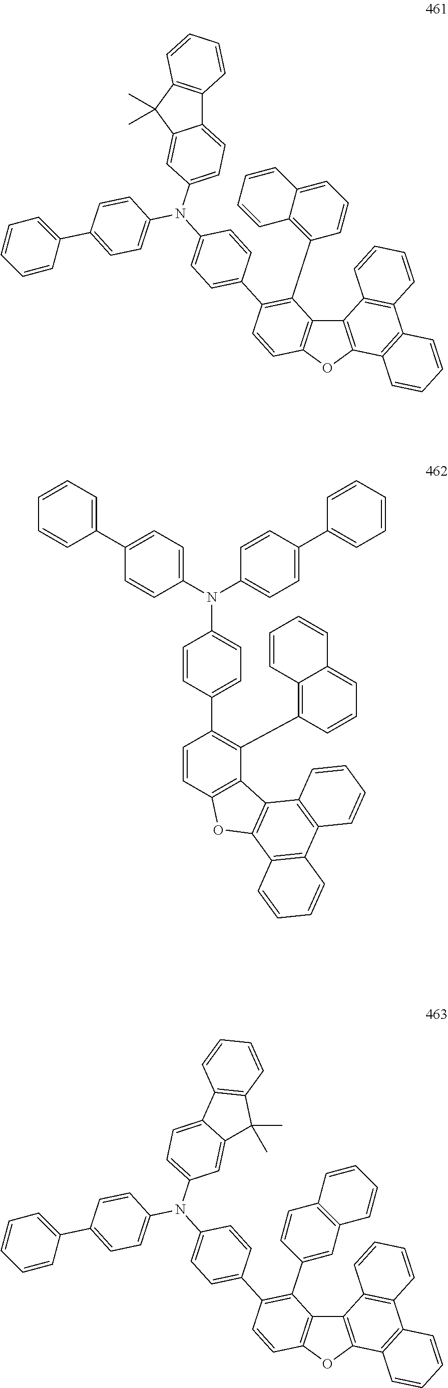 [SMILES: CC1(C)c2ccccc2-c2ccc(N(c3ccc(-c4ccccc4)cc3)c3ccc(-c4ccc5oc6c7ccccc7c7ccccc7c6c5c4-c4cccc5ccccc45)cc3)cc21 ; c1ccc(-c2ccc(N(c3ccc(-c4ccccc4)cc3)c3ccc(-c4ccc5oc6c7ccccc7c7ccccc7c6c5c4-c4cccc5ccccc45)cc3)cc2)cc1 ; CC1(C)c2ccccc2-c2ccc(N(c3ccc(-c4ccccc4)cc3)c3ccc(-c4ccc5oc6c7ccccc7c7ccccc7c6c5c4-c4ccc5ccccc5c4)cc3)cc21]

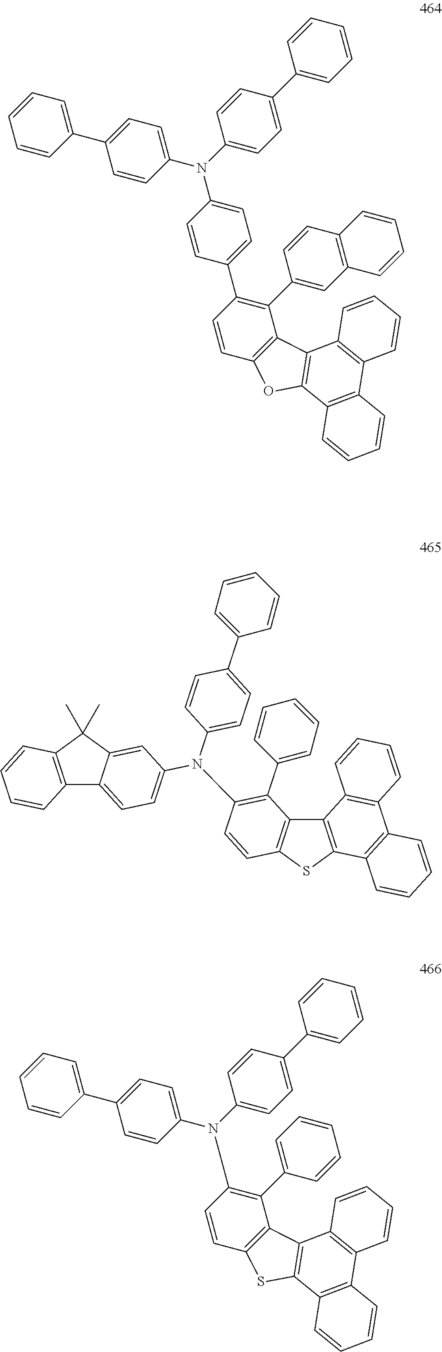 [SMILES: c1ccc(-c2ccc(N(c3ccc(-c4ccccc4)cc3)c3ccc(-c4ccc5oc6c7ccccc7c7ccccc7c6c5c4-c4ccc5ccccc5c4)cc3)cc2)cc1 ; CC1(C)c2ccccc2-c2ccc(N(c3ccc(-c4ccccc4)cc3)c3ccc4sc5c6ccccc6c6ccccc6c5c4c3-c3ccccc3)cc21 ; c1ccc(-c2ccc(N(c3ccc(-c4ccccc4)cc3)c3ccc4sc5c6ccccc6c6ccccc6c5c4c3-c3ccccc3)cc2)cc1]

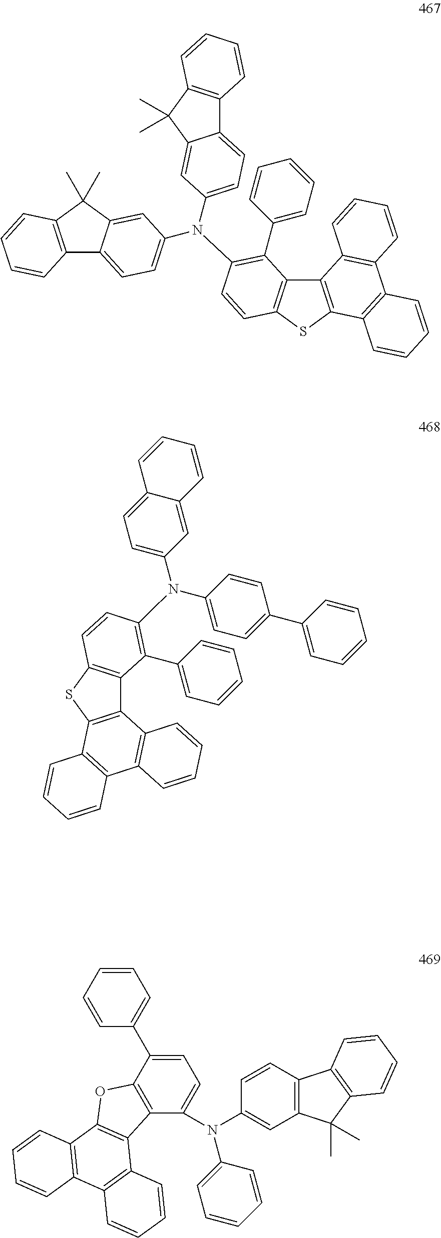 [SMILES: CC1(C)c2ccccc2-c2ccc(N(c3ccc4c(c3)C(C)(C)c3ccccc3-4)c3ccc4sc5c6ccccc6c6ccccc6c5c4c3-c3ccccc3)cc21 ; c1ccc(-c2ccc(N(c3ccc4ccccc4c3)c3ccc4sc5c6ccccc6c6ccccc6c5c4c3-c3ccccc3)cc2)cc1 ; CC1(C)c2ccccc2-c2ccc(N(c3ccccc3)c3ccc(-c4ccccc4)c4oc5c6ccccc6c6ccccc6c5c34)cc21]

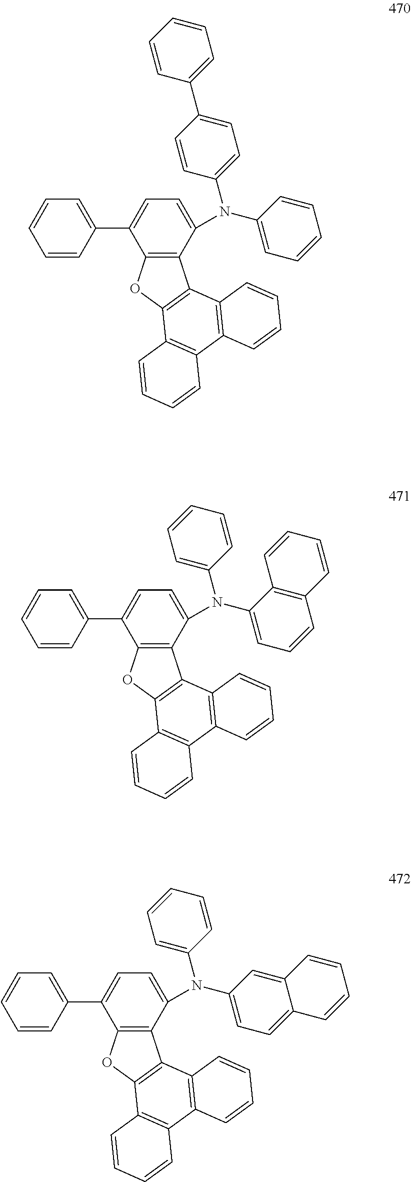 [SMILES: c1ccc(-c2ccc(N(c3ccccc3)c3ccc(-c4ccccc4)c4oc5c6ccccc6c6ccccc6c5c34)cc2)cc1 ; c1ccc(-c2ccc(N(c3ccccc3)c3cccc4ccccc34)c3c2oc2c4ccccc4c4ccccc4c23)cc1 ; c1ccc(-c2ccc(N(c3ccccc3)c3ccc4ccccc4c3)c3c2oc2c4ccccc4c4ccccc4c23)cc1]

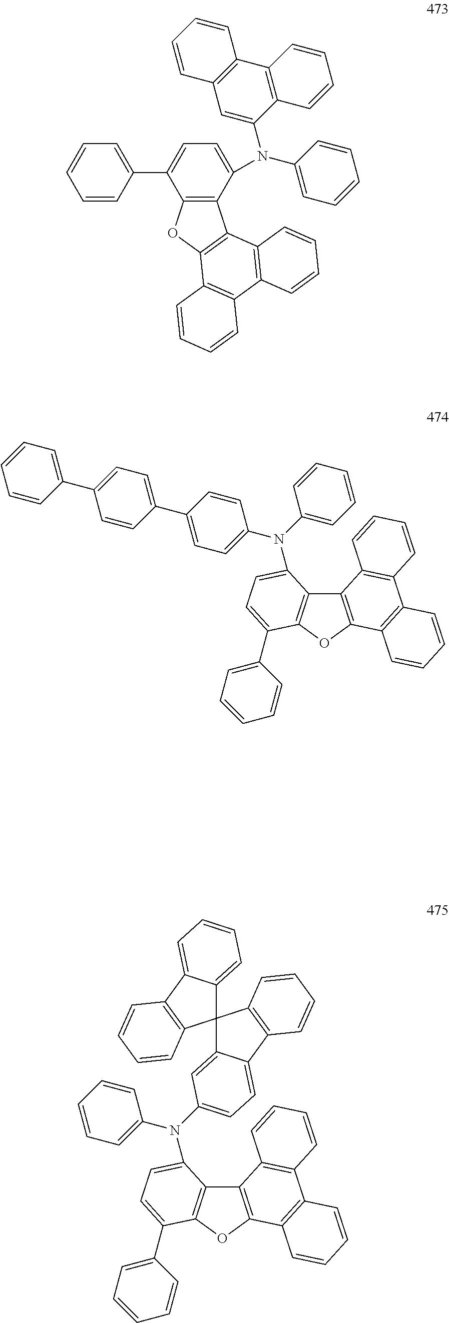 [SMILES: c1ccc(-c2ccc(N(c3ccccc3)c3cc4ccccc4c4ccccc34)c3c2oc2c4ccccc4c4ccccc4c23)cc1 ; c1ccc(-c2ccc(-c3ccc(N(c4ccccc4)c4ccc(-c5ccccc5)c5oc6c7ccccc7c7ccccc7c6c45)cc3)cc2)cc1 ; c1ccc(-c2ccc(N(c3ccccc3)c3ccc4c(c3)C3(c5ccccc5-c5ccccc53)c3ccccc3-4)c3c2oc2c4ccccc4c4ccccc4c23)cc1]

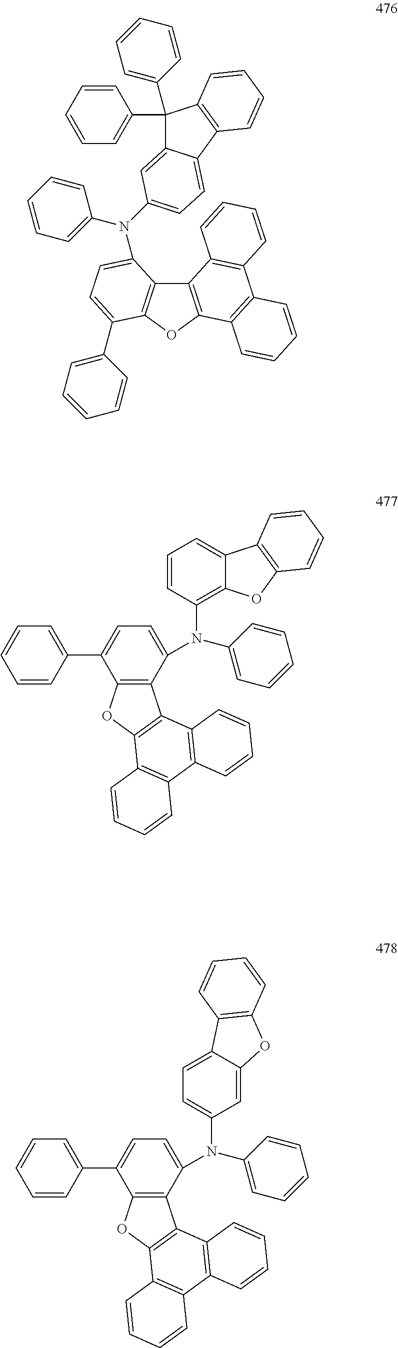 [SMILES: c1ccc(-c2ccc(N(c3ccccc3)c3ccc4c(c3)C(c3ccccc3)(c3ccccc3)c3ccccc3-4)c3c2oc2c4ccccc4c4ccccc4c23)cc1 ; c1ccc(-c2ccc(N(c3ccccc3)c3cccc4c3oc3ccccc34)c3c2oc2c4ccccc4c4ccccc4c23)cc1 ; c1ccc(-c2ccc(N(c3ccccc3)c3ccc4c(c3)oc3ccccc34)c3c2oc2c4ccccc4c4ccccc4c23)cc1]

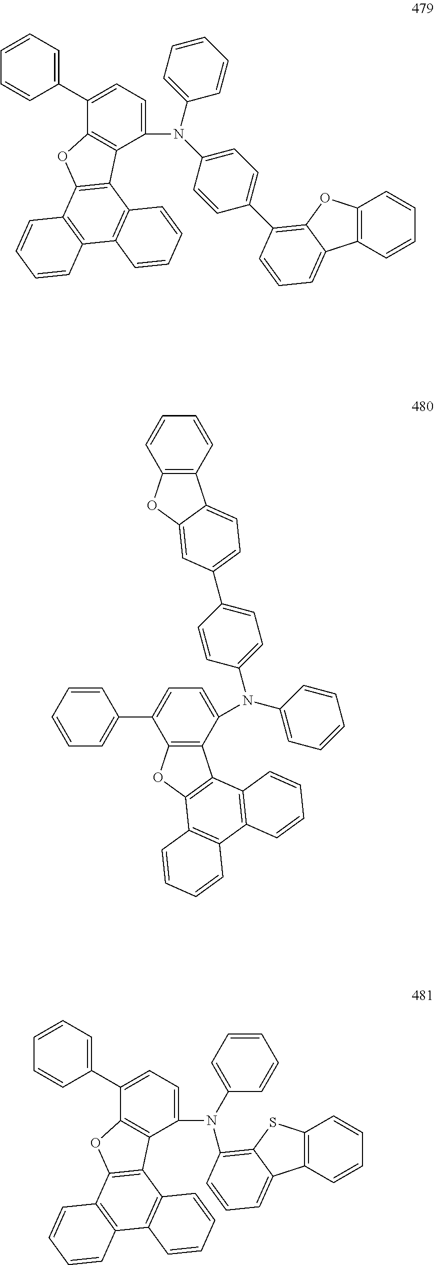 [SMILES: c1ccc(-c2ccc(N(c3ccccc3)c3ccc(-c4cccc5c4oc4ccccc45)cc3)c3c2oc2c4ccccc4c4ccccc4c23)cc1 ; c1ccc(-c2ccc(N(c3ccccc3)c3ccc(-c4ccc5c(c4)oc4ccccc45)cc3)c3c2oc2c4ccccc4c4ccccc4c23)cc1 ; c1ccc(-c2ccc(N(c3ccccc3)c3cccc4c3sc3ccccc34)c3c2oc2c4ccccc4c4ccccc4c23)cc1]

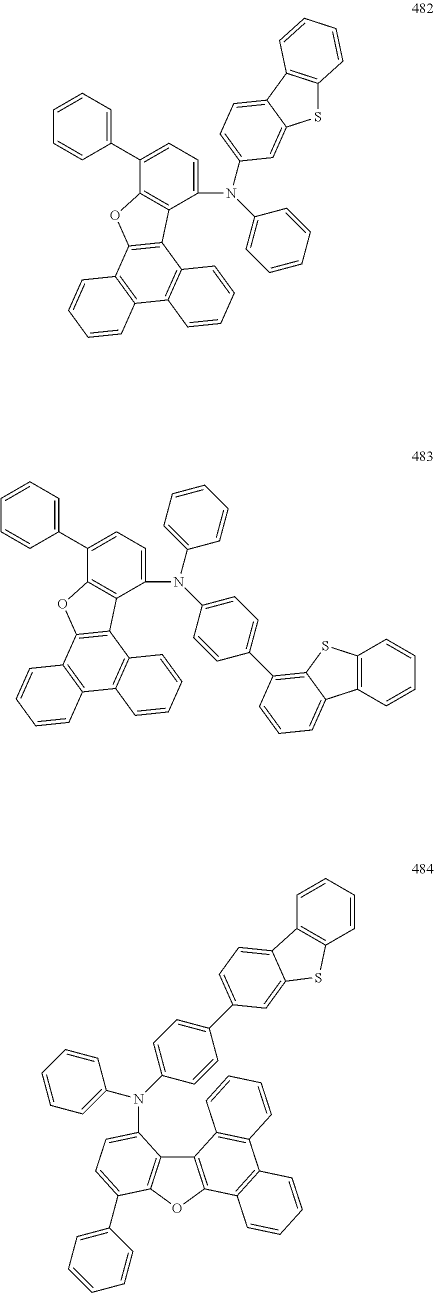 [SMILES: c1ccc(-c2ccc(N(c3ccccc3)c3ccc4c(c3)sc3ccccc34)c3c2oc2c4ccccc4c4ccccc4c23)cc1 ; c1ccc(-c2ccc(N(c3ccccc3)c3ccc(-c4cccc5c4sc4ccccc45)cc3)c3c2oc2c4ccccc4c4ccccc4c23)cc1 ; c1ccc(-c2ccc(N(c3ccccc3)c3ccc(-c4ccc5c(c4)sc4ccccc45)cc3)c3c2oc2c4ccccc4c4ccccc4c23)cc1]

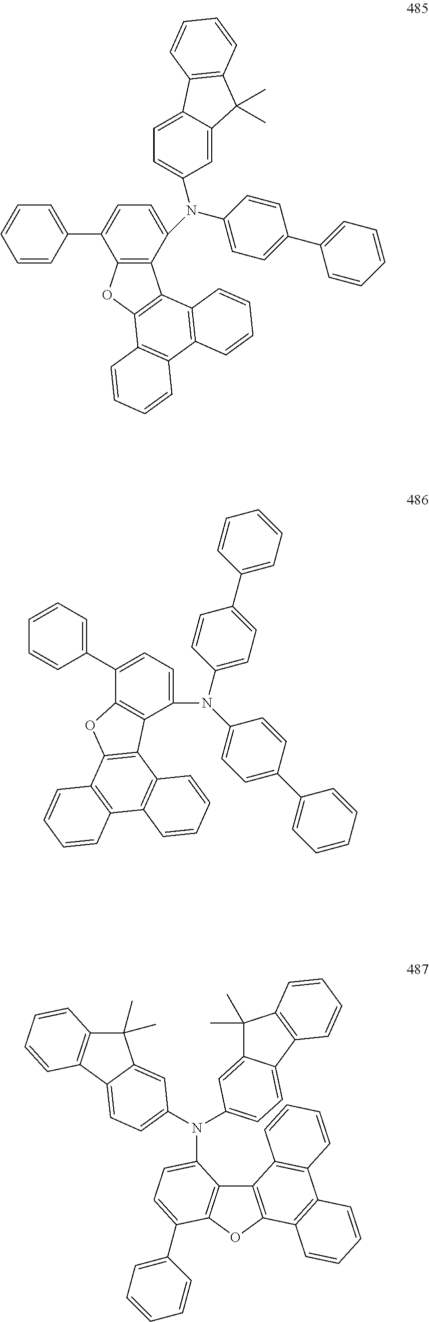 [SMILES: CC1(C)c2ccccc2-c2ccc(N(c3ccc(-c4ccccc4)cc3)c3ccc(-c4ccccc4)c4oc5c6ccccc6c6ccccc6c5c34)cc21 ; c1ccc(-c2ccc(N(c3ccc(-c4ccccc4)cc3)c3ccc(-c4ccccc4)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccccc4)c4oc5c6ccccc6c6ccccc6c5c34)cc21]

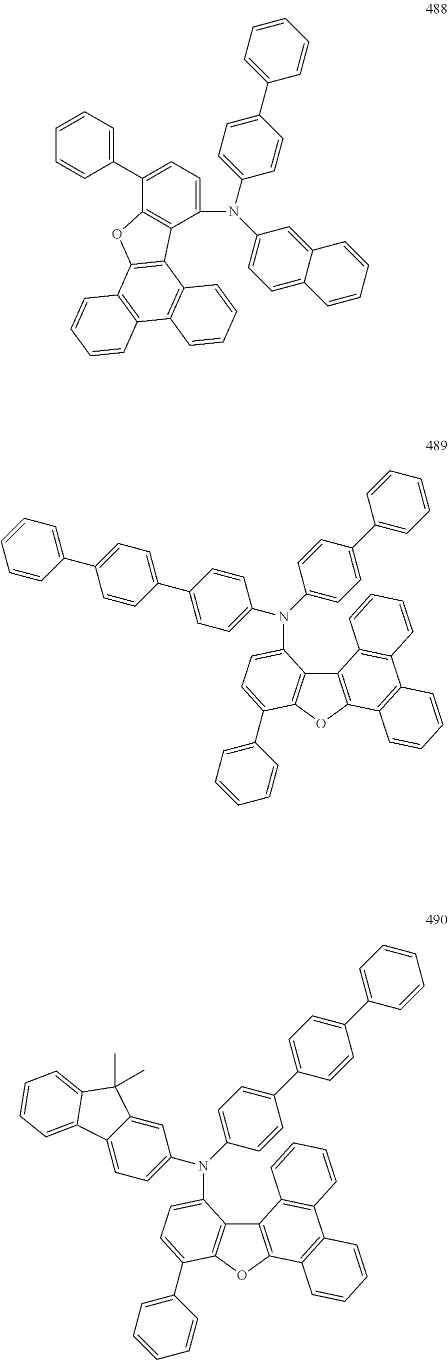 [SMILES: c1ccc(-c2ccc(N(c3ccc4ccccc4c3)c3ccc(-c4ccccc4)c4oc5c6ccccc6c6ccccc6c5c34)cc2)cc1 ; c1ccc(-c2ccc(-c3ccc(N(c4ccc(-c5ccccc5)cc4)c4ccc(-c5ccccc5)c5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3ccc(-c4ccccc4)c4oc5c6ccccc6c6ccccc6c5c34)cc21]

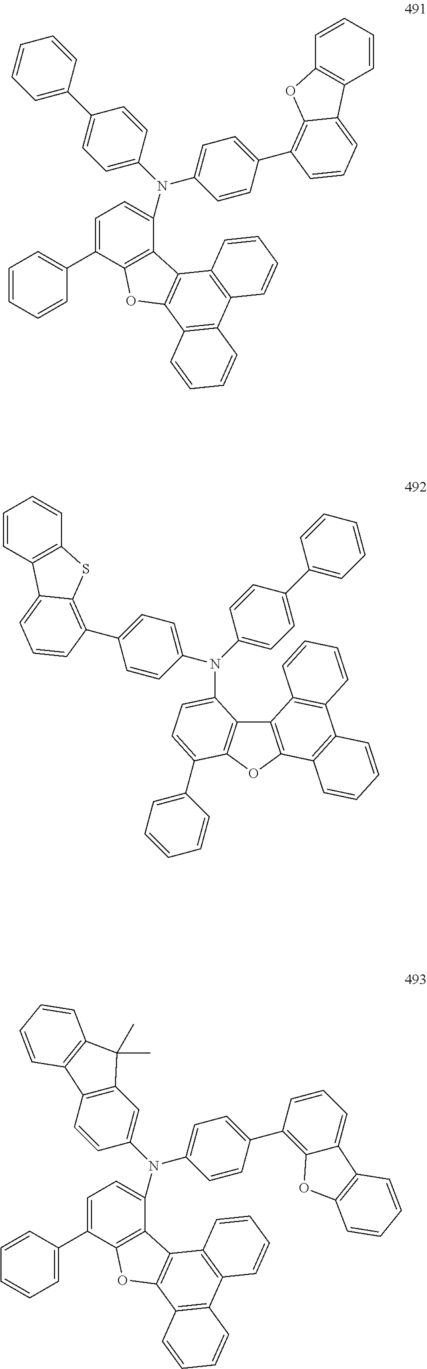 [SMILES: c1ccc(-c2ccc(N(c3ccc(-c4cccc5c4oc4ccccc45)cc3)c3ccc(-c4ccccc4)c4oc5c6ccccc6c6ccccc6c5c34)cc2)cc1 ; c1ccc(-c2ccc(N(c3ccc(-c4cccc5c4sc4ccccc45)cc3)c3ccc(-c4ccccc4)c4oc5c6ccccc6c6ccccc6c5c34)cc2)cc1 ; CC1(C)c2ccccc2-c2ccc(N(c3ccc(-c4cccc5c4oc4ccccc45)cc3)c3ccc(-c4ccccc4)c4oc5c6ccccc6c6ccccc6c5c34)cc21]

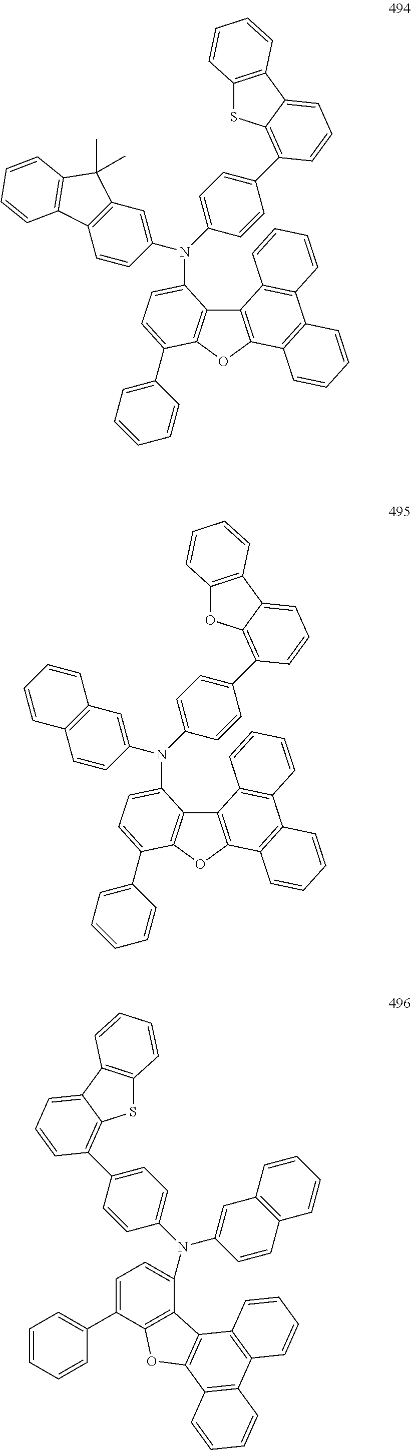 [SMILES: CC1(C)c2ccccc2-c2ccc(N(c3ccc(-c4cccc5c4sc4ccccc45)cc3)c3ccc(-c4ccccc4)c4oc5c6ccccc6c6ccccc6c5c34)cc21 ; c1ccc(-c2ccc(N(c3ccc(-c4cccc5c4oc4ccccc45)cc3)c3ccc4ccccc4c3)c3c2oc2c4ccccc4c4ccccc4c23)cc1 ; c1ccc(-c2ccc(N(c3ccc(-c4cccc5c4sc4ccccc45)cc3)c3ccc4ccccc4c3)c3c2oc2c4ccccc4c4ccccc4c23)cc1]

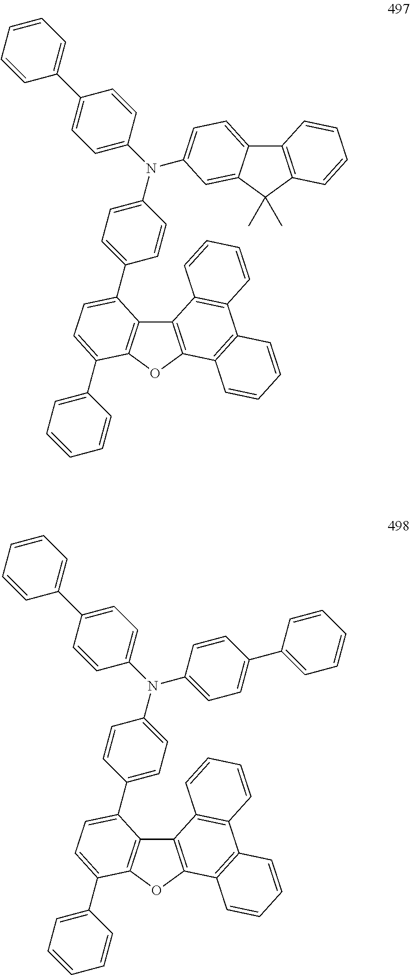 [SMILES: CC1(C)c2ccccc2-c2ccc(N(c3ccc(-c4ccccc4)cc3)c3ccc(-c4ccc(-c5ccccc5)c5oc6c7ccccc7c7ccccc7c6c45)cc3)cc21 ; c1ccc(-c2ccc(N(c3ccc(-c4ccccc4)cc3)c3ccc(-c4ccc(-c5ccccc5)c5oc6c7ccccc7c7ccccc7c6c45)cc3)cc2)cc1]

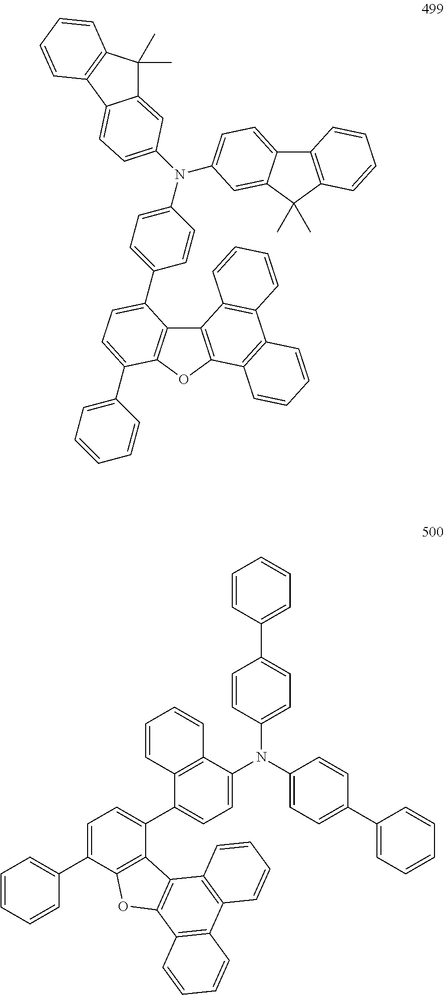 [SMILES: CC1(C)c2ccccc2-c2ccc(N(c3ccc(-c4ccc(-c5ccccc5)c5oc6c7ccccc7c7ccccc7c6c45)cc3)c3ccc4c(c3)C(C)(C)c3ccccc3-4)cc21 ; c1ccc(-c2ccc(N(c3ccc(-c4ccccc4)cc3)c3ccc(-c4ccc(-c5ccccc5)c5oc6c7ccccc7c7ccccc7c6c45)c4ccccc34)cc2)cc1]

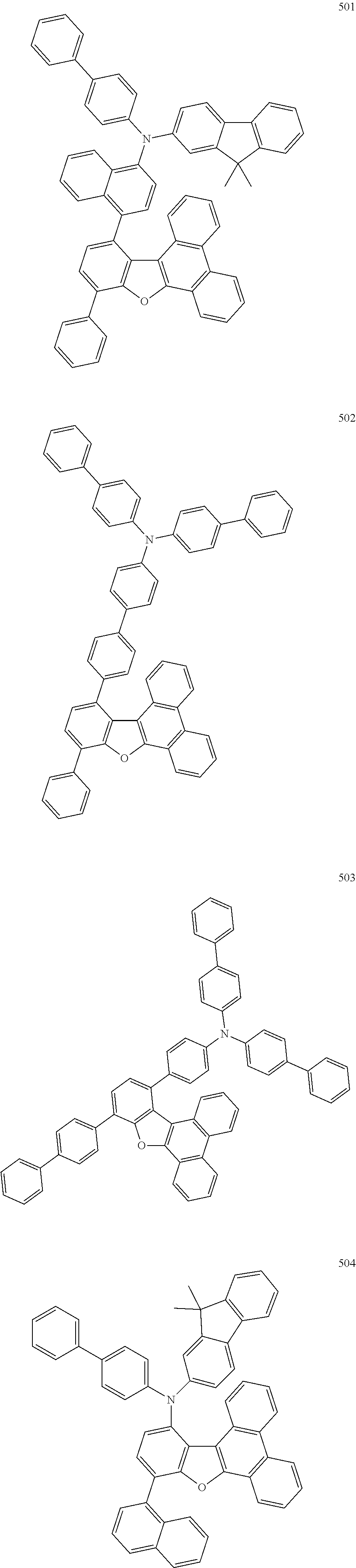 [SMILES: CC1(C)c2ccccc2-c2ccc(N(c3ccc(-c4ccccc4)cc3)c3ccc(-c4ccc(-c5ccccc5)c5oc6c7ccccc7c7ccccc7c6c45)c4ccccc34)cc21 ; c1ccc(-c2ccc(N(c3ccc(-c4ccccc4)cc3)c3ccc(-c4ccc(-c5ccc(-c6ccccc6)c6oc7c8ccccc8c8ccccc8c7c56)cc4)cc3)cc2)cc1 ; c1ccc(-c2ccc(-c3ccc(-c4ccc(N(c5ccc(-c6ccccc6)cc5)c5ccc(-c6ccccc6)cc5)cc4)c4c3oc3c5ccccc5c5ccccc5c34)cc2)cc1 ; CC1(C)c2ccccc2-c2ccc(N(c3ccc(-c4ccccc4)cc3)c3ccc(-c4cccc5ccccc45)c4oc5c6ccccc6c6ccccc6c5c34)cc21]

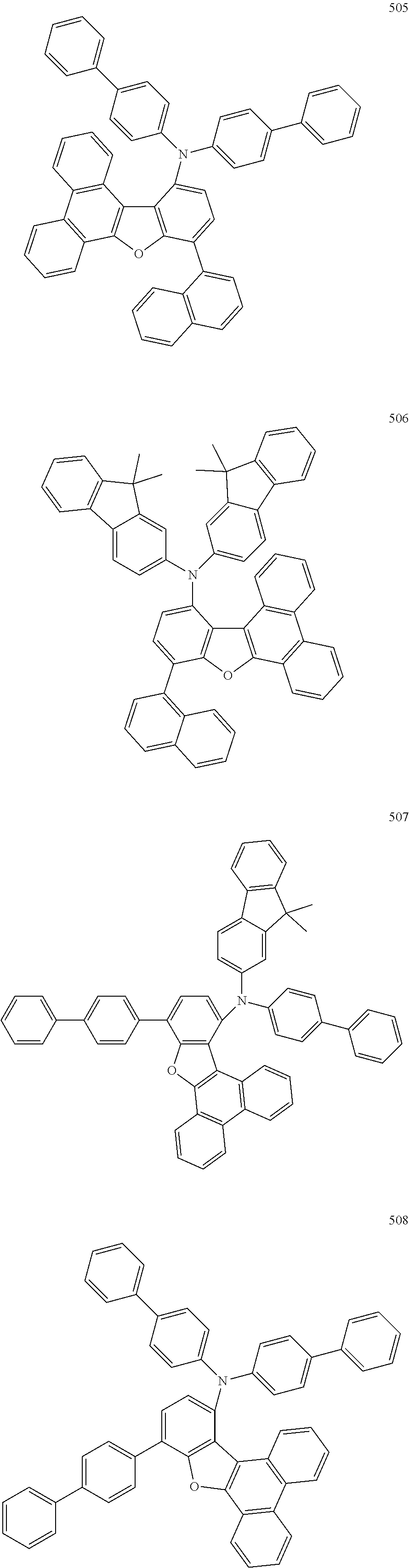 [SMILES: c1ccc(-c2ccc(N(c3ccc(-c4ccccc4)cc3)c3ccc(-c4cccc5ccccc45)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4cccc5ccccc45)c4oc5c6ccccc6c6ccccc6c5c34)cc21 ; CC1(C)c2ccccc2-c2ccc(N(c3ccc(-c4ccccc4)cc3)c3ccc(-c4ccc(-c5ccccc5)cc4)c4oc5c6ccccc6c6ccccc6c5c34)cc21 ; c1ccc(-c2ccc(-c3ccc(N(c4ccc(-c5ccccc5)cc4)c4ccc(-c5ccccc5)cc4)c4c3oc3c5ccccc5c5ccccc5c34)cc2)cc1]

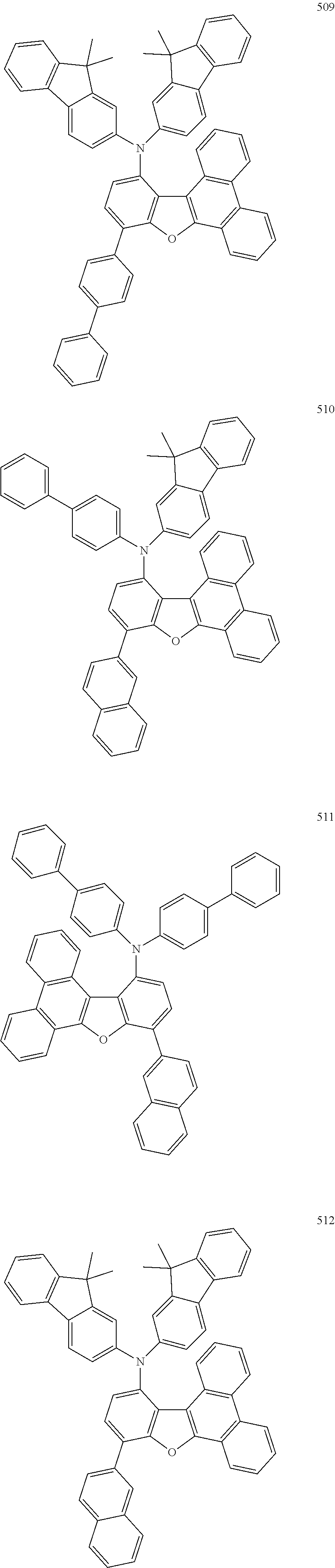 [SMILES: CC1(C)c2ccccc2-c2ccc(N(c3ccc4c(c3)C(C)(C)c3ccccc3-4)c3ccc(-c4ccc(-c5ccccc5)cc4)c4oc5c6ccccc6c6ccccc6c5c34)cc21 ; CC1(C)c2ccccc2-c2ccc(N(c3ccc(-c4ccccc4)cc3)c3ccc(-c4ccc5ccccc5c4)c4oc5c6ccccc6c6ccccc6c5c34)cc21 ; c1ccc(-c2ccc(N(c3ccc(-c4ccccc4)cc3)c3ccc(-c4ccc5ccccc5c4)c4oc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccc5ccccc5c4)c4oc5c6ccccc6c6ccccc6c5c34)cc21]

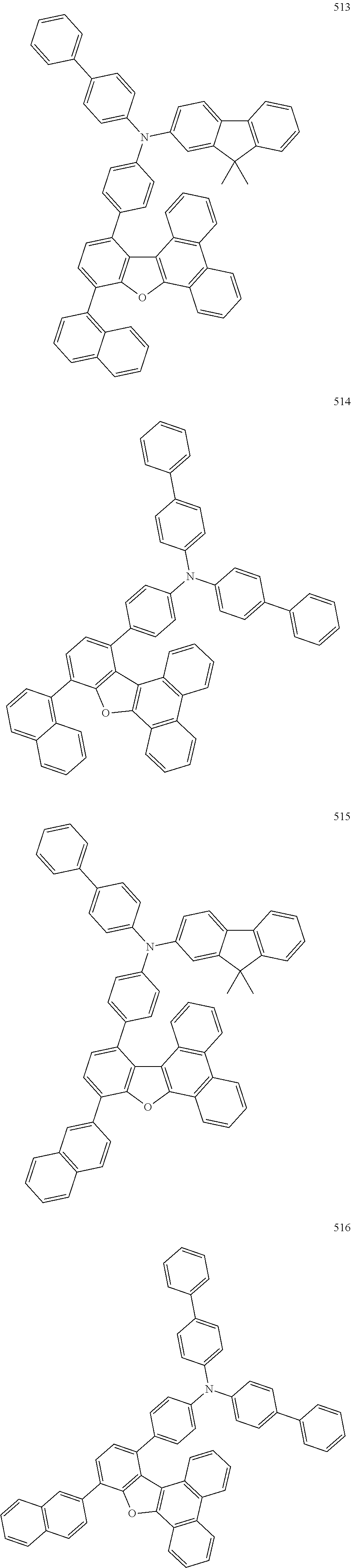 [SMILES: CC1(C)c2ccccc2-c2ccc(N(c3ccc(-c4ccccc4)cc3)c3ccc(-c4ccc(-c5cccc6ccccc56)c5oc6c7ccccc7c7ccccc7c6c45)cc3)cc21 ; c1ccc(-c2ccc(N(c3ccc(-c4ccccc4)cc3)c3ccc(-c4ccc(-c5cccc6ccccc56)c5oc6c7ccccc7c7ccccc7c6c45)cc3)cc2)cc1 ; CC1(C)c2ccccc2-c2ccc(N(c3ccc(-c4ccccc4)cc3)c3ccc(-c4ccc(-c5ccc6ccccc6c5)c5oc6c7ccccc7c7ccccc7c6c45)cc3)cc21 ; c1ccc(-c2ccc(N(c3ccc(-c4ccccc4)cc3)c3ccc(-c4ccc(-c5ccc6ccccc6c5)c5oc6c7ccccc7c7ccccc7c6c45)cc3)cc2)cc1]

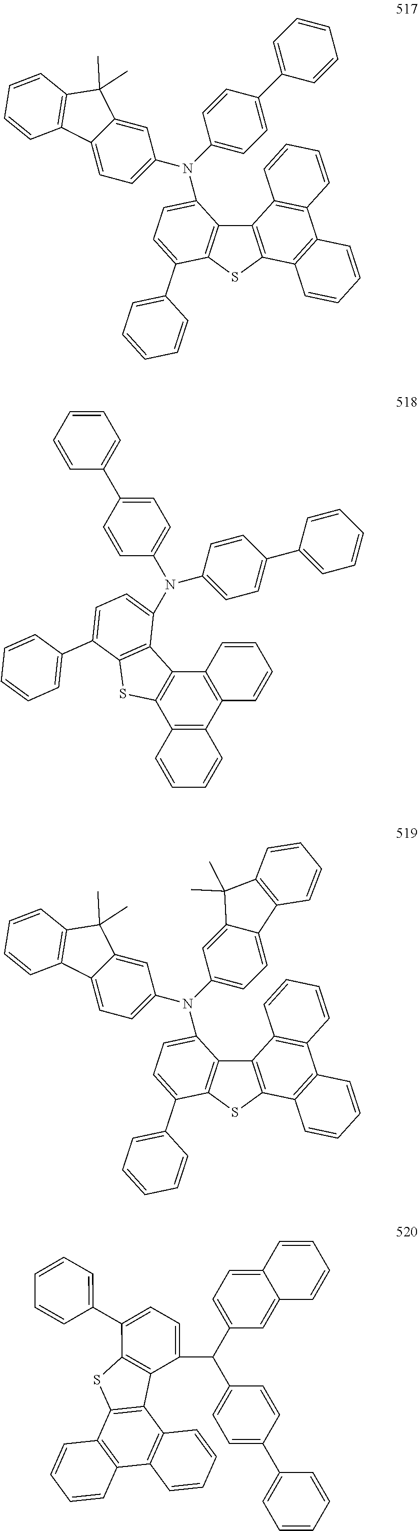 [SMILES: CC1(C)c2ccccc2-c2ccc(N(c3ccc(-c4ccccc4)cc3)c3ccc(-c4ccccc4)c4sc5c6ccccc6c6ccccc6c5c34)cc21 ; c1ccc(-c2ccc(N(c3ccc(-c4ccccc4)cc3)c3ccc(-c4ccccc4)c4sc5c6ccccc6c6ccccc6c5c34)cc2)cc1 ; CC1(C)c2ccccc2-c2ccc(N(c3ccc4c(c3)C(C)(C)c3ccccc3-4)c3ccc(-c4ccccc4)c4sc5c6ccccc6c6ccccc6c5c34)cc21 ; c1ccc(-c2ccc(C(c3ccc4ccccc4c3)c3ccc(-c4ccccc4)c4sc5c6ccccc6c6ccccc6c5c34)cc2)cc1]

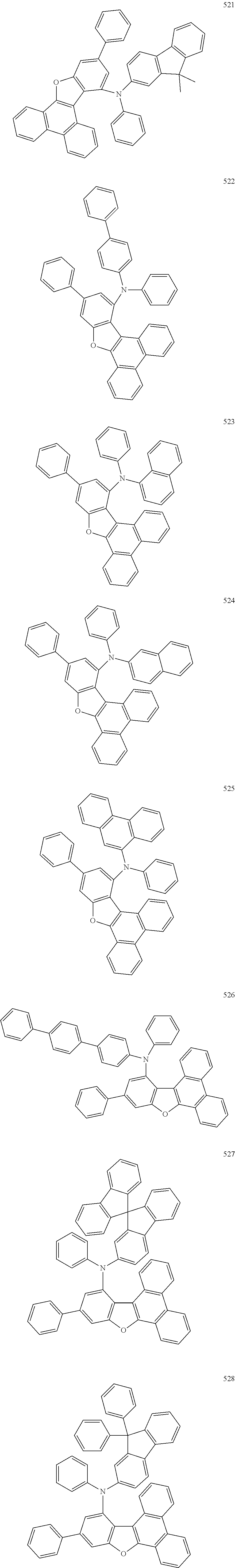

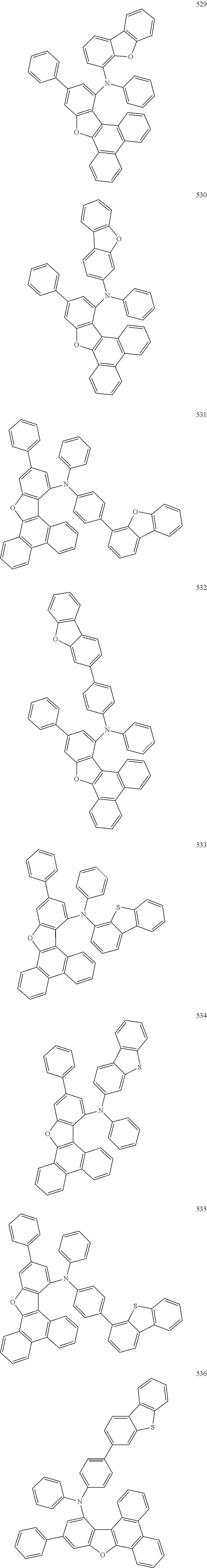

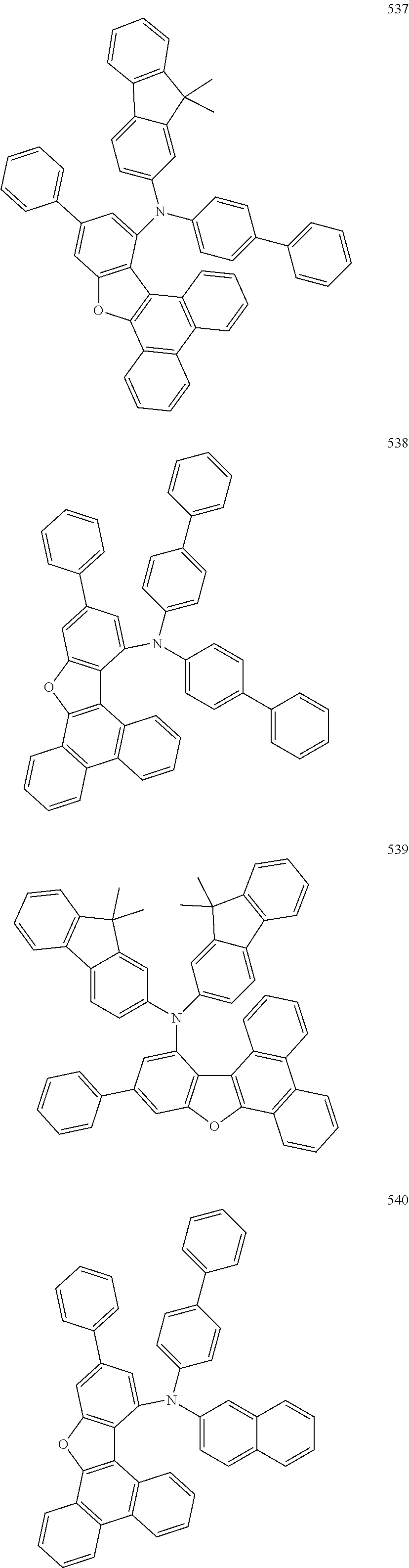 [SMILES: CC1(C)c2ccccc2-c2ccc(N(c3ccc(-c4ccccc4)cc3)c3cc(-c4ccccc4)cc4oc5c6ccccc6c6ccccc6c5c34)cc21 ; c1ccc(-c2ccc(N(c3ccc(-c4ccccc4)cc3)c3cc(-c4ccccc4)cc4oc5c6ccccc6c6ccccc6c5c34)cc2)cc1 ; CC1(C)c2ccccc2-c2ccc(N(c3ccc4c(c3)C(C)(C)c3ccccc3-4)c3cc(-c4ccccc4)cc4oc5c6ccccc6c6ccccc6c5c34)cc21 ; c1ccc(-c2ccc(N(c3ccc4ccccc4c3)c3cc(-c4ccccc4)cc4oc5c6ccccc6c6ccccc6c5c34)cc2)cc1]

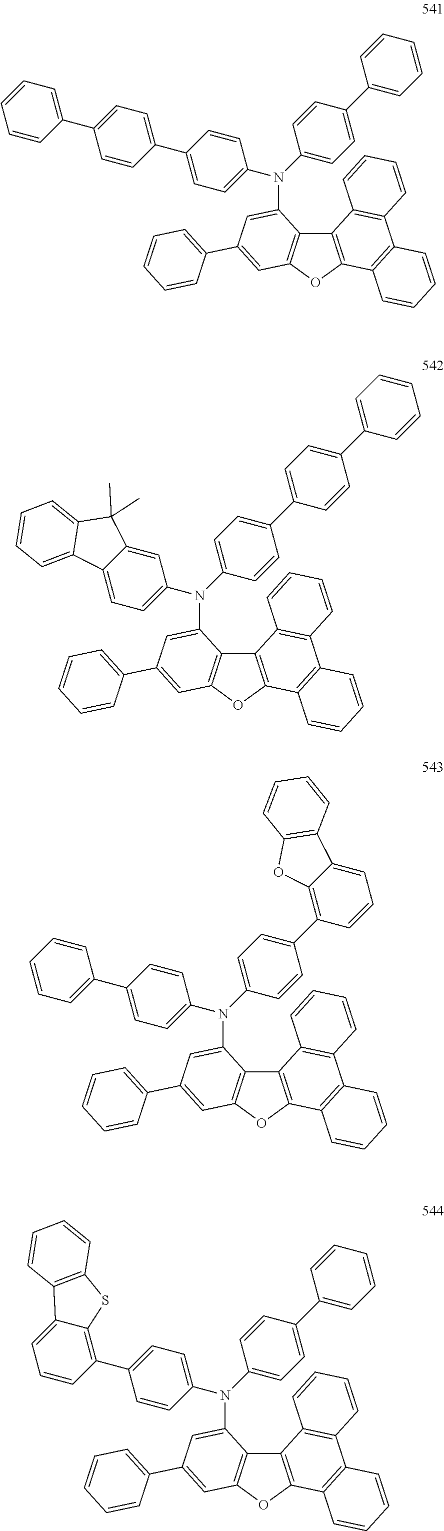 [SMILES: c1ccc(-c2ccc(-c3ccc(N(c4ccc(-c5ccccc5)cc4)c4cc(-c5ccccc5)cc5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3cc(-c4ccccc4)cc4oc5c6ccccc6c6ccccc6c5c34)cc21 ; c1ccc(-c2ccc(N(c3ccc(-c4cccc5c4oc4ccccc45)cc3)c3cc(-c4ccccc4)cc4oc5c6ccccc6c6ccccc6c5c34)cc2)cc1 ; c1ccc(-c2ccc(N(c3ccc(-c4cccc5c4sc4ccccc45)cc3)c3cc(-c4ccccc4)cc4oc5c6ccccc6c6ccccc6c5c34)cc2)cc1]

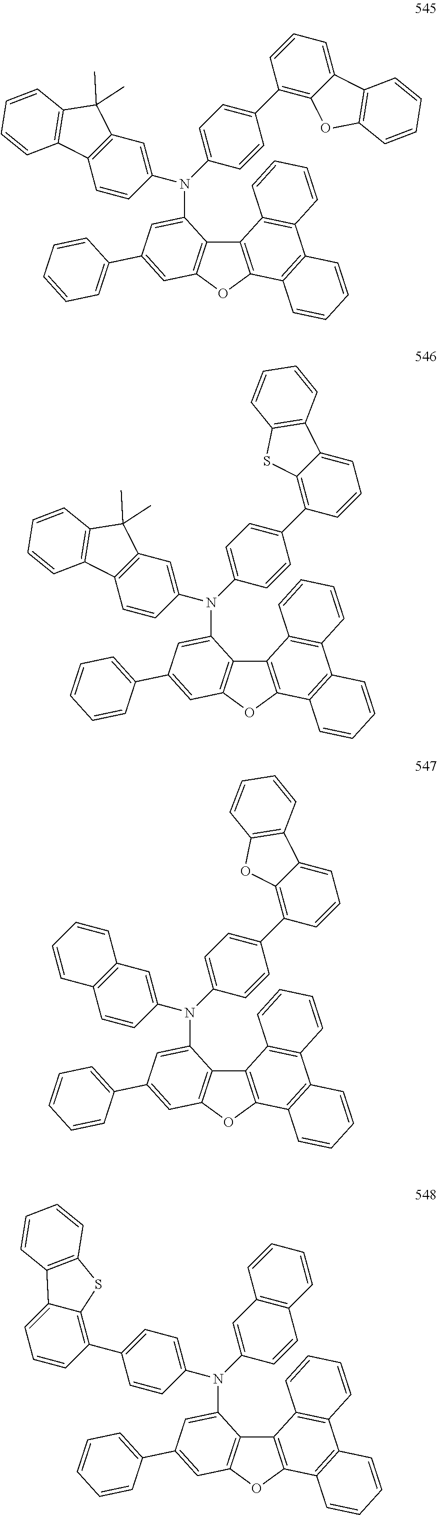 [SMILES: CC1(C)c2ccccc2-c2ccc(N(c3ccc(-c4cccc5c4oc4ccccc45)cc3)c3cc(-c4ccccc4)cc4oc5c6ccccc6c6ccccc6c5c34)cc21 ; CC1(C)c2ccccc2-c2ccc(N(c3ccc(-c4cccc5c4sc4ccccc45)cc3)c3cc(-c4ccccc4)cc4oc5c6ccccc6c6ccccc6c5c34)cc21 ; c1ccc(-c2cc(N(c3ccc(-c4cccc5c4oc4ccccc45)cc3)c3ccc4ccccc4c3)c3c(c2)oc2c4ccccc4c4ccccc4c23)cc1 ; c1ccc(-c2cc(N(c3ccc(-c4cccc5c4sc4ccccc45)cc3)c3ccc4ccccc4c3)c3c(c2)oc2c4ccccc4c4ccccc4c23)cc1]

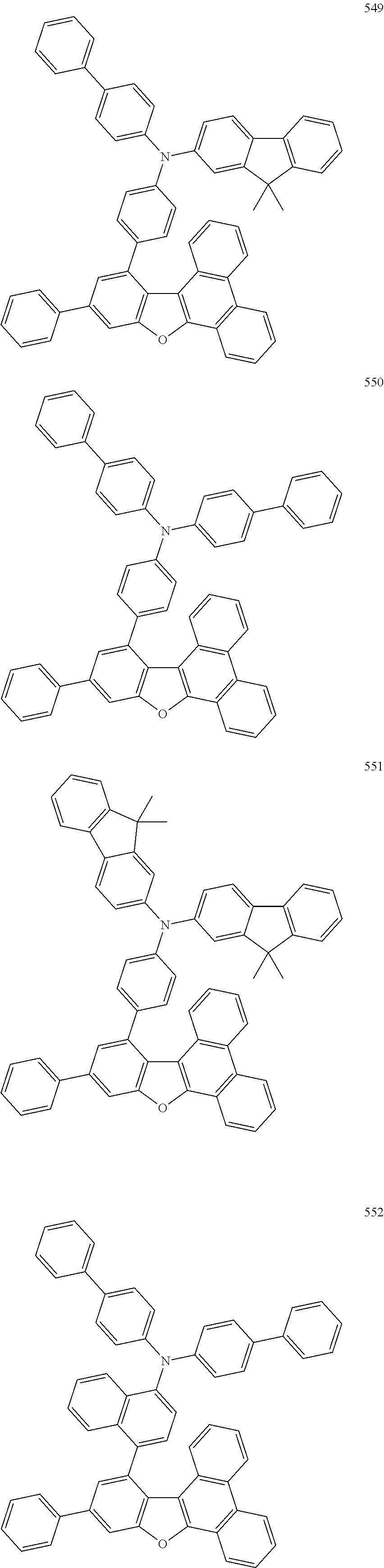 [SMILES: CC1(C)c2ccccc2-c2ccc(N(c3ccc(-c4ccccc4)cc3)c3ccc(-c4cc(-c5ccccc5)cc5oc6c7ccccc7c7ccccc7c6c45)cc3)cc21 ; c1ccc(-c2ccc(N(c3ccc(-c4ccccc4)cc3)c3ccc(-c4cc(-c5ccccc5)cc5oc6c7ccccc7c7ccccc7c6c45)cc3)cc2)cc1 ; CC1(C)c2ccccc2-c2ccc(N(c3ccc(-c4cc(-c5ccccc5)cc5oc6c7ccccc7c7ccccc7c6c45)cc3)c3ccc4c(c3)C(C)(C)c3ccccc3-4)cc21 ; c1ccc(-c2ccc(N(c3ccc(-c4ccccc4)cc3)c3ccc(-c4cc(-c5ccccc5)cc5oc6c7ccccc7c7ccccc7c6c45)c4ccccc34)cc2)cc1]

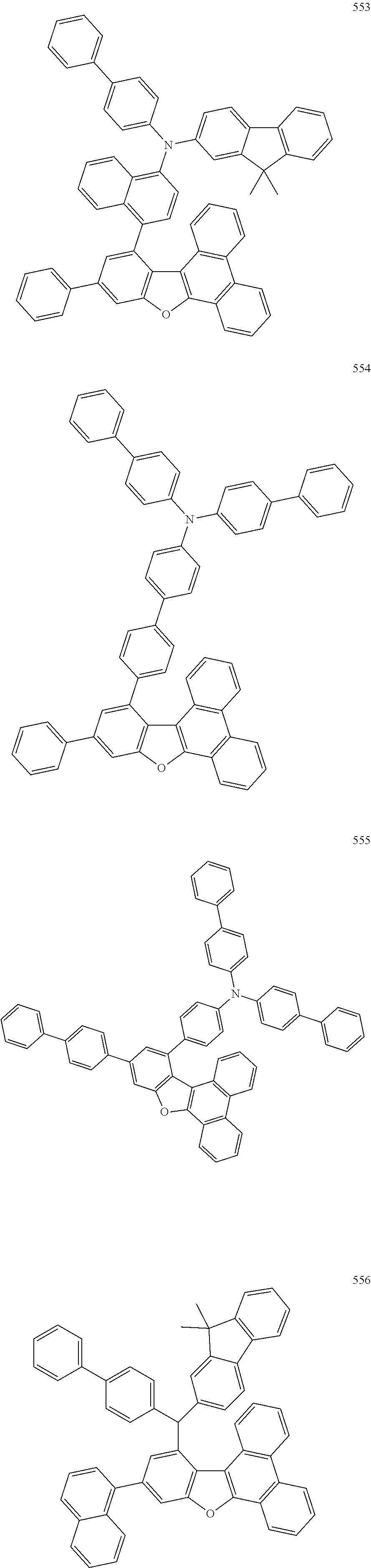 [SMILES: CC1(C)c2ccccc2-c2ccc(N(c3ccc(-c4ccccc4)cc3)c3ccc(-c4cc(-c5ccccc5)cc5oc6c7ccccc7c7ccccc7c6c45)c4ccccc34)cc21 ; c1ccc(-c2ccc(N(c3ccc(-c4ccccc4)cc3)c3ccc(-c4ccc(-c5cc(-c6ccccc6)cc6oc7c8ccccc8c8ccccc8c7c56)cc4)cc3)cc2)cc1 ; c1ccc(-c2ccc(-c3cc(-c4ccc(N(c5ccc(-c6ccccc6)cc5)c5ccc(-c6ccccc6)cc5)cc4)c4c(c3)oc3c5ccccc5c5ccccc5c34)cc2)cc1 ; CC1(C)c2ccccc2-c2ccc(C(c3ccc(-c4ccccc4)cc3)c3cc(-c4cccc5ccccc45)cc4oc5c6ccccc6c6ccccc6c5c34)cc21]

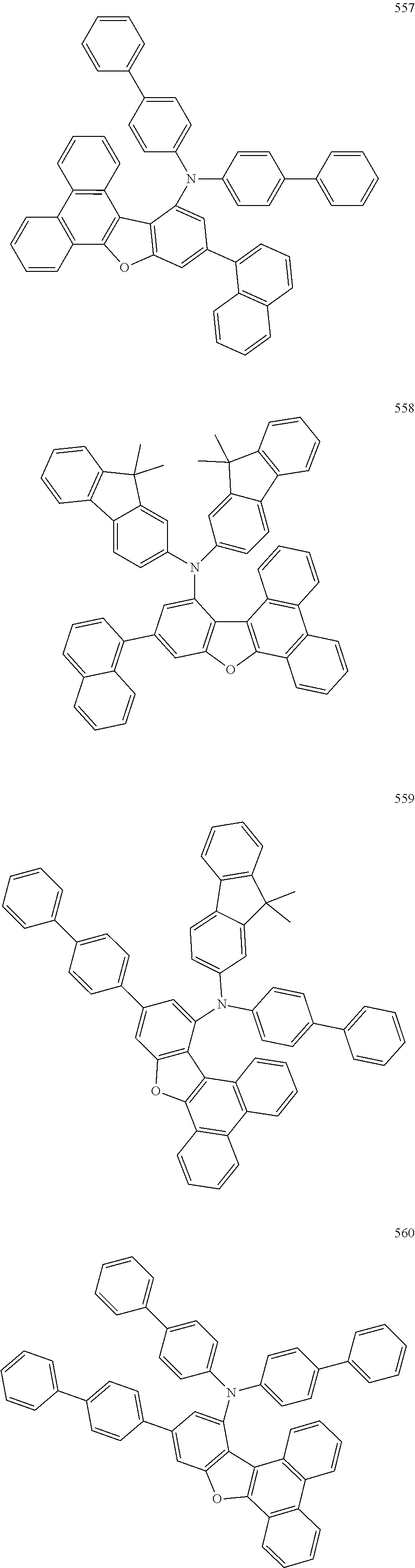 [SMILES: c1ccc(-c2ccc(N(c3ccc(-c4ccccc4)cc3)c3cc(-c4cccc5ccccc45)cc4oc5c6ccccc6c6ccccc6c5c34)cc2)cc1 ; CC1(C)c2ccccc2-c2ccc(N(c3ccc4c(c3)C(C)(C)c3ccccc3-4)c3cc(-c4cccc5ccccc45)cc4oc5c6ccccc6c6ccccc6c5c34)cc21 ; CC1(C)c2ccccc2-c2ccc(N(c3ccc(-c4ccccc4)cc3)c3cc(-c4ccc(-c5ccccc5)cc4)cc4oc5c6ccccc6c6ccccc6c5c34)cc21 ; c1ccc(-c2ccc(-c3cc(N(c4ccc(-c5ccccc5)cc4)c4ccc(-c5ccccc5)cc4)c4c(c3)oc3c5ccccc5c5ccccc5c34)cc2)cc1]

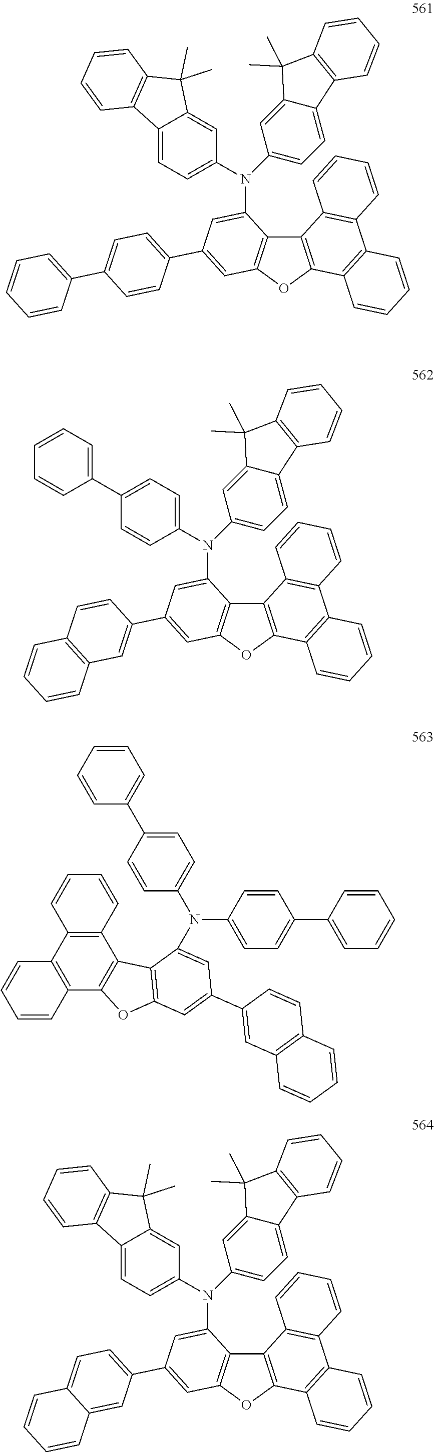

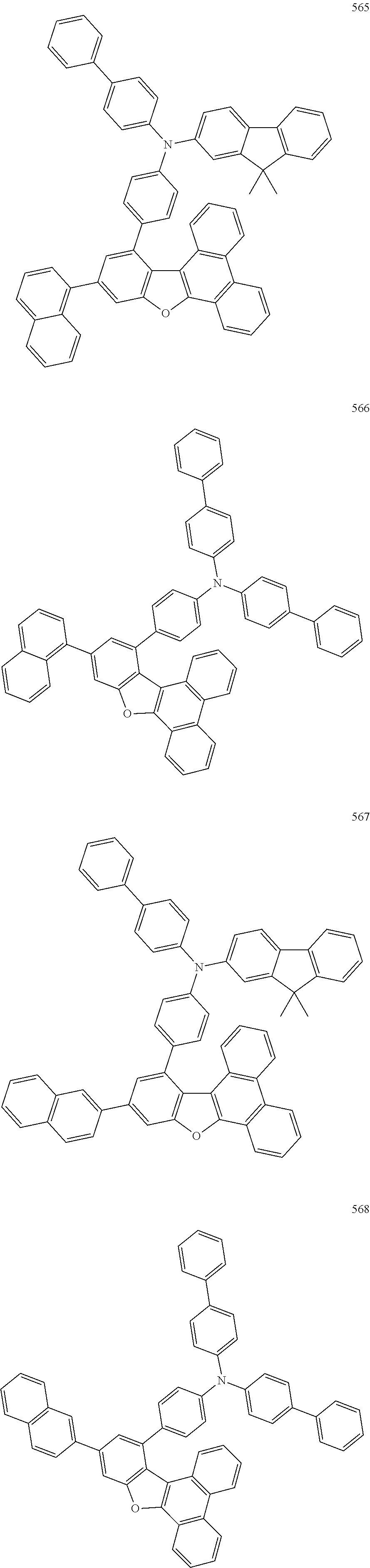 [SMILES: CC1(C)c2ccccc2-c2ccc(N(c3ccc(-c4ccccc4)cc3)c3ccc(-c4cc(-c5cccc6ccccc56)cc5oc6c7ccccc7c7ccccc7c6c45)cc3)cc21 ; c1ccc(-c2ccc(N(c3ccc(-c4ccccc4)cc3)c3ccc(-c4cc(-c5cccc6ccccc56)cc5oc6c7ccccc7c7ccccc7c6c45)cc3)cc2)cc1 ; CC1(C)c2ccccc2-c2ccc(N(c3ccc(-c4ccccc4)cc3)c3ccc(-c4cc(-c5ccc6ccccc6c5)cc5oc6c7ccccc7c7ccccc7c6c45)cc3)cc21 ; c1ccc(-c2ccc(N(c3ccc(-c4ccccc4)cc3)c3ccc(-c4cc(-c5ccc6ccccc6c5)cc5oc6c7ccccc7c7ccccc7c6c45)cc3)cc2)cc1]

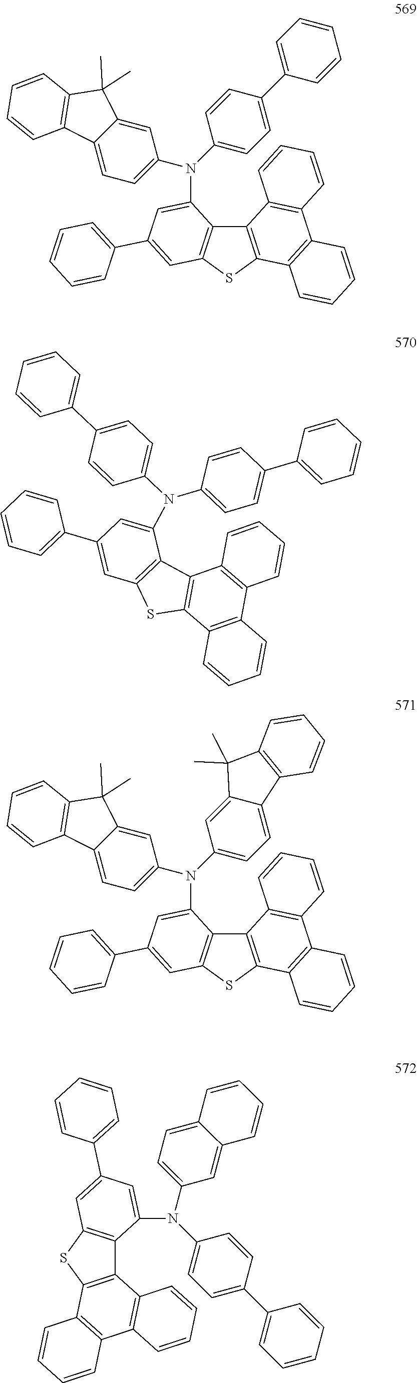 [SMILES: CC1(C)c2ccccc2-c2ccc(N(c3ccc(-c4ccccc4)cc3)c3cc(-c4ccccc4)cc4sc5c6ccccc6c6ccccc6c5c34)cc21 ; c1ccc(-c2ccc(N(c3ccc(-c4ccccc4)cc3)c3cc(-c4ccccc4)cc4sc5c6ccccc6c6ccccc6c5c34)cc2)cc1 ; CC1(C)c2ccccc2-c2ccc(N(c3ccc4c(c3)C(C)(C)c3ccccc3-4)c3cc(-c4ccccc4)cc4sc5c6ccccc6c6ccccc6c5c34)cc21 ; c1ccc(-c2ccc(N(c3ccc4ccccc4c3)c3cc(-c4ccccc4)cc4sc5c6ccccc6c6ccccc6c5c34)cc2)cc1]

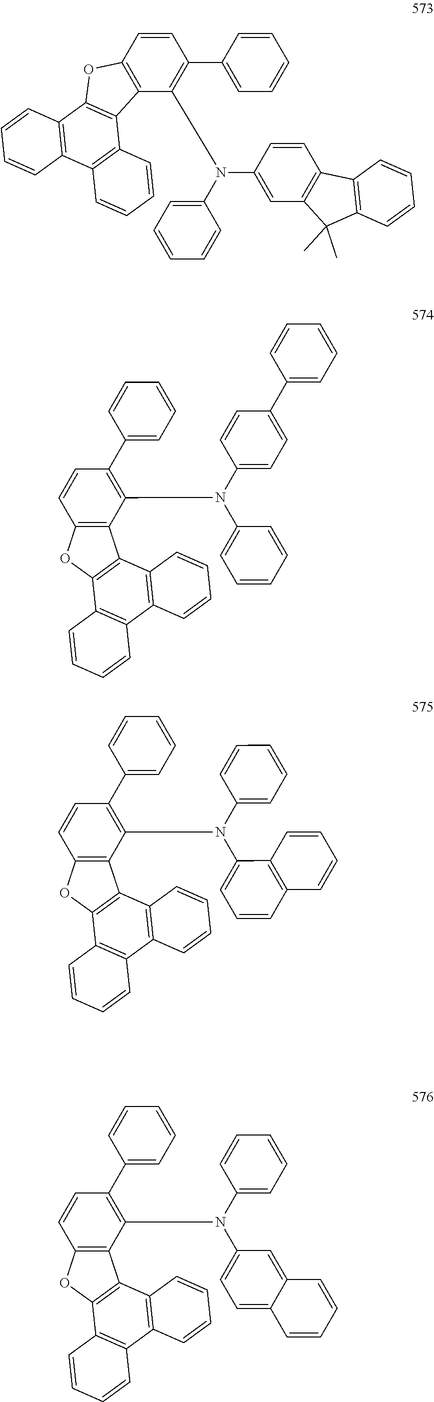

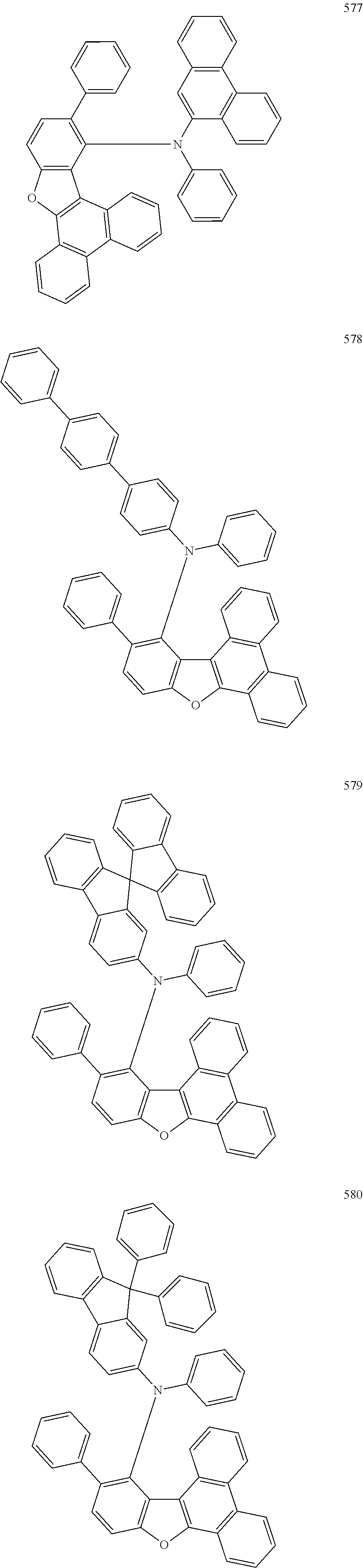 [SMILES: c1ccc(-c2ccc3oc4c5ccccc5c5ccccc5c4c3c2N(c2ccccc2)c2cc3ccccc3c3ccccc23)cc1 ; c1ccc(-c2ccc(-c3ccc(N(c4ccccc4)c4c(-c5ccccc5)ccc5oc6c7ccccc7c7ccccc7c6c45)cc3)cc2)cc1 ; c1ccc(-c2ccc3oc4c5ccccc5c5ccccc5c4c3c2N(c2ccccc2)c2ccc3c(c2)C2(c4ccccc4-c4ccccc42)c2ccccc2-3)cc1 ; c1ccc(-c2ccc3oc4c5ccccc5c5ccccc5c4c3c2N(c2ccccc2)c2ccc3c(c2)C(c2ccccc2)(c2ccccc2)c2ccccc2-3)cc1]

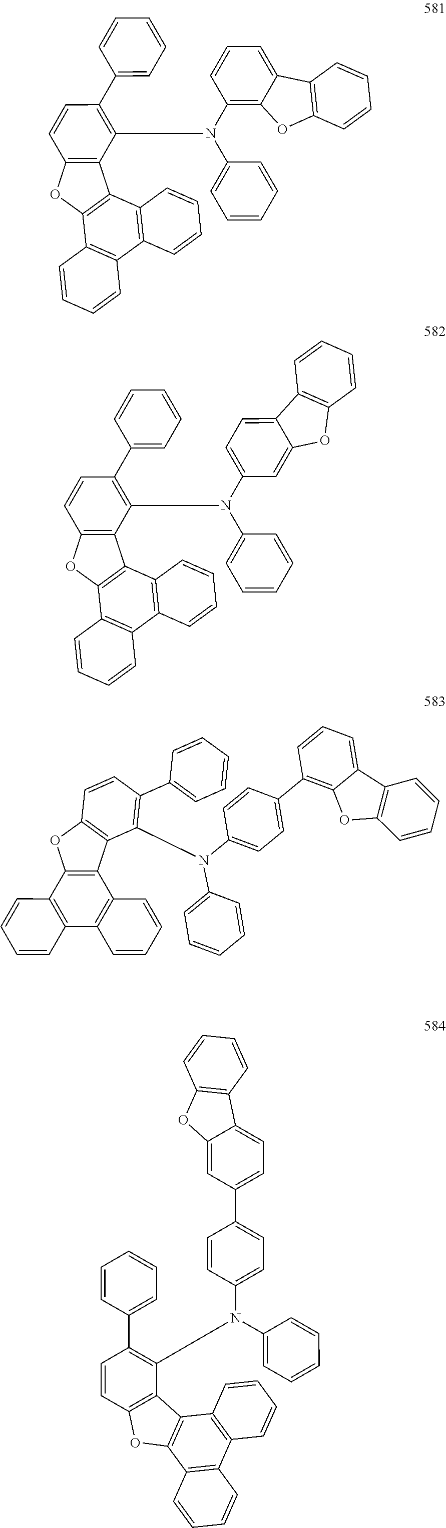 [SMILES: c1ccc(-c2ccc3oc4c5ccccc5c5ccccc5c4c3c2N(c2ccccc2)c2cccc3c2oc2ccccc23)cc1 ; c1ccc(-c2ccc3oc4c5ccccc5c5ccccc5c4c3c2N(c2ccccc2)c2ccc3c(c2)oc2ccccc23)cc1 ; c1ccc(-c2ccc3oc4c5ccccc5c5ccccc5c4c3c2N(c2ccccc2)c2ccc(-c3cccc4c3oc3ccccc34)cc2)cc1 ; c1ccc(-c2ccc3oc4c5ccccc5c5ccccc5c4c3c2N(c2ccccc2)c2ccc(-c3ccc4c(c3)oc3ccccc34)cc2)cc1]

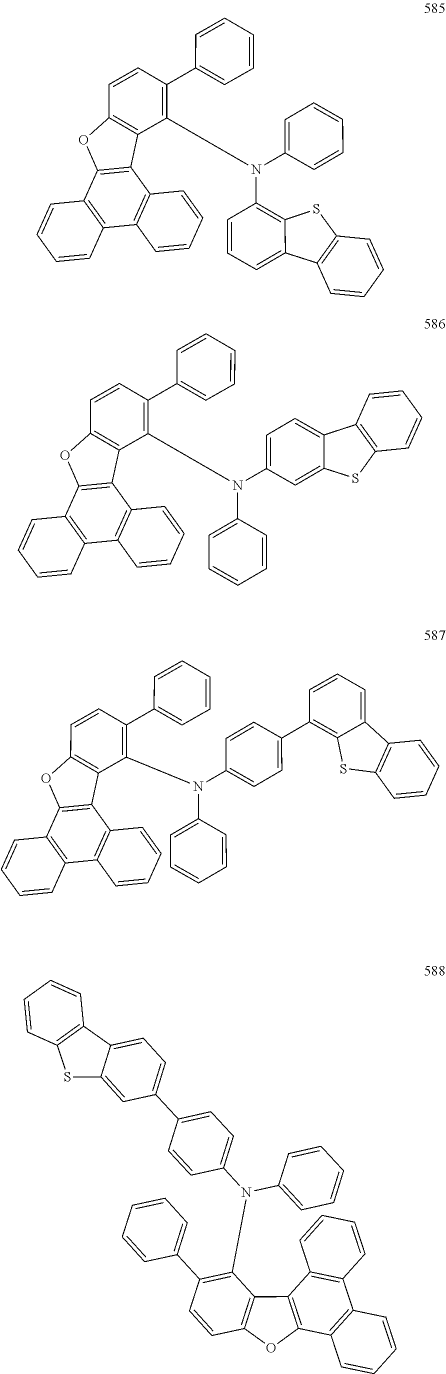 [SMILES: c1ccc(-c2ccc3oc4c5ccccc5c5ccccc5c4c3c2N(c2ccccc2)c2cccc3c2sc2ccccc23)cc1 ; c1ccc(-c2ccc3oc4c5ccccc5c5ccccc5c4c3c2N(c2ccccc2)c2ccc3c(c2)sc2ccccc23)cc1 ; c1ccc(-c2ccc3oc4c5ccccc5c5ccccc5c4c3c2N(c2ccccc2)c2ccc(-c3cccc4c3sc3ccccc34)cc2)cc1 ; c1ccc(-c2ccc3oc4c5ccccc5c5ccccc5c4c3c2N(c2ccccc2)c2ccc(-c3ccc4c(c3)sc3ccccc34)cc2)cc1]

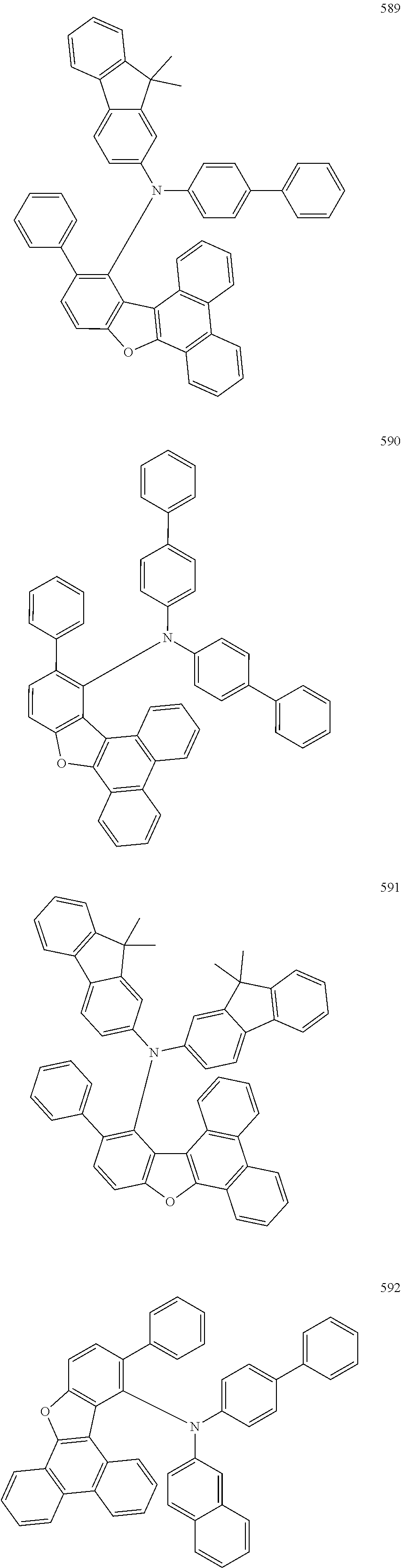 [SMILES: CC1(C)c2ccccc2-c2ccc(N(c3ccc(-c4ccccc4)cc3)c3c(-c4ccccc4)ccc4oc5c6ccccc6c6ccccc6c5c34)cc21 ; c1ccc(-c2ccc(N(c3ccc(-c4ccccc4)cc3)c3c(-c4ccccc4)ccc4oc5c6ccccc6c6ccccc6c5c34)cc2)cc1 ; CC1(C)c2ccccc2-c2ccc(N(c3ccc4c(c3)C(C)(C)c3ccccc3-4)c3c(-c4ccccc4)ccc4oc5c6ccccc6c6ccccc6c5c34)cc21 ; c1ccc(-c2ccc(N(c3ccc4ccccc4c3)c3c(-c4ccccc4)ccc4oc5c6ccccc6c6ccccc6c5c34)cc2)cc1]

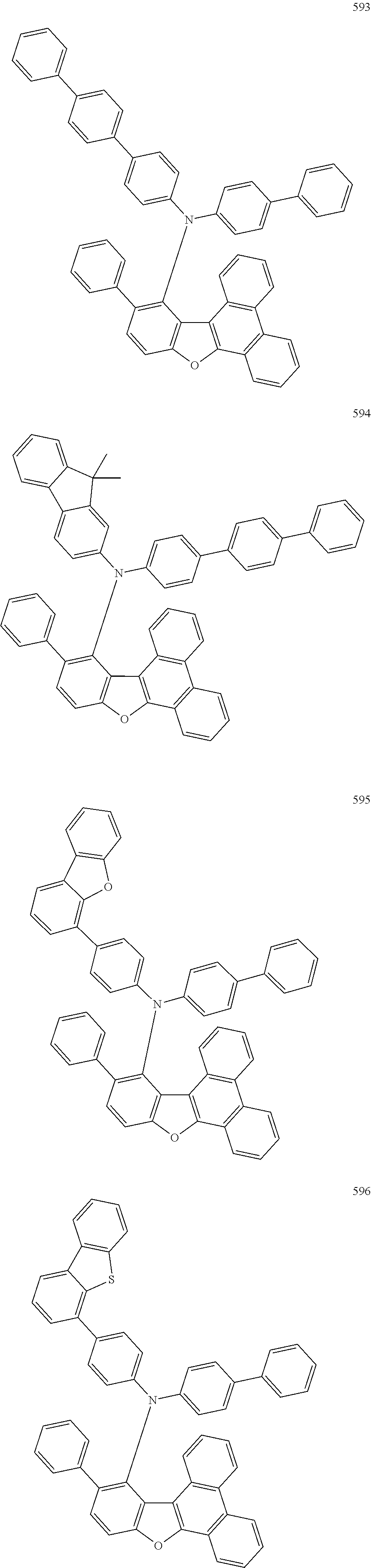 [SMILES: c1ccc(-c2ccc(-c3ccc(N(c4ccc(-c5ccccc5)cc4)c4c(-c5ccccc5)ccc5oc6c7ccccc7c7ccccc7c6c45)cc3)cc2)cc1 ; CC1(C)c2ccccc2-c2ccc(N(c3ccc(-c4ccc(-c5ccccc5)cc4)cc3)c3c(-c4ccccc4)ccc4oc5c6ccccc6c6ccccc6c5c34)cc21 ; c1ccc(-c2ccc(N(c3ccc(-c4cccc5c4oc4ccccc45)cc3)c3c(-c4ccccc4)ccc4oc5c6ccccc6c6ccccc6c5c34)cc2)cc1 ; c1ccc(-c2ccc(N(c3ccc(-c4cccc5c4sc4ccccc45)cc3)c3c(-c4ccccc4)ccc4oc5c6ccccc6c6ccccc6c5c34)cc2)cc1]

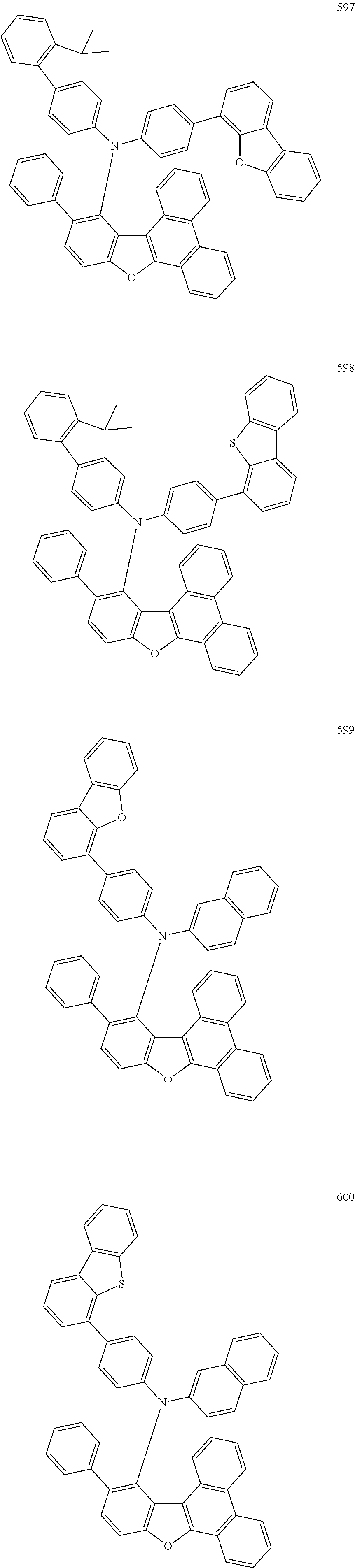 [SMILES: CC1(C)c2ccccc2-c2ccc(N(c3ccc(-c4cccc5c4oc4ccccc45)cc3)c3c(-c4ccccc4)ccc4oc5c6ccccc6c6ccccc6c5c34)cc21 ; CC1(C)c2ccccc2-c2ccc(N(c3ccc(-c4cccc5c4sc4ccccc45)cc3)c3c(-c4ccccc4)ccc4oc5c6ccccc6c6ccccc6c5c34)cc21 ; c1ccc(-c2ccc3oc4c5ccccc5c5ccccc5c4c3c2N(c2ccc(-c3cccc4c3oc3ccccc34)cc2)c2ccc3ccccc3c2)cc1 ; c1ccc(-c2ccc3oc4c5ccccc5c5ccccc5c4c3c2N(c2ccc(-c3cccc4c3sc3ccccc34)cc2)c2ccc3ccccc3c2)cc1]

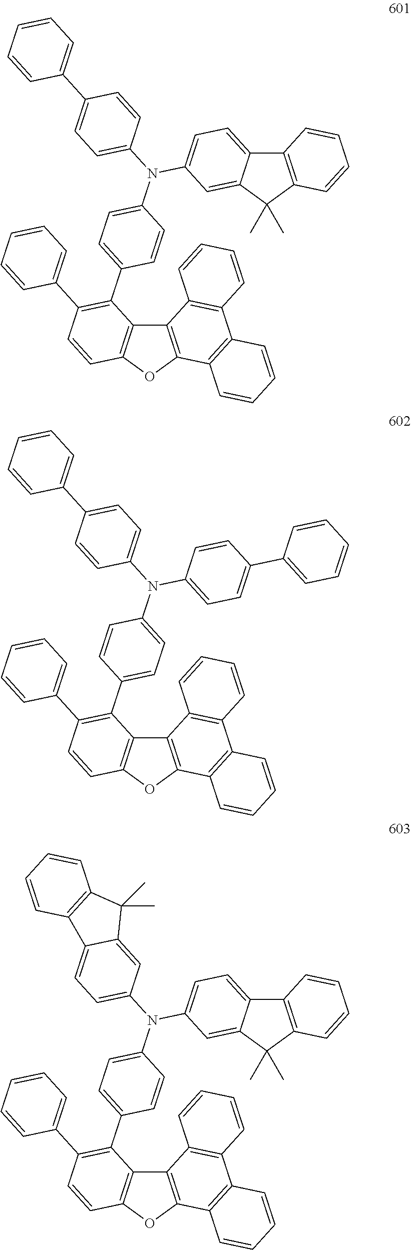 [SMILES: CC1(C)c2ccccc2-c2ccc(N(c3ccc(-c4ccccc4)cc3)c3ccc(-c4c(-c5ccccc5)ccc5oc6c7ccccc7c7ccccc7c6c45)cc3)cc21 ; c1ccc(-c2ccc(N(c3ccc(-c4ccccc4)cc3)c3ccc(-c4c(-c5ccccc5)ccc5oc6c7ccccc7c7ccccc7c6c45)cc3)cc2)cc1 ; CC1(C)c2ccccc2-c2ccc(N(c3ccc(-c4c(-c5ccccc5)ccc5oc6c7ccccc7c7ccccc7c6c45)cc3)c3ccc4c(c3)C(C)(C)c3ccccc3-4)cc21]

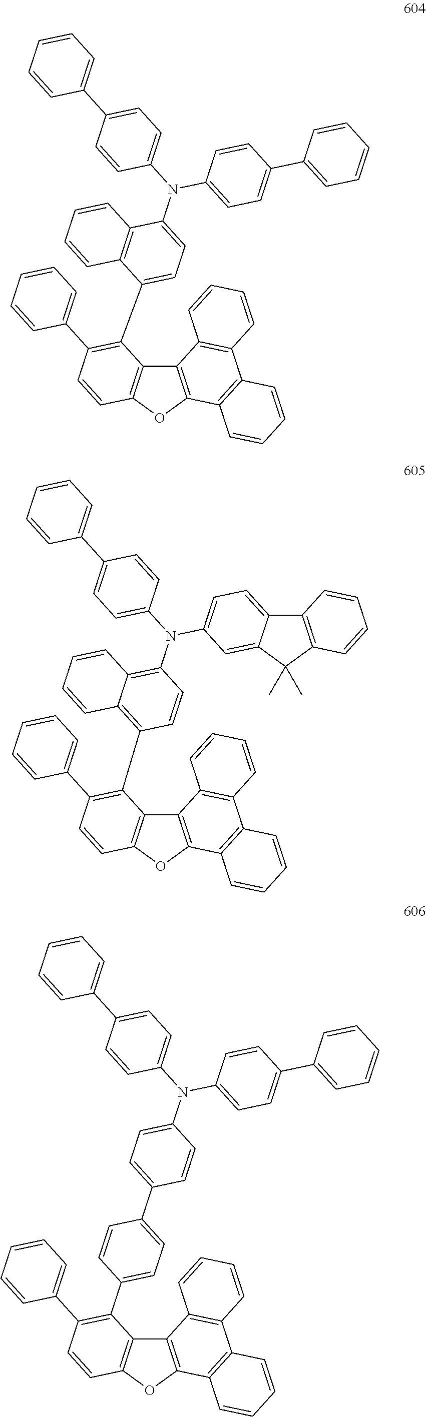 [SMILES: c1ccc(-c2ccc(N(c3ccc(-c4ccccc4)cc3)c3ccc(-c4c(-c5ccccc5)ccc5oc6c7ccccc7c7ccccc7c6c45)c4ccccc34)cc2)cc1 ; CC1(C)c2ccccc2-c2ccc(N(c3ccc(-c4ccccc4)cc3)c3ccc(-c4c(-c5ccccc5)ccc5oc6c7ccccc7c7ccccc7c6c45)c4ccccc34)cc21 ; c1ccc(-c2ccc(N(c3ccc(-c4ccccc4)cc3)c3ccc(-c4ccc(-c5c(-c6ccccc6)ccc6oc7c8ccccc8c8ccccc8c7c56)cc4)cc3)cc2)cc1]

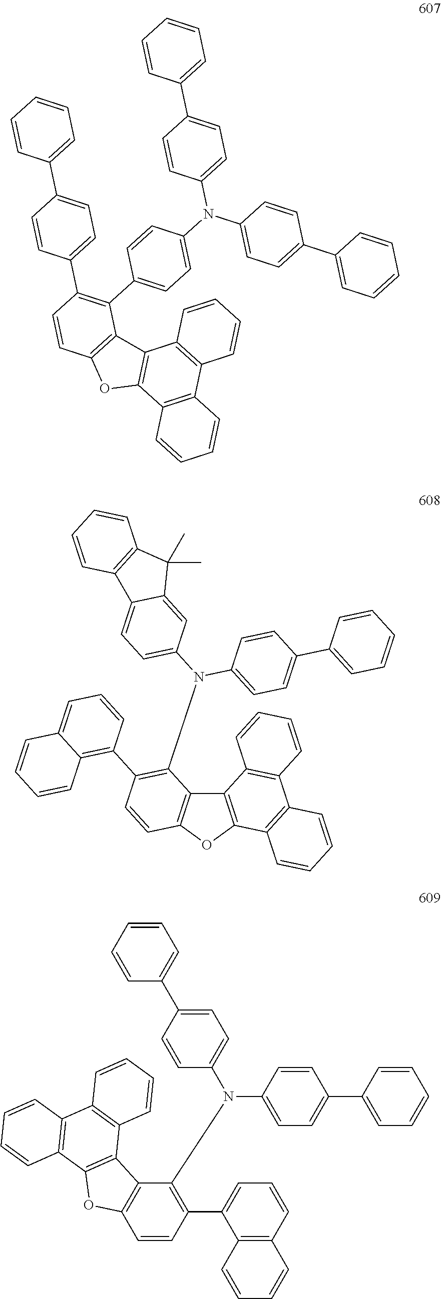 [SMILES: c1ccc(-c2ccc(-c3ccc4oc5c6ccccc6c6ccccc6c5c4c3-c3ccc(N(c4ccc(-c5ccccc5)cc4)c4ccc(-c5ccccc5)cc4)cc3)cc2)cc1 ; CC1(C)c2ccccc2-c2ccc(N(c3ccc(-c4ccccc4)cc3)c3c(-c4cccc5ccccc45)ccc4oc5c6ccccc6c6ccccc6c5c34)cc21 ; c1ccc(-c2ccc(N(c3ccc(-c4ccccc4)cc3)c3c(-c4cccc5ccccc45)ccc4oc5c6ccccc6c6ccccc6c5c34)cc2)cc1]

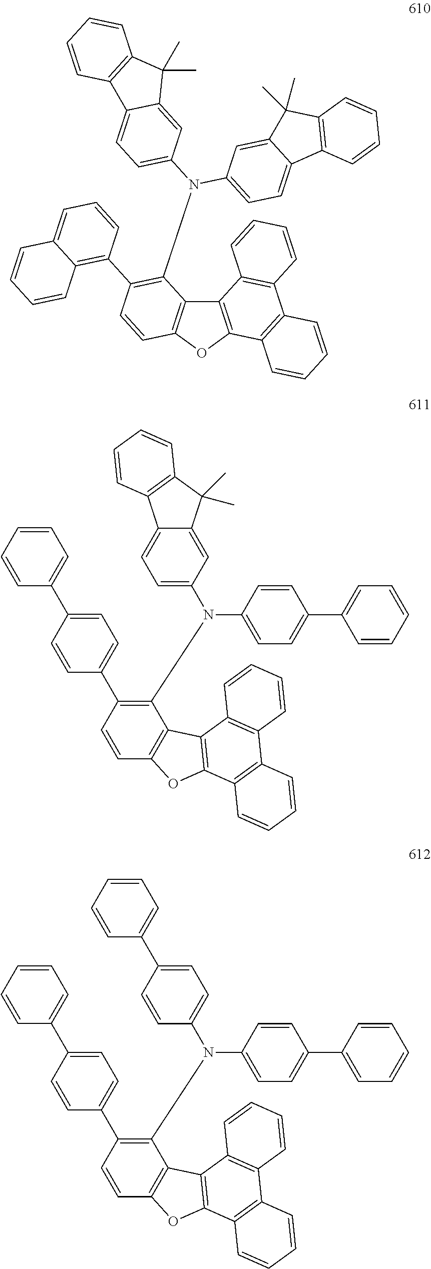 [SMILES: CC1(C)c2ccccc2-c2ccc(N(c3ccc4c(c3)C(C)(C)c3ccccc3-4)c3c(-c4cccc5ccccc45)ccc4oc5c6ccccc6c6ccccc6c5c34)cc21 ; CC1(C)c2ccccc2-c2ccc(N(c3ccc(-c4ccccc4)cc3)c3c(-c4ccc(-c5ccccc5)cc4)ccc4oc5c6ccccc6c6ccccc6c5c34)cc21 ; c1ccc(-c2ccc(-c3ccc4oc5c6ccccc6c6ccccc6c5c4c3N(c3ccc(-c4ccccc4)cc3)c3ccc(-c4ccccc4)cc3)cc2)cc1]

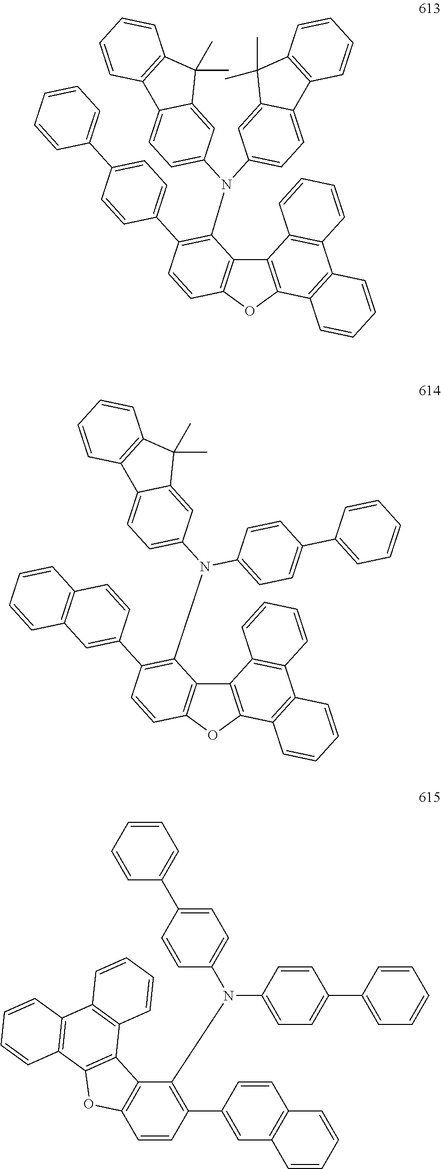 [SMILES: CC1(C)c2ccccc2-c2ccc(N(c3ccc4c(c3)C(C)(C)c3ccccc3-4)c3c(-c4ccc(-c5ccccc5)cc4)ccc4oc5c6ccccc6c6ccccc6c5c34)cc21 ; CC1(C)c2ccccc2-c2ccc(N(c3ccc(-c4ccccc4)cc3)c3c(-c4ccc5ccccc5c4)ccc4oc5c6ccccc6c6ccccc6c5c34)cc21 ; c1ccc(-c2ccc(N(c3ccc(-c4ccccc4)cc3)c3c(-c4ccc5ccccc5c4)ccc4oc5c6ccccc6c6ccccc6c5c34)cc2)cc1]

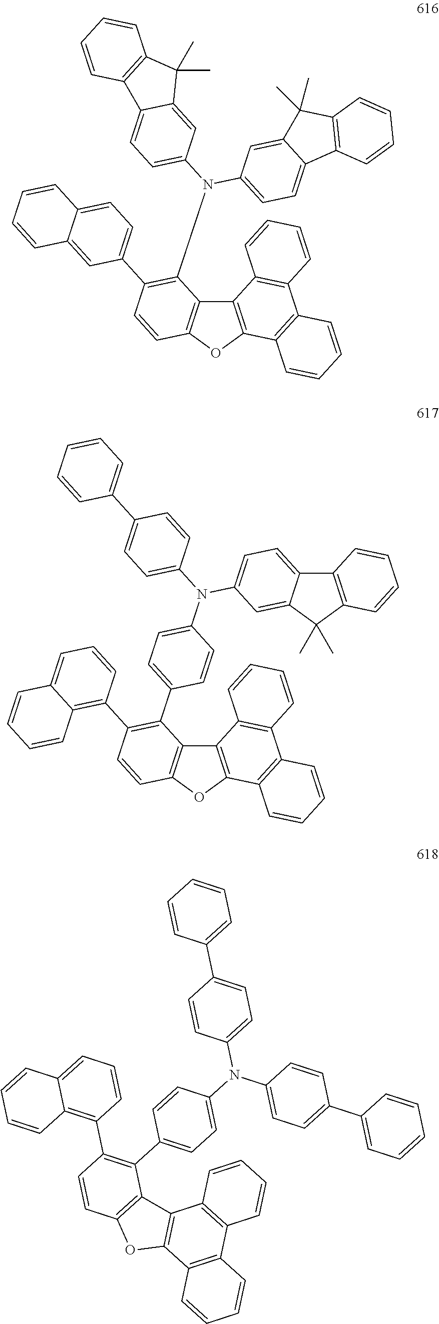 [SMILES: CC1(C)c2ccccc2-c2ccc(N(c3ccc4c(c3)C(C)(C)c3ccccc3-4)c3c(-c4ccc5ccccc5c4)ccc4oc5c6ccccc6c6ccccc6c5c34)cc21 ; CC1(C)c2ccccc2-c2ccc(N(c3ccc(-c4ccccc4)cc3)c3ccc(-c4c(-c5cccc6ccccc56)ccc5oc6c7ccccc7c7ccccc7c6c45)cc3)cc21 ; c1ccc(-c2ccc(N(c3ccc(-c4ccccc4)cc3)c3ccc(-c4c(-c5cccc6ccccc56)ccc5oc6c7ccccc7c7ccccc7c6c45)cc3)cc2)cc1]

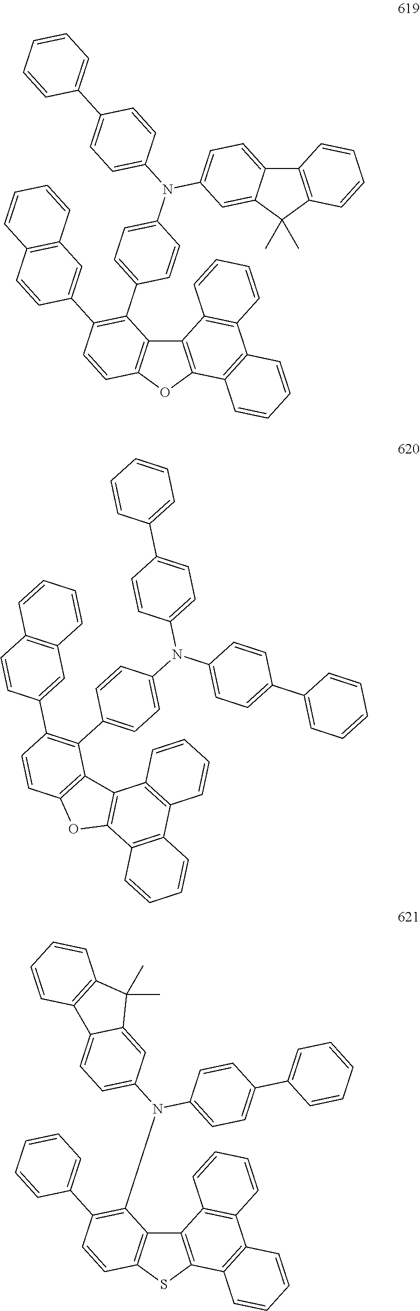 [SMILES: CC1(C)c2ccccc2-c2ccc(N(c3ccc(-c4ccccc4)cc3)c3ccc(-c4c(-c5ccc6ccccc6c5)ccc5oc6c7ccccc7c7ccccc7c6c45)cc3)cc21 ; c1ccc(-c2ccc(N(c3ccc(-c4ccccc4)cc3)c3ccc(-c4c(-c5ccc6ccccc6c5)ccc5oc6c7ccccc7c7ccccc7c6c45)cc3)cc2)cc1 ; CC1(C)c2ccccc2-c2ccc(N(c3ccc(-c4ccccc4)cc3)c3c(-c4ccccc4)ccc4sc5c6ccccc6c6ccccc6c5c34)cc21]

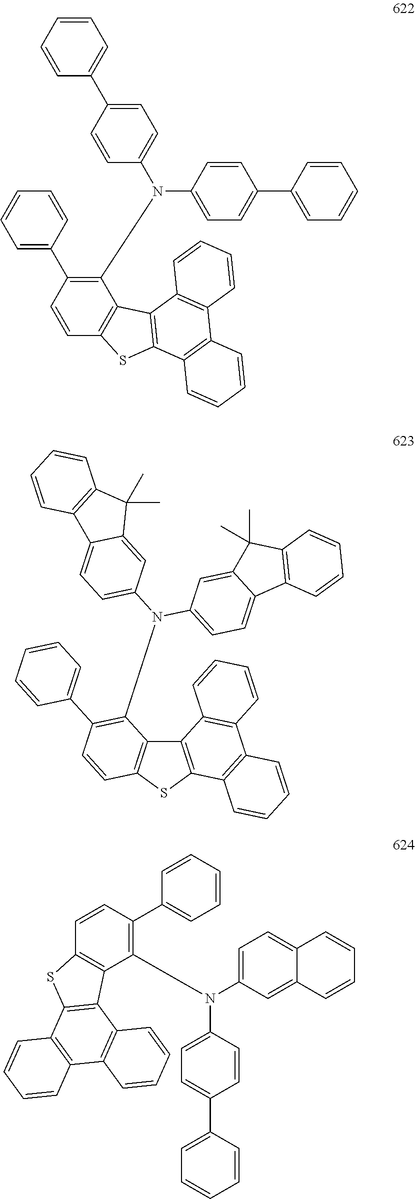 [SMILES: c1ccc(-c2ccc(N(c3ccc(-c4ccccc4)cc3)c3c(-c4ccccc4)ccc4sc5c6ccccc6c6ccccc6c5c34)cc2)cc1 ; CC1(C)c2ccccc2-c2ccc(N(c3ccc4c(c3)C(C)(C)c3ccccc3-4)c3c(-c4ccccc4)ccc4sc5c6ccccc6c6ccccc6c5c34)cc21 ; c1ccc(-c2ccc(N(c3ccc4ccccc4c3)c3c(-c4ccccc4)ccc4sc5c6ccccc6c6ccccc6c5c34)cc2)cc1]

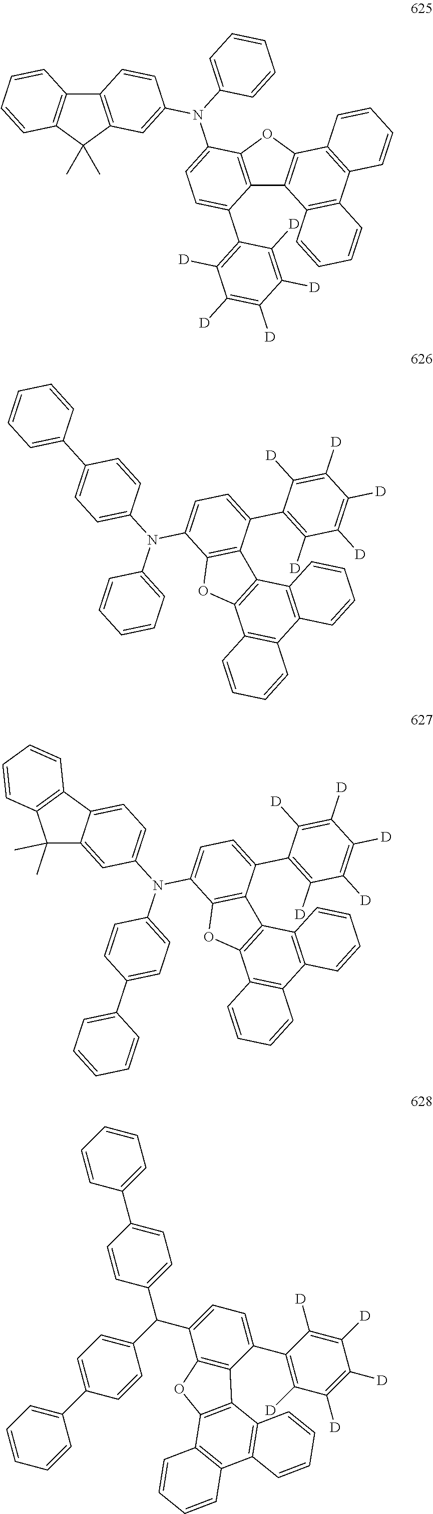 [SMILES: [2H]c1c([2H])c([2H])c(-c2ccc(N(c3ccccc3)c3ccc4c(c3)C(C)(C)c3ccccc3-4)c3oc4c5ccccc5c5ccccc5c4c23)c([2H])c1[2H] ; [2H]c1c([2H])c([2H])c(-c2ccc(N(c3ccccc3)c3ccc(-c4ccccc4)cc3)c3oc4c5ccccc5c5ccccc5c4c23)c([2H])c1[2H] ; [2H]c1c([2H])c([2H])c(-c2ccc(N(c3ccc(-c4ccccc4)cc3)c3ccc4c(c3)C(C)(C)c3ccccc3-4)c3oc4c5ccccc5c5ccccc5c4c23)c([2H])c1[2H] ; [2H]c1c([2H])c([2H])c(-c2ccc(C(c3ccc(-c4ccccc4)cc3)c3ccc(-c4ccccc4)cc3)c3oc4c5ccccc5c5ccccc5c4c23)c([2H])c1[2H]]

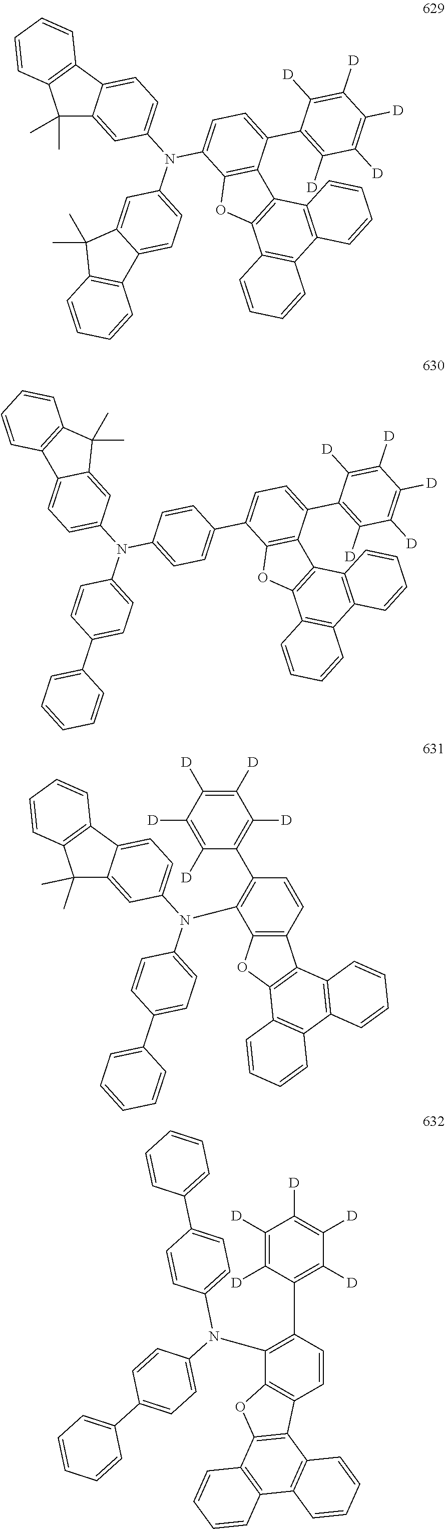

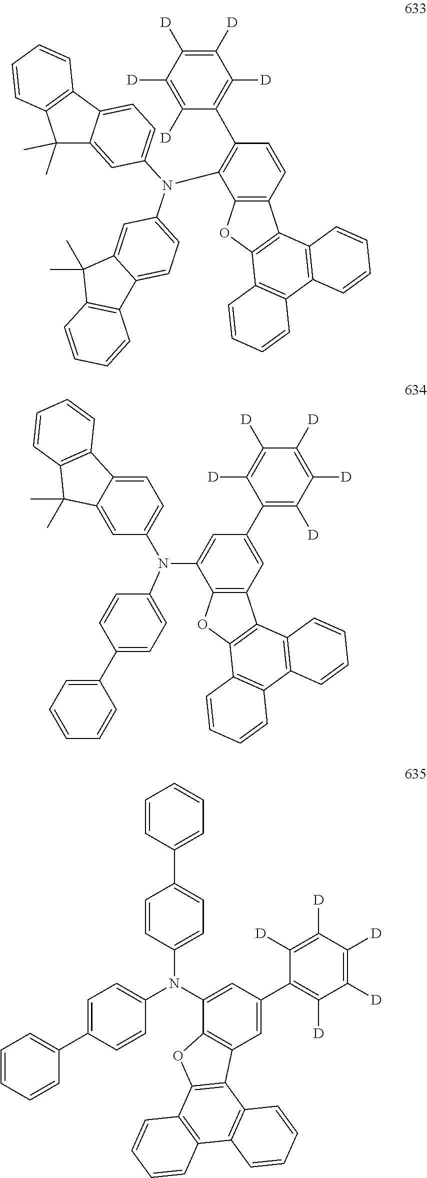

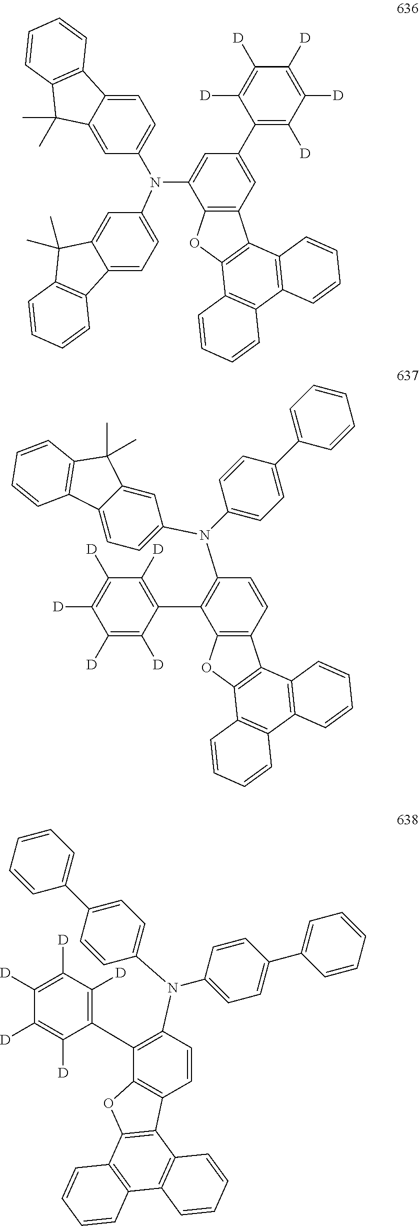 [SMILES: [2H]c1c([2H])c([2H])c(-c2cc(N(c3ccc4c(c3)C(C)(C)c3ccccc3-4)c3ccc4c(c3)C(C)(C)c3ccccc3-4)c3oc4c5ccccc5c5ccccc5c4c3c2)c([2H])c1[2H] ; [2H]c1c([2H])c([2H])c(-c2c(N(c3ccc(-c4ccccc4)cc3)c3ccc4c(c3)C(C)(C)c3ccccc3-4)ccc3c2oc2c4ccccc4c4ccccc4c32)c([2H])c1[2H] ; [2H]c1c([2H])c([2H])c(-c2c(N(c3ccc(-c4ccccc4)cc3)c3ccc(-c4ccccc4)cc3)ccc3c2oc2c4ccccc4c4ccccc4c32)c([2H])c1[2H]]

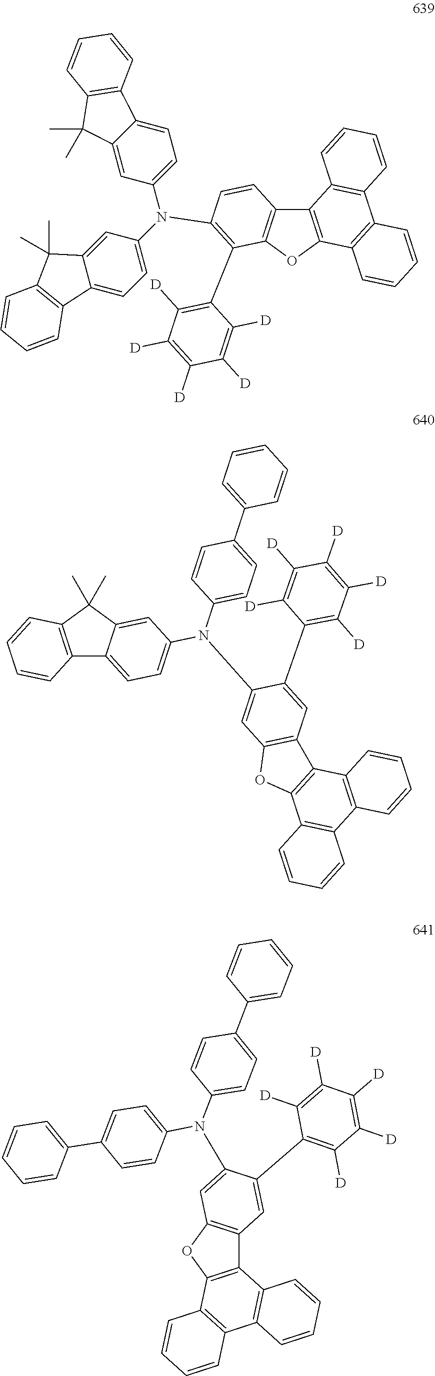 [SMILES: [2H]c1c([2H])c([2H])c(-c2c(N(c3ccc4c(c3)C(C)(C)c3ccccc3-4)c3ccc4c(c3)C(C)(C)c3ccccc3-4)ccc3c2oc2c4ccccc4c4ccccc4c32)c([2H])c1[2H] ; [2H]c1c([2H])c([2H])c(-c2cc3c(cc2N(c2ccc(-c4ccccc4)cc2)c2ccc4c(c2)C(C)(C)c2ccccc2-4)oc2c4ccccc4c4ccccc4c32)c([2H])c1[2H] ; [2H]c1c([2H])c([2H])c(-c2cc3c(cc2N(c2ccc(-c4ccccc4)cc2)c2ccc(-c4ccccc4)cc2)oc2c4ccccc4c4ccccc4c32)c([2H])c1[2H]]

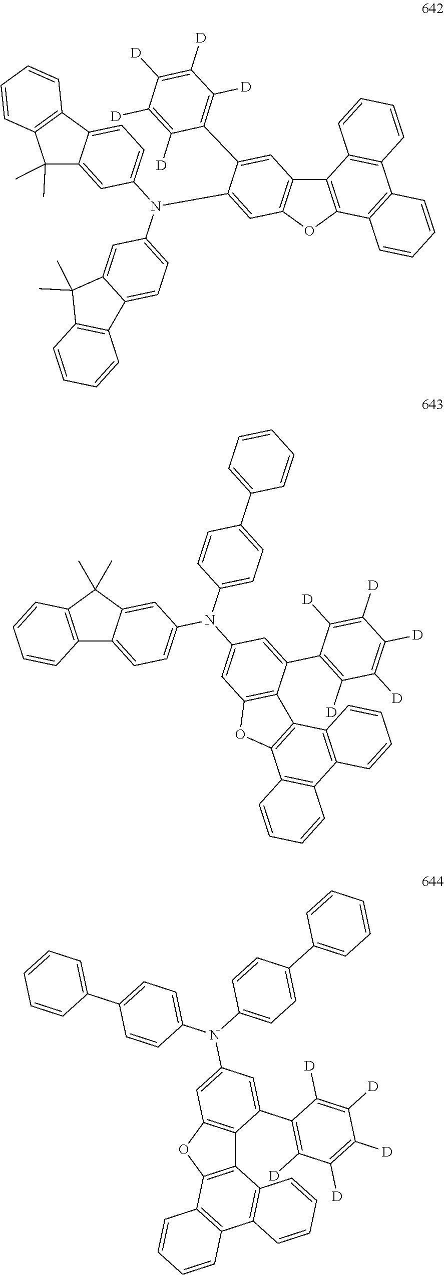 [SMILES: [2H]c1c([2H])c([2H])c(-c2cc3c(cc2N(c2ccc4c(c2)C(C)(C)c2ccccc2-4)c2ccc4c(c2)C(C)(C)c2ccccc2-4)oc2c4ccccc4c4ccccc4c32)c([2H])c1[2H] ; [2H]c1c([2H])c([2H])c(-c2cc(N(c3ccc(-c4ccccc4)cc3)c3ccc4c(c3)C(C)(C)c3ccccc3-4)cc3oc4c5ccccc5c5ccccc5c4c23)c([2H])c1[2H] ; [2H]c1c([2H])c([2H])c(-c2cc(N(c3ccc(-c4ccccc4)cc3)c3ccc(-c4ccccc4)cc3)cc3oc4c5ccccc5c5ccccc5c4c23)c([2H])c1[2H]]

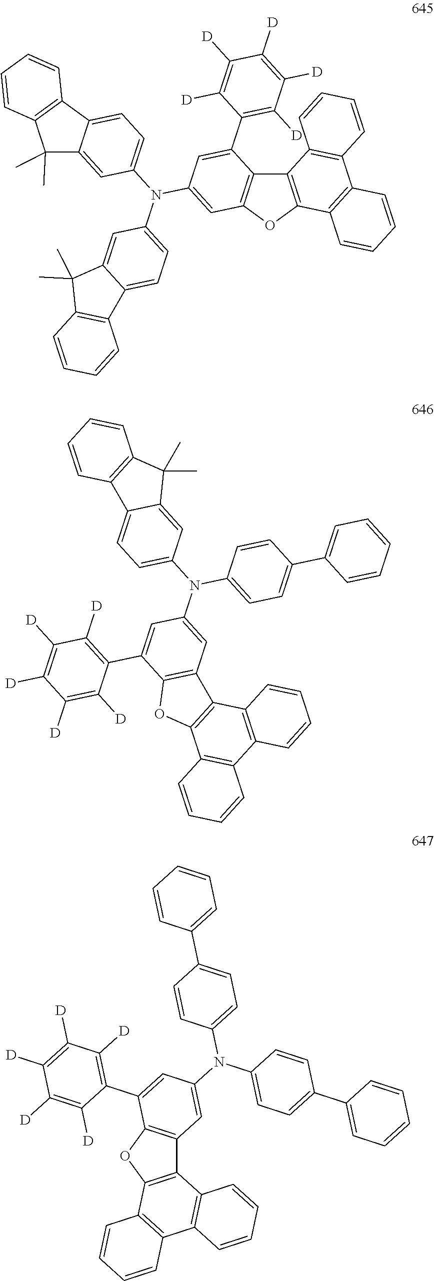

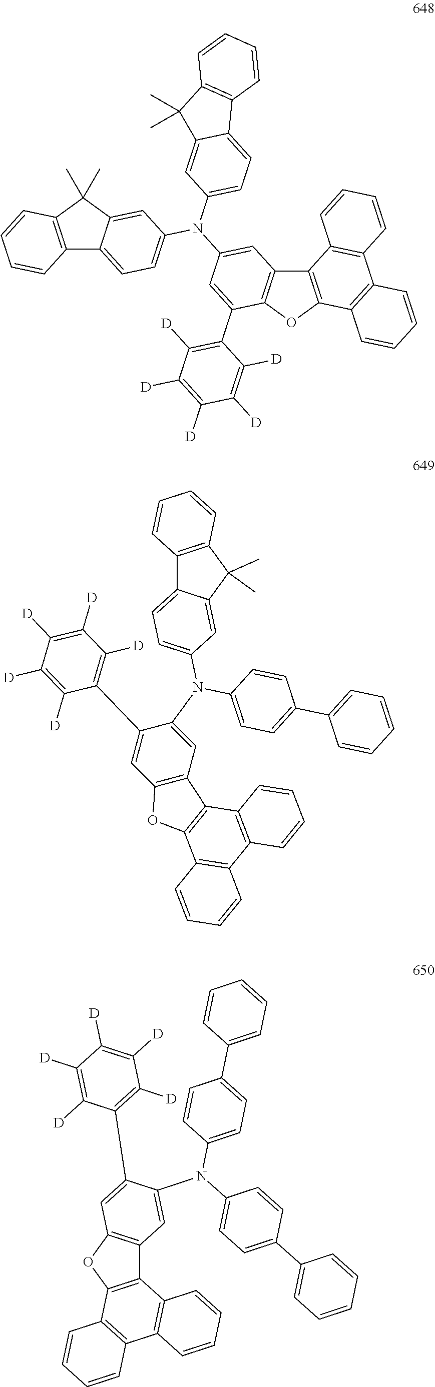 [SMILES: [2H]c1c([2H])c([2H])c(-c2cc(N(c3ccc4c(c3)C(C)(C)c3ccccc3-4)c3ccc4c(c3)C(C)(C)c3ccccc3-4)cc3c2oc2c4ccccc4c4ccccc4c32)c([2H])c1[2H] ; [2H]c1c([2H])c([2H])c(-c2cc3oc4c5ccccc5c5ccccc5c4c3cc2N(c2ccc(-c3ccccc3)cc2)c2ccc3c(c2)C(C)(C)c2ccccc2-3)c([2H])c1[2H] ; [2H]c1c([2H])c([2H])c(-c2cc3oc4c5ccccc5c5ccccc5c4c3cc2N(c2ccc(-c3ccccc3)cc2)c2ccc(-c3ccccc3)cc2)c([2H])c1[2H]]

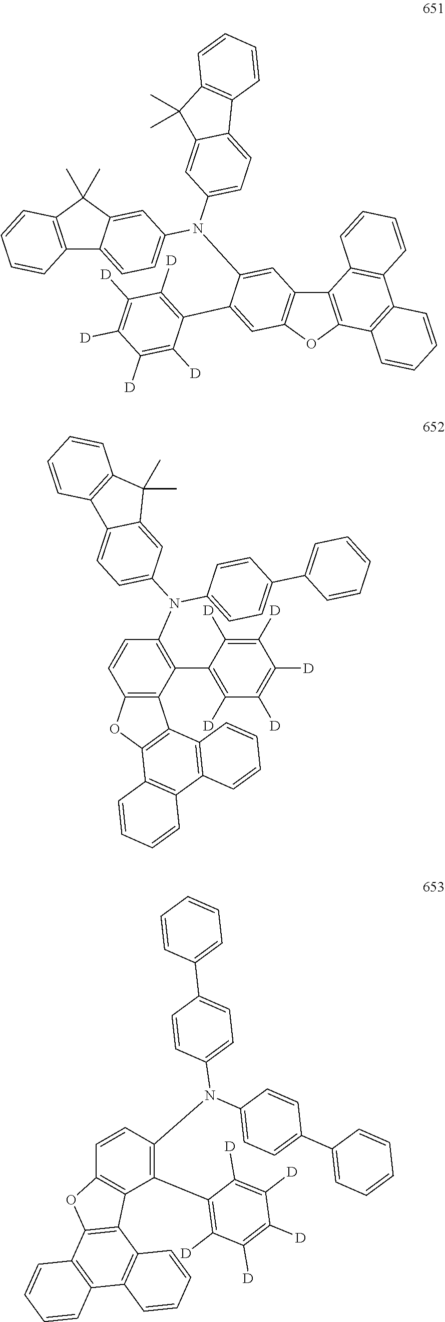 [SMILES: [2H]c1c([2H])c([2H])c(-c2cc3oc4c5ccccc5c5ccccc5c4c3cc2N(c2ccc3c(c2)C(C)(C)c2ccccc2-3)c2ccc3c(c2)C(C)(C)c2ccccc2-3)c([2H])c1[2H] ; [2H]c1c([2H])c([2H])c(-c2c(N(c3ccc(-c4ccccc4)cc3)c3ccc4c(c3)C(C)(C)c3ccccc3-4)ccc3oc4c5ccccc5c5ccccc5c4c23)c([2H])c1[2H] ; [2H]c1c([2H])c([2H])c(-c2c(N(c3ccc(-c4ccccc4)cc3)c3ccc(-c4ccccc4)cc3)ccc3oc4c5ccccc5c5ccccc5c4c23)c([2H])c1[2H]]

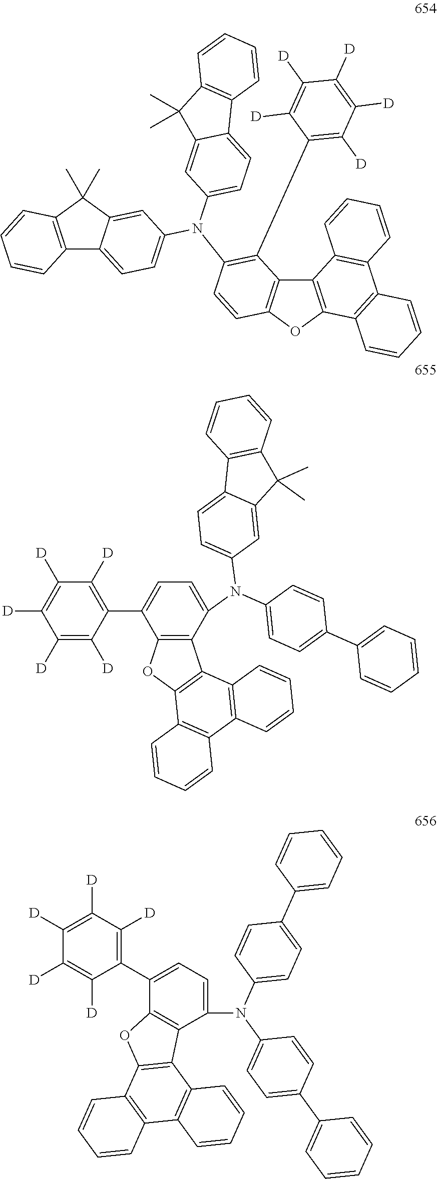 [SMILES: [2H]c1c([2H])c([2H])c(-c2c(N(c3ccc4c(c3)C(C)(C)c3ccccc3-4)c3ccc4c(c3)C(C)(C)c3ccccc3-4)ccc3oc4c5ccccc5c5ccccc5c4c23)c([2H])c1[2H] ; [2H]c1c([2H])c([2H])c(-c2ccc(N(c3ccc(-c4ccccc4)cc3)c3ccc4c(c3)C(C)(C)c3ccccc3-4)c3c2oc2c4ccccc4c4ccccc4c23)c([2H])c1[2H] ; [2H]c1c([2H])c([2H])c(-c2ccc(N(c3ccc(-c4ccccc4)cc3)c3ccc(-c4ccccc4)cc3)c3c2oc2c4ccccc4c4ccccc4c23)c([2H])c1[2H]]

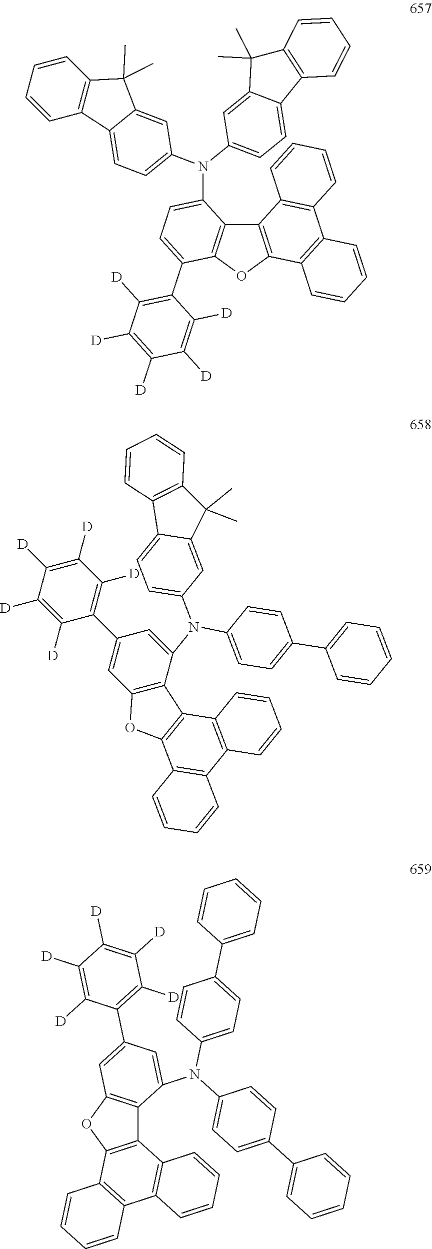 [SMILES: [2H]c1c([2H])c([2H])c(-c2ccc(N(c3ccc4c(c3)C(C)(C)c3ccccc3-4)c3ccc4c(c3)C(C)(C)c3ccccc3-4)c3c2oc2c4ccccc4c4ccccc4c23)c([2H])c1[2H] ; [2H]c1c([2H])c([2H])c(-c2cc(N(c3ccc(-c4ccccc4)cc3)c3ccc4c(c3)C(C)(C)c3ccccc3-4)c3c(c2)oc2c4ccccc4c4ccccc4c23)c([2H])c1[2H] ; [2H]c1c([2H])c([2H])c(-c2cc(N(c3ccc(-c4ccccc4)cc3)c3ccc(-c4ccccc4)cc3)c3c(c2)oc2c4ccccc4c4ccccc4c23)c([2H])c1[2H]]

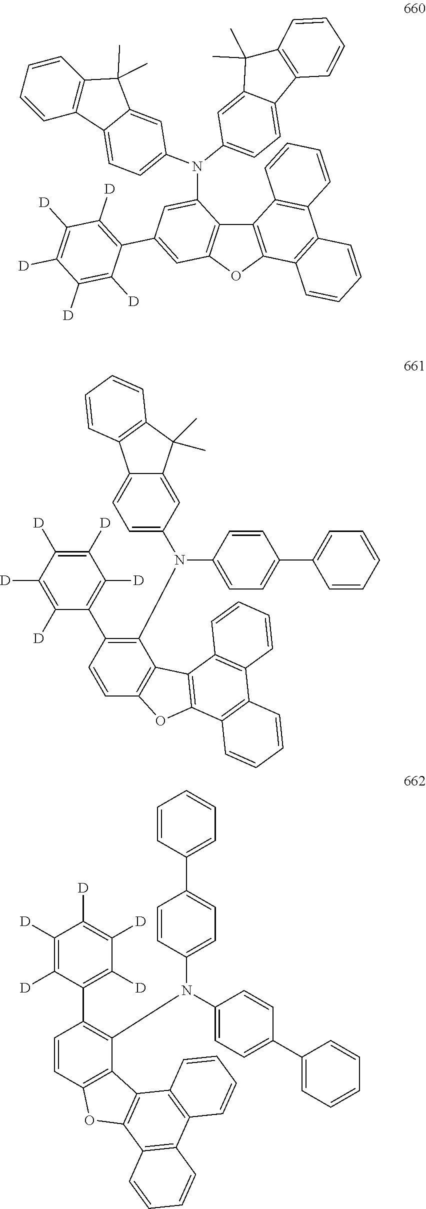 [SMILES: [2H]c1c([2H])c([2H])c(-c2cc(N(c3ccc4c(c3)C(C)(C)c3ccccc3-4)c3ccc4c(c3)C(C)(C)c3ccccc3-4)c3c(c2)oc2c4ccccc4c4ccccc4c23)c([2H])c1[2H] ; [2H]c1c([2H])c([2H])c(-c2ccc3oc4c5ccccc5c5ccccc5c4c3c2N(c2ccc(-c3ccccc3)cc2)c2ccc3c(c2)C(C)(C)c2ccccc2-3)c([2H])c1[2H] ; [2H]c1c([2H])c([2H])c(-c2ccc3oc4c5ccccc5c5ccccc5c4c3c2N(c2ccc(-c3ccccc3)cc2)c2ccc(-c3ccccc3)cc2)c([2H])c1[2H]]

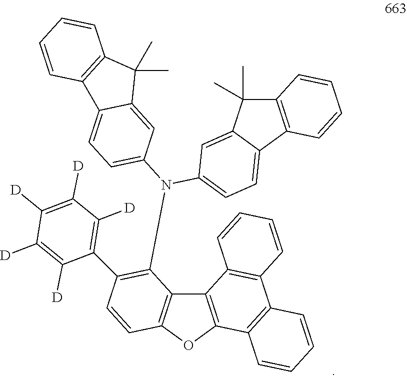 [SMILES: [2H]c1c([2H])c([2H])c(-c2ccc3oc4c5ccccc5c5ccccc5c4c3c2N(c2ccc3c(c2)C(C)(C)c2ccccc2-3)c2ccc3c(c2)C(C)(C)c2ccccc2-3)c([2H])c1[2H]]

7. An organic light-emitting device comprising:
a first electrode;
a second electrode provided to face the first electrode; and
one or more organic layers provided between the first electrode and the second electrode,
wherein the organic layers comprise the heterocyclic compound according to claim 1.

8. The organic light-emitting device according to claim 7, characterized in that the organic layer comprises a light-emitting layer and further comprises one or more layers selected from an electron injection layer, an electron transport layer, a hole-blocking layer, an electron-blocking layer, a hole transport layer, and a hole injection layer.

9. The organic light-emitting device according to claim 8, characterized in that the organic layer comprises an electron-blocking layer and a hole transport layer, and any one or more of the layers comprises the heterocyclic compound.
